(19) United States
(12) Patent Application Publication
(10) Pub. No.: US 2025/0066776 A1
(43) Pub. Date: Feb. 27, 2025

(54) ANTISENSE OLIGOMERS FOR TREATMENT OF CHRONIC KIDNEY DISEASE

(71) Applicant: Sarepta Therapeutics, Inc., Cambridge, MA (US)

(72) Inventors: Ryan OLIVER, Cambridge, MA (US); Kathy MORGAN, Cambridge, MA (US); Kevin KIM, Cambridge, MA (US); Nino JUNGELS, Cambridge, MA (US)

(73) Assignee: Sarepta Therapeutics, Inc., Cambridge, MA (US)

(21) Appl. No.: 18/647,899

(22) Filed: Apr. 26, 2024

Related U.S. Application Data
(60) Provisional application No. 63/462,389, filed on Apr. 27, 2023.

(51) Int. Cl. C12N 15/113; A61K 47/64; A61P 13/12

(52) U.S. Cl. CPC C12N 15/113; A61K 47/645; A61P 13/12; C12N 2310/11; C12N 2310/315; C12N 2310/3233

(57) ABSTRACT

Provided herein are compounds such as oligomers, peptide-oligomer-conjugates, and targeting sequences complementary to target regions within a pre-mRNA of the human uromodulin (UMOD) gene and pharmaceutical compositions thereof. Also provided herein are methods of treating a disease or disorder associated with aberrant expression of UMOD with the compounds and compositions thereof.

### CROSS REFERENCE TO RELATED APPLICATIONS

This application claims priority under 35 U.S.C. § 119 (e) to U.S. Provisional Application No. 63/462,389, filed Apr. 27, 2023, the disclosure of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 25, 2024, is named 4140_0750001_Seqlisting_ST26 and is 944,202 bytes in size.

### BACKGROUND

Antisense technology provides a means for modulating the expression of one or more specific gene products, including alternative splice products, and is uniquely useful in therapeutic, diagnostic, and research applications. The principle behind antisense technology is that an antisense compound (e.g., an oligomer) hybridizes to a target nucleic acid, and modulates gene expression activities such as transcription, splicing, or translation through one of several antisense mechanisms. The sequence specificity of antisense compounds makes them attractive as tools for target validation and gene functionalization, as well as therapeutics, to selectively modulate the expression of genes involved in disease.

Autosomal dominant tubulointerstitial kidney disease (ADTKD) is a group of rare genetic disorders characterized by tubular damage and interstitial fibrosis in the absence of glomerular lesions. Affected individuals present with progressive chronic kidney disease (CKD), normal-to-mild proteinuria, and normal-sized kidneys, often with a positive family history of autosomal dominant inheritance. ADTKD inevitably progresses to end-stage kidney disease (ESKD). The genes that cause different forms of ADTKD include UMOD, MUC1, REN, and HNF1B, and defects in these genes lead to uromodulin kidney disease (UKD), mucin-1 kidney disease, familial juvenile hyperuricemic nephropathy type 2 (FJHN2), and maturity-onset diabetes mellitus of the young type 5 (MODY5), respectively. When the cause of ADTKD is not known or a genetic test has not been performed, it is referred to as ADTKD-NOS.

Uromodulin-related autosomal-dominant tubulointerstitial kidney disease (ADTKD-UMOD) is caused by mutations in the UMOD gene, which encodes a GPI-anchored glycoprotein uromodulin (UMOD) or Tamm-Horsfall protein (THP). The clinical manifestations of ADTKD-UMOD are usually first noticed in adolescence, and progression to end stage kidney disease occurs between the third and seventh decades of life.

Treatment for ADTKD-UMOD has involved drug treatment strategies and kidney transplantation. Drug treatment strategies focus on the treatment of symptoms such as gout and hyperuricemia, and not the underlying disease. For example, many ADTKD-UMOD patients undergo lifelong treatment with allopurinol to treat and prevent gout, however, evidence that allopurinol slows the progression of the disease is minimal and insufficient. On the other hand, kidney transplantation cures ADTKD-UMOD, as the transplanted kidney does not develop the disease. However, patients are often required to wait for extended periods of time to receive a transplant, and there are inherent risks to organ transplantation. Specific therapy for inherited chronic kidney disease, including ADTKD-UMOD, by targeted delivery of a compound to diseased cells could avoid the need for invasive treatment modalities, such as kidney transplantation, and slow or halt progression of CKD while eliminating or improving symptoms of this disease.

### SUMMARY

The present disclosure relates to antisense oligomers, or pharmaceutically acceptable salts thereof, and related compositions and methods for treatment of chronic kidney disease (CKD). In certain embodiments, the chronic kidney disease is uromodulin associated autosomal dominant tubulointerstitial kidney disease (ADTKD-UMOD) (also known as uromodulin kidney disease (UKD)).

Thus, provided herein are antisense oligomers, or pharmaceutically acceptable salts thereof, comprising a non-natural chemical backbone and a targeting sequence of 13-30 bases in length that is complementary to a target region within a pre-mRNA of the human uromodulin (UMOD) gene (SEQ ID NO: 1), wherein the targeting region is an intron/exon junction or an exon internal region of the human UMOD gene pre-mRNA.

Without wishing to be bound by theory, the antisense oligomers, or pharmaceutically acceptable salts thereof, disclosed herein are useful in methods of inducing exon skipping to create premature stop codon in a human UMOD pre-mRNA, thereby triggering nonsense-mediated decay and reducing UMOD-aggregation in diseased cells. Accordingly, the antisense oligomers, or pharmaceutically acceptable salts thereof, provided herein are useful for the treatment for various conditions and symptoms in a subject in need thereof, including, but not limited to, chronic kidney disease (CKD). In certain embodiments, the chronic kidney disease is ADTKD-UMOD.

In certain embodiments, the antisense oligomer, or a pharmaceutically acceptable salt thereof, is a phosphorodiamidate morpholino oligomer (PMO). In certain embodiments, the antisense oligomer, or a pharmaceutically acceptable salt thereof, further comprises a delivery agent, including but not limited to, a cell-penetrating peptide (CPP), an antibody, an antibody fragment, an antigen binding fragment of an antibody, at least one ligand, or a combination thereof.

In an aspect provided herein, is an antisense oligomer of structural Formula (I):

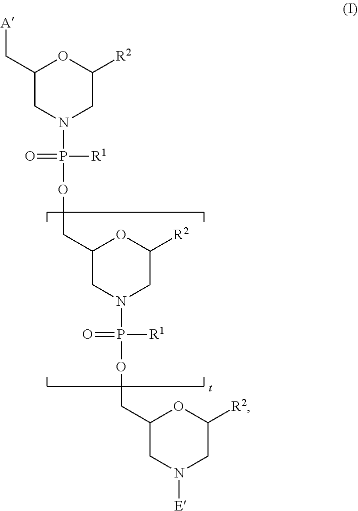

or a pharmaceutically acceptable salt thereof,
wherein:
A′ is selected from —OH,

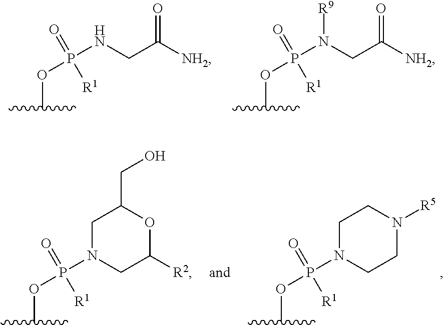

wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, —C₆₋₁₀-aryl-R⁶, 5-10 membered heteroaryl-Re, —C(O)O—(C₁₋₆-alkyl)-R⁶, —C(O)O-aryl-R⁶, —C(O)O-(5-10 membered heteroaryl)-R⁶, and

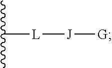

R⁶ is selected from —OH, —SH, and —NH₂, or R⁶ is O, S, or NH, each of which is covalently linked to a solid support;
R⁹ is C₁₋₆ alkyl;
each R¹ is independently selected from —OH and —N(R³)(R⁴), wherein each R³ and R⁴ is, independently at each occurrence, —H or —C₁₋₆-alkyl;
each R² is independently selected from a naturally or non-naturally occurring nucleobase, which, when taken together, forms a targeting sequence of 13 to 30 bases, that is complementary to a target region within a pre-mRNA of the human uromodulin (UMOD) gene (SEQ ID NO: 1) wherein the target region is an intron/exon junction or an exon internal region of the human UMOD gene pre-mRNA;
t is 11-28;
E′ is selected from —H, —C₁₋₆-alkyl, —C(O)C₁₋₆-alkyl, benzoyl, stearoyl, trityl, monomethoxytrityl, dimethoxytrityl, trimethoxytrityl,

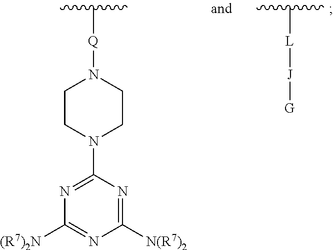

wherein:
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH) NH₂;
L is a linking amino acid, wherein L is covalently-linked by an amide bond to the C-terminus of J;
J is a cell-penetrating peptide; and
G is selected from —H, —C(O)C₁₋₆-alkyl, benzoyl, and stearoyl, wherein G is covalently-linked to J.

In another aspect provided herein, is an antisense oligomer of structural Formula (IA):

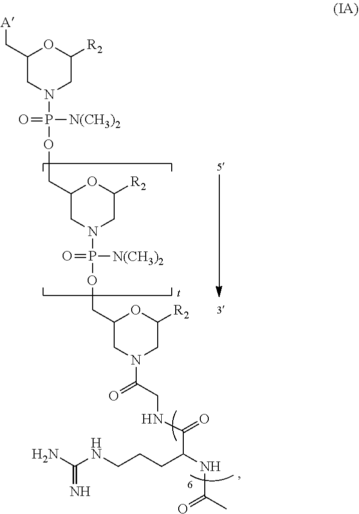

or a pharmaceutically acceptable salt thereof,
wherein:
A′ is a moiety selected from:

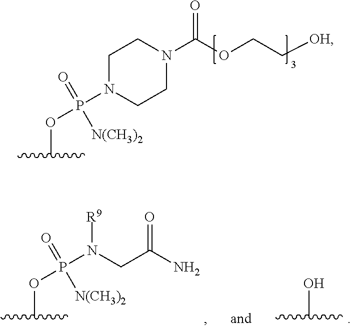

R², R⁹, and t are as described for Formula (I).

In another aspect provided herein, is an antisense oligomer of structural Formula (II):

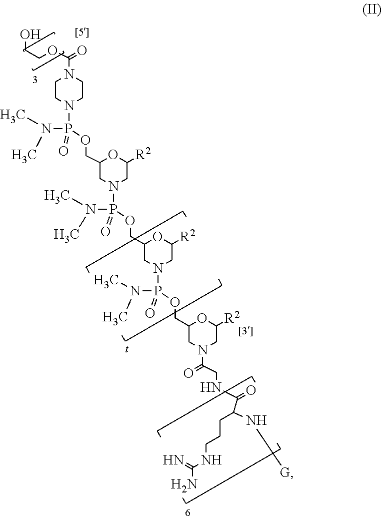

or a pharmaceutically acceptable salt thereof. t, G, and R² are as described for Formula (I).

In yet another aspect provided herein, is an antisense oligomer of structural Formula (III):

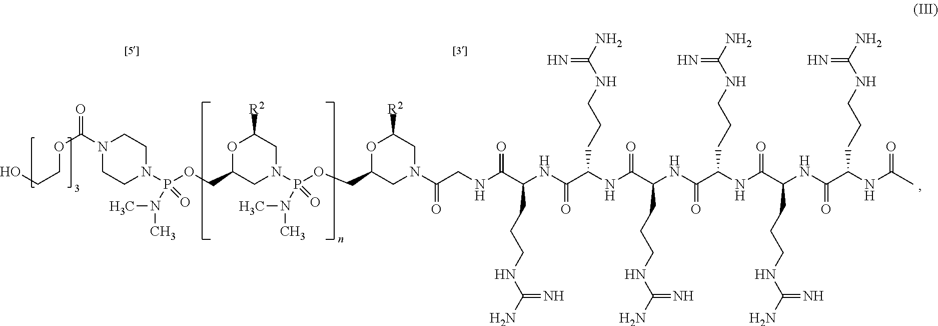

or a pharmaceutically acceptable salt thereof. n is 11-28 and R² is as described for Formula (I).

In an aspect provided herein, is an antisense oligomer of structural Formula (IV):

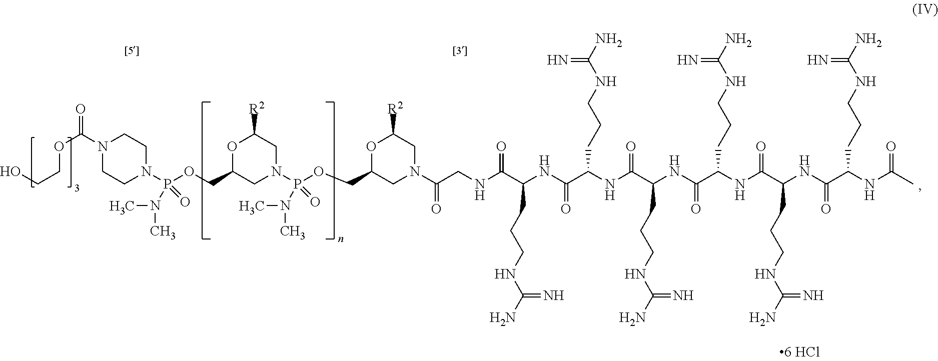

n is as described for Formula (III) and R² is as described for Formula (I).

In another aspect, provided herein is a pharmaceutical composition comprising an antisense oligomer described herein, or a pharmaceutically acceptable salt thereof, and a pharmaceutically acceptable carrier.

In an aspect provided herein, is a method of treating a disease comprising administering to a subject a therapeutically effective amount of an antisense oligomer described herein, or a pharmaceutically acceptable salt thereof. In another aspect provided herein, is a method of treating a disease comprising administering to a subject a therapeutically effective amount of a pharmaceutical composition, wherein the composition comprises an antisense oligomer described herein, or a pharmaceutically acceptable salt thereof.

In some embodiments, an antisense oligomer described herein, or a pharmaceutically acceptable salt thereof, is used for treating chronic kidney disease (CKD), including autosomal dominant tubulointerstitial kidney disease-uromodulin (ADTKD-UMOD).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A shows a bar graph depicting antisense microwalk data for various target regions within exon 2 of the human UMOD gene pre-mRNA, as described in Example 2. Individual compounds were dosed at 20 μM. FIG. 1B shows a bar graph depicting antisense microwalk data for various target regions within exon 5 of the human UMOD gene pre-mRNA. Individual compounds were dosed at 20 μM. FIG. 1C shows a bar graph depicting antisense microwalk for various target regions data within exon 6 of the human UMOD gene pre-mRNA. Individual compounds were dosed at 20 μM. FIG. 1D shows a bar graph depicting antisense microwalk for various target regions data within exon 8 of the human UM UMOD OD gene pre-mRNA. Individual compounds were dosed at 20 μM. FIG. 1E is a bar graph that shows knockdown of UMOD expression in mIMCD-3 cells treated with four different PPMOs at a single dosage. Each PPMO compound was dosed at 20 μM.

FIG. 2A shows a bar graph of knockdown of UMOD expression in mIMCD-3 cells treated with seven different PPMOs at two dosages, as described in Example 3. Each PPMO compound was dosed at 10 μM and 20 μM. FIG. 2B shows a bar graph of knockdown of UMOD expression in mIMCD-3 cells treated with three different PPMOs at varying doses. Each PPMO compound was dosed at 2.5 μM, 5 μM, 10 UM, and 20 μM.

FIG. 3A shows a photograph of a histological section of a kidney co-stained with NCC, as described in Example 4, to show distal tubular epithelial cells and EGFP to show PPMO activity. FIG. 3B shows a bar graph of PPMO activity in distal tubular epithelial cells. The compound was dosed at 10 mg/kg for low dose and at 80 mg/kg for high dose. FIG. 3C shows a photograph of a histological section of a kidney co-stained with NKCC2 to show TAL cells in Loop of Henle and EGFP to show PPMO activity. FIG. 3D shows a bar graph of PPMO activity in TAL cells. The compound was dosed at 10 mg/kg for low dose and at 80 mg/kg for high dose. FIG. 3E shows a photograph of a histological section of a kidney co-stained with UMOD and EGFP to show PPMO activity.

FIG. 4A shows a bar graph depicting UMOD mRNA expression seven days after IV injection of a UMOD-targeting PPMO. As described in Example 5, the compound was dosed at 30 mg/kg for low dose and at 100 mg/kg for high dose. FIG. 4B shows a bar graph depicting UMOD protein levels seven days after IV injection of a UMOD-targeting PPMO. the compound was dosed at 30 mg/kg for low dose and at 100 mg/kg for high dose. FIG. 4C shows a line graph depicting UMOD mRNA expression for a variable amount of time after IV injection of a UMOD-targeting PPMO. the compound was dosed at 30 mg/kg. FIGS. 4D-4E are graphic depictions of UMOD protein levels for a variable amount of time after IV injection of a UMOD-targeting PPMO. The compound was dosed at 30 mg/kg.

FIG. 5A shows a bar graph depicting UMOD protein levels in the kidneys of UMOD^{C39F} mice at 12 weeks of age, data shown were obtained as described in Example 6. FIG. 5B shows a bar graph depicting UMOD protein levels in the urine of UMOD^{C39F} mice at 12 weeks of age. FIG. 5C shows a line graph depicting urea levels in the serum of UMOD^{C39F} mice from 0 to 12 weeks of age. FIG. 5D shows a line graph depicting urea levels in the urine of UMOD^{C39F} mice from 0 to 12 weeks of age. FIG. 5E shows a bar graph depicting UMOD protein levels in the kidney of UMOD^{C39F} mice seven days after IV injection with a UMOD-targeting PPMO. The compound was dosed at 100 mg/kg.

FIG. 6 is a photograph that shows co-staining of EGFP and UMOD on a histological section of kidney to illustrate PPMO activity in UMOD-expressing cells. As described in Example 7 the compound was dosed at 80 mg/kg.

FIG. 7 is a photograph that shows co-staining of EGFP on a histological section of kidney to illustrate PPMO activity in kidney cells. As described in Example 8 the compound was dosed at 80 mg/kg.

FIG. 8A is a bar graph that shows percent change in mRNA expression over saline treatment in C57BL/6 mice. As described in Example 9 the compound was dosed at 0, 12.5, 25, 50, 100, and 150 mg/kg. The compound was dosed at 0 (saline), 12.5, 25, 50, 100, and 150 mg/kg. FIG. 8B is a bar graph that shows UMOD protein levels in C57BL/6 mice. The compound was dosed at 0 (saline), 12.5, 25, 50, 100, and 150 mg/kg. FIG. 8C shows PPMO concentration in the kidney at various PPMO doses.

FIG. 9A is a bar graph that shows UMOD protein levels in kidney. Total UMOD protein in the kidney is shown. FIG. 9B is a bar graph that shows UMOD protein levels in urine. As described in Example 10, mice were intravenously injected with either 30 mg/kg or 100 mg/kg PPMO, once or twice at one week apart, and the animals were taken down 7- or 14-days post injection. Total UMOD protein in the kidney is shown in FIGS. 9A and B.

FIG. 10A is a photograph depicting a histological section of kidney from untreated eight-week-old UMOD^{C93F} mice to demonstrate UMOD expression, as described in Example 6. FIG. 10B is a photograph that depicts a histological section of kidney from untreated one year old UMOD^{C93F} mice to demonstrate UMOD expression.

FIG. 11A is a bar graph that shows percent change in mRNA expression over saline treatment. FIG. 11B is a bar graph that shows UMOD protein levels over saline treatment in kidney.

FIG. 11C is a bar graph that shows UMOD protein levels over saline treatment in urine. As described in Example 11, mice were intravenously injected with 100 mg/kg PPMO, and the animals were taken down 7-, 14-, 21-, 28- or 42-days post injection.

FIG. 12A is a bar graph that shows the amount of PPMO in the kidney after UMOD^{C93F}/WT mice were dosed with PPMO. FIG. 12B is a bar graph that shows UMOD mRNA in the kidney after UMOD^{C93F}/WT mice were dosed with PPMO. FIGS. 12C-12F graphically depict blood chemistry panels after UMOD^{C93F}/WT mice were dosed with PPMO. FIG. 12C shows blood nitrogen urea (BUN) levels; FIG. 12D shows blood alkaline phosphatase (ALP) levels; FIG. 12E shows blood aspartate aminotransferase (AST) levels; FIG. 12F shows blood alanine aminotransferase (ALT) levels. As described in Example 12, heterozygous UMOD^{C93F} mice were intravenously injected once, twice, or four times at 30 mg/kg or 100 mg/kg PPMO and taken down after 4 weeks.

FIG. 13 illustrates the results of sequenced RNA from cells or mice treated with an exon 5 skipping PPMO to detect exon skipping. As described in Example 13, Imcd3 cells were treated with a PPMO for 3 days compared to untreated cells or mice. As further described in Example 13, WT, heterozygous, or homozygous UMOD^{C93F} mice were treated by intravenous injection with 100 mg/kg of PPMO and the kidneys were collected seven days post-injection. Total RNA extracted from cells or kidney homogenate and UMOD Ex4-6 PCR amplified. Amplicon pool subjected to amplicon sequencing. Shown junctions representing >5% reads compared to WT exon junction.

FIG. 14 is a schematic diagram that depicts PPMOs on exons 2, 5, 6, 8 and 9. The total amount of UMOD is measured with qRT-PCR with a primer set targeting exons 10 and 11. The amount of UMOD mRNA remaining after exon-skipping induced nonsense mediated decay is measured.

FIG. 15A is a bar graph that demonstrates UMOD protein in TALH cells. As described in Example 14, TALH cells were cultured on transwell plates until confluent. FIG. 15B is a bar graph that demonstrates UMOD protein in secreted media. FIG. 15C is a bar graph that demonstrates TEER (measure of electrical resistance across membrane elucidates membrane integrity). As described in Example 14 primary thick ascending limb of Henle's loop (TALH) cells were isolated and purified from human kidneys. Samples were treated with 10 μM or 30 μM of PPMO5.20 or PPMO6.6 and analyzed 48 hours later for UMOD protein levels in the cells and in the media and TEER was determined.

### DETAILED DESCRIPTION

Provided herein is an antisense oligomer, or a pharmaceutically acceptable salt thereof, comprising a non-natural chemical backbone and a targeting sequence of 13 to 30 bases in length that is complementary to a target region within a pre-mRNA of the human UMOD gene (SEQ ID NO: 1). The target region may be an intron/exon junction or an exon internal region of the human UMOD gene pre-mRNA. Such antisense oligomers, or pharmaceutically acceptable salts thereof, are useful for the treatment of various diseases, including, but not limited to, chronic kidney disease (CKD) (e.g., uromodulin associated autosomal dominant tubulointerstitial kidney disease (ADTKD-UMOD)).

Certain embodiments relate to methods of inducing exon skipping to create a premature stop codon on a human UMOD pre-mRNA in a cell, comprising contacting the cell with an antisense oligomer, or a pharmaceutically acceptable salt thereof, of sufficient length and complementarity to specifically hybridize to a region within the human UMOD gene, such that the expression of UMOD is reduced. In some embodiments, the cell is in a subject, and the method comprises administering the antisense oligomer, or a pharmaceutically acceptable salt thereof, to the subject. In certain embodiments, the method comprises contacting a cell with the antisense oligomer, or with a pharmaceutically acceptable salt thereof. In some embodiments, the cell, such as a kidney cell (e.g., kidney distal tubular cells) or muscle cell, is a diseased cell. In some embodiments, retention of UMOD protein in the endoplasmic reticulum (ER) is reduced. In some embodiments, the methods provided herein relate to reduction or abatement of: ER stress, unfolded protein response (UPR), mitochondrial dysfunction, defective protein homeostasis, autophagy in TAL cells, apoptosis of TAL epithelial cells, inflammatory lesions, fibrosis, tubular atrophy, cystic dilation, and/or progressive loss of kidney function. In some embodiments, NKCC2 activity in the TAL is restored, resulting in, for example, normal urate excretion.

In some embodiments, the antisense oligomer, or a pharmaceutically acceptable salt thereof, further comprises a delivery agent. In certain embodiments, the delivery agent facilitates delivery of a therapeutic molecular payload (e.g., an antisense oligomer) to a cell. In certain embodiments, the delivery agent increases uptake of a therapeutic molecular payload in a cell. In some embodiments, the delivery agent is a cell-penetrating peptide, an antibody, an antibody fragment, an antigen fragment of an antibody, at least one ligand, a nanocarrier, or a combination thereof. In certain embodiments, the antisense oligomer, or a pharmaceutically acceptable salt thereof, comprises a cell penetrating peptide, wherein the cell-penetrating peptide is any of the peptides provided herein or known in the art.

In some embodiments, the antisense oligomer, or a pharmaceutically acceptable salt thereof, is bonded to (e.g., covalently bonded) or is associated with (e.g., forms a complex with) a delivery agent such as a cell-penetrating peptide, an antibody, an antibody fragment, an antigen fragment of an antibody, at least one ligand, a nanocarrier, or a combination thereof.

Certain embodiments of the disclosure relate to complexes comprising a delivery agent (e.g., a cell penetrating peptide or antibody) bonded to or associated with a molecular payload (e.g., an oligomer, or pharmaceutically acceptable salt thereof). In some embodiments, the delivery agent specifically binds to an internalizing cell surface receptor on cells including but not limited to kidney cells and muscle cells. In some embodiments, the molecular payload promotes the expression or activity of a functional UMOD protein. In some embodiments, the molecular payload is an oligomer, such as an antisense oligomer, e.g., an oligomer that causes exon skipping in a mRNA expressed from a mutant UMOD-ADTKD allele. In some embodiments, the complexes provided herein are useful for delivering molecular payloads that increase or restore expression or activity of functional UMOD. Accordingly, in some embodiments, the complexes provided herein comprise delivery agents that specifically bind to receptors on the surface of cells (e.g., kidney cells) for the purpose of delivering molecular payloads to the cells. In some embodiments, the complexes are taken up into the cells via a receptor mediated internalization, following which the molecular payload is released to perform a function inside the cells. For example, complexes engineered to deliver oligomers may release the oligomers such that the oligomers can promote expression of functional UMOD (e.g., through an exon skipping mechanism) in cells (e.g., kidney cells, muscle cells, etc.). In some embodiments, the oligomers are released by cleavage (e.g., endosomal cleavage) of covalent linkers connecting oligomers and delivery agents of the complexes.

Provided herein is a pharmaceutical composition, comprising a pharmaceutically acceptable carrier; and an antisense oligomer, or a pharmaceutically acceptable salt thereof, wherein the antisense oligomer comprises a non-natural chemical backbone and a targeting sequence of 13 to 30 bases in length that is complementary to a target region within a pre-mRNA of the human UMOD gene (SEQ ID NO: 1). The target region may be an intron/exon junction or an exon internal region of the human UMOD gene pre-mRNA.

Also provided herein are methods for treating various diseases in a subject in need thereof, including, but not limited to, chronic kidney disease (CKD) (e.g., uromodulin associated autosomal dominant tubulointerstitial kidney disease (ADTKD-UMOD)).

### 1. Definitions

Unless otherwise indicated, the following terms used herein have the following meanings:

The term “about” will be understood by persons of ordinary skill in the art and will vary to some extent on the context in which it is used. As used herein when referring to a measurable value such as an amount, a temporal duration, and the like, the term “about” is meant to encompass variations of +10%.

The term “alkyl” refers to saturated, straight- or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” or “Cₓ—C_{y}” where x is the minimum and y is the maximum number of carbon atoms in the substituent. Likewise, a Cₓ chain means an alkyl chain containing x carbon atoms.

The term “heteroalkyl” by itself or in combination with another term means, unless otherwise stated, a stable straight or branched chain alkyl group consisting of the stated number of carbon atoms and up to five heteroatoms selected from O, N, S, and P, wherein the nitrogen, sulfur, and phosphorus atoms may be optionally oxidized, and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—, —O—CH₂—CH₂—CH₃, —CH₂—CH₂—CH₂—OH, —(O—CH₂—CH₂)₃—OH—, —CH₂—CH₂—NH—CH₃, —CH₂—S—CH₂—CH₃, and —CH₂—CH₂—S(═O)—CH₃. Up to three heteroatoms may be consecutive, such as, for example, —CH₂—NH—OCH₃, or —CH₂—CH₂—S—S—CH₃.

The term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. In various embodiments, examples of an aryl group may include phenyl (i.e., C₆-aryl) and biphenyl (i.e., C₁₂-aryl). In some embodiments, aryl groups have from six to sixteen carbon atoms (i.e., C₆₋₁₆-aryl). In some embodiments, aryl groups have from six to twelve carbon atoms (i.e., C₆₋₁₂-aryl). In some embodiments, aryl groups have six carbon atoms (i.e., C₆-aryl).

As used herein, the term “heteroaryl” or “heteroaromatic” refers to a heterocycle having aromatic character. Heteroaryl substituents may be defined by the number of carbon atoms, e.g., C₁₋₉-heteroaryl indicates the number of carbon atoms contained in the heteroaryl group without including the number of heteroatoms. For example, a C₁₋₉-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

The term “protecting group” or “chemical protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, monomethoxytrityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid moieties may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

Carboxylic acid and hydroxyl reactive moieties may also be blocked with hydrolytically removable protective groups such as the benzyl group, while amine groups may be blocked with base labile groups such as Fmoc. A particularly useful amine protecting group for the synthesis of compound of Formula (I) is trifluoroacetamide. Carboxylic acid reactive moieties may be blocked with oxidatively-removable protective groups such as 2,4-dimethoxybenzyl, while coexisting amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

The term “nucleobase,” “base pairing moiety,” “nucleobase-pairing moiety,” or “base” refers to the heterocyclic ring portion of a nucleoside, nucleotide, and/or morpholino subunit. Nucleobases may be naturally occurring (e.g., uracil, thymine, adenine, cytosine, and guanine), or may be modified or analogs of these naturally occurring nucleobases, e.g., one or more nitrogen atoms of the nucleobase may be independently at each occurrence replaced by carbon. Exemplary analogs include hypoxanthine (the base component of the nucleoside inosine); 2, 6-diaminopurine; 5-methyl cytosine; C₅-propynyl-modified pyrimidines; 10-(9-(aminoethoxy) phenoxazinyl) (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana (2003) RNA 9:1034-1048, Limbach et al. (1994) Nucleic Acids Res. 22:2183-2196 and Revankar and Rao, Comprehensive Natural Products Chemistry, vol. 7, 313, are also contemplated, the contents of which are incorporated herein by reference.

Further examples of base pairing moieties include, but are not limited to, expanded-size nucleobases in which one or more benzene rings have been added. Nucleic base replacements described in the Glen Research catalog (www.glenresearch.com); Krueger A T et al. (2007) Acc. Chem. Res. 40:141-150; Kool E T (2002) Acc. Chem. Res. 35:936-943; Benner S A et al. (2005) Nat. Rev. Genet. 6:553-543; Romesberg F E et al. (2003) Curr. Opin. Chem. Biol. 7:723-733; Hirao, I (2006) Curr. Opin. Chem. Biol. 10:622-627, the contents of which are incorporated herein by reference, are contemplated as useful for the synthesis of the oligomers described herein. Examples of expanded-size nucleobases are shown below:

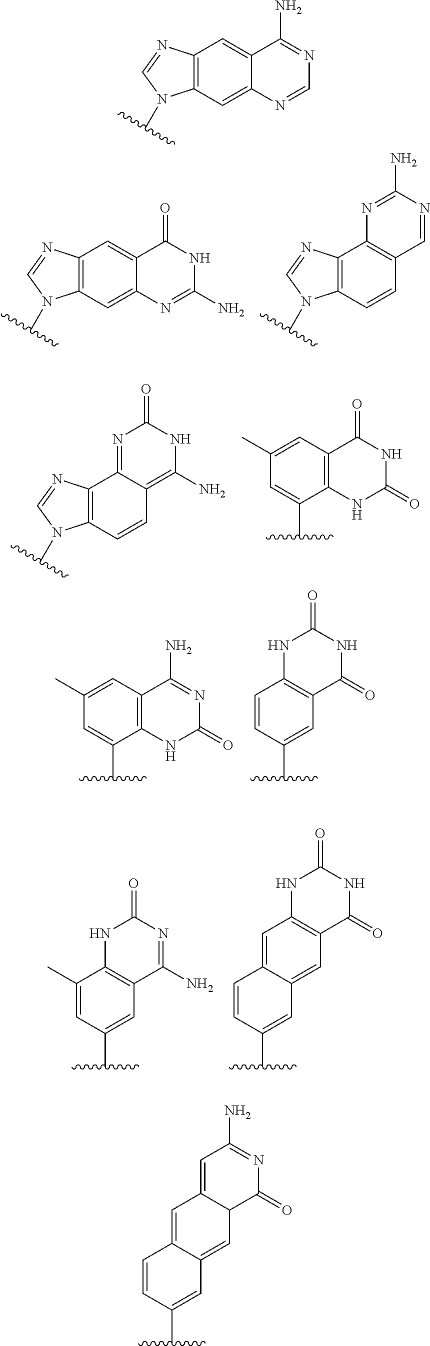

As used herein, the terms “-G-R₆” and “-G-R₆—Ac” and “R₆G” are used interchangeably and refer to a peptide moiety conjugated to an antisense oligomer, or a pharmaceutically acceptable salt thereof, of the disclosure. In various embodiments, “G” represents a glycine residue conjugated to “R₆” by an amide bond, and each “R” represents an arginine residue conjugated together by amide bonds such that “R₆” means six (6) arginine residues conjugated together by amide bonds. The arginine residues can have any stereo configuration, for example, the arginine residues can be L-arginine residues, D-arginine residues, or a mixture of D- and L-arginine residues. “Ac” represents an acetyl group conjugated to the C-terminal R residue. In certain embodiments, “-G-R₆” or “-G-R₆—Ac” is conjugated to the morpholine ring nitrogen of 3′ most morpholino subunit of a PMO antisense oligomer, or a pharmaceutically acceptable salt thereof, of the disclosure. In some embodiments, “-G-R₆” or “-G-R₆—Ac” is conjugated to the 3′ end of an antisense oligomer, or a pharmaceutically acceptable salt thereof, of the disclosure and is of the following formula:

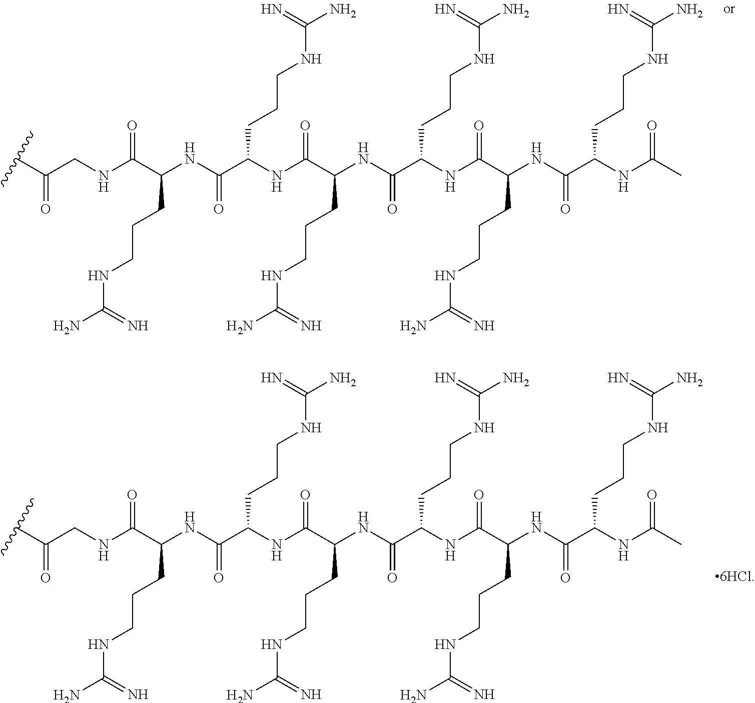 [SMILES: *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O ; *C(=O)CNC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(C)=O]

The terms “oligonucleotide” or “oligomer” refer to a compound comprising a plurality of linked nucleosides, nucleotides, or a combination of both nucleosides and nucleotides. In certain embodiments provided herein, an oligomer is a morpholino oligomer. It is to be understood that the terms “oligonucleotide” or “oligomer” include pharmaceutically acceptable salts thereof (e.g., acid addition salts such as HCl salts).

As used herein, the terms “antisense oligomer” or “antisense compound” are used interchangeably and refer to a sequence of subunits, each having a base carried on a backbone subunit composed of ribose or other pentose sugar or morpholino group, and where the backbone groups are linked by intersubunit linkages that allow the bases in the compound to hybridize to a target sequence in a nucleic acid (typically RNA) by Watson-Crick base pairing, to form a nucleic acid:oligomer heteroduplex within the target sequence. The oligomer may have exact sequence complementarity to the target sequence or nearly exact complementarity. Such antisense oligomers, or pharmaceutically acceptable salts thereof, are designed to block or inhibit translation of the mRNA containing the target sequence and may be said to be “directed to” a sequence with which it hybridizes.

Also contemplated herein as types of “antisense oligomers” or “antisense compounds” including pharmaceutically acceptable salts thereof, are phosphorothioate-modified oligomers, peptide nucleic acids (PNAs), locked nucleic acids (LNAs), 2′-fluoro-modified oligomers, 2′-0,4′-C-ethylene-bridged nucleic acids (ENAs), tricyclo-DNAs, tricylo-DNA phosphorothioate-modified oligomers, 2′-O-[2-(N-methylcarbamoyl) ethyl] modified oligomers, 2′-O-methyl phosphorothioate modified oligomers, 2′-O-methoxyethyl (2′-O-MOE) modified oligomers, and 2′-O-methyl oligomers, or combinations thereof, as well as other antisense agents known in the art.

An antisense oligomer, or a pharmaceutically acceptable salt thereof, “specifically hybridizes” to a target polynucleotide if the oligomer hybridizes to the target under physiological conditions, with a Tm greater than 37° C., greater than 45° C., at least 50° C., and typically 60° C.-80° C. or higher. The “Tm” of an oligomer is the temperature at which 50% hybridizes to a complementary polynucleotide. Tm is determined under standard conditions in physiological saline, as described, for example, in Miyada et al. (1987) Methods Enzymol. 154:94-107. Such hybridization may occur with “near” or “substantial” complementarity of the antisense oligomer, or a pharmaceutically acceptable salt thereof, to the target sequence, as well as with exact complementarity.

The terms “complementary” and “complementarity” refer to oligomers (i.e., a sequence of nucleotides) related by base-pairing rules. For example, the sequence “T-G-A (5′-3′)” is complementary to the sequence “T-C-A (5′-3′).” Complementarity may be “partial,” in which only some of the nucleic acids' bases are matched according to base pairing rules, or there may be “complete,” “total,” or “perfect” (100%) complementarity between the nucleic acids. The degree of complementarity between nucleic acid strands has significant effects on the efficiency and strength of hybridization between nucleic acid strands. In some embodiments, oligomers can include one or more mismatches (e.g., 6, 5, 4, 3, 2, or 1 mismatches) with respect to the target RNA. Such hybridization may occur with “near” or “substantial” complementarity of the antisense oligomer to the target sequence, as well as with exact complementarity. In some embodiments, an oligomer may hybridize to a target sequence with about 50%, 55%, 60%, 65%, 70%, 75%, 80%, 85%, 90%, 95%, 99% or 100% complementarity. Variations at any location within the oligomer are included. In certain embodiments, variations in sequence near the termini of an oligomer, if present, are typically within about 6, 5, 4, 3, 2, or 1 nucleotides of 5′-terminus, 3′-terminus, or both termini.

The terms “TEG,” “EG3,” or “triethylene glycol tail” refer to triethylene glycol moieties conjugated to the oligomer, e.g., at its 3′- or 5′-end. For example, in some embodiments, “TEG” includes, for example, wherein A′ of the conjugate of Formula (I) is of the formula:

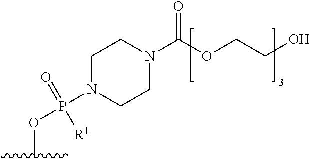

Naturally occurring nucleotide bases include adenine, guanine, cytosine, thymine, and uracil, which have the symbols A, G, C, T, and U, respectively. Nucleotide bases can also encompass analogs of naturally occurring nucleotide bases. Base pairing typically occurs between purine A and pyrimidine T or U, and between purine G and pyrimidine C.

Oligomers may also include nucleobase modifications or substitutions. Oligomers containing a non-natural or substituted base include oligomers in which one or more purine or pyrimidine bases most commonly found in nucleic acids are replaced with less common or non-natural bases. In some embodiments, the nucleobase is covalently linked at the N9 atom of the purine base, or at the N1 atom of the pyrimidine base, to the morpholine ring of a nucleotide or nucleoside.

Purine bases comprise a pyrimidine ring fused to an imidazole ring, as described by the general formula:

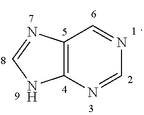

Adenine and guanine are the two purine nucleobases most commonly found in nucleic acids. These may be substituted with other naturally occurring purines, including, but not limited to, N6-methyladenine, N2-methylguanine, hypoxanthine, and 7-methylguanine.

Pyrimidine bases comprise a six-membered pyrimidine ring as described by the general formula:

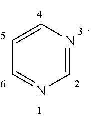

Cytosine, uracil, and thymine are the pyrimidine bases most commonly found in nucleic acids. These may be substituted with other naturally occurring pyrimidines, including, but not limited to, 5-methylcytosine, 5-hydroxymethylcytosine, pseudouracil, and 4-thiouracil. In one embodiment, the oligomers described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Pseudouracil is a naturally occurring isomerized version of uracil, with a C-glycoside rather than the regular N-glycoside as in uridine.

In some embodiments, modified or substituted nucleobases are useful for facilitating the purification of antisense oligomers. For example, in certain embodiments, antisense oligomers may contain three or more (e.g., 3, 4, 5, 6, or more) consecutive guanine bases. In certain antisense oligomers, a string of three or more consecutive guanine bases can result in aggregation of the oligomers, complicating purification. In such antisense oligomers, one or more of the consecutive guanines can be substituted with hypoxanthine. The substitution of hypoxanthine for one or more guanines in a string of three or more consecutive guanine bases can reduce aggregation of the antisense oligomers, thereby facilitating purification.

The oligomers provided herein are synthesized and do not include antisense compositions of biological origin. The molecules of the disclosure may also be mixed, encapsulated, conjugated, or otherwise associated with other molecules, molecule structures, or mixtures of compounds, as for example, liposomes, receptor-targeted molecules, oral, rectal, topical, or other formulations, for assisting in uptake, distribution, or absorption, or a combination thereof.

As used herein, a “nucleic acid analog” refers to a non-naturally occurring nucleic acid molecule. A nucleic acid is a polymer of nucleotide subunits linked together into a linear structure. Each nucleotide consists of a nitrogen-containing aromatic base attached to a pentose (five-carbon) sugar, which is in turn attached to a phosphate group. Successive phosphate groups are linked together through phosphodiester bonds to form the polymer. The two common forms of naturally occurring nucleic acids are deoxyribonucleic acid (DNA) and ribonucleic acid (RNA). One end of the chain carries a free phosphate group attached to 5′-carbon atom of a sugar moiety; this is called the 5′ end of the molecule. The other end has a free hydroxyl (—OH) group at 3′-carbon of a sugar moiety and is called 3′ end of the molecule. A nucleic acid analog can include one or more non-naturally occurring nucleobases, sugars, and/or internucleotide linkages, for example, a phosphorodiamidate morpholino oligomer (PMO). As disclosed herein, in certain embodiments, a “nucleic acid analog” is a PMO, and in certain embodiments, a “nucleic acid analog” is a positively charged cationic PMO.

A “morpholino oligomer” refers to a polymeric molecule having a backbone that supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. An exemplary “morpholino” oligomer comprises morpholino subunit structures linked together by phosphoramidate or phosphorodiamidate linkages, joining the morpholino nitrogen of one subunit to 5′ exocyclic carbon of an adjacent subunit, each subunit comprising a purine or pyrimidine base-pairing moiety effective to bind, by base-specific hydrogen bonding, to a base in a polynucleotide. Morpholino oligomers (including antisense oligomers) are detailed, for example, in U.S. Pat. Nos. 5,034,506; 5,142,047; 5,166,315; 5,185,444; 5,217,866; 5,506,337; 5,521,063; 5,698,685; 8,076,476; and 8,299,206; and PCT publication number WO 2009/064471, each of which is incorporated herein by reference in its entirety.

A preferred morpholino oligomer is a phosphorodiamidate-linked morpholino oligomer, referred to herein as a PMO. Such oligomers are composed of morpholino subunit structures such as those shown below:

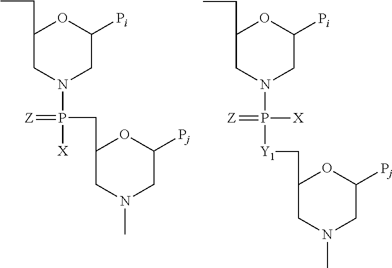

where X is NH₂, NHR, or NR₂ (where R is lower alkyl, (e.g., methyl, ethyl, propyl, etc.)), Y1 is O, and Z is O, and Pi and Pj are purine or pyrimidine base-pairing moieties effective to bind, by base-specific hydrogen bonding, to a base in a polynucleotide. Also contemplated herein are structures having an alternate phosphorodiamidate linkage, where X is lower alkoxy, such as methoxy or ethoxy, Y1 is NH or NR, where R is lower alkyl, and Z is O.

Representative PMOs include PMOs wherein the intersubunit linkages are linkage (A1) as shown in Table 1.

TABLE 1
Representative Intersubunit Linkages

| No. | Name | Structure |
|---|---|---|
| A1 | PMO | 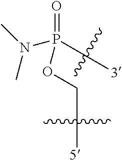 |
| A2 | PMO⁺ (unprotonated form depicted) | 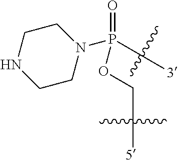 |
| A3 | PMO⁺ (+) | 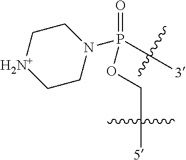 |

A “phosphoramidate” group comprises phosphorus having three attached oxygen atoms and one attached nitrogen atom, while a “phosphorodiamidate” group comprises phosphorus having two attached oxygen atoms and two attached nitrogen atoms. A representative phosphorodiamidate example is below:

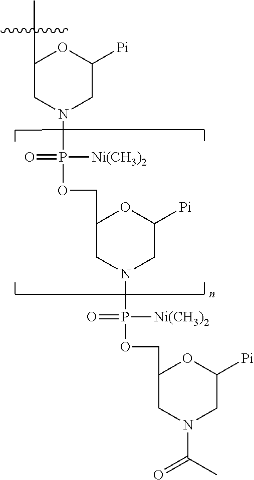

where each Pᵢ is independently selected from H, a nucleobase, and a nucleobase functionalized with a chemical protecting-group, wherein the nucleobase independently at each occurrence comprises a pyridine, a pyrimidine, a triazinane, purine, or a deaza-purine; and n is an integer from 6 to 38 (i.e., n is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38). In certain embodiments, n is an integer from 11 to 28. The ring nitrogen of the subunit at the 3′ terminus of a PMO may be capped with a capping group such as acetyl or may be uncapped with a free hydrogen.

In the uncharged or the modified intersubunit linkages of the antisense oligomers described herein, one nitrogen is always pendant to the backbone chain. The second nitrogen, in a phosphorodiamidate linkage, is typically the ring nitrogen in a morpholino ring structure.

PMOs are water-soluble, uncharged, or substantially uncharged antisense molecules that inhibit gene expression by preventing binding or progression of splicing or translational machinery components. PMOs have also been shown to inhibit or block viral replication (Stein, Skilling et al. 2001; McCaffrey, Meuse et al. 2003), and are highly resistant to enzymatic digestion (Hudziak, Barofsky et al. 1996). PMOs have demonstrated high antisense specificity and efficacy in vitro in cell-free and cell culture models (Stein, Foster et al. 1997; Summerton and Weller 1997), and in vivo in zebrafish, frog, and sea urchin embryos (Heasman, Kofron et al. 2000; Nasevicius and Ekker 2000), as well as in adult animal models, such as rats, mice, rabbits, dogs, and pigs (see e.g. Arora and Iversen 2000; Qin, Taylor et al. 2000; Iversen 2001; Kipshidze, Keane et al. 2001; Devi 2002; Devi, Oldenkamp et al. 2002; Kipshidze, Kim et al. 2002; Ricker, Mata et al. 2002).

Antisense PMO oligomers have been shown to be taken up into cells and to be more consistently effective in vivo, with fewer nonspecific effects, than other widely used antisense oligomers (see e.g., P. Iversen, “Phosphoramidite Morpholino Oligomers,” in Antisense Drug Technology, S. T. Crooke, ed., Marcel Dekker, Inc., New York, 2001). Conjugation of PMOs to arginine-rich peptides has been shown to increase their cellular uptake (see, e.g., U.S. Pat. No. 7,468,418, incorporated herein by reference in its entirety).

“Charged,” “uncharged,” “cationic,” and “anionic” as used herein refer to the predominant state of a chemical moiety at near-neutral pH, e.g., about 6 to 8. For example, the term may refer to the predominant state of the chemical moiety at physiological pH, that is, at about 7.4.

A “cationic PMO” or “PMO+” refers to a phosphorodiamidate morpholino oligomer comprising any number of (1-piperazino)phosphinylideneoxy, (1-(4-(ω-guanidino-alkanoyl))-piperazino) phosphinylideneoxy linkages (A2 and A3; see Table 1) that have been described previously (see e.g., PCT publication WO 2008/036127, which is incorporated herein by reference in its entirety).

The “backbone” of an antisense oligomer (e.g., an uncharged oligomer analog) refers to the structure supporting the base-pairing moieties; e.g., for a morpholino oligomer, as described herein, the “backbone” includes morpholino ring structures connected by intersubunit linkages (e.g., phosphorus-containing linkages). A “substantially uncharged backbone” refers to the backbone of an oligomer analog wherein less than 50% of the intersubunit linkages are charged at near-neutral pH. For example, a substantially uncharged backbone may comprise less than 50%, less than 40%, less than 30%, less than 20%, less than 10%, less than 5% or even 0% intersubunit linkages which are charged at near neutral pH. In some embodiments, the substantially uncharged backbone comprises at most one charged (at physiological pH) intersubunit linkage for every four uncharged (at physiological pH) linkages, at most one for every eight or at most one for every sixteen uncharged linkages. In some embodiments, the nucleic acid analogs described herein are fully uncharged. In certain embodiments, the antisense oligomers, or a pharmaceutically acceptable salt thereof, disclosed herein comprise internucleotidic linkages (i.e., backbones) selected from phosphorothioate linkages, phosphodiester linkages, N-(1,3-dimethylimidazolidin-2-ylidenyl)phosphoramidate linkages (n001), and combinations thereof. In certain embodiments of the oligomers disclosed herein, the linkage phosphorus of each phosphorothioate linkage is Sp, and the linkage phosphorus of each n001 linkage is Rp. In some embodiments, the oligomers disclosed herein further comprise certain chemistry moieties such as 2′-F, 2′-OMe, etc.

In certain embodiments, the compounds described herein comprise or consist of an oligomer, or a pharmaceutically acceptable salt thereof, comprising a targeting (base) sequence that is complementary to a target nucleic acid. In certain embodiments, the target nucleic acid is an endogenous RNA molecule. In certain embodiments, the target nucleic acid encodes a protein. In certain embodiments, the target nucleic acid is non-coding. In certain such embodiments, the target nucleic acid is selected from an mRNA and a pre-mRNA, including intronic, exonic, intronic/exonic junctions, and untranslated regions. In certain embodiments, the target RNA is an mRNA. In certain embodiments, the target nucleic acid is a pre-mRNA. In certain embodiments, the target region is entirely within an exon. In certain embodiments, the target region is entirely within an intron. In certain embodiments, the target region spans an intron/exon junction. In certain embodiments, the target region is at least 50% within an intron.

The term “targeting base sequence” or “targeting sequence” is the (nucleobase) sequence in the antisense oligomer that is complementary or substantially complementary to a target nucleic acid. The entire sequence, or only a portion, of the oligomer compound may be complementary to the target sequence.

The “target region” refers to a sequence in a nucleic acid that is targeted by, and is therefore complementary, substantially complementary, or partially complementary, to the targeting sequence of the antisense oligomer. The entire sequence, or only a portion, of the target region may be complementary to the targeting sequence of the oligomer compound described herein. In certain embodiments, a plurality of antisense compounds is directed to a single target region. In certain embodiments, a nomenclature system according to gene, species, exon number, and annealing coordinates is used herein to identify or indicate a target region or annealing coordinates of a targeted nucleic acid (e.g., a nucleic acid transcript) as follows:

G SpZA/D(±X±Y),
where G is gene name;
Sp is species (e.g., “H” for human, “M” for mouse);
Z is exon number;
A/D is selected from acceptor (A) and donor (D);
The symbol “−” denotes an intronic position;
The symbol “+” denotes an exonic position;
X is the position of the first 5′ base relative to A or D site; and
Y is the position of the first 3′ base relative to A or D site.

The nomenclature begins with the name of the transcript (e.g., uromodulin; UMOD), then the species of the target mRNA (e.g., H: human or M: mouse), followed by the target exon number of the specified transcript, and specification of an acceptor (A) or donor (D) site. The annealing coordinates are shown in brackets within the pre-mRNA transcript. The intronic bases are designated with a negative prefix (−) and the exonic position with a positive (+) symbol. The annealing coordinates are the positions of bases relative to the bases relative to the acceptor or donor sites of the reference transcript as denoted by National Center for Biotechnology Information and Ensembl genome browser 96. See, for example, international application WO2006/000057; Aung-Htut, et al. (2019) Int. J. Mol. Sci. 20:5030; and Mann, et al. (2002) J Gene Med. 4:644.

As an example, entirely exonic annealing coordinates represented by UMOD H2A(+108+127) indicate the site within the 108^{th} and 127^{th} nucleotides as measured from the 5′ end of an exon 2 (e.g., exon 2 of human UMOD gene pre-mRNA) (i.e., the region within the 108^{th} and 127^{th} nucleotides as measured from the start of exon 2 at the 5′ end). The closest splice site is the acceptor; therefore, these coordinates are preceded with “A.”

As another example, UMOD H5D (+18-2) indicates the last 18 exonic bases and the first 2 intronic bases of the target exon donor splice site (i.e., the target region within the 18^{th} nucleotide of exon 5 measured from 3′ end of exon 5 to the 2nd nucleotide of intron 5 measured from 3′ end of exon 5 (e.g., human exon 5 of the human UMOD gene pre-mRNA)). The closest splice site is the donor; therefore, these coordinates are preceded with “D.”

As another example, UMOD H5A (−11+9) indicates the last 11 bases of the intron preceding the target exon 5 and the first 9 bases of the target exon 5 (i.e., the target region within the 11^{th} nucleotide of intron 4 measured from the 5′ end of exon 5 to the 9^{th} nucleotide of exon 5 measured from 5′ end of exon 5 (e.g., human exon 5 of the human UMOD gene pre-mRNA)). The closest splice site is the acceptor; therefore, these coordinates are preceded with “A.”

The terms “peptide” and “polypeptide” refer to a compound comprising a plurality of linked amino acids. In certain embodiments, the peptides provided herein are cell-penetrating peptides (CPP). In certain embodiments, the polypeptides provided herein are antibodies or fragments thereof.

As used herein, a “cell-penetrating peptide” (CPP) or “carrier peptide” is a relatively short peptide capable of promoting the uptake of PMOs by cells, thereby delivering the PMOs to the interior (cytoplasm) of the cells. In some embodiments, the CPP or carrier peptide is about 4 to about 40 amino acids long. The length of the carrier peptide is not particularly limited and varies in different embodiments. In some embodiments, the carrier peptide comprises from about 4 to about 35 amino acid subunits. In other embodiments, the carrier peptide comprises from about 4 to about 30, from about 4 to about 25, from about 4 to about 20, from about 4 to about 15, from about 4 to about 10, or from about 4 to about 8 amino acid subunits. In various embodiments, the CPPs disclosed herein comprise an arginine-rich peptide as described further below. In some embodiments, the CPP or carrier peptide is at least about 4 amino acids long. In some embodiments, the CPP or carrier peptide is up to about 40 amino acids long. In some embodiments, the carrier peptide comprises 4 amino acids. In some embodiments, the carrier peptide comprises 5 amino acids. In some embodiments, the carrier peptide comprises 6 amino acids. In some embodiments, the carrier peptide comprises 7 amino acids. In some embodiments, the carrier peptide comprises 8 amino acids. In some embodiments, the carrier peptide comprises 9 amino acids. In some embodiments, the carrier peptide comprises 10 amino acids. In some embodiments, the carrier peptide comprises 11 amino acids. In some embodiments, the carrier peptide comprises 12 amino acids. In some embodiments, the carrier peptide comprises 13 amino acids. In some embodiments, the carrier peptide comprises 14 amino acids. In some embodiments, the carrier peptide comprises 15 amino acids. In some embodiments, the carrier peptide comprises 16 amino acids. In some embodiments, the carrier peptide comprises 17 amino acids. In some embodiments, the carrier peptide comprises 18 amino acids. In some embodiments, the carrier peptide comprises 19 amino acids. In some embodiments, the carrier peptide comprises 20 amino acids. In some embodiments, the carrier peptide comprises 21 amino acids. In some embodiments, the carrier peptide comprises 22 amino acids. In some embodiments, the carrier peptide comprises 23 amino acids. In some embodiments, the carrier peptide comprises 24 amino acids. In some embodiments, the carrier peptide comprises 25 amino acids. In some embodiments, the carrier peptide comprises 26 amino acids. In some embodiments, the carrier peptide comprises 27 amino acids. In some embodiments, the carrier peptide comprises 28 amino acids. In some embodiments, the carrier peptide comprises 29 amino acids. In some embodiments, the carrier peptide comprises 30 amino acids. In some embodiments, the carrier peptide comprises 31 amino acids. In some embodiments, the carrier peptide comprises 32 amino acids. In some embodiments, the carrier peptide comprises 33 amino acids. In some embodiments, the carrier peptide comprises 34 amino acids. In some embodiments, the carrier peptide comprises 35 amino acids. In some embodiments, the carrier peptide comprises 36 amino acids. In some embodiments, the carrier peptide comprises 37 amino acids. In some embodiments, the carrier peptide comprises 38 amino acids. In some embodiments, the carrier peptide comprises 39 amino acids. In some embodiments, the carrier peptide comprises 40 amino acids.

As used herein, a “peptide-conjugated phosphorodiamidate-linked morpholino oligomer” or “PPMO” refers to a PMO covalently linked to a peptide, such as a cell-penetrating peptide (CPP) or carrier peptide. The cell-penetrating peptide promotes the uptake of the PMO by cells, thereby delivering the PMO to the interior (cytoplasm) of the cells. Depending on its amino acid sequence, a CPP can be generally effective, or it can be specifically or selectively effective for PMO delivery to a particular type or particular types of cells. PMOs and CPPs are typically linked at their ends, e.g., the C-terminal end of the CPP can be linked to the 5′ end of the PMO, or 3′ end of the PMO can be linked to the N-terminal end of the CPP. PPMOs can include uncharged PMOs, charged (e.g., cationic) PMOs, and mixtures thereof. In an embodiment, the linking moiety of the conjugates described herein may be cleaved to release a PPMO.

The carrier peptide may be linked to the nucleic acid analog either directly or via an optional linker, e.g., one or more additional naturally occurring amino acids, e.g., cysteine (C), glycine (G), or proline (P), or additional amino acid analogs, e.g., 6-aminohexanoic acid (X) (also represented as Ahx or α) and beta-alanine (B) (also represented as β-Ala or β). Other linking moieties known in the art may also be employed.

An “amino acid subunit” is generally an α-amino acid residue (—CO—CHR—NH—); but may also be a β- or other amino acid residue (e.g., —CO—CH₂CHR—NH—), where R is an amino acid side chain.

The term “naturally occurring amino acid” refers to an amino acid present in proteins found in nature; examples include Alanine (A), Cysteine (C), Aspartic acid (D), Glutamic acid (E), Phenyalanine (F), Glycine (G), Histidine (H), Isoleucine (I), Lysine (K), Leucine (L). Methionine (M), Asparagine (N), Proline (P), Glutamine (Q), Arginine (R), Serine(S), Threonine (T), Valine (V), Tryptophan (W), and Tyrosine (Y). The term “non-natural amino acids” refers to those amino acids not present in proteins found in nature; examples include beta-alanine (β-Ala, B, or β) and 6-aminohexanoic acid (X, Ahx or α).

An agent is “actively taken up by mammalian cells” when the agent can enter the cell by a mechanism other than passive diffusion across the cell membrane. The agent may be transported, for example, by “active transport,” referring to transport of agents across a mammalian cell membrane by, e.g., an ATP-dependent transport mechanism, or by “facilitated transport,” referring to transport of antisense agents across the cell membrane by a transport mechanism that requires binding of the agent to a transport protein, which then facilitates passage of the bound agent across the membrane.

As used herein, an “effective amount” refers to any amount of a substance that is sufficient to achieve a desired biological result. A “therapeutically effective amount” refers to any amount of a substance that is sufficient to achieve a desired therapeutic result.

As used herein, a “subject” is a mammal, which can include a mouse, rat, hamster, guinea pig, rabbit, goat, sheep, cat, dog, pig, cow, horse, non-human primate such as a monkey, or a human. In certain embodiments, a subject is a human.

“Treatment” of an individual (e.g., a mammal, such as a human) or a cell is any type of intervention used to alter the natural course of the individual or cell. Treatment includes, but is not limited to, administration of a pharmaceutical composition, and may be performed either prophylactically or subsequent to the initiation of a pathologic event or contact with an etiologic agent.

### II. Peptide-Oligomers

Provided herein is an antisense oligomer, or a pharmaceutically acceptable salt thereof, comprising a non-natural chemical backbone and a targeting sequence of 13 to 30 bases in length that is complementary to a target region within a pre-mRNA of the human uromodulin (UMOD) gene (SEQ ID NO: 1). In some embodiments, the target region is an intron/exon junction or an exon internal region of the human UMOD gene pre-mRNA. In some embodiments, the target region is an intron/exon junction of the human UMOD gene pre-mRNA. In other embodiments, the target region is an exon internal region of the human UMOD gene pre-mRNA.

In an embodiment, the antisense oligomer, or a pharmaceutically acceptable salt thereof, is from 13 to 30 bases (subunits) in length. In some embodiments, the antisense oligomer, or a pharmaceutically acceptable salt thereof, is 13, 14, 15, 16, 17, 18, 19, 20, 21, 22, 23, 24, 25, 26, 27, 28, 29, or 30 bases in length. In an embodiment, the antisense oligomer, or a pharmaceutically acceptable salt thereof, is at least 13 bases in length. In an embodiment, the antisense oligomer, or a pharmaceutically acceptable salt thereof, is up to 30 bases in length.

In an embodiment, the antisense oligomer, or a pharmaceutically acceptable salt thereof, is 13-29 bases in length. In an embodiment, the antisense oligomer, or a pharmaceutically acceptable salt thereof, is 13-28 bases in length. In an embodiment, the antisense oligomer, or a pharmaceutically acceptable salt thereof, is 13-27 bases in length. In an embodiment, the antisense oligomer, or a pharmaceutically acceptable salt thereof, is 13-26 bases in length. In an embodiment, the antisense oligomer, or a pharmaceutically acceptable salt thereof, is 13-25 bases in length. In an embodiment, the antisense oligomer, or a pharmaceutically acceptable salt thereof, is 13-24 bases in length. In an embodiment, the antisense oligomer, or a pharmaceutically acceptable salt thereof, is 13-23 bases in length. In an embodiment, the antisense oligomer, or a pharmaceutically acceptable salt thereof, is 13-22 bases in length. In an embodiment, the antisense oligomer, or a pharmaceutically acceptable salt thereof, is 13-21 bases in length. In an embodiment, the antisense oligomer, or a pharmaceutically acceptable salt thereof, is 13-20 bases in length. In an embodiment, the antisense oligomer, or a pharmaceutically acceptable salt thereof, is 13-19 bases in length. In an embodiment, the antisense oligomer, or a pharmaceutically acceptable salt thereof, is 13-18 bases in length. In an embodiment, the antisense oligomer, or a pharmaceutically acceptable salt thereof, is 13-17 bases in length. In an embodiment, the antisense oligomer, or a pharmaceutically acceptable salt thereof, is 13-16 bases in length. In an embodiment, the antisense oligomer, or a pharmaceutically acceptable salt thereof, is 13-15 bases in length. In an embodiment, the antisense oligomer, or a pharmaceutically acceptable salt thereof, is 13-14 bases in length.

In another embodiment, the antisense oligomer, or a pharmaceutically acceptable salt thereof, is 14-30 bases in length. In an embodiment, the antisense oligomer, or a pharmaceutically acceptable salt thereof, is 14-29 bases in length. In an embodiment, the antisense oligomer, or a pharmaceutically acceptable salt thereof, is 14-28 bases in length. In an embodiment, the antisense oligomer, or a pharmaceutically acceptable salt thereof, is 14-27 bases in length. In an embodiment, the antisense oligomer, or a pharmaceutically acceptable salt thereof, is 14-26 bases in length. In an embodiment, the antisense oligomer, or a pharmaceutically acceptable salt thereof, is 14-25 bases in length. In an embodiment, the antisense oligomer, or a pharmaceutically acceptable salt thereof, is 14-24 bases in length. In an embodiment, the antisense oligomer, or a pharmaceutically acceptable salt thereof, is 14-23 bases in length. In an embodiment, the antisense oligomer, or a pharmaceutically acceptable salt thereof, is 14-22 bases in length. In an embodiment, the antisense oligomer, or a pharmaceutically acceptable salt thereof, is 14-21 bases in length. In an embodiment, the antisense oligomer, or a pharmaceutically acceptable salt thereof, is 14-20 bases in length. In an embodiment, the antisense oligomer, or a pharmaceutically acceptable salt thereof, is 14-19 bases in length. In an embodiment, the antisense oligomer, or a pharmaceutically acceptable salt thereof, is 14-18 bases in length. In an embodiment, the antisense oligomer, or a pharmaceutically acceptable salt thereof, is 14-17 bases in length. In an embodiment, the antisense oligomer, or a pharmaceutically acceptable salt thereof, is 14-16 bases in length. In an embodiment, the antisense oligomer, or a pharmaceutically acceptable salt thereof, is 14-15 bases in length.

In another embodiment, the antisense oligomer, or a pharmaceutically acceptable salt thereof, is 15-30 bases in length. In an embodiment, the antisense oligomer, or a pharmaceutically acceptable salt thereof, is 15-29 bases in length. In an embodiment, the antisense oligomer, or a pharmaceutically acceptable salt thereof, is 15-28 bases in length. In an embodiment, the antisense oligomer, or a pharmaceutically acceptable salt thereof, is 15-27 bases in length. In an embodiment, the antisense oligomer, or a pharmaceutically acceptable salt thereof, is 15-26 bases in length. In an embodiment, the antisense oligomer, or a pharmaceutically acceptable salt thereof, is 15-25 bases in length. In an embodiment, the antisense oligomer, or a pharmaceutically acceptable salt thereof, is 15-24 bases in length. In an embodiment, the antisense oligomer, or a pharmaceutically acceptable salt thereof, is 15-23 bases in length. In an embodiment, the antisense oligomer, or a pharmaceutically acceptable salt thereof, is 15-22 bases in length. In an embodiment, the antisense oligomer, or a pharmaceutically acceptable salt thereof, is 15-21 bases in length. In an embodiment, the antisense oligomer, or a pharmaceutically acceptable salt thereof, is 15-20 bases in length. In an embodiment, the antisense oligomer, or a pharmaceutically acceptable salt thereof, is 15-19 bases in length. In an embodiment, the antisense oligomer, or a pharmaceutically acceptable salt thereof, is 15-18 bases in length. In an embodiment, the antisense oligomer, or a pharmaceutically acceptable salt thereof, is 15-17 bases in length. In an embodiment, the antisense oligomer, or a pharmaceutically acceptable salt thereof, is 15-16 bases in length.

In another embodiment, the antisense oligomer, or a pharmaceutically acceptable salt thereof, is 16-30 bases in length. In an embodiment, the antisense oligomer, or a pharmaceutically acceptable salt thereof, is 16-29 bases in length. In an embodiment, the antisense oligomer, or a pharmaceutically acceptable salt thereof, is 16-28 bases in length. In an embodiment, the antisense oligomer, or a pharmaceutically acceptable salt thereof, is 16-27 bases in length. In an embodiment, the antisense oligomer, or a pharmaceutically acceptable salt thereof, is 16-26 bases in length. In an embodiment, the antisense oligomer, or a pharmaceutically acceptable salt thereof, is 16-25 bases in length. In an embodiment, the antisense oligomer, or a pharmaceutically acceptable salt thereof, is 16-24 bases in length. In an embodiment, the antisense oligomer, or a pharmaceutically acceptable salt thereof, is 16-23 bases in length. In an embodiment, the antisense oligomer, or a pharmaceutically acceptable salt thereof, is 16-22 bases in length. In an embodiment, the antisense oligomer, or a pharmaceutically acceptable salt thereof, is 16-21 bases in length. In an embodiment, the antisense oligomer, or a pharmaceutically acceptable salt thereof, is 16-20 bases in length. In an embodiment, the antisense oligomer, or a pharmaceutically acceptable salt thereof, is 16-19 bases in length. In an embodiment, the antisense oligomer, or a pharmaceutically acceptable salt thereof, is 16-18 bases in length. In an embodiment, the antisense oligomer, or a pharmaceutically acceptable salt thereof, is 16-17 bases in length.

In another embodiment, the antisense oligomer, or a pharmaceutically acceptable salt thereof, is 17-30 bases in length. In an embodiment, the antisense oligomer, or a pharmaceutically acceptable salt thereof, is 17-29 bases in length. In an embodiment, the antisense oligomer, or a pharmaceutically acceptable salt thereof, is 17-28 bases in length. In an embodiment, the antisense oligomer, or a pharmaceutically acceptable salt thereof, is 17-27 bases in length. In an embodiment, the antisense oligomer, or a pharmaceutically acceptable salt thereof, is 17-26 bases in length. In an embodiment, the antisense oligomer, or a pharmaceutically acceptable salt thereof, is 17-25 bases in length. In an embodiment, the antisense oligomer, or a pharmaceutically acceptable salt thereof, is 17-24 bases in length. In an embodiment, the antisense oligomer, or a pharmaceutically acceptable salt thereof, is 17-23 bases in length. In an embodiment, the antisense oligomer, or a pharmaceutically acceptable salt thereof, is 17-22 bases in length. In an embodiment, the antisense oligomer, or a pharmaceutically acceptable salt thereof, is 17-21 bases in length. In an embodiment, the antisense oligomer, or a pharmaceutically acceptable salt thereof, is 17-20 bases in length. In an embodiment, the antisense oligomer, or a pharmaceutically acceptable salt thereof, is 17-19 bases in length. In an embodiment, the antisense oligomer, or a pharmaceutically acceptable salt thereof, is 17-18 bases in length.

In another embodiment, the antisense oligomer, or a pharmaceutically acceptable salt thereof, is 18-30 bases in length. In an embodiment, the antisense oligomer, or a pharmaceutically acceptable salt thereof, is 18-29 bases in length. In an embodiment, the antisense oligomer, or a pharmaceutically acceptable salt thereof, is 18-28 bases in length. In an embodiment, the antisense oligomer, or a pharmaceutically acceptable salt thereof, is 18-27 bases in length. In an embodiment, the antisense oligomer, or a pharmaceutically acceptable salt thereof, is 18-26 bases in length. In an embodiment, the antisense oligomer, or a pharmaceutically acceptable salt thereof, is 18-25 bases in length. In an embodiment, the antisense oligomer, or a pharmaceutically acceptable salt thereof, is 18-24 bases in length. In an embodiment, the antisense oligomer, or a pharmaceutically acceptable salt thereof, is 18-23 bases in length. In an embodiment, the antisense oligomer, or a pharmaceutically acceptable salt thereof, is 18-22 bases in length. In an embodiment, the antisense oligomer, or a pharmaceutically acceptable salt thereof, is 18-21 bases in length. In an embodiment, the antisense oligomer, or a pharmaceutically acceptable salt thereof, is 18-20 bases in length. In an embodiment, the antisense oligomer, or a pharmaceutically acceptable salt thereof, is 18-19 bases in length.

In another embodiment, the antisense oligomer, or a pharmaceutically acceptable salt thereof, is 19-30 bases in length. In an embodiment, the antisense oligomer, or a pharmaceutically acceptable salt thereof, is 19-29 bases in length. In an embodiment, the antisense oligomer, or a pharmaceutically acceptable salt thereof, is 19-28 bases in length. In an embodiment, the antisense oligomer, or a pharmaceutically acceptable salt thereof, is 19-27 bases in length. In an embodiment, the antisense oligomer, or a pharmaceutically acceptable salt thereof, is 19-26 bases in length. In an embodiment, the antisense oligomer, or a pharmaceutically acceptable salt thereof, is 19-25 bases in length. In an embodiment, the antisense oligomer, or a pharmaceutically acceptable salt thereof, is 19-24 bases in length. In an embodiment, the antisense oligomer, or a pharmaceutically acceptable salt thereof, is 19-23 bases in length. In an embodiment, the antisense oligomer, or a pharmaceutically acceptable salt thereof, is 19-22 bases in length. In an embodiment, the antisense oligomer, or a pharmaceutically acceptable salt thereof, is 19-21 bases in length. In an embodiment, the antisense oligomer, or a pharmaceutically acceptable salt thereof, is 19-20 bases in length.

In another embodiment, the antisense oligomer, or a pharmaceutically acceptable salt thereof, is 20-30 bases in length. In an embodiment, the antisense oligomer, or a pharmaceutically acceptable salt thereof, is 20-29 bases in length. In an embodiment, the antisense oligomer, or a pharmaceutically acceptable salt thereof, is 20-28 bases in length. In an embodiment, the antisense oligomer, or a pharmaceutically acceptable salt thereof, is 20-27 bases in length. In an embodiment, the antisense oligomer, or a pharmaceutically acceptable salt thereof, is 20-26 bases in length. In an embodiment, the antisense oligomer, or a pharmaceutically acceptable salt thereof, is 20-25 bases in length. In an embodiment, the antisense oligomer, or a pharmaceutically acceptable salt thereof, is 20-24 bases in length. In an embodiment, the antisense oligomer, or a pharmaceutically acceptable salt thereof, is 20-23 bases in length. In an embodiment, the antisense oligomer, or a pharmaceutically acceptable salt thereof, is 20-22 bases in length. In an embodiment, the antisense oligomer, or a pharmaceutically acceptable salt thereof, is 20-21 bases in length.

In another embodiment, the antisense oligomer, or a pharmaceutically acceptable salt thereof, is 21-30 bases in length. In an embodiment, the antisense oligomer, or a pharmaceutically acceptable salt thereof, is 21-29 bases in length. In an embodiment, the antisense oligomer, or a pharmaceutically acceptable salt thereof, is 21-28 bases in length. In an embodiment, the antisense oligomer, or a pharmaceutically acceptable salt thereof, is 21-27 bases in length. In an embodiment, the antisense oligomer, or a pharmaceutically acceptable salt thereof, is 21-26 bases in length. In an embodiment, the antisense oligomer, or a pharmaceutically acceptable salt thereof, is 21-25 bases in length. In an embodiment, the antisense oligomer, or a pharmaceutically acceptable salt thereof, is 21-24 bases in length. In an embodiment, the antisense oligomer, or a pharmaceutically acceptable salt thereof, is 21-23 bases in length. In an embodiment, the antisense oligomer, or a pharmaceutically acceptable salt thereof, is 21-22 bases in length.

In another embodiment, the antisense oligomer, or a pharmaceutically acceptable salt thereof, is 22-30 bases in length. In an embodiment, the antisense oligomer, or a pharmaceutically acceptable salt thereof, is 22-29 bases in length. In an embodiment, the antisense oligomer, or a pharmaceutically acceptable salt thereof, is 22-28 bases in length. In an embodiment, the antisense oligomer, or a pharmaceutically acceptable salt thereof, is 22-27 bases in length. In an embodiment, the antisense oligomer, or a pharmaceutically acceptable salt thereof, is 22-26 bases in length. In an embodiment, the antisense oligomer, or a pharmaceutically acceptable salt thereof, is 22-25 bases in length. In an embodiment, the antisense oligomer, or a pharmaceutically acceptable salt thereof, is 22-24 bases in length. In an embodiment, the antisense oligomer, or a pharmaceutically acceptable salt thereof, is 22-23 bases in length.

In another embodiment, the antisense oligomer, or a pharmaceutically acceptable salt thereof, is 23-30 bases in length. In an embodiment, the antisense oligomer, or a pharmaceutically acceptable salt thereof, is 23-29 bases in length. In an embodiment, the antisense oligomer, or a pharmaceutically acceptable salt thereof, is 23-28 bases in length. In an embodiment, the antisense oligomer, or a pharmaceutically acceptable salt thereof, is 23-27 bases in length. In an embodiment, the antisense oligomer, or a pharmaceutically acceptable salt thereof, is 23-26 bases in length. In an embodiment, the antisense oligomer, or a pharmaceutically acceptable salt thereof, is 23-25 bases in length. In an embodiment, the antisense oligomer, or a pharmaceutically acceptable salt thereof, is 23-24 bases in length.

In another embodiment, the antisense oligomer, or a pharmaceutically acceptable salt thereof, is 24-30 bases in length. In an embodiment, the antisense oligomer, or a pharmaceutically acceptable salt thereof, is 24-29 bases in length. In an embodiment, the antisense oligomer, or a pharmaceutically acceptable salt thereof, is 24-28 bases in length. In an embodiment, the antisense oligomer, or a pharmaceutically acceptable salt thereof, is 24-27 bases in length. In an embodiment, the antisense oligomer, or a pharmaceutically acceptable salt thereof, is 24-26 bases in length. In an embodiment, the antisense oligomer, or a pharmaceutically acceptable salt thereof, is 24-25 bases in length.

In another embodiment, the antisense oligomer, or a pharmaceutically acceptable salt thereof, is 25-30 bases in length. In an embodiment, the antisense oligomer, or a pharmaceutically acceptable salt thereof, is 25-29 bases in length. In an embodiment, the antisense oligomer, or a pharmaceutically acceptable salt thereof, is 25-28 bases in length. In an embodiment, the antisense oligomer, or a pharmaceutically acceptable salt thereof, is 25-27 bases in length. In an embodiment, the antisense oligomer, or a pharmaceutically acceptable salt thereof, is 25-26 bases in length.

In another embodiment, the antisense oligomer, or a pharmaceutically acceptable salt thereof, is 26-30 bases in length. In an embodiment, the antisense oligomer, or a pharmaceutically acceptable salt thereof, is 26-29 bases in length. In an embodiment, the antisense oligomer, or a pharmaceutically acceptable salt thereof, is 26-28 bases in length. In an embodiment, the antisense oligomer, or a pharmaceutically acceptable salt thereof, is 26-27 bases in length.

In another embodiment, the antisense oligomer, or a pharmaceutically acceptable salt thereof, is 27-30 bases in length. In an embodiment, the antisense oligomer, or a pharmaceutically acceptable salt thereof, is 27-29 bases in length. In an embodiment, the antisense oligomer, or a pharmaceutically acceptable salt thereof, is 27-28 bases in length.

In another embodiment, the antisense oligomer, or a pharmaceutically acceptable salt thereof, is 28-30 bases in length. In an embodiment, the antisense oligomer, or a pharmaceutically acceptable salt thereof, is 28-29 bases in length. In another embodiment, the antisense oligomer, or a pharmaceutically acceptable salt thereof, is 28-30 bases in length. In an embodiment, the antisense oligomer, or a pharmaceutically acceptable salt thereof, is 29-30 bases in length.

In an embodiment, the antisense oligomer, or a pharmaceutically acceptable salt thereof, is 13 bases in length. In an embodiment, the antisense oligomer, or a pharmaceutically acceptable salt thereof, is 14 bases in length. In an embodiment, the antisense oligomer, or a pharmaceutically acceptable salt thereof, is 15 bases in length. In an embodiment, the antisense oligomer, or a pharmaceutically acceptable salt thereof, is 16 bases in length. In an embodiment, the antisense oligomer, or a pharmaceutically acceptable salt thereof, is 17 bases in length. In an embodiment, the antisense oligomer, or a pharmaceutically acceptable salt thereof, is 18 bases in length. In an embodiment, the antisense oligomer, or a pharmaceutically acceptable salt thereof, is 19 bases in length. In an embodiment, the antisense oligomer, or a pharmaceutically acceptable salt thereof, is 20 bases in length. In an embodiment, the antisense oligomer, or a pharmaceutically acceptable salt thereof, is 21 bases in length. In an embodiment, the antisense oligomer, or a pharmaceutically acceptable salt thereof, is 22 bases in length. In an embodiment, the antisense oligomer, or a pharmaceutically acceptable salt thereof, is 23 bases in length. In an embodiment, the antisense oligomer, or a pharmaceutically acceptable salt thereof, is 24 bases in length. In an embodiment, the antisense oligomer, or a pharmaceutically acceptable salt thereof, is 25 bases in length. In an embodiment, the antisense oligomer, or a pharmaceutically acceptable salt thereof, is 26 bases in length. In an embodiment, the antisense oligomer, or a pharmaceutically acceptable salt thereof, is 27 bases in length. In an embodiment, the antisense oligomer, or a pharmaceutically acceptable salt thereof, is 28 bases in length. In an embodiment, the antisense oligomer, or a pharmaceutically acceptable salt thereof, is 29 bases in length. In an embodiment, the antisense oligomer, or a pharmaceutically acceptable salt thereof, is 30 bases in length.

In an embodiment, the antisense oligomer comprises a targeting sequence complementary to a target region within a pre-mRNA of the human UMOD gene. In certain embodiments, the target region is an intron/exon junction or an exon internal region of exon 2 (SEQ ID NO: 2), exon 5 (SEQ ID NO: 3), exon 6 (SEQ ID NO: 4), exon 8 (SEQ ID NO: 5) or exon 9 (SEQ ID NO: 6).

In some embodiments, the antisense oligomer, or a pharmaceutically acceptable salt thereof, has a target region selected from UMOD H2A(−25-1), UMOD H2A(−18+2), UMOD H2A(−17+3), UMOD H2A(−16+4), UMOD H2A(−15+10), UMOD H2A(+1+25), UMOD H2A(+26+50), UMOD H2A(+51+75), UMOD H2A(+85+104), UMOD H2A(+86+105), UMOD H2A(+95+119), UMOD H2A(+96+115), UMOD H2A(+98+117), UMOD H2A(+101+125), UMOD H2A(+108+127), UMOD H2A(+109+128), UMOD H2A(+110+129), UMOD H2A(+113+132), UMOD H2A(+114+133), UMOD H2A(+118+137), UMOD H2A(+126+150), UMOD H2A(+151+175), UMOD H2D (+15-10), UMOD H5A (−15+5), UMOD H5A (−14+6), UMOD H5A (−11+9), UMOD H5A (−10+10), UMOD H5A (−9+11), UMOD H5A(+50+67), UMOD H5A(+51+75), UMOD H5A(+52+69), UMOD H5A(+76+100), UMOD H5A(+78+95), UMOD H5A(+80+97), UMOD H5A(+82+99), UMOD H5A(+85+102), UMOD H5A(+86+103), UMOD H5A(+91+108), UMOD H5A(+126+150), UMOD H5A(+152+171), UMOD H5A(+153+172), UMOD H5A(+154+173), UMOD H5D (+18-2), UMOD H5D (+17-3), UMOD H5D (+16-4), UMOD H5D (+14-6), UMOD H5D (+13-7), UMOD H5D (+12-8), UMOD H5D (+11-9), UMOD H5D (+10-10), UMOD H6A(+1+25), UMOD H6A(+26+50), UMOD H6A(+48+67), UMOD H6A(+49+68), UMOD H6A(+50+69), UMOD H6A(+58+77), UMOD H6A(+59+78), UMOD H6A(+60+79), UMOD H6A(+76+100), UMOD H6A(+79+98), UMOD H6A(+101+125), UMOD H6A(+101+120), UMOD H6A(+110+129), UMOD H6A(+111+130), UMOD H6A(+112+131), UMOD H6A(+113+132), UMOD H6A(+119+138), UMOD H6A(+120+139), UMOD H6A(+121+140), UMOD H6A(+122+141), UMOD H6A(+123+142), UMOD H6A(+124+143), UMOD H6A(+130+149), UMOD H6D (+24-1), UMOD H6D (+15-5), UMOD H6D (+14-6), UMOD H8A (−2+23), UMOD H8A(+26+50), UMOD H8A(+51+75), UMOD H8A(+60+79), UMOD H8A(+61+80), UMOD H8A(+62+81), UMOD H8A(+63+82), UMOD H8A(+68+87), UMOD H8A(+69+88), UMOD H8A(+70+89), UMOD H8A(+76+95), UMOD H8A(+76+100), UMOD H8A(+77+96), UMOD H8A(+78+97), UMOD H8A(+79+98), UMOD H8A(+80+99), UMOD H8A(+81+100), UMOD H8A(+82+101), UMOD H8A(+83+102), UMOD H8A(+86+105), UMOD H8A(+94+113), UMOD H8A(+95+114), UMOD H8A(+96+115), UMOD H8A(+101+125), UMOD H8A(+102+121), UMOD H8A(+103+122), UMOD H8A(+104+123), UMOD H8A(+105+124), UMOD H8A(+120+139), UMOD H8A(+121+140), UMOD H8A(+122+141), UMOD H8A(+126+150), UMOD H8A(+128+147), UMOD H8A(+129+148), UMOD H8A(+133+152), UMOD H8A(+134+153), UMOD H8A(+139+158), UMOD H8D (+19-1), UMOD H8D (+12-13), UMOD H9A (−5+20), UMOD H9A(+1+25), UMOD H9A(+51+75), and UMOD H9D (+7-18).

In some embodiments, target region is UMOD H2A(−25−1). In some embodiments, the target region is UMOD H2A(−18+2). In some embodiments, the target region is UMOD H2A(−17+3). In some embodiments, the target region is UMOD H2A(−16+4). In some embodiments, the target region is UMOD H2A(−15+10). In some embodiments, the target region is UMOD H2A(+1+25). In some embodiments, the target region is UMOD H2A(+26+50). In some embodiments, the target region is UMOD H2A(+51+75). In some embodiments, the target region is UMOD H2A(+85+104). In some embodiments, the target region is UMOD H2A(+86+105). In some embodiments, the target region is UMOD H2A(+95+119). UMOD H2A(+96+115). In some embodiments, the target region is UMOD In some embodiments, the target region is H2A(+98+117). In some embodiments, the target region is UMOD H2A(+101+125). In some embodiments, the target region is UMOD H2A(+108+127). In some embodiments, the target region is UMOD H2A(+109+128). In some embodiments, the target region is UMOD H2A(+110+129). In some embodiments, the target region is UMOD H2A(+113+132). In some embodiments, the target region is UMOD H2A(+114+133). In some embodiments, the target region is UMOD H2A(+118+137). In some embodiments, the target region is UMOD H2A(+126+150). In some embodiments, the target region is UMOD H2A(+151+175). In some embodiments, the target region is UMOD H2D (+15-10). In some embodiments, the target region is UMOD H5A (−15+5). In some embodiments, the target region is UMOD H5A (−14+6). In some embodiments, the target region is UMOD H5A (−11+9). In some embodiments, the target region is UMOD H5A (−10+10). In some embodiments, the target region is UMOD H5A (−9+11). In some embodiments, the target region is UMOD H5A(+50+67). In some embodiments, the target region is UMOD H5A(+51+75). In some embodiments, the target region is UMOD H5A(+52+69). In some embodiments, the target region is UMOD H5A(+76+100). In some embodiments, the target region is UMOD H5A(+78+95). In some embodiments, the target region is UMOD H5A(+80+97). In some embodiments, the target region is UMOD H5A(+82+99). In some embodiments, the target region is UMOD H5A(+85+102). In some embodiments, the target region is UMOD H5A(+86+103). In some embodiments, the target region is UMOD H5A(+91+108). In some embodiments, the target region is UMOD H5A(+126+150). In some embodiments, the target region is UMOD H5A(+152+171). In some embodiments, the target region is UMOD H5A(+153+172). In some embodiments, the target region is UMOD H5A(+154+173). In some embodiments, the target region is UMOD H5D (+18-2). In some embodiments, the target region is UMOD H5D (+17-3). In some embodiments, the target region is UMOD H5D (+16-4). In some embodiments, the target region is UMOD H5D (+14-6). In some embodiments, the target region is UMOD H5D (+13-7). In some embodiments, the target region is UMOD H5D (+12-8). UMOD H5D (+11-9). In some embodiments, the target region is UMOD H5D (+10-10). In some embodiments, the target region is UMOD H6A(+1+25). In some embodiments, the target region is UMOD H6A(+26+50). In some embodiments, the target region is UMOD H6A(+48+67). In some embodiments, the target region is UMOD H6A(+49+68). In some embodiments, the target region is UMOD H6A(+50+69). In some embodiments, the target region is UMOD H6A(+58+77). In some embodiments, the target region is UMOD H6A(+59+78). In some embodiments, the target region is UMOD H6A(+60+79). In some embodiments, the target region is UMOD H6A(+76+100). In some embodiments, the target region is UMOD H6A(+79+98). In some embodiments, the target region is UMOD H6A(+101+125). In some embodiments, the target region is UMOD H6A(+101+120). In some embodiments, the target region is UMOD H6A(+110+129). In some embodiments, the target region is UMOD H6A(+111+130). In some embodiments, the target region is UMOD H6A(+112+131). In some embodiments, the target region is UMOD H6A(+113+132). In some embodiments, the target region is UMOD H6A(+119+138). In some embodiments, the target region is UMOD H6A(+120+139). In some embodiments, the target region is UMOD H6A(+121+140). In some embodiments, the target region is UMOD H6A(+122+141). In some embodiments, the target region is UMOD H6A(+123+142). In some embodiments, the target region is UMOD H6A(+124+143). In some embodiments, the target region is UMOD H6A(+130+149). In some embodiments, the target region is UMOD H6D (+24-1). In some embodiments, the target region is UMOD H6D (+15-5). In some embodiments, the target region is UMOD H6D (+14−6). In some embodiments, the target region is UMOD H8A (−2+23). In some embodiments, the target region is UMOD H8A(+26+50). In some embodiments, the target region is UMOD H8A(+51+75). In some embodiments, the target region is UMOD H8A(+60+79). In some embodiments, the target region is UMOD H8A(+61+80). In some embodiments, the target region is UMOD H8A(+62+81). In some embodiments, the target region is UMOD H8A(+63+82). In some embodiments, the target region is UMOD H8A(+68+87). In some embodiments, the target region is UMOD H8A(+69+88). In some embodiments, the target region is UMOD H8A(+70+89). In some embodiments, the target region is UMOD H8A(+76+95). In some embodiments, the target region is UMOD H8A(+76+100). In some embodiments, the target region is UMOD H8A(+77+96). In some embodiments, the target region is UMOD H8A(+78+97). In some embodiments, the target region is UMOD H8A(+79+98). In some embodiments, the target region is UMOD H8A(+80+99). In some embodiments, the target region is UMOD H8A(+81+100). In some embodiments, the target region is UMOD H8A(+82+101). In some embodiments, the target region is UMOD H8A(+83+102). In some embodiments, the target region is UMOD H8A(+86+105). In some embodiments, the target region is UMOD H8A(+94+113). In some embodiments, the target region is UMOD H8A(+95+114). In some embodiments, the target region is UMOD H8A(+96+115). In some embodiments, the target region is UMOD H8A(+101+125). In some embodiments, the target region is UMOD H8A(+102+121). In some embodiments, the target region is UMOD H8A(+103+122). In some embodiments, the target region is UMOD H8A(+104+123). In some embodiments, the target region is UMOD H8A(+105+124). In some embodiments, the target region is UMOD H8A(+120+139). In some embodiments, the target region is UMOD H8A(+121+140). In some embodiments, the target region is UMOD H8A(+122+141). In some embodiments, the target region is UMOD H8A(+126+150). In some embodiments, the target region is UMOD H8A(+128+147). In some embodiments, the target region is UMOD H8A(+129+148). In some embodiments, the target region is UMOD H8A(+133+152). In some embodiments, the target region is UMOD H8A(+134+153). In some embodiments, the target region is UMOD H8A(+139+158). In some embodiments, the target region is UMOD H8D (+19-1). In some embodiments, the target region is UMOD H8D (+12-13). In some embodiments, the target region is UMOD H9A (−5+20). In some embodiments, the target region is UMOD H9A(+1+25). In some embodiments, the target region is UMOD H9A(+51+75). In some embodiments, the target region is UMOD H9D (+7-18).

In some embodiments, the antisense oligomer, or a pharmaceutically acceptable salt thereof, has a targeting sequence comprising a sequence selected from:

SEQ ID NO: 7
(CTGTGAACAGAGATGGATGGGACAA);
SEQ ID NO: 8
(TCCTGTGAACAGAGATGGAT);
SEQ ID NO: 9
(GTCCTGTGAACAGAGATGGA);
SEQ ID NO: 10
(TGTCCTGTGAACAGAGATGG);
SEQ ID NO: 11
(GTCTGGTGTCCTGTGAACAGAGATG);
SEQ ID NO: 12
(CTCTCTGTCTCTGATGTCTGGTGTC);
SEQ ID NO: 13
(AGACGGGTTGGCCCTTTGAATTTTT);
SEQ ID NO: 14
(TCTAGATAGCACCTGCCCAAAGGAA);
SEQ ID NO: 15
(ATCCTTTCTGCTCTTCCCGC)
SEQ ID NO: 16
(CATCCTTTCTGCTCTTCCCG);
SEQ ID NO: 17
(AGAGATGGCTGCCCCATCCTTTCTG);
SEQ ID NO: 18
(ATGGCTGCCCCATCCTTTCT);
SEQ ID NO: 19
(AGATGGCTGCCCCATCCTTT);
SEQ ID NO: 20
(CAAGTCAGAGATGGCTGCCCCATCC);
SEQ ID NO: 21
(TCCAAGTCAGAGATGGCTGC);
SEQ ID NO: 22
(ATCCAAGTCAGAGATGGCTG);
SEQ ID NO: 23
(CATCCAAGTCAGAGATGGCT);
SEQ ID NO: 24
(CAGCATCCAAGTCAGAGATG);
SEQ ID NO: 25
(TCAGCATCCAAGTCAGAGAT);
SEQ ID NO: 26
(ACCATCAGCATCCAAGTCAG);
SEQ ID NO: 27
(AGAGGCCACCACCACCATCAGCATC);
SEQ ID NO: 28
(CAGTGGCTGCAGTTGTGATGAACCA);
SEQ ID NO: 29
(TTTCACTTACTTGCTTCTGAGGTGT);
SEQ ID NO: 30
(GATATCTGAAACAGGTTAGG);
SEQ ID NO: 31
(AGATATCTGAAACAGGTTAG);
SEQ ID NO: 32
(GGGAGATATCTGAAACAGGT);
SEQ ID NO: 33
(AGGGAGATATCTGAAACAGG);
SEQ ID NO: 34
(CACTTGCCCAGCGACACC);
SEQ ID NO: 35
(TCAGCTGGCACTTGCCCAGCGACAC);
SEQ ID NO: 36
(GGCACTTGCCCAGCGACA);
SEQ ID NO: 37
(AAGACCTTGTCGAAGCCCAGACTCT);
SEQ ID NO: 38
(CTTGTCGAAGCCCAGACT);
SEQ ID NO: 39
(ACCTTGTCGAAGCCCAGA);
SEQ ID NO: 40
(AGACCTTGTCGAAGCCCA);
SEQ ID NO: 41
(TGAAGACCTTGTCGAAGC);
SEQ ID NO: 42
(ATGAAGACCTTGTCGAAG);
SEQ ID NO: 43
(GGTACATGAAGACCTTGT);
SEQ ID NO: 44
(GGTTGTCTCTGTCATTGAAGCCCGA);
SEQ ID NO: 45
(TCACTACAGACACCCAGTCC);
SEQ ID NO: 46
(GTCACTACAGACACCCAGTC);
SEQ ID NO: 47
(GGTCACTACAGACACCCAGT);
SEQ ID NO: 48
(ACCGTCAACACTGTCCCACA);
SEQ ID NO: 49
(TACCGTCAACACTGTCCCAC);
SEQ ID NO: 50
(GTACCGTCAACACTGTCCCA);
SEQ ID NO: 51
(ACGTACCGTCAACACTGTCC);
SEQ ID NO: 52
(GACGTACCGTCAACACTGTC);
SEQ ID NO: 53
(GGACGTACCGTCAACACTGT);
SEQ ID NO: 54
(AGGACGTACCGTCAACACTG);
SEQ ID NO: 55
(CAGGACGTACCGTCAACACT);
SEQ ID NO: 56
(TGTAAGTGGCATGGGTTTCATTCCT);
SEQ ID NO: 57
(TCATCTGCCAGGTAGAGGGTGTTGC);
SEQ ID NO: 58
(GGTCACGGATGATGATCTCA);
SEQ ID NO: 59
(AGGTCACGGATGATGATCTC);
SEQ ID NO: 60
(GAGGTCACGGATGATGATCT);
SEQ ID NO: 61
(TTGATGTTGAGGTCACGGAT);
SEQ ID NO: 62
(TTTGATGTTGAGGTCACGGA);
SEQ ID NO: 63
(TTTTGATGTTGAGGTCACGG);
SEQ ID NO: 64
(GGTAGGAGCATGCAAAGTTGATTTT);
SEQ ID NO: 65
(TAGGAGCATGCAAAGTTGAT);
SEQ ID NO: 66
(TTCAGGCTGACTTTCATGTCCAGGG);
SEQ ID NO: 67
(GCTGACTTTCATGTCCAGGG);
SEQ ID NO: 68
(GGTCTTCAGGCTGACTTTCA);
SEQ ID NO: 69
(CGGTCTTCAGGCTGACTTTC);
SEQ ID NO: 70
(GCGGTCTTCAGGCTGACTTT);
SEQ ID NO: 71
(GGCGGTCTTCAGGCTGACTT);
SEQ ID NO: 72
(CTGTAGGGCGGTCTTCAGGC);
SEQ ID NO: 73
(GCTGTAGGGCGGTCTTCAGG);
SEQ ID NO: 74
(GGCTGTAGGGCGGTCTTCAG);
SEQ ID NO: 75
(TGGCTGTAGGGCGGTCTTCA);
SEQ ID NO: 76
(TTGGCTGTAGGGCGGTCTTC);
SEQ ID NO: 77
(ATTGGCTGTAGGGCGGTCTT);
SEQ ID NO: 78
(CTGACCATTGGCTGTAGGGC);
SEQ ID NO: 79
(CCTGACCATTGGCTGTAGGGCGGTC);
SEQ ID NO: 80
(CACACCTGACCATTGGCTGT);
SEQ ID NO: 81
(CCACACCTGACCATTGGCTG);
SEQ ID NO: 82
(TTGAGTCTCTAGTGTGTGGGCATCT);
SEQ ID NO: 83
(ACTCCCCATTCTCCACCACTTGGAT);
SEQ ID NO: 84
(TGGACGGAAAATCGGCCCTGGGAGG);
SEQ ID NO: 85
(CATCTGGACGGAAAATCGGC);
SEQ ID NO: 86
(ACATCTGGACGGAAAATCGG);
SEQ ID NO: 87
(AACATCTGGACGGAAAATCG);
SEQ ID NO: 88
(GAACATCTGGACGGAAAATC);
SEQ ID NO: 89
(AACCGGAACATCTGGACGGA);
SEQ ID NO: 90
(AAACCGGAACATCTGGACGG);
SEQ ID NO: 91
(CAAACCGGAACATCTGGACG);
SEQ ID NO: 92
(TTCCAGCAAACCGGAACATC);
SEQ ID NO: 93
(ATAGTTTCCAGCAAACCGGAACATC);
SEQ ID NO: 94
(TTTCCAGCAAACCGGAACAT);
SEQ ID NO: 95
(GTTTCCAGCAAACCGGAACA);
SEQ ID NO: 96
(AGTTTCCAGCAAACCGGAAC);
SEQ ID NO: 97
(TAGTTTCCAGCAAACCGGAA);
SEQ ID NO: 98
(ATAGTTTCCAGCAAACCGGA);
SEQ ID NO: 99
(CATAGTTTCCAGCAAACCGG);
SEQ ID NO: 100
(TCATAGTTTCCAGCAAACCG);
SEQ ID NO: 101
(AGGTCATAGTTTCCAGCAAA);
SEQ ID NO: 102
(GGTAGACTAGGTCATAGTTT);
SEQ ID NO: 103
(AGGTAGACTAGGTCATAGTT);
SEQ ID NO: 104
(CAGGTAGACTAGGTCATAGT);
SEQ ID NO: 105
(CTTCACAGTGCAGGTAGACTAGGTC);
SEQ ID NO: 106
(ACAGTGCAGGTAGACTAGGT);
SEQ ID NO: 107
(CACAGTGCAGGTAGACTAGG);
SEQ ID NO: 108
(TCACAGTGCAGGTAGACTAG);
SEQ ID NO: 109
(TTCACAGTGCAGGTAGACTA);
SEQ ID NO: 110
(GTCACAGAGATAGACTTCAC);
SEQ ID NO: 111
(TGTCACAGAGATAGACTTCA);
SEQ ID NO: 112
(GTGTCACAGAGATAGACTTC);
SEQ ID NO: 113
(TCATTCATGGTGTCACAGAGATAGA);
SEQ ID NO: 114
(TTCATGGTGTCACAGAGATA);
SEQ ID NO: 115
(ATTCATGGTGTCACAGAGAT);
SEQ ID NO: 116
(TTTCATTCATGGTGTCACAG);
SEQ ID NO: 117
(TTTTCATTCATGGTGTCACA);
SEQ ID NO: 118
(TGCACTTTTCATTCATGGTG);
SEQ ID NO: 119
(AGGCTTGCACTTTTCATTCA);
SEQ ID NO: 120
(GAGTCAACTCACAGGCTTGCACTTT);
SEQ ID NO: 121
(AATCTGGTCCCAGAGCAGGTCTACA);
SEQ ID NO: 122
(TTCGGAATCTGGTCCCAGAGCAGGT);
SEQ ID NO: 123
(TGTGATGGGACCCAAGTTCAGGACA);
SEQ ID NO: 124
(AGCGAGTGGCTCTCTTACCTTTCCG);
and
SEQ ID NO: 125
(GAGGGAGATATCTGAAACAG).

In some embodiments, the targeting sequence is SEQ ID NO: 7. In some embodiments, the targeting sequence is SEQ ID NO: 8. In some embodiments, the targeting sequence is SEQ ID NO: 9. In some embodiments, the targeting sequence is SEQ ID NO: 10. In some embodiments, the targeting sequence is SEQ ID NO: 11. In some embodiments, the targeting sequence is SEQ ID NO: 12. In some embodiments, the targeting sequence is SEQ ID NO: 13. In some embodiments, the targeting sequence is SEQ ID NO: 14. In some embodiments, the targeting sequence is SEQ ID NO: 15. In some embodiments, the targeting sequence is SEQ ID NO: 16. In some embodiments, the targeting sequence is SEQ ID NO: 17. In some embodiments, the targeting sequence is SEQ ID NO: 18. In some embodiments, the targeting sequence is SEQ ID NO: 19. In some embodiments, the targeting sequence is SEQ ID NO: 20. In some embodiments, the targeting sequence is SEQ ID NO: 21. In some embodiments, the targeting sequence is SEQ ID NO: 22. In some embodiments, the targeting sequence is SEQ ID NO: 23. In some embodiments, the targeting sequence is SEQ ID NO: 24. In some embodiments, the targeting sequence is SEQ ID NO: 25. In some embodiments, the targeting sequence is SEQ ID NO: 26. In some embodiments, the targeting sequence is SEQ ID NO: 27. In some embodiments, the targeting sequence is SEQ ID NO: 28. In some embodiments, the targeting sequence is SEQ ID NO: 29. In some embodiments, the targeting sequence is SEQ ID NO: 30. In some embodiments, the targeting sequence is SEQ ID NO: 31. In some embodiments, the targeting sequence is SEQ ID NO: 32. In some embodiments, the targeting sequence is SEQ ID NO: 33. In some embodiments, the targeting sequence is SEQ ID NO: 34. In some embodiments, the targeting sequence is SEQ ID NO: 35. In some embodiments, the targeting sequence is SEQ ID NO: 36. In some embodiments, the targeting sequence is SEQ ID NO: 37. In some embodiments, the targeting sequence is SEQ ID NO: 38. In some embodiments, the targeting sequence is SEQ ID NO: 39. In some embodiments, the targeting sequence is SEQ ID NO: 40. In some embodiments, the targeting sequence is SEQ ID NO: 41. In some embodiments, the targeting sequence is SEQ ID NO: 42. In some embodiments, the targeting sequence is SEQ ID NO: 43. In some embodiments, the targeting sequence is SEQ ID NO: 44. In some embodiments, the targeting sequence is SEQ ID NO: 45. In some embodiments, the targeting sequence is SEQ ID NO: 46. In some embodiments, the targeting sequence is SEQ ID NO: 47. In some embodiments, the targeting sequence is SEQ ID NO: 48. In some embodiments, the targeting sequence is SEQ ID NO: 49. In some embodiments, the targeting sequence is SEQ ID NO: 50. In some embodiments, the targeting sequence is SEQ ID NO: 51. In some embodiments, the targeting sequence is SEQ ID NO: 52. In some embodiments, the targeting sequence is SEQ ID NO: 53. In some embodiments, the targeting sequence is SEQ ID NO: 54. In some embodiments, the targeting sequence is SEQ ID NO: 55. In some embodiments, the targeting sequence is SEQ ID NO: 56. In some embodiments, the targeting sequence is SEQ ID NO: 57. In some embodiments, the targeting sequence is SEQ ID NO: 58. In some embodiments, the targeting sequence is SEQ ID NO: 59. In some embodiments, the targeting sequence is SEQ ID NO: 60. In some embodiments, the targeting sequence is SEQ ID NO: 61. In some embodiments, the targeting sequence is SEQ ID NO: 62. In some embodiments, the targeting sequence is SEQ ID NO: 63. In some embodiments, the targeting sequence is SEQ ID NO: 64. In some embodiments, the targeting sequence is SEQ ID NO: 65. In some embodiments, the targeting sequence is SEQ ID NO: 66. In some embodiments, the targeting sequence is SEQ ID NO: 67. In some embodiments, the targeting sequence is SEQ ID NO: 68. In some embodiments, the targeting sequence is SEQ ID NO: 69. In some embodiments, the targeting sequence is SEQ ID NO: 70. In some embodiments, the targeting sequence is SEQ ID NO: 71. In some embodiments, the targeting sequence is SEQ ID NO: 72. In some embodiments, the targeting sequence is SEQ ID NO: 73. In some embodiments, the targeting sequence is SEQ ID NO: 74. In some embodiments, the targeting sequence is SEQ ID NO: 75. In some embodiments, the targeting sequence is SEQ ID NO: 76. In some embodiments, the targeting sequence is SEQ ID NO: 77. In some embodiments, the targeting sequence is SEQ ID NO: 78. In some embodiments, the targeting sequence is SEQ ID NO: 79. In some embodiments, the targeting sequence is SEQ ID NO: 80. In some embodiments, the targeting sequence is SEQ ID NO: 81. In some embodiments, the targeting sequence is SEQ ID NO: 82. In some embodiments, the targeting sequence is SEQ ID NO: 83. In some embodiments, the targeting sequence is SEQ ID NO: 84. In some embodiments, the targeting sequence is SEQ ID NO: 85. In some embodiments, the targeting sequence is SEQ ID NO: 86. In some embodiments, the targeting sequence is SEQ ID NO: 87. In some embodiments, the targeting sequence is SEQ ID NO: 88. In some embodiments, the targeting sequence is SEQ ID NO: 89. In some embodiments, the targeting sequence is SEQ ID NO: 90. In some embodiments, the targeting sequence is SEQ ID NO: 91. In some embodiments, the targeting sequence is SEQ ID NO: 92. In some embodiments, the targeting sequence is SEQ ID NO: 93. In some embodiments, the targeting sequence is SEQ ID NO: 94. In some embodiments, the targeting sequence is SEQ ID NO: 95. In some embodiments, the targeting sequence is SEQ ID NO: 96. In some embodiments, the targeting sequence is SEQ ID NO: 97. In some embodiments, the targeting sequence is SEQ ID NO: 98. In some embodiments, the targeting sequence is SEQ ID NO: 99. In some embodiments, the targeting sequence is SEQ ID NO: 100. In some embodiments, the targeting sequence is SEQ ID NO: 101. In some embodiments, the targeting sequence is SEQ ID NO: 102. In some embodiments, the targeting sequence is SEQ ID NO: 103. In some embodiments, the targeting sequence is SEQ ID NO: 104. In some embodiments, the targeting sequence is SEQ ID NO: 105. In some embodiments, the targeting sequence is SEQ ID NO: 106. In some embodiments, the targeting sequence is SEQ ID NO: 107. In some embodiments, the targeting sequence is SEQ ID NO: 108. In some embodiments, the targeting sequence is SEQ ID NO: 109. In some embodiments, the targeting sequence is SEQ ID NO: 110. In some embodiments, the targeting sequence is SEQ ID NO: 111. In some embodiments, the targeting sequence is SEQ ID NO: 112. In some embodiments, the targeting sequence is SEQ ID NO: 113. In some embodiments, the targeting sequence is SEQ ID NO: 114. In some embodiments, the targeting sequence is SEQ ID NO: 115. In some embodiments, the targeting sequence is SEQ ID NO: 116. In some embodiments, the targeting sequence is SEQ ID NO: 117. In some embodiments, the targeting sequence is SEQ ID NO: 118. In some embodiments, the targeting sequence is SEQ ID NO: 119. In some embodiments, the targeting sequence is SEQ ID NO: 120. In some embodiments, the targeting sequence is SEQ ID NO: 121. In some embodiments, the targeting sequence is SEQ ID NO: 122. In some embodiments, the targeting sequence is SEQ ID NO: 123. In some embodiments, the targeting sequence is SEQ ID NO: 124. In some embodiments, the targeting sequence is SEQ ID NO: 125.

In certain embodiments, the antisense oligomer, or a pharmaceutically acceptable salt thereof, has a targeting sequence comprising a sequence selected from:

SEQ ID NO: 11
(GTCTGGTGTCCTGTGAACAGAGATG);
SEQ ID NO: 12
(CTCTCTGTCTCTGATGTCTGGTGTC);
SEQ ID NO: 13
(AGACGGGTTGGCCCTTTGAATTTTT);
SEQ ID NO: 14
(TCTAGATAGCACCTGCCCAAAGGAA);
SEQ ID NO: 15
(ATCCTTTCTGCTCTTCCCGC);
SEQ ID NO: 16
(CATCCTTTCTGCTCTTCCCG);
SEQ ID NO: 17
(AGAGATGGCTGCCCCATCCTTTCTG);
SEQ ID NO: 20
(CAAGTCAGAGATGGCTGCCCCATCC);
SEQ ID NO: 23
(CATCCAAGTCAGAGATGGCT);
SEQ ID NO: 26
(ACCATCAGCATCCAAGTCAG);
SEQ ID NO: 27
(AGAGGCCACCACCACCATCAGCATC);
and
SEQ ID NO: 28
(CAGTGGCTGCAGTTGTGATGAACCA).

In certain embodiments, the antisense oligomer, or a pharmaceutically acceptable salt thereof, has a targeting sequence comprising a sequence selected from:

SEQ ID NO: 30
(GATATCTGAAACAGGTTAGG);
SEQ ID NO: 31
(AGATATCTGAAACAGGTTAG);
SEQ ID NO: 32
(GGGAGATATCTGAAACAGGT);
SEQ ID NO: 33
(AGGGAGATATCTGAAACAGG);
SEQ ID NO: 35
(TCAGCTGGCACTTGCCCAGCGACAC);
SEQ ID NO: 37
(AAGACCTTGTCGAAGCCCAGACTCT);
SEQ ID NO: 38
(CTTGTCGAAGCCCAGACT);
SEQ ID NO: 39
(ACCTTGTCGAAGCCCAGA);
SEQ ID NO: 40
(AGACCTTGTCGAAGCCCA);
SEQ ID NO: 44
(GGTTGTCTCTGTCATTGAAGCCCGA);
SEQ ID NO: 46
(GTCACTACAGACACCCAGTC);
SEQ ID NO: 47
(GGTCACTACAGACACCCAGT);
SEQ ID NO: 48
(ACCGTCAACACTGTCCCACA);
SEQ ID NO: 50
(GTACCGTCAACACTGTCCCA);
SEQ ID NO: 51
(ACGTACCGTCAACACTGTCC);
SEQ ID NO: 52
(GACGTACCGTCAACACTGTC);
and
SEQ ID NO: 53
(GGACGTACCGTCAACACTGT).

In certain embodiments, the antisense oligomer, or a pharmaceutically acceptable salt thereof, has a targeting sequence comprising a sequence selected from:

SEQ ID NO: 57
(TCATCTGCCAGGTAGAGGGTGTTGC);
SEQ ID NO: 58
(GGTCACGGATGATGATCTCA);
SEQ ID NO: 59
(AGGTCACGGATGATGATCTC);
SEQ ID NO: 61
(TTGATGTTGAGGTCACGGAT);
SEQ ID NO: 62
(TTTGATGTTGAGGTCACGGA);
SEQ ID NO: 64
(GGTAGGAGCATGCAAAGTTGATTTT);
SEQ ID NO: 66
(TTCAGGCTGACTTTCATGTCCAGGG);
SEQ ID NO: 67
(GCTGACTTTCATGTCCAGGG);
SEQ ID NO: 68
(GGTCTTCAGGCTGACTTTCA);
SEQ ID NO: 69
(CGGTCTTCAGGCTGACTTTC);
SEQ ID NO: 70
(GCGGTCTTCAGGCTGACTTT);
SEQ ID NO: 71
(GGCGGTCTTCAGGCTGACTT);
SEQ ID NO: 72
(CTGTAGGGCGGTCTTCAGGC);
SEQ ID NO: 73
(GCTGTAGGGCGGTCTTCAGG);
SEQ ID NO: 74
(GGCTGTAGGGCGGTCTTCAG);
SEQ ID NO: 75
(TGGCTGTAGGGCGGTCTTCA);
SEQ ID NO: 76
(TTGGCTGTAGGGCGGTCTTC);
SEQ ID NO: 77
(ATTGGCTGTAGGGCGGTCTT);
SEQ ID NO: 78
(CTGACCATTGGCTGTAGGGC);
SEQ ID NO: 79
(CCTGACCATTGGCTGTAGGGCGGTC);
SEQ ID NO: 80
(CACACCTGACCATTGGCTGT);
and
SEQ ID NO: 81
(CCACACCTGACCATTGGCTG).

In certain embodiments, the antisense oligomer, or a pharmaceutically acceptable salt thereof, has a targeting sequence comprising a sequence selected from:

SEQ ID NO: 82
(TTGAGTCTCTAGTGTGTGGGCATCT);
SEQ ID NO: 84
(TGGACGGAAAATCGGCCCTGGGAGG);
SEQ ID NO: 85
(CATCTGGACGGAAAATCGGC);
SEQ ID NO: 86
(ACATCTGGACGGAAAATCGG);
SEQ ID NO: 89
(AACCGGAACATCTGGACGGA);
SEQ ID NO: 90
(AAACCGGAACATCTGGACGG);
SEQ ID NO: 91
(CAAACCGGAACATCTGGACG);
SEQ ID NO: 92
(TTCCAGCAAACCGGAACATC);
SEQ ID NO: 93
(ATAGTTTCCAGCAAACCGGAACATC);
SEQ ID NO: 94
(TTTCCAGCAAACCGGAACAT);
SEQ ID NO: 95
(GTTTCCAGCAAACCGGAACA);
SEQ ID NO: 96
(AGTTTCCAGCAAACCGGAAC);
SEQ ID NO: 98
(ATAGTTTCCAGCAAACCGGA);
SEQ ID NO: 99
(CATAGTTTCCAGCAAACCGG);
SEQ ID NO: 100
(TCATAGTTTCCAGCAAACCG);
SEQ ID NO: 101
(AGGTCATAGTTTCCAGCAAA);
SEQ ID NO: 102
(GGTAGACTAGGTCATAGTTT);
SEQ ID NO: 103
(AGGTAGACTAGGTCATAGTT);
SEQ ID NO: 104
(CAGGTAGACTAGGTCATAGT);
SEQ ID NO: 105
(CTTCACAGTGCAGGTAGACTAGGTC);
SEQ ID NO: 106
(ACAGTGCAGGTAGACTAGGT);
SEQ ID NO: 107
(CACAGTGCAGGTAGACTAGG);
SEQ ID NO: 108
(TCACAGTGCAGGTAGACTAG);
SEQ ID NO: 109
(TTCACAGTGCAGGTAGACTA);
SEQ ID NO: 110
(GTCACAGAGATAGACTTCAC);
SEQ ID NO: 111
(TGTCACAGAGATAGACTTCA);
SEQ ID NO: 112
(GTGTCACAGAGATAGACTTC);
SEQ ID NO: 113
(TCATTCATGGTGTCACAGAGATAGA);
SEQ ID NO: 114
(TTCATGGTGTCACAGAGATA);
SEQ ID NO: 115
(ATTCATGGTGTCACAGAGAT);
and
SEQ ID NO: 120
(GAGTCAACTCACAGGCTTGCACTTT).

In certain embodiments, the antisense oligomer, or a pharmaceutically acceptable salt thereof, has a targeting sequence comprising a sequence selected from:

SEQ ID NO: 121
(AATCTGGTCCCAGAGCAGGTCTACA);
SEQ ID NO: 122
(TTCGGAATCTGGTCCCAGAGCAGGT);
SEQ ID NO: 123
(TGTGATGGGACCCAAGTTCAGGACA);
and
SEQ ID NO: 124
(AGCGAGTGGCTCTCTTACCTTTCCG).

In an embodiment, the target region is an intron/exon junction or an exon internal region of exon 2 (SEQ ID NO: 2). In certain embodiments, the target region of exon 2 is selected from H2A(−25−1), H2A(−18+2), H2A(−17+3), H2A(−16+4), H2A(−15+10), H2A(+1+25), H2A(+26+50), H2A(+51+75), H2A(+85+104), H2A(+86+105), H2A(+95+119), H2A(+96+115), H2A(+98+117), H2A(+101+125), H2A(+108+127), H2A(+109+128), H2A(+110+129), H2A(+113+132), H2A(+114+133), H2A(+118+137), H2A(+126+150), H2A(+151+175), and H2D (+15-10).

In some embodiments, the targeting sequence comprises a sequence selected from SEQ ID NOs: 7-29.

The target region of exon 2 is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175).

In another embodiment, the antisense oligomer, or a pharmaceutically acceptable salt thereof, has a targeting sequence complementary to a target region of exon 2 (SEQ ID NO: 2), wherein the targeting sequence comprises a sequence selected from:

SEQ ID NO: 11
(GTCTGGTGTCCTGTGAACAGAGATG);
SEQ ID NO: 12
(CTCTCTGTCTCTGATGTCTGGTGTC);
SEQ ID NO: 13
(AGACGGGTTGGCCCTTTGAATTTTT);
SEQ ID NO: 14
(TCTAGATAGCACCTGCCCAAAGGAA);
SEQ ID NO: 15
(ATCCTTTCTGCTCTTCCCGC);
SEQ ID NO: 16
(CATCCTTTCTGCTCTTCCCG);
SEQ ID NO: 17
(AGAGATGGCTGCCCCATCCTTTCTG);
SEQ ID NO: 20
(CAAGTCAGAGATGGCTGCCCCATCC);
SEQ ID NO: 23
(CATCCAAGTCAGAGATGGCT);
SEQ ID NO: 26
(ACCATCAGCATCCAAGTCAG);
SEQ ID NO: 27
(AGAGGCCACCACCACCATCAGCATC);
and
SEQ ID NO: 28
(CAGTGGCTGCAGTTGTGATGAACCA).

In some embodiments, the targeting sequence is SEQ ID NO: 11. In some embodiments, the targeting sequence is SEQ ID NO: 12. In some embodiments, the targeting sequence is SEQ ID NO: 13. In some embodiments, the targeting sequence is SEQ ID NO: 14. In some embodiments, the targeting sequence is SEQ ID NO: 15. In some embodiments, the targeting sequence is SEQ ID NO: 16. In some embodiments, the targeting sequence is SEQ ID NO: 17. In some embodiments, the targeting sequence is SEQ ID NO: 20. In some embodiments, the targeting sequence is SEQ ID NO: 23. In some embodiments, the targeting sequence is SEQ ID NO: 26. In some embodiments, the targeting sequence is SEQ ID NO: 27. In some embodiments, the targeting sequence is SEQ ID NO: 28.

In some embodiments, the target region is H2A(−15+10). In some embodiments, the target region is H2A(+1+25). In some embodiments, the target region is H2A(+26+50). In some embodiments, the target region is H2A(+51+75). In some embodiments, the target region is H2A(+85+104). In some embodiments, the target region is H2A(+86+105). In some embodiments, the target region is H2A(+95+119). In some embodiments, the target region is H2A(+101+125). In some embodiments, the target region is H2A(+110+129). In some embodiments, the target region is H2A(+118+137). In some embodiments, the target region is H2A(+126+150). In some embodiments, the target region is H2A(+151+175).

In an embodiment, the antisense oligomer, or a pharmaceutically acceptable salt thereof, has a targeting sequence complementary to a target region within the 51^{st} nucleotide to the 119^{th} nucleotide, as measured from the 5′ end of exon 2, of the human UMOD gene pre-mRNA. In certain embodiments, the antisense oligomer, or a pharmaceutically acceptable salt thereof, has a targeting sequence complementary to a target region comprised in SEQ ID NO: 154. In a further embodiment, the antisense oligomer, or a pharmaceutically acceptable salt thereof, has a targeting sequence complementary to a target region within the 51^{st} nucleotide to the 105^{th} nucleotide, as measured from the 5′ end of exon 2, of the human UMOD gene pre-mRNA. In certain embodiments, the antisense oligomer, or a pharmaceutically acceptable salt thereof, has a targeting sequence complementary to a target region comprised in SEQ ID NO: 155. In still a further embodiment, the target region is H2A(+51+75). In certain embodiments, the antisense oligomer, or a pharmaceutically acceptable salt thereof, has a targeting sequence comprising SEQ ID NO: 14.

In an embodiment, the antisense oligomer, or a pharmaceutically acceptable salt thereof, has a targeting sequence complementary to a target region within the 85^{th} nucleotide to the 119^{th} nucleotide, as measured from the 5′ end of exon 2, of the human UMOD gene pre-mRNA. In certain embodiments, the antisense oligomer, or a pharmaceutically acceptable salt thereof, has a targeting sequence complementary to a target region comprised in SEQ ID NO: 156. In a further embodiment, the target region is selected from H2A(+85+104), H2A(+86+105), and H2A(+95+119). In still a further embodiment, the antisense oligomer, or a pharmaceutically acceptable salt thereof, has a targeting sequence comprising a sequence selected from SEQ ID NOs: 15-17.

In an embodiment, the antisense oligomer, or a pharmaceutically acceptable salt thereof, has a targeting sequence complementary to a target region within the 15^{th} nucleotide of intron 1, as measured from the 5′ end of exon 2, and the 25^{th} nucleotide of exon 2, as measured from the 5′ end of exon 2, of the human UMOD gene pre-mRNA. In certain embodiments, the antisense oligomer, or a pharmaceutically acceptable salt thereof, has a targeting sequence complementary to a target region comprised in SEQ ID NO: 157. In a further embodiment, the target region is selected from H2A(−15+10) and H2A(+1+25). In certain embodiments, the antisense oligomer, or a pharmaceutically acceptable salt thereof, has a targeting sequence comprising SEQ ID NO: 11 or 12.

In an embodiment, the antisense oligomer, or a pharmaceutically acceptable salt thereof, has a targeting sequence complementary to a target region within the 118^{th} nucleotide to the 150^{th} nucleotide of exon 2, as measured from the 5′ end of exon 2, of the human UMOD gene pre-mRNA. In certain embodiments, the antisense oligomer, or a pharmaceutically acceptable salt thereof, has a targeting sequence complementary to a target region comprised in SEQ ID NO: 158. In a further embodiment, the target region is selected from H2A(+118+137) and H2A(+126+150). In certain embodiments, the antisense oligomer, or a pharmaceutically acceptable salt thereof, has a targeting sequence comprising SEQ ID NO: 26 or 27.

In an embodiment, the antisense oligomer, or a pharmaceutically acceptable salt thereof, has a targeting sequence complementary to a target region of exon 2 selected from H2A(+101+125), H2A(+110+129), and H2A(+151+175). In a further embodiment, the antisense oligomer, or a pharmaceutically acceptable salt thereof, has a targeting sequence selected from SEQ ID NOs: 20, 21, and 28.

In another embodiment, the target region is an intron/exon junction or an exon internal region of exon 5 (SEQ ID NO: 3). In certain embodiments, the target region of exon 5 is selected from H5A (−15+5), H5A (−14+6), H5A (−11+9), H5A (−10+10), H5A (−9+11), H5A(+50+67), H5A(+51+75), H5A(+52+69), H5A(+76+100), H5A(+78+95), H5A(+80+97), H5A(+82+99), H5A(+85+102), H5A(+86+103), H5A(+91+108), H5A(+126+150), H5A(+152+171), H5A(+153+172), H5A(+154+173), H5D (+18-2), H5D (+17-3), H5D (+16-4), H5D (+14-6), H5D (+13-7), H5D (+12-8), H5D (+11-9), and H5D (+10-10). In some embodiments, the antisense oligomer, or a pharmaceutically acceptable salt thereof, has a targeting sequence complementary to a target region of exon 5, wherein the targeting sequence comprises a sequence selected from SEQ ID NOs: 30-55. In some embodiments, the antisense oligomer, or a pharmaceutically acceptable salt thereof, has a targeting sequence complementary to a target region of exon 5, wherein the targeting sequence comprises a sequence selected from SEQ ID NOs: 30-33, 35, 37-40, 44, 46-48, and 50-53.

In certain embodiments, the target region of exon 5 is selected from H5A (−15+5), H5A (−14+6), H5A (−11+9), H5A (−10+10), H5A(+51+75), H5A(+76+100), H5A(+78+95), H5A(+80+97), H5A(+82+99), H5A(+126+150), H5A(+153+172), H5A(+154+173), H5D (+18-2), H5D (+16-4), H5D (+14-6), H5D (+13-7), and H5D (+12-8). In some embodiments, the antisense oligomer, or a pharmaceutically acceptable salt thereof, has a targeting sequence complementary to a target region of exon 5, wherein the targeting sequence comprises a sequence selected from:

SEQ ID NO: 30
(GATATCTGAAACAGGTTAGG);
SEQ ID NO: 31
(AGATATCTGAAACAGGTTAG);
SEQ ID NO: 32
(GGGAGATATCTGAAACAGGT);
SEQ ID NO: 33
(AGGGAGATATCTGAAACAGG);
SEQ ID NO: 35
(TCAGCTGGCACTTGCCCAGCGACAC);
SEQ ID NO: 37
(AAGACCTTGTCGAAGCCCAGACTCT);
SEQ ID NO: 38
(CTTGTCGAAGCCCAGACT);
SEQ ID NO: 39
(ACCTTGTCGAAGCCCAGA);
SEQ ID NO: 40
(AGACCTTGTCGAAGCCCA);
SEQ ID NO: 44
(GGTTGTCTCTGTCATTGAAGCCCGA);
SEQ ID NO: 46
(GTCACTACAGACACCCAGTC);
SEQ ID NO: 47
(GGTCACTACAGACACCCAGT);
SEQ ID NO: 48
(ACCGTCAACACTGTCCCACA);
SEQ ID NO: 50
(GTACCGTCAACACTGTCCCA);
SEQ ID NO: 51
(ACGTACCGTCAACACTGTCC);
SEQ ID NO: 52
(GACGTACCGTCAACACTGTC);
and
SEQ ID NO: 53
(GGACGTACCGTCAACACTGT).

In some embodiments, the targeting sequence is SEQ ID NO: 30. In some embodiments, the targeting sequence is SEQ ID NO: 31. In some embodiments, the targeting sequence is SEQ ID NO: 32. In some embodiments, the targeting sequence is SEQ ID NO: 33. In some embodiments, the targeting sequence is SEQ ID NO: 35. In some embodiments, the targeting sequence is SEQ ID NO: 37. In some embodiments, the targeting sequence is SEQ ID NO: 38. In some embodiments, the targeting sequence is SEQ ID NO: 39. In some embodiments, the targeting sequence is SEQ ID NO: 40. In some embodiments, the targeting sequence is SEQ ID NO: 44. In some embodiments, the targeting sequence is SEQ ID NO: 46. In some embodiments, the targeting sequence is SEQ ID NO: 47. In some embodiments, the targeting sequence is SEQ ID NO: 48. In some embodiments, the targeting sequence is SEQ ID NO: 50. In some embodiments, the targeting sequence is SEQ ID NO: 51. In some embodiments, the targeting sequence is SEQ ID NO: 52. In some embodiments, the targeting sequence is SEQ ID NO: 53.

In some embodiments, the target region is H5A (−15+5). In some embodiments, the target region is H5A (−14+6). In some embodiments, the target region is H5A (−11+9). In some embodiments, the target region is H5A (−10+10). In some embodiments, the target region is H5A(+51+75). In some embodiments, the target region is H5A(+76+100). In some embodiments, the target region is H5A(+78+95). In some embodiments, the target region is H5A(+80+97). In some embodiments, the target region is H5A(+82+99). In some embodiments, the target region is H5A(+126+150). In some embodiments, the target region is H5A(+153+172). In some embodiments, the target region is H5A(+154+173). In some embodiments, the target region is H5D (+18-2). In some embodiments, the target region is H5D (+16-4). In some embodiments, the target region is H5D (+14-6). In some embodiments, the target region is H5D (+13−7). In some embodiments, the target region is H5D (+12−8).

In an embodiment, the antisense oligomer, or a pharmaceutically acceptable salt thereof, has a targeting sequence complementary to a target region within the 15^{th} nucleotide of intron 4, as measured from the 5′ end of exon 5 and the 10^{th} nucleotide of exon 5, as measured from the 5′ end of exon 5, of the human UMOD gene pre-mRNA. In certain embodiments, the antisense oligomer, or a pharmaceutically acceptable salt thereof, has a targeting sequence complementary to a target region comprised in SEQ ID NO: 159. In a further embodiment, the target region is selected from H5A (−15+5), H5A (−14+6), H5A (−11+9), and H5A (−10+10). In still a further embodiment, the antisense oligomer, or a pharmaceutically acceptable salt thereof, has a targeting sequence comprising a sequence selected from SEQ ID NOs: 30-33.

In an embodiment, the antisense oligomer, or a pharmaceutically acceptable salt thereof, has a targeting sequence complementary to the target region H5A(+51+75). In a further embodiment, the antisense oligomer, or a pharmaceutically acceptable salt thereof, has a targeting sequence comprising SEQ ID NO: 35.

In an embodiment, the antisense oligomer, or a pharmaceutically acceptable salt thereof, has a targeting sequence complementary to a target region within the 76^{th} nucleotide to the 100^{th} nucleotide, as measured from the 5′ end of exon 5, of the human UMOD gene pre-mRNA. In certain embodiments, the antisense oligomer, or a pharmaceutically acceptable salt thereof, has a targeting sequence complementary to a target region comprised in SEQ ID NO: 37. In a further embodiment, the target region is selected from H5A(+76+100), H5A(+78+95), H5A(+80+97), and H5A(+82+99). In one embodiment, the target region is H5A(+76+100). In certain embodiments, the antisense oligomer, or a pharmaceutically acceptable salt thereof, has a targeting sequence comprising SEQ ID NO: 37.

In one embodiment, the target region is H5A(+78+95). In another embodiment, the target region is H5A(+78+95). In certain embodiments, the antisense oligomer, or a pharmaceutically acceptable salt thereof, has a targeting sequence comprising SEQ ID NO: 38.

In one embodiment, the target region is H5A(+80+97). In another embodiment, the target region is H5A(+80+97). In certain embodiments, the antisense oligomer, or a pharmaceutically acceptable salt thereof, has a targeting sequence comprising SEQ ID NO: 39.

In one embodiment, the target region is H5A(+82+99). In another embodiment, the target region is H5A(+82+99). In certain embodiments, the antisense oligomer, or a pharmaceutically acceptable salt thereof, has a targeting sequence comprising SEQ ID NO: 40.

In a further embodiment, the target region is H5A(+126+150). In certain embodiments, the antisense oligomer, or a pharmaceutically acceptable salt thereof, has a targeting sequence comprising SEQ ID NO: 44.

In an embodiment, the antisense oligomer, or a pharmaceutically acceptable salt thereof, has a targeting sequence complementary to a target region within the 153^{rd} nucleotide to the 173^{rd} nucleotide measured from the 5′ end of exon 5 (SEQ ID NO: 3) of the human UMOD gene pre-mRNA. In certain embodiments, the antisense oligomer, or a pharmaceutically acceptable salt thereof, has a targeting sequence complementary to a target region comprised in SEQ ID NO: 564. In a further embodiment, the target region is H5A(+153+172) and H5A(+154+173). In certain embodiments, the antisense oligomer, or a pharmaceutically acceptable salt thereof, has a targeting sequence comprising SEQ ID NO: 46 or 47.

In an embodiment, the antisense oligomer, or a pharmaceutically acceptable salt thereof, has a targeting sequence complementary to a target region within the 18^{th} nucleotide of exon 5 measured from 3′ end of exon 5 to the 8^{th} nucleotide of intron 5 measured from 3′ end of exon 5, of the human UMOD gene pre-mRNA. In certain embodiments, the antisense oligomer, or a pharmaceutically acceptable salt thereof, has a targeting sequence complementary to a target region comprised in SEQ ID NO: 160. In a further embodiment, the target region is selected from H5D (+18−2), H5D (+16-4), H5D (+14-6), H5D (+13-7), and H5D (+12-8). In still a further embodiment, the antisense oligomer, or a pharmaceutically acceptable salt thereof, has a targeting sequence comprising a sequence selected from SEQ ID NOs: 48 and 50-53.

In another embodiment, the target region is an intron/exon junction or an exon internal region of exon 6 (SEQ ID NO: 4). In certain embodiments, the target region of exon 6 is selected from H6A(+1+25), H6A(+26+50), H6A(+48+67), H6A(+49+68), H6A(+50+69), H6A(+58+77), H6A(+59+78), H6A(+60+79), H6A(+76+100), H6A(+79+98), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D (+24-1), H6D (+15-5), and H6D (+14-6). In some embodiments, the antisense oligomer, or a pharmaceutically acceptable salt thereof, has a targeting sequence complementary to a target region of exon 6, wherein the targeting sequence comprises a sequence selected from SEQ ID NOs: 56-81.

In certain embodiments, the target region of exon 6 is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D (+24-1), H6D (+15-5), and H6D (+14-6). In some embodiments, the antisense oligomer, or a pharmaceutically acceptable salt thereof, has a targeting sequence complementary to a target region of exon 6, wherein the targeting sequence comprises a

SEQ ID NO: 57
(TCATCTGCCAGGTAGAGGGTGTTGC);
SEQ ID NO: 58
(GGTCACGGATGATGATCTCA);
SEQ ID NO: 59
(AGGTCACGGATGATGATCTC);
SEQ ID NO: 61
(TTGATGTTGAGGTCACGGAT);
SEQ ID NO: 62
(TTTGATGTTGAGGTCACGGA);
SEQ ID NO: 64
(GGTAGGAGCATGCAAAGTTGATTTT);
SEQ ID NO: 66
(TTCAGGCTGACTTTCATGTCCAGGG);
SEQ ID NO: 67
(GCTGACTTTCATGTCCAGGG);
SEQ ID NO: 68
(GGTCTTCAGGCTGACTTTCA);
SEQ ID NO: 69
(CGGTCTTCAGGCTGACTTTC);
SEQ ID NO: 70
(GCGGTCTTCAGGCTGACTTT);
SEQ ID NO: 71
(GGCGGTCTTCAGGCTGACTT);
SEQ ID NO: 72
(CTGTAGGGCGGTCTTCAGGC);
SEQ ID NO: 73
(GCTGTAGGGCGGTCTTCAGG);
SEQ ID NO: 74
(GGCTGTAGGGCGGTCTTCAG);
SEQ ID NO: 75
(TGGCTGTAGGGCGGTCTTCA);
SEQ ID NO: 76
(TTGGCTGTAGGGCGGTCTTC);
SEQ ID NO: 77
(ATTGGCTGTAGGGCGGTCTT);
SEQ ID NO: 78
(CTGACCATTGGCTGTAGGGC);
SEQ ID NO: 79
(CCTGACCATTGGCTGTAGGGCGGTC);
SEQ ID NO: 80
(CACACCTGACCATTGGCTGT);
and
SEQ ID NO: 81
(CCACACCTGACCATTGGCTG).

In some embodiments, the targeting sequence is SEQ ID NO: 57. In some embodiments, the targeting sequence is SEQ ID NO: 58. In some embodiments, the targeting sequence is SEQ ID NO: 59. In some embodiments, the targeting sequence is SEQ ID NO: 61. In some embodiments, the targeting sequence is SEQ ID NO: 62. In some embodiments, the targeting sequence is SEQ ID NO: 64. In some embodiments, the targeting sequence is SEQ ID NO: 66. In some embodiments, the targeting sequence is SEQ ID NO: 67. In some embodiments, the targeting sequence is SEQ ID NO: 68. In some embodiments, the targeting sequence is SEQ ID NO: 69. In some embodiments, the targeting sequence is SEQ ID NO: 70. In some embodiments, the targeting sequence is SEQ ID NO: 71. In some embodiments, the targeting sequence is SEQ ID NO: 72. In some embodiments, the targeting sequence is SEQ ID NO: 73. In some embodiments, the targeting sequence is SEQ ID NO: 74. In some embodiments, the targeting sequence is SEQ ID NO: 75. In some embodiments, the targeting sequence is SEQ ID NO: 76. In some embodiments, the targeting sequence is SEQ ID NO: 77. In some embodiments, the targeting sequence is SEQ ID NO: 78. In some embodiments, the targeting sequence is SEQ ID NO: 79. In some embodiments, the targeting sequence is SEQ ID NO: 80. In some embodiments, the targeting sequence is SEQ ID NO: 81.

In some embodiments, the target region is H6A(+26+50). In some embodiments, the target region is H6A(+48+67). In some embodiments, the target region is H6A(+49+68). In some embodiments, the target region is H6A(+58+77). In some embodiments, the target region is H6A(+59+78). In some embodiments, the target region is H6A(+76+100). In some embodiments, the target region is H6A(+101+125). In some embodiments, the target region is H6A(+101+120). In some embodiments, the target region is H6A(+110+129). In some embodiments, the target region is H6A(+111+130). In some embodiments, the target region is H6A(+112+131). In some embodiments, the target region is H6A(+113+132). In some embodiments, the target region is H6A(+119+138). In some embodiments, the target region is H6A(+120+139). In some embodiments, the target region is H6A(+121+140). In some embodiments, the target region is H6A(+122+141). In some embodiments, the target region is H6A(+123+142). In some embodiments, the target region is H6A(+124+143). In some embodiments, the target region is H6A(+130+149). In some embodiments, the target region is H6D (+24-1). In some embodiments, the target region is H6D (+15-5). In some embodiments, the target region is H6D (+14-6).

In an embodiment, the antisense oligomer, or a pharmaceutically acceptable salt thereof, has a targeting sequence complementary to the target region H6A(+26+50). In a further embodiment, the antisense oligomer, or a pharmaceutically acceptable salt thereof, has a targeting sequence comprising SEQ ID NO: 57.

In an embodiment, the antisense oligomer, or a pharmaceutically acceptable salt thereof, has a targeting sequence complementary to a target region within the 48^{th} nucleotide to the 78^{th} nucleotide measured from the 5′ end of exon 6 of the human UMOD gene pre-mRNA. In certain embodiments, the antisense oligomer, or a pharmaceutically acceptable salt thereof, has a targeting sequence complementary to a target region comprised in SEQ ID NO: 161. In a further embodiment, the target region is selected from H6A(+48+67), H6A(+49+68), H6A(+58+77), and H6A(+59+78). In still a further embodiment, the antisense oligomer, or a pharmaceutically acceptable salt thereof, has a targeting sequence comprising a sequence selected from SEQ ID NOs: 58, 59, 61, and 62.

In another embodiment, the antisense oligomer, or a pharmaceutically acceptable salt thereof, has a targeting sequence complementary to a target region within the 58^{th} nucleotide to the 78^{th} nucleotide measured from the 5′ end of exon 6 of the human UMOD gene pre-mRNA. In certain embodiments, the antisense oligomer, or a pharmaceutically acceptable salt thereof, has a targeting sequence complementary to a target region comprised in SEQ ID NO: 162. In certain embodiments, the target region is H6A(+58+77) or H6A(+59+78). In one embodiment, the target region is H6A(+58+77). In certain embodiments, the antisense oligomer, or a pharmaceutically acceptable salt thereof, has a targeting sequence comprising SEQ ID NO: 61. In another embodiment, the target region is H6A(+59+78). In certain embodiments, the antisense oligomer, or a pharmaceutically acceptable salt thereof, has a targeting sequence comprising SEQ ID NO: 62.

In an embodiment, the antisense oligomer, or a pharmaceutically acceptable salt thereof, has a targeting sequence complementary to the target region H6A(+76+100). In a further embodiment, the antisense oligomer, or a pharmaceutically acceptable salt thereof, has a targeting sequence complementary to the target region H6A(+26+50). In certain embodiments, the targeting sequence comprises SEQ ID NO: 64. In certain embodiments, the targeting sequence comprises SEQ ID NO: 57.

In an embodiment, the antisense oligomer, or a pharmaceutically acceptable salt thereof, has a targeting sequence complementary to a target region within the 101^{st} nucleotide to the 132^{nd} nucleotide measured from the 5′ end of exon 6 of the human UMOD gene pre-mRNA. In certain embodiments, the antisense oligomer, or a pharmaceutically acceptable salt thereof, has a targeting sequence complementary to a target region comprised in SEQ ID NO: 163. In a further embodiment, the target region is H6A(+101+125), H6A(+101+120), H6A(+110+129), H6A(+111+130), H6A(+112+131), or H6A(+113+132). In still a further embodiment, the antisense oligomer, or a pharmaceutically acceptable salt thereof, has a targeting sequence comprising a sequence selected from SEQ ID NOs: 66-71.

In another embodiment, the antisense oligomer, or a pharmaceutically acceptable salt thereof, has a targeting sequence complementary to a target region within the 111^{th} nucleotide to the 132^{nd} nucleotide measured from the 5′ end of exon 6, of the human UMOD gene pre-mRNA. In certain embodiments, the antisense oligomer, or a pharmaceutically acceptable salt thereof, has a targeting sequence complementary to a target region comprised in SEQ ID NO: 164. In certain embodiments, the target region is H6A(+111+130), H6A(+112+131), or H6A(+113+132). In one embodiment, the target region is H6A(+111+130). In certain embodiments, the antisense oligomer, or a pharmaceutically acceptable salt thereof, has a targeting sequence comprising SEQ ID NO: 69. In another embodiment, the target region is H6A(+112+131). In certain embodiments, the antisense oligomer, or a pharmaceutically acceptable salt thereof, has a targeting sequence comprising SEQ ID NO: 70. In yet another embodiment, the target region is H6A(+113+132). In certain embodiments, the antisense oligomer, or a pharmaceutically acceptable salt thereof, has a targeting sequence comprising SEQ ID NO: 71.

In an embodiment, the antisense oligomer, or a pharmaceutically acceptable salt thereof, has a targeting sequence complementary to a target region within the 119^{th} nucleotide to the 149^{th} nucleotide measured from the 5′ end of exon 6 of the human UMOD gene pre-mRNA. In certain embodiments, the antisense oligomer, or a pharmaceutically acceptable salt thereof, has a targeting sequence complementary to a target region comprised in SEQ ID NO: 165. In another embodiment, the target region is H6A(+119+138), H6A(+120+139), H6A(+121+140), H6A(+122+141), H6A(+123+142), H6A(+124+143), or H6A(+130+149). In yet another embodiment, the antisense oligomer, or a pharmaceutically acceptable salt thereof, has a targeting sequence comprising a sequence selected from SEQ ID NOs: 72-78.

In a further embodiment, the antisense oligomer, or a pharmaceutically acceptable salt thereof, has a targeting sequence complementary to a target region within the 119^{th} nucleotide to the 141^{st} nucleotide measured from the 5′ end of exon 6, of the human UMOD gene pre-mRNA. In certain embodiments, the antisense oligomer, or a pharmaceutically acceptable salt thereof, has a targeting sequence complementary to a target region comprised in SEQ ID NO: 166. In an embodiment, the target region is H6A(+119+138), H6A(+120+139), H6A(+121+140), or H6A(+122+141). In another embodiment, the antisense oligomer, or a pharmaceutically acceptable salt thereof, has a targeting sequence comprising a sequence selected from SEQ ID NOs: 72-75.

In still a further embodiment, the antisense oligomer, or a pharmaceutically acceptable salt thereof, has a targeting sequence complementary to a target region within the 120^{th} nucleotide to the 141^{st} nucleotide measured from the 5′ end of exon 6 of the human UMOD gene pre-mRNA. In certain embodiments, the antisense oligomer, or a pharmaceutically acceptable salt thereof, has a targeting sequence complementary to a target region comprised in SEQ ID NO: 167. In certain embodiments, the target region is H6A(+120+139), H6A(+121+140), or H6A(+122+141). In one embodiment, the target region is H6A(+120+139). In yet another embodiment, the antisense oligomer, or a pharmaceutically acceptable salt thereof, has a targeting sequence comprising a sequence selected from SEQ ID NOs: 73-75. In certain embodiments, the antisense oligomer, or a pharmaceutically acceptable salt thereof, has a targeting sequence comprising SEQ ID NO: 73. In another embodiment, the target region is H6A(+121+140). In certain embodiments, the antisense oligomer, or a pharmaceutically acceptable salt thereof, has a targeting sequence comprising SEQ ID NO: 74. In yet another embodiment, the target region is H6A(+122+141). In certain embodiments, the antisense oligomer, or a pharmaceutically acceptable salt thereof, has a targeting sequence comprising SEQ ID NO: 75.

In still a further embodiment, the antisense oligomer, or a pharmaceutically acceptable salt thereof, has a targeting sequence complementary to a target region within the 123^{rd} nucleotide to the 149^{th} nucleotide measured from the 5′ end of exon 6, of the human UMOD gene pre-mRNA. In certain embodiments, the antisense oligomer, or a pharmaceutically acceptable salt thereof, has a targeting sequence complementary to a target region comprised in SEQ ID NO: 168. In an embodiment, the target region is H6A(+123+142), H6A(+124+143), or H6A(+130+149). In certain embodiments, the antisense oligomer, or a pharmaceutically acceptable salt thereof, has a targeting sequence comprising a sequence selected from SEQ ID NOs: 76-78.

In an embodiment, the antisense oligomer, or a pharmaceutically acceptable salt thereof, has a targeting sequence complementary to a target region within the 24^{th} nucleotide of exon 6 measured from 3′ end of exon 6 to the 6^{th} nucleotide of intron 6 measured from 3′ end of exon 6 of the human UMOD gene pre-mRNA. In certain embodiments, the antisense oligomer, or a pharmaceutically acceptable salt thereof, has a targeting sequence complementary to a target region comprised in SEQ ID NO: 169. In a further embodiment, the target region is selected from H6D (+24-1), H6D (+15-5), and H6D (+14-6). In still a further embodiment, the antisense oligomer, or a pharmaceutically acceptable salt thereof, has a targeting sequence comprising a sequence selected from SEQ ID NOs: 79-81.

In a further embodiment, the antisense oligomer, or a pharmaceutically acceptable salt thereof, has a targeting sequence complementary to a target region within the 15^{th} nucleotide of exon 6 measured from 3′ end of exon 6 to the 6^{th} nucleotide of intron 6 measured from 3′ end of exon 6, of the human UMOD gene pre-mRNA. In certain embodiments, the antisense oligomer, or a pharmaceutically acceptable salt thereof, has a targeting sequence complementary to a target region comprised in SEQ ID NO: 170. In some embodiments, the target region is selected from H6D (+15−5) and H6D (+14−6). In yet another embodiment, the antisense oligomer, or a pharmaceutically acceptable salt thereof, has a targeting sequence comprising a sequence selected from SEQ ID NOs: 80 and 81. In one embodiment, the target region is H6D (+15-5). In certain embodiments, the antisense oligomer, or a pharmaceutically acceptable salt thereof, has a targeting sequence comprising SEQ ID NO: 80. In another embodiment, the target region is H6D (+14-6). In certain embodiments, the antisense oligomer, or a pharmaceutically acceptable salt thereof, has a targeting sequence comprising SEQ ID NO: 81.

In some embodiments, the antisense oligomer, or a pharmaceutically acceptable salt thereof, has a targeting sequence complementary to a target region of exon 8. In yet another embodiment, the target region is an intron/exon junction or an exon internal region of exon 8 (SEQ ID NO: 5). In certain embodiments, the target region of exon 8 is selected from H8A (−2+23), H8A(+26+50), H8A(+51+75), H8A(+60+79), H8A(+61+80), H8A(+62+81), H8A(+63+82), H8A(+68+87), H8A(+69+88), H8A(+70+89), H8A(+76+95), H8A(+76+100), H8A(+77+96), H8A(+78+97), H8A(+79+98), H8A(+80+99), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), H8A(+133+152), H8A(+134+153), H8A(+139+158), H8D (+19-1), and H8D (+12-13). In some embodiments, the targeting sequence comprises a sequence selected from SEQ ID NOs: 82-120.

In certain embodiments, the target region of exon 8 is selected from H8A (−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D (+12-13). In some embodiments, the targeting sequence comprises a

SEQ ID NO: 82
(TTGAGTCTCTAGTGTGTGGGCATCT);
SEQ ID NO: 84
(TGGACGGAAAATCGGCCCTGGGAGG);
SEQ ID NO: 85
(CATCTGGACGGAAAATCGGC);
SEQ ID NO: 86
(ACATCTGGACGGAAAATCGG);
SEQ ID NO: 89
(AACCGGAACATCTGGACGGA);
SEQ ID NO: 90
(AAACCGGAACATCTGGACGG);
SEQ ID NO: 91
(CAAACCGGAACATCTGGACG);
SEQ ID NO: 92
(TTCCAGCAAACCGGAACATC);
SEQ ID NO: 93
(ATAGTTTCCAGCAAACCGGAACATC);
SEQ ID NO: 94
(TTTCCAGCAAACCGGAACAT);
SEQ ID NO: 95
(GTTTCCAGCAAACCGGAACA);
SEQ ID NO: 96
(AGTTTCCAGCAAACCGGAAC);
SEQ ID NO: 98
(ATAGTTTCCAGCAAACCGGA);
SEQ ID NO: 99
(CATAGTTTCCAGCAAACCGG);
SEQ ID NO: 100
(TCATAGTTTCCAGCAAACCG);
SEQ ID NO: 101
(AGGTCATAGTTTCCAGCAAA);
SEQ ID NO: 102
(GGTAGACTAGGTCATAGTTT);
SEQ ID NO: 103
(AGGTAGACTAGGTCATAGTT);
SEQ ID NO: 104
(CAGGTAGACTAGGTCATAGT);
SEQ ID NO: 105
(CTTCACAGTGCAGGTAGACTAGGTC);
SEQ ID NO: 106
(ACAGTGCAGGTAGACTAGGT);
SEQ ID NO: 107
(CACAGTGCAGGTAGACTAGG);
SEQ ID NO: 108
(TCACAGTGCAGGTAGACTAG);
SEQ ID NO: 109
(TTCACAGTGCAGGTAGACTA);
SEQ ID NO: 110
(GTCACAGAGATAGACTTCAC);
SEQ ID NO: 111
(TGTCACAGAGATAGACTTCA);
SEQ ID NO: 112
(GTGTCACAGAGATAGACTTC);
SEQ ID NO: 113
(TCATTCATGGTGTCACAGAGATAGA);
SEQ ID NO: 114
(TTCATGGTGTCACAGAGATA);
SEQ ID NO: 115
(ATTCATGGTGTCACAGAGAT);
and
SEQ ID NO: 120
(GAGTCAACTCACAGGCTTGCACTTT).

In some embodiments, the targeting sequence is SEQ ID NO: 82. In some embodiments, the targeting sequence is SEQ ID NO: 84. In some embodiments, the targeting sequence is SEQ ID NO: 85. In some embodiments, the targeting sequence is SEQ ID NO: 86. In some embodiments, the targeting sequence is SEQ ID NO: 89. In some embodiments, the targeting sequence is SEQ ID NO: 90. In some embodiments, the targeting sequence is SEQ ID NO: 91. In some embodiments, the targeting sequence is SEQ ID NO: 92. In some embodiments, the targeting sequence is SEQ ID NO: 93. In some embodiments, the targeting sequence is SEQ ID NO: 94. In some embodiments, the targeting sequence is SEQ ID NO: 95. In some embodiments, the targeting sequence is SEQ ID NO: 96. In some embodiments, the targeting sequence is SEQ ID NO: 98. In some embodiments, the targeting sequence is SEQ ID NO: 99. In some embodiments, the targeting sequence is SEQ ID NO: 100. In some embodiments, the targeting sequence is SEQ ID NO: 101. In some embodiments, the targeting sequence is SEQ ID NO: 102. In some embodiments, the targeting sequence is SEQ ID NO: 103. In some embodiments, the targeting sequence is SEQ ID NO: 104. In some embodiments, the targeting sequence is SEQ ID NO: 105. In some embodiments, the targeting sequence is SEQ ID NO: 106. In some embodiments, the targeting sequence is SEQ ID NO: 107. In some embodiments, the targeting sequence is SEQ ID NO: 108. In some embodiments, the targeting sequence is SEQ ID NO: 109. In some embodiments, the targeting sequence is SEQ ID NO: 110. In some embodiments, the targeting sequence is SEQ ID NO: 111. In some embodiments, the targeting sequence is SEQ ID NO: 112. In some embodiments, the targeting sequence is SEQ ID NO: 113. In some embodiments, the targeting sequence is SEQ ID NO: 114. In some embodiments, the targeting sequence is SEQ ID NO: 115. In some embodiments, the targeting sequence is SEQ ID NO: 120.

In some embodiments, the target region is selected from H8A (−2+23), H8A(+51+75), H8A(+60+79), H8A(+61+80), H8A(+68+87), H8A(+69+88), H8A(+70+89), H8A(+76+95, H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147, H8A(+129+148), and H8D (+12-13).

In some embodiments, the target region is H8A (−2+23). In some embodiments, the target region is H8A(+51+75). In some embodiments, the target region is H8A(+60+79). In some embodiments, the target region is H8A(+61+80). In some embodiments, the target region is H8A(+68+87). In some embodiments, the target region is H8A(+69+88). In some embodiments, the target region is H8A(+70+89). In some embodiments, the target region is H8A(+76+95). In some embodiments, the target region is H8A(+76+100). In some embodiments, the target region is H8A(+77+96). In some embodiments, the target region is H8A(+78+97). In some embodiments, the target region is H8A(+79+98). In some embodiments, the target region is H8A(+81+100). In some embodiments, the target region is H8A(+82+101). In some embodiments, the target region is H8A(+83+102). In some embodiments, the target region is H8A(+86+105). In some embodiments, the target region is H8A(+94+113). In some embodiments, the target region is H8A(+95+114). In some embodiments, the target region is H8A(+96+115). In some embodiments, the target region is H8A(+101+125). In some embodiments, the target region is H8A(+102+121). In some embodiments, the target region is H8A(+103+122). In some embodiments, the target region is H8A(+104+123). In some embodiments, the target region is H8A(+105+124). In some embodiments, the target region is H8A(+120+139). In some embodiments, the target region is H8A(+121+140). In some embodiments, the target region is H8A(+122+141). In some embodiments, the target region is H8A(+126+150). In some embodiments, the target region is H8A(+128+147). In some embodiments, the target region is H8A(+129+148). In some embodiments, the target region is H8D (+12-13).

In an embodiment, the antisense oligomer, or a pharmaceutically acceptable salt thereof, has a targeting sequence complementary to the target region H8A (−2+23). In a further embodiment, the antisense oligomer, or a pharmaceutically acceptable salt thereof, has a targeting sequence comprising SEQ ID NO: 82.

In some embodiments, the antisense oligomer, or a pharmaceutically acceptable salt thereof, has a targeting sequence complementary to a target region within the 51^{st} nucleotide to the 80^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 171. In a further embodiment, the target region is selected from H8A(+51+75), H8A(+60+79), and H8A(+61+80). In one embodiment, the antisense oligomer, or a pharmaceutically acceptable salt thereof, has a targeting sequence comprising a sequence selected from SEQ ID NOs: 84-86.

In an embodiment, the antisense oligomer, or a pharmaceutically acceptable salt thereof, has a targeting sequence complementary to a target region within the 68^{th} nucleotide to the 100^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 172. In another embodiment, the target region is H8A(+68+87), H8A(+69+88), H8A(+70+89), H8A(+76+95), H8A(+76+100), H8A(+77+96), H8A(+78+97), or H8A(+79+98). In yet another embodiment, the antisense oligomer, or a pharmaceutically acceptable salt thereof, has a targeting sequence comprising a sequence selected from SEQ ID NOs: 89-96.

In a further embodiment, the antisense oligomer, or a pharmaceutically acceptable salt thereof, has a targeting sequence complementary to a target region within the 76^{th} nucleotide to the 100^{th} nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 173. In an embodiment, the target region is H8A(+76+95), H8A(+77+96), H8A(+78+97), or H8A(+79+98). In another embodiment, the antisense oligomer, or a pharmaceutically acceptable salt thereof, has a targeting sequence comprising a sequence selected from SEQ ID NOs: 92 and 94-96.

In an embodiment, the antisense oligomer, or a pharmaceutically acceptable salt thereof, has a targeting sequence complementary to a target region within the 81^{st} nucleotide to the 105^{th} nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 174. In a further embodiment, the target region is selected from H8A(+81+100), H8A(+82+101), H8A(+83+102), and H8A(+86+105). In still a further embodiment, the antisense oligomer, or a pharmaceutically acceptable salt thereof, has a targeting sequence comprising a sequence selected from SEQ ID NOs: 98-101.

In an embodiment, the antisense oligomer, or a pharmaceutically acceptable salt thereof, has a targeting sequence complementary to a target region within the 94^{th} nucleotide to the 124^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 175. In a further embodiment, the target region is selected from H8A(+94+113), H8A(+95+114), H8A(+96+115), H8A(+101+125), H8A(+102+121), H8A(+103+122), H8A(+104+123), and H8A(+105+124). In still a further embodiment, the antisense oligomer, or a pharmaceutically acceptable salt thereof, has a targeting sequence comprising a sequence selected from SEQ ID NOs: 102-109.

In an embodiment, the antisense oligomer, or a pharmaceutically acceptable salt thereof, has a targeting sequence complementary to a target region within the 120^{th} nucleotide to the 148^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 176. In a further embodiment, the target region is selected from H8A(+120+139), H8A(+121+140), H8A(+122+141), H8A(+126+150), H8A(+128+147), and H8A(+129+148). In still a further embodiment, the antisense oligomer, or a pharmaceutically acceptable salt thereof, has a targeting sequence comprising a sequence selected from SEQ ID NOs: 110-115.

In a further embodiment, the antisense oligomer, or a pharmaceutically acceptable salt thereof, has a targeting sequence complementary to a target region within the 126^{th} nucleotide to the 148^{th} nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 177. In certain embodiments, the target region is selected from H8A(+126+150), H8A(+128+147), and H8A(+129+148). In some embodiments, the antisense oligomer, or a pharmaceutically acceptable salt thereof, has a targeting sequence comprising a sequence selected from SEQ ID NOs: 113-115. In one embodiment, the target region is H8A(+126+150). In some embodiments, the antisense oligomer, or a pharmaceutically acceptable salt thereof, has a targeting sequence comprising SEQ ID NO: 113. In one embodiment, the target region is H8A(+128+147). In some embodiments, the antisense oligomer, or a pharmaceutically acceptable salt thereof, has a targeting sequence comprising SEQ ID NO: 114. In one embodiment, the target region is H8A(+129+148). In some embodiments, the antisense oligomer, or a pharmaceutically acceptable salt thereof, has a targeting sequence comprising SEQ ID NO: 115.

In an embodiment, the antisense oligomer, or a pharmaceutically acceptable salt thereof, has a targeting sequence complementary to the target region H8D (+12-13). In another embodiment, the antisense oligomer, or a pharmaceutically acceptable salt thereof, has a targeting sequence comprising SEQ ID NO: 120.

In still another embodiment, the target region is an intron/exon junction or an exon internal region of exon 9 (SEQ ID NO: 6). In some embodiments, the target region of exon 9 is selected from H9A (−5+20), H9A(+1+25), H9A(+51+75), and H9D (+7-18). In some embodiments, the antisense oligomer, or a pharmaceutically acceptable salt thereof, has a targeting sequence complementary to a target region of exon 9. In another embodiment, the antisense oligomer, or a pharmaceutically acceptable salt thereof, has a targeting sequence comprising a sequence selected from SEQ ID NOs: 121-124. In certain embodiments, the targeting sequence comprises a sequence selected from:

SEQ ID NO: 121
(AATCTGGTCCCAGAGCAGGTCTACA);
SEQ ID NO: 122
(TTCGGAATCTGGTCCCAGAGCAGGT);
SEQ ID NO: 123
(TGTGATGGGACCCAAGTTCAGGACA);
and
SEQ ID NO: 124
(AGCGAGTGGCTCTCTTACCTTTCCG).

In some embodiments, the targeting sequence is SEQ ID NO: 121. In some embodiments, the targeting sequence is SEQ ID NO: 122. In some embodiments, the targeting sequence is SEQ ID NO: 123. In some embodiments, the targeting sequence is SEQ ID NO: 124.

In an embodiment, the antisense oligomer, or a pharmaceutically acceptable salt thereof, has a targeting sequence complementary to a target region within the 5^{th} nucleotide of intron 8 measured from the 5′ end of exon 9 and the 25^{th} nucleotide of exon 9 measured from the 5′ end of exon 9 of the human UMOD gene pre-mRNA. In certain embodiments, the antisense oligomer, or a pharmaceutically acceptable salt thereof, has a targeting sequence complementary to a target region comprised in SEQ ID NO: 178. In a further embodiment, the target region is H9A (−5+20) or H9A(+1+25). In some embodiments, the target region is H9A (−5+20). In some embodiments, the target region is H9A(+1+25). In some embodiments, the target region is H9A(+51+75). In some embodiments, the target region is H9D (+7-18).

In still a further embodiment, the antisense oligomer, or a pharmaceutically acceptable salt thereof, has a targeting sequence comprising a sequence selected from SEQ ID NOs: 121 and 122.

In an embodiment, the antisense oligomer, or a pharmaceutically acceptable salt thereof, has a target sequence complementary to the target region H9A(+51+75). In some embodiments, the antisense oligomer, or a pharmaceutically acceptable salt thereof, has a targeting sequence comprising SEQ ID NO: 123.

In another embodiment, the antisense oligomer, or a pharmaceutically acceptable salt thereof, has a target sequence complementary to the target region H9D (+7-18). In some embodiments, the antisense oligomer, or a pharmaceutically acceptable salt thereof, has a targeting sequence comprising SEQ ID NO: 124.

In some embodiments, the antisense oligomer, or a pharmaceutically acceptable salt thereof, has a targeting sequence comprising a sequence selected from SEQ ID NOs: 37, 32, 70, 74, 62, 81, 114, and 120. In some embodiments, the antisense oligomer, or a pharmaceutically acceptable salt thereof, has a targeting sequence comprising SEQ ID NO: 37. In some embodiments, the antisense oligomer, or a pharmaceutically acceptable salt thereof, has a targeting sequence comprising SEQ ID NO: 32. In some embodiments, the antisense oligomer, or a pharmaceutically acceptable salt thereof, has a targeting sequence comprising SEQ ID NO: 70. In some embodiments, the antisense oligomer, or a pharmaceutically acceptable salt thereof, has a targeting sequence comprising SEQ ID NO: 74. In some embodiments, the antisense oligomer, or a pharmaceutically acceptable salt thereof, has a targeting sequence comprising SEQ ID NO: 62. In some embodiments, the antisense oligomer, or a pharmaceutically acceptable salt thereof, has a targeting sequence comprising SEQ ID NO: 81. In some embodiments, the antisense oligomer, or a pharmaceutically acceptable salt thereof, has a targeting sequence comprising SEQ ID NO: 114. In some embodiments, the antisense oligomer, or a pharmaceutically acceptable salt thereof, has a targeting sequence comprising SEQ ID NO: 120.

In one embodiment, the targeting sequence is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent complementary to the target region. In another embodiment, the targeting sequence is at least 84%, at least 88%, or at least 92% complementary to the target region. In still another embodiment, the targeting sequence is at least 90% complementary to the target region. In yet another embodiment, the targeting sequence is at least 95% complementary to the target region. In still another embodiment, the targeting sequence is 100% complementary to the target region.

The antisense oligomer, or a pharmaceutically acceptable salt thereof, of the present disclosure can have a non-natural chemical backbone as described herein or known in the art. For example, the antisense oligomer, or a pharmaceutically acceptable salt thereof, may be a peptide nucleic acid (PNA), a locked nucleic acid, a phosphorodiamidate morpholino oligomer, a 2′-OMe phosphorothioate oligomer, or a combination thereof. In an embodiment, the antisense oligomer, or a pharmaceutically acceptable salt thereof, is a phosphorodiamidate morpholino oligomer. In another embodiment, the antisense oligomer, or a pharmaceutically acceptable salt thereof, is a peptide nucleic acid. In yet another embodiment, the antisense oligomer, or a pharmaceutically acceptable salt thereof, is a locked nucleic acid. In still another embodiment, the antisense oligomer, or a pharmaceutically acceptable salt thereof, is a 2′-OMe phosphorothioate oligomer. In still another embodiment, the antisense oligomer, or a pharmaceutically acceptable salt thereof, is a combination of any of the above oligomers, or pharmaceutically acceptable salts thereof, having a non-natural chemical backbone.

In some embodiments, the antisense oligomer, or a pharmaceutically acceptable salt thereof, is conjugated to (e.g., covalently attached to) or associated with (e.g., forms a complex with), a delivery agent such as, for example, a cell-penetrating peptide, an antibody, a fragment of an antibody, an antigen fragment of an antibody, at least one ligand, or a combination thereof.

In an embodiment, the antisense oligomer, or a pharmaceutically acceptable salt thereof, further comprises (e.g., is conjugated to) a cell-penetrating peptide (CPP). In certain embodiments, the antisense oligomer, or a pharmaceutically acceptable salt thereof, is covalently linked to a delivery agent selected from a cell-penetrating peptide, an antibody, a fragment of an antibody, an antigen binding agent, and a combination thereof. In an embodiment, the antisense oligomer, or a pharmaceutically acceptable salt thereof, further comprises a cell-penetrating peptide covalently linked to the antisense oligomer, or to a pharmaceutically acceptable salt thereof. In certain embodiments, the antisense oligomer, or a pharmaceutically acceptable salt thereof, is covalently linked to the cell-penetrating peptide via a linker selected from a direct bond, a glycine amino acid, a proline amino acid, glutamic acid amino acid, or an isoglutamine amino acid.

In another embodiment, the antisense oligomer, or a pharmaceutically acceptable salt thereof, is conjugated to a cell-penetrating peptide, wherein the cell-penetrating peptide is selected from rTAT (SEQ ID NO:179), TAT (SEQ ID NO: 180), R₉F₂ (SEQ ID NO: 181), R₅F₂R⁴ (SEQ ID NO: 182), R₄ (SEQ ID NO: 183), R₅ (SEQ ID NO: 184), R₆ (SEQ ID NO: 185), R₇ (SEQ ID NO: 136), R₈ (SEQ ID NO: 137), R₉ (SEQ ID NO: 138), (RXR)₄ (SEQ ID NO: 139), (RXR)₅ (SEQ ID NO: 140), (RXRRBR)₂ (SEQ ID NO: 141), (RAR)₄F₂ (SEQ ID NO: 142), (RGR)₄F₂ (SEQ ID NO: 143), and RBRBYLIQFRBRRBR (SEQ ID NO: 144), wherein A represents alanine, B represents beta-alanine (also represented as β-Ala or β), F represents phenylalanine, G represents glycine, I represents isoleucine, L represents leucine, Q represents glutamine, R represents arginine, X represents 6-aminohexanoic acid (also represented as Ahx or (x), and Y represents tyrosine. In yet another embodiment, the antisense oligomer, or a pharmaceutically acceptable salt thereof, is conjugated to an antibody, an antibody fragment, or an antigen fragment of an antibody.

The antisense oligomer, or a pharmaceutically acceptable salt thereof, of the present disclosure may be attached to (e.g., by covalent bond) or associated with (e.g., form a complex with) a delivery agent. In an embodiment, the antisense oligomer, or a pharmaceutically acceptable salt thereof, of the present disclosure is attached to the delivery agent via a linker. In an embodiment, the linker is a direct bond (e.g., covalent bond) from the antisense oligomer, or a pharmaceutically acceptable salt thereof, to a delivery agent. In another embodiment, the linker is an amino acid such as, for example, a glycine amino acid, a proline amino acid, or a glutamic amino acid. In a particular embodiment, the linker is a glycine amino acid or a proline amino acid. In certain embodiments, the delivery agent comprises an antibody, an antibody fragment, an antigen fragment of an antibody, at least one ligand, or a combination thereof. In yet another embodiment, the antisense oligomer, or a pharmaceutically acceptable salt thereof, is conjugated to an antibody, an antibody fragment, or an antigen fragment of an antibody.

### Formula (I)

In some embodiments, provided herein is an antisense oligomer of structural Formula (I):

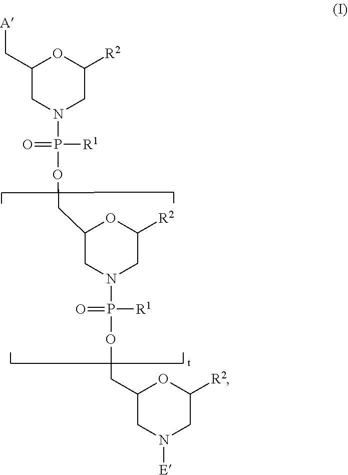

or a pharmaceutically acceptable salt thereof,
wherein:
A′ is selected from —OH,

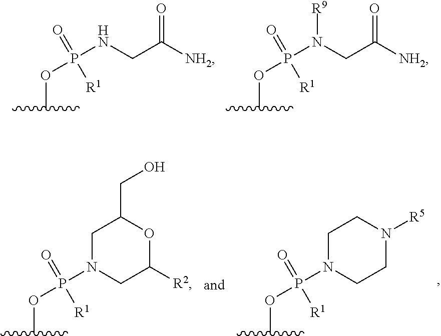

wherein:
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, —C₆₋₁₀-aryl-R⁶, 5-10 membered heteroaryl-Re, —C(O)O—(C₁₋₆-alkyl)-Re, —C(O)O-aryl-R⁶, —C(O)O-(5-10 membered heteroaryl)-R⁶, and

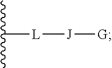

R⁶ is selected from —OH, —SH, and —NH₂, or R⁶ is O, S, or NH, each of which is covalently linked to a solid support;
R⁹ is C₁₋₆ alkyl;
each R¹ is independently selected from —OH and —N(R³)(R⁴), wherein each R³ and R⁴ is, independently at each occurrence, —H or —C₁₋₆-alkyl;
each R² is independently selected from a naturally or non-naturally occurring nucleobase, which, when taken together, forms a targeting sequence of 13 to 30 bases, that is complementary to a target region within a pre-mRNA of the human uromodulin (UMOD) gene (SEQ ID NO: 1) wherein the target region is an intron/exon junction or an exon internal region of the human UMOD gene pre-mRNA;
t is 11-28;
E′ is selected from —H, —C₁₋₆-alkyl, —C(O)C₁₋₆-alkyl, benzoyl, stearoyl, trityl, monomethoxytrityl, dimethoxytrityl, trimethoxytrityl,

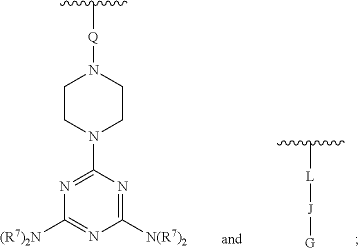

wherein:
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH) NH₂;
L is a linking amino acid, wherein L is covalently-linked by an amide bond to the C-terminus of J;
J is a cell-penetrating peptide; and
G is selected from —H, —C(O)C₁₋₆-alkyl, benzoyl, and stearoyl, wherein G is covalently-linked to J.

In some embodiments, t of Formula (I) is 11, 12, 13, 14, 15, 16, 17, 18, 19, 20, 21, 22, 23, 24, 25, 26, 27, or 28.

In an embodiment, t of Formula (I) is an integer from 11 to 28. In an embodiment, t of Formula (IA) is at least 11. In an embodiment, t of Formula (I) is up to 28.

In an embodiment, t is 11-28. In an embodiment, t is 11-27. In an embodiment, t is 11-26.

In an embodiment, t is 11-25. In an embodiment, t is 11-24. In an embodiment, t is 11-23. In an embodiment, t is 11-22. In an embodiment, t is 11-21. In an embodiment, t is 11-20. In an embodiment, t is 11-19. In an embodiment, t is 11-18. In an embodiment, t is 11-17. In an embodiment, t is 11-16. In an embodiment, t is 11-15. In an embodiment, t is 11-14. In an embodiment, t is 11-13. In an embodiment, t is 11-12. In an embodiment, t is 12-28. In an embodiment, t is 12-27. In an embodiment, t is 12-26. In an embodiment, t is 12-25. In an embodiment, t is 12-24. In an embodiment, t is 12-23. In an embodiment, t is 12-22. In an embodiment, t is 12-21. In an embodiment, t is 12-20. In an embodiment, t is 12-19. In an embodiment, t is 12-18. In an embodiment, t is 12-17. In an embodiment, t is 12-16. In an embodiment, t is 12-15. In an embodiment, t is 12-14. In an embodiment, t is 12-13. In an embodiment, t is 13-28. In an embodiment, t is 13-27. In an embodiment, t is 13-26. In an embodiment, t is 13-25. In an embodiment, t is 13-24. In an embodiment, t is 13-23. In an embodiment, t is 13-22. In an embodiment, t is 13-21. In an embodiment, t is 13-20. In an embodiment, t is 13-19. In an embodiment, t is 13-18. In an embodiment, t is 13-17. In an embodiment, t is 13-16. In an embodiment, t is 13-15. In an embodiment, t is 13-14. In an embodiment, t is 14-28. In an embodiment, t is 14-27. In an embodiment, t is 14-26. In an embodiment, t is 14-25. In an embodiment, t is 14-24. In an embodiment, t is 14-23. In an embodiment, t is 14-22. In an embodiment, t is 14-21. In an embodiment, t is 14-20. In an embodiment, t is 14-19. In an embodiment, t is 14-18. In an embodiment, t is 14-17. In an embodiment, t is 14-16. In an embodiment, t is 14-15. In an embodiment, t is 15-28. In an embodiment, t is 15-27. In an embodiment, t is 15-26. In an embodiment, t is 15-25. In an embodiment, t is 15-24. In an embodiment, t is 15-23. In an embodiment, t is 15-22. In an embodiment, t is 15-21. In an embodiment, t is 15-20. In an embodiment, t is 15-19. In an embodiment, t is 15-18. In an embodiment, t is 15-17. In an embodiment, t is 15-16. In an embodiment, t is 16-28. In an embodiment, t is 16-27. In an embodiment, t is 16-26. In an embodiment, t is 16-25. In an embodiment, t is 16-24. In an embodiment, t is 16-23. In an embodiment, t is 16-22. In an embodiment, t is 16-21. In an embodiment, t is 16-20. In an embodiment, t is 16-19. In an embodiment, t is 16-18. In an embodiment, t is 16-17. In an embodiment, t is 17-28. In an embodiment, t is 17-27. In an embodiment, t is 17-26. In an embodiment, t is 17-25. In an embodiment, t is 17-24. In an embodiment, t is 17-23. In an embodiment, t is 17-22. In an embodiment, t is 17-21. In an embodiment, t is 17-20. In an embodiment, t is 17-19. In an embodiment, t is 17-18. In an embodiment, t is 18-28. In an embodiment, t is 18-27. In an embodiment, t is 18-26. In an embodiment, t is 18-25. In an embodiment, t is 18-24. In an embodiment, t is 18-23. In an embodiment, t is 18-22. In an embodiment, t is 18-21. In an embodiment, t is 18-20. In an embodiment, t is 18-19. In an embodiment, t is 19-28. In an embodiment, t is 19-27. In an embodiment, t is 19-26. In an embodiment, t is 19-25. In an embodiment, t is 19-24. In an embodiment, t is 19-23. In an embodiment, t is 19-22. In an embodiment, t is 19-21. In an embodiment, t is 19-20. In an embodiment, t is 20-28. In an embodiment, t is 20-27. In an embodiment, t is 20-26. In an embodiment, t is 20-25. In an embodiment, t is 20-24. In an embodiment, t is 20-23. In an embodiment, t is 20-22. In an embodiment, t is 20-21. In an embodiment, t is 21-28. In an embodiment, t is 21-27. In an embodiment, t is 21-26. In an embodiment, t is 21-25. In an embodiment, t is 21-24. In an embodiment, t is 21-23. In an embodiment, t is 21-22. In an embodiment, t is 22-28. In an embodiment, t is 22-27. In an embodiment, t is 22-26. In an embodiment, t is 22-25. In an embodiment, t is 22-24. In an embodiment, t is 22-23. In an embodiment, t is 23-28. In an embodiment, t is 23-27. In an embodiment, t is 23-26. In an embodiment, t is 23-25. In an embodiment, t is 23-24. In an embodiment, t is 24-28. In an embodiment, t is 24-27. In an embodiment, t is 24-26. In an embodiment, t is 24-25. In an embodiment, t is 25-28. In an embodiment, t is 25-27. In an embodiment, t is 25-26. In another embodiment, t is 26-30. In an embodiment, t is 26-29. In an embodiment, t is 26-28. In an embodiment, t is 26-27. In an embodiment, t is 27-28.

In an embodiment, t is 11. In an embodiment, t is 12. In an embodiment, t is 13. In an embodiment, t is 14. In an embodiment, t is 15. In an embodiment, t is 16. In an embodiment, t is 17. In an embodiment, t is 18. In an embodiment, t is 19. In an embodiment, t is 20. In an embodiment, t is 21. In an embodiment, t is 22. In an embodiment, t is 23. In an embodiment, t is 24. In an embodiment, t is 25. In an embodiment, t is 26. In an embodiment, t is 27. In an embodiment, t is 28.

In some embodiments, each R² of Formula (I) is independently selected from a naturally or non-naturally occurring nucleobase, which, when taken together, forms a targeting sequence of from 13 to 30 bases in length, that is complementary to a target region within a pre-mRNA of the human uromodulin (UMOD) gene (SEQ ID NO: 1) wherein the target region is an intron/exon junction or an exon internal region of the human UMOD gene pre-mRNA. Each R² of Formula (I) taken together forms a targeting sequence of 13, 14, 15, 16, 17, 18, 19, 20, 21, 22, 23, 24, 25, 26, 27, 28, 29, or 30 bases. In some embodiments, each R² of Formula (I) taken together forms a targeting sequence of 18-27 bases. In some embodiments, each R² of Formula (I) taken together forms a targeting sequence of 20-25 bases.

In an embodiment, each R² is independently selected from a naturally or non-naturally occurring nucleobase, which, when taken together, forms a targeting sequence of from 13 to 30 bases in length. In an embodiment, each R² is independently selected from a naturally or non-naturally occurring nucleobase, which, when taken together, forms a targeting sequence at least 13 bases in length. In an embodiment, each R² is independently selected from a naturally or non-naturally occurring nucleobase, which, when taken together, forms a targeting sequence of up to 30 bases in length.

In an embodiment, each R² is independently selected from a naturally or non-naturally occurring nucleobase, which, when taken together, forms a targeting sequence of 13-29 bases in length. In an embodiment, each R² is independently selected from a naturally or non-naturally occurring nucleobase, which, when taken together, forms a targeting sequence of 13-28 bases in length. In an embodiment, each R² is independently selected from a naturally or non-naturally occurring nucleobase, which, when taken together, forms a targeting sequence of 13-27 bases in length. In an embodiment, each R² is independently selected from a naturally or non-naturally occurring nucleobase, which, when taken together, forms a targeting sequence of 13-26 bases in length. In an embodiment, each R² is independently selected from a naturally or non-naturally occurring nucleobase, which, when taken together, forms a targeting sequence of 13-25 bases in length. In an embodiment, each R² is independently selected from a naturally or non-naturally occurring nucleobase, which, when taken together, forms a targeting sequence of 13-24 bases in length. In an embodiment, each R² is independently selected from a naturally or non-naturally occurring nucleobase, which, when taken together, forms a targeting sequence of 13-23 bases in length. In an embodiment, each R² is independently selected from a naturally or non-naturally occurring nucleobase, which, when taken together, forms a targeting sequence of 13-22 bases in length. In an embodiment, each R² is independently selected from a naturally or non-naturally occurring nucleobase, which, when taken together, forms a targeting sequence of 13-21 bases in length. In an embodiment, each R² is independently selected from a naturally or non-naturally occurring nucleobase, which, when taken together, forms a targeting sequence or 13-20 bases in length. In an embodiment, each R² is independently selected from a naturally or non-naturally occurring nucleobase, which, when taken together, forms a targeting sequence or 13-19 bases in length. In an embodiment, each R² is independently selected from a naturally or non-naturally occurring nucleobase, which, when taken together, forms a targeting sequence of 13-18 bases in length. In an embodiment, each R² is independently selected from a naturally or non-naturally occurring nucleobase, which, when taken together, forms a targeting sequence of 13-17 bases in length. In an embodiment, each R² is independently selected from a naturally or non-naturally occurring nucleobase, which, when taken together, forms a targeting sequence of 13-16 bases in length. In an embodiment, each R² is independently selected from a naturally or non-naturally occurring nucleobase, which, when taken together, forms a targeting sequence of 13-15 bases in length. In an embodiment, each R² is independently selected from a naturally or non-naturally occurring nucleobase, which, when taken together, forms a targeting sequence of 13-14 bases in length.

In another embodiment, each R² is independently selected from a naturally or non-naturally occurring nucleobase, which, when taken together, forms a targeting sequence of 14-30 bases in length. In an embodiment, each R² is independently selected from a naturally or non-naturally occurring nucleobase, which, when taken together, forms a targeting sequence of 14-29 bases in length. In an embodiment, each R² is independently selected from a naturally or non-naturally occurring nucleobase, which, when taken together, forms a targeting sequence of 14-28 bases in length. In an embodiment, each R² is independently selected from a naturally or non-naturally occurring nucleobase, which, when taken together, forms a targeting sequence of 14-27 bases in length. In an embodiment, each R² is independently selected from a naturally or non-naturally occurring nucleobase, which, when taken together, forms a targeting sequence of 14-26 bases in length. In an embodiment, each R² is independently selected from a naturally or non-naturally occurring nucleobase, which, when taken together, forms a targeting sequence of 14-25 bases in length. In an embodiment, each R² is independently selected from a naturally or non-naturally occurring nucleobase, which, when taken together, forms a targeting sequence of 14-24 bases in length. In an embodiment, each R² is independently selected from a naturally or non-naturally occurring nucleobase, which, when taken together, forms a targeting sequence of 14-23 bases in length. In an embodiment, each R² is independently selected from a naturally or non-naturally occurring nucleobase, which, when taken together, forms a targeting sequence of 14-22 bases in length. In an embodiment, each R² is independently selected from a naturally or non-naturally occurring nucleobase, which, when taken together, forms a targeting sequence of 14-21 bases in length. In an embodiment, each R² is independently selected from a naturally or non-naturally occurring nucleobase, which, when taken together, forms a targeting sequence of 14-20 bases in length. In an embodiment, each R² is independently selected from a naturally or non-naturally occurring nucleobase, which, when taken together, forms a targeting sequence of 14-19 bases in length. In an embodiment, each R² is independently selected from a naturally or non-naturally occurring nucleobase, which, when taken together, forms a targeting sequence of 14-18 bases in length. In an embodiment, each R² is independently selected from a naturally or non-naturally occurring nucleobase, which, when taken together, forms a targeting sequence of 14-17 bases in length. In an embodiment, each R² is independently selected from a naturally or non-naturally occurring nucleobase, which, when taken together, forms a targeting sequence of 14-16 bases in length. In an embodiment, each R² is independently selected from a naturally or non-naturally occurring nucleobase, which, when taken together, forms a targeting sequence of 14-15 bases in length.

In another embodiment, each R² is independently selected from a naturally or non-naturally occurring nucleobase, which, when taken together, forms a targeting sequence of 15-30 bases in length. In an embodiment, each R² is independently selected from a naturally or non-naturally occurring nucleobase, which, when taken together, forms a targeting sequence of 15-29 bases in length. In an embodiment, each R² is independently selected from a naturally or non-naturally occurring nucleobase, which, when taken together, forms a targeting sequence of 15-28 bases in length. In an embodiment, each R² is independently selected from a naturally or non-naturally occurring nucleobase, which, when taken together, forms a targeting sequence of 15-27 bases in length. In an embodiment, each R² is independently selected from a naturally or non-naturally occurring nucleobase, which, when taken together, forms a targeting sequence of 15-26 bases in length. In an embodiment, each R² is independently selected from a naturally or non-naturally occurring nucleobase, which, when taken together, forms a targeting sequence of 15-25 bases in length. In an embodiment, each R² is independently selected from a naturally or non-naturally occurring nucleobase, which, when taken together, forms a targeting sequence of 15-24 bases in length. In an embodiment, each R² is independently selected from a naturally or non-naturally occurring nucleobase, which, when taken together, forms a targeting sequence of 15-23 bases in length. In an embodiment, each R² is independently selected from a naturally or non-naturally occurring nucleobase, which, when taken together, forms a targeting sequence of 15-22 bases in length. In an embodiment, each R² is independently selected from a naturally or non-naturally occurring nucleobase, which, when taken together, forms a targeting sequence of 15-21 bases in length. In an embodiment, each R² is independently selected from a naturally or non-naturally occurring nucleobase, which, when taken together, forms a targeting sequence of 15-20 bases in length. In an embodiment, each R² is independently selected from a naturally or non-naturally occurring nucleobase, which, when taken together, forms a targeting sequence of 15-19 bases in length. In an embodiment, each R² is independently selected from a naturally or non-naturally occurring nucleobase, which, when taken together, forms a targeting sequence of 15-18 bases in length. In an embodiment, each R² is independently selected from a naturally or non-naturally occurring nucleobase, which, when taken together, forms a targeting sequence of 15-17 bases in length. In an embodiment, each R² is independently selected from a naturally or non-naturally occurring nucleobase, which, when taken together, forms a targeting sequence of 15-16 bases in length.

In another embodiment, each R² is independently selected from a naturally or non-naturally occurring nucleobase, which, when taken together, forms a targeting sequence of 16-30 bases in length. In an embodiment, each R² is independently selected from a naturally or non-naturally occurring nucleobase, which, when taken together, forms a targeting sequence of 16-29 bases in length. In an embodiment, each R² is independently selected from a naturally or non-naturally occurring nucleobase, which, when taken together, forms a targeting sequence of 16-28 bases in length. In an embodiment, each R² is independently selected from a naturally or non-naturally occurring nucleobase, which, when taken together, forms a targeting sequence of 16-27 bases in length. In an embodiment, each R² is independently selected from a naturally or non-naturally occurring nucleobase, which, when taken together, forms a targeting sequence of 16-26 bases in length. In an embodiment, each R² is independently selected from a naturally or non-naturally occurring nucleobase, which, when taken together, forms a targeting sequence of 16-25 bases in length. In an embodiment, each R² is independently selected from a naturally or non-naturally occurring nucleobase, which, when taken together, forms a targeting sequence of 16-24 bases in length. In an embodiment, each R² is independently selected from a naturally or non-naturally occurring nucleobase, which, when taken together, forms a targeting sequence of 16-23 bases in length. In an embodiment, each R² is independently selected from a naturally or non-naturally occurring nucleobase, which, when taken together, forms a targeting sequence of 16-22 bases in length. In an embodiment, each R² is independently selected from a naturally or non-naturally occurring nucleobase, which, when taken together, forms a targeting sequence of 16-21 bases in length. In an embodiment, each R² is independently selected from a naturally or non-naturally occurring nucleobase, which, when taken together, forms a targeting sequence of 16-20 bases in length. In an embodiment, each R² is independently selected from a naturally or non-naturally occurring nucleobase, which, when taken together, forms a targeting sequence of 16-19 bases in length. In an embodiment, each R² is independently selected from a naturally or non-naturally occurring nucleobase, which, when taken together, forms a targeting sequence of 16-18 bases in length. In an embodiment, each R² is independently selected from a naturally or non-naturally occurring nucleobase, which, when taken together, forms a targeting sequence of 16-17 bases in length.

In another embodiment, each R² is independently selected from a naturally or non-naturally occurring nucleobase, which, when taken together, forms a targeting sequence of 17-30 bases in length. In an embodiment, each R² is independently selected from a naturally or non-naturally occurring nucleobase, which, when taken together, forms a targeting sequence of 17-29 bases in length. In an embodiment, each R² is independently selected from a naturally or non-naturally occurring nucleobase, which, when taken together, forms a targeting sequence of 17-28 bases in length. In an embodiment, each R² is independently selected from a naturally or non-naturally occurring nucleobase, which, when taken together, forms a targeting sequence of 17-27 bases in length. In an embodiment, each R² is independently selected from a naturally or non-naturally occurring nucleobase, which, when taken together, forms a targeting sequence of 17-26 bases in length. In an embodiment, each R² is independently selected from a naturally or non-naturally occurring nucleobase, which, when taken together, forms a targeting sequence of 17-25 bases in length. In an embodiment, each R² is independently selected from a naturally or non-naturally occurring nucleobase, which, when taken together, forms a targeting sequence of 17-24 bases in length. In an embodiment, each R² is independently selected from a naturally or non-naturally occurring nucleobase, which, when taken together, forms a targeting sequence of 17-23 bases in length. In an embodiment, each R² is independently selected from a naturally or non-naturally occurring nucleobase, which, when taken together, forms a targeting sequence of 17-22 bases in length. In an embodiment, each R² is independently selected from a naturally or non-naturally occurring nucleobase, which, when taken together, forms a targeting sequence of 17-21 bases in length. In an embodiment, each R² is independently selected from a naturally or non-naturally occurring nucleobase, which, when taken together, forms a targeting sequence of 17-20 bases in length. In an embodiment, each R² is independently selected from a naturally or non-naturally occurring nucleobase, which, when taken together, forms a targeting sequence of 17-19 bases in length. In an embodiment, each R² is independently selected from a naturally or non-naturally occurring nucleobase, which, when taken together, forms a targeting sequence of 17-18 bases in

In another embodiment, each R² is independently selected from a naturally or non-naturally occurring nucleobase, which, when taken together, forms a targeting sequence of 18-30 bases in length. In an embodiment, each R² is independently selected from a naturally or non-naturally occurring nucleobase, which, when taken together, forms a targeting sequence of 18-29 bases in length. In an embodiment, each R² is independently selected from a naturally or non-naturally occurring nucleobase, which, when taken together, forms a targeting sequence of 18-28 bases in length. In an embodiment, each R² is independently selected from a naturally or non-naturally occurring nucleobase, which, when taken together, forms a targeting sequence of 18-27 bases in length. In an embodiment, each R² is independently selected from a naturally or non-naturally occurring nucleobase, which, when taken together, forms a targeting sequence of 18-26 bases in length. In an embodiment, each R² is independently selected from a naturally or non-naturally occurring nucleobase, which, when taken together, forms a targeting sequence of 18-25 bases in length. In an embodiment, each R² is independently selected from a naturally or non-naturally occurring nucleobase, which, when taken together, forms a targeting sequence of 18-24 bases in length. In an embodiment, each R² is independently selected from a naturally or non-naturally occurring nucleobase, which, when taken together, forms a targeting sequence of 18-23 bases in length. In an embodiment, each R² is independently selected from a naturally or non-naturally occurring nucleobase, which, when taken together, forms a targeting sequence of 18-22 bases in length. In an embodiment, each R² is independently selected from a naturally or non-naturally occurring nucleobase, which, when taken together, forms a targeting sequence of 18-21 bases in length. In an embodiment, each R² is independently selected from a naturally or non-naturally occurring nucleobase, which, when taken together, forms a targeting sequence of 18-20 bases in length. In an embodiment, each R² is independently selected from a naturally or non-naturally occurring nucleobase, which, when taken together, forms a targeting sequence of 18-19 bases in length.

In another embodiment, each R² is independently selected from a naturally or non-naturally occurring nucleobase, which, when taken together, forms a targeting sequence of 19-30 bases in length. In an embodiment, each R² is independently selected from a naturally or non-naturally occurring nucleobase, which, when taken together, forms a targeting sequence of 19-29 bases in length. In an embodiment, each R² is independently selected from a naturally or non-naturally occurring nucleobase, which, when taken together, forms a targeting sequence of 19-28 bases in length. In an embodiment, each R² is independently selected from a naturally or non-naturally occurring nucleobase, which, when taken together, forms a targeting sequence of 19-27 bases in length. In an embodiment, each R² is independently selected from a naturally or non-naturally occurring nucleobase, which, when taken together, forms a targeting sequence of 19-26 bases in length. In an embodiment, each R² is independently selected from a naturally or non-naturally occurring nucleobase, which, when taken together, forms a targeting sequence of 19-25 bases in length. In an embodiment, each R² is independently selected from a naturally or non-naturally occurring nucleobase, which, when taken together, forms a targeting sequence of 19-24 bases in length. In an embodiment, each R² is independently selected from a naturally or non-naturally occurring nucleobase, which, when taken together, forms a targeting sequence of 19-23 bases in length. In an embodiment, each R² is independently selected from a naturally or non-naturally occurring nucleobase, which, when taken together, forms a targeting sequence of 19-22 bases in length. In an embodiment, each R² is independently selected from a naturally or non-naturally occurring nucleobase, which, when taken together, forms a targeting sequence of 19-21 bases in length. In an embodiment, each R² is independently selected from a naturally or non-naturally occurring nucleobase, which, when taken together, forms a targeting sequence of 19-20 bases in length.

In another embodiment, each R² is independently selected from a naturally or non-naturally occurring nucleobase, which, when taken together, forms a targeting sequence of 20-30 bases in length. In an embodiment, each R² is independently selected from a naturally or non-naturally occurring nucleobase, which, when taken together, forms a targeting sequence of 20-29 bases in length. In an embodiment, each R² is independently selected from a naturally or non-naturally occurring nucleobase, which, when taken together, forms a targeting sequence of 20-28 bases in length. In an embodiment, each R² is independently selected from a naturally or non-naturally occurring nucleobase, which, when taken together, forms a targeting sequence of 20-27 bases in length. In an embodiment, each R² is independently selected from a naturally or non-naturally occurring nucleobase, which, when taken together, forms a targeting sequence of 20-26 bases in length. In an embodiment, each R² is independently selected from a naturally or non-naturally occurring nucleobase, which, when taken together, forms a targeting sequence of 20-25 bases in length. In an embodiment, each R² is independently selected from a naturally or non-naturally occurring nucleobase, which, when taken together, forms a targeting sequence of 20-24 bases in length. In an embodiment, each R² is independently selected from a naturally or non-naturally occurring nucleobase, which, when taken together, forms a targeting sequence of 20-23 bases in length. In an embodiment, each R² is independently selected from a naturally or non-naturally occurring nucleobase, which, when taken together, forms a targeting sequence of 20-22 bases in length. In an embodiment, each R² is independently selected from a naturally or non-naturally occurring nucleobase, which, when taken together, forms a targeting sequence of 20-21 bases in length.

In another embodiment, each R² is independently selected from a naturally or non-naturally occurring nucleobase, which, when taken together, forms a targeting sequence of 21-30 bases in length. In an embodiment, each R² is independently selected from a naturally or non-naturally occurring nucleobase, which, when taken together, forms a targeting sequence of 21-29 bases in length. In an embodiment, each R² is independently selected from a naturally or non-naturally occurring nucleobase, which, when taken together, forms a targeting sequence of 21-28 bases in length. In an embodiment, each R² is independently selected from a naturally or non-naturally occurring nucleobase, which, when taken together, forms a targeting sequence of 21-27 bases in length. In an embodiment, each R² is independently selected from a naturally or non-naturally occurring nucleobase, which, when taken together, forms a targeting sequence of 21-26 bases in length. In an embodiment, each R² is independently selected from a naturally or non-naturally occurring nucleobase, which, when taken together, forms a targeting sequence of 21-25 bases in length. In an embodiment, each R² is independently selected from a naturally or non-naturally occurring nucleobase, which, when taken together, forms a targeting sequence of 21-24 bases in length. In an embodiment, each R² is independently selected from a naturally or non-naturally occurring nucleobase, which, when taken together, forms a targeting sequence of 21-23 bases in length. In an embodiment, each R² is independently selected from a naturally or non-naturally occurring nucleobase, which, when taken together, forms a targeting sequence of 21-22 bases in length.

In another embodiment, each R² is independently selected from a naturally or non-naturally occurring nucleobase, which, when taken together, forms a targeting sequence of 22-30 bases in length. In an embodiment, each R² is independently selected from a naturally or non-naturally occurring nucleobase, which, when taken together, forms a targeting sequence of 22-29 bases in length. In an embodiment, each R² is independently selected from a naturally or non-naturally occurring nucleobase, which, when taken together, forms a targeting sequence of 22-28 bases in length. In an embodiment, each R² is independently selected from a naturally or non-naturally occurring nucleobase, which, when taken together, forms a targeting sequence of 22-27 bases in length. In an embodiment, each R² is independently selected from a naturally or non-naturally occurring nucleobase, which, when taken together, forms a targeting sequence of 22-26 bases in length. In an embodiment, each R² is independently selected from a naturally or non-naturally occurring nucleobase, which, when taken together, forms a targeting sequence of 22-25 bases in length. In an embodiment, each R² is independently selected from a naturally or non-naturally occurring nucleobase, which, when taken together, forms a targeting sequence of 22-24 bases in length. In an embodiment, each R² is independently selected from a naturally or non-naturally occurring nucleobase, which, when taken together, forms a targeting sequence of 22-23 bases in length.

In another embodiment, each R² is independently selected from a naturally or non-naturally occurring nucleobase, which, when taken together, forms a targeting sequence of 23-30 bases in length. In an embodiment, each R² is independently selected from a naturally or non-naturally occurring nucleobase, which, when taken together, forms a targeting sequence of 23-29 bases in length. In an embodiment, each R² is independently selected from a naturally or non-naturally occurring nucleobase, which, when taken together, forms a targeting sequence of 23-28 bases in length. In an embodiment, each R² is independently selected from a naturally or non-naturally occurring nucleobase, which, when taken together, forms a targeting sequence of 23-27 bases in length. In an embodiment, each R² is independently selected from a naturally or non-naturally occurring nucleobase, which, when taken together, forms a targeting sequence of 23-26 bases in length. In an embodiment, each R² is independently selected from a naturally or non-naturally occurring nucleobase, which, when taken together, forms a targeting sequence of 23-25 bases in length. In an embodiment, each R² is independently selected from a naturally or non-naturally occurring nucleobase, which, when taken together, forms a targeting sequence of 23-24 bases in length.

In another embodiment, each R² is independently selected from a naturally or non-naturally occurring nucleobase, which, when taken together, forms a targeting sequence of 24-30 bases in length. In an embodiment, each R² is independently selected from a naturally or non-naturally occurring nucleobase, which, when taken together, forms a targeting sequence of 24-29 bases in length. In an embodiment, each R² is independently selected from a naturally or non-naturally occurring nucleobase, which, when taken together, forms a targeting sequence of 24-28 bases in length. In an embodiment, each R² is independently selected from a naturally or non-naturally occurring nucleobase, which, when taken together, forms a targeting sequence of 24-27 bases in length. In an embodiment, each R² is independently selected from a naturally or non-naturally occurring nucleobase, which, when taken together, forms a targeting sequence of 24-26 bases in length. In an embodiment, each R² is independently selected from a naturally or non-naturally occurring nucleobase, which, when taken together, forms a targeting sequence of 24-25 bases in length.

In another embodiment, each R² is independently selected from a naturally or non-naturally occurring nucleobase, which, when taken together, forms a targeting sequence of 25-30 bases in length. In an embodiment, each R² is independently selected from a naturally or non-naturally occurring nucleobase, which, when taken together, forms a targeting sequence of 25-29 bases in length. In an embodiment, each R² is independently selected from a naturally or non-naturally occurring nucleobase, which, when taken together, forms a targeting sequence of 25-28 bases in length. In an embodiment, each R² is independently selected from a naturally or non-naturally occurring nucleobase, which, when taken together, forms a targeting sequence of 25-27 bases in length. In an embodiment, each R² is independently selected from a naturally or non-naturally occurring nucleobase, which, when taken together, forms a targeting sequence of 25-26 bases in length.

In another embodiment, each R² is independently selected from a naturally or non-naturally occurring nucleobase, which, when taken together, forms a targeting sequence of 26-30 bases in length. In an embodiment, each R² is independently selected from a naturally or non-naturally occurring nucleobase, which, when taken together, forms a targeting sequence of 26-29 bases in length. In an embodiment, each R² is independently selected from a naturally or non-naturally occurring nucleobase, which, when taken together, forms a targeting sequence of 26-28 bases in length. In an embodiment, each R² is independently selected from a naturally or non-naturally occurring nucleobase, which, when taken together, forms a targeting sequence of 26-27 bases in length.

In another embodiment, each R² is independently selected from a naturally or non-naturally occurring nucleobase, which, when taken together, forms a targeting sequence of 27-30 bases in length. In an embodiment, each R² is independently selected from a naturally or non-naturally occurring nucleobase, which, when taken together, forms a targeting sequence of 27-29 bases in length. In an embodiment, each R² is independently selected from a naturally or non-naturally occurring nucleobase, which, when taken together, forms a targeting sequence of 27-28 bases in length.

In another embodiment, each R² is independently selected from a naturally or non-naturally occurring nucleobase, which, when taken together, forms a targeting sequence of 28-30 bases in length. In an embodiment, each R² is independently selected from a naturally or non-naturally occurring nucleobase, which, when taken together, forms a targeting sequence of 28-29 bases in length. In another embodiment, each R² is independently selected from a naturally or non-naturally occurring nucleobase, which, when taken together, forms a targeting sequence of 28-30 bases in length. In an embodiment, each R² is independently selected from a naturally or non-naturally occurring nucleobase, which, when taken together, forms a targeting sequence of 29-30 bases in length.

In another embodiment, each R² is independently selected from a naturally or non-naturally occurring nucleobase, which, when taken together, forms a targeting sequence of 13 bases in length.

In another embodiment, each R² is independently selected from a naturally or non-naturally occurring nucleobase, which, when taken together, forms a targeting sequence of 14 bases in length. In another embodiment, each R² is independently selected from a naturally or non-naturally occurring nucleobase, which, when taken together, forms a targeting sequence of 15 bases in length. In another embodiment, each R² is independently selected from a naturally or non-naturally occurring nucleobase, which, when taken together, forms a targeting sequence of 16 bases in length. In another embodiment, each R² is independently selected from a naturally or non-naturally occurring nucleobase, which, when taken together, forms a targeting sequence of 17 bases in length. In another embodiment, each R² is independently selected from a naturally or non-naturally occurring nucleobase, which, when taken together, forms a targeting sequence of 18 bases in length. In another embodiment, each R² is independently selected from a naturally or non-naturally occurring nucleobase, which, when taken together, forms a targeting sequence of 19 bases in length. In another embodiment, each R² is independently selected from a naturally or non-naturally occurring nucleobase, which, when taken together, forms a targeting sequence of 20 bases in length. In another embodiment, each R² is independently selected from a naturally or non-naturally occurring nucleobase, which, when taken together, forms a targeting sequence of 21 bases in length. In another embodiment, each R² is independently selected from a naturally or non-naturally occurring nucleobase, which, when taken together, forms a targeting sequence of 22 bases in length. In another embodiment, each R² is independently selected from a naturally or non-naturally occurring nucleobase, which, when taken together, forms a targeting sequence of 23 bases in length. In another embodiment, each R² is independently selected from a naturally or non-naturally occurring nucleobase, which, when taken together, forms a targeting sequence of 24 bases in length. In another embodiment, each R² is independently selected from a naturally or non-naturally occurring nucleobase, which, when taken together, forms a targeting sequence of 25 bases in length. In another embodiment, each R² is independently selected from a naturally or non-naturally occurring nucleobase, which, when taken together, forms a targeting sequence of 26 bases in length. In another embodiment, each R² is independently selected from a naturally or non-naturally occurring nucleobase, which, when taken together, forms a targeting sequence of 27 bases in length. In another embodiment, each R² is independently selected from a naturally or non-naturally occurring nucleobase, which, when taken together, forms a targeting sequence of 28 bases in length. In another embodiment, each R² is independently selected from a naturally or non-naturally occurring nucleobase, which, when taken together, forms a targeting sequence of 29 bases in length. In another embodiment, each R² is independently selected from a naturally or non-naturally occurring nucleobase, which, when taken together, forms a targeting sequence of 30 bases in length.

In an embodiment, E′ of Formula (I) is selected from H, —C₁₋₆-alkyl, —C(O)C₁₋₆-alkyl, benzoyl, stearoyl, trityl, monomethoxytrityl, dimethoxytrityl, trimethoxytrityl, and

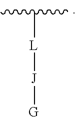

In a further embodiment, E′ of Formula (I) is selected from —H, —C(O) CH₃, benzoyl, stearoyl, trityl, 4-methoxytrityl, and

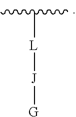

In an embodiment, A′ of Formula (I) is selected from:

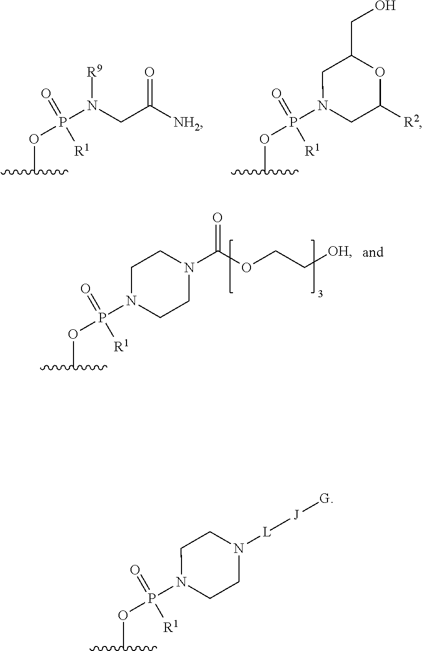

In an embodiment, at least one of the following of Formula (I) is true:
A′ is

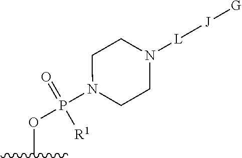

or (2) E′ is

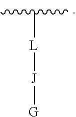

In an embodiment of Formula (I), A′ is selected from:

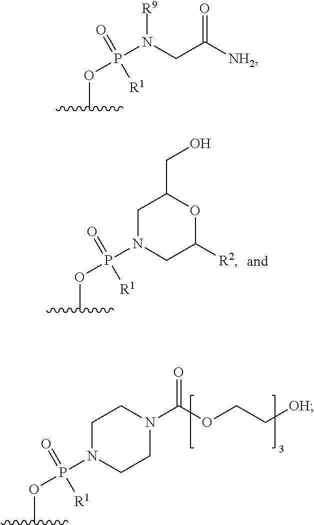

and E′ is

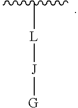

In an embodiment of Formula (I), A′ is

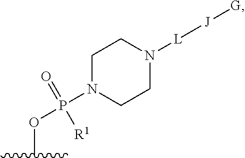

and
E′ is selected from H, —C(O) CH₃, trityl, 4-methoxytrityl, benzoyl, and stearoyl.

In an embodiment, each R¹ of Formula (I) is-N(CH₃)₂. In an embodiment, L of Formula (I) is glycine, proline, or β-alanine. In an embodiment, L of Formula (I) is glycine. In an embodiment, L of Formula (I) is proline. In an embodiment, L of Formula (I) is β-alanine.

In an embodiment, J of Formula (I) is selected from rTAT (SEQ ID NO: 179), TAT (SEQ ID NO: 180), R₉F₂ (SEQ ID NO: 181), R₅F₂R₄ (SEQ ID NO: 182), R₄ (SEQ ID NO: 183), R₅ (SEQ ID NO: 184), R₆ (SEQ ID NO: 185), R₇ (SEQ ID NO: 136), R₈ (SEQ ID NO: 137), R₉ (SEQ ID NO: 138), (RXR)₄ (SEQ ID NO: 139), (RXR)₅ (SEQ ID NO: 140), (RXRRBR)₂ (SEQ ID NO: 141), (RAR)₄F₂ (SEQ ID NO: 142), (RGR)₄F₂ (SEQ ID NO: 143), and RBRBYLIQFRBRRBR (SEQ ID NO: 144), wherein A represents alanine, B represents β-alanine (also represented as β-Ala or β), F represents phenylalanine, G represents glycine, R represents arginine, and X represents 6-aminohexanoic acid (also represented as Ahx or α).

In an embodiment, G of Formula (I) is selected from H, —C(O) CH₃, benzoyl, and stearoyl. In an embodiment, G of Formula (I) is H or —C(O) CH₃. In an embodiment, G of Formula (I) is H. In an embodiment, G of Formula (I) is —C(O) CH₃. In certain embodiments of the antisense oligomer, or pharmaceutically acceptable salt thereof, of structural Formula (I), the salt is an HCl salt.

### Formula (IA)

In some embodiments, provided herein is an antisense oligomer of Formula (I) that is structural Formula (IA):

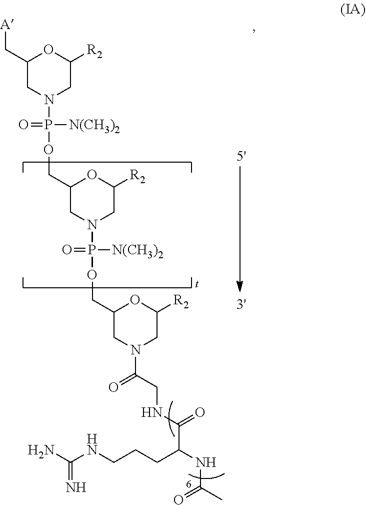

or a pharmaceutically acceptable salt thereof,
wherein:
A′ is a moiety selected from:

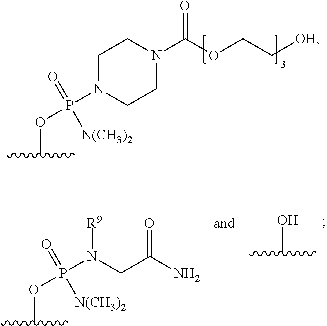

each R² is independently selected from a naturally or non-naturally occurring nucleobase, which, when taken together, forms a targeting sequence of 13 to 30 bases, that is complementary to a target region within a pre-mRNA of the human uromodulin (UMOD) gene (SEQ ID NO: 1) wherein the target region is an intron/exon junction or an exon internal region of the human UMOD gene pre-mRNA;
R⁹ is C₁₋₆ alkyl; and
t is 11-28.

In some embodiments, t of Formula (IA) is 11, 12, 13, 14, 15, 16, 17, 18, 19, 20, 21, 22, 23, 24, 25, 26, 27, or 28.

In an embodiment, t of Formula (IA) is an integer from 11 to 28. In an embodiment, t of Formula (IA) Is at least 11. In an embodiment, t of Formula (IA) is up to 28.

In an embodiment, t is 11-28. In an embodiment, t is 11-27. In an embodiment, t is 11-26.

In an embodiment, t is 11-25. In an embodiment, t is 11-24. In an embodiment, t is 11-23. In an embodiment, t is 11-22. In an embodiment, t is 11-21. In an embodiment, t is 11-20. In an embodiment, t is 11-19. In an embodiment, t is 11-18. In an embodiment, t is 11-17. In an embodiment, t is 11-16. In an embodiment, t is 11-15. In an embodiment, t is 11-14. In an embodiment, t is 11-13. In an embodiment, t is 11-12. In an embodiment, t is 12-28. In an embodiment, t is 12-27. In an embodiment, t is 12-26. In an embodiment, t is 12-25. In an embodiment, t is 12-24. In an embodiment, t is 12-23. In an embodiment, t is 12-22. In an embodiment, t is 12-21. In an embodiment, t is 12-20. In an embodiment, t is 12-19. In an embodiment, t is 12-18. In an embodiment, t is 12-17. In an embodiment, t is 12-16. In an embodiment, t is 12-15. In an embodiment, t is 12-14. In an embodiment, t is 12-13. In an embodiment, t is 13-28. In an embodiment, t is 13-27. In an embodiment, t is 13-26. In an embodiment, t is 13-25. In an embodiment, t is 13-24. In an embodiment, t is 13-23. In an embodiment, t is 13-22. In an embodiment, t is 13-21. In an embodiment, t is 13-20. In an embodiment, t is 13-19. In an embodiment, t is 13-18. In an embodiment, t is 13-17. In an embodiment, t is 13-16. In an embodiment, t is 13-15. In an embodiment, t is 13-14. In an embodiment, t is 14-28. In an embodiment, t is 14-27. In an embodiment, t is 14-26. In an embodiment, t is 14-25. In an embodiment, t is 14-24. In an embodiment, t is 14-23. In an embodiment, t is 14-22. In an embodiment, t is 14-21. In an embodiment, t is 14-20. In an embodiment, t is 14-19. In an embodiment, t is 14-18. In an embodiment, t is 14-17. In an embodiment, t is 14-16. In an embodiment, t is 14-15. In an embodiment, t is 15-28. In an embodiment, t is 15-27. In an embodiment, t is 15-26. In an embodiment, t is 15-25. In an embodiment, t is 15-24. In an embodiment, t is 15-23. In an embodiment, t is 15-22. In an embodiment, t is 15-21. In an embodiment, t is 15-20. In an embodiment, t is 15-19. In an embodiment, t is 15-18. In an embodiment, t is 15-17. In an embodiment, t is 15-16. In an embodiment, t is 16-28. In an embodiment, t is 16-27. In an embodiment, t is 16-26. In an embodiment, t is 16-25. In an embodiment, t is 16-24. In an embodiment, t is 16-23. In an embodiment, t is 16-22. In an embodiment, t is 16-21. In an embodiment, t is 16-20. In an embodiment, t is 16-19. In an embodiment, t is 16-18. In an embodiment, t is 16-17. In an embodiment, t is 17-28. In an embodiment, t is 17-27. In an embodiment, t is 17-26. In an embodiment, t is 17-25. In an embodiment, t is 17-24. In an embodiment, t is 17-23. In an embodiment, t is 17-22. In an embodiment, t is 17-21. In an embodiment, t is 17-20. In an embodiment, t is 17-19. In an embodiment, t is 17-18. In an embodiment, t is 18-28. In an embodiment, t is 18-27. In an embodiment, t is 18-26. In an embodiment, t is 18-25. In an embodiment, t is 18-24. In an embodiment, t is 18-23. In an embodiment, t is 18-22. In an embodiment, t is 18-21. In an embodiment, t is 18-20. In an embodiment, t is 18-19. In an embodiment, t is 19-28. In an embodiment, t is 19-27. In an embodiment, t is 19-26. In an embodiment, t is 19-25. In an embodiment, t is 19-24. In an embodiment, t is 19-23. In an embodiment, t is 19-22. In an embodiment, t is 19-21. In an embodiment, t is 19-20. In an embodiment, t is 20-28. In an embodiment, t is 20-27. In an embodiment, t is 20-26. In an embodiment, t is 20-25. In an embodiment, t is 20-24. In an embodiment, t is 20-23. In an embodiment, t is 20-22. In an embodiment, t is 20-21. In an embodiment, t is 21-28. In an embodiment, t is 21-27. In an embodiment, t is 21-26. In an embodiment, t is 21-25. In an embodiment, t is 21-24. In an embodiment, t is 21-23. In an embodiment, t is 21-22. In an embodiment, t is 22-28. In an embodiment, t is 22-27. In an embodiment, t is 22-26. In an embodiment, t is 22-25. In an embodiment, t is 22-24. In an embodiment, t is 22-23. In an embodiment, t is 23-28. In an embodiment, t is 23-27. In an embodiment, t is 23-26. In an embodiment, t is 23-25. In an embodiment, t is 23-24. In an embodiment, t is 24-28. In an embodiment, t is 24-27. In an embodiment, t is 24-26. In an embodiment, t is 24-25. In an embodiment, t is 25-28. In an embodiment, t is 25-27. In an embodiment, t is 25-26. In another embodiment, t is 26-30. In an embodiment, t is 26-29. In an embodiment, t is 26-28. In an embodiment, t is 26-27. In an embodiment, t is 27-28.

In an embodiment, t is 11. In an embodiment, t is 12. In an embodiment, t is 13. In an embodiment, t is 14. In an embodiment, tis 15. In an embodiment, t is 16. In an embodiment, t is 17. In an embodiment, t is 18. In an embodiment, t is 19. In an embodiment, t is 20. In an embodiment, t is 21. In an embodiment, t is 22. In an embodiment, t is 23. In an embodiment, t is 24. In an embodiment, t is 25. In an embodiment, t is 26. In an embodiment, t is 27. In an embodiment, t is 28.

In some embodiments, each R² of Formula (IA) taken together forms a targeting sequence of 13, 14, 15, 16, 17, 18, 19, 20, 21, 22, 23, 24, 25, 26, 27, 28, 29, or 30 bases. In some embodiments, each R² of Formula (IA) taken together forms a targeting sequence of 18-27 bases. In some embodiments, each R² of Formula (IA) taken together forms a targeting sequence of 20-25 bases.

In an embodiment, each R² is independently selected from a naturally or non-naturally occurring nucleobase, which, when taken together, forms a targeting sequence of from 13 to 30 bases in length. In an embodiment, each R² is independently selected from a naturally or non-naturally occurring nucleobase, which, when taken together, forms a targeting sequence at least 13 bases in length. In an embodiment, each R² is independently selected from a naturally or non-naturally occurring nucleobase, which, when taken together, forms a targeting sequence of up to 30 bases in length.

In an embodiment, each R² is independently selected from a naturally or non-naturally occurring nucleobase, which, when taken together, forms a targeting sequence of 13-29 bases in length. In an embodiment, each R² is independently selected from a naturally or non-naturally occurring nucleobase, which, when taken together, forms a targeting sequence of 13-28 bases in length. In an embodiment, each R² is independently selected from a naturally or non-naturally occurring nucleobase, which, when taken together, forms a targeting sequence of 13-27 bases in length. In an embodiment, each R² is independently selected from a naturally or non-naturally occurring nucleobase, which, when taken together, forms a targeting sequence of 13-26 bases in length. In an embodiment, each R² is independently selected from a naturally or non-naturally occurring nucleobase, which, when taken together, forms a targeting sequence of 13-25 bases in length. In an embodiment, each R² is independently selected from a naturally or non-naturally occurring nucleobase, which, when taken together, forms a targeting sequence of 13-24 bases in length. In an embodiment, each R² is independently selected from a naturally or non-naturally occurring nucleobase, which, when taken together, forms a targeting sequence of 13-23 bases in length. In an embodiment, each R² is independently selected from a naturally or non-naturally occurring nucleobase, which, when taken together, forms a targeting sequence of 13-22 bases in length. In an embodiment, each R² is independently selected from a naturally or non-naturally occurring nucleobase, which, when taken together, forms a targeting sequence of 13-21 bases in length. In an embodiment, each R² is independently selected from a naturally or non-naturally occurring nucleobase, which, when taken together, forms a targeting sequence or 13-20 bases in length. In an embodiment, each R² is independently selected from a naturally or non-naturally occurring nucleobase, which, when taken together, forms a targeting sequence or 13-19 bases in length. In an embodiment, each R² is independently selected from a naturally or non-naturally occurring nucleobase, which, when taken together, forms a targeting sequence of 13-18 bases in length. In an embodiment, each R² is independently selected from a naturally or non-naturally occurring nucleobase, which, when taken together, forms a targeting sequence of 13-17 bases in length. In an embodiment, each R² is independently selected from a naturally or non-naturally occurring nucleobase, which, when taken together, forms a targeting sequence of 13-16 bases in length. In an embodiment, each R² is independently selected from a naturally or non-naturally occurring nucleobase, which, when taken together, forms a targeting sequence of 13-15 bases in length. In an embodiment, each R² is independently selected from a naturally or non-naturally occurring nucleobase, which, when taken together, forms a targeting sequence of 13-14 bases in length.

In another embodiment, each R² is independently selected from a naturally or non-naturally occurring nucleobase, which, when taken together, forms a targeting sequence of 14-30 bases in length. In an embodiment, each R² is independently selected from a naturally or non-naturally occurring nucleobase, which, when taken together, forms a targeting sequence of 14-29 bases in length. In an embodiment, each R² is independently selected from a naturally or non-naturally occurring nucleobase, which, when taken together, forms a targeting sequence of 14-28 bases in length. In an embodiment, each R² is independently selected from a naturally or non-naturally occurring nucleobase, which, when taken together, forms a targeting sequence of 14-27 bases in length. In an embodiment, each R² is independently selected from a naturally or non-naturally occurring nucleobase, which, when taken together, forms a targeting sequence of 14-26 bases in length. In an embodiment, each R² is independently selected from a naturally or non-naturally occurring nucleobase, which, when taken together, forms a targeting sequence of 14-25 bases in length. In an embodiment, each R² is independently selected from a naturally or non-naturally occurring nucleobase, which, when taken together, forms a targeting sequence of 14-24 bases in length. In an embodiment, each R² is independently selected from a naturally or non-naturally occurring nucleobase, which, when taken together, forms a targeting sequence of 14-23 bases in length. In an embodiment, each R² is independently selected from a naturally or non-naturally occurring nucleobase, which, when taken together, forms a targeting sequence of 14-22 bases in length. In an embodiment, each R² is independently selected from a naturally or non-naturally occurring nucleobase, which, when taken together, forms a targeting sequence of 14-21 bases in length. In an embodiment, each R² is independently selected from a naturally or non-naturally occurring nucleobase, which, when taken together, forms a targeting sequence of 14-20 bases in length. In an embodiment, each R² is independently selected from a naturally or non-naturally occurring nucleobase, which, when taken together, forms a targeting sequence of 14-19 bases in length. In an embodiment, each R² is independently selected from a naturally or non-naturally occurring nucleobase, which, when taken together, forms a targeting sequence of 14-18 bases in length. In an embodiment, each R² is independently selected from a naturally or non-naturally occurring nucleobase, which, when taken together, forms a targeting sequence of 14-17 bases in length. In an embodiment, each R² is independently selected from a naturally or non-naturally occurring nucleobase, which, when taken together, forms a targeting sequence of 14-16 bases in length. In an embodiment, each R² is independently selected from a naturally or non-naturally occurring nucleobase, which, when taken together, forms a targeting sequence of 14-15 bases in length.

In another embodiment, each R² is independently selected from a naturally or non-naturally occurring nucleobase, which, when taken together, forms a targeting sequence of 15-30 bases in length. In an embodiment, each R² is independently selected from a naturally or non-naturally occurring nucleobase, which, when taken together, forms a targeting sequence of 15-29 bases in length. In an embodiment, each R² is independently selected from a naturally or non-naturally occurring nucleobase, which, when taken together, forms a targeting sequence of 15-28 bases in length. In an embodiment, each R² is independently selected from a naturally or non-naturally occurring nucleobase, which, when taken together, forms a targeting sequence of 15-27 bases in length. In an embodiment, each R² is independently selected from a naturally or non-naturally occurring nucleobase, which, when taken together, forms a targeting sequence of 15-26 bases in length. In an embodiment, each R² is independently selected from a naturally or non-naturally occurring nucleobase, which, when taken together, forms a targeting sequence of 15-25 bases in length. In an embodiment, each R² is independently selected from a naturally or non-naturally occurring nucleobase, which, when taken together, forms a targeting sequence of 15-24 bases in length. In an embodiment, each R² is independently selected from a naturally or non-naturally occurring nucleobase, which, when taken together, forms a targeting sequence of 15-23 bases in length. In an embodiment, each R² is independently selected from a naturally or non-naturally occurring nucleobase, which, when taken together, forms a targeting sequence of 15-22 bases in length. In an embodiment, each R² is independently selected from a naturally or non-naturally occurring nucleobase, which, when taken together, forms a targeting sequence of 15-21 bases in length. In an embodiment, each R² is independently selected from a naturally or non-naturally occurring nucleobase, which, when taken together, forms a targeting sequence of 15-20 bases in length. In an embodiment, each R² is independently selected from a naturally or non-naturally occurring nucleobase, which, when taken together, forms a targeting sequence of 15-19 bases in length. In an embodiment, each R² is independently selected from a naturally or non-naturally occurring nucleobase, which, when taken together, forms a targeting sequence of 15-18 bases in length. In an embodiment, each R² is independently selected from a naturally or non-naturally occurring nucleobase, which, when taken together, forms a targeting sequence of 15-17 bases in length. In an embodiment, each R² is independently selected from a naturally or non-naturally occurring nucleobase, which, when taken together, forms a targeting sequence of 15-16 bases in length.

In another embodiment, each R² is independently selected from a naturally or non-naturally occurring nucleobase, which, when taken together, forms a targeting sequence of 16-30 bases in length. In an embodiment, each R² is independently selected from a naturally or non-naturally occurring nucleobase, which, when taken together, forms a targeting sequence of 16-29 bases in length. In an embodiment, each R² is independently selected from a naturally or non-naturally occurring nucleobase, which, when taken together, forms a targeting sequence of 16-28 bases in length. In an embodiment, each R² is independently selected from a naturally or non-naturally occurring nucleobase, which, when taken together, forms a targeting sequence of 16-27 bases in length. In an embodiment, each R² is independently selected from a naturally or non-naturally occurring nucleobase, which, when taken together, forms a targeting sequence of 16-26 bases in length. In an embodiment, each R² is independently selected from a naturally or non-naturally occurring nucleobase, which, when taken together, forms a targeting sequence of 16-25 bases in length. In an embodiment, each R² is independently selected from a naturally or non-naturally occurring nucleobase, which, when taken together, forms a targeting sequence of 16-24 bases in length. In an embodiment, each R² is independently selected from a naturally or non-naturally occurring nucleobase, which, when taken together, forms a targeting sequence of 16-23 bases in length. In an embodiment, each R² is independently selected from a naturally or non-naturally occurring nucleobase, which, when taken together, forms a targeting sequence of 16-22 bases in length. In an embodiment, each R² is independently selected from a naturally or non-naturally occurring nucleobase, which, when taken together, forms a targeting sequence of 16-21 bases in length. In an embodiment, each R² is independently selected from a naturally or non-naturally occurring nucleobase, which, when taken together, forms a targeting sequence of 16-20 bases in length. In an embodiment, each R² is independently selected from a naturally or non-naturally occurring nucleobase, which, when taken together, forms a targeting sequence of 16-19 bases in length. In an embodiment, each R² is independently selected from a naturally or non-naturally occurring nucleobase, which, when taken together, forms a targeting sequence of 16-18 bases in length. In an embodiment, each R² is independently selected from a naturally or non-naturally occurring nucleobase, which, when taken together, forms a targeting sequence of 16-17 bases in length.

In another embodiment, each R² is independently selected from a naturally or non-naturally occurring nucleobase, which, when taken together, forms a targeting sequence of 17-30 bases in length. In an embodiment, each R² is independently selected from a naturally or non-naturally occurring nucleobase, which, when taken together, forms a targeting sequence of 17-29 bases in length. In an embodiment, each R² is independently selected from a naturally or non-naturally occurring nucleobase, which, when taken together, forms a targeting sequence of 17-28 bases in length. In an embodiment, each R² is independently selected from a naturally or non-naturally occurring nucleobase, which, when taken together, forms a targeting sequence of 17-27 bases in length. In an embodiment, each R² is independently selected from a naturally or non-naturally occurring nucleobase, which, when taken together, forms a targeting sequence of 17-26 bases in length. In an embodiment, each R² is independently selected from a naturally or non-naturally occurring nucleobase, which, when taken together, forms a targeting sequence of 17-25 bases in length. In an embodiment, each R² is independently selected from a naturally or non-naturally occurring nucleobase, which, when taken together, forms a targeting sequence of 17-24 bases in length. In an embodiment, each R² is independently selected from a naturally or non-naturally occurring nucleobase, which, when taken together, forms a targeting sequence of 17-23 bases in length. In an embodiment, each R² is independently selected from a naturally or non-naturally occurring nucleobase, which, when taken together, forms a targeting sequence of 17-22 bases in length. In an embodiment, each R² is independently selected from a naturally or non-naturally occurring nucleobase, which, when taken together, forms a targeting sequence of 17-21 bases in length. In an embodiment, each R² is independently selected from a naturally or non-naturally occurring nucleobase, which, when taken together, forms a targeting sequence of 17-20 bases in length. In an embodiment, each R² is independently selected from a naturally or non-naturally occurring nucleobase, which, when taken together, forms a targeting sequence of 17-19 bases in length. In an embodiment, each R² is independently selected from a naturally or non-naturally occurring nucleobase, which, when taken together, forms a targeting sequence of 17-18 bases in length.

In another embodiment, each R² is independently selected from a naturally or non-naturally occurring nucleobase, which, when taken together, forms a targeting sequence of 18-30 bases in length. In an embodiment, each R² is independently selected from a naturally or non-naturally occurring nucleobase, which, when taken together, forms a targeting sequence of 18-29 bases in length. In an embodiment, each R² is independently selected from a naturally or non-naturally occurring nucleobase, which, when taken together, forms a targeting sequence of 18-28 bases in length. In an embodiment, each R² is independently selected from a naturally or non-naturally occurring nucleobase, which, when taken together, forms a targeting sequence of 18-27 bases in length. In an embodiment, each R² is independently selected from a naturally or non-naturally occurring nucleobase, which, when taken together, forms a targeting sequence of 18-26 bases in length. In an embodiment, each R² is independently selected from a naturally or non-naturally occurring nucleobase, which, when taken together, forms a targeting sequence of 18-25 bases in length. In an embodiment, each R² is independently selected from a naturally or non-naturally occurring nucleobase, which, when taken together, forms a targeting sequence of 18-24 bases in length. In an embodiment, each R² is independently selected from a naturally or non-naturally occurring nucleobase, which, when taken together, forms a targeting sequence of 18-23 bases in length. In an embodiment, each R² is independently selected from a naturally or non-naturally occurring nucleobase, which, when taken together, forms a targeting sequence of 18-22 bases in length. In an embodiment, each R² is independently selected from a naturally or non-naturally occurring nucleobase, which, when taken together, forms a targeting sequence of 18-21 bases in length. In an embodiment, each R² is independently selected from a naturally or non-naturally occurring nucleobase, which, when taken together, forms a targeting sequence of 18-20 bases in length. In an embodiment, each R² is independently selected from a naturally or non-naturally occurring nucleobase, which, when taken together, forms a targeting sequence of 18-19 bases in length.

In another embodiment, each R² is independently selected from a naturally or non-naturally occurring nucleobase, which, when taken together, forms a targeting sequence of 19-30 bases in length. In an embodiment, each R² is independently selected from a naturally or non-naturally occurring nucleobase, which, when taken together, forms a targeting sequence of 19-29 bases in length. In an embodiment, each R² is independently selected from a naturally or non-naturally occurring nucleobase, which, when taken together, forms a targeting sequence of 19-28 bases in length. In an embodiment, each R² is independently selected from a naturally or non-naturally occurring nucleobase, which, when taken together, forms a targeting sequence of 19-27 bases in length. In an embodiment, each R² is independently selected from a naturally or non-naturally occurring nucleobase, which, when taken together, forms a targeting sequence of 19-26 bases in length. In an embodiment, each R² is independently selected from a naturally or non-naturally occurring nucleobase, which, when taken together, forms a targeting sequence of 19-25 bases in length. In an embodiment, each R² is independently selected from a naturally or non-naturally occurring nucleobase, which, when taken together, forms a targeting sequence of 19-24 bases in length. In an embodiment, each R² is independently selected from a naturally or non-naturally occurring nucleobase, which, when taken together, forms a targeting sequence of 19-23 bases in length. In an embodiment, each R² is independently selected from a naturally or non-naturally occurring nucleobase, which, when taken together, forms a targeting sequence of 19-22 bases in length. In an embodiment, each R² is independently selected from a naturally or non-naturally occurring nucleobase, which, when taken together, forms a targeting sequence of 19-21 bases in length. In an embodiment, each R² is independently selected from a naturally or non-naturally occurring nucleobase, which, when taken together, forms a targeting sequence of 19-20 bases in length.

In another embodiment, each R² is independently selected from a naturally or non-naturally occurring nucleobase, which, when taken together, forms a targeting sequence of 20-30 bases in length. In an embodiment, each R² is independently selected from a naturally or non-naturally occurring nucleobase, which, when taken together, forms a targeting sequence of 20-29 bases in length. In an embodiment, each R² is independently selected from a naturally or non-naturally occurring nucleobase, which, when taken together, forms a targeting sequence of 20-28 bases in length. In an embodiment, each R² is independently selected from a naturally or non-naturally occurring nucleobase, which, when taken together, forms a targeting sequence of 20-27 bases in length. In an embodiment, each R² is independently selected from a naturally or non-naturally occurring nucleobase, which, when taken together, forms a targeting sequence of 20-26 bases in length. In an embodiment, each R² is independently selected from a naturally or non-naturally occurring nucleobase, which, when taken together, forms a targeting sequence of 20-25 bases in length. In an embodiment, each R² is independently selected from a naturally or non-naturally occurring nucleobase, which, when taken together, forms a targeting sequence of 20-24 bases in length. In an embodiment, each R² is independently selected from a naturally or non-naturally occurring nucleobase, which, when taken together, forms a targeting sequence of 20-23 bases in length. In an embodiment, each R² is independently selected from a naturally or non-naturally occurring nucleobase, which, when taken together, forms a targeting sequence of 20-22 bases in length. In an embodiment, each R² is independently selected from a naturally or non-naturally occurring nucleobase, which, when taken together, forms a targeting sequence of 20-21 bases in length.

In another embodiment, each R² is independently selected from a naturally or non-naturally occurring nucleobase, which, when taken together, forms a targeting sequence of 21-30 bases in length. In an embodiment, each R² is independently selected from a naturally or non-naturally occurring nucleobase, which, when taken together, forms a targeting sequence of 21-29 bases in length. In an embodiment, each R² is independently selected from a naturally or non-naturally occurring nucleobase, which, when taken together, forms a targeting sequence of 21-28 bases in length. In an embodiment, each R² is independently selected from a naturally or non-naturally occurring nucleobase, which, when taken together, forms a targeting sequence of 21-27 bases in length. In an embodiment, each R² is independently selected from a naturally or non-naturally occurring nucleobase, which, when taken together, forms a targeting sequence of 21-26 bases in length. In an embodiment, each R² is independently selected from a naturally or non-naturally occurring nucleobase, which, when taken together, forms a targeting sequence of 21-25 bases in length. In an embodiment, each R² is independently selected from a naturally or non-naturally occurring nucleobase, which, when taken together, forms a targeting sequence of 21-24 bases in length. In an embodiment, each R² is independently selected from a naturally or non-naturally occurring nucleobase, which, when taken together, forms a targeting sequence of 21-23 bases in length. In an embodiment, each R² is independently selected from a naturally or non-naturally occurring nucleobase, which, when taken together, forms a targeting sequence of 21-22 bases in length.

In another embodiment, each R² is independently selected from a naturally or non-naturally occurring nucleobase, which, when taken together, forms a targeting sequence of 22-30 bases in length. In an embodiment, each R² is independently selected from a naturally or non-naturally occurring nucleobase, which, when taken together, forms a targeting sequence of 22-29 bases in length. In an embodiment, each R² is independently selected from a naturally or non-naturally occurring nucleobase, which, when taken together, forms a targeting sequence of 22-28 bases in length. In an embodiment, each R² is independently selected from a naturally or non-naturally occurring nucleobase, which, when taken together, forms a targeting sequence of 22-27 bases in length. In an embodiment, each R² is independently selected from a naturally or non-naturally occurring nucleobase, which, when taken together, forms a targeting sequence of 22-26 bases in length. In an embodiment, each R² is independently selected from a naturally or non-naturally occurring nucleobase, which, when taken together, forms a targeting sequence of 22-25 bases in length. In an embodiment, each R² is independently selected from a naturally or non-naturally occurring nucleobase, which, when taken together, forms a targeting sequence of 22-24 bases in length. In an embodiment, each R² is independently selected from a naturally or non-naturally occurring nucleobase, which, when taken together, forms a targeting sequence of 22-23 bases in length.

In another embodiment, each R² is independently selected from a naturally or non-naturally occurring nucleobase, which, when taken together, forms a targeting sequence of 23-30 bases in length. In an embodiment, each R² is independently selected from a naturally or non-naturally occurring nucleobase, which, when taken together, forms a targeting sequence of 23-29 bases in length. In an embodiment, each R² is independently selected from a naturally or non-naturally occurring nucleobase, which, when taken together, forms a targeting sequence of 23-28 bases in length. In an embodiment, each R² is independently selected from a naturally or non-naturally occurring nucleobase, which, when taken together, forms a targeting sequence of 23-27 bases in length. In an embodiment, each R² is independently selected from a naturally or non-naturally occurring nucleobase, which, when taken together, forms a targeting sequence of 23-26 bases in length. In an embodiment, each R² is independently selected from a naturally or non-naturally occurring nucleobase, which, when taken together, forms a targeting sequence of 23-25 bases in length. In an embodiment, each R² is independently selected from a naturally or non-naturally occurring nucleobase, which, when taken together, forms a targeting sequence of 23-24 bases in length.

In another embodiment, each R² is independently selected from a naturally or non-naturally occurring nucleobase, which, when taken together, forms a targeting sequence of 24-30 bases in length. In an embodiment, each R² is independently selected from a naturally or non-naturally occurring nucleobase, which, when taken together, forms a targeting sequence of 24-29 bases in length. In an embodiment, each R² is independently selected from a naturally or non-naturally occurring nucleobase, which, when taken together, forms a targeting sequence of 24-28 bases in length. In an embodiment, each R² is independently selected from a naturally or non-naturally occurring nucleobase, which, when taken together, forms a targeting sequence of 24-27 bases in length. In an embodiment, each R² is independently selected from a naturally or non-naturally occurring nucleobase, which, when taken together, forms a targeting sequence of 24-26 bases in length. In an embodiment, each R² is independently selected from a naturally or non-naturally occurring nucleobase, which, when taken together, forms a targeting sequence of 24-25 bases in length.

In another embodiment, each R² is independently selected from a naturally or non-naturally occurring nucleobase, which, when taken together, forms a targeting sequence of 25-30 bases in length. In an embodiment, each R² is independently selected from a naturally or non-naturally occurring nucleobase, which, when taken together, forms a targeting sequence of 25-29 bases in length. In an embodiment, each R² is independently selected from a naturally or non-naturally occurring nucleobase, which, when taken together, forms a targeting sequence of 25-28 bases in length. In an embodiment, each R² is independently selected from a naturally or non-naturally occurring nucleobase, which, when taken together, forms a targeting sequence of 25-27 bases in length. In an embodiment, each R² is independently selected from a naturally or non-naturally occurring nucleobase, which, when taken together, forms a targeting sequence of 25-26 bases in length.

In another embodiment, each R² is independently selected from a naturally or non-naturally occurring nucleobase, which, when taken together, forms a targeting sequence of 26-30 bases in length. In an embodiment, each R² is independently selected from a naturally or non-naturally occurring nucleobase, which, when taken together, forms a targeting sequence of 26-29 bases in length. In an embodiment, each R² is independently selected from a naturally or non-naturally occurring nucleobase, which, when taken together, forms a targeting sequence of 26-28 bases in length. In an embodiment, each R² is independently selected from a naturally or non-naturally occurring nucleobase, which, when taken together, forms a targeting sequence of 26-27 bases in length.

In another embodiment, each R² is independently selected from a naturally or non-naturally occurring nucleobase, which, when taken together, forms a targeting sequence of 27-30 bases in length. In an embodiment, each R² is independently selected from a naturally or non-naturally occurring nucleobase, which, when taken together, forms a targeting sequence of 27-29 bases in length. In an embodiment, each R² is independently selected from a naturally or non-naturally occurring nucleobase, which, when taken together, forms a targeting sequence of 27-28 bases in length.

In another embodiment, each R² is independently selected from a naturally or non-naturally occurring nucleobase, which, when taken together, forms a targeting sequence of 28-30 bases in length. In an embodiment, each R² is independently selected from a naturally or non-naturally occurring nucleobase, which, when taken together, forms a targeting sequence of 28-29 bases in length. In another embodiment, each R² is independently selected from a naturally or non-naturally occurring nucleobase, which, when taken together, forms a targeting sequence of 28-30 bases in length. In an embodiment, each R² is independently selected from a naturally or non-naturally occurring nucleobase, which, when taken together, forms a targeting sequence of 29-30 bases in length.

In another embodiment, each R² is independently selected from a naturally or non-naturally occurring nucleobase, which, when taken together, forms a targeting sequence of 13 bases in length.

In another embodiment, each R² is independently selected from a naturally or non-naturally occurring nucleobase, which, when taken together, forms a targeting sequence of 14 bases in length. In another embodiment, each R² is independently selected from a naturally or non-naturally occurring nucleobase, which, when taken together, forms a targeting sequence of 15 bases in length. In another embodiment, each R² is independently selected from a naturally or non-naturally occurring nucleobase, which, when taken together, forms a targeting sequence of 16 bases in length. In another embodiment, each R² is independently selected from a naturally or non-naturally occurring nucleobase, which, when taken together, forms a targeting sequence of 17 bases in length. In another embodiment, each R² is independently selected from a naturally or non-naturally occurring nucleobase, which, when taken together, forms a targeting sequence of 18 bases in length. In another embodiment, each R² is independently selected from a naturally or non-naturally occurring nucleobase, which, when taken together, forms a targeting sequence of 19 bases in length. In another embodiment, each R² is independently selected from a naturally or non-naturally occurring nucleobase, which, when taken together, forms a targeting sequence of 20 bases in length. In another embodiment, each R² is independently selected from a naturally or non-naturally occurring nucleobase, which, when taken together, forms a targeting sequence of 21 bases in length. In another embodiment, each R² is independently selected from a naturally or non-naturally occurring nucleobase, which, when taken together, forms a targeting sequence of 22 bases in length. In another embodiment, each R² is independently selected from a naturally or non-naturally occurring nucleobase, which, when taken together, forms a targeting sequence of 23 bases in length. In another embodiment, each R² is independently selected from a naturally or non-naturally occurring nucleobase, which, when taken together, forms a targeting sequence of 24 bases in length. In another embodiment, each R² is independently selected from a naturally or non-naturally occurring nucleobase, which, when taken together, forms a targeting sequence of 25 bases in length. In another embodiment, each R² is independently selected from a naturally or non-naturally occurring nucleobase, which, when taken together, forms a targeting sequence of 26 bases in length. In another embodiment, each R² is independently selected from a naturally or non-naturally occurring nucleobase, which, when taken together, forms a targeting sequence of 27 bases in length. In another embodiment, each R² is independently selected from a naturally or non-naturally occurring nucleobase, which, when taken together, forms a targeting sequence of 28 bases in length. In another embodiment, each R² is independently selected from a naturally or non-naturally occurring nucleobase, which, when taken together, forms a targeting sequence of 29 bases in length. In another embodiment, each R² is independently selected from a naturally or non-naturally occurring nucleobase, which, when taken together, forms a targeting sequence of 30 bases in length. In certain embodiments of the antisense oligomer, or pharmaceutically acceptable salt thereof, of structural Formula (IA), the salt is an HCl salt.

### Formula (II)

In some embodiments, provided herein is an antisense oligomer of Formula (I) which is an oligomer of Formula (II):

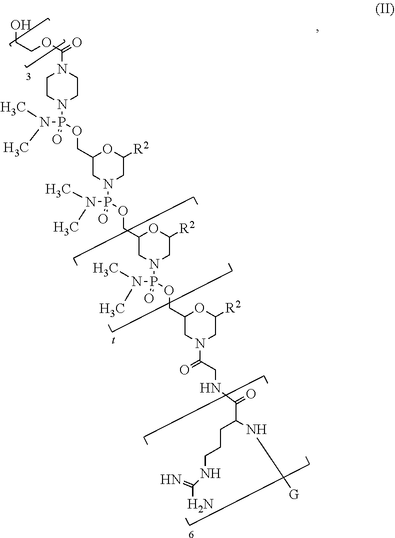

or a pharmaceutically acceptable salt thereof,
wherein:
each R² is independently selected from a naturally or non-naturally occurring nucleobase, which, when taken together, forms a targeting sequence of 13 to 30 bases, that is complementary to a target region within a pre-mRNA of the human uromodulin (UMOD) gene (SEQ ID NO: 1) wherein the target region is an intron/exon junction or an exon internal region of the human UMOD gene pre-mRNA;
t is 11-28; and
G is selected from —H, —C(O)C1-6-alkyl, benzoyl, and stearoyl.

In some embodiments, t of Formula (II) is 11, 12, 13, 14, 15, 16, 17, 18, 19, 20, 21, 22, 23, 24, 25, 26, 27, or 28. In some embodiments, each R² of Formula (II) taken together forms a targeting sequence of 13, 14, 15, 16, 17, 18, 19, 20, 21, 22, 23, 24, 25, 26, 27, 28, 29, or 30 bases. In some embodiments, each R² of Formula (II) taken together forms a targeting sequence of 18-27 bases. In some embodiments, each R² of Formula (II) taken together forms a targeting sequence of 20-25 bases. In certain embodiments of Formula (II), the pharmaceutically acceptable salt is an HCl salt.

In an embodiment, t of Formula (II) is an integer from 11 to 28. In an embodiment, t of Formula (II) Is at least 11. In an embodiment, t of Formula (II) is up to 28.

In an embodiment, t is 11-28. In an embodiment, t is 11-27. In an embodiment, t is 11-26. In an embodiment, t is 11-25. In an embodiment, t is 11-24. In an embodiment, t is 11-23. In an embodiment, t is 11-22. In an embodiment, t is 11-21. In an embodiment, t is 11-20. In an embodiment, t is 11-19. In an embodiment, t is 11-18. In an embodiment, t is 11-17. In an embodiment, t is 11-16. In an embodiment, t is 11-15. In an embodiment, t is 11-14. In an embodiment, t is 11-13. In an embodiment, t is 11-12. In an embodiment, t is 12-28. In an embodiment, t is 12-27. In an embodiment, t is 12-26. In an embodiment, t is 12-25. In an embodiment, t is 12-24. In an embodiment, t is 12-23. In an embodiment, t is 12-22. In an embodiment, t is 12-21. In an embodiment, t is 12-20. In an embodiment, t is 12-19. In an embodiment, t is 12-18. In an embodiment, t is 12-17. In an embodiment, t is 12-16. In an embodiment, t is 12-15. In an embodiment, t is 12-14. In an embodiment, t is 12-13. In an embodiment, t is 13-28. In an embodiment, t is 13-27. In an embodiment, t is 13-26. In an embodiment, t is 13-25. In an embodiment, t is 13-24. In an embodiment, t is 13-23. In an embodiment, t is 13-22. In an embodiment, t is 13-21. In an embodiment, t is 13-20. In an embodiment, t is 13-19. In an embodiment, t is 13-18. In an embodiment, t is 13-17. In an embodiment, t is 13-16. In an embodiment, t is 13-15. In an embodiment, t is 13-14. In an embodiment, t is 14-28. In an embodiment, t is 14-27. In an embodiment, t is 14-26. In an embodiment, t is 14-25. In an embodiment, t is 14-24. In an embodiment, t is 14-23. In an embodiment, t is 14-22. In an embodiment, t is 14-21. In an embodiment, t is 14-20. In an embodiment, t is 14-19. In an embodiment, t is 14-18. In an embodiment, t is 14-17. In an embodiment, t is 14-16. In an embodiment, t is 14-15. In an embodiment, t is 15-28. In an embodiment, t is 15-27. In an embodiment, t is 15-26. In an embodiment, t is 15-25. In an embodiment, t is 15-24. In an embodiment, t is 15-23. In an embodiment, t is 15-22. In an embodiment, t is 15-21. In an embodiment, t is 15-20. In an embodiment, t is 15-19. In an embodiment, t is 15-18. In an embodiment, t is 15-17. In an embodiment, t is 15-16. In an embodiment, t is 16-28. In an embodiment, t is 16-27. In an embodiment, t is 16-26. In an embodiment, t is 16-25. In an embodiment, t is 16-24. In an embodiment, t is 16-23. In an embodiment, t is 16-22. In an embodiment, t is 16-21. In an embodiment, t is 16-20. In an embodiment, t is 16-19. In an embodiment, t is 16-18. In an embodiment, t is 16-17. In an embodiment, t is 17-28. In an embodiment, t is 17-27. In an embodiment, t is 17-26. In an embodiment, t is 17-25. In an embodiment, t is 17-24. In an embodiment, t is 17-23. In an embodiment, t is 17-22. In an embodiment, t is 17-21. In an embodiment, t is 17-20. In an embodiment, t is 17-19. In an embodiment, t is 17-18. In an embodiment, t is 18-28. In an embodiment, t is 18-27. In an embodiment, t is 18-26. In an embodiment, t is 18-25. In an embodiment, t is 18-24. In an embodiment, t is 18-23. In an embodiment, t is 18-22. In an embodiment, t is 18-21. In an embodiment, t is 18-20. In an embodiment, t is 18-19. In an embodiment, t is 19-28. In an embodiment, t is 19-27. In an embodiment, t is 19-26. In an embodiment, t is 19-25. In an embodiment, t is 19-24. In an embodiment, t is 19-23. In an embodiment, t is 19-22. In an embodiment, t is 19-21. In an embodiment, t is 19-20. In an embodiment, t is 20-28. In an embodiment, t is 20-27. In an embodiment, t is 20-26. In an embodiment, t is 20-25. In an embodiment, t is 20-24. In an embodiment, t is 20-23. In an embodiment, t is 20-22. In an embodiment, t is 20-21. In an embodiment, t is 21-28. In an embodiment, t is 21-27. In an embodiment, t is 21-26. In an embodiment, t is 21-25. In an embodiment, t is 21-24. In an embodiment, t is 21-23. In an embodiment, t is 21-22. In an embodiment, t is 22-28. In an embodiment, t is 22-27. In an embodiment, t is 22-26. In an embodiment, t is 22-25. In an embodiment, t is 22-24. In an embodiment, t is 22-23. In an embodiment, t is 23-28. In an embodiment, t is 23-27. In an embodiment, t is 23-26. In an embodiment, t is 23-25. In an embodiment, t is 23-24. In an embodiment, t is 24-28. In an embodiment, t is 24-27. In an embodiment, t is 24-26. In an embodiment, t is 24-25. In an embodiment, t is 25-28. In an embodiment, t is 25-27. In an embodiment, t is 25-26. In another embodiment, t is 26-30. In an embodiment, t is 26-29. In an embodiment, t is 26-28. In an embodiment, t is 26-27. In an embodiment, t is 27-28.

In an embodiment, t is 11. In an embodiment, t is 12. In an embodiment, t is 13. In an embodiment, t is 14. In an embodiment, t is 15. In an embodiment, t is 16. In an embodiment, t is 17. In an embodiment, t is 18. In an embodiment, t is 19. In an embodiment, t is 20. In an embodiment, t is 21. In an embodiment, t is 22. In an embodiment, t is 23. In an embodiment, t is 24. In an embodiment, t is 25. In an embodiment, t is 26. In an embodiment, t is 27. In an embodiment, t is 28.

In an embodiment, each R² is independently selected from a naturally or non-naturally occurring nucleobase, which, when taken together, forms a targeting sequence of from 13 to 30 bases in length. In an embodiment, each R² is independently selected from a naturally or non-naturally occurring nucleobase, which, when taken together, forms a targeting sequence at least 13 bases in length. In an embodiment, each R² is independently selected from a naturally or non-naturally occurring nucleobase, which, when taken together, forms a targeting sequence of up to 30 bases in length.

In an embodiment, each R² is independently selected from a naturally or non-naturally occurring nucleobase, which, when taken together, forms a targeting sequence of 13-29 bases in length. In an embodiment, each R² is independently selected from a naturally or non-naturally occurring nucleobase, which, when taken together, forms a targeting sequence of 13-28 bases in length. In an embodiment, each R² is independently selected from a naturally or non-naturally occurring nucleobase, which, when taken together, forms a targeting sequence of 13-27 bases in length. In an embodiment, each R² is independently selected from a naturally or non-naturally occurring nucleobase, which, when taken together, forms a targeting sequence of 13-26 bases in length. In an embodiment, each R² is independently selected from a naturally or non-naturally occurring nucleobase, which, when taken together, forms a targeting sequence of 13-25 bases in length. In an embodiment, each R² is independently selected from a naturally or non-naturally occurring nucleobase, which, when taken together, forms a targeting sequence of 13-24 bases in length. In an embodiment, each R² is independently selected from a naturally or non-naturally occurring nucleobase, which, when taken together, forms a targeting sequence of 13-23 bases in length. In an embodiment, each R² is independently selected from a naturally or non-naturally occurring nucleobase, which, when taken together, forms a targeting sequence of 13-22 bases in length. In an embodiment, each R² is independently selected from a naturally or non-naturally occurring nucleobase, which, when taken together, forms a targeting sequence of 13-21 bases in length. In an embodiment, each R² is independently selected from a naturally or non-naturally occurring nucleobase, which, when taken together, forms a targeting sequence or 13-20 bases in length. In an embodiment, each R² is independently selected from a naturally or non-naturally occurring nucleobase, which, when taken together, forms a targeting sequence or 13-19 bases in length. In an embodiment, each R² is independently selected from a naturally or non-naturally occurring nucleobase, which, when taken together, forms a targeting sequence of 13-18 bases in length. In an embodiment, each R² is independently selected from a naturally or non-naturally occurring nucleobase, which, when taken together, forms a targeting sequence of 13-17 bases in length. In an embodiment, each R² is independently selected from a naturally or non-naturally occurring nucleobase, which, when taken together, forms a targeting sequence of 13-16 bases in length. In an embodiment, each R² is independently selected from a naturally or non-naturally occurring nucleobase, which, when taken together, forms a targeting sequence of 13-15 bases in length. In an embodiment, each R² is independently selected from a naturally or non-naturally occurring nucleobase, which, when taken together, forms a targeting sequence of 13-14 bases in length.

In another embodiment, each R² is independently selected from a naturally or non-naturally occurring nucleobase, which, when taken together, forms a targeting sequence of 14-30 bases in length. In an embodiment, each R² is independently selected from a naturally or non-naturally occurring nucleobase, which, when taken together, forms a targeting sequence of 14-29 bases in length. In an embodiment, each R² is independently selected from a naturally or non-naturally occurring nucleobase, which, when taken together, forms a targeting sequence of 14-28 bases in length. In an embodiment, each R² is independently selected from a naturally or non-naturally occurring nucleobase, which, when taken together, forms a targeting sequence of 14-27 bases in length. In an embodiment, each R² is independently selected from a naturally or non-naturally occurring nucleobase, which, when taken together, forms a targeting sequence of 14-26 bases in length. In an embodiment, each R² is independently selected from a naturally or non-naturally occurring nucleobase, which, when taken together, forms a targeting sequence of 14-25 bases in length. In an embodiment, each R² is independently selected from a naturally or non-naturally occurring nucleobase, which, when taken together, forms a targeting sequence of 14-24 bases in length. In an embodiment, each R² is independently selected from a naturally or non-naturally occurring nucleobase, which, when taken together, forms a targeting sequence of 14-23 bases in length. In an embodiment, each R² is independently selected from a naturally or non-naturally occurring nucleobase, which, when taken together, forms a targeting sequence of 14-22 bases in length. In an embodiment, each R² is independently selected from a naturally or non-naturally occurring nucleobase, which, when taken together, forms a targeting sequence of 14-21 bases in length. In an embodiment, each R² is independently selected from a naturally or non-naturally occurring nucleobase, which, when taken together, forms a targeting sequence of 14-20 bases in length. In an embodiment, each R² is independently selected from a naturally or non-naturally occurring nucleobase, which, when taken together, forms a targeting sequence of 14-19 bases in length. In an embodiment, each R² is independently selected from a naturally or non-naturally occurring nucleobase, which, when taken together, forms a targeting sequence of 14-18 bases in length. In an embodiment, each R² is independently selected from a naturally or non-naturally occurring nucleobase, which, when taken together, forms a targeting sequence of 14-17 bases in length. In an embodiment, each R² is independently selected from a naturally or non-naturally occurring nucleobase, which, when taken together, forms a targeting sequence of 14-16 bases in length. In an embodiment, each R² is independently selected from a naturally or non-naturally occurring nucleobase, which, when taken together, forms a targeting sequence of 14-15 bases in length.

In another embodiment, each R² is independently selected from a naturally or non-naturally occurring nucleobase, which, when taken together, forms a targeting sequence of 15-30 bases in length. In an embodiment, each R² is independently selected from a naturally or non-naturally occurring nucleobase, which, when taken together, forms a targeting sequence of 15-29 bases in length. In an embodiment, each R² is independently selected from a naturally or non-naturally occurring nucleobase, which, when taken together, forms a targeting sequence of 15-28 bases in length. In an embodiment, each R² is independently selected from a naturally or non-naturally occurring nucleobase, which, when taken together, forms a targeting sequence of 15-27 bases in length. In an embodiment, each R² is independently selected from a naturally or non-naturally occurring nucleobase, which, when taken together, forms a targeting sequence of 15-26 bases in length. In an embodiment, each R² is independently selected from a naturally or non-naturally occurring nucleobase, which, when taken together, forms a targeting sequence of 15-25 bases in length. In an embodiment, each R² is independently selected from a naturally or non-naturally occurring nucleobase, which, when taken together, forms a targeting sequence of 15-24 bases in length. In an embodiment, each R² is independently selected from a naturally or non-naturally occurring nucleobase, which, when taken together, forms a targeting sequence of 15-23 bases in length. In an embodiment, each R² is independently selected from a naturally or non-naturally occurring nucleobase, which, when taken together, forms a targeting sequence of 15-22 bases in length. In an embodiment, each R² is independently selected from a naturally or non-naturally occurring nucleobase, which, when taken together, forms a targeting sequence of 15-21 bases in length. In an embodiment, each R² is independently selected from a naturally or non-naturally occurring nucleobase, which, when taken together, forms a targeting sequence of 15-20 bases in length. In an embodiment, each R² is independently selected from a naturally or non-naturally occurring nucleobase, which, when taken together, forms a targeting sequence of 15-19 bases in length. In an embodiment, each R² is independently selected from a naturally or non-naturally occurring nucleobase, which, when taken together, forms a targeting sequence of 15-18 bases in length. In an embodiment, each R² is independently selected from a naturally or non-naturally occurring nucleobase, which, when taken together, forms a targeting sequence of 15-17 bases in length. In an embodiment, each R² is independently selected from a naturally or non-naturally occurring nucleobase, which, when taken together, forms a targeting sequence of 15-16 bases in length.

In another embodiment, each R² is independently selected from a naturally or non-naturally occurring nucleobase, which, when taken together, forms a targeting sequence of 16-30 bases in length. In an embodiment, each R² is independently selected from a naturally or non-naturally occurring nucleobase, which, when taken together, forms a targeting sequence of 16-29 bases in length. In an embodiment, each R² is independently selected from a naturally or non-naturally occurring nucleobase, which, when taken together, forms a targeting sequence of 16-28 bases in length. In an embodiment, each R² is independently selected from a naturally or non-naturally occurring nucleobase, which, when taken together, forms a targeting sequence of 16-27 bases in length. In an embodiment, each R² is independently selected from a naturally or non-naturally occurring nucleobase, which, when taken together, forms a targeting sequence of 16-26 bases in length. In an embodiment, each R² is independently selected from a naturally or non-naturally occurring nucleobase, which, when taken together, forms a targeting sequence of 16-25 bases in length. In an embodiment, each R² is independently selected from a naturally or non-naturally occurring nucleobase, which, when taken together, forms a targeting sequence of 16-24 bases in length. In an embodiment, each R² is independently selected from a naturally or non-naturally occurring nucleobase, which, when taken together, forms a targeting sequence of 16-23 bases in length. In an embodiment, each R² is independently selected from a naturally or non-naturally occurring nucleobase, which, when taken together, forms a targeting sequence of 16-22 bases in length. In an embodiment, each R² is independently selected from a naturally or non-naturally occurring nucleobase, which, when taken together, forms a targeting sequence of 16-21 bases in length. In an embodiment, each R² is independently selected from a naturally or non-naturally occurring nucleobase, which, when taken together, forms a targeting sequence of 16-20 bases in length. In an embodiment, each R² is independently selected from a naturally or non-naturally occurring nucleobase, which, when taken together, forms a targeting sequence of 16-19 bases in length. In an embodiment, each R² is independently selected from a naturally or non-naturally occurring nucleobase, which, when taken together, forms a targeting sequence of 16-18 bases in length. In an embodiment, each R² is independently selected from a naturally or non-naturally occurring nucleobase, which, when taken together, forms a targeting sequence of 16-17 bases in length.

In another embodiment, each R² is independently selected from a naturally or non-naturally occurring nucleobase, which, when taken together, forms a targeting sequence of 17-30 bases in length. In an embodiment, each R² is independently selected from a naturally or non-naturally occurring nucleobase, which, when taken together, forms a targeting sequence of 17-29 bases in length. In an embodiment, each R² is independently selected from a naturally or non-naturally occurring nucleobase, which, when taken together, forms a targeting sequence of 17-28 bases in length. In an embodiment, each R² is independently selected from a naturally or non-naturally occurring nucleobase, which, when taken together, forms a targeting sequence of 17-27 bases in length. In an embodiment, each R² is independently selected from a naturally or non-naturally occurring nucleobase, which, when taken together, forms a targeting sequence of 17-26 bases in length. In an embodiment, each R² is independently selected from a naturally or non-naturally occurring nucleobase, which, when taken together, forms a targeting sequence of 17-25 bases in length. In an embodiment, each R² is independently selected from a naturally or non-naturally occurring nucleobase, which, when taken together, forms a targeting sequence of 17-24 bases in length. In an embodiment, each R² is independently selected from a naturally or non-naturally occurring nucleobase, which, when taken together, forms a targeting sequence of 17-23 bases in length. In an embodiment, each R² is independently selected from a naturally or non-naturally occurring nucleobase, which, when taken together, forms a targeting sequence of 17-22 bases in length. In an embodiment, each R² is independently selected from a naturally or non-naturally occurring nucleobase, which, when taken together, forms a targeting sequence of 17-21 bases in length. In an embodiment, each R² is independently selected from a naturally or non-naturally occurring nucleobase, which, when taken together, forms a targeting sequence of 17-20 bases in length. In an embodiment, each R² is independently selected from a naturally or non-naturally occurring nucleobase, which, when taken together, forms a targeting sequence of 17-19 bases in length. In an embodiment, each R² is independently selected from a naturally or non-naturally occurring nucleobase, which, when taken together, forms a targeting sequence of 17-18 bases in

In another embodiment, each R² is independently selected from a naturally or non-naturally occurring nucleobase, which, when taken together, forms a targeting sequence of 18-30 bases in length. In an embodiment, each R² is independently selected from a naturally or non-naturally occurring nucleobase, which, when taken together, forms a targeting sequence of 18-29 bases in length. In an embodiment, each R² is independently selected from a naturally or non-naturally occurring nucleobase, which, when taken together, forms a targeting sequence of 18-28 bases in length. In an embodiment, each R² is independently selected from a naturally or non-naturally occurring nucleobase, which, when taken together, forms a targeting sequence of 18-27 bases in length. In an embodiment, each R² is independently selected from a naturally or non-naturally occurring nucleobase, which, when taken together, forms a targeting sequence of 18-26 bases in length. In an embodiment, each R² is independently selected from a naturally or non-naturally occurring nucleobase, which, when taken together, forms a targeting sequence of 18-25 bases in length. In an embodiment, each R² is independently selected from a naturally or non-naturally occurring nucleobase, which, when taken together, forms a targeting sequence of 18-24 bases in length. In an embodiment, each R² is independently selected from a naturally or non-naturally occurring nucleobase, which, when taken together, forms a targeting sequence of 18-23 bases in length. In an embodiment, each R² is independently selected from a naturally or non-naturally occurring nucleobase, which, when taken together, forms a targeting sequence of 18-22 bases in length. In an embodiment, each R² is independently selected from a naturally or non-naturally occurring nucleobase, which, when taken together, forms a targeting sequence of 18-21 bases in length. In an embodiment, each R² is independently selected from a naturally or non-naturally occurring nucleobase, which, when taken together, forms a targeting sequence of 18-20 bases in length. In an embodiment, each R² is independently selected from a naturally or non-naturally occurring nucleobase, which, when taken together, forms a targeting sequence of 18-19 bases in length.

In another embodiment, each R² is independently selected from a naturally or non-naturally occurring nucleobase, which, when taken together, forms a targeting sequence of 19-30 bases in length. In an embodiment, each R² is independently selected from a naturally or non-naturally occurring nucleobase, which, when taken together, forms a targeting sequence of 19-29 bases in length. In an embodiment, each R² is independently selected from a naturally or non-naturally occurring nucleobase, which, when taken together, forms a targeting sequence of 19-28 bases in length. In an embodiment, each R² is independently selected from a naturally or non-naturally occurring nucleobase, which, when taken together, forms a targeting sequence of 19-27 bases in length. In an embodiment, each R² is independently selected from a naturally or non-naturally occurring nucleobase, which, when taken together, forms a targeting sequence of 19-26 bases in length. In an embodiment, each R² is independently selected from a naturally or non-naturally occurring nucleobase, which, when taken together, forms a targeting sequence of 19-25 bases in length. In an embodiment, each R² is independently selected from a naturally or non-naturally occurring nucleobase, which, when taken together, forms a targeting sequence of 19-24 bases in length. In an embodiment, each R² is independently selected from a naturally or non-naturally occurring nucleobase, which, when taken together, forms a targeting sequence of 19-23 bases in length. In an embodiment, each R² is independently selected from a naturally or non-naturally occurring nucleobase, which, when taken together, forms a targeting sequence of 19-22 bases in length. In an embodiment, each R² is independently selected from a naturally or non-naturally occurring nucleobase, which, when taken together, forms a targeting sequence of 19-21 bases in length. In an embodiment, each R² is independently selected from a naturally or non-naturally occurring nucleobase, which, when taken together, forms a targeting sequence of 19-20 bases in length.

In another embodiment, each R² is independently selected from a naturally or non-naturally occurring nucleobase, which, when taken together, forms a targeting sequence of 20-30 bases in length. In an embodiment, each R² is independently selected from a naturally or non-naturally occurring nucleobase, which, when taken together, forms a targeting sequence of 20-29 bases in length. In an embodiment, each R² is independently selected from a naturally or non-naturally occurring nucleobase, which, when taken together, forms a targeting sequence of 20-28 bases in length. In an embodiment, each R² is independently selected from a naturally or non-naturally occurring nucleobase, which, when taken together, forms a targeting sequence of 20-27 bases in length. In an embodiment, each R² is independently selected from a naturally or non-naturally occurring nucleobase, which, when taken together, forms a targeting sequence of 20-26 bases in length. In an embodiment, each R² is independently selected from a naturally or non-naturally occurring nucleobase, which, when taken together, forms a targeting sequence of 20-25 bases in length. In an embodiment, each R² is independently selected from a naturally or non-naturally occurring nucleobase, which, when taken together, forms a targeting sequence of 20-24 bases in length. In an embodiment, each R² is independently selected from a naturally or non-naturally occurring nucleobase, which, when taken together, forms a targeting sequence of 20-23 bases in length. In an embodiment, each R² is independently selected from a naturally or non-naturally occurring nucleobase, which, when taken together, forms a targeting sequence of 20-22 bases in length. In an embodiment, each R² is independently selected from a naturally or non-naturally occurring nucleobase, which, when taken together, forms a targeting sequence of 20-21 bases in length.

In another embodiment, each R² is independently selected from a naturally or non-naturally occurring nucleobase, which, when taken together, forms a targeting sequence of 21-30 bases in length. In an embodiment, each R² is independently selected from a naturally or non-naturally occurring nucleobase, which, when taken together, forms a targeting sequence of 21-29 bases in length. In an embodiment, each R² is independently selected from a naturally or non-naturally occurring nucleobase, which, when taken together, forms a targeting sequence of 21-28 bases in length. In an embodiment, each R² is independently selected from a naturally or non-naturally occurring nucleobase, which, when taken together, forms a targeting sequence of 21-27 bases in length. In an embodiment, each R² is independently selected from a naturally or non-naturally occurring nucleobase, which, when taken together, forms a targeting sequence of 21-26 bases in length. In an embodiment, each R² is independently selected from a naturally or non-naturally occurring nucleobase, which, when taken together, forms a targeting sequence of 21-25 bases in length. In an embodiment, each R² is independently selected from a naturally or non-naturally occurring nucleobase, which, when taken together, forms a targeting sequence of 21-24 bases in length. In an embodiment, each R² is independently selected from a naturally or non-naturally occurring nucleobase, which, when taken together, forms a targeting sequence of 21-23 bases in length. In an embodiment, each R² is independently selected from a naturally or non-naturally occurring nucleobase, which, when taken together, forms a targeting sequence of 21-22 bases in length.

In another embodiment, each R² is independently selected from a naturally or non-naturally occurring nucleobase, which, when taken together, forms a targeting sequence of 22-30 bases in length. In an embodiment, each R² is independently selected from a naturally or non-naturally occurring nucleobase, which, when taken together, forms a targeting sequence of 22-29 bases in length. In an embodiment, each R² is independently selected from a naturally or non-naturally occurring nucleobase, which, when taken together, forms a targeting sequence of 22-28 bases in length. In an embodiment, each R² is independently selected from a naturally or non-naturally occurring nucleobase, which, when taken together, forms a targeting sequence of 22-27 bases in length. In an embodiment, each R² is independently selected from a naturally or non-naturally occurring nucleobase, which, when taken together, forms a targeting sequence of 22-26 bases in length. In an embodiment, each R² is independently selected from a naturally or non-naturally occurring nucleobase, which, when taken together, forms a targeting sequence of 22-25 bases in length. In an embodiment, each R² is independently selected from a naturally or non-naturally occurring nucleobase, which, when taken together, forms a targeting sequence of 22-24 bases in length. In an embodiment, each R² is independently selected from a naturally or non-naturally occurring nucleobase, which, when taken together, forms a targeting sequence of 22-23 bases in length.

In another embodiment, each R² is independently selected from a naturally or non-naturally occurring nucleobase, which, when taken together, forms a targeting sequence of 23-30 bases in length. In an embodiment, each R² is independently selected from a naturally or non-naturally occurring nucleobase, which, when taken together, forms a targeting sequence of 23-29 bases in length. In an embodiment, each R² is independently selected from a naturally or non-naturally occurring nucleobase, which, when taken together, forms a targeting sequence of 23-28 bases in length. In an embodiment, each R² is independently selected from a naturally or non-naturally occurring nucleobase, which, when taken together, forms a targeting sequence of 23-27 bases in length. In an embodiment, each R² is independently selected from a naturally or non-naturally occurring nucleobase, which, when taken together, forms a targeting sequence of 23-26 bases in length. In an embodiment, each R² is independently selected from a naturally or non-naturally occurring nucleobase, which, when taken together, forms a targeting sequence of 23-25 bases in length. In an embodiment, each R² is independently selected from a naturally or non-naturally occurring nucleobase, which, when taken together, forms a targeting sequence of 23-24 bases in length.

In another embodiment, each R² is independently selected from a naturally or non-naturally occurring nucleobase, which, when taken together, forms a targeting sequence of 24-30 bases in length. In an embodiment, each R² is independently selected from a naturally or non-naturally occurring nucleobase, which, when taken together, forms a targeting sequence of 24-29 bases in length. In an embodiment, each R² is independently selected from a naturally or non-naturally occurring nucleobase, which, when taken together, forms a targeting sequence of 24-28 bases in length. In an embodiment, each R² is independently selected from a naturally or non-naturally occurring nucleobase, which, when taken together, forms a targeting sequence of 24-27 bases in length. In an embodiment, each R² is independently selected from a naturally or non-naturally occurring nucleobase, which, when taken together, forms a targeting sequence of 24-26 bases in length. In an embodiment, each R² is independently selected from a naturally or non-naturally occurring nucleobase, which, when taken together, forms a targeting sequence of 24-25 bases in length.

In another embodiment, each R² is independently selected from a naturally or non-naturally occurring nucleobase, which, when taken together, forms a targeting sequence of 25-30 bases in length. In an embodiment, each R² is independently selected from a naturally or non-naturally occurring nucleobase, which, when taken together, forms a targeting sequence of 25-29 bases in length. In an embodiment, each R² is independently selected from a naturally or non-naturally occurring nucleobase, which, when taken together, forms a targeting sequence of 25-28 bases in length. In an embodiment, each R² is independently selected from a naturally or non-naturally occurring nucleobase, which, when taken together, forms a targeting sequence of 25-27 bases in length. In an embodiment, each R² is independently selected from a naturally or non-naturally occurring nucleobase, which, when taken together, forms a targeting sequence of 25-26 bases in length.

In another embodiment, each R² is independently selected from a naturally or non-naturally occurring nucleobase, which, when taken together, forms a targeting sequence of 26-30 bases in length. In an embodiment, each R² is independently selected from a naturally or non-naturally occurring nucleobase, which, when taken together, forms a targeting sequence of 26-29 bases in length. In an embodiment, each R² is independently selected from a naturally or non-naturally occurring nucleobase, which, when taken together, forms a targeting sequence of 26-28 bases in length. In an embodiment, each R² is independently selected from a naturally or non-naturally occurring nucleobase, which, when taken together, forms a targeting sequence of 26-27 bases in length.

In another embodiment, each R² is independently selected from a naturally or non-naturally occurring nucleobase, which, when taken together, forms a targeting sequence of 27-30 bases in length. In an embodiment, each R² is independently selected from a naturally or non-naturally occurring nucleobase, which, when taken together, forms a targeting sequence of 27-29 bases in length. In an embodiment, each R² is independently selected from a naturally or non-naturally occurring nucleobase, which, when taken together, forms a targeting sequence of 27-28 bases in length.

In another embodiment, each R² is independently selected from a naturally or non-naturally occurring nucleobase, which, when taken together, forms a targeting sequence of 28-30 bases in length. In an embodiment, each R² is independently selected from a naturally or non-naturally occurring nucleobase, which, when taken together, forms a targeting sequence of 28-29 bases in length. In another embodiment, each R² is independently selected from a naturally or non-naturally occurring nucleobase, which, when taken together, forms a targeting sequence of 28-30 bases in length. In an embodiment, each R² is independently selected from a naturally or non-naturally occurring nucleobase, which, when taken together, forms a targeting sequence of 29-30 bases in length.

In another embodiment, each R² is independently selected from a naturally or non-naturally occurring nucleobase, which, when taken together, forms a targeting sequence of 13 bases in length.

In another embodiment, each R² is independently selected from a naturally or non-naturally occurring nucleobase, which, when taken together, forms a targeting sequence of 14 bases in length. In another embodiment, each R² is independently selected from a naturally or non-naturally occurring nucleobase, which, when taken together, forms a targeting sequence of 15 bases in length. In another embodiment, each R² is independently selected from a naturally or non-naturally occurring nucleobase, which, when taken together, forms a targeting sequence of 16 bases in length. In another embodiment, each R² is independently selected from a naturally or non-naturally occurring nucleobase, which, when taken together, forms a targeting sequence of 17 bases in length. In another embodiment, each R² is independently selected from a naturally or non-naturally occurring nucleobase, which, when taken together, forms a targeting sequence of 18 bases in length. In another embodiment, each R² is independently selected from a naturally or non-naturally occurring nucleobase, which, when taken together, forms a targeting sequence of 19 bases in length. In another embodiment, each R² is independently selected from a naturally or non-naturally occurring nucleobase, which, when taken together, forms a targeting sequence of 20 bases in length. In another embodiment, each R² is independently selected from a naturally or non-naturally occurring nucleobase, which, when taken together, forms a targeting sequence of 21 bases in length. In another embodiment, each R² is independently selected from a naturally or non-naturally occurring nucleobase, which, when taken together, forms a targeting sequence of 22 bases in length. In another embodiment, each R² is independently selected from a naturally or non-naturally occurring nucleobase, which, when taken together, forms a targeting sequence of 23 bases in length. In another embodiment, each R² is independently selected from a naturally or non-naturally occurring nucleobase, which, when taken together, forms a targeting sequence of 24 bases in length. In another embodiment, each R² is independently selected from a naturally or non-naturally occurring nucleobase, which, when taken together, forms a targeting sequence of 25 bases in length. In another embodiment, each R² is independently selected from a naturally or non-naturally occurring nucleobase, which, when taken together, forms a targeting sequence of 26 bases in length. In another embodiment, each R² is independently selected from a naturally or non-naturally occurring nucleobase, which, when taken together, forms a targeting sequence of 27 bases in length. In another embodiment, each R² is independently selected from a naturally or non-naturally occurring nucleobase, which, when taken together, forms a targeting sequence of 28 bases in length. In another embodiment, each R² is independently selected from a naturally or non-naturally occurring nucleobase, which, when taken together, forms a targeting sequence of 29 bases in length. In another embodiment, each R² is independently selected from a naturally or non-naturally occurring nucleobase, which, when taken together, forms a targeting sequence of 30 bases in length. In certain embodiments of the antisense oligomer, or pharmaceutically acceptable salt thereof, of structural Formula (II), the salt is an HCl salt.

### Formula (III)

In some embodiments, provided herein is an antisense oligomerstructural Formula (III):

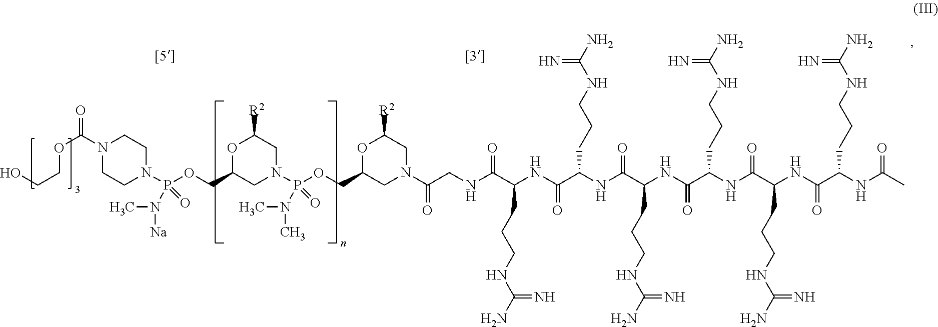

or a pharmaceutically acceptable salt thereof,
wherein:
each R² is independently selected from a naturally or non-naturally occurring nucleobase, which, when taken together, forms a targeting sequence of 13 to 30 bases, that is complementary to a target region within a pre-mRNA of the human uromodulin (UMOD) gene (SEQ ID NO: 1), wherein the target region is an intron/exon junction or an exon internal region of the human UMOD gene pre-mRNA; and
n is 11-28.

In some embodiments, n of Formula (III) is 11, 12, 13, 14, 15, 16, 17, 18, 19, 20, 21, 22, 23, 24, 25, 26, 27, or 28. In some embodiments, each R² of Formula (III) taken together forms a targeting sequence of 13, 14, 15, 16, 17, 18, 19, 20, 21, 22, 23, 24, 25, 26, 27, 28, 29, or 30 bases. In some embodiments, each R² of Formula (III) taken together forms a targeting sequence of 18-27 bases. In some embodiments, each R² of Formula (III) taken together forms a targeting sequence of 20-25 bases.

In some embodiments, n is 11, 12, 13, 14, 15, 16, 17, 18, 19, 20, 21, 22, 23, 24, 25, 26, 27, or 28. In some embodiments, each R² taken together forms a targeting sequence of 13, 14, 15, 16, 17, 18, 19, 20, 21, 22, 23, 24, 25, 26, 27, 28, 29, or 30 bases. In some embodiments, each R² taken together forms a targeting sequence of 18-27 bases. In some embodiments, each R² taken together forms a targeting sequence of 20-25 bases.

In an embodiment, n is an integer from 11 to 28. In an embodiment, n is at least 11. In an embodiment, n is up to 28. In an embodiment, n is 11-28. In an embodiment, n is 11-27. In an embodiment, n is 11-26. In an embodiment, n is 11-25. In an embodiment, n is 11-24. In an embodiment, n is 11-23. In an embodiment, n is 11-22. In an embodiment, n is 11-21. In an embodiment, n is 11-20. In an embodiment, n is 11-19. In an embodiment, n is 11-18. In an embodiment, n is 11-17. In an embodiment, n is 11-16. In an embodiment, n is 11-15. In an embodiment, n is 11-14. In an embodiment, n is 11-13. In an embodiment, n is 11-12. In an embodiment, n is 12-28. In an embodiment, n is 12-27. In an embodiment, n is 12-26. In an embodiment, n is 12-25. In an embodiment, n is 12-24. In an embodiment, n is 12-23. In an embodiment, n is 12-22. In an embodiment, n is 12-21. In an embodiment, n is 12-20. In an embodiment, n is 12-19. In an embodiment, n is 12-18. In an embodiment, n is 12-17. In an embodiment, n is 12-16. In an embodiment, n is 12-15. In an embodiment, n is 12-14. In an embodiment, n is 12-13. In an embodiment, n is 13-28. In an embodiment, n is 13-27. In an embodiment, n is 13-26. In an embodiment, n is 13-25. In an embodiment, n is 13-24. In an embodiment, n is 13-23. In an embodiment, n is 13-22. In an embodiment, n is 13-21. In an embodiment, n is 13-20. In an embodiment, n is 13-19. In an embodiment, n is 13-18. In an embodiment, n is 13-17. In an embodiment, n is 13-16. In an embodiment, n is 13-15. In an embodiment, n is 13-14. In an embodiment, n is 14-28. In an embodiment, n is 14-27. In an embodiment, n is 14-26. In an embodiment, n is 14-25. In an embodiment, n is 14-24. In an embodiment, n is 14-23. In an embodiment, n is 14-22. In an embodiment, n is 14-21. In an embodiment, n is 14-20. In an embodiment, n is 14-19. In an embodiment, n is 14-18. In an embodiment, n is 14-17. In an embodiment, n is 14-16. In an embodiment, n is 14-15. In an embodiment, n is 15-28. In an embodiment, n is 15-27. In an embodiment, n is 15-26. In an embodiment, n is 15-25. In an embodiment, n is 15-24. In an embodiment, n is 15-23. In an embodiment, n is 15-22. In an embodiment, n is 15-21. In an embodiment, n is 15-20. In an embodiment, n is 15-19. In an embodiment, n is 15-18. In an embodiment, n is 15-17. In an embodiment, n is 15-16. In an embodiment, n is 16-28. In an embodiment, n is 16-27. In an embodiment, n is 16-26. In an embodiment, n is 16-25. In an embodiment, n is 16-24. In an embodiment, n is 16-23. In an embodiment, n is 16-22. In an embodiment, n is 16-21. In an embodiment, n is 16-20. In an embodiment, n is 16-19. In an embodiment, n is 16-18. In an embodiment, n is 16-17. In an embodiment, n is 17-28. In an embodiment, n is 17-27. In an embodiment, n is 17-26. In an embodiment, n is 17-25. In an embodiment, n is 17-24. In an embodiment, n is 17-23. In an embodiment, n is 17-22. In an embodiment, n is 17-21. In an embodiment, n is 17-20. In an embodiment, n is 17-19. In an embodiment, n is 17-18. In an embodiment, n is 18-28. In an embodiment, n is 18-27. In an embodiment, n is 18-26. In an embodiment, n is 18-25. In an embodiment, n is 18-24. In an embodiment, n is 18-23. In an embodiment, n is 18-22. In an embodiment, n is 18-21. In an embodiment, n is 18-20. In an embodiment, n is 18-19. In an embodiment, n is 19-28. In an embodiment, n is 19-27. In an embodiment, n is 19-26. In an embodiment, n is 19-25. In an embodiment, n is 19-24. In an embodiment, n is 19-23. In an embodiment, n is 19-22. In an embodiment, n is 19-21. In an embodiment, n is 19-20. In an embodiment, n is 20-28. In an embodiment, n is 20-27. In an embodiment, n is 20-26. In an embodiment, n is 20-25. In an embodiment, n is 20-24. In an embodiment, n is 20-23. In an embodiment, n is 20-22. In an embodiment, n is 20-21. In an embodiment, n is 21-28. In an embodiment, n is 21-27. In an embodiment, n is 21-26. In an embodiment, n is 21-25. In an embodiment, n is 21-24. In an embodiment, n is 21-23. In an embodiment, n is 21-22. In an embodiment, n is 22-28. In an embodiment, n is 22-27. In an embodiment, n is 22-26. In an embodiment, n is 22-25. In an embodiment, n is 22-24. In an embodiment, n is 22-23. In an embodiment, n is 23-28. In an embodiment, n is 23-27. In an embodiment, n is 23-26. In an embodiment, n is 23-25. In an embodiment, n is 23-24. In an embodiment, n is 24-28. In an embodiment, n is 24-27. In an embodiment, n is 24-26. In an embodiment, n is 24-25. In an embodiment, n is 25-28. In an embodiment, n is 25-27. In an embodiment, n is 25-26. In another embodiment, n is 26-30. In an embodiment, n is 26-29. In an embodiment, n is 26-28. In an embodiment, n is 26-27. In an embodiment, n is 27-28.

In an embodiment, n is 11. In an embodiment, n is 12. In an embodiment, n is 13. In an embodiment, n is 14. In an embodiment, n is 15. In an embodiment, n is 16. In an embodiment, n is 17. In an embodiment, n is 18. In an embodiment, n is 19. In an embodiment, n is 20. In an embodiment, n is 21. In an embodiment, n is 22. In an embodiment, n is 23. In an embodiment, n is 24. In an embodiment, n is 25. In an embodiment, n is 26. In an embodiment, n is 27. In an embodiment, n is 28.

In an embodiment, each R² is independently selected from a naturally or non-naturally occurring nucleobase, which, when taken together, forms a targeting sequence of from 13 to 30 bases in length. In an embodiment, each R² is independently selected from a naturally or non-naturally occurring nucleobase, which, when taken together, forms a targeting sequence at least 13 bases in length. In an embodiment, each R² is independently selected from a naturally or non-naturally occurring nucleobase, which, when taken together, forms a targeting sequence of up to 30 bases in length.

In an embodiment, each R² is independently selected from a naturally or non-naturally occurring nucleobase, which, when taken together, forms a targeting sequence of 13-29 bases in length. In an embodiment, each R² is independently selected from a naturally or non-naturally occurring nucleobase, which, when taken together, forms a targeting sequence of 13-28 bases in length. In an embodiment, each R² is independently selected from a naturally or non-naturally occurring nucleobase, which, when taken together, forms a targeting sequence of 13-27 bases in length. In an embodiment, each R² is independently selected from a naturally or non-naturally occurring nucleobase, which, when taken together, forms a targeting sequence of 13-26 bases in length. In an embodiment, each R² is independently selected from a naturally or non-naturally occurring nucleobase, which, when taken together, forms a targeting sequence of 13-25 bases in length. In an embodiment, each R² is independently selected from a naturally or non-naturally occurring nucleobase, which, when taken together, forms a targeting sequence of 13-24 bases in length. In an embodiment, each R² is independently selected from a naturally or non-naturally occurring nucleobase, which, when taken together, forms a targeting sequence of 13-23 bases in length. In an embodiment, each R² is independently selected from a naturally or non-naturally occurring nucleobase, which, when taken together, forms a targeting sequence of 13-22 bases in length. In an embodiment, each R² is independently selected from a naturally or non-naturally occurring nucleobase, which, when taken together, forms a targeting sequence of 13-21 bases in length. In an embodiment, each R² is independently selected from a naturally or non-naturally occurring nucleobase, which, when taken together, forms a targeting sequence or 13-20 bases in length. In an embodiment, each R² is independently selected from a naturally or non-naturally occurring nucleobase, which, when taken together, forms a targeting sequence or 13-19 bases in length. In an embodiment, each R² is independently selected from a naturally or non-naturally occurring nucleobase, which, when taken together, forms a targeting sequence of 13-18 bases in length. In an embodiment, each R² is independently selected from a naturally or non-naturally occurring nucleobase, which, when taken together, forms a targeting sequence of 13-17 bases in length. In an embodiment, each R² is independently selected from a naturally or non-naturally occurring nucleobase, which, when taken together, forms a targeting sequence of 13-16 bases in length. In an embodiment, each R² is independently selected from a naturally or non-naturally occurring nucleobase, which, when taken together, forms a targeting sequence of 13-15 bases in length. In an embodiment, each R² is independently selected from a naturally or non-naturally occurring nucleobase, which, when taken together, forms a targeting sequence of 13-14 bases in length.

In another embodiment, each R² is independently selected from a naturally or non-naturally occurring nucleobase, which, when taken together, forms a targeting sequence of 14-30 bases in length. In an embodiment, each R² is independently selected from a naturally or non-naturally occurring nucleobase, which, when taken together, forms a targeting sequence of 14-29 bases in length. In an embodiment, each R² is independently selected from a naturally or non-naturally occurring nucleobase, which, when taken together, forms a targeting sequence of 14-28 bases in length. In an embodiment, each R² is independently selected from a naturally or non-naturally occurring nucleobase, which, when taken together, forms a targeting sequence of 14-27 bases in length. In an embodiment, each R² is independently selected from a naturally or non-naturally occurring nucleobase, which, when taken together, forms a targeting sequence of 14-26 bases in length. In an embodiment, each R² is independently selected from a naturally or non-naturally occurring nucleobase, which, when taken together, forms a targeting sequence of 14-25 bases in length. In an embodiment, each R² is independently selected from a naturally or non-naturally occurring nucleobase, which, when taken together, forms a targeting sequence of 14-24 bases in length. In an embodiment, each R² is independently selected from a naturally or non-naturally occurring nucleobase, which, when taken together, forms a targeting sequence of 14-23 bases in length. In an embodiment, each R² is independently selected from a naturally or non-naturally occurring nucleobase, which, when taken together, forms a targeting sequence of 14-22 bases in length. In an embodiment, each R² is independently selected from a naturally or non-naturally occurring nucleobase, which, when taken together, forms a targeting sequence of 14-21 bases in length. In an embodiment, each R² is independently selected from a naturally or non-naturally occurring nucleobase, which, when taken together, forms a targeting sequence of 14-20 bases in length. In an embodiment, each R² is independently selected from a naturally or non-naturally occurring nucleobase, which, when taken together, forms a targeting sequence of 14-19 bases in length. In an embodiment, each R² is independently selected from a naturally or non-naturally occurring nucleobase, which, when taken together, forms a targeting sequence of 14-18 bases in length. In an embodiment, each R² is independently selected from a naturally or non-naturally occurring nucleobase, which, when taken together, forms a targeting sequence of 14-17 bases in length. In an embodiment, each R² is independently selected from a naturally or non-naturally occurring nucleobase, which, when taken together, forms a targeting sequence of 14-16 bases in length. In an embodiment, each R² is independently selected from a naturally or non-naturally occurring nucleobase, which, when taken together, forms a targeting sequence of 14-15 bases in length.

In another embodiment, each R² is independently selected from a naturally or non-naturally occurring nucleobase, which, when taken together, forms a targeting sequence of 15-30 bases in length. In an embodiment, each R² is independently selected from a naturally or non-naturally occurring nucleobase, which, when taken together, forms a targeting sequence of 15-29 bases in length. In an embodiment, each R² is independently selected from a naturally or non-naturally occurring nucleobase, which, when taken together, forms a targeting sequence of 15-28 bases in length. In an embodiment, each R² is independently selected from a naturally or non-naturally occurring nucleobase, which, when taken together, forms a targeting sequence of 15-27 bases in length. In an embodiment, each R² is independently selected from a naturally or non-naturally occurring nucleobase, which, when taken together, forms a targeting sequence of 15-26 bases in length. In an embodiment, each R² is independently selected from a naturally or non-naturally occurring nucleobase, which, when taken together, forms a targeting sequence of 15-25 bases in length. In an embodiment, each R² is independently selected from a naturally or non-naturally occurring nucleobase, which, when taken together, forms a targeting sequence of 15-24 bases in length. In an embodiment, each R² is independently selected from a naturally or non-naturally occurring nucleobase, which, when taken together, forms a targeting sequence of 15-23 bases in length. In an embodiment, each R² is independently selected from a naturally or non-naturally occurring nucleobase, which, when taken together, forms a targeting sequence of 15-22 bases in length. In an embodiment, each R² is independently selected from a naturally or non-naturally occurring nucleobase, which, when taken together, forms a targeting sequence of 15-21 bases in length. In an embodiment, each R² is independently selected from a naturally or non-naturally occurring nucleobase, which, when taken together, forms a targeting sequence of 15-20 bases in length. In an embodiment, each R² is independently selected from a naturally or non-naturally occurring nucleobase, which, when taken together, forms a targeting sequence of 15-19 bases in length. In an embodiment, each R² is independently selected from a naturally or non-naturally occurring nucleobase, which, when taken together, forms a targeting sequence of 15-18 bases in length. In an embodiment, each R² is independently selected from a naturally or non-naturally occurring nucleobase, which, when taken together, forms a targeting sequence of 15-17 bases in length. In an embodiment, each R² is independently selected from a naturally or non-naturally occurring nucleobase, which, when taken together, forms a targeting sequence of 15-16 bases in length.

In another embodiment, each R² is independently selected from a naturally or non-naturally occurring nucleobase, which, when taken together, forms a targeting sequence of 16-30 bases in length. In an embodiment, each R² is independently selected from a naturally or non-naturally occurring nucleobase, which, when taken together, forms a targeting sequence of 16-29 bases in length. In an embodiment, each R² is independently selected from a naturally or non-naturally occurring nucleobase, which, when taken together, forms a targeting sequence of 16-28 bases in length. In an embodiment, each R² is independently selected from a naturally or non-naturally occurring nucleobase, which, when taken together, forms a targeting sequence of 16-27 bases in length. In an embodiment, each R² is independently selected from a naturally or non-naturally occurring nucleobase, which, when taken together, forms a targeting sequence of 16-26 bases in length. In an embodiment, each R² is independently selected from a naturally or non-naturally occurring nucleobase, which, when taken together, forms a targeting sequence of 16-25 bases in length. In an embodiment, each R² is independently selected from a naturally or non-naturally occurring nucleobase, which, when taken together, forms a targeting sequence of 16-24 bases in length. In an embodiment, each R² is independently selected from a naturally or non-naturally occurring nucleobase, which, when taken together, forms a targeting sequence of 16-23 bases in length. In an embodiment, each R² is independently selected from a naturally or non-naturally occurring nucleobase, which, when taken together, forms a targeting sequence of 16-22 bases in length. In an embodiment, each R² is independently selected from a naturally or non-naturally occurring nucleobase, which, when taken together, forms a targeting sequence of 16-21 bases in length. In an embodiment, each R² is independently selected from a naturally or non-naturally occurring nucleobase, which, when taken together, forms a targeting sequence of 16-20 bases in length. In an embodiment, each R² is independently selected from a naturally or non-naturally occurring nucleobase, which, when taken together, forms a targeting sequence of 16-19 bases in length. In an embodiment, each R² is independently selected from a naturally or non-naturally occurring nucleobase, which, when taken together, forms a targeting sequence of 16-18 bases in length. In an embodiment, each R² is independently selected from a naturally or non-naturally occurring nucleobase, which, when taken together, forms a targeting sequence of 16-17 bases in length.

In another embodiment, each R² is independently selected from a naturally or non-naturally occurring nucleobase, which, when taken together, forms a targeting sequence of 17-30 bases in length. In an embodiment, each R² is independently selected from a naturally or non-naturally occurring nucleobase, which, when taken together, forms a targeting sequence of 17-29 bases in length. In an embodiment, each R² is independently selected from a naturally or non-naturally occurring nucleobase, which, when taken together, forms a targeting sequence of 17-28 bases in length. In an embodiment, each R² is independently selected from a naturally or non-naturally occurring nucleobase, which, when taken together, forms a targeting sequence of 17-27 bases in length. In an embodiment, each R² is independently selected from a naturally or non-naturally occurring nucleobase, which, when taken together, forms a targeting sequence of 17-26 bases in length. In an embodiment, each R² is independently selected from a naturally or non-naturally occurring nucleobase, which, when taken together, forms a targeting sequence of 17-25 bases in length. In an embodiment, each R² is independently selected from a naturally or non-naturally occurring nucleobase, which, when taken together, forms a targeting sequence of 17-24 bases in length. In an embodiment, each R² is independently selected from a naturally or non-naturally occurring nucleobase, which, when taken together, forms a targeting sequence of 17-23 bases in length. In an embodiment, each R² is independently selected from a naturally or non-naturally occurring nucleobase, which, when taken together, forms a targeting sequence of 17-22 bases in length. In an embodiment, each R² is independently selected from a naturally or non-naturally occurring nucleobase, which, when taken together, forms a targeting sequence of 17-21 bases in length. In an embodiment, each R² is independently selected from a naturally or non-naturally occurring nucleobase, which, when taken together, forms a targeting sequence of 17-20 bases in length. In an embodiment, each R² is independently selected from a naturally or non-naturally occurring nucleobase, which, when taken together, forms a targeting sequence of 17-19 bases in length. In an embodiment, each R² is independently selected from a naturally or non-naturally occurring nucleobase, which, when taken together, forms a targeting sequence of 17-18 bases in length.

In another embodiment, each R² is independently selected from a naturally or non-naturally occurring nucleobase, which, when taken together, forms a targeting sequence of 18-30 bases in length. In an embodiment, each R² is independently selected from a naturally or non-naturally occurring nucleobase, which, when taken together, forms a targeting sequence of 18-29 bases in length. In an embodiment, each R² is independently selected from a naturally or non-naturally occurring nucleobase, which, when taken together, forms a targeting sequence of 18-28 bases in length. In an embodiment, each R² is independently selected from a naturally or non-naturally occurring nucleobase, which, when taken together, forms a targeting sequence of 18-27 bases in length. In an embodiment, each R² is independently selected from a naturally or non-naturally occurring nucleobase, which, when taken together, forms a targeting sequence of 18-26 bases in length. In an embodiment, each R² is independently selected from a naturally or non-naturally occurring nucleobase, which, when taken together, forms a targeting sequence of 18-25 bases in length. In an embodiment, each R² is independently selected from a naturally or non-naturally occurring nucleobase, which, when taken together, forms a targeting sequence of 18-24 bases in length. In an embodiment, each R² is independently selected from a naturally or non-naturally occurring nucleobase, which, when taken together, forms a targeting sequence of 18-23 bases in length. In an embodiment, each R² is independently selected from a naturally or non-naturally occurring nucleobase, which, when taken together, forms a targeting sequence of 18-22 bases in length. In an embodiment, each R² is independently selected from a naturally or non-naturally occurring nucleobase, which, when taken together, forms a targeting sequence of 18-21 bases in length. In an embodiment, each R² is independently selected from a naturally or non-naturally occurring nucleobase, which, when taken together, forms a targeting sequence of 18-20 bases in length. In an embodiment, each R² is independently selected from a naturally or non-naturally occurring nucleobase, which, when taken together, forms a targeting sequence of 18-19 bases in length.

In another embodiment, each R² is independently selected from a naturally or non-naturally occurring nucleobase, which, when taken together, forms a targeting sequence of 19-30 bases in length. In an embodiment, each R² is independently selected from a naturally or non-naturally occurring nucleobase, which, when taken together, forms a targeting sequence of 19-29 bases in length. In an embodiment, each R² is independently selected from a naturally or non-naturally occurring nucleobase, which, when taken together, forms a targeting sequence of 19-28 bases in length. In an embodiment, each R² is independently selected from a naturally or non-naturally occurring nucleobase, which, when taken together, forms a targeting sequence of 19-27 bases in length. In an embodiment, each R² is independently selected from a naturally or non-naturally occurring nucleobase, which, when taken together, forms a targeting sequence of 19-26 bases in length. In an embodiment, each R² is independently selected from a naturally or non-naturally occurring nucleobase, which, when taken together, forms a targeting sequence of 19-25 bases in length. In an embodiment, each R² is independently selected from a naturally or non-naturally occurring nucleobase, which, when taken together, forms a targeting sequence of 19-24 bases in length. In an embodiment, each R² is independently selected from a naturally or non-naturally occurring nucleobase, which, when taken together, forms a targeting sequence of 19-23 bases in length. In an embodiment, each R² is independently selected from a naturally or non-naturally occurring nucleobase, which, when taken together, forms a targeting sequence of 19-22 bases in length. In an embodiment, each R² is independently selected from a naturally or non-naturally occurring nucleobase, which, when taken together, forms a targeting sequence of 19-21 bases in length. In an embodiment, each R² is independently selected from a naturally or non-naturally occurring nucleobase, which, when taken together, forms a targeting sequence of 19-20 bases in length.

In another embodiment, each R² is independently selected from a naturally or non-naturally occurring nucleobase, which, when taken together, forms a targeting sequence of 20-30 bases in length. In an embodiment, each R² is independently selected from a naturally or non-naturally occurring nucleobase, which, when taken together, forms a targeting sequence of 20-29 bases in length. In an embodiment, each R² is independently selected from a naturally or non-naturally occurring nucleobase, which, when taken together, forms a targeting sequence of 20-28 bases in length. In an embodiment, each R² is independently selected from a naturally or non-naturally occurring nucleobase, which, when taken together, forms a targeting sequence of 20-27 bases in length. In an embodiment, each R² is independently selected from a naturally or non-naturally occurring nucleobase, which, when taken together, forms a targeting sequence of 20-26 bases in length. In an embodiment, each R² is independently selected from a naturally or non-naturally occurring nucleobase, which, when taken together, forms a targeting sequence of 20-25 bases in length. In an embodiment, each R² is independently selected from a naturally or non-naturally occurring nucleobase, which, when taken together, forms a targeting sequence of 20-24 bases in length. In an embodiment, each R² is independently selected from a naturally or non-naturally occurring nucleobase, which, when taken together, forms a targeting sequence of 20-23 bases in length. In an embodiment, each R² is independently selected from a naturally or non-naturally occurring nucleobase, which, when taken together, forms a targeting sequence of 20-22 bases in length. In an embodiment, each R² is independently selected from a naturally or non-naturally occurring nucleobase, which, when taken together, forms a targeting sequence of 20-21 bases in length.

In another embodiment, each R² is independently selected from a naturally or non-naturally occurring nucleobase, which, when taken together, forms a targeting sequence of 21-30 bases in length. In an embodiment, each R² is independently selected from a naturally or non-naturally occurring nucleobase, which, when taken together, forms a targeting sequence of 21-29 bases in length. In an embodiment, each R² is independently selected from a naturally or non-naturally occurring nucleobase, which, when taken together, forms a targeting sequence of 21-28 bases in length. In an embodiment, each R² is independently selected from a naturally or non-naturally occurring nucleobase, which, when taken together, forms a targeting sequence of 21-27 bases in length. In an embodiment, each R² is independently selected from a naturally or non-naturally occurring nucleobase, which, when taken together, forms a targeting sequence of 21-26 bases in length. In an embodiment, each R² is independently selected from a naturally or non-naturally occurring nucleobase, which, when taken together, forms a targeting sequence of 21-25 bases in length. In an embodiment, each R² is independently selected from a naturally or non-naturally occurring nucleobase, which, when taken together, forms a targeting sequence of 21-24 bases in length. In an embodiment, each R² is independently selected from a naturally or non-naturally occurring nucleobase, which, when taken together, forms a targeting sequence of 21-23 bases in length. In an embodiment, each R² is independently selected from a naturally or non-naturally occurring nucleobase, which, when taken together, forms a targeting sequence of 21-22 bases in length.

In another embodiment, each R² is independently selected from a naturally or non-naturally occurring nucleobase, which, when taken together, forms a targeting sequence of 22-30 bases in length. In an embodiment, each R² is independently selected from a naturally or non-naturally occurring nucleobase, which, when taken together, forms a targeting sequence of 22-29 bases in length. In an embodiment, each R² is independently selected from a naturally or non-naturally occurring nucleobase, which, when taken together, forms a targeting sequence of 22-28 bases in length. In an embodiment, each R² is independently selected from a naturally or non-naturally occurring nucleobase, which, when taken together, forms a targeting sequence of 22-27 bases in length. In an embodiment, each R² is independently selected from a naturally or non-naturally occurring nucleobase, which, when taken together, forms a targeting sequence of 22-26 bases in length. In an embodiment, each R² is independently selected from a naturally or non-naturally occurring nucleobase, which, when taken together, forms a targeting sequence of 22-25 bases in length. In an embodiment, each R² is independently selected from a naturally or non-naturally occurring nucleobase, which, when taken together, forms a targeting sequence of 22-24 bases in length. In an embodiment, each R² is independently selected from a naturally or non-naturally occurring nucleobase, which, when taken together, forms a targeting sequence of 22-23 bases in length.

In another embodiment, each R² is independently selected from a naturally or non-naturally occurring nucleobase, which, when taken together, forms a targeting sequence of 23-30 bases in length. In an embodiment, each R² is independently selected from a naturally or non-naturally occurring nucleobase, which, when taken together, forms a targeting sequence of 23-29 bases in length. In an embodiment, each R² is independently selected from a naturally or non-naturally occurring nucleobase, which, when taken together, forms a targeting sequence of 23-28 bases in length. In an embodiment, each R² is independently selected from a naturally or non-naturally occurring nucleobase, which, when taken together, forms a targeting sequence of 23-27 bases in length. In an embodiment, each R² is independently selected from a naturally or non-naturally occurring nucleobase, which, when taken together, forms a targeting sequence of 23-26 bases in length. In an embodiment, each R² is independently selected from a naturally or non-naturally occurring nucleobase, which, when taken together, forms a targeting sequence of 23-25 bases in length. In an embodiment, each R² is independently selected from a naturally or non-naturally occurring nucleobase, which, when taken together, forms a targeting sequence of 23-24 bases in length.

In another embodiment, each R² is independently selected from a naturally or non-naturally occurring nucleobase, which, when taken together, forms a targeting sequence of 24-30 bases in length. In an embodiment, each R² is independently selected from a naturally or non-naturally occurring nucleobase, which, when taken together, forms a targeting sequence of 24-29 bases in length. In an embodiment, each R² is independently selected from a naturally or non-naturally occurring nucleobase, which, when taken together, forms a targeting sequence of 24-28 bases in length. In an embodiment, each R² is independently selected from a naturally or non-naturally occurring nucleobase, which, when taken together, forms a targeting sequence of 24-27 bases in length. In an embodiment, each R² is independently selected from a naturally or non-naturally occurring nucleobase, which, when taken together, forms a targeting sequence of 24-26 bases in length. In an embodiment, each R² is independently selected from a naturally or non-naturally occurring nucleobase, which, when taken together, forms a targeting sequence of 24-25 bases in

In another embodiment, each R² is independently selected from a naturally or non-naturally occurring nucleobase, which, when taken together, forms a targeting sequence of 25-30 bases in length. In an embodiment, each R² is independently selected from a naturally or non-naturally occurring nucleobase, which, when taken together, forms a targeting sequence of 25-29 bases in length. In an embodiment, each R² is independently selected from a naturally or non-naturally occurring nucleobase, which, when taken together, forms a targeting sequence of 25-28 bases in length. In an embodiment, each R² is independently selected from a naturally or non-naturally occurring nucleobase, which, when taken together, forms a targeting sequence of 25-27 bases in length. In an embodiment, each R² is independently selected from a naturally or non-naturally occurring nucleobase, which, when taken together, forms a targeting sequence of 25-26 bases in length.

In another embodiment, each R² is independently selected from a naturally or non-naturally occurring nucleobase, which, when taken together, forms a targeting sequence of 26-30 bases in length. In an embodiment, each R² is independently selected from a naturally or non-naturally occurring nucleobase, which, when taken together, forms a targeting sequence of 26-29 bases in length. In an embodiment, each R² is independently selected from a naturally or non-naturally occurring nucleobase, which, when taken together, forms a targeting sequence of 26-28 bases in length. In an embodiment, each R² is independently selected from a naturally or non-naturally occurring nucleobase, which, when taken together, forms a targeting sequence of 26-27 bases in length.

In another embodiment, each R² is independently selected from a naturally or non-naturally occurring nucleobase, which, when taken together, forms a targeting sequence of 27-30 bases in length. In an embodiment, each R² is independently selected from a naturally or non-naturally occurring nucleobase, which, when taken together, forms a targeting sequence of 27-29 bases in length. In an embodiment, each R² is independently selected from a naturally or non-naturally occurring nucleobase, which, when taken together, forms a targeting sequence of 27-28 bases in length.

In another embodiment, each R² is independently selected from a naturally or non-naturally occurring nucleobase, which, when taken together, forms a targeting sequence of 28-30 bases in length. In an embodiment, each R² is independently selected from a naturally or non-naturally occurring nucleobase, which, when taken together, forms a targeting sequence of 28-29 bases in length. In another embodiment, each R² is independently selected from a naturally or non-naturally occurring nucleobase, which, when taken together, forms a targeting sequence of 28-30 bases in length. In an embodiment, each R² is independently selected from a naturally or non-naturally occurring nucleobase, which, when taken together, forms a targeting sequence of 29-30 bases in

In another embodiment, each R² is independently selected from a naturally or non-naturally occurring nucleobase, which, when taken together, forms a targeting sequence of 13 bases in length.

In another embodiment, each R² is independently selected from a naturally or non-naturally occurring nucleobase, which, when taken together, forms a targeting sequence of 14 bases in length. In another embodiment, each R² is independently selected from a naturally or non-naturally occurring nucleobase, which, when taken together, forms a targeting sequence of 15 bases in length. In another embodiment, each R² is independently selected from a naturally or non-naturally occurring nucleobase, which, when taken together, forms a targeting sequence of 16 bases in length. In another embodiment, each R² is independently selected from a naturally or non-naturally occurring nucleobase, which, when taken together, forms a targeting sequence of 17 bases in length. In another embodiment, each R² is independently selected from a naturally or non-naturally occurring nucleobase, which, when taken together, forms a targeting sequence of 18 bases in length. In another embodiment, each R² is independently selected from a naturally or non-naturally occurring nucleobase, which, when taken together, forms a targeting sequence of 19 bases in length. In another embodiment, each R² is independently selected from a naturally or non-naturally occurring nucleobase, which, when taken together, forms a targeting sequence of 20 bases in length. In another embodiment, each R² is independently selected from a naturally or non-naturally occurring nucleobase, which, when taken together, forms a targeting sequence of 21 bases in length. In another embodiment, each R² is independently selected from a naturally or non-naturally occurring nucleobase, which, when taken together, forms a targeting sequence of 22 bases in length. In another embodiment, each R² is independently selected from a naturally or non-naturally occurring nucleobase, which, when taken together, forms a targeting sequence of 23 bases in length. In another embodiment, each R² is independently selected from a naturally or non-naturally occurring nucleobase, which, when taken together, forms a targeting sequence of 24 bases in length. In another embodiment, each R² is independently selected from a naturally or non-naturally occurring nucleobase, which, when taken together, forms a targeting sequence of 25 bases in length. In another embodiment, each R² is independently selected from a naturally or non-naturally occurring nucleobase, which, when taken together, forms a targeting sequence of 26 bases in length. In another embodiment, each R² is independently selected from a naturally or non-naturally occurring nucleobase, which, when taken together, forms a targeting sequence of 27 bases in length. In another embodiment, each R² is independently selected from a naturally or non-naturally occurring nucleobase, which, when taken together, forms a targeting sequence of 28 bases in length. In another embodiment, each R² is independently selected from a naturally or non-naturally occurring nucleobase, which, when taken together, forms a targeting sequence of 29 bases in length. In another embodiment, each R² is independently selected from a naturally or non-naturally occurring nucleobase, which, when taken together, forms a targeting sequence of 30 bases in length.

In an embodiment, each R², taken together, forms a targeting sequence comprising SEQ ID NO: 37. In an embodiment, each R², taken together, forms a targeting sequence comprising SEQ ID NO: 32. In an embodiment, each R², taken together, forms a targeting sequence comprising SEQ ID NO: 70. In an embodiment, each R², taken together, forms a targeting sequence comprising SEQ ID NO: 74. In an embodiment, R², taken together, forms a targeting sequence comprising SEQ ID NO: 62. In an embodiment, each R², taken together, forms a targeting sequence comprising SEQ ID NO: 81. In an embodiment, each R², taken together, forms a targeting sequence comprising SEQ ID NO: 114. In an embodiment, each R², taken together, forms a targeting sequence comprising SEQ ID NO: 120.

In certain embodiments of the antisense oligomer, or pharmaceutically acceptable salt thereof, of structural Formula (III), the salt is an HCl salt.

### Formula (IV)

In some embodiments, provided herein is an antisense oligomer of Formula (III) which is an oligomer of Formula (IV):

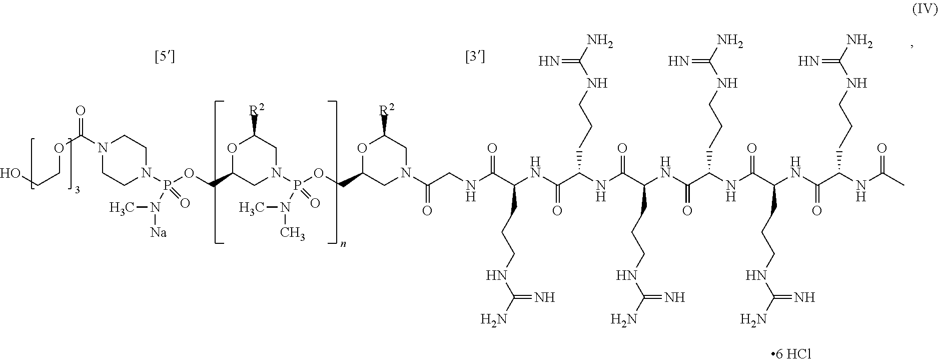

wherein:
each R² is independently selected from a naturally or non-naturally occurring nucleobase, which, when taken together, forms a targeting sequence of 13 to 30 bases, that is complementary to a target region within a pre-mRNA of the human uromodulin (UMOD) gene (SEQ ID NO: 1), wherein the target region is an intron/exon junction or an exon internal region of the human UMOD gene pre-mRNA; and n is 11-28.

In some embodiments, n is 11, 12, 13, 14, 15, 16, 17, 18, 19, 20, 21, 22, 23, 24, 25, 26, 27, or 28. In some embodiments, each R² taken together forms a targeting sequence of 13, 14, 15, 16, 17, 18, 19, 20, 21, 22, 23, 24, 25, 26, 27, 28, 29, or 30 bases. In some embodiments, each R² taken together forms a targeting sequence of 18-27 bases. In some embodiments, each R² taken together forms a targeting sequence of 20-25 bases.

In an embodiment, n is an integer from 11 to 28. In an embodiment, n is at least 11. In an embodiment, n is up to 28. In an embodiment, n is 11-28. In an embodiment, n is 11-27. In an embodiment, n is 11-26. In an embodiment, n is 11-25. In an embodiment, n is 11-24. In an embodiment, n is 11-23. In an embodiment, n is 11-22. In an embodiment, n is 11-21. In an embodiment, n is 11-20. In an embodiment, n is 11-19. In an embodiment, n is 11-18. In an embodiment, n is 11-17. In an embodiment, n is 11-16. In an embodiment, n is 11-15. In an embodiment, n is 11-14. In an embodiment, n is 11-13. In an embodiment, n is 11-12. In an embodiment, n is 12-28. In an embodiment, n is 12-27. In an embodiment, n is 12-26. In an embodiment, n is 12-25. In an embodiment, n is 12-24. In an embodiment, n is 12-23. In an embodiment, n is 12-22. In an embodiment, n is 12-21. In an embodiment, n is 12-20. In an embodiment, n is 12-19. In an embodiment, n is 12-18. In an embodiment, n is 12-17. In an embodiment, n is 12-16. In an embodiment, n is 12-15. In an embodiment, n is 12-14. In an embodiment, n is 12-13. In an embodiment, n is 13-28. In an embodiment, n is 13-27. In an embodiment, n is 13-26. In an embodiment, n is 13-25. In an embodiment, n is 13-24. In an embodiment, n is 13-23. In an embodiment, n is 13-22. In an embodiment, n is 13-21. In an embodiment, n is 13-20. In an embodiment, n is 13-19. In an embodiment, n is 13-18. In an embodiment, n is 13-17. In an embodiment, n is 13-16. In an embodiment, n is 13-15. In an embodiment, n is 13-14. In an embodiment, n is 14-28. In an embodiment, n is 14-27. In an embodiment, n is 14-26. In an embodiment, n is 14-25. In an embodiment, n is 14-24. In an embodiment, n is 14-23. In an embodiment, n is 14-22. In an embodiment, n is 14-21. In an embodiment, n is 14-20. In an embodiment, n is 14-19. In an embodiment, n is 14-18. In an embodiment, n is 14-17. In an embodiment, n is 14-16. In an embodiment, n is 14-15. In an embodiment, n is 15-28. In an embodiment, n is 15-27. In an embodiment, n is 15-26. In an embodiment, n is 15-25. In an embodiment, n is 15-24. In an embodiment, n is 15-23. In an embodiment, n is 15-22. In an embodiment, n is 15-21. In an embodiment, n is 15-20. In an embodiment, n is 15-19. In an embodiment, n is 15-18. In an embodiment, n is 15-17. In an embodiment, n is 15-16. In an embodiment, n is 16-28. In an embodiment, n is 16-27. In an embodiment, n is 16-26. In an embodiment, n is 16-25. In an embodiment, n is 16-24. In an embodiment, n is 16-23. In an embodiment, n is 16-22. In an embodiment, n is 16-21. In an embodiment, n is 16-20. In an embodiment, n is 16-19. In an embodiment, n is 16-18. In an embodiment, n is 16-17. In an embodiment, n is 17-28. In an embodiment, n is 17-27. In an embodiment, n is 17-26. In an embodiment, n is 17-25. In an embodiment, n is 17-24. In an embodiment, n is 17-23. In an embodiment, n is 17-22. In an embodiment, n is 17-21. In an embodiment, n is 17-20. In an embodiment, n is 17-19. In an embodiment, n is 17-18. In an embodiment, n is 18-28. In an embodiment, n is 18-27. In an embodiment, n is 18-26. In an embodiment, n is 18-25. In an embodiment, n is 18-24. In an embodiment, n is 18-23. In an embodiment, n is 18-22. In an embodiment, n is 18-21. In an embodiment, n is 18-20. In an embodiment, n is 18-19. In an embodiment, n is 19-28. In an embodiment, n is 19-27. In an embodiment, n is 19-26. In an embodiment, n is 19-25. In an embodiment, n is 19-24. In an embodiment, n is 19-23. In an embodiment, n is 19-22. In an embodiment, n is 19-21. In an embodiment, n is 19-20. In an embodiment, n is 20-28. In an embodiment, n is 20-27. In an embodiment, n is 20-26. In an embodiment, n is 20-25. In an embodiment, n is 20-24. In an embodiment, n is 20-23. In an embodiment, n is 20-22. In an embodiment, n is 20-21. In an embodiment, n is 21-28. In an embodiment, n is 21-27. In an embodiment, n is 21-26. In an embodiment, n is 21-25. In an embodiment, n is 21-24. In an embodiment, n is 21-23. In an embodiment, n is 21-22. In an embodiment, n is 22-28. In an embodiment, n is 22-27. In an embodiment, n is 22-26. In an embodiment, n is 22-25. In an embodiment, n is 22-24. In an embodiment, n is 22-23. In an embodiment, n is 23-28. In an embodiment, n is 23-27. In an embodiment, n is 23-26. In an embodiment, n is 23-25. In an embodiment, n is 23-24. In an embodiment, n is 24-28. In an embodiment, n is 24-27. In an embodiment, n is 24-26. In an embodiment, n is 24-25. In an embodiment, n is 25-28. In an embodiment, n is 25-27. In an embodiment, n is 25-26. In another embodiment, n is 26-30. In an embodiment, n is 26-29. In an embodiment, n is 26-28. In an embodiment, n is 26-27. In an embodiment, n is 27-28.

In an embodiment, n is 11. In an embodiment, n is 12. In an embodiment, n is 13. In an embodiment, n is 14. In an embodiment, n is 15. In an embodiment, n is 16. In an embodiment, n is 17. In an embodiment, n is 18. In an embodiment, n is 19. In an embodiment, n is 20. In an embodiment, n is 21. In an embodiment, n is 22. In an embodiment, n is 23. In an embodiment, n is 24. In an embodiment, n is 25. In an embodiment, n is 26. In an embodiment, n is 27. In an embodiment, n is 28.

In an embodiment, each R² is independently selected from a naturally or non-naturally occurring nucleobase, which, when taken together, forms a targeting sequence of from 13 to 30 bases in length. In an embodiment, each R² is independently selected from a naturally or non-naturally occurring nucleobase, which, when taken together, forms a targeting sequence at least 13 bases in length. In an embodiment, each R² is independently selected from a naturally or non-naturally occurring nucleobase, which, when taken together, forms a targeting sequence of up to 30 bases in length.

In an embodiment, each R² is independently selected from a naturally or non-naturally occurring nucleobase, which, when taken together, forms a targeting sequence 13-29 bases in length. In an embodiment, each R² is independently selected from a naturally or non-naturally occurring nucleobase, which, when taken together, forms a targeting sequence 13-28 bases in length. In an embodiment, each R² is independently selected from a naturally or non-naturally occurring nucleobase, which, when taken together, forms a targeting sequence 13-27 bases in length. In an embodiment, each R² is independently selected from a naturally or non-naturally occurring nucleobase, which, when taken together, forms a targeting sequence 13-26 bases in length. In an embodiment, each R² is independently selected from a naturally or non-naturally occurring nucleobase, which, when taken together, forms a targeting sequence 13-25 bases in length. In an embodiment, each R² is independently selected from a naturally or non-naturally occurring nucleobase, which, when taken together, forms a targeting sequence 13-24 bases in length. In an embodiment, each R² is independently selected from a naturally or non-naturally occurring nucleobase, which, when taken together, forms a targeting sequence 13-23 bases in length. In an embodiment, each R² is independently selected from a naturally or non-naturally occurring nucleobase, which, when taken together, forms a targeting sequence 13-22 bases in length. In an embodiment, each R² is independently selected from a naturally or non-naturally occurring nucleobase, which, when taken together, forms a targeting sequence 13-21 bases in length. In an embodiment, each R² is independently selected from a naturally or non-naturally occurring nucleobase, which, when taken together, forms a targeting sequence 13-20 bases in length. In an embodiment, each R² is independently selected from a naturally or non-naturally occurring nucleobase, which, when taken together, forms a targeting sequence 13-19 bases in length. In an embodiment, each R² is independently selected from a naturally or non-naturally occurring nucleobase, which, when taken together, forms a targeting sequence 13-18 bases in length. In an embodiment, each R² is independently selected from a naturally or non-naturally occurring nucleobase, which, when taken together, forms a targeting sequence 13-17 bases in length. In an embodiment, each R² is independently selected from a naturally or non-naturally occurring nucleobase, which, when taken together, forms a targeting sequence 13-16 bases in length. In an embodiment, each R² is independently selected from a naturally or non-naturally occurring nucleobase, which, when taken together, forms a targeting sequence 13-15 bases in length. In an embodiment, each R² is independently selected from a naturally or non-naturally occurring nucleobase, which, when taken together, forms a targeting sequence 13-14 bases in length.

In another embodiment, each R² is independently selected from a naturally or non-naturally occurring nucleobase, which, when taken together, forms a targeting sequence 14-30 bases in length. In an embodiment, each R² is independently selected from a naturally or non-naturally occurring nucleobase, which, when taken together, forms a targeting sequence 14-29 bases in length. In an embodiment, each R² is independently selected from a naturally or non-naturally occurring nucleobase, which, when taken together, forms a targeting sequence 14-28 bases in length. In an embodiment, each R² is independently selected from a naturally or non-naturally occurring nucleobase, which, when taken together, forms a targeting sequence 14-27 bases in length. In an embodiment, each R² is independently selected from a naturally or non-naturally occurring nucleobase, which, when taken together, forms a targeting sequence 14-26 bases in length. In an embodiment, each R² is independently selected from a naturally or non-naturally occurring nucleobase, which, when taken together, forms a targeting sequence 14-25 bases in length. In an embodiment, each R² is independently selected from a naturally or non-naturally occurring nucleobase, which, when taken together, forms a targeting sequence 14-24 bases in length. In an embodiment, each R² is independently selected from a naturally or non-naturally occurring nucleobase, which, when taken together, forms a targeting sequence 14-23 bases in length. In an embodiment, each R² is independently selected from a naturally or non-naturally occurring nucleobase, which, when taken together, forms a targeting sequence 14-22 bases in length. In an embodiment, each R² is independently selected from a naturally or non-naturally occurring nucleobase, which, when taken together, forms a targeting sequence 14-21 bases in length. In an embodiment, each R² is independently selected from a naturally or non-naturally occurring nucleobase, which, when taken together, forms a targeting sequence 14-20 bases in length. In an embodiment, each R² is independently selected from a naturally or non-naturally occurring nucleobase, which, when taken together, forms a targeting sequence 14-19 bases in length. In an embodiment, each R² is independently selected from a naturally or non-naturally occurring nucleobase, which, when taken together, forms a targeting sequence 14-18 bases in length. In an embodiment, each R² is independently selected from a naturally or non-naturally occurring nucleobase, which, when taken together, forms a targeting sequence 14-17 bases in length. In an embodiment, each R² is independently selected from a naturally or non-naturally occurring nucleobase, which, when taken together, forms a targeting sequence 14-16 bases in length. In an embodiment, each R² is independently selected from a naturally or non-naturally occurring nucleobase, which, when taken together, forms a targeting sequence 14-15 bases in length.

In another embodiment, each R² is independently selected from a naturally or non-naturally occurring nucleobase, which, when taken together, forms a targeting sequence 15-30 bases in length. In an embodiment, each R² is independently selected from a naturally or non-naturally occurring nucleobase, which, when taken together, forms a targeting sequence 15-29 bases in length. In an embodiment, each R² is independently selected from a naturally or non-naturally occurring nucleobase, which, when taken together, forms a targeting sequence 15-28 bases in length. In an embodiment, each R² is independently selected from a naturally or non-naturally occurring nucleobase, which, when taken together, forms a targeting sequence 15-27 bases in length. In an embodiment, each R² is independently selected from a naturally or non-naturally occurring nucleobase, which, when taken together, forms a targeting sequence 15-26 bases in length. In an embodiment, each R² is independently selected from a naturally or non-naturally occurring nucleobase, which, when taken together, forms a targeting sequence 15-25 bases in length. In an embodiment, each R² is independently selected from a naturally or non-naturally occurring nucleobase, which, when taken together, forms a targeting sequence 15-24 bases in length. In an embodiment, each R² is independently selected from a naturally or non-naturally occurring nucleobase, which, when taken together, forms a targeting sequence 15-23 bases in length. In an embodiment, each R² is independently selected from a naturally or non-naturally occurring nucleobase, which, when taken together, forms a targeting sequence 15-22 bases in length. In an embodiment, each R² is independently selected from a naturally or non-naturally occurring nucleobase, which, when taken together, forms a targeting sequence 15-21 bases in length. In an embodiment, each R² is independently selected from a naturally or non-naturally occurring nucleobase, which, when taken together, forms a targeting sequence 15-20 bases in length. In an embodiment, each R² is independently selected from a naturally or non-naturally occurring nucleobase, which, when taken together, forms a targeting sequence 15-19 bases in length. In an embodiment, each R² is independently selected from a naturally or non-naturally occurring nucleobase, which, when taken together, forms a targeting sequence 15-18 bases in length. In an embodiment, each R² is independently selected from a naturally or non-naturally occurring nucleobase, which, when taken together, forms a targeting sequence 15-17 bases in length. In an embodiment, each R² is independently selected from a naturally or non-naturally occurring nucleobase, which, when taken together, forms a targeting sequence 15-16 bases in length.

In another embodiment, each R² is independently selected from a naturally or non-naturally occurring nucleobase, which, when taken together, forms a targeting sequence 16-30 bases in length. In an embodiment, each R² is independently selected from a naturally or non-naturally occurring nucleobase, which, when taken together, forms a targeting sequence 16-29 bases in length. In an embodiment, each R² is independently selected from a naturally or non-naturally occurring nucleobase, which, when taken together, forms a targeting sequence 16-28 bases in length. In an embodiment, each R² is independently selected from a naturally or non-naturally occurring nucleobase, which, when taken together, forms a targeting sequence 16-27 bases in length. In an embodiment, each R² is independently selected from a naturally or non-naturally occurring nucleobase, which, when taken together, forms a targeting sequence 16-26 bases in length. In an embodiment, each R² is independently selected from a naturally or non-naturally occurring nucleobase, which, when taken together, forms a targeting sequence 16-25 bases in length. In an embodiment, each R² is independently selected from a naturally or non-naturally occurring nucleobase, which, when taken together, forms a targeting sequence 16-24 bases in length. In an embodiment, each R² is independently selected from a naturally or non-naturally occurring nucleobase, which, when taken together, forms a targeting sequence 16-23 bases in length. In an embodiment, each R² is independently selected from a naturally or non-naturally occurring nucleobase, which, when taken together, forms a targeting sequence 16-22 bases in length. In an embodiment, each R² is independently selected from a naturally or non-naturally occurring nucleobase, which, when taken together, forms a targeting sequence 16-21 bases in length. In an embodiment, each R² is independently selected from a naturally or non-naturally occurring nucleobase, which, when taken together, forms a targeting sequence 16-20 bases in length. In an embodiment, each R² is independently selected from a naturally or non-naturally occurring nucleobase, which, when taken together, forms a targeting sequence 16-19 bases in length. In an embodiment, each R² is independently selected from a naturally or non-naturally occurring nucleobase, which, when taken together, forms a targeting sequence 16-18 bases in length. In an embodiment, each R² is independently selected from a naturally or non-naturally occurring nucleobase, which, when taken together, forms a targeting sequence 16-17 bases in length.

In another embodiment, each R² is independently selected from a naturally or non-naturally occurring nucleobase, which, when taken together, forms a targeting sequence 17-30 bases in length. In an embodiment, each R² is independently selected from a naturally or non-naturally occurring nucleobase, which, when taken together, forms a targeting sequence 17-29 bases in length. In an embodiment, each R² is independently selected from a naturally or non-naturally occurring nucleobase, which, when taken together, forms a targeting sequence 17-28 bases in length. In an embodiment, each R² is independently selected from a naturally or non-naturally occurring nucleobase, which, when taken together, forms a targeting sequence 17-27 bases in length. In an embodiment, each R² is independently selected from a naturally or non-naturally occurring nucleobase, which, when taken together, forms a targeting sequence 17-26 bases in length. In an embodiment, each R² is independently selected from a naturally or non-naturally occurring nucleobase, which, when taken together, forms a targeting sequence 17-25 bases in length. In an embodiment, each R² is independently selected from a naturally or non-naturally occurring nucleobase, which, when taken together, forms a targeting sequence 17-24 bases in length. In an embodiment, each R² is independently selected from a naturally or non-naturally occurring nucleobase, which, when taken together, forms a targeting sequence 17-23 bases in length. In an embodiment, each R² is independently selected from a naturally or non-naturally occurring nucleobase, which, when taken together, forms a targeting sequence 17-22 bases in length. In an embodiment, each R² is independently selected from a naturally or non-naturally occurring nucleobase, which, when taken together, forms a targeting sequence 17-21 bases in length. In an embodiment, each R² is independently selected from a naturally or non-naturally occurring nucleobase, which, when taken together, forms a targeting sequence 17-20 bases in length. In an embodiment, each R² is independently selected from a naturally or non-naturally occurring nucleobase, which, when taken together, forms a targeting sequence 17-19 bases in length. In an embodiment, each R² is independently selected from a naturally or non-naturally occurring nucleobase, which, when taken together, forms a targeting sequence 17-18 bases in

In another embodiment, each R² is independently selected from a naturally or non-naturally occurring nucleobase, which, when taken together, forms a targeting sequence 18-30 bases in length. In an embodiment, each R² is independently selected from a naturally or non-naturally occurring nucleobase, which, when taken together, forms a targeting sequence 18-29 bases in length. In an embodiment, each R² is independently selected from a naturally or non-naturally occurring nucleobase, which, when taken together, forms a targeting sequence 18-28 bases in length. In an embodiment, each R² is independently selected from a naturally or non-naturally occurring nucleobase, which, when taken together, forms a targeting sequence 18-27 bases in length. In an embodiment, each R² is independently selected from a naturally or non-naturally occurring nucleobase, which, when taken together, forms a targeting sequence 18-26 bases in length. In an embodiment, each R² is independently selected from a naturally or non-naturally occurring nucleobase, which, when taken together, forms a targeting sequence 18-25 bases in length. In an embodiment, each R² is independently selected from a naturally or non-naturally occurring nucleobase, which, when taken together, forms a targeting sequence 18-24 bases in length. In an embodiment, each R² is independently selected from a naturally or non-naturally occurring nucleobase, which, when taken together, forms a targeting sequence 18-23 bases in length. In an embodiment, each R² is independently selected from a naturally or non-naturally occurring nucleobase, which, when taken together, forms a targeting sequence 18-22 bases in length. In an embodiment, each R² is independently selected from a naturally or non-naturally occurring nucleobase, which, when taken together, forms a targeting sequence 18-21 bases in length. In an embodiment, each R² is independently selected from a naturally or non-naturally occurring nucleobase, which, when taken together, forms a targeting sequence 18-20 bases in length. In an embodiment, each R² is independently selected from a naturally or non-naturally occurring nucleobase, which, when taken together, forms a targeting sequence 18-19 bases in length.

In another embodiment, each R² is independently selected from a naturally or non-naturally occurring nucleobase, which, when taken together, forms a targeting sequence 19-30 bases in length. In an embodiment, each R² is independently selected from a naturally or non-naturally occurring nucleobase, which, when taken together, forms a targeting sequence 19-29 bases in length. In an embodiment, each R² is independently selected from a naturally or non-naturally occurring nucleobase, which, when taken together, forms a targeting sequence 19-28 bases in length. In an embodiment, each R² is independently selected from a naturally or non-naturally occurring nucleobase, which, when taken together, forms a targeting sequence 19-27 bases in length. In an embodiment, each R² is independently selected from a naturally or non-naturally occurring nucleobase, which, when taken together, forms a targeting sequence 19-26 bases in length. In an embodiment, each R² is independently selected from a naturally or non-naturally occurring nucleobase, which, when taken together, forms a targeting sequence 19-25 bases in length. In an embodiment, each R² is independently selected from a naturally or non-naturally occurring nucleobase, which, when taken together, forms a targeting sequence 19-24 bases in length. In an embodiment, each R² is independently selected from a naturally or non-naturally occurring nucleobase, which, when taken together, forms a targeting sequence 19-23 bases in length. In an embodiment, each R² is independently selected from a naturally or non-naturally occurring nucleobase, which, when taken together, forms a targeting sequence 19-22 bases in length. In an embodiment, each R² is independently selected from a naturally or non-naturally occurring nucleobase, which, when taken together, forms a targeting sequence 19-21 bases in length. In an embodiment, each R² is independently selected from a naturally or non-naturally occurring nucleobase, which, when taken together, forms a targeting sequence 19-20 bases in length.

In another embodiment, each R² is independently selected from a naturally or non-naturally occurring nucleobase, which, when taken together, forms a targeting sequence 20-30 bases in length. In an embodiment, each R² is independently selected from a naturally or non-naturally occurring nucleobase, which, when taken together, forms a targeting sequence 20-29 bases in length. In an embodiment, each R² is independently selected from a naturally or non-naturally occurring nucleobase, which, when taken together, forms a targeting sequence 20-28 bases in length. In an embodiment, each R² is independently selected from a naturally or non-naturally occurring nucleobase, which, when taken together, forms a targeting sequence 20-27 bases in length. In an embodiment, each R² is independently selected from a naturally or non-naturally occurring nucleobase, which, when taken together, forms a targeting sequence 20-26 bases in length. In an embodiment, each R² is independently selected from a naturally or non-naturally occurring nucleobase, which, when taken together, forms a targeting sequence 20-25 bases in length. In an embodiment, each R² is independently selected from a naturally or non-naturally occurring nucleobase, which, when taken together, forms a targeting sequence 20-24 bases in length. In an embodiment, each R² is independently selected from a naturally or non-naturally occurring nucleobase, which, when taken together, forms a targeting sequence 20-23 bases in length. In an embodiment, each R² is independently selected from a naturally or non-naturally occurring nucleobase, which, when taken together, forms a targeting sequence 20-22 bases in length. In an embodiment, each R² is independently selected from a naturally or non-naturally occurring nucleobase, which, when taken together, forms a targeting sequence 20-21 bases in length.

In another embodiment, each R² is independently selected from a naturally or non-naturally occurring nucleobase, which, when taken together, forms a targeting sequence 21-30 bases in length. In an embodiment, each R² is independently selected from a naturally or non-naturally occurring nucleobase, which, when taken together, forms a targeting sequence 21-29 bases in length. In an embodiment, each R² is independently selected from a naturally or non-naturally occurring nucleobase, which, when taken together, forms a targeting sequence 21-28 bases in length. In an embodiment, each R² is independently selected from a naturally or non-naturally occurring nucleobase, which, when taken together, forms a targeting sequence 21-27 bases in length. In an embodiment, each R² is independently selected from a naturally or non-naturally occurring nucleobase, which, when taken together, forms a targeting sequence 21-26 bases in length. In an embodiment, each R² is independently selected from a naturally or non-naturally occurring nucleobase, which, when taken together, forms a targeting sequence 21-25 bases in length. In an embodiment, each R² is independently selected from a naturally or non-naturally occurring nucleobase, which, when taken together, forms a targeting sequence 21-24 bases in length. In an embodiment, each R² is independently selected from a naturally or non-naturally occurring nucleobase, which, when taken together, forms a targeting sequence 21-23 bases in length. In an embodiment, each R² is independently selected from a naturally or non-naturally occurring nucleobase, which, when taken together, forms a targeting sequence 21-22 bases in length.

In another embodiment, each R² is independently selected from a naturally or non-naturally occurring nucleobase, which, when taken together, forms a targeting sequence 22-30 bases in length. In an embodiment, each R² is independently selected from a naturally or non-naturally occurring nucleobase, which, when taken together, forms a targeting sequence 22-29 bases in length. In an embodiment, each R² is independently selected from a naturally or non-naturally occurring nucleobase, which, when taken together, forms a targeting sequence 22-28 bases in length. In an embodiment, each R² is independently selected from a naturally or non-naturally occurring nucleobase, which, when taken together, forms a targeting sequence 22-27 bases in length. In an embodiment, each R² is independently selected from a naturally or non-naturally occurring nucleobase, which, when taken together, forms a targeting sequence 22-26 bases in length. In an embodiment, each R² is independently selected from a naturally or non-naturally occurring nucleobase, which, when taken together, forms a targeting sequence 22-25 bases in length. In an embodiment, each R² is independently selected from a naturally or non-naturally occurring nucleobase, which, when taken together, forms a targeting sequence 22-24 bases in length. In an embodiment, each R² is independently selected from a naturally or non-naturally occurring nucleobase, which, when taken together, forms a targeting sequence 22-23 bases in length.

In another embodiment, each R² is independently selected from a naturally or non-naturally occurring nucleobase, which, when taken together, forms a targeting sequence 23-30 bases in length. In an embodiment, each R² is independently selected from a naturally or non-naturally occurring nucleobase, which, when taken together, forms a targeting sequence 23-29 bases in length. In an embodiment, each R² is independently selected from a naturally or non-naturally occurring nucleobase, which, when taken together, forms a targeting sequence 23-28 bases in length. In an embodiment, each R² is independently selected from a naturally or non-naturally occurring nucleobase, which, when taken together, forms a targeting sequence 23-27 bases in length. In an embodiment, each R² is independently selected from a naturally or non-naturally occurring nucleobase, which, when taken together, forms a targeting sequence 23-26 bases in length. In an embodiment, each R² is independently selected from a naturally or non-naturally occurring nucleobase, which, when taken together, forms a targeting sequence 23-25 bases in length. In an embodiment, each R² is independently selected from a naturally or non-naturally occurring nucleobase, which, when taken together, forms a targeting sequence 23-24 bases in length.

In another embodiment, each R² is independently selected from a naturally or non-naturally occurring nucleobase, which, when taken together, forms a targeting sequence 24-30 bases in length. In an embodiment, each R² is independently selected from a naturally or non-naturally occurring nucleobase, which, when taken together, forms a targeting sequence 24-29 bases in length. In an embodiment, each R² is independently selected from a naturally or non-naturally occurring nucleobase, which, when taken together, forms a targeting sequence 24-28 bases in length. In an embodiment, each R² is independently selected from a naturally or non-naturally occurring nucleobase, which, when taken together, forms a targeting sequence 24-27 bases in length. In an embodiment, each R² is independently selected from a naturally or non-naturally occurring nucleobase, which, when taken together, forms a targeting sequence 24-26 bases in length. In an embodiment, each R² is independently selected from a naturally or non-naturally occurring nucleobase, which, when taken together, forms a targeting sequence 24-25 bases in length.

In another embodiment, each R² is independently selected from a naturally or non-naturally occurring nucleobase, which, when taken together, forms a targeting sequence 25-30 bases in length. In an embodiment, each R² is independently selected from a naturally or non-naturally occurring nucleobase, which, when taken together, forms a targeting sequence 25-29 bases in length. In an embodiment, each R² is independently selected from a naturally or non-naturally occurring nucleobase, which, when taken together, forms a targeting sequence 25-28 bases in length. In an embodiment, each R² is independently selected from a naturally or non-naturally occurring nucleobase, which, when taken together, forms a targeting sequence 25-27 bases in length. In an embodiment, each R² is independently selected from a naturally or non-naturally occurring nucleobase, which, when taken together, forms a targeting sequence 25-26 bases in length.

In another embodiment, each R² is independently selected from a naturally or non-naturally occurring nucleobase, which, when taken together, forms a targeting sequence 26-30 bases in length. In an embodiment, each R² is independently selected from a naturally or non-naturally occurring nucleobase, which, when taken together, forms a targeting sequence 26-29 bases in length. In an embodiment, each R² is independently selected from a naturally or non-naturally occurring nucleobase, which, when taken together, forms a targeting sequence 26-28 bases in length. In an embodiment, each R² is independently selected from a naturally or non-naturally occurring nucleobase, which, when taken together, forms a targeting sequence 26-27 bases in length.

In another embodiment, each R² is independently selected from a naturally or non-naturally occurring nucleobase, which, when taken together, forms a targeting sequence 27-30 bases in length. In an embodiment, each R² is independently selected from a naturally or non-naturally occurring nucleobase, which, when taken together, forms a targeting sequence 27-29 bases in length. In an embodiment, each R² is independently selected from a naturally or non-naturally occurring nucleobase, which, when taken together, forms a targeting sequence 27-28 bases in length.

In another embodiment, each R² is independently selected from a naturally or non-naturally occurring nucleobase, which, when taken together, forms a targeting sequence 28-30 bases in length. In an embodiment, each R² is independently selected from a naturally or non-naturally occurring nucleobase, which, when taken together, forms a targeting sequence 28-29 bases in length. In another embodiment, each R² is independently selected from a naturally or non-naturally occurring nucleobase, which, when taken together, forms a targeting sequence 28-30 bases in length. In an embodiment, each R² is independently selected from a naturally or non-naturally occurring nucleobase, which, when taken together, forms a targeting sequence 29-30 bases in length.

In another embodiment, each R² is independently selected from a naturally or non-naturally occurring nucleobase, which, when taken together, forms a targeting sequence 13 bases in length.

In another embodiment, each R² is independently selected from a naturally or non-naturally occurring nucleobase, which, when taken together, forms a targeting sequence 14 bases in length. In another embodiment, each R² is independently selected from a naturally or non-naturally occurring nucleobase, which, when taken together, forms a targeting sequence 15 bases in length. In another embodiment, each R² is independently selected from a naturally or non-naturally occurring nucleobase, which, when taken together, forms a targeting sequence 16 bases in length. In another embodiment, each R² is independently selected from a naturally or non-naturally occurring nucleobase, which, when taken together, forms a targeting sequence 17 bases in length. In another embodiment, each R² is independently selected from a naturally or non-naturally occurring nucleobase, which, when taken together, forms a targeting sequence 18 bases in length. In another embodiment, each R² is independently selected from a naturally or non-naturally occurring nucleobase, which, when taken together, forms a targeting sequence 19 bases in length. In another embodiment, each R² is independently selected from a naturally or non-naturally occurring nucleobase, which, when taken together, forms a targeting sequence 20 bases in length. In another embodiment, each R² is independently selected from a naturally or non-naturally occurring nucleobase, which, when taken together, forms a targeting sequence 21 bases in length. In another embodiment, each R² is independently selected from a naturally or non-naturally occurring nucleobase, which, when taken together, forms a targeting sequence 22 bases in length. In another embodiment, each R² is independently selected from a naturally or non-naturally occurring nucleobase, which, when taken together, forms a targeting sequence 23 bases in length. In another embodiment, each R² is independently selected from a naturally or non-naturally occurring nucleobase, which, when taken together, forms a targeting sequence 24 bases in length. In another embodiment, each R² is independently selected from a naturally or non-naturally occurring nucleobase, which, when taken together, forms a targeting sequence 25 bases in length. In another embodiment, each R² is independently selected from a naturally or non-naturally occurring nucleobase, which, when taken together, forms a targeting sequence 26 bases in length. In another embodiment, each R² is independently selected from a naturally or non-naturally occurring nucleobase, which, when taken together, forms a targeting sequence 27 bases in length. In another embodiment, each R² is independently selected from a naturally or non-naturally occurring nucleobase, which, when taken together, forms a targeting sequence 28 bases in length. In another embodiment, each R² is independently selected from a naturally or non-naturally occurring nucleobase, which, when taken together, forms a targeting sequence 29 bases in length. In another embodiment, each R² is independently selected from a naturally or non-naturally occurring nucleobase, which, when taken together, forms a targeting sequence 30 bases in length.

In an embodiment, each R², taken together, forms a targeting sequence comprising SEQ ID NO: 37. In an embodiment, each R², taken together, forms a targeting sequence comprising SEQ ID NO: 32. In an embodiment, each R², taken together, forms a targeting sequence comprising SEQ ID NO: 70. In an embodiment, each R², taken together, forms a targeting sequence comprising SEQ ID NO: 74. In an embodiment, R², taken together, forms a targeting sequence comprising SEQ ID NO: 62. In an embodiment, each R², taken together, forms a targeting sequence comprising SEQ ID NO: 81. In an embodiment, each R², taken together, forms a targeting sequence comprising SEQ ID NO: 114. In an embodiment, each R², taken together, forms a targeting sequence comprising SEQ ID NO: 120.

In some embodiments, the antisense oligomer, or pharmaceutically acceptable salt thereof, of Formula (I), Formula (IA), Formula (II), Formula (III), and/or Formula (IV) is an antisense oligomer, or a pharmaceutically acceptable salt thereof, selected from:

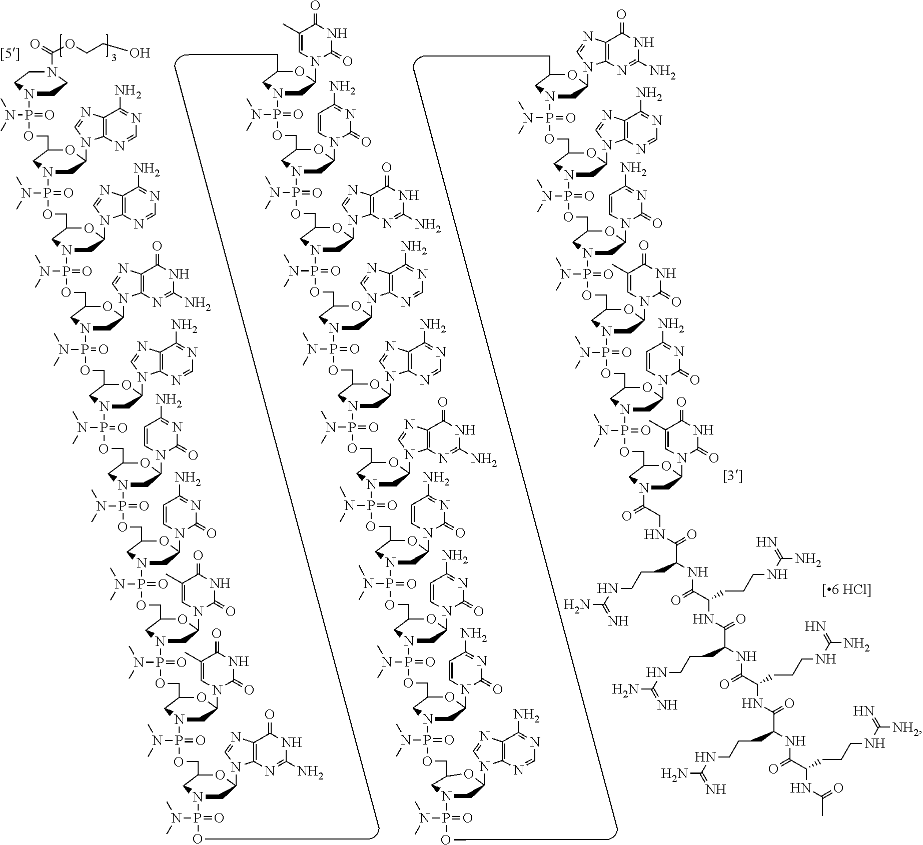

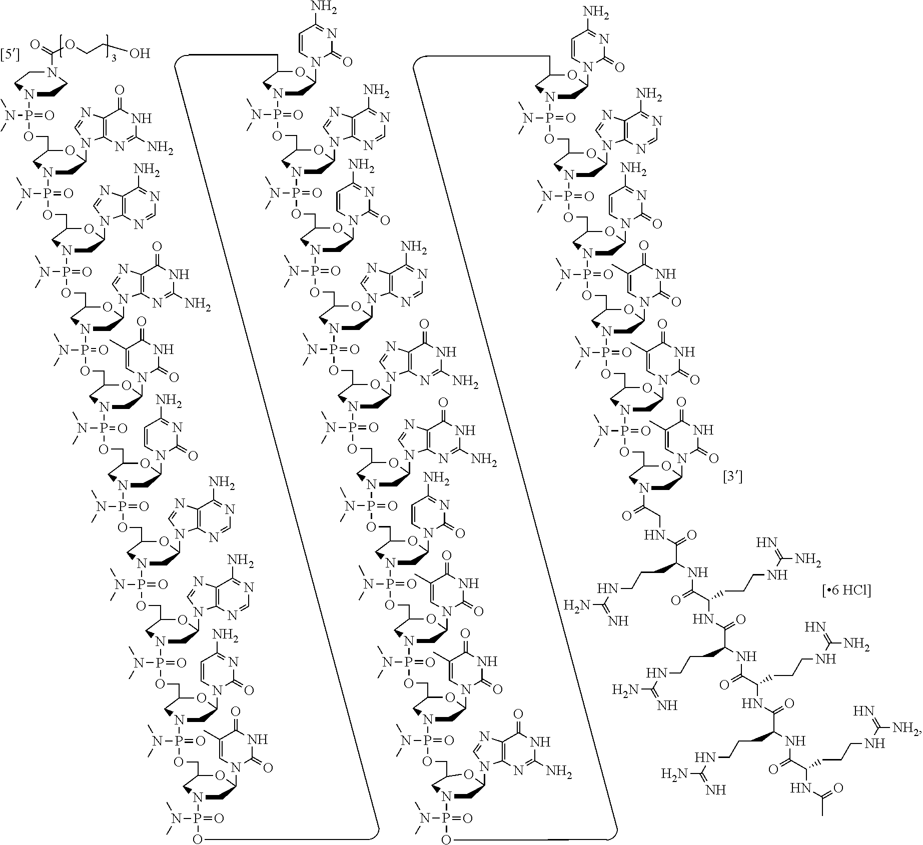

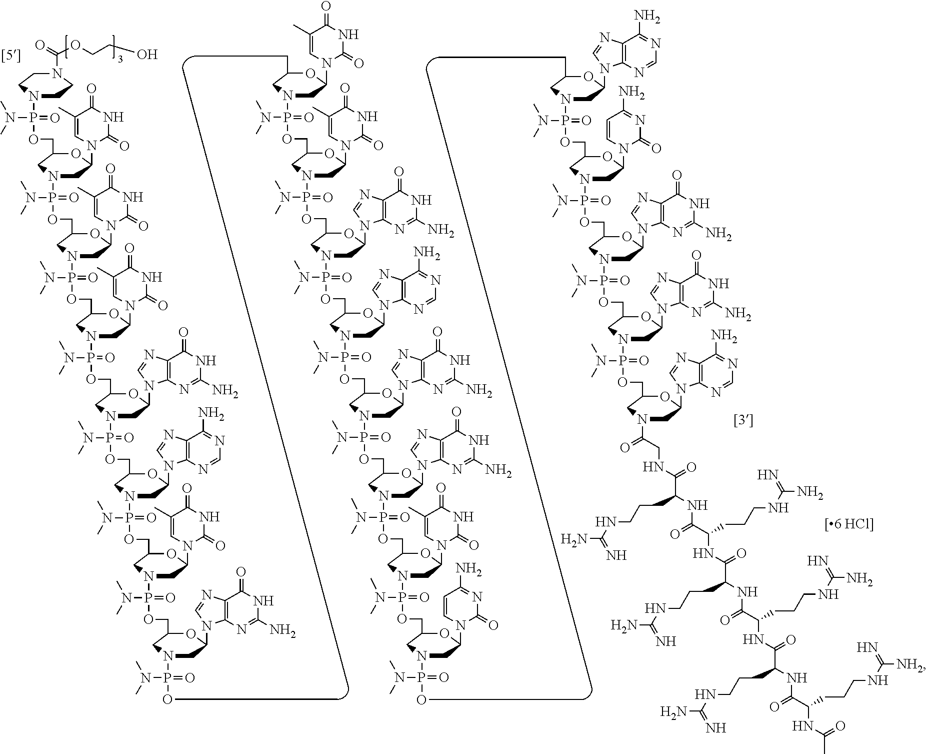

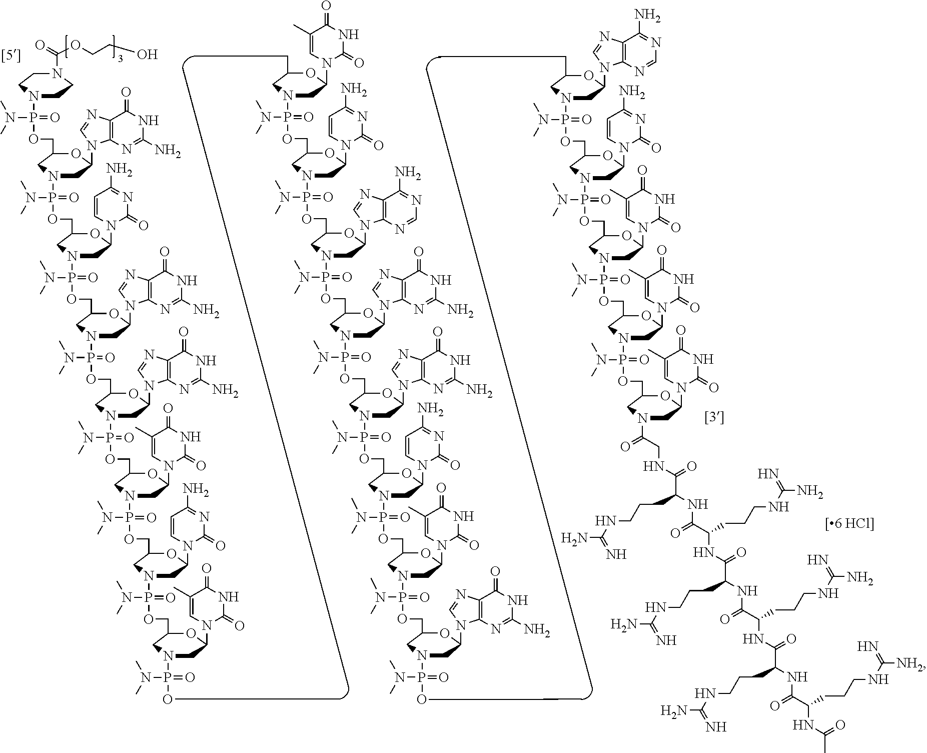

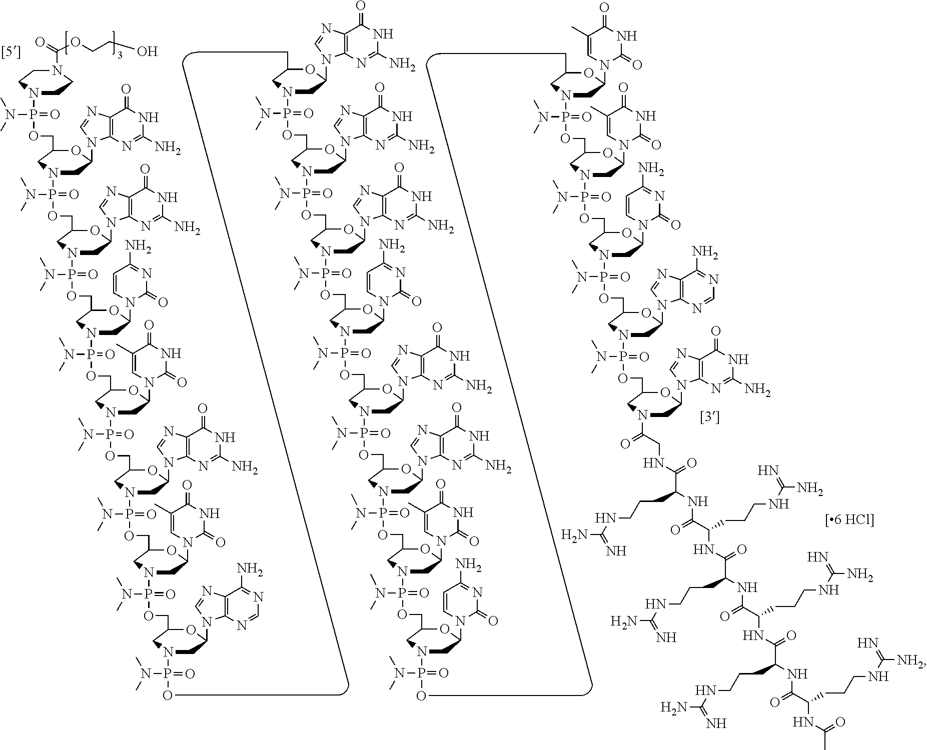

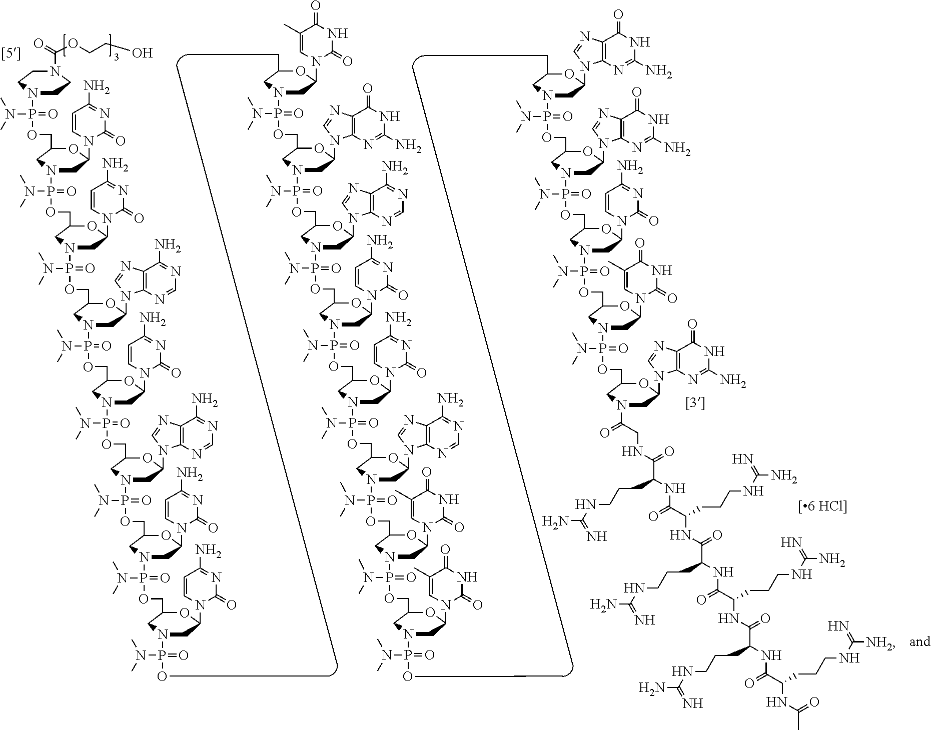

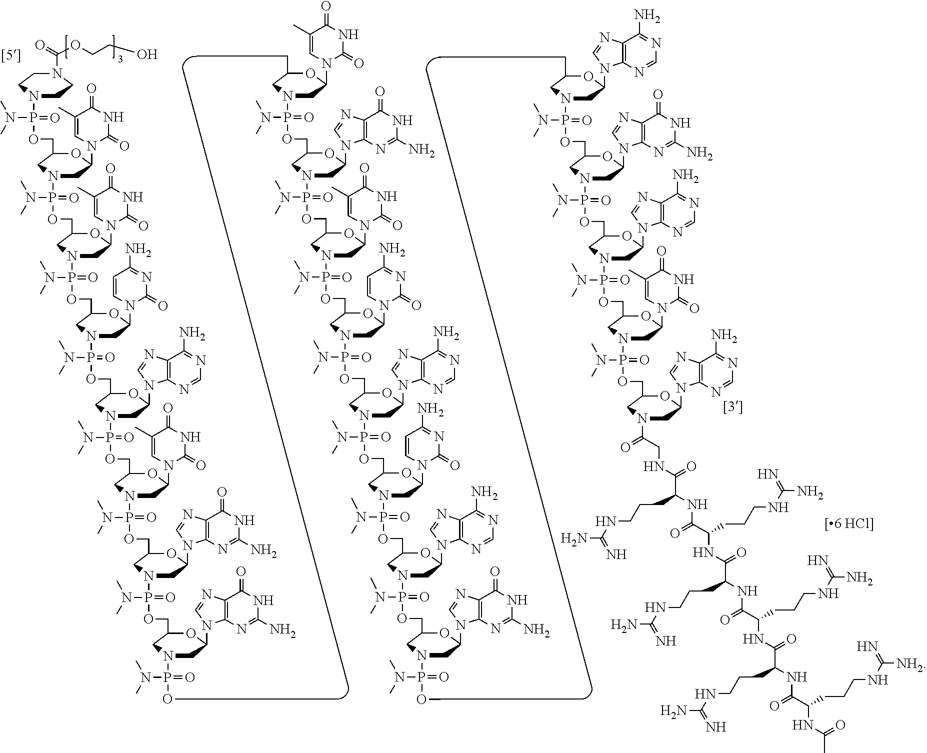

In an embodiment, the targeting sequence is complementary to a target region that is an intron/exon junction or exon internal region of exon 2 (SEQ ID NO: 2), exon 5 (SEQ ID NO: 3), exon 6 (SEQ ID NO: 4), exon 8 (SEQ ID NO:5), or exon 9 (SEQ ID NO: 6).

In an embodiment, the target region is an intron/exon junction or an exon internal region of exon 2. In certain embodiments, the target region is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175).

In another embodiment, the target region is an intron/exon junction or an exon internal region of exon 5. In certain embodiments, the target region is selected from H5A (−15+5), H5A (−14+6), H5A (−11+9), H5A (−10+10), H5A(+51+75), H5A(+76+100), H5A(+78+95), H5A(+80+97), H5A(+82+99), H5A(+126+150), H5A(+153+172), H5A(+154+173), H5D (+18-2), H5D (+16-4), H5D (+14-6), H5D (+13-7), and H5D (+12-8).

In another embodiment, the target region is an intron/exon junction or an exon internal region of exon 6. In certain embodiments, the target region is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D (+24-1), H6D (+15-5), and H6D (+14-6).

In yet another embodiment, the target region is an intron/exon junction or an exon internal region of exon 8. In certain embodiments, the target region is selected from H8A (−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D (+12-13).

In still another embodiment, the target region is an intron/exon junction or an exon internal region of exon 9. In certain embodiments, the target region is selected from H9A (−5+20), H9A(+1+25), H9A(+51+75), and H9D (+7-18).

In an embodiment, the targeting sequence comprises a sequence selected from:

SEQ ID NO: 11
(GTCTGGTGTCCTGTGAACAGAGATG);
SEQ ID NO: 12
(CTCTCTGTCTCTGATGTCTGGTGTC);
SEQ ID NO: 13
(AGACGGGTTGGCCCTTTGAATTTTT);
SEQ ID NO: 14
(TCTAGATAGCACCTGCCCAAAGGAA);
SEQ ID NO: 15
(ATCCTTTCTGCTCTTCCCGC);
SEQ ID NO: 16
(CATCCTTTCTGCTCTTCCCG);
SEQ ID NO: 17
(AGAGATGGCTGCCCCATCCTTTCTG);
SEQ ID NO: 20
(CAAGTCAGAGATGGCTGCCCCATCC);
SEQ ID NO: 23
(CATCCAAGTCAGAGATGGCT);
SEQ ID NO: 26
(ACCATCAGCATCCAAGTCAG);
SEQ ID NO: 27
(AGAGGCCACCACCACCATCAGCATC);
and
SEQ ID NO: 28
(CAGTGGCTGCAGTTGTGATGAACCA).

In another embodiment, the targeting sequence comprises a sequence selected from:

SEQ ID NO: 30
(GATATCTGAAACAGGTTAGG);
SEQ ID NO: 31
(AGATATCTGAAACAGGTTAG);
SEQ ID NO: 32
(GGGAGATATCTGAAACAGGT);
SEQ ID NO: 33
(AGGGAGATATCTGAAACAGG);
SEQ ID NO: 35
(TCAGCTGGCACTTGCCCAGCGACAC);
SEQ ID NO: 37
(AAGACCTTGTCGAAGCCCAGACTCT);
SEQ ID NO: 38
(CTTGTCGAAGCCCAGACT);
SEQ ID NO: 39
(ACCTTGTCGAAGCCCAGA);
SEQ ID NO: 40
(AGACCTTGTCGAAGCCCA);
SEQ ID NO: 44
(GGTTGTCTCTGTCATTGAAGCCCGA);
SEQ ID NO: 46
(GTCACTACAGACACCCAGTC);
SEQ ID NO: 47
(GGTCACTACAGACACCCAGT);
SEQ ID NO: 48
(ACCGTCAACACTGTCCCACA);
SEQ ID NO: 50
(GTACCGTCAACACTGTCCCA);
SEQ ID NO: 51
(ACGTACCGTCAACACTGTCC);
SEQ ID NO: 52
(GACGTACCGTCAACACTGTC);
and
SEQ ID NO: 53
(GGACGTACCGTCAACACTGT).

In a further embodiment, the targeting sequence comprises a sequence selected from:

SEQ ID NO: 57
(TCATCTGCCAGGTAGAGGGTGTTGC);
SEQ ID NO: 58
(GGTCACGGATGATGATCTCA);
SEQ ID NO: 59
(AGGTCACGGATGATGATCTC);
SEQ ID NO: 61
(TTGATGTTGAGGTCACGGAT);
SEQ ID NO: 62
(TTTGATGTTGAGGTCACGGA);
SEQ ID NO: 64
(GGTAGGAGCATGCAAAGTTGATTTT);
SEQ ID NO: 66
(TTCAGGCTGACTTTCATGTCCAGGG);
SEQ ID NO: 67
(GCTGACTTTCATGTCCAGGG);
SEQ ID NO: 68
(GGTCTTCAGGCTGACTTTCA);
SEQ ID NO: 69
(CGGTCTTCAGGCTGACTTTC);
SEQ ID NO: 70
(GCGGTCTTCAGGCTGACTTT);
SEQ ID NO: 71
(GGCGGTCTTCAGGCTGACTT);
SEQ ID NO: 72
(CTGTAGGGCGGTCTTCAGGC);
SEQ ID NO: 73
(GCTGTAGGGCGGTCTTCAGG);
SEQ ID NO: 74
(GGCTGTAGGGCGGTCTTCAG);
SEQ ID NO: 75
(TGGCTGTAGGGCGGTCTTCA);
SEQ ID NO: 76
(TTGGCTGTAGGGCGGTCTTC);
SEQ ID NO: 77
(ATTGGCTGTAGGGCGGTCTT);
SEQ ID NO: 78
(CTGACCATTGGCTGTAGGGC);
SEQ ID NO: 79
(CCTGACCATTGGCTGTAGGGCGGTC);
SEQ ID NO: 80
(CACACCTGACCATTGGCTGT);
and
SEQ ID NO: 81
(CCACACCTGACCATTGGCTG).

In yet another embodiment, the targeting sequence comprises a sequence selected from:

SEQ ID NO: 82
(TTGAGTCTCTAGTGTGTGGGCATCT);
SEQ ID NO: 84
(TGGACGGAAAATCGGCCCTGGGAGG);
SEQ ID NO: 85
(CATCTGGACGGAAAATCGGC);
SEQ ID NO: 86
(ACATCTGGACGGAAAATCGG);
SEQ ID NO: 89
(AACCGGAACATCTGGACGGA);
SEQ ID NO: 90
(AAACCGGAACATCTGGACGG);
SEQ ID NO: 91
(CAAACCGGAACATCTGGACG);
SEQ ID NO: 92
(TTCCAGCAAACCGGAACATC);
SEQ ID NO: 93
(ATAGTTTCCAGCAAACCGGAACATC);
SEQ ID NO: 94
(TTTCCAGCAAACCGGAACAT);
SEQ ID NO: 95
(GTTTCCAGCAAACCGGAACA);
SEQ ID NO: 96
(AGTTTCCAGCAAACCGGAAC);
SEQ ID NO: 98
(ATAGTTTCCAGCAAACCGGA);
SEQ ID NO: 99
(CATAGTTTCCAGCAAACCGG);
SEQ ID NO: 100
(TCATAGTTTCCAGCAAACCG);
SEQ ID NO: 101
(AGGTCATAGTTTCCAGCAAA);
SEQ ID NO: 102
(GGTAGACTAGGTCATAGTTT);
SEQ ID NO: 103
(AGGTAGACTAGGTCATAGTT);
SEQ ID NO: 104
(CAGGTAGACTAGGTCATAGT);
SEQ ID NO: 105
(CTTCACAGTGCAGGTAGACTAGGTC);
SEQ ID NO: 106
(ACAGTGCAGGTAGACTAGGT);
SEQ ID NO: 107
(CACAGTGCAGGTAGACTAGG);
SEQ ID NO: 108
(TCACAGTGCAGGTAGACTAG);
SEQ ID NO: 109
(TTCACAGTGCAGGTAGACTA);
SEQ ID NO: 110
(GTCACAGAGATAGACTTCAC);
SEQ ID NO: 111
(TGTCACAGAGATAGACTTCA);
SEQ ID NO: 112
(GTGTCACAGAGATAGACTTC);
SEQ ID NO: 113
(TCATTCATGGTGTCACAGAGATAGA);
SEQ ID NO: 114
(TTCATGGTGTCACAGAGATA);
SEQ ID NO: 115
(ATTCATGGTGTCACAGAGAT);
and
SEQ ID NO: 120
(GAGTCAACTCACAGGCTTGCACTTT).

In still another embodiment, the targeting sequence comprises a sequence selected from:

SEQ ID NO: 121
(AATCTGGTCCCAGAGCAGGTCTACA);
SEQ ID NO: 122
(TTCGGAATCTGGTCCCAGAGCAGGT);
SEQ ID NO: 123
(TGTGATGGGACCCAAGTTCAGGACA);
and
SEQ ID NO: 124
(AGCGAGTGGCTCTCTTACCTTTCCG).

In an embodiment, the antisense oligomer, or a pharmaceutically acceptable salt thereof, has a targeting sequence complementary to a region within an intron/exon junction or an exon internal junction of the human UMOD gene pre-mRNA, wherein the targeting sequence comprises a sequence selected from:

SEQ ID NO: 32
(GGGAGATATCTGAAACAGGT);
SEQ ID NO: 37
(AAGACCTTGTCGAAGCCCAGACTCT);
SEQ ID NO: 62
(TTTGATGTTGAGGTCACGGA);
SEQ ID NO: 70
(GCGGTCTTCAGGCTGACTTT);
SEQ ID NO: 74
(GGCTGTAGGGCGGTCTTCAG);
SEQ ID NO: 81
(CCACACCTGACCATTGGCTG);
SEQ ID NO: 114
(TTCATGGTGTCACAGAGATA);
and
SEQ ID NO: 120
(GAGTCAACTCACAGGCTTGCACTTT).

In an embodiment, the target region is an intron/exon junction or an exon internal region of exon 2. In certain embodiments, the target region is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175). In certain embodiments, the targeting sequence comprises a sequence selected from:

SEQ ID NO: 11
(GTCTGGTGTCCTGTGAACAGAGATG);
SEQ ID NO: 12
(CTCTCTGTCTCTGATGTCTGGTGTC);
SEQ ID NO: 13
(AGACGGGTTGGCCCTTTGAATTTTT);
SEQ ID NO: 14
(TCTAGATAGCACCTGCCCAAAGGAA);
SEQ ID NO: 15
(ATCCTTTCTGCTCTTCCCGC);
SEQ ID NO: 16
(CATCCTTTCTGCTCTTCCCG);
SEQ ID NO: 17
(AGAGATGGCTGCCCCATCCTTTCTG);
SEQ ID NO: 20
(CAAGTCAGAGATGGCTGCCCCATCC);
SEQ ID NO: 23
(CATCCAAGTCAGAGATGGCT);
SEQ ID NO: 26
(ACCATCAGCATCCAAGTCAG);
SEQ ID NO: 27
(AGAGGCCACCACCACCATCAGCATC);
and
SEQ ID NO: 28
(CAGTGGCTGCAGTTGTGATGAACCA).

In another embodiment, the target region is an intron/exon junction or an exon internal region of exon 5. In certain embodiments, the target region is selected from H5A (−15+5), H5A (−14+6), H5A (−11+9), H5A (−10+10), H5A(+51+75), H5A(+76+100), H5A(+78+95), H5A(+80+97), H5A(+82+99), H5A(+126+150), H5A(+153+172), H5A(+154+173), H5D (+18-2), H5D (+16-4), H5D (+14-6), H5D (+13-7), and H5D (+12-8). In certain embodiments, the targeting sequence comprises a sequence selected from:

SEQ ID NO: 30
(GATATCTGAAACAGGTTAGG);
SEQ ID NO: 31
(AGATATCTGAAACAGGTTAG);
SEQ ID NO: 32
(GGGAGATATCTGAAACAGGT);
SEQ ID NO: 33
(AGGGAGATATCTGAAACAGG);
SEQ ID NO: 35
(TCAGCTGGCACTTGCCCAGCGACAC);
SEQ ID NO: 37
(AAGACCTTGTCGAAGCCCAGACTCT);
SEQ ID NO: 38
(CTTGTCGAAGCCCAGACT);
SEQ ID NO: 39
(ACCTTGTCGAAGCCCAGA);
SEQ ID NO: 40
(AGACCTTGTCGAAGCCCA);
SEQ ID NO: 44
(GGTTGTCTCTGTCATTGAAGCCCGA);
SEQ ID NO: 46
(GTCACTACAGACACCCAGTC);
SEQ ID NO: 47
(GGTCACTACAGACACCCAGT);
SEQ ID NO: 48
(ACCGTCAACACTGTCCCACA);
SEQ ID NO: 50
(GTACCGTCAACACTGTCCCA);
SEQ ID NO: 51
(ACGTACCGTCAACACTGTCC);
SEQ ID NO: 52
(GACGTACCGTCAACACTGTC);
and
SEQ ID NO: 53
(GGACGTACCGTCAACACTGT).

In another embodiment, the target region is an intron/exon junction or an exon internal region of exon 6. In certain embodiments, the target region is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D (+24-1), H6D (+15-5), and H6D (+14-6). In certain embodiments, the targeting sequence comprises a sequence selected from:

SEQ ID NO: 57
(TCATCTGCCAGGTAGAGGGTGTTGC);
SEQ ID NO: 58
(GGTCACGGATGATGATCTCA);
SEQ ID NO: 59
(AGGTCACGGATGATGATCTC);
SEQ ID NO: 61
(TTGATGTTGAGGTCACGGAT);
SEQ ID NO: 62
(TTTGATGTTGAGGTCACGGA);
SEQ ID NO: 64
(GGTAGGAGCATGCAAAGTTGATTTT);
SEQ ID NO: 66
(TTCAGGCTGACTTTCATGTCCAGGG);
SEQ ID NO: 67
(GCTGACTTTCATGTCCAGGG);
SEQ ID NO: 68
(GGTCTTCAGGCTGACTTTCA);
SEQ ID NO: 69
(CGGTCTTCAGGCTGACTTTC);
SEQ ID NO: 70
(GCGGTCTTCAGGCTGACTTT);
SEQ ID NO: 71
(GGCGGTCTTCAGGCTGACTT);
SEQ ID NO: 72
(CTGTAGGGCGGTCTTCAGGC);
SEQ ID NO: 73
(GCTGTAGGGCGGTCTTCAGG);
SEQ ID NO: 74
(GGCTGTAGGGCGGTCTTCAG);
SEQ ID NO: 75
(TGGCTGTAGGGCGGTCTTCA);
SEQ ID NO: 76
(TTGGCTGTAGGGCGGTCTTC);
SEQ ID NO: 77
(ATTGGCTGTAGGGCGGTCTT);
SEQ ID NO: 78
(CTGACCATTGGCTGTAGGGC);
SEQ ID NO: 79
(CCTGACCATTGGCTGTAGGGCGGTC);
SEQ ID NO: 80
(CACACCTGACCATTGGCTGT);
and
SEQ ID NO: 81
(CCACACCTGACCATTGGCTG).

In yet another embodiment, the target region is an intron/exon junction or an exon internal region of exon 8. In certain embodiments, the target region is selected from H8A (−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D (+12-13). In certain embodiments, the targeting sequence comprises a

SEQ ID NO: 82
(TTGAGTCTCTAGTGTGTGGGCATCT);
SEQ ID NO: 84
(TGGACGGAAAATCGGCCCTGGGAGG);
SEQ ID NO: 85
(CATCTGGACGGAAAATCGGC);
SEQ ID NO: 86
(ACATCTGGACGGAAAATCGG);
SEQ ID NO: 89
(AACCGGAACATCTGGACGGA);
SEQ ID NO: 90
(AAACCGGAACATCTGGACGG);
SEQ ID NO: 91
(CAAACCGGAACATCTGGACG);
SEQ ID NO: 92
(TTCCAGCAAACCGGAACATC);
SEQ ID NO: 93
(ATAGTTTCCAGCAAACCGGAACATC);
SEQ ID NO: 94
(TTTCCAGCAAACCGGAACAT);
SEQ ID NO: 95
(GTTTCCAGCAAACCGGAACA);
SEQ ID NO: 96
(AGTTTCCAGCAAACCGGAAC);
SEQ ID NO: 98
(ATAGTTTCCAGCAAACCGGA);
SEQ ID NO: 99
(CATAGTTTCCAGCAAACCGG);
SEQ ID NO: 100
(TCATAGTTTCCAGCAAACCG);
SEQ ID NO: 101
(AGGTCATAGTTTCCAGCAAA);
SEQ ID NO: 102
(GGTAGACTAGGTCATAGTTT);
SEQ ID NO: 103
(AGGTAGACTAGGTCATAGTT);
SEQ ID NO: 104
(CAGGTAGACTAGGTCATAGT);
SEQ ID NO: 105
(CTTCACAGTGCAGGTAGACTAGGTC);
SEQ ID NO: 106
(ACAGTGCAGGTAGACTAGGT);
SEQ ID NO: 107
(CACAGTGCAGGTAGACTAGG);
SEQ ID NO: 108
(TCACAGTGCAGGTAGACTAG);
SEQ ID NO: 109
(TTCACAGTGCAGGTAGACTA);
SEQ ID NO: 110
(GTCACAGAGATAGACTTCAC);
SEQ ID NO: 111
(TGTCACAGAGATAGACTTCA);
SEQ ID NO: 112
(GTGTCACAGAGATAGACTTC);
SEQ ID NO: 113
(TCATTCATGGTGTCACAGAGATAGA);
SEQ ID NO: 114
(TTCATGGTGTCACAGAGATA);
SEQ ID NO: 115
(ATTCATGGTGTCACAGAGAT);
and
SEQ ID NO: 120
(GAGTCAACTCACAGGCTTGCACTTT).

In still another embodiment, the target region is an intron/exon junction or an exon internal region of exon 9. In certain embodiments, the target region is selected from H9A (−5+20), H9A(+1+25), H9A(+51+75), and H9D (+7-18). In certain embodiments, the targeting sequence comprises a sequence selected from:

SEQ ID NO: 121
(AATCTGGTCCCAGAGCAGGTCTACA);
SEQ ID NO: 122
(TTCGGAATCTGGTCCCAGAGCAGGT);
SEQ ID NO: 123
(TGTGATGGGACCCAAGTTCAGGACA);
and
SEQ ID NO: 124
(AGCGAGTGGCTCTCTTACCTTTCCG).

In an embodiment, provided herein is a pharmaceutical composition comprising an antisense oligomer described herein, or a pharmaceutically acceptable salt thereof, and at least one pharmaceutically acceptable carrier.

In another embodiment, provided herein is a method of treating a disease in a subject in need thereof, the method comprising administering a therapeutically effective amount of an antisense oligomer described herein, or a pharmaceutically acceptable salt thereof, or the pharmaceutical composition disclosed herein to the subject.

In an embodiment, the disease is chronic kidney disease (CKD). In an embodiment, the disease is associated with aberrant expression of uromodulin protein (UMOD). In an embodiment, the disease is uromodulin-associated renal disease. In an embodiment, the disease is an autosomal dominant renal disorder. In an embodiment, the autosomal dominant renal disorder is an autosomal dominant tubulointerstitial kidney disease (ADTKD). In an embodiment, the disease is uromodulin associated autosomal dominant tubulointerstitial kidney disease (ADTKD-UMOD), which is also known uromodulin kidney disease (UKD). In one embodiment, the subject is a human.

### III. Oligomer Chemistry Features

Provided herein are antisense oligomers, and pharmaceutically acceptable salts thereof, useful for targeting the human UMOD gene pre-mRNA, wherein the antisense oligomer, or a pharmaceutically acceptable salt thereof, is a non-natural (modified) antisense oligomer. Examples of modified antisense oligomers include, without limitation, morpholino oligomers, phosphorothioate modified oligomers, 2′-O-methyl modified oligomers, peptide nucleic acid (PNA), locked nucleic acid (LNA), phosphorothioate oligomers, 2′-O-MOE modified oligomers, 2′-fluoro-modified oligomer, 2′-0,4′-C-ethylene-bridged nucleic acids (ENAs), tricyclo-DNAs, tricyclo-DNA phosphorothioate subunits, 2′-O-[2-(N-methylcarbamoyl)ethyl] modified oligomers, including combinations of any of the foregoing. Phosphorothioate and 2′-O-Me-modified chemistries can be combined to generate a 2′-O-Me-phosphorothioate backbone. See, e.g., PCT Publication Nos. WO/2013/112053 and WO/2009/008725, each of which is incorporated herein by reference in its entirety.

In some embodiments, the nucleobases of the non-natural antisense oligomer, or of a pharmaceutically acceptable salt thereof, are linked to morpholino ring structures, wherein the morpholino ring structures are joined by phosphoros-containing intersubunit linkages joining a morpholino nitrogen of one ring structure to a 5′ exocyclic carbon of an adjacent ring structure.

In some embodiments, the nucleobases of the antisense oligomer, or of a pharmaceutically acceptable salt thereof, are linked to a peptide nucleic acid (PNA), wherein the phosphate-sugar polynucleotide backbone is replaced by a flexible pseudo-peptide polymer to which the nucleobases are linked. In some aspects, at least one of the nucleobases of the antisense oligomer, or of a pharmaceutically acceptable salt thereof, is linked to a locked nucleic acid (LNA), wherein the locked nucleic acid structure is a nucleotide analog that is chemically modified where the ribose moiety has an extra bridge connecting the 2′ oxygen and the 4′ carbon.

In some embodiments, at least one of the nucleobases of the antisense oligomer, or of a pharmaceutically acceptable salt thereof, is linked to a bridged nucleic acid (BNA), wherein the sugar conformation is restricted or locked by the introduction of an additional bridged structure to the furanose skeleton. In some aspects, at least one of the nucleobases of the antisense oligomer, or of a pharmaceutically acceptable salt thereof, is linked to a 2′-0,4′-C-ethylene-bridged nucleic acid (ENA).

In some embodiments, the non-natural antisense oligomer, or a pharmaceutically acceptable salt thereof, may contain unlocked nucleic acid (UNA) subunits. UNAs and UNA oligomers are an analog of RNA in which the C2′—C3′ bond of the subunit has been cleaved.

In some embodiments, the non-natural antisense oligomer, or a pharmaceutically acceptable salt thereof, contains one or more phosphorothioates (or S-oligos), in which one of the nonbridging oxygens is replaced by sulfur. In some aspects, the non-natural antisense oligomer, or a pharmaceutically acceptable salt thereof, contains one or more 2′ O-methyl, 2′ O-MOE, MCE, and 2′-F in which the 2′—OH of the ribose is substituted with a methyl, methoxy ethyl, 2-(N-methylcarbamoyl)ethyl, or fluoro group, respectively.

In some embodiments, the non-natural antisense oligomer, or a pharmaceutically acceptable salt thereof, is a tricyclo-DNA (tc-DNA) which is a constrained DNA analog in which each nucleotide is modified by the introduction of a cyclopropane ring to restrict conformational flexibility of the backbone and to optimize the backbone geometry of the torsion angle g.

In some embodiments, at least one of the nucleobases of the antisense oligomer, or a pharmaceutically acceptable salt thereof, is linked to a bridged nucleic acid (BNA), wherein the sugar conformation is restricted or locked by the introduction of an additional bridged structure to the furanose skeleton. In some aspects, at least one of the nucleobases of the antisense oligomer, or a pharmaceutically acceptable salt thereof, is linked to a 2′-0,4′-C-ethylene-bridged nucleic acid (ENA). In such aspects, each nucleobase which is linked to a BNA or ENA comprises a 5-methyl group. Exemplary embodiments of oligomer chemistries of the disclosure are further described below.

### 1. Peptide Nucleic Acids (PNAS)

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligomers obeying Watson-Crick base-pairing rules, and mimic DNA in terms of base-pair recognition. The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases. A non-limiting example of a PNA is depicted below.

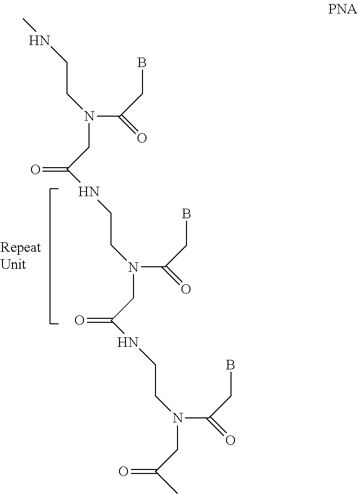

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration, and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling, and capping. PNAs can be produced synthetically using any technique known in the art. See, e.g., U.S. Pat. Nos. 6,969,766; 7,211,668; 7,022,851; 7,125,994; 7,145,006; and 7,179,896, each of which is incorporated herein by reference in its entirety. See also U.S. Pat. Nos. 5,539,082; 5,714,331; and 5,719,262 for the preparation of PNAs, each of which is incorporated herein by reference in its entirety. Further teaching of PNA compounds can be found in Nielsen et al., Science, 254:1497-1500, 1991, which is incorporated herein by reference in its entirety.

### 2. Locked Nucleic Acids (LNAs)

Antisense oligomers, and pharmaceutically acceptable salts thereof, may also contain “locked nucleic acid” subunits (LNAs). “LNAs” are a member of a class of modifications called bridged nucleic acid (BNA). BNA is characterized by a covalent linkage that locks the conformation of the ribose ring in a C₃₀-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Koshkin et al., Tetrahedron (1998) 54:3607; Jesper Wengel, Accounts of Chem. Research (1999) 32:301; Obika, et al., Tetrahedron Letters (1997) 38:8735; Obika, et al., Tetrahedron Letters (1998) 39:5401; and Obika, et al., Bioorganic Medicinal Chemistry (2008) 16:9230, each of which is incorporated herein by reference in its entirety. A non-limiting example of an LNA is depicted below.

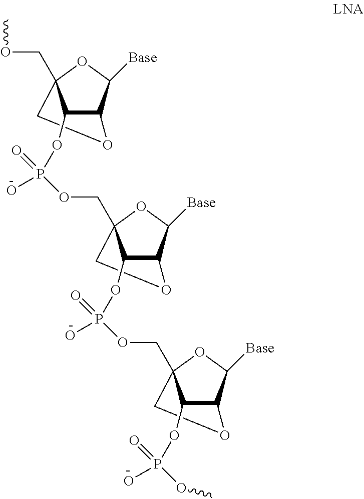

Antisense oligomers, and pharmaceutically acceptable salts thereof, of the disclosure may incorporate one or more LNAs; in some cases, the antisense oligomers, or pharmaceutically acceptable salts thereof, may be entirely composed of LNAs. Methods for the synthesis of individual LNA nucleoside subunits and their incorporation into oligomers are described, for example, in U.S. Pat. Nos. 7,572,582; 7,569,575; 7,084,125; 7,060,809; 7,053,207; 7,034,133; 6,794,499; and 6,670,461; each of which is incorporated herein by reference in its entirety. Typical intersubunit linkers include phosphodiester and phosphorothioate moieties; alternatively, non-phosphorus-containing linkers may be employed. Further embodiments include an LNA containing an antisense oligomer, or a pharmaceutically acceptable salt thereof, where each LNA subunit is separated by a DNA subunit. Certain antisense oligomers, or pharmaceutically acceptable salts thereof, are composed of alternating LNA and DNA subunits where the intersubunit linker is phosphorothioate.

### 3. Ethylene-Bridged Nucleic Acids (ENAs)

2′O,4′C-ethylene-bridged nucleic acids (ENAs) are another member of the class of BNAs. A non-limiting example is depicted below.

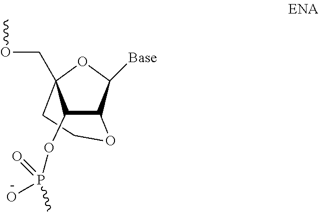

ENA oligomers and their preparation are described in Obika et al., Tetrahedron Lett (1997) 38 (50): 8735, which is incorporated herein by reference in its entirety. Antisense oligomers, or pharmaceutically acceptable salts thereof, of the disclosure may incorporate one or more ENA subunits.

### 4. Unlocked Nucleic Acid (UNA)

Antisense oligomers, or pharmaceutically acceptable salts thereof, may also contain unlocked nucleic acid (UNA) subunits. UNAs and UNA oligomers are an analog of RNA in which the C2′—C3′ bond of the subunit has been cleaved. Whereas LNA is conformationally restricted (relative to DNA and RNA), UNA is very flexible. UNAs are disclosed, for example, in WO 2016/070166. A non-limiting example of an UNA is depicted below.

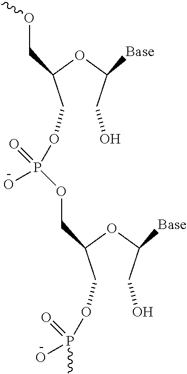

Typical intersubunit linkers include phosphodiester and phosphorothioate moieties; alternatively, non-phosphorus containing linkers may be employed.

### 5. Phosphorothioates

“Phosphorothioates” (or S-oligos) are a backbone variant of naturally occurring DNA in which one or more of the nonbridging oxygens is replaced by sulfur. A non-limiting example of a phosphorothioate is depicted below.

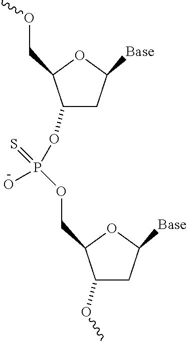

The sulfurization of the internucleotide bond reduces the action of endo- and exonucleases including 5′ to 3′ and 3′ to 5′ DNA POL 1 exonuclease, nucleases S1 and P1, RNases, serum nucleases, and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-benzodithiol-3-one 1, 1-dioxide (BDTD) (see, e.g., lyer et al., J. Org. Chem. 55, 4693-4699, 1990, which is incorporated herein by reference in its entirety). The latter methods avoid the problem of elemental sulfur's insolubility in most organic solvents and the toxicity of carbon disulfide. The TETD and BDTD methods also yield higher purity phosphorothioates.

### 6. Tricyclo-DNAs and Tricyclo-Phosphorothioate Subunits

Tricyclo-DNAs (tc-DNA) are a class of constrained DNA analogs in which each nucleotide is modified by the introduction of a cyclopropane ring to restrict conformational flexibility of the backbone and to optimize the backbone geometry of the torsion angle γ. Homobasic adenine- and thymine-containing tc-DNAs form extraordinarily stable A-T base pairs with complementary RNAs. Tricyclo-DNAs and their synthesis are described in International Patent Application Publication No. WO 2010/115993, which is hereby incorporated herein by reference in its entirety. Antisense oligomers, or pharmaceutically acceptable salts thereof, of the disclosure may incorporate one or more tricycle-DNA subunits; in some cases, the antisense oligomers, or pharmaceutically acceptable salts thereof, may be entirely composed of tricycle-DNA subunits.

Tricyclo-phosphorothioate subunits are tricyclo-DNA subunits with phosphorothioate intersubunit linkages. Tricyclo-phosphorothioate subunits and their synthesis are described in International Patent Application Publication No. WO 2013/053928, which is hereby incorporated herein by reference in its entirety. Antisense oligomers, or pharmaceutically acceptable salts thereof, of the disclosure may incorporate one or more tricycle-DNA subunits; in some cases, the antisense oligomers, or pharmaceutically acceptable salts thereof, may be entirely composed of tricycle-DNA subunits. A non-limiting example of a tricycle-DNA/tricycle-phosphorothioate subunit is depicted below.

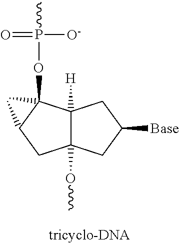

### 7. 2′—O-Methyl, 2′-O-MOE, and 2′-F Oligomers

“2′-O-Me oligomer” molecules carry a methyl group at the 2′—OH residue of the ribose molecule. 2′-O-Me-RNAs show the same (or similar) behavior as DNA but are protected against nuclease degradation. 2′-O-Me-RNAs can also be combined with phosphorothioate oligomers (PTOs) for further stabilization. 2′O-Me oligomers (phosphodiester or phosphorothioate) can be synthesized according to routine techniques in the art (see, e.g., Yoo et al., Nucleic Acids Res. 32:2008-16, 2004, which is hereby incorporated herein by reference in its entirety). A non-limiting example of a 2′-O-Me oligomer is depicted below.

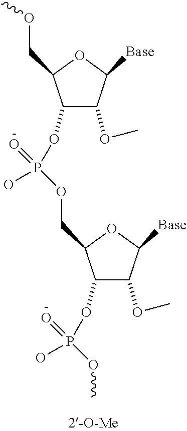

2′-O-Methoxyethyl oligomers (2′-O-MOE) carry a methoxyethyl group at the 2′—OH residue of the ribose molecule and are discussed in Martin et al., Helv. Chim. Acta, 78, 486-504, 1995, which is hereby incorporated herein by reference in its entirety. A non-limiting example of a 2′-O-MOE subunit is depicted below.

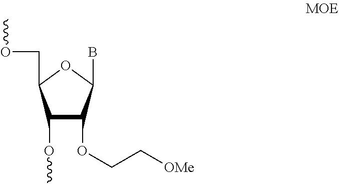

2′-Fluoro (2′-F) oligomers have a fluoro radical in at the 2′ position in place of the 2′—OH. A non-limiting example of a 2′-F oligomer is depicted below.

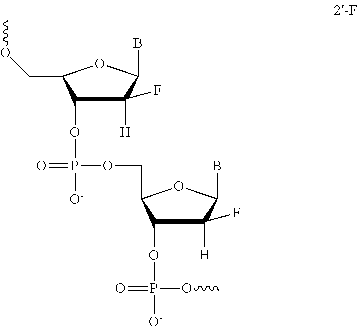

2′-Fluoro oligomers are further described in WO 2004/043977, which is hereby incorporated herein by reference in its entirety.

2′-O-Methyl, 2′-O-MOE, and 2′-F oligomers may also comprise one or more phosphorothioate (PS) linkages as depicted below.

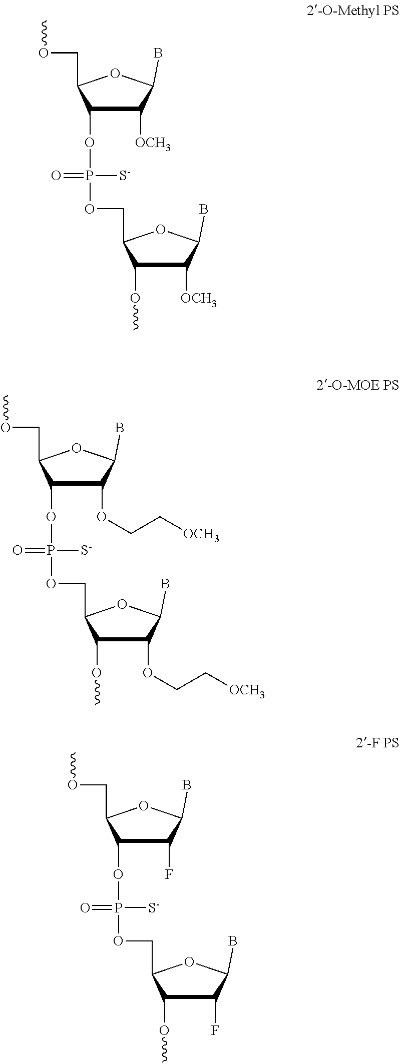

Additionally, 2′-O-methyl, 2′-O-MOE, and 2′-F oligomers may comprise PS intersubunit linkages throughout the oligomer, for example, as in the 2′-O-methyl PS oligomer drisapersen depicted below.

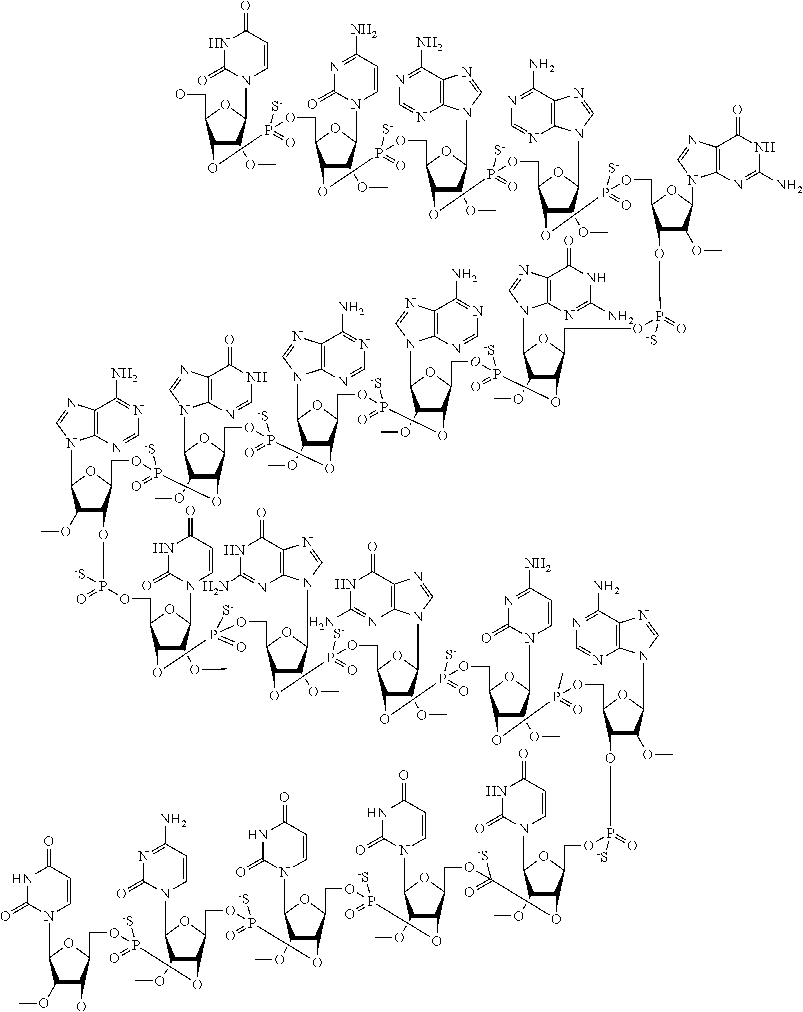

Alternatively, 2′-O-methyl, 2′-O-MOE, and/or 2′-F oligomers may comprise PS linkages at the ends of the oligomer, as depicted below:

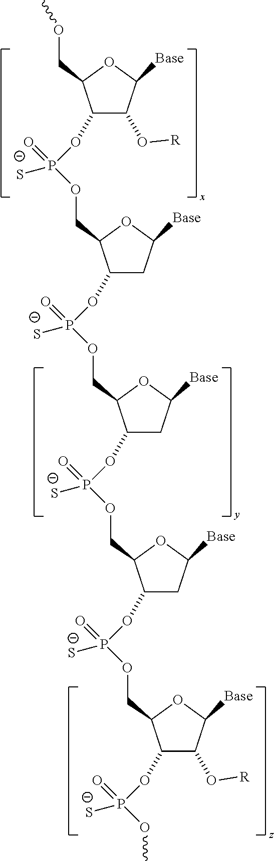

wherein:
R is CH₂CH₂OCH₃ (methoxyethyl or MOE); and X, Y, and Z denote the number of nucleotides contained within each of the designated 5′-wing, central gap, and 3′-wing regions, respectively.

Antisense oligomers, or pharmaceutically acceptable salts thereof, of the disclosure may incorporate one or more 2′-O-methyl, 2′-O-MOE, and 2′-F subunits and may utilize any of the intersubunit linkages described here. In some instances, an antisense oligomer, or a pharmaceutically acceptable salt thereof, of the disclosure may be composed of entirely 2′-O-methyl, 2′-O-MOE, or 2′-F subunits. One embodiment of the antisense oligomers, or pharmaceutically acceptable salts thereof, of the disclosure is composed entirely of 2′-O-methyl subunits.

### 8. 2′—O-[2-(N-methylcarbamoyl) ethyl] Oligomers (MCEs)

MCEs are another example of 2′-O modified ribonucleosides useful in the antisense oligomers, or pharmaceutically acceptable salts thereof, of the disclosure. Here, the 2′—OH is derivatized to a 2-(N-methylcarbamoyl)ethyl moiety to increase nuclease resistance. A non-limiting example of an MCE oligomer is depicted below.

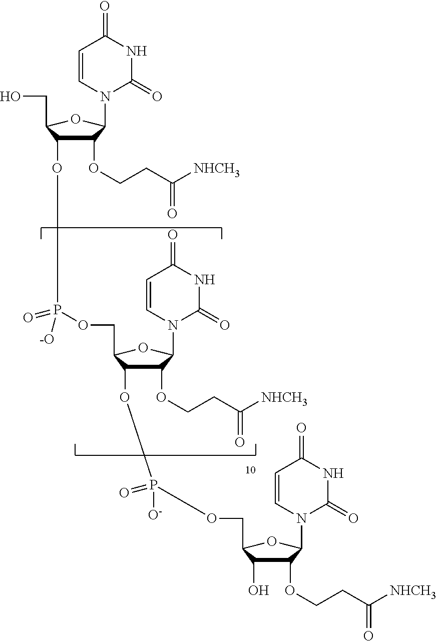

MCEs and their synthesis are described in Yamada et al., J. Org. Chem. (2011) 76 (9): 3042-53, which is hereby incorporated herein by reference in its entirety. Antisense oligomers, or pharmaceutically acceptable salts thereof, of the disclosure may incorporate one or more MCE subunits.

### 9. Stereo-Specific Oligomers

Stereo-specific oligomers are those in which the stereo chemistry of each phosphorus-containing linkage is fixed by the method of synthesis such that a substantially stereo-pure oligomer is produced. A non-limiting example of a stereo-specific oligomer is depicted below.

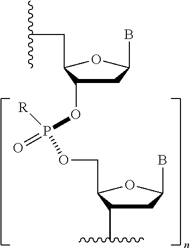

In the above example, each phosphorus of the oligomer has the same stereo configuration. Additional examples include the oligomers described herein. For example, LNAs, ENAs, Tricyclo-DNAs, MCEs, 2′-O-methyl, 2′-O-MOE, 2′-F, and morpholino-based oligomers can be prepared with stereo-specific phosphorus-containing internucleoside linkages such as, for example, phosphorothioate, phosphodiester, phosphoramidate, phosphorodiamidate, or other phosphorus-containing internucleoside linkages. Stereo specific oligomers, methods of preparation, chiral controlled synthesis, chiral design, and chiral auxiliaries for use in the preparation of such oligomers are detailed, for example, in WO2017192664, WO2017192679, WO2017062862, WO2017015575, WO2017015555, WO2015107425, WO2015108048, WO2015108046, WO2015108047, WO2012039448, WO2010064146, WO2011034072, WO2014010250, WO2014012081, WO20130127858, and WO2011005761, each of which is hereby incorporated herein by reference in its entirety.

Stereo-specific oligomers can have phosphorus-containing internucleoside linkages in an Rp or Sp configuration. Chiral phosphorus-containing linkages in which the stereo configuration of the linkages is controlled is referred to as “stereopure,” while chiral phosphorus-containing linkages in which the stereo configuration of the linkages is uncontrolled is referred to as “stereorandom.” In certain embodiments, the oligomers of the disclosure comprise a plurality of stereopure and stereorandom linkages, such that the resulting oligomer has stereopure subunits at pre-specified positions of the oligomer. An example of the location of the stereopure subunits is provided in international patent application publication number WO 2017/062862 A2 in FIGS. 7A and 7B. In an embodiment, all the chiral phosphorus-containing linkages in an oligomer are stereorandom. In an embodiment, all the chiral phosphorus-containing linkages in an oligomer are stereopure.

In an embodiment of an oligomer with “n” chiral phosphorus-containing linkages (where n is an integer of 1 or greater), all n of the chiral phosphorus-containing linkages in the oligomer are stereorandom. In an embodiment of an oligomer with n chiral phosphorus-containing linkages (where n is an integer of 1 or greater), all n of the chiral phosphorus-containing linkages in the oligomer are stereopure. In an embodiment of an oligomer with n chiral phosphorus-containing linkages (where n is an integer of 1 or greater), at least 10% (to the nearest integer) of the n phosphorus-containing linkages in the oligomer are stereopure. In an embodiment of an oligomer with n chiral phosphorus-containing linkages (where n is an integer of 1 or greater), at least 20% (to the nearest integer) of the n phosphorus-containing linkages in the oligomer are stereopure. In an embodiment of an oligomer with n chiral phosphorus-containing linkages (where n is an integer of 1 or greater), at least 30% (to the nearest integer) of the n phosphorus-containing linkages in the oligomer are stereopure. In an embodiment of an oligomer with n chiral phosphorus-containing linkages (where n is an integer of 1 or greater), at least 40% (to the nearest integer) of the n phosphorus-containing linkages in the oligomer are stereopure. In an embodiment of an oligomer with n chiral phosphorus-containing linkages (where n is an integer of 1 or greater), at least 50% (to the nearest integer) of the n phosphorus-containing linkages in the oligomer are stereopure. In an embodiment of an oligomer with n chiral phosphorus-containing linkages (where n is an integer of 1 or greater), at least 60% (to the nearest integer) of the n phosphorus-containing linkages in the oligomer are stereopure. In an embodiment of an oligomer with n chiral phosphorus-containing linkages (where n is an integer of 1 or greater), at least 70% (to the nearest integer) of the n phosphorus-containing linkages in the oligomer are stereopure. In an embodiment of an oligomer with n chiral phosphorus-containing linkages (where n is an integer of 1 or greater), at least 80% (to the nearest integer) of the n phosphorus-containing linkages in the oligomer are stereopure. In an embodiment of an oligomer with n chiral phosphorus-containing linkages (where n is an integer of 1 or greater), at least 90% (to the nearest integer) of the n phosphorus-containing linkages in the oligomer are stereopure.

In an embodiment of an oligomer with n chiral phosphorus-containing linkages (where n is an integer of 1 or greater), the oligomer contains at least 2 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 3 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 4 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 5 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 6 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 7 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 8 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 9 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 10 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 11 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 12 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 13 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 14 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 15 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 16 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 17 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 18 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 19 contiguous stereopure phosphorus-containing linkages of the same stereo orientation (i.e., either SP or RP). In an embodiment of an oligomer with n chiral phosphorus-containing linkages (where n is an integer of 1 or greater), the oligomer contains at least 20 contiguous stereopure phosphorus-containing linkages of the same stereo orientation (i.e., either SP or RP).

In an embodiment of an oligomer with n chiral phosphorus-containing linkages (where n is an integer of 1 or greater), the oligomer contains at least 2 contiguous stereopure phosphorus-containing linkages of the same stereo orientation (i.e., either SP or RP) and at least 2 contiguous stereopure phosphorus-containing linkages of the other stereo orientation. For example, the oligomer can contain at least 2 contiguous stereopure phosphorus-containing linkages of the SP orientation and at least 2 contiguous stereopure phosphorus-containing linkages of the RP orientation.

In an embodiment of an oligomer with n chiral phosphorus-containing linkages (where n is an integer of 1 or greater), the oligomer contains at least 2 contiguous stereopure phosphorus-containing linkages of the same stereo orientation in an alternating pattern. For example, the oligomer can contain the following in order: 2 or more RP, 2 or more SP, and 2 or more RP, etc.

### 10. Morpholino Oligomers

Exemplary embodiments of the disclosure relate to phosphorodiamidate morpholino oligomers of the following general structure:

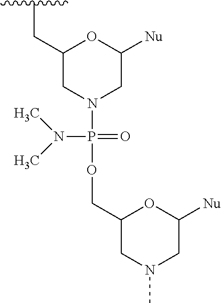

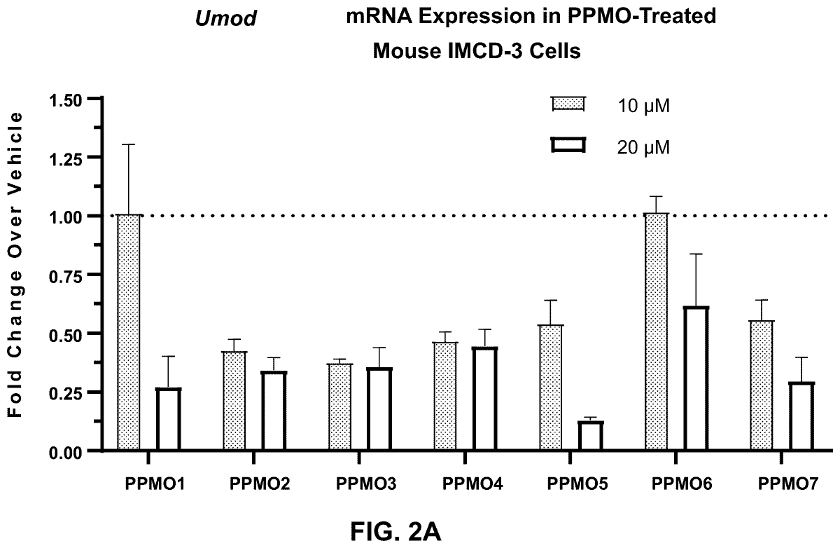

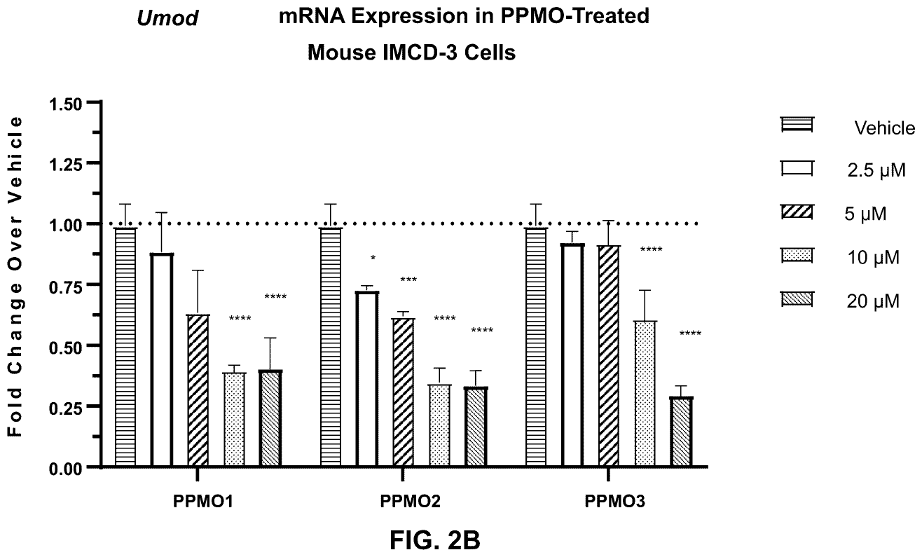

and as described in FIG. 2 of Summerton, J., et al., Antisense & Nucleic Acid Drug Development, 7:187-195 (1997). Morpholinos as described herein are intended to cover all stereoisomers and tautomers of the foregoing general structure. The synthesis, structures, and binding characteristics of morpholino oligomers are detailed in U.S. Pat. Nos. 5,698,685; 5,217,866; 5,142,047; 5,034,506; 5,166,315; 5,521,063; 5,506,337; 8,076,476; and 8,299,206, each of which are incorporated herein by reference in its entirety.

In certain embodiments, a morpholino is conjugated at 5′ or 3′ end of the oligomer with a “tail” moiety to increase its stability and/or solubility. Exemplary tails include:

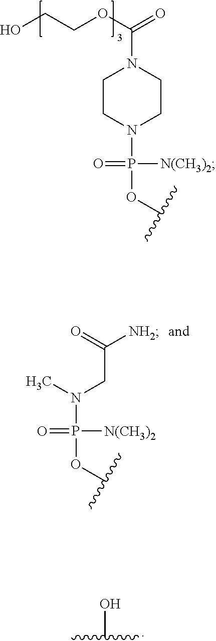

In various aspects, the disclosure provides antisense oligomers, or pharmaceutically acceptable salts thereof, of structural Formula (I), Formula (IA), Formula (II), Formula (III), and/or Formula (IV).

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), comprise a targeting sequence complementary to a target region within a pre-mRNA of the human UMOD gene. In another embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), comprise a targeting sequence complementary to a target region within a pre-mRNA of the human UMOD gene. In certain embodiments, the target region is an intron/exon junction or an exon internal region of exon 2 (SEQ ID NO: 2), exon 5 (SEQ ID NO: 3), exon 6 (SEQ ID NO: 4), exon 8 (SEQ ID NO: 5) or exon 9 (SEQ ID NO: 6).

In some embodiments of the antisense oligomers, or pharmaceutically acceptable salts thereof, of Formula (I), Formula (IA), Formula (II), Formula (III), and/or Formula (IV), the target region is an intron/exon junction of the human UMOD gene pre-mRNA. In other embodiments of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region is an exon internal region of the human UMOD gene pre-mRNA.

In an embodiment of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region is an intron/exon junction or an exon internal region of exon 2. In certain embodiments of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region of exon 2 is selected from H2A(−18+2), H2A(−17+3), H2A(−16+4), H2A(−15+10), H2A(+1+25), H2A(+26+50), H2A(+51+75), H2A(+85+104), H2A(+86+105), H2A(+95+119), H2A(+96+115), H2A(+98+117), H2A(+101+125), H2A(+108+127), H2A(+109+128), H2A(+110+129), H2A(+113+132), H2A(+114+133), H2A(+118+137), H2A(+126+150), H2A(+151+175), and H2D (+15-10). In some embodiments of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the targeting sequence comprises a sequence selected from SEQ ID NOs: 7-29.

In certain embodiments of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region of exon 2 is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175). In another embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region of exon 2, wherein the targeting sequence comprises or consists of a sequence selected from:

SEQ ID NO: 11
(GTCTGGTGTCCTGTGAACAGAGATG);
SEQ ID NO: 12
(CTCTCTGTCTCTGATGTCTGGTGTC);
SEQ ID NO: 13
(AGACGGGTTGGCCCTTTGAATTTTT);
SEQ ID NO: 14
(TCTAGATAGCACCTGCCCAAAGGAA);
SEQ ID NO: 15
(ATCCTTTCTGCTCTTCCCGC);
SEQ ID NO: 16
(CATCCTTTCTGCTCTTCCCG);
SEQ ID NO: 17
(AGAGATGGCTGCCCCATCCTTTCTG);
SEQ ID NO: 20
(CAAGTCAGAGATGGCTGCCCCATCC);
SEQ ID NO: 23
(CATCCAAGTCAGAGATGGCT);
SEQ ID NO: 26
(ACCATCAGCATCCAAGTCAG);
SEQ ID NO: 27
(AGAGGCCACCACCACCATCAGCATC);
and
SEQ ID NO: 28
(CAGTGGCTGCAGTTGTGATGAACCA).

In some embodiments, the targeting sequence is SEQ ID NO: 11. In some embodiments, the targeting sequence is SEQ ID NO: 12. In some embodiments, the targeting sequence is SEQ ID NO: 13. In some embodiments, the targeting sequence is SEQ ID NO: 14. In some embodiments, the targeting sequence is SEQ ID NO: 15. In some embodiments, the targeting sequence is SEQ ID NO: 16. In some embodiments, the targeting sequence is SEQ ID NO: 17. In some embodiments, the targeting sequence is SEQ ID NO: 20. In some embodiments, the targeting sequence is SEQ ID NO: 23. In some embodiments, the targeting sequence is SEQ ID NO: 26. In some embodiments, the targeting sequence is SEQ ID NO: 27. In some embodiments, the targeting sequence is SEQ ID NO: 28.

In some embodiments, the target region is H2A(−15+10). In some embodiments, the target region is H2A(+1+25). In some embodiments, the target region is H2A(+26+50). In some embodiments, the target region is H2A(+51+75). In some embodiments, the target region is H2A(+85+104). In some embodiments, the target region is H2A(+86+105). In some embodiments, the target region is H2A(+95+119). In some embodiments, the target region is H2A(+101+125). In some embodiments, the target region is H2A(+110+129). In some embodiments, the target region is H2A(+118+137). In some embodiments, the target region is H2A(+126+150). In some embodiments, the target region is H2A(+151+175).

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 51^{st} nucleotide to the 119^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 154. In other embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 51^{st} nucleotide to the 105^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 155. In still a further embodiment, the target region is H2A(+51+75). In certain embodiments, the antisense oligomer, or a pharmaceutically acceptable salt thereof, has a targeting sequence comprising or consisting of SEQ ID NO: 14.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 85^{th} nucleotide to the 119^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 156. In a further embodiment, the target region is selected from H2A(+85+104), H2A(+86+105), and H2A(+95+119). In still a further embodiment, the antisense oligomer, or a pharmaceutically acceptable salt thereof, has a targeting sequence comprising or consisting of a sequence selected from SEQ ID NOs: 15-17.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 15^{th} nucleotide of intron 1, as measured from the 5′ end of exon 2, and the 25^{th} nucleotide of exon 2, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 157. In a further embodiment, the target region is selected from H2A(−15+10) and H2A(+1+25). In some embodiments, the antisense oligomer, or a pharmaceutically acceptable salt thereof, has a targeting sequence comprising or consisting of SEQ ID NO: 12 or 13.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 118^{th} nucleotide to the 150^{th} nucleotide of exon 2, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 158. In a further embodiment, the target region is selected from H2A(+118+137) and H2A(+126+150). In certain embodiments, the antisense oligomers, or pharmaceutically acceptable salts thereof, have a targeting sequence comprising or consisting of SEQ ID NO: 26 or 27.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region of exon 2 selected from H2A(+101+125), H2A(+110+129), and H2A(+151+175). In a further embodiment, the antisense oligomer, or a pharmaceutically acceptable salt thereof, has a targeting sequence comprising or consisting of a sequence selected from SEQ ID NOs: 20, 21, and 28.

In another embodiment of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region is an intron/exon junction or an exon internal region of exon 5. In another embodiment of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region of exon 5 is selected from H5A (−15+5), H5A (−14+6), H5A (−11+9), H5A (−10+10), H5A (−9+11), H5A(+50+67), H5A(+51+75), H5A(+52+69), H5A(+76+100), H5A(+78+95), H5A(+80+97), H5A(+82+99), H5A(+85+102), H5A(+86+103), H5A(+91+108), H5A(+126+150), H5A(+152+171), H5A(+153+172), H5A(+154+173), H5D (+18-2), H5D (+17-3), H5D (+16-4), H5D (+14-6), H5D (+13-7), H5D (+12-8), H5D (+11-9), and H5D (+10-10). In some embodiments of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the antisense oligomer, or a pharmaceutically acceptable salt thereof, has a targeting sequence complementary to a target region of exon 5, wherein the targeting sequence comprises a sequence selected from SEQ ID NOs: 30-55.

In some embodiments of antisense oligomers, or pharmaceutically acceptable salts thereof, of Formula (I), Formula (IA), Formula (II), Formula (III), and/or Formula (IV), the target region of exon 5 is selected from H5A (−15+5), H5A (−14+6), H5A (−11+9), H5A (−10+10), H5A(+51+75), H5A(+76+100), H5A(+78+95), H5A(+80+97), H5A(+82+99), H5A(+126+150), H5A(+153+172), H5A(+154+173), H5D (+18-2), H5D (+16-4), H5D (+14-6), H5D (+13-7), and H5D (+12-8). In some embodiments, the antisense oligomers, or pharmaceutically acceptable salts thereof, disclosed herein, or a pharmaceutically acceptable salt thereof, including antisense oligomers, or pharmaceutically acceptable salts thereof, of Formula (I), Formula (IA), Formula (II), Formula (III), and/or Formula (IV), have a targeting sequence complementary to a target region of exon 5, wherein the targeting sequence comprises or consists of a sequence selected from:

SEQ ID NO: 30
(GATATCTGAAACAGGTTAGG);
SEQ ID NO: 31
(AGATATCTGAAACAGGTTAG);
SEQ ID NO: 32
(GGGAGATATCTGAAACAGGT);
SEQ ID NO: 33
(AGGGAGATATCTGAAACAGG);
SEQ ID NO: 35
(TCAGCTGGCACTTGCCCAGCGACAC);
SEQ ID NO: 37
(AAGACCTTGTCGAAGCCCAGACTCT);
SEQ ID NO: 38
(CTTGTCGAAGCCCAGACT);
SEQ ID NO: 39
(ACCTTGTCGAAGCCCAGA);
SEQ ID NO: 40
(AGACCTTGTCGAAGCCCA);
SEQ ID NO: 44
(GGTTGTCTCTGTCATTGAAGCCCGA);
SEQ ID NO: 46
(GTCACTACAGACACCCAGTC);
SEQ ID NO: 47
(GGTCACTACAGACACCCAGT);
SEQ ID NO: 48
(ACCGTCAACACTGTCCCACA);
SEQ ID NO: 50
(GTACCGTCAACACTGTCCCA);
SEQ ID NO: 51
(ACGTACCGTCAACACTGTCC);
SEQ ID NO: 52
(GACGTACCGTCAACACTGTC);
and
SEQ ID NO: 53
(GGACGTACCGTCAACACTGT).

In some embodiments, the targeting sequence is SEQ ID NO: 30. In some embodiments, the targeting sequence is SEQ ID NO: 31. In some embodiments, the targeting sequence is SEQ ID NO: 32. In some embodiments, the targeting sequence is SEQ ID NO: 33. In some embodiments, the targeting sequence is SEQ ID NO: 35. In some embodiments, the targeting sequence is SEQ ID NO: 37. In some embodiments, the targeting sequence is SEQ ID NO: 38. In some embodiments, the targeting sequence is SEQ ID NO: 39. In some embodiments, the targeting sequence is SEQ ID NO: 40. In some embodiments, the targeting sequence is SEQ ID NO: 44. In some embodiments, the targeting sequence is SEQ ID NO: 46. In some embodiments, the targeting sequence is SEQ ID NO: 47. In some embodiments, the targeting sequence is SEQ ID NO: 48. In some embodiments, the targeting sequence is SEQ ID NO: 50. In some embodiments, the targeting sequence is SEQ ID NO: 51. In some embodiments, the targeting sequence is SEQ ID NO: 52. In some embodiments, the targeting sequence is SEQ ID NO: 53.

In some embodiments, the target region is H5A (−15+5). In some embodiments, the target region is H5A (−14+6). In some embodiments, the target region is H5A (−11+9). In some embodiments, the target region is H5A (−10+10). In some embodiments, the target region is H5A(+51+75). In some embodiments, the target region is H5A(+76+100). In some embodiments, the target region is H5A(+78+95). In some embodiments, the target region is H5A(+80+97). In some embodiments, the target region is H5A(+82+99). In some embodiments, the target region is H5A(+126+150). In some embodiments, the target region is H5A(+153+172). In some embodiments, the target region is H5A(+154+173). In some embodiments, the target region is H5D (+18-2). In some embodiments, the target region is H5D (+16-4). In some embodiments, the target region is H5D (+14-6). In some embodiments, the target region is H5D (+13-7). In some embodiments, the target region is H5D (+12-8).

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 15^{th} nucleotide of intron 4, as measured from the 5′ end of exon 5 and the 10^{th} nucleotide of exon 5, as measured from the 5′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 159. In a further embodiment, the target region is selected from H5A (−15+5), H5A (−14+6), H5A (−11+9), and H5A (−10+10). In still a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising or consisting of a sequence selected from SEQ ID NOs: 30-33.

In an embodiment, the target region is H5A (−15+5). In another embodiment, the targeting sequence comprises SEQ ID NO: 30.

In an embodiment, the target region is H5A (−14+6). In another embodiment, the target region is H5A (−15+5). In certain embodiments the targeting sequence comprises SEQ ID NO: 31.

In an embodiment, the target region is H5A (−11+9). In another embodiment, the target region is H5A (−11+9). In some embodiments, the targeting sequence comprises SEQ ID NO: 32.

In an embodiment, the target region is H5A (−10+10). In another embodiment, the targeting sequence comprises SEQ ID NO: 33.

In an embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to the target region H5A(+51+75). In a further embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the targeting sequence comprises SEQ ID NO: 35.

In an embodiment, antisense oligomers, or pharmaceutically acceptable salts thereof, of the disclosed herein, including antisense oligomers, or pharmaceutically acceptable salts thereof, of Formula (I), Formula (IA), Formula (II), Formula (III), and/or Formula (IV), have a targeting sequence complementary to a target region within the 76^{th} nucleotide to the 100^{th} nucleotide, as measured from the 5′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 37. In a further embodiment, the target region is selected from H5A(+76+100), H5A(+78+95), H5A(+80+97), and H5A(+82+99). In one embodiment, the target region is H5A(+76+100). In another embodiment, the targeting sequence comprises SEQ ID NO: 37.

In one embodiment, the target region is H5A(+78+95). In another embodiment, the antisense oligomer, or a pharmaceutically acceptable salt thereof, has a targeting sequence comprising SEQ ID NO: 38.

In one embodiment, the target region is H5A(+80+97). In another embodiment, the antisense oligomer, or a pharmaceutically acceptable salt thereof, has a targeting sequence comprising SEQ ID NO: 39.

In one embodiment, the target region is H5A(+82+99). In another embodiment, the antisense oligomer, or a pharmaceutically acceptable salt thereof, has a targeting sequence comprising SEQ ID NO: 40.

In an embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 18^{th} nucleotide of exon 5 measured from 3′ end of exon 5 to the 8^{th} nucleotide of intron 5 measured from 3′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 160. In a further embodiment, the target region is selected from H5D (+18-2), H5D (+16-4), H5D (+14-6), H5D (+13-7), and H5D (+12-8). In still a further embodiment, the antisense oligomers, or pharmaceutically acceptable salts thereof, of Formula (I), Formula (IA), Formula (II), Formula (III), and/or Formula (IV), have a targeting sequence comprising a sequence selected from SEQ ID NOs: 48 and 50-53.

In another embodiment of antisense oligomers, or a pharmaceutically acceptable salt thereof, of Formula (I), Formula (IA), Formula (II), Formula (III), and/or Formula (IV), the target region is an intron/exon junction or an exon internal region of exon 6. In some embodiments of antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region of exon 6 is selected from H6A(+1+25), H6A(+26+50), H6A(+48+67), H6A(+49+68), H6A(+50+69), H6A(+58+77), H6A(+59+78), H6A(+60+79), H6A(+76+100), H6A(+79+98), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D (+24-1), H6D (+15-5), and H6D (+14-6). In some embodiments embodiment of antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the antisense oligomer, or a pharmaceutically acceptable salt thereof, has a targeting sequence complementary to a target region of exon 6, wherein the targeting sequence comprises a sequence selected from SEQ ID NOs: 56-81.

In certain embodiments, the target region of exon 6 is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D (+24-1), H6D (+15-5), and H6D (+14-6). In some embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region of exon 6, wherein the targeting sequence comprises or consists of a sequence selected from:

SEQ ID NO: 57
(TCATCTGCCAGGTAGAGGGTGTTGC);
SEQ ID NO: 58
(GGTCACGGATGATGATCTCA);
SEQ ID NO: 59
(AGGTCACGGATGATGATCTC);
SEQ ID NO: 61
(TTGATGTTGAGGTCACGGAT);
SEQ ID NO: 62
(TTTGATGTTGAGGTCACGGA);
SEQ ID NO: 64
(GGTAGGAGCATGCAAAGTTGATTTT);
SEQ ID NO: 66
(TTCAGGCTGACTTTCATGTCCAGGG);
SEQ ID NO: 67
(GCTGACTTTCATGTCCAGGG);
SEQ ID NO: 68
(GGTCTTCAGGCTGACTTTCA);
SEQ ID NO: 69
(CGGTCTTCAGGCTGACTTTC);
SEQ ID NO: 70
(GCGGTCTTCAGGCTGACTTT);
SEQ ID NO: 71
(GGCGGTCTTCAGGCTGACTT);
SEQ ID NO: 72
(CTGTAGGGCGGTCTTCAGGC);
SEQ ID NO: 73
(GCTGTAGGGCGGTCTTCAGG);
SEQ ID NO: 74
(GGCTGTAGGGCGGTCTTCAG);
SEQ ID NO: 75
(TGGCTGTAGGGCGGTCTTCA);
SEQ ID NO: 76
(TTGGCTGTAGGGCGGTCTTC);
SEQ ID NO: 77
(ATTGGCTGTAGGGCGGTCTT);
SEQ ID NO: 78
(CTGACCATTGGCTGTAGGGC);
SEQ ID NO: 79
(CCTGACCATTGGCTGTAGGGCGGTC);
SEQ ID NO: 80
(CACACCTGACCATTGGCTGT);
and
SEQ ID NO: 81
(CCACACCTGACCATTGGCTG).

In some embodiments, the targeting sequence is SEQ ID NO: 57. In some embodiments, the targeting sequence is SEQ ID NO: 58. In some embodiments, the targeting sequence is SEQ ID NO: 59. In some embodiments, the targeting sequence is SEQ ID NO: 61. In some embodiments, the targeting sequence is SEQ ID NO: 62. In some embodiments, the targeting sequence is SEQ ID NO: 64. In some embodiments, the targeting sequence is SEQ ID NO: 66. In some embodiments, the targeting sequence is SEQ ID NO: 67. In some embodiments, the targeting sequence is SEQ ID NO: 68. In some embodiments, the targeting sequence is SEQ ID NO: 69. In some embodiments, the targeting sequence is SEQ ID NO: 70. In some embodiments, the targeting sequence is SEQ ID NO: 71. In some embodiments, the targeting sequence is SEQ ID NO: 72. In some embodiments, the targeting sequence is SEQ ID NO: 73. In some embodiments, the targeting sequence is SEQ ID NO: 74. In some embodiments, the targeting sequence is SEQ ID NO: 75. In some embodiments, the targeting sequence is SEQ ID NO: 76. In some embodiments, the targeting sequence is SEQ ID NO: 77. In some embodiments, the targeting sequence is SEQ ID NO: 78. In some embodiments, the targeting sequence is SEQ ID NO: 79. In some embodiments, the targeting sequence is SEQ ID NO: 80. In some embodiments, the targeting sequence is SEQ ID NO: 81.

In some embodiments, the target region is H6A(+26+50). In some embodiments, the target region is H6A(+48+67). In some embodiments, the target region is H6A(+49+68). In some embodiments, the target region is H6A(+58+77). In some embodiments, the target region is H6A(+59+78). In some embodiments, the target region is H6A(+76+100). In some embodiments, the target region is H6A(+101+125). In some embodiments, the target region is H6A(+101+120). In some embodiments, the target region is H6A(+110+129). In some embodiments, the target region is H6A(+111+130). In some embodiments, the target region is H6A(+112+131). In some embodiments, the target region is H6A(+113+132). In some embodiments, the target region is H6A(+119+138). In some embodiments, the target region is H6A(+120+139). In some embodiments, the target region is H6A(+121+140). In some embodiments, the target region is H6A(+122+141). In some embodiments, the target region is H6A(+123+142). In some embodiments, the target region is H6A(+124+143). In some embodiments, the target region is H6A(+130+149). In some embodiments, the target region is H6D (+24-1). In some embodiments, the target region is H6D (+15-5). In some embodiments, the target region is H6D (+14-6).

In an embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to the target region H6A(+26+50). In a further embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the targeting sequence comprises SEQ ID NO: 57.

In an embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 48^{th} nucleotide to the 78^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 161. In a further embodiment, the target region is selected from H6A(+48+67), H6A(+49+68), H6A(+58+77), and H6A(+59+78). In still a further embodiment, the antisense oligomer, or a pharmaceutically acceptable salt thereof, has a targeting sequence comprising a sequence selected from SEQ ID NOs: 58, 59, 61, and 62.

In another embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 58^{th} nucleotide to the 78^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 162. In certain embodiments, the target region is selected from H6A(+58+77) and H6A(+59+78). In one embodiment, the target region is H6A(+58+77). In one embodiment, the antisense oligomer, or a pharmaceutically acceptable salt thereof, has a targeting sequence comprising SEQ ID NO: 61. In another embodiment, the target region is H6A(+59+78). In one embodiment, the antisense oligomer, or a pharmaceutically acceptable salt thereof, has a targeting sequence comprising SEQ ID NO: 62.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to the target region H6A(+76+100). In a further embodiment, the antisense oligomer, or a pharmaceutically acceptable salt thereof, has a targeting sequence complementary to the target region H6A(+26+50). In another embodiment, the targeting sequence comprises SEQ ID NO: 64.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 101^{st} nucleotide to the 132^{nd} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 163. In a further embodiment, the target region is selected from H6A(+101+125), H6A(+101+120), H6A(+110+129), H6A(+111+130), H6A(+112+131), and H6A(+113+132). In still a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising a sequence selected from SEQ ID NOs: 66-71.

In another embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 111^{th} nucleotide to the 132^{nd} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 164. In certain embodiments, the target region is selected from H6A(+111+130), H6A(+112+131), and H6A(+113+132). In one embodiment, the target region is H6A(+111+130). In one embodiment, the antisense oligomer, or a pharmaceutically acceptable salt thereof, has a targeting sequence comprising SEQ ID NO: 69. In another embodiment, the target region is H6A(+112+131). In certain embodiments and the antisense oligomer, or a pharmaceutically acceptable salt thereof, has a targeting sequence comprising SEQ ID NO: 70. In yet another embodiment, the target region is H6A(+113+133). In certain embodiments, the antisense oligomer, or a pharmaceutically acceptable salt thereof, has a targeting sequence comprising SEQ ID NO: 71.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 119^{th} nucleotide to the 149^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 165. In another embodiment, the target region is selected from H6A(+119+138), H6A(+120+139), H6A(+121+140), H6A(+122+141), H6A(+123+142), H6A(+124+143), and H6A(+130+149). In yet another embodiment, the antisense oligomer, or a pharmaceutically acceptable salt thereof, has a targeting sequence comprising a sequence selected from SEQ ID NOs: 72-78.

In a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 119^{th} nucleotide to the 141^{st} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 166. In an embodiment, the target region is selected from H6A(+119+138), H6A(+120+139), H6A(+121+140), and H6A(+122+141). In another embodiment, the antisense oligomer, or a pharmaceutically acceptable salt thereof, has a targeting sequence comprising a sequence selected from SEQ ID NOs: 72-75.

In still a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 120^{th} nucleotide to the 141^{st} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 167. In some embodiments, the target region is H6A(+120+139), H6A(+121+140), or H6A(+122+141). In one embodiment, the target region is H6A(+120+139). In another embodiment, the antisense oligomer, or a pharmaceutically acceptable salt thereof, has a targeting sequence comprising SEQ ID NO: 73. In another embodiment, the target region is H6A(+121+140). In certain embodiments, the antisense oligomer, or a pharmaceutically acceptable salt thereof, has a targeting sequence comprising SEQ ID NO: 74. In yet another embodiment, the target region is H6A(+122+141). In certain embodiments, the antisense oligomer, or a pharmaceutically acceptable salt thereof, has a targeting sequence comprising SEQ ID NO: 75.

In still a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 123^{rd} nucleotide to the 149^{th} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 168. In an embodiment, the target region is selected from H6A(+123+142), H6A(+124+143), and H6A(+130+149). In another embodiment, the antisense oligomer, or a pharmaceutically acceptable salt thereof, has a targeting sequence comprising a sequence selected from SEQ ID NOs: 76-78.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 24^{th} nucleotide of exon 6 measured from 3′ end of exon 6 to the 6^{th} nucleotide of intron 6 measured from 3′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 169. In a further embodiment, the target region is selected from H6D (+24-1), H6D (+15-5), and H6D (+14-6). In still a further embodiment, the antisense oligomer, or a pharmaceutically acceptable salt thereof, has a targeting sequence comprising a sequence selected from SEQ ID NOs: 79-81.

In a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 15^{th} nucleotide of exon 6 measured from 3′ end of exon 6 to the 6^{th} nucleotide of intron 6 measured from 3′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 170. In some embodiments, the target region is H6D (+15-5) or H6D (+14-6). In one embodiment, the target region is H6D (+15-5). In certain embodiments, the antisense oligomer, or a pharmaceutically acceptable salt thereof, has a targeting sequence comprising SEQ ID NO: 80. In another embodiment, the target region is H6D (+14-6). In certain embodiments, the antisense oligomer, or a pharmaceutically acceptable salt thereof, has a targeting sequence comprising SEQ ID NO: 81.

In yet another embodiment of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region is an intron/exon junction or an exon internal region of exon 8. In some embodiments of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region of exon 8 is selected from H8A (−2+23), H8A(+26+50), H8A(+51+75), H8A(+60+79), H8A(+61+80), H8A(+62+81), H8A(+63+82), H8A(+68+87), H8A(+69+88), H8A(+70+89), H8A(+76+95), H8A(+76+100), H8A(+77+96), H8A(+78+97), H8A(+79+98), H8A(+80+99), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), H8A(+133+152), H8A(+134+153), H8A(+139+158), H8D (+19-1), and H8D (+12-13). In some embodiments of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the targeting sequence comprises a sequence selected from SEQ ID NOs: 82-120.

In certain embodiments, the target region of exon 8 is selected from H8A (−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D (+12-13). In some embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region of exon 8, wherein the targeting sequence comprises a sequence selected from:

SEQ ID NO: 82
(TTGAGTCTCTAGTGTGTGGGCATCT);
SEQ ID NO: 84
(TGGACGGAAAATCGGCCCTGGGAGG);
SEQ ID NO: 85
(CATCTGGACGGAAAATCGGC);
SEQ ID NO: 86
(ACATCTGGACGGAAAATCGG);
SEQ ID NO: 89
(AACCGGAACATCTGGACGGA);
SEQ ID NO: 90
(AAACCGGAACATCTGGACGG);
SEQ ID NO: 91
(CAAACCGGAACATCTGGACG);
SEQ ID NO: 92
(TTCCAGCAAACCGGAACATC);
SEQ ID NO: 93
(ATAGTTTCCAGCAAACCGGAACATC);
SEQ ID NO: 94
(TTTCCAGCAAACCGGAACAT);
SEQ ID NO: 95
(GTTTCCAGCAAACCGGAACA);
SEQ ID NO: 96
(AGTTTCCAGCAAACCGGAAC);
SEQ ID NO: 98
(ATAGTTTCCAGCAAACCGGA);
SEQ ID NO: 99
(CATAGTTTCCAGCAAACCGG);
SEQ ID NO: 100
(TCATAGTTTCCAGCAAACCG);
SEQ ID NO: 101
(AGGTCATAGTTTCCAGCAAA);
SEQ ID NO: 102
(GGTAGACTAGGTCATAGTTT);
SEQ ID NO: 103
(AGGTAGACTAGGTCATAGTT);
SEQ ID NO: 104
(CAGGTAGACTAGGTCATAGT);
SEQ ID NO: 105
(CTTCACAGTGCAGGTAGACTAGGTC);
SEQ ID NO: 106
(ACAGTGCAGGTAGACTAGGT);
SEQ ID NO: 107
(CACAGTGCAGGTAGACTAGG);
SEQ ID NO: 108
(TCACAGTGCAGGTAGACTAG);
SEQ ID NO: 109
(TTCACAGTGCAGGTAGACTA);
SEQ ID NO: 110
(GTCACAGAGATAGACTTCAC);
SEQ ID NO: 111
(TGTCACAGAGATAGACTTCA);
SEQ ID NO: 112
(GTGTCACAGAGATAGACTTC);
SEQ ID NO: 113
(TCATTCATGGTGTCACAGAGATAGA);
SEQ ID NO: 114
(TTCATGGTGTCACAGAGATA);
SEQ ID NO: 115
(ATTCATGGTGTCACAGAGAT);
and
SEQ ID NO: 120
(GAGTCAACTCACAGGCTTGCACTTT).

In some embodiments, the targeting sequence is SEQ ID NO: 82. In some embodiments, the targeting sequence is SEQ ID NO: 84. In some embodiments, the targeting sequence is SEQ ID NO: 85. In some embodiments, the targeting sequence is SEQ ID NO: 86. In some embodiments, the targeting sequence is SEQ ID NO: 89. In some embodiments, the targeting sequence is SEQ ID NO: 90. In some embodiments, the targeting sequence is SEQ ID NO: 91. In some embodiments, the targeting sequence is SEQ ID NO: 92. In some embodiments, the targeting sequence is SEQ ID NO: 93. In some embodiments, the targeting sequence is SEQ ID NO: 94. In some embodiments, the targeting sequence is SEQ ID NO: 95. In some embodiments, the targeting sequence is SEQ ID NO: 96. In some embodiments, the targeting sequence is SEQ ID NO: 98. In some embodiments, the targeting sequence is SEQ ID NO: 99. In some embodiments, the targeting sequence is SEQ ID NO: 100. In some embodiments, the targeting sequence is SEQ ID NO: 101. In some embodiments, the targeting sequence is SEQ ID NO: 102. In some embodiments, the targeting sequence is SEQ ID NO: 103. In some embodiments, the targeting sequence is SEQ ID NO: 104. In some embodiments, the targeting sequence is SEQ ID NO: 105. In some embodiments, the targeting sequence is SEQ ID NO: 106. In some embodiments, the targeting sequence is SEQ ID NO: 107. In some embodiments, the targeting sequence is SEQ ID NO: 108. In some embodiments, the targeting sequence is SEQ ID NO: 109. In some embodiments, the targeting sequence is SEQ ID NO: 110. In some embodiments, the targeting sequence is SEQ ID NO: 111. In some embodiments, the targeting sequence is SEQ ID NO: 112. In some embodiments, the targeting sequence is SEQ ID NO: 113. In some embodiments, the targeting sequence is SEQ ID NO: 114. In some embodiments, the targeting sequence is SEQ ID NO: 115. In some embodiments, the targeting sequence is SEQ ID NO: 120.

In some embodiments, the target region is H8A (−2+23). In some embodiments, the target region is H8A(+51+75). In some embodiments, the target region is H8A(+60+79). In some embodiments, the target region is H8A(+61+80). In some embodiments, the target region is H8A(+68+87). In some embodiments, the target region is H8A(+69+88). In some embodiments, the target region is H8A(+70+89). In some embodiments, the target region is H8A(+76+95). In some embodiments, the target region is H8A(+76+100). In some embodiments, the target region is H8A(+77+96). In some embodiments, the target region is H8A(+78+97). In some embodiments, the target region is H8A(+79+98). In some embodiments, the target region is H8A(+81+100). In some embodiments, the target region is H8A(+82+101). In some embodiments, the target region is H8A(+83+102). In some embodiments, the target region is H8A(+86+105). In some embodiments, the target region is H8A(+94+113). In some embodiments, the target region is H8A(+95+114). In some embodiments, the target region is H8A(+96+115). In some embodiments, the target region is H8A(+101+125). In some embodiments, the target region is H8A(+102+121). In some embodiments, the target region is H8A(+103+122). In some embodiments, the target region is H8A(+104+123). In some embodiments, the target region is H8A(+105+124). In some embodiments, the target region is H8A(+120+139). In some embodiments, the target region is H8A(+121+140). In some embodiments, the target region is H8A(+122+141). In some embodiments, the target region is H8A(+126+150). In some embodiments, the target region is H8A(+128+147). In some embodiments, the target region is H8A(+129+148). In some embodiments, the target region is H8D (+12-13).

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to the target region H8A (−2+23). In a further embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising SEQ ID NO: 82. In still a further embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to the target region H8A (−2+23). In a further embodiment, antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising SEQ ID NO: 82.

In a further embodiment, the target region is selected from H8A(+51+75), H8A(+60+79), and H8A(+61+80). In certain embodiments, the antisense oligomer, or a pharmaceutically acceptable salt thereof, has a targeting sequence comprising a sequence selected from SEQ ID NOs: 84-86.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 68^{th} nucleotide to the 100^{th} nucleotide measured from the 5′ end of exon 8 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 172. In another embodiment, the target region is selected from H8A(+68+87), H8A(+69+88), H8A(+70+89), H8A(+76+95), H8A(+76+100), H8A(+77+96), H8A(+78+97), and H8A(+79+98). In yet another embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising a sequence selected from SEQ ID NOs: 89-96.

In a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 76^{th} nucleotide to the 100^{th} nucleotide measured from the 5′ end of exon 8, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 17. In an embodiment, the target region is selected from H8A(+76+95), H8A(+77+96), H8A(+78+97), and H8A(+79+98). In another embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising a sequence selected from SEQ ID NOs: 92 and 94-96.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 81^{st} nucleotide to the 105^{th} nucleotide measured from the 5′ end of exon 8 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 174. In a further embodiment, the target region is selected from H8A(+81+100), H8A(+82+101), H8A(+83+102), and H8A(+86+105). In still a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising a sequence selected from SEQ ID NOs: 98-101.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 94^{th} nucleotide to the 124^{th} nucleotide measured from the 5′ end of exon 8 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 175. In a further embodiment, the target region is selected from H8A(+94+113), H8A(+95+114), H8A(+96+115), H8A(+101+125), H8A(+102+121), H8A(+103+122), H8A(+104+123), and H8A(+105+124). In still a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising a sequence selected from SEQ ID NOs: 102-109.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 120^{th} nucleotide to the 148^{th} nucleotide measured from the 5′ end of exon 8 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 176. In a further embodiment, the target region is selected from H8A(+120+139), H8A(+121+140), H8A(+122+141), H8A(+126+150), H8A(+128+147), and H8A(+129+148). In still a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence comprising a sequence selected from SEQ ID NOs: 110-115.

In a further embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 126^{th} nucleotide to the 148^{th} nucleotide measured from the 5′ end of exon 8, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and Formula (IV), have a targeting sequence complementary to a target region comprised in SEQ ID NO: 177. In certain embodiments, the target region is selected from H8A(+126+150), H8A(+128+147), and H8A(+129+148). In a further embodiment, the antisense oligomer, or a pharmaceutically acceptable salt thereof, has a targeting sequence comprising a sequence selected from SEQ ID NOs: 113-115. In some embodiments, the target region is H8A(+128+147). In some embodiments, the antisense oligomer, or a pharmaceutically acceptable salt thereof, has a targeting sequence comprising SEQ ID NO: 114.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to the target region H8D (+12-13). In another embodiment, the antisense oligomer, or a pharmaceutically acceptable salt thereof, has a targeting sequence comprising SEQ ID NO: 120.

In still another embodiment of the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), the target region is an intron/exon junction or an exon internal region of exon 9. In certain embodiments, the target region of exon 9 is selected from H9A (−5+20), H9A(+1+25), H9A(+51+75), and H9D (+7-18). In some embodiments, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region of exon 9, wherein the targeting sequence comprises a sequence selected from:

SEQ ID NO: 121
(AATCTGGTCCCAGAGCAGGTCTACA);
SEQ ID NO: 122
(TTCGGAATCTGGTCCCAGAGCAGGT);
SEQ ID NO: 123
(TGTGATGGGACCCAAGTTCAGGACA);
and
SEQ ID NO: 124
(AGCGAGTGGCTCTCTTACCTTTCCG).

In some embodiments, the targeting sequence is SEQ ID NO 121. In some embodiments, the targeting sequence is SEQ ID NO: 122. In some embodiments, the targeting sequence is SEQ ID NO: 123. In some embodiments, the targeting sequence is SEQ ID NO: 124.

In some embodiments, the target region is H9A (−5+20). In some embodiments, the target region is H9A(+1+25). In some embodiments, the target region is H9A(+51+75). In some embodiments, the target region is H9D (+7-18).

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a target region within the 5^{th} nucleotide of intron 8 measured from the 5′ end of exon 9 and the 25^{th} nucleotide of exon 9 measured from the 5′ end of exon 9 of the human UMOD gene pre-mRNA. In a further embodiment, the target region Is selected from H9A (−5+20) and H9A(+1+25). In still a further embodiment, the antisense oligomer as a targeting sequence comprising a sequence selected from SEQ ID NOs: 121 and 122.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a target sequence complementary to the target region H9A(+51+75). In some embodiments, the antisense oligomer, or a pharmaceutically acceptable salt thereof, has a targeting sequence comprising SEQ ID NO: 123.

In another embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a target sequence complementary to the target region H9D (+7-18). In some embodiments, the antisense oligomer, or a pharmaceutically acceptable salt thereof, has a targeting sequence comprising SEQ ID NO: 124.

In an embodiment, the antisense oligomers, or pharmaceutically acceptable salts thereof, disclosed herein, including antisense oligomers, or pharmaceutically acceptable salts thereof, of Formula (I), Formula (IA), Formula (II), Formula (III), and/or Formula (IV), have a targeting sequence complementary to a region within an intron/exon junction or an exon internal junction of the human UMOD gene pre-mRNA, wherein the targeting sequence comprises a sequence selected from:

SEQ ID NO: 32
(GGGAGATATCTGAAACAGGT);
SEQ ID NO: 37
(AAGACCTTGTCGAAGCCCAGACTCT);
SEQ ID NO: 62
(TTTGATGTTGAGGTCACGGA);
SEQ ID NO: 70
(GCGGTCTTCAGGCTGACTTT);
SEQ ID NO: 74
(GGCTGTAGGGCGGTCTTCAG);
SEQ ID NO: 81
(CCACACCTGACCATTGGCTG);
SEQ ID NO: 114
(TTCATGGTGTCACAGAGATA);
and
SEQ ID NO: 120
(GAGTCAACTCACAGGCTTGCACTTT).

### Formulae (I) and (IA)

In certain embodiments, the antisense oligomer has structural Formula (I):

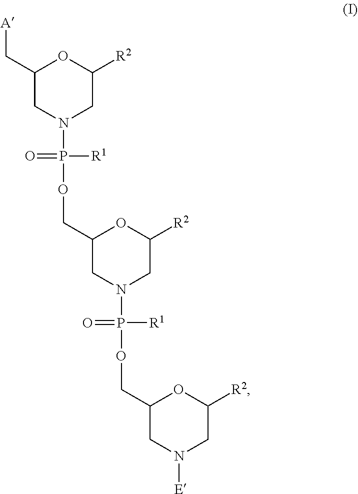

or a pharmaceutically acceptable salt thereof,
wherein:
A′ is selected from —OH,

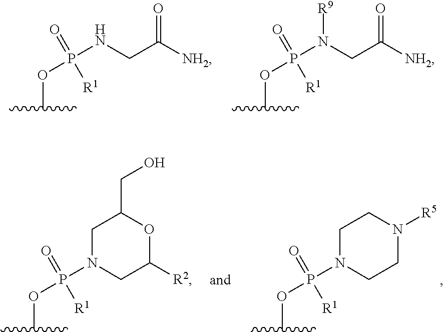

wherein:
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, —C₆₋₁₀-aryl-R^{6, 5-10} membered heteroaryl-R⁶, —C(O)O—(C₁₋₆-alkyl)-R⁶, —C(O)O-aryl-R⁶, —C(O)O-(5-10 membered heteroaryl)-R⁶, and

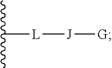

R⁶ is selected from —OH, —SH, and —NH₂, or R⁶ is O, S, or NH, each of which is covalently linked to a solid support;
R⁹ is C₁₋₆ alkyl;
each R¹ is independently selected from —OH and —N(R³)(R⁴), wherein each R³ and R⁴ is, independently at each occurrence, —H or —C₁₋₆-alkyl;
each R² is independently selected from a naturally or non-naturally occurring nucleobase, which, when taken together, forms a targeting sequence of 13 to 30 bases, that is complementary to a target region within a pre-mRNA of the human uromodulin (UMOD) gene (SEQ ID NO: 1) wherein the target region is an intron/exon junction or an exon internal region of the human UMOD gene pre-mRNA;
t is 11-28;
E′ is selected from —H, —C₁₋₆-alkyl, —C(O)C₁₋₆-alkyl, benzoyl, stearoyl, trityl, monomethoxytrityl, dimethoxytrityl, trimethoxytrityl,

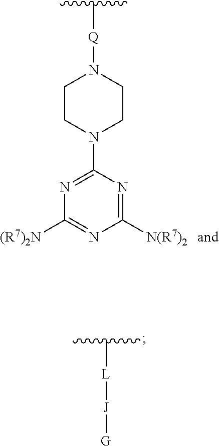

wherein:
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH) NH₂;
L is a linking amino acid, wherein L is covalently-linked by an amide bond to the C-terminus of J;
J is a cell-penetrating peptide; and
G is selected from —H, —C(O)C₁₋₆-alkyl, benzoyl, and stearoyl, wherein G is covalently-linked to J.

In some embodiments, t is 11, 12, 13, 14, 15, 16, 17, 18, 19, 20, 21, 22, 23, 24, 25, 26, 27, or 28.

In some embodiments, each R² is independently selected from a naturally or non-naturally occurring nucleobase, which, when taken together, forms a targeting sequence of from 13 to 30 bases in length, that is complementary to a target region within a pre-mRNA of the human uromodulin (UMOD) gene (SEQ ID NO: 1) wherein the target region is an intron/exon junction or an exon internal region of the human UMOD gene pre-mRNA.

In an embodiment, E′ is selected from H, —C₁₋₆-alkyl, —C(O)C₁₋₆-alkyl, benzoyl, stearoyl, trityl, monomethoxytrityl, dimethoxytrityl, trimethoxytrityl, and

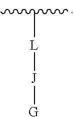

In a further embodiment, E′ is selected from —H, —C(O) CH₃, benzoyl, stearoyl, trityl, 4-methoxytrityl, and

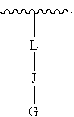

In an embodiment, A′ is selected from:

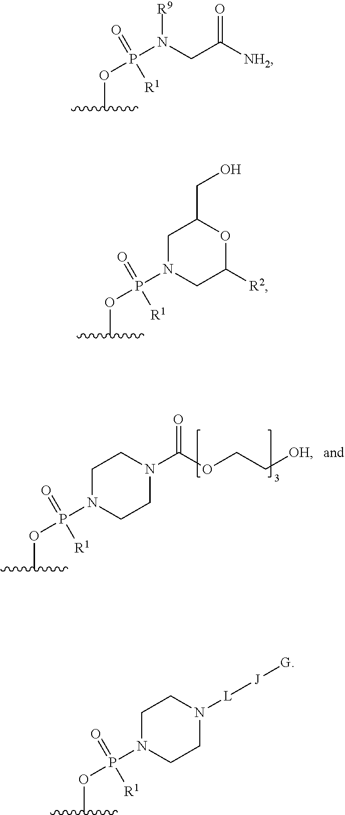

In an embodiment, at least one of the following is true:
A′ is

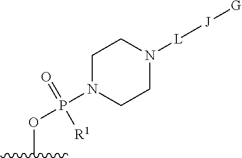

or (2) E′ is

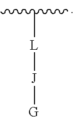

In an embodiment, A′ is selected from:

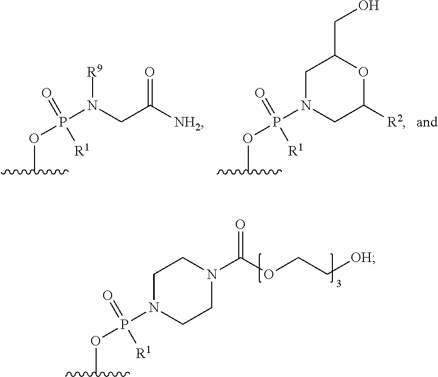

and E′ is

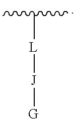

In an embodiment, A′ is

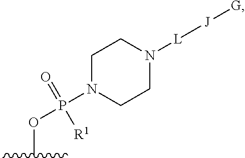

and
E′ is selected from H, —C(O) CH₃, trityl, 4-methoxytrityl, benzoyl, and stearoyl.

In an embodiment, each R¹ is-N(CH₃)₂. In an embodiment, L is glycine, proline, or β-alanine. In an embodiment, L is glycine. In an embodiment, L is proline.

In some embodiments, the antisense oligomer of Formula (I) is an antisense oligomer of Formula (IA):

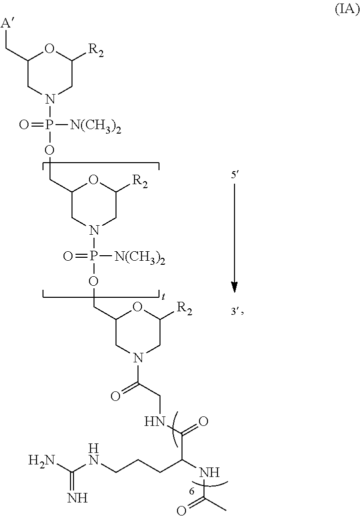

or a pharmaceutically acceptable salt thereof, wherein:
A′ is a moiety selected from:

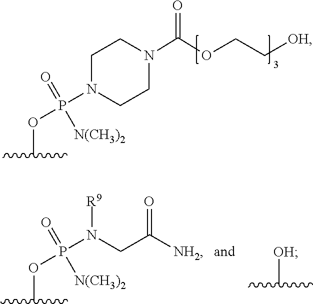

and
t is 11-28;
each R² is independently selected from a naturally or non-naturally occurring nucleobase, which, when taken together, forms a targeting sequence of 13 to 30 bases in length that is complementary to a target region within a pre-mRNA of the human uromodulin (UMOD) gene represented by SEQ ID NO: 1, wherein the target region is an intron/exon junction or an exon internal region of the human UMOD gene pre-mRNA;
R⁹ is C₁₋₆ alkyl.

In an embodiment of Formula (I) and/or (IA), t is an integer from 11 to 28. In an embodiment of Formula (I) and/or (IA), t is at least 11. In an embodiment of Formula (I) and/or (IA), t is up to 28. In an embodiment of Formula (I) and/or (IA), t is 11-28. In an embodiment of Formula (I) and/or (IA), t is 11-27. In an embodiment of Formula (I) and/or (IA), t is 11-26. In an embodiment of Formula (I) and/or (IA), t is 11-25. In an embodiment of Formula (I) and/or (IA), t is 11-24. In an embodiment of Formula (I) and/or (IA), t is 11-23. In an embodiment of Formula (I) and/or (IA), t is 11-22. In an embodiment of Formula (I) and/or (IA), t is 11-21. In an embodiment of Formula (I) and/or (IA), t is 11-20. In an embodiment of Formula (I) and/or (IA), t is 11-19. In an embodiment of Formula (I) and/or (IA), t is 11-18. In an embodiment of Formula (I) and/or (IA), t is 11-17. In an embodiment of Formula (I) and/or (IA), t is 11-16. In an embodiment of Formula (I) and/or (IA), t is 11-15. In an embodiment of Formula (I) and/or (IA), t is 11-14. In an embodiment of Formula (I) and/or (IA), t is 11-13. In an embodiment of Formula (I) and/or (IA), t is 11-12. In an embodiment of Formula (I) and/or (IA), t is 12-28. In an embodiment of Formula (I) and/or (IA), t is 12-27. In an embodiment of Formula (I) and/or (IA), t is 12-26. In an embodiment of Formula (I) and/or (IA), t is 12-25. In an embodiment of Formula (I) and/or (IA), t is 12-24. In an embodiment of Formula (I) and/or (IA), t is 12-23. In an embodiment of Formula (I) and/or (IA), t is 12-22. In an embodiment of Formula (I) and/or (IA), t is 12-21. In an embodiment of Formula (I) and/or (IA), t is 12-20. In an embodiment of Formula (I) and/or (IA), t is 12-19. In an embodiment of Formula (I) and/or (IA), t is 12-18. In an embodiment of Formula (I) and/or (IA), t is 12-17. In an embodiment of Formula (I) and/or (IA), t is 12-16. In an embodiment of Formula (I) and/or (IA), t is 12-15. In an embodiment of Formula (I) and/or (IA), t is 12-14. In an embodiment of Formula (I) and/or (IA), t is 12-13. In an embodiment of Formula (I) and/or (IA), t is 13-28. In an embodiment of Formula (I) and/or (IA), t is 13-27. In an embodiment of Formula (I) and/or (IA), t is 13-26. In an embodiment of Formula (I) and/or (IA), t is 13-25. In an embodiment of Formula (I) and/or (IA), t is 13-24. In an embodiment of Formula (I) and/or (IA), t is 13-23. In an embodiment of Formula (I) and/or (IA), t is 13-22. In an embodiment of Formula (I) and/or (IA), t is 13-21. In an embodiment of Formula (I) and/or (IA), t is 13-20. In an embodiment of Formula (I) and/or (IA), t is 13-19. In an embodiment of Formula (I) and/or (IA), t is 13-18. In an embodiment of Formula (I) and/or (IA), t is 13-17. In an embodiment of Formula (I) and/or (IA), t is 13-16. In an embodiment of Formula (I) and/or (IA), t is 13-15. In an embodiment of Formula (I) and/or (IA), t is 13-14. In an embodiment of Formula (I) and/or (IA), t is 14-28. In an embodiment of Formula (I) and/or (IA), t is 14-27. In an embodiment of Formula (I) and/or (IA), t is 14-26. In an embodiment of Formula (I) and/or (IA), t is 14-25. In an embodiment of Formula (I) and/or (IA), t is 14-24. In an embodiment of Formula (I) and/or (IA), t is 14-23. In an embodiment of Formula (I) and/or (IA), t is 14-22. In an embodiment of Formula (I) and/or (IA), t is 14-21. In an embodiment of Formula (I) and/or (IA), t is 14-20. In an embodiment of Formula (I) and/or (IA), t is 14-19. In an embodiment of Formula (I) and/or (IA), t is 14-18. In an embodiment of Formula (I) and/or (IA), t is 14-17. In an embodiment of Formula (I) and/or (IA), t is 14-16. In an embodiment of Formula (I) and/or (IA), t is 14-15. In an embodiment of Formula (I) and/or (IA), t is 15-28. In an embodiment of Formula (I) and/or (IA), t is 15-27. In an embodiment of Formula (I) and/or (IA), t is 15-26. In an embodiment of Formula (I) and/or (IA), t is 15-25. In an embodiment of Formula (I) and/or (IA), t is 15-24. In an embodiment of Formula (I) and/or (IA), t is 15-23. In an embodiment of Formula (I) and/or (IA), t is 15-22. In an embodiment of Formula (I) and/or (IA), t is 15-21. In an embodiment of Formula (I) and/or (IA), t is 15-20. In an embodiment of Formula (I) and/or (IA), t is 15-19. In an embodiment of Formula (I) and/or (IA), t is 15-18. In an embodiment of Formula (I) and/or (IA), t is 15-17. In an embodiment of Formula (I) and/or (IA), t is 15-16. In an embodiment of Formula (I) and/or (IA), t is 16-28. In an embodiment of Formula (I) and/or (IA), t is 16-27. In an embodiment of Formula (I) and/or (IA), t is 16-26. In an embodiment of Formula (I) and/or (IA), t is 16-25. In an embodiment of Formula (I) and/or (IA), t is 16-24. In an embodiment of Formula (I) and/or (IA), t is 16-23. In an embodiment of Formula (I) and/or (IA), t is 16-22. In an embodiment of Formula (I) and/or (IA), t is 16-21. In an embodiment of Formula (I) and/or (IA), t is 16-20. In an embodiment of Formula (I) and/or (IA), t is 16-19. In an embodiment of Formula (I) and/or (IA), t is 16-18. In an embodiment of Formula (I) and/or (IA), t is 16-17. In an embodiment of Formula (I) and/or (IA), t is 17-28. In an embodiment of Formula (I) and/or (IA), t is 17-27. In an embodiment of Formula (I) and/or (IA), t is 17-26. In an embodiment of Formula (I) and/or (IA), t is 17-25. In an embodiment of Formula (I) and/or (IA), t is 17-24. In an embodiment of Formula (I) and/or (IA), t is 17-23. In an embodiment of Formula (I) and/or (IA), t is 17-22. In an embodiment of Formula (I) and/or (IA), t is 17-21. In an embodiment of Formula (I) and/or (IA), t is 17-20. In an embodiment of Formula (I) and/or (IA), t is 17-19. In an embodiment of Formula (I) and/or (IA), t is 17-18. In an embodiment of Formula (I) and/or (IA), t is 18-28. In an embodiment of Formula (I) and/or (IA), t is 18-27. In an embodiment of Formula (I) and/or (IA), t is 18-26. In an embodiment of Formula (I) and/or (IA), t is 18-25. In an embodiment of Formula (I) and/or (IA), t is 18-24. In an embodiment of Formula (I) and/or (IA), t is 18-23. In an embodiment of Formula (I) and/or (IA), t is 18-22. In an embodiment of Formula (I) and/or (IA), t is 18-21. In an embodiment of Formula (I) and/or (IA), t is 18-20. In an embodiment of Formula (I) and/or (IA), t is 18-19. In an embodiment of Formula (I) and/or (IA), t is 19-28. In an embodiment of Formula (I) and/or (IA), t is 19-27. In an embodiment of Formula (I) and/or (IA), t is 19-26. In an embodiment of Formula (I) and/or (IA), t is 19-25. In an embodiment of Formula (I) and/or (IA), t is 19-24. In an embodiment of Formula (I) and/or (IA), t is 19-23. In an embodiment of Formula (I) and/or (IA), t is 19-22. In an embodiment of Formula (I) and/or (IA), t is 19-21. In an embodiment of Formula (I) and/or (IA), t is 19-20. In an embodiment of Formula (I) and/or (IA), t is 20-28. In an embodiment of Formula (I) and/or (IA), t is 20-27. In an embodiment of Formula (I) and/or (IA), t is 20-26. In an embodiment of Formula (I) and/or (IA), t is 20-25. In an embodiment of Formula (I) and/or (IA), t is 20-24. In an embodiment of Formula (I) and/or (IA), t is 20-23. In an embodiment of Formula (I) and/or (IA), t is 20-22. In an embodiment of Formula (I) and/or (IA), t is 20-21. In an embodiment of Formula (I) and/or (IA), t is 21-28. In an embodiment of Formula (I) and/or (IA), t is 21-27. In an embodiment of Formula (I) and/or (IA), t is 21-26. In an embodiment of Formula (I) and/or (IA), t is 21-25. In an embodiment of Formula (I) and/or (IA), t is 21-24. In an embodiment of Formula (I) and/or (IA), t is 21-23. In an embodiment of Formula (I) and/or (IA), t is 21-22. In an embodiment of Formula (I) and/or (IA), t is 22-28. In an embodiment of Formula (I) and/or (IA), t is 22-27. In an embodiment of Formula (I) and/or (IA), t is 22-26. In an embodiment of Formula (I) and/or (IA), t is 22-25. In an embodiment of Formula (I) and/or (IA), t is 22-24. In an embodiment of Formula (I) and/or (IA), t is 22-23. In an embodiment of Formula (I) and/or (IA), t is 23-28. In an embodiment of Formula (I) and/or (IA), t is 23-27. In an embodiment of Formula (I) and/or (IA), t is 23-26. In an embodiment of Formula (I) and/or (IA), t is 23-25. In an embodiment of Formula (I) and/or (IA), t is 23-24. In an embodiment of Formula (I) and/or (IA), t is 24-28. In an embodiment of Formula (I) and/or (IA), t is 24-27. In an embodiment of Formula (I) and/or (IA), t is 24-26. In an embodiment of Formula (I) and/or (IA), t is 24-25. In an embodiment of Formula (I) and/or (IA), t is 25-28. In an embodiment of Formula (I) and/or (IA), t is 25-27. In an embodiment of Formula (I) and/or (IA), t is 25-26. In another embodiment of Formula (I) and/or (IA), t is 26-30. In an embodiment of Formula (I) and/or (IA), t is 26-29. In an embodiment of Formula (I) and/or (IA), t is 26-28. In an embodiment of Formula (I) and/or (IA), t is 26-27. In an embodiment of Formula (I) and/or (IA), t is 27-28.

In an embodiment of Formula (I) and/or (IA), t is 11. In an embodiment of Formula (I) and/or (IA), t is 12. In an embodiment of Formula (I) and/or (IA), t is 13. In an embodiment of Formula (I) and/or (IA), t is 14. In an embodiment of Formula (I) and/or (IA), t is 15. In an embodiment of Formula (I) and/or (IA), t is 16. In an embodiment of Formula (I) and/or (IA), t is 17. In an embodiment of Formula (I) and/or (IA), t is 18. In an embodiment of Formula (I) and/or (IA), t is 19. In an embodiment of Formula (I) and/or (IA), t is 20. In an embodiment of Formula (I) and/or (IA), t is 21. In an embodiment of Formula (I) and/or (IA), t is 22. In an embodiment of Formula (I) and/or (IA), t is 23. In an embodiment of Formula (I) and/or (IA), t is 24. In an embodiment of Formula (I) and/or (IA), t is 25. In an embodiment of Formula (I) and/or (IA), t is 26. In an embodiment of Formula (I) and/or (IA), t is 27. In an embodiment of Formula (I) and/or (IA), t is 28.

In an embodiment of Formula (I) and/or (IA), each R² is independently selected from a naturally or non-naturally occurring nucleobase, which, when taken together, forms a targeting sequence of from 13 to 30 bases in length. In an embodiment of Formula (I) and/or (IA), each R² is independently selected from a naturally or non-naturally occurring nucleobase, which, when taken together, forms a targeting sequence at least 13 bases in length. In an embodiment of Formula (I) and/or (IA), each R² is independently selected from a naturally or non-naturally occurring nucleobase, which, when taken together, forms a targeting sequence of up to 30 bases in length.

In an embodiment of Formula (I) and/or (IA), each R² is independently selected from a naturally or non-naturally occurring nucleobase, which, when taken together, forms a targeting sequence of 13-29 bases in length. In an embodiment of Formula (I) and/or (IA), each R² is independently selected from a naturally or non-naturally occurring nucleobase, which, when taken together, forms a targeting sequence of 13-28 bases in length. In an embodiment of Formula (I) and/or (IA), each R² is independently selected from a naturally or non-naturally occurring nucleobase, which, when taken together, forms a targeting sequence of 13-27 bases in length. In an embodiment of Formula (I) and/or (IA), each R² is independently selected from a naturally or non-naturally occurring nucleobase, which, when taken together, forms a targeting sequence of 13-26 bases in length. In an embodiment of Formula (I) and/or (IA), each R² is independently selected from a naturally or non-naturally occurring nucleobase, which, when taken together, forms a targeting sequence of 13-25 bases in length. In an embodiment of Formula (I) and/or (IA), each R² is independently selected from a naturally or non-naturally occurring nucleobase, which, when taken together, forms a targeting sequence of 13-24 bases in length. In an embodiment of Formula (I) and/or (IA), each R² is independently selected from a naturally or non-naturally occurring nucleobase, which, when taken together, forms a targeting sequence of 13-23 bases in length. In an embodiment of Formula (I) and/or (IA), each R² is independently selected from a naturally or non-naturally occurring nucleobase, which, when taken together, forms a targeting sequence of 13-22 bases in length. In an embodiment of Formula (I) and/or (IA), each R² is independently selected from a naturally or non-naturally occurring nucleobase, which, when taken together, forms a targeting sequence of 13-21 bases in length. In an embodiment of Formula (I) and/or (IA), each R² is independently selected from a naturally or non-naturally occurring nucleobase, which, when taken together, forms a targeting sequence of 13-20 bases in length. In an embodiment of Formula (I) and/or (IA), each R² is independently selected from a naturally or non-naturally occurring nucleobase, which, when taken together, forms a targeting sequence of 13-19 bases in length. In an embodiment of Formula (I) and/or (IA), each R² is independently selected from a naturally or non-naturally occurring nucleobase, which, when taken together, forms a targeting sequence of 13-18 bases in length. In an embodiment of Formula (I) and/or (IA), each R² is independently selected from a naturally or non-naturally occurring nucleobase, which, when taken together, forms a targeting sequence of 13-17 bases in length. In an embodiment of Formula (I) and/or (IA), each R² is independently selected from a naturally or non-naturally occurring nucleobase, which, when taken together, forms a targeting sequence of 13-16 bases in length. In an embodiment of Formula (I) and/or (IA), each R² is independently selected from a naturally or non-naturally occurring nucleobase, which, when taken together, forms a targeting sequence of 13-15 bases in length. In an embodiment of Formula (I) and/or (IA), each R² is independently selected from a naturally or non-naturally occurring nucleobase, which, when taken together, forms a targeting sequence of 13-14 bases in length.

In another embodiment of Formula (I) and/or (IA), each R² is independently selected from a naturally or non-naturally occurring nucleobase, which, when taken together, forms a targeting sequence of 14-30 bases in length. In an embodiment of Formula (I) and/or (IA), each R² is independently selected from a naturally or non-naturally occurring nucleobase, which, when taken together, forms a targeting sequence of 14-29 bases in length. In an embodiment of Formula (I) and/or (IA), each R² is independently selected from a naturally or non-naturally occurring nucleobase, which, when taken together, forms a targeting sequence of 14-28 bases in length. In an embodiment of Formula (I) and/or (IA), each R² is independently selected from a naturally or non-naturally occurring nucleobase, which, when taken together, forms a targeting sequence of 14-27 bases in length. In an embodiment of Formula (I) and/or (IA), each R² is independently selected from a naturally or non-naturally occurring nucleobase, which, when taken together, forms a targeting sequence of 14-26 bases in length. In an embodiment of Formula (I) and/or (IA), each R² is independently selected from a naturally or non-naturally occurring nucleobase, which, when taken together, forms a targeting sequence of 14-25 bases in length. In an embodiment of Formula (I) and/or (IA), each R² is independently selected from a naturally or non-naturally occurring nucleobase, which, when taken together, forms a targeting sequence of 14-24 bases in length. In an embodiment of Formula (I) and/or (IA), each R² is independently selected from a naturally or non-naturally occurring nucleobase, which, when taken together, forms a targeting sequence of 14-23 bases in length. In an embodiment of Formula (I) and/or (IA), each R² is independently selected from a naturally or non-naturally occurring nucleobase, which, when taken together, forms a targeting sequence of 14-22 bases in length. In an embodiment of Formula (I) and/or (IA), each R² is independently selected from a naturally or non-naturally occurring nucleobase, which, when taken together, forms a targeting sequence of 14-21 bases in length. In an embodiment of Formula (I) and/or (IA), each R² is independently selected from a naturally or non-naturally occurring nucleobase, which, when taken together, forms a targeting sequence of 14-20 bases in length. In an embodiment of Formula (I) and/or (IA), each R² is independently selected from a naturally or non-naturally occurring nucleobase, which, when taken together, forms a targeting sequence of 14-19 bases in length. In an embodiment of Formula (I) and/or (IA), each R² is independently selected from a naturally or non-naturally occurring nucleobase, which, when taken together, forms a targeting sequence of 14-18 bases in length. In an embodiment of Formula (I) and/or (IA), each R² is independently selected from a naturally or non-naturally occurring nucleobase, which, when taken together, forms a targeting sequence of 14-17 bases in length. In an embodiment of Formula (I) and/or (IA), each R² is independently selected from a naturally or non-naturally occurring nucleobase, which, when taken together, forms a targeting sequence of 14-16 bases in length. In an embodiment of Formula (I) and/or (IA), each R² is independently selected from a naturally or non-naturally occurring nucleobase, which, when taken together, forms a targeting sequence of 14-15 bases in length.

In another embodiment of Formula (I) and/or (IA), each R² is independently selected from a naturally or non-naturally occurring nucleobase, which, when taken together, forms a targeting sequence of 15-30 bases in length. In an embodiment of Formula (I) and/or (IA), each R² is independently selected from a naturally or non-naturally occurring nucleobase, which, when taken together, forms a targeting sequence of 15-29 bases in length. In an embodiment of Formula (I) and/or (IA), each R² is independently selected from a naturally or non-naturally occurring nucleobase, which, when taken together, forms a targeting sequence of 15-28 bases in length. In an embodiment of Formula (I) and/or (IA), each R² is independently selected from a naturally or non-naturally occurring nucleobase, which, when taken together, forms a targeting sequence of 15-27 bases in length. In an embodiment of Formula (I) and/or (IA), each R² is independently selected from a naturally or non-naturally occurring nucleobase, which, when taken together, forms a targeting sequence of 15-26 bases in length. In an embodiment of Formula (I) and/or (IA), each R² is independently selected from a naturally or non-naturally occurring nucleobase, which, when taken together, forms a targeting sequence of 15-25 bases in length. In an embodiment of Formula (I) and/or (IA), each R² is independently selected from a naturally or non-naturally occurring nucleobase, which, when taken together, forms a targeting sequence of 15-24 bases in length. In an embodiment of Formula (I) and/or (IA), each R² is independently selected from a naturally or non-naturally occurring nucleobase, which, when taken together, forms a targeting sequence of 15-23 bases in length. In an embodiment of Formula (I) and/or (IA), each R² is independently selected from a naturally or non-naturally occurring nucleobase, which, when taken together, forms a targeting sequence of 15-22 bases in length. In an embodiment of Formula (I) and/or (IA), each R² is independently selected from a naturally or non-naturally occurring nucleobase, which, when taken together, forms a targeting sequence of 15-21 bases in length. In an embodiment of Formula (I) and/or (IA), each R² is independently selected from a naturally or non-naturally occurring nucleobase, which, when taken together, forms a targeting sequence of 15-20 bases in length. In an embodiment of Formula (I) and/or (IA), each R² is independently selected from a naturally or non-naturally occurring nucleobase, which, when taken together, forms a targeting sequence of 15-19 bases in length. In an embodiment of Formula (I) and/or (IA), each R² is independently selected from a naturally or non-naturally occurring nucleobase, which, when taken together, forms a targeting sequence of 15-18 bases in length. In an embodiment of Formula (I) and/or (IA), each R² is independently selected from a naturally or non-naturally occurring nucleobase, which, when taken together, forms a targeting sequence of 15-17 bases in length. In an embodiment of Formula (I) and/or (IA), each R² is independently selected from a naturally or non-naturally occurring nucleobase, which, when taken together, forms a targeting sequence of 15-16 bases in length.

In another embodiment of Formula (I) and/or (IA), each R² is independently selected from a naturally or non-naturally occurring nucleobase, which, when taken together, forms a targeting sequence of 16-30 bases in length. In an embodiment of Formula (I) and/or (IA), each R² is independently selected from a naturally or non-naturally occurring nucleobase, which, when taken together, forms a targeting sequence of 16-29 bases in length. In an embodiment of Formula (I) and/or (IA), each R² is independently selected from a naturally or non-naturally occurring nucleobase, which, when taken together, forms a targeting sequence of 16-28 bases in length. In an embodiment of Formula (I) and/or (IA), each R² is independently selected from a naturally or non-naturally occurring nucleobase, which, when taken together, forms a targeting sequence of 16-27 bases in length. In an embodiment of Formula (I) and/or (IA), each R² is independently selected from a naturally or non-naturally occurring nucleobase, which, when taken together, forms a targeting sequence of 16-26 bases in length. In an embodiment of Formula (I) and/or (IA), each R² is independently selected from a naturally or non-naturally occurring nucleobase, which, when taken together, forms a targeting sequence of 16-25 bases in length. In an embodiment of Formula (I) and/or (IA), each R² is independently selected from a naturally or non-naturally occurring nucleobase, which, when taken together, forms a targeting sequence of 16-24 bases in length. In an embodiment of Formula (I) and/or (IA), each R² is independently selected from a naturally or non-naturally occurring nucleobase, which, when taken together, forms a targeting sequence of 16-23 bases in length. In an embodiment of Formula (I) and/or (IA), each R² is independently selected from a naturally or non-naturally occurring nucleobase, which, when taken together, forms a targeting sequence of 16-22 bases in length. In an embodiment of Formula (I) and/or (IA), each R² is independently selected from a naturally or non-naturally occurring nucleobase, which, when taken together, forms a targeting sequence of 16-21 bases in length. In an embodiment of Formula (I) and/or (IA), each R² is independently selected from a naturally or non-naturally occurring nucleobase, which, when taken together, forms a targeting sequence of 16-20 bases in length. In an embodiment of Formula (I) and/or (IA), each R² is independently selected from a naturally or non-naturally occurring nucleobase, which, when taken together, forms a targeting sequence of 16-19 bases in length. In an embodiment of Formula (I) and/or (IA), each R² is independently selected from a naturally or non-naturally occurring nucleobase, which, when taken together, forms a targeting sequence of 16-18 bases in length. In an embodiment of Formula (I) and/or (IA), each R² is independently selected from a naturally or non-naturally occurring nucleobase, which, when taken together, forms a targeting sequence of 16-17 bases in length.

In another embodiment of Formula (I) and/or (IA), each R² is independently selected from a naturally or non-naturally occurring nucleobase, which, when taken together, forms a targeting sequence of 17-30 bases in length. In an embodiment of Formula (I) and/or (IA), each R² is independently selected from a naturally or non-naturally occurring nucleobase, which, when taken together, forms a targeting sequence of 17-29 bases in length. In an embodiment of Formula (I) and/or (IA), each R² is independently selected from a naturally or non-naturally occurring nucleobase, which, when taken together, forms a targeting sequence of 17-28 bases in length. In an embodiment of Formula (I) and/or (IA), each R² is independently selected from a naturally or non-naturally occurring nucleobase, which, when taken together, forms a targeting sequence of 17-27 bases in length. In an embodiment of Formula (I) and/or (IA), each R² is independently selected from a naturally or non-naturally occurring nucleobase, which, when taken together, forms a targeting sequence of 17-26 bases in length. In an embodiment of Formula (I) and/or (IA), each R² is independently selected from a naturally or non-naturally occurring nucleobase, which, when taken together, forms a targeting sequence of 17-25 bases in length. In an embodiment of Formula (I) and/or (IA), each R² is independently selected from a naturally or non-naturally occurring nucleobase, which, when taken together, forms a targeting sequence of 17-24 bases in length. In an embodiment of Formula (I) and/or (IA), each R² is independently selected from a naturally or non-naturally occurring nucleobase, which, when taken together, forms a targeting sequence of 17-23 bases in length. In an embodiment of Formula (I) and/or (IA), each R² is independently selected from a naturally or non-naturally occurring nucleobase, which, when taken together, forms a targeting sequence of 17-22 bases in length. In an embodiment of Formula (I) and/or (IA), each R² is independently selected from a naturally or non-naturally occurring nucleobase, which, when taken together, forms a targeting sequence of 17-21 bases in length. In an embodiment of Formula (I) and/or (IA), each R² is independently selected from a naturally or non-naturally occurring nucleobase, which, when taken together, forms a targeting sequence of 17-20 bases in length. In an embodiment of Formula (I) and/or (IA), each R² is independently selected from a naturally or non-naturally occurring nucleobase, which, when taken together, forms a targeting sequence of 17-19 bases in length. In an embodiment of Formula (I) and/or (IA), each R² is independently selected from a naturally or non-naturally occurring nucleobase, which, when taken together, forms a targeting sequence of 17-18 bases in length.

In another embodiment of Formula (I) and/or (IA), each R² is independently selected from a naturally or non-naturally occurring nucleobase, which, when taken together, forms a targeting sequence of 18-30 bases in length. In an embodiment of Formula (I) and/or (IA), each R² is independently selected from a naturally or non-naturally occurring nucleobase, which, when taken together, forms a targeting sequence of 18-29 bases in length. In an embodiment of Formula (I) and/or (IA), each R² is independently selected from a naturally or non-naturally occurring nucleobase, which, when taken together, forms a targeting sequence of 18-28 bases in length. In an embodiment of Formula (I) and/or (IA), each R² is independently selected from a naturally or non-naturally occurring nucleobase, which, when taken together, forms a targeting sequence of 18-27 bases in length. In an embodiment of Formula (I) and/or (IA), each R² is independently selected from a naturally or non-naturally occurring nucleobase, which, when taken together, forms a targeting sequence of 18-26 bases in length. In an embodiment of Formula (I) and/or (IA), each R² is independently selected from a naturally or non-naturally occurring nucleobase, which, when taken together, forms a targeting sequence of 18-25 bases in length. In an embodiment of Formula (I) and/or (IA), each R² is independently selected from a naturally or non-naturally occurring nucleobase, which, when taken together, forms a targeting sequence of 18-24 bases in length. In an embodiment of Formula (I) and/or (IA), each R² is independently selected from a naturally or non-naturally occurring nucleobase, which, when taken together, forms a targeting sequence of 18-23 bases in length. In an embodiment of Formula (I) and/or (IA), each R² is independently selected from a naturally or non-naturally occurring nucleobase, which, when taken together, forms a targeting sequence of 18-22 bases in length. In an embodiment of Formula (I) and/or (IA), each R² is independently selected from a naturally or non-naturally occurring nucleobase, which, when taken together, forms a targeting sequence of 18-21 bases in length. In an embodiment of Formula (I) and/or (IA), each R² is independently selected from a naturally or non-naturally occurring nucleobase, which, when taken together, forms a targeting sequence of 18-20 bases in length. In an embodiment of Formula (I) and/or (IA), each R² is independently selected from a naturally or non-naturally occurring nucleobase, which, when taken together, forms a targeting sequence of 18-19 bases in length.

In another embodiment of Formula (I) and/or (IA), each R² is independently selected from a naturally or non-naturally occurring nucleobase, which, when taken together, forms a targeting sequence of 19-30 bases in length. In an embodiment of Formula (I) and/or (IA), each R² is independently selected from a naturally or non-naturally occurring nucleobase, which, when taken together, forms a targeting sequence of 19-29 bases in length. In an embodiment of Formula (I) and/or (IA), each R² is independently selected from a naturally or non-naturally occurring nucleobase, which, when taken together, forms a targeting sequence of 19-28 bases in length. In an embodiment of Formula (I) and/or (IA), each R² is independently selected from a naturally or non-naturally occurring nucleobase, which, when taken together, forms a targeting sequence of 19-27 bases in length. In an embodiment of Formula (I) and/or (IA), each R² is independently selected from a naturally or non-naturally occurring nucleobase, which, when taken together, forms a targeting sequence of 19-26 bases in length. In an embodiment of Formula (I) and/or (IA), each R² is independently selected from a naturally or non-naturally occurring nucleobase, which, when taken together, forms a targeting sequence of 19-25 bases in length. In an embodiment of Formula (I) and/or (IA), each R² is independently selected from a naturally or non-naturally occurring nucleobase, which, when taken together, forms a targeting sequence of 19-24 bases in length. In an embodiment of Formula (I) and/or (IA), each R² is independently selected from a naturally or non-naturally occurring nucleobase, which, when taken together, forms a targeting sequence of 19-23 bases in length. In an embodiment of Formula (I) and/or (IA), each R² is independently selected from a naturally or non-naturally occurring nucleobase, which, when taken together, forms a targeting sequence of 19-22 bases in length. In an embodiment of Formula (I) and/or (IA), each R² is independently selected from a naturally or non-naturally occurring nucleobase, which, when taken together, forms a targeting sequence of 19-21 bases in length. In an embodiment of Formula (I) and/or (IA), each R² is independently selected from a naturally or non-naturally occurring nucleobase, which, when taken together, forms a targeting sequence of 19-20 bases in length.

In another embodiment of Formula (I) and/or (IA), each R² is independently selected from a naturally or non-naturally occurring nucleobase, which, when taken together, forms a targeting sequence of 20-30 bases in length. In an embodiment of Formula (I) and/or (IA), each R² is independently selected from a naturally or non-naturally occurring nucleobase, which, when taken together, forms a targeting sequence of 20-29 bases in length. In an embodiment of Formula (I) and/or (IA), each R² is independently selected from a naturally or non-naturally occurring nucleobase, which, when taken together, forms a targeting sequence of 20-28 bases in length. In an embodiment of Formula (I) and/or (IA), each R² is independently selected from a naturally or non-naturally occurring nucleobase, which, when taken together, forms a targeting sequence of 20-27 bases in length. In an embodiment of Formula (I) and/or (IA), each R² is independently selected from a naturally or non-naturally occurring nucleobase, which, when taken together, forms a targeting sequence of 20-26 bases in length. In an embodiment of Formula (I) and/or (IA), each R² is independently selected from a naturally or non-naturally occurring nucleobase, which, when taken together, forms a targeting sequence of 20-25 bases in length. In an embodiment of Formula (I) and/or (IA), each R² is independently selected from a naturally or non-naturally occurring nucleobase, which, when taken together, forms a targeting sequence of 20-24 bases in length. In an embodiment of Formula (I) and/or (IA), each R² is independently selected from a naturally or non-naturally occurring nucleobase, which, when taken together, forms a targeting sequence of 20-23 bases in length. In an embodiment of Formula (I) and/or (IA), each R² is independently selected from a naturally or non-naturally occurring nucleobase, which, when taken together, forms a targeting sequence of 20-22 bases in length. In an embodiment of Formula (I) and/or (IA), each R² is independently selected from a naturally or non-naturally occurring nucleobase, which, when taken together, forms a targeting sequence of 20-21 bases in length.

In another embodiment of Formula (I) and/or (IA), each R² is independently selected from a naturally or non-naturally occurring nucleobase, which, when taken together, forms a targeting sequence of 21-30 bases in length. In an embodiment of Formula (I) and/or (IA), each R² is independently selected from a naturally or non-naturally occurring nucleobase, which, when taken together, forms a targeting sequence of 21-29 bases in length. In an embodiment of Formula (I) and/or (IA), each R² is independently selected from a naturally or non-naturally occurring nucleobase, which, when taken together, forms a targeting sequence of 21-28 bases in length. In an embodiment of Formula (I) and/or (IA), each R² is independently selected from a naturally or non-naturally occurring nucleobase, which, when taken together, forms a targeting sequence of 21-27 bases in length. In an embodiment of Formula (I) and/or (IA), each R² is independently selected from a naturally or non-naturally occurring nucleobase, which, when taken together, forms a targeting sequence of 21-26 bases in length. In an embodiment of Formula (I) and/or (IA), each R² is independently selected from a naturally or non-naturally occurring nucleobase, which, when taken together, forms a targeting sequence of 21-25 bases in length. In an embodiment of Formula (I) and/or (IA), each R² is independently selected from a naturally or non-naturally occurring nucleobase, which, when taken together, forms a targeting sequence of 21-24 bases in length. In an embodiment of Formula (I) and/or (IA), each R² is independently selected from a naturally or non-naturally occurring nucleobase, which, when taken together, forms a targeting sequence of 21-23 bases in length. In an embodiment of Formula (I) and/or (IA), each R² is independently selected from a naturally or non-naturally occurring nucleobase, which, when taken together, forms a targeting sequence of 21-22 bases in length.

In another embodiment of Formula (I) and/or (IA), each R² is independently selected from a naturally or non-naturally occurring nucleobase, which, when taken together, forms a targeting sequence of 22-30 bases in length. In an embodiment of Formula (I) and/or (IA), each R² is independently selected from a naturally or non-naturally occurring nucleobase, which, when taken together, forms a targeting sequence of 22-29 bases in length. In an embodiment of Formula (I) and/or (IA), each R² is independently selected from a naturally or non-naturally occurring nucleobase, which, when taken together, forms a targeting sequence of 22-28 bases in length. In an embodiment of Formula (I) and/or (IA), each R² is independently selected from a naturally or non-naturally occurring nucleobase, which, when taken together, forms a targeting sequence of 22-27 bases in length. In an embodiment of Formula (I) and/or (IA), each R² is independently selected from a naturally or non-naturally occurring nucleobase, which, when taken together, forms a targeting sequence of 22-26 bases in length. In an embodiment of Formula (I) and/or (IA), each R² is independently selected from a naturally or non-naturally occurring nucleobase, which, when taken together, forms a targeting sequence of 22-25 bases in length. In an embodiment of Formula (I) and/or (IA), each R² is independently selected from a naturally or non-naturally occurring nucleobase, which, when taken together, forms a targeting sequence of 22-24 bases in length. In an embodiment of Formula (I) and/or (IA), each R² is independently selected from a naturally or non-naturally occurring nucleobase, which, when taken together, forms a targeting sequence of 22-23 bases in length.

In another embodiment of Formula (I) and/or (IA), each R² is independently selected from a naturally or non-naturally occurring nucleobase, which, when taken together, forms a targeting sequence of 23-30 bases in length. In an embodiment of Formula (I) and/or (IA), each R² is independently selected from a naturally or non-naturally occurring nucleobase, which, when taken together, forms a targeting sequence of 23-29 bases in length. In an embodiment of Formula (I) and/or (IA), each R² is independently selected from a naturally or non-naturally occurring nucleobase, which, when taken together, forms a targeting sequence of 23-28 bases in length. In an embodiment of Formula (I) and/or (IA), each R² is independently selected from a naturally or non-naturally occurring nucleobase, which, when taken together, forms a targeting sequence of 23-27 bases in length. In an embodiment of Formula (I) and/or (IA), each R² is independently selected from a naturally or non-naturally occurring nucleobase, which, when taken together, forms a targeting sequence of 23-26 bases in length. In an embodiment of Formula (I) and/or (IA), each R² is independently selected from a naturally or non-naturally occurring nucleobase, which, when taken together, forms a targeting sequence of 23-25 bases in length. In an embodiment of Formula (I) and/or (IA), each R² is independently selected from a naturally or non-naturally occurring nucleobase, which, when taken together, forms a targeting sequence of 23-24 bases in length.

In another embodiment of Formula (I) and/or (IA), each R² is independently selected from a naturally or non-naturally occurring nucleobase, which, when taken together, forms a targeting sequence of 24-30 bases in length. In an embodiment of Formula (I) and/or (IA), each R² is independently selected from a naturally or non-naturally occurring nucleobase, which, when taken together, forms a targeting sequence of 24-29 bases in length. In an embodiment of Formula (I) and/or (IA), each R² is independently selected from a naturally or non-naturally occurring nucleobase, which, when taken together, forms a targeting sequence of 24-28 bases in length. In an embodiment of Formula (I) and/or (IA), each R² is independently selected from a naturally or non-naturally occurring nucleobase, which, when taken together, forms a targeting sequence of 24-27 bases in length. In an embodiment of Formula (I) and/or (IA), each R² is independently selected from a naturally or non-naturally occurring nucleobase, which, when taken together, forms a targeting sequence of 24-26 bases in length. In an embodiment of Formula (I) and/or (IA), each R² is independently selected from a naturally or non-naturally occurring nucleobase, which, when taken together, forms a targeting sequence of 24-25 bases in length.

In another embodiment of Formula (I) and/or (IA), each R² is independently selected from a naturally or non-naturally occurring nucleobase, which, when taken together, forms a targeting sequence of 25-30 bases in length. In an embodiment of Formula (I) and/or (IA), each R² is independently selected from a naturally or non-naturally occurring nucleobase, which, when taken together, forms a targeting sequence of 25-29 bases in length. In an embodiment of Formula (I) and/or (IA), each R² is independently selected from a naturally or non-naturally occurring nucleobase, which, when taken together, forms a targeting sequence of 25-28 bases in length. In an embodiment of Formula (I) and/or (IA), each R² is independently selected from a naturally or non-naturally occurring nucleobase, which, when taken together, forms a targeting sequence of 25-27 bases in length. In an embodiment of Formula (I) and/or (IA), each R² is independently selected from a naturally or non-naturally occurring nucleobase, which, when taken together, forms a targeting sequence of 25-26 bases in length.

In another embodiment of Formula (I) and/or (IA), each R² is independently selected from a naturally or non-naturally occurring nucleobase, which, when taken together, forms a targeting sequence of 26-30 bases in length. In an embodiment of Formula (I) and/or (IA), each R² is independently selected from a naturally or non-naturally occurring nucleobase, which, when taken together, forms a targeting sequence of 26-29 bases in length. In an embodiment of Formula (I) and/or (IA), each R² is independently selected from a naturally or non-naturally occurring nucleobase, which, when taken together, forms a targeting sequence of 26-28 bases in length. In an embodiment of Formula (I) and/or (IA), each R² is independently selected from a naturally or non-naturally occurring nucleobase, which, when taken together, forms a targeting sequence of 26-27 bases in length.

In another embodiment of Formula (I) and/or (IA), each R² is independently selected from a naturally or non-naturally occurring nucleobase, which, when taken together, forms a targeting sequence of 27-30 bases in length. In an embodiment of Formula (I) and/or (IA), each R² is independently selected from a naturally or non-naturally occurring nucleobase, which, when taken together, forms a targeting sequence of 27-29 bases in length. In an embodiment of Formula (I) and/or (IA), each R² is independently selected from a naturally or non-naturally occurring nucleobase, which, when taken together, forms a targeting sequence of 27-28 bases in length.

In another embodiment of Formula (I) and/or (IA), each R² is independently selected from a naturally or non-naturally occurring nucleobase, which, when taken together, forms a targeting sequence of 28-30 bases in length. In an embodiment of Formula (I) and/or (IA), each R² is independently selected from a naturally or non-naturally occurring nucleobase, which, when taken together, forms a targeting sequence of 28-29 bases in length. In another embodiment of Formula (I) and/or (IA), each R² is independently selected from a naturally or non-naturally occurring nucleobase, which, when taken together, forms a targeting sequence of 28-30 bases in length. In an embodiment of Formula (I) and/or (IA), each R² is independently selected from a naturally or non-naturally occurring nucleobase, which, when taken together, forms a targeting sequence of 29-30 bases in length.

In another embodiment of Formula (I) and/or (IA), each R² is independently selected from a naturally or non-naturally occurring nucleobase, which, when taken together, forms a targeting sequence of 13 bases in length.

In another embodiment of Formula (I) and/or (IA), each R² is independently selected from a naturally or non-naturally occurring nucleobase, which, when taken together, forms a targeting sequence of 14 bases in length. In another embodiment of Formula (I) and/or (IA), each R² is independently selected from a naturally or non-naturally occurring nucleobase, which, when taken together, forms a targeting sequence of 15 bases in length. In another embodiment of Formula (I) and/or (IA), each R² is independently selected from a naturally or non-naturally occurring nucleobase, which, when taken together, forms a targeting sequence of 16 bases in length. In another embodiment of Formula (I) and/or (IA), each R² is independently selected from a naturally or non-naturally occurring nucleobase, which, when taken together, forms a targeting sequence of 17 bases in length. In another embodiment of Formula (I) and/or (IA), each R² is independently selected from a naturally or non-naturally occurring nucleobase, which, when taken together, forms a targeting sequence of 18 bases in length. In another embodiment of Formula (I) and/or (IA), each R² is independently selected from a naturally or non-naturally occurring nucleobase, which, when taken together, forms a targeting sequence of 19 bases in length. In another embodiment of Formula (I) and/or (IA), each R² is independently selected from a naturally or non-naturally occurring nucleobase, which, when taken together, forms a targeting sequence of 20 bases in length. In another embodiment of Formula (I) and/or (IA), each R² is independently selected from a naturally or non-naturally occurring nucleobase, which, when taken together, forms a targeting sequence of 21 bases in length. In another embodiment of Formula (I) and/or (IA), each R² is independently selected from a naturally or non-naturally occurring nucleobase, which, when taken together, forms a targeting sequence of 22 bases in length. In another embodiment of Formula (I) and/or (IA), each R² is independently selected from a naturally or non-naturally occurring nucleobase, which, when taken together, forms a targeting sequence of 23 bases in length. In another embodiment of Formula (I) and/or (IA), each R² is independently selected from a naturally or non-naturally occurring nucleobase, which, when taken together, forms a targeting sequence of 24 bases in length. In another embodiment of Formula (I) and/or (IA), each R² is independently selected from a naturally or non-naturally occurring nucleobase, which, when taken together, forms a targeting sequence of 25 bases in length. In another embodiment of Formula (I) and/or (IA), each R² is independently selected from a naturally or non-naturally occurring nucleobase, which, when taken together, forms a targeting sequence of 26 bases in length. In another embodiment of Formula (I) and/or (IA), each R² is independently selected from a naturally or non-naturally occurring nucleobase, which, when taken together, forms a targeting sequence of 27 bases in length. In another embodiment of Formula (I) and/or (IA), each R² is independently selected from a naturally or non-naturally occurring nucleobase, which, when taken together, forms a targeting sequence of 28 bases in length. In another embodiment of Formula (I) and/or (IA), each R² is independently selected from a naturally or non-naturally occurring nucleobase, which, when taken together, forms a targeting sequence of 29 bases in length. In another embodiment of Formula (I) and/or (IA), each R² is independently selected from a naturally or non-naturally occurring nucleobase, which, when taken together, forms a targeting sequence of 30 bases in length.

In an embodiment of Formula (I) and/or (IA), each R², taken together, forms a targeting sequence comprising SEQ ID NO: 37. In an embodiment of Formula (I) and/or (IA), each R², taken together, forms a targeting sequence comprising SEQ ID NO: 32. In an embodiment of Formula (I) and/or (IA), each R², taken together, forms a targeting sequence comprising SEQ ID NO: 70. In an embodiment of Formula (I) and/or (IA), each R², taken together, forms a targeting sequence comprising SEQ ID NO: 74. In an embodiment of Formula (I) and/or (IA), R², taken together, forms a targeting sequence comprising SEQ ID NO: 62. In an embodiment of Formula (I) and/or (IA), each R², taken together, forms a targeting sequence comprising SEQ ID NO: 81. In an embodiment of Formula (I) and/or (IA), each R², taken together, forms a targeting sequence comprising SEQ ID NO: 114. In an embodiment of Formula (I) and/or (IA), each R², taken together, forms a targeting sequence comprising SEQ ID NO: 120.

In an embodiment, J is selected from rTAT (SEQ ID NO: 179), TAT (SEQ ID NO: 180), R₉F₂ (SEQ ID NO: 181), R₅F₂R₄ (SEQ ID NO: 182), R₄ (SEQ ID NO: 183), R₅ (SEQ ID NO: 184), R₆ (SEQ ID NO: 185), R₇ (SEQ ID NO: 136), R₈ (SEQ ID NO: 137), R₉ (SEQ ID NO: 138), (RXR)₄ (SEQ ID NO: 139), (RXR)₅ (SEQ ID NO: 140), (RXRRBR)₂ (SEQ ID NO: 141), (RAR)₄F₂ (SEQ ID NO: 142), (RGR)₄F₂ (SEQ ID NO: 143), and RBRBYLIQFRBRRBR (SEQ ID NO: 144), wherein A represents alanine, B represents β-alanine (also represented as β-Ala or β), F represents phenylalanine, G represents glycine, R represents arginine, and X represents 6-aminohexanoic acid (also represented as Ahx or α).

In an embodiment, G is selected from H, C(O)CH₃, benzoyl, and stearoyl. In an embodiment, G is H or —C(O) CH₃. In an embodiment, G is H. In an embodiment, G is —C(O)CH₃.

In some embodiments, the antisense oligomer, or a pharmaceutically acceptable salt thereof, of Formulae (I) and/or (IA) comprises a targeting sequence complementary to a target region within a pre-mRNA of the human UMOD gene. In another embodiment, the antisense oligomer, or a pharmaceutically acceptable salt thereof, comprises a targeting sequence complementary to a target region within a pre-mRNA of the human UMOD gene. In certain embodiments, the target region is an intron/exon junction or an exon internal region of exon 2 (SEQ ID NO: 2), exon 5 (SEQ ID NO: 3), exon 6 (SEQ ID NO: 4), exon 8 (SEQ ID NO: 5) or exon 9 (SEQ ID NO: 6).

In some embodiments, the target region is an intron/exon junction of the human UMOD gene pre-mRNA. In other embodiments, the target region is an exon internal region of the human UMOD gene pre-mRNA.

In an embodiment, the target region is an intron/exon junction or an exon internal region of exon 2. In some embodiments, the target region is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175). In certain embodiments, the antisense oligomer, or a pharmaceutically acceptable salt thereof, of Formulae (I) and/or (IA), or a pharmaceutically acceptable salt thereof, has a targeting sequence comprising a sequence selected from:

SEQ ID NO: 11
(GTCTGGTGTCCTGTGAACAGAGATG);
SEQ ID NO: 12
(CTCTCTGTCTCTGATGTCTGGTGTC);
SEQ ID NO: 13
(AGACGGGTTGGCCCTTTGAATTTTT);
SEQ ID NO: 14
(TCTAGATAGCACCTGCCCAAAGGAA);
SEQ ID NO: 15
(ATCCTTTCTGCTCTTCCCGC);
SEQ ID NO: 16
(CATCCTTTCTGCTCTTCCCG);
SEQ ID NO: 17
(AGAGATGGCTGCCCCATCCTTTCTG);
SEQ ID NO: 20
(CAAGTCAGAGATGGCTGCCCCATCC);
SEQ ID NO: 23
(CATCCAAGTCAGAGATGGCT);
SEQ ID NO: 26
(ACCATCAGCATCCAAGTCAG);
SEQ ID NO: 27
(AGAGGCCACCACCACCATCAGCATC);
and
SEQ ID NO: 28
(CAGTGGCTGCAGTTGTGATGAACCA).

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 51^{st} nucleotide to the 119th nucleotide, as measured from the 5′ end of exon 2, of the human UMOD gene pre-mRNA. In certain embodiments, the antisense oligomers, or pharmaceutically acceptable salts thereof, of Formulae (I) and/or (IA), have a targeting sequence complementary to a target region comprised in SEQ ID NO: 154. In other embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 51^{st} nucleotide to the 105th nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 155. In still a further embodiment, the target region is H2A(+51+75). In certain embodiments, the antisense oligomer, or a pharmaceutically acceptable salt thereof, has a targeting sequence comprising or consisting of SEQ ID NO: 14.

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 85th nucleotide to the 119th nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 156. In a further embodiment, the target region is selected from H2A(+85+104), H2A(+86+105), and H2A(+95+119). In still a further embodiment, the antisense oligomer, or a pharmaceutically acceptable salt thereof, has a targeting sequence comprising or consisting of a sequence selected from SEQ ID NOs: 15-17.

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 15th nucleotide of intron 1, as measured from the 5′ end of exon 2, and the 25th nucleotide of exon 2, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 157. In a further embodiment, the target region is selected from H2A(−15+10) and H2A(+1+25). In some embodiments, the antisense oligomer, or a pharmaceutically acceptable salt thereof, has a targeting sequence comprising or consisting of SEQ ID NO: 12 or 13.

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 118th nucleotide to the 150th nucleotide of exon 2, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 158. In a further embodiment, the target region is selected from H2A(+118+137) and H2A(+126+150). In certain embodiments, the antisense oligomers, or pharmaceutically acceptable salts thereof, have a targeting sequence comprising or consisting of SEQ ID NO: 26 or 27.

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region of exon 2 selected from H2A(+101+125), H2A(+110+129), and H2A(+151+175). In a further embodiment, the antisense oligomer, or a pharmaceutically acceptable salt thereof, has a targeting sequence comprising or consisting of a sequence selected from SEQ ID NOs: 20, 21, and 28.

In another embodiment, the target region is an intron/exon junction or an exon internal region of exon 5. In some embodiments, the target region is selected from H5A(−15+5), H5A(−14+6), H5A(−11+9), H5A(−10+10), H5A(+51+75), H5A(+76+100), H5A(+78+95), H5A(+80+97), H5A(+82+99), H5A(+126+150), H5A(+153+172), H5A(+154+173), H5D(+18-2), H5D(+16-4), H5D(+14-6), H5D(+13-7), and H5D(+12-8). In certain embodiments, the antisense oligomer, or a pharmaceutically acceptable salt thereof, of Formula (IA) has a targeting sequence comprising a

SEQ ID NO: 30
(GATATCTGAAACAGGTTAGG);
SEQ ID NO: 31
(AGATATCTGAAACAGGTTAG);
SEQ ID NO: 32
(GGGAGATATCTGAAACAGGT);
SEQ ID NO: 33
(AGGGAGATATCTGAAACAGG);
SEQ ID NO: 35
(TCAGCTGGCACTTGCCCAGCGACAC);
SEQ ID NO: 37
(AAGACCTTGTCGAAGCCCAGACTCT);
SEQ ID NO: 38
(CTTGTCGAAGCCCAGACT);
SEQ ID NO: 39
(ACCTTGTCGAAGCCCAGA);
SEQ ID NO: 40
(AGACCTTGTCGAAGCCCA);
SEQ ID NO: 44
(GGTTGTCTCTGTCATTGAAGCCCGA);
SEQ ID NO: 46
(GTCACTACAGACACCCAGTC);
SEQ ID NO: 47
(GGTCACTACAGACACCCAGT);
SEQ ID NO: 48
(ACCGTCAACACTGTCCCACA);
SEQ ID NO: 50
(GTACCGTCAACACTGTCCCA);
SEQ ID NO: 51
(ACGTACCGTCAACACTGTCC);
SEQ ID NO: 52
(GACGTACCGTCAACACTGTC);
and
SEQ ID NO: 53
(GGACGTACCGTCAACACTGT).

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 15th nucleotide of intron 4, as measured from the 5′ end of exon 5 and the 10th nucleotide of exon 5, as measured from the 5′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 159. In a further embodiment, the target region is selected from H5A(−15+5), H5A(−14+6), H5A(−11+9), and H5A(−10+10). In still a further embodiment, the antisense oligomers, or pharmaceutically acceptable salts thereof, disclosed herein, including antisense oligomers, or pharmaceutically acceptable salts thereof, of Formulae (I) and/or (IA), have a targeting sequence comprising or consisting of a sequence selected from SEQ ID NOs: 30-33.

In an embodiment, the target region is H5A(−15+5). In another embodiment, the targeting sequence comprises SEQ ID NO: 30.

In an embodiment, the target region is H5A(−14+6). In another embodiment, the target region is H5A(−15+5). In certain embodiments the targeting sequence comprises SEQ ID NO: 31.

In an embodiment, the target region is H5A(−11+9). In another embodiment, the target region is H5A(−11+9). In some embodiments, the targeting sequence comprises SEQ ID NO: 32.

In an embodiment, the target region is H5A(−10+10). In another embodiment, the targeting sequence comprises SEQ ID NO: 33.

In an embodiment, antisense oligomers, or pharmaceutically acceptable salts thereof, disclosed herein, including antisense oligomers, or pharmaceutically acceptable salts thereof, of Formulae (I) and/or (IA), have a targeting sequence complementary to the target region H5A(+51+75). In a further embodiment, antisense oligomers, or pharmaceutically acceptable salts thereof, disclosed herein, including antisense oligomers, or pharmaceutically acceptable salts thereof, of Formulae (I) and/or (IA), the targeting sequence comprises SEQ ID NO: 35.

In an embodiment, antisense oligomers, or pharmaceutically acceptable salts thereof, of the disclosed herein, including antisense oligomers, or pharmaceutically acceptable salts thereof, of Formulae (I) and/or (IA), have a targeting sequence complementary to a target region within the 76th nucleotide to the 100th nucleotide, as measured from the 5′ end of exon 5, of the human UMOD gene pre-mRNA. In certain embodiments, the antisense oligomers, or pharmaceutically acceptable salts thereof, of Formulae (I) and/or (IA), have a targeting sequence complementary to a target region comprised in SEQ ID NO: 37. In a further embodiment, the target region is selected from H5A(+76+100), H5A(+78+95), H5A(+80+97), and H5A(+82+99). In one embodiment, the target region is H5A(+76+100). In another embodiment, the targeting sequence comprises SEQ ID NO: 37.

In one embodiment, the target region is H5A(+78+95). In another embodiment, the antisense oligomer, or a pharmaceutically acceptable salt thereof, has a targeting sequence comprising SEQ ID NO: 38.

In one embodiment, the target region is H5A(+80+97). In another embodiment, the antisense oligomer, or a pharmaceutically acceptable salt thereof, has a targeting sequence comprising SEQ ID NO: 39.

In one embodiment, the target region is H5A(+82+99). In another embodiment, the antisense oligomer, or a pharmaceutically acceptable salt thereof, has a targeting sequence comprising SEQ ID NO: 40.

In an embodiment, antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 18th nucleotide of exon 5 measured from 3′ end of exon 5 to the 8th nucleotide of intron 5 measured from 3′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 160. In a further embodiment, the target region is selected from H5D(+18-2), H5D(+16-4), H5D(+14-6), H5D(+13-7), and H5D(+12-8). In still a further embodiment, antisense oligomers, or pharmaceutically acceptable salts thereof, disclosed herein, including antisense oligomers, or pharmaceutically acceptable salts thereof, of Formulae (I) and/or (IA), have a targeting sequence comprising a sequence selected from SEQ ID NOs: 48 and 50-53.

In another embodiment, the target region is an intron/exon junction or an exon internal region of exon 6. In some embodiments, the target region is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D(+24-1), H6D(+15-5), and H6D(+14-6). In certain embodiments, the antisense oligomer, or a pharmaceutically acceptable salt thereof, of Formulae (I) and/or (IA), or a pharmaceutically acceptable salt thereof, has a targeting sequence comprising a

SEQ ID NO: 57
(TCATCTGCCAGGTAGAGGGTGTTGC);
SEQ ID NO: 58
(GGTCACGGATGATGATCTCA);
SEQ ID NO: 59
(AGGTCACGGATGATGATCTC);
SEQ ID NO: 61
(TTGATGTTGAGGTCACGGAT);
SEQ ID NO: 62
(TTTGATGTTGAGGTCACGGA);
SEQ ID NO: 64
(GGTAGGAGCATGCAAAGTTGATTTT);
SEQ ID NO: 66
(TTCAGGCTGACTTTCATGTCCAGGG);
SEQ ID NO: 67
(GCTGACTTTCATGTCCAGGG);
SEQ ID NO: 68
(GGTCTTCAGGCTGACTTTCA);
SEQ ID NO: 69
(CGGTCTTCAGGCTGACTTTC);
SEQ ID NO: 70
(GCGGTCTTCAGGCTGACTTT);
SEQ ID NO: 71
(GGCGGTCTTCAGGCTGACTT);
SEQ ID NO: 72
(CTGTAGGGCGGTCTTCAGGC);
SEQ ID NO: 73
(GCTGTAGGGCGGTCTTCAGG);
SEQ ID NO: 74
(GGCTGTAGGGCGGTCTTCAG);
SEQ ID NO: 75
(TGGCTGTAGGGCGGTCTTCA);
SEQ ID NO: 76
(TTGGCTGTAGGGCGGTCTTC);
SEQ ID NO: 77
(ATTGGCTGTAGGGCGGTCTT);
SEQ ID NO: 78
(CTGACCATTGGCTGTAGGGC);
SEQ ID NO: 79
(CCTGACCATTGGCTGTAGGGCGGTC);
SEQ ID NO: 80
(CACACCTGACCATTGGCTGT);
and
SEQ ID NO: 81
(CCACACCTGACCATTGGCTG).

In an embodiment, antisense oligomers, or pharmaceutically acceptable salts thereof, disclosed herein, including antisense oligomers, or pharmaceutically acceptable salts thereof, of Formulae (I) and/or (IA), have a targeting sequence complementary to the target region H6A(+26+50). In a further embodiment, antisense oligomers, or pharmaceutically acceptable salts thereof, antisense oligomers, or pharmaceutically acceptable salts thereof, disclosed herein, including antisense oligomers, or pharmaceutically acceptable salts thereof, of Formulae (I) and/or (IA), the targeting sequence comprises SEQ ID NO: 57.

In an embodiment, antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 48th nucleotide to the 78th nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 161. In a further embodiment, the target region is selected from H6A(+48+67), H6A(+49+68), H6A(+58+77), and H6A(+59+78). In still a further embodiment, the antisense oligomer, or a pharmaceutically acceptable salt thereof, has a targeting sequence comprising a sequence selected from SEQ ID NOs: 58, 59, 61, and 62.

In another embodiment, the antisense oligomers, or pharmaceutically acceptable salts thereof, of Formulae (I) and/or (IA), have a targeting sequence complementary to a target region within the 58th nucleotide to the 78th nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 162. In certain embodiments, the target region is selected from H6A(+58+77) and H6A(+59+78). In one embodiment, the target region is H6A(+58+77). In one embodiment, the antisense oligomer, or a pharmaceutically acceptable salt thereof, has a targeting sequence comprising SEQ ID NO: 61. In another embodiment, the target region is H6A(+59+78). In one embodiment, the antisense oligomer, or a pharmaceutically acceptable salt thereof, has a targeting sequence comprising SEQ ID NO: 62.

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to the target region H6A(+76+100). In a further embodiment, the antisense oligomer, or a pharmaceutically acceptable salt thereof, has a targeting sequence complementary to the target region H6A(+26+50). In another embodiment, the targeting sequence comprises SEQ ID NO: 64.

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 101^{st} nucleotide to the 132^{nd} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 163. In a further embodiment, the target region is selected from H6A(+101+125), H6A(+101+120), H6A(+110+129), H6A(+111+130), H6A(+112+131), and H6A(+113+132). In still a further embodiment, the antisense oligomers, or pharmaceutically acceptable salts thereof, disclosed herein, including antisense oligomers, or pharmaceutically acceptable salts thereof, of Formulae (I) and/or (IA), have a targeting sequence comprising a sequence selected from SEQ ID NOs: 66-71.

In another embodiment, the antisense oligomers, or pharmaceutically acceptable salts thereof, of Formulae (I) and/or (IA), have a targeting sequence complementary to a target region within the 111th nucleotide to the 132^{nd} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 164. In certain embodiments, the target region is selected from H6A(+111+130), H6A(+112+131), and H6A(+113+132). In one embodiment, the target region Is H6A(+111+130). In one embodiment, the antisense oligomer, or a pharmaceutically acceptable salt thereof, has a targeting sequence comprising SEQ ID NO: 69. In another embodiment, the target region is H6A(+112+131). In certain embodiments and the antisense oligomer, or a pharmaceutically acceptable salt thereof, has a targeting sequence comprising SEQ ID NO: 70. In yet another embodiment, the target region is H6A(+113+133). In certain embodiments, the antisense oligomer, or a pharmaceutically acceptable salt thereof, has a targeting sequence comprising SEQ ID NO: 71.

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 119th nucleotide to the 149th nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 165. In another embodiment, the target region is selected from H6A(+119+138), H6A(+120+139), H6A(+121+140), H6A(+122+141), H6A(+123+142), H6A(+124+143), and H6A(+130+149). In yet another embodiment, the antisense oligomer, or a pharmaceutically acceptable salt thereof, has a targeting sequence comprising a sequence selected from SEQ ID NOs: 72-78.

In a further embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 119th nucleotide to the 141^{st} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 166. In an embodiment, the target region is selected from H6A(+119+138), H6A(+120+139), H6A(+121+140), and H6A(+122+141). In another embodiment, the antisense oligomer, or a pharmaceutically acceptable salt thereof, has a targeting sequence comprising a sequence selected from SEQ ID NOs: 72-75.

In still a further embodiment, the antisense oligomers, or pharmaceutically acceptable salts thereof, of Formulae (I) and/or (IA), have a targeting sequence complementary to a target region within the 120th nucleotide to the 141^{st} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 167. In some embodiments, the target region is H6A(+120+139), H6A(+121+140), or H6A(+122+141). In one embodiment, the target region is H6A(+120+139). In another embodiment, the antisense oligomer, or a pharmaceutically acceptable salt thereof, has a targeting sequence comprising SEQ ID NO: 73. In another embodiment, the target region is H6A(+121+140). In certain embodiments, the antisense oligomer, or a pharmaceutically acceptable salt thereof, has a targeting sequence comprising SEQ ID NO: 74. In yet another embodiment, the target region is H6A(+122+141). In certain embodiments, the antisense oligomer, or a pharmaceutically acceptable salt thereof, has a targeting sequence comprising SEQ ID NO: 75.

In still a further embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 123^{rd} nucleotide to the 149th nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 168. In an embodiment, the target region is selected from H6A(+123+142), H6A(+124+143), and H6A(+130+149). In another embodiment, the antisense oligomer, or a pharmaceutically acceptable salt thereof, has a targeting sequence comprising a sequence selected from SEQ ID NOs: 76-78.

In an embodiment, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region within the 24th nucleotide of exon 6 measured from 3′ end of exon 6 to the 6th nucleotide of intron 6 measured from 3′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 169. In a further embodiment, the target region is selected from H6D(+24-1), H6D(+15-5), and H6D(+14-6). In still a further embodiment, the antisense oligomer, or a pharmaceutically acceptable salt thereof, has a targeting sequence comprising a sequence selected from SEQ ID NOs: 79-81.

In a further embodiment, the antisense oligomers, or pharmaceutically acceptable salts thereof, of Formulae (I) and/or (IA), have a targeting sequence complementary to a target region within the 15th nucleotide of exon 6 measured from 3′ end of exon 6 to the 6th nucleotide of intron 6 measured from 3′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formulae (I) and/or (IA), have a targeting sequence complementary to a target region comprised in SEQ ID NO: 170. In some embodiments, the target region is H6D(+15-5), or H6D(+14-6). In one embodiment, the target region is H6D(+15-5). In certain embodiments, the antisense oligomer, or a pharmaceutically acceptable salt thereof, has a targeting sequence comprising SEQ ID NO: 80. In another embodiment, the target region is H6D(+14-6). In certain embodiments, the antisense oligomer, or a pharmaceutically acceptable salt thereof, has a targeting sequence comprising SEQ ID NO: 81.

In yet another embodiment, the target region is an intron/exon junction or an exon internal region of exon 8. In some embodiments, the target region is selected from H8A(−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D(+12-13). In certain embodiments, the antisense oligomer of Formulae (I) and/or (IA), or a pharmaceutically acceptable salt thereof, has a targeting sequence comprising a

SEQ ID NO: 82
(TTGAGTCTCTAGTGTGTGGGCATCT);
SEQ ID NO: 84
(TGGACGGAAAATCGGCCCTGGGAGG);
SEQ ID NO: 85
(CATCTGGACGGAAAATCGGC);
SEQ ID NO: 86
(ACATCTGGACGGAAAATCGG);
SEQ ID NO: 89
(AACCGGAACATCTGGACGGA);
SEQ ID NO: 90
(AAACCGGAACATCTGGACGG);
SEQ ID NO: 91
(CAAACCGGAACATCTGGACG);
SEQ ID NO: 92
(TTCCAGCAAACCGGAACATC);
SEQ ID NO: 93
(ATAGTTTCCAGCAAACCGGAACATC);
SEQ ID NO: 94
(TTTCCAGCAAACCGGAACAT);
SEQ ID NO: 95
(GTTTCCAGCAAACCGGAACA);
SEQ ID NO: 96
(AGTTTCCAGCAAACCGGAAC);
SEQ ID NO: 98
(ATAGTTTCCAGCAAACCGGA);
SEQ ID NO: 99
(CATAGTTTCCAGCAAACCGG);
SEQ ID NO: 100
(TCATAGTTTCCAGCAAACCG);
SEQ ID NO: 101
(AGGTCATAGTTTCCAGCAAA);
SEQ ID NO: 102
(GGTAGACTAGGTCATAGTTT);
SEQ ID NO: 103
(AGGTAGACTAGGTCATAGTT);
SEQ ID NO: 104
(CAGGTAGACTAGGTCATAGT);
SEQ ID NO: 105
(CTTCACAGTGCAGGTAGACTAGGTC);
SEQ ID NO: 106
(ACAGTGCAGGTAGACTAGGT);
SEQ ID NO: 107
(CACAGTGCAGGTAGACTAGG);
SEQ ID NO: 108
(TCACAGTGCAGGTAGACTAG);
SEQ ID NO: 109
(TTCACAGTGCAGGTAGACTA);
SEQ ID NO: 110
(GTCACAGAGATAGACTTCAC);
SEQ ID NO: 111
(TGTCACAGAGATAGACTTCA);
SEQ ID NO: 112
(GTGTCACAGAGATAGACTTC);
SEQ ID NO: 113
(TCATTCATGGTGTCACAGAGATAGA);
SEQ ID NO: 114
(TTCATGGTGTCACAGAGATA);
SEQ ID NO: 115
(ATTCATGGTGTCACAGAGAT);
and
SEQ ID NO: 120
(GAGTCAACTCACAGGCTTGCACTTT).

In an embodiment, the antisense oligomer, or a pharmaceutically acceptable salt thereof, has a targeting sequence complementary to the target region H8A(−2+23). In a further embodiment, the antisense oligomer, or a pharmaceutically acceptable salt thereof, has a targeting sequence comprising SEQ ID NO: 82. In still a further embodiment, the antisense oligomer, or a pharmaceutically acceptable salt thereof, has a targeting sequence complementary to the target region H8A(−2+23), wherein the targeting sequence comprises SEQ ID NO: 82.

In some embodiments, the antisense oligomer, or a pharmaceutically acceptable salt thereof, has a targeting sequence complementary to a target region within the 51^{st} nucleotide to the 80th nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 171. In a further embodiment, the target region is selected from H8A(+51+75), H8A(+60+79), and H8A(+61+80). In one embodiment, the antisense oligomer, or a pharmaceutically acceptable salt thereof, has a targeting sequence comprising a sequence selected from SEQ ID NOs: 84-86.

In an embodiment, the antisense oligomer, or a pharmaceutically acceptable salt thereof, has a targeting sequence complementary to a target region within the 68th nucleotide to the 100th nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 172. In another embodiment, the target region is selected from H8A(+68+87), H8A(+69+88), H8A(+70+89), H8A(+76+95), H8A(+76+100), H8A(+77+96), H8A(+78+97), and H8A(+79+98). In yet another embodiment, the antisense oligomer, or a pharmaceutically acceptable salt thereof, has a targeting sequence comprising a sequence selected from SEQ ID NOs: 89-96.

In a further embodiment, the antisense oligomer, or a pharmaceutically acceptable salt thereof, has a targeting sequence complementary to a target region within the 76th nucleotide to the 100th nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 173 . . . . In an embodiment, the target region is selected from H8A(+76+95), H8A(+77+96), H8A(+78+97), and H8A(+79+98). In another embodiment, the antisense oligomer, or a pharmaceutically acceptable salt thereof, has a targeting sequence comprising a sequence selected from SEQ ID NOs: 92 and 94-96.

In an embodiment, the antisense oligomer, or a pharmaceutically acceptable salt thereof, has a targeting sequence complementary to a target region within the 81^{st} nucleotide to the 105th nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 174. In a further embodiment, the target region is selected from H8A(+81+100), H8A(+82+101), H8A(+83+102), and H8A(+86+105). In still a further embodiment, the antisense oligomer, or a pharmaceutically acceptable salt thereof, has a targeting sequence comprising a sequence selected from SEQ ID NOs: 98-101.

In an embodiment, the antisense oligomer, or a pharmaceutically acceptable salt thereof, has a targeting sequence complementary to a target region within the 94th nucleotide to the 124th nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 175. In a further embodiment, the target region is selected from H8A(+94+113), H8A(+95+114), H8A(+96+115), H8A(+101+125), H8A(+102+121), H8A(+103+122), H8A(+104+123), and H8A(+105+124). In still a further embodiment, the antisense oligomer, or a pharmaceutically acceptable salt thereof, has a targeting sequence comprising a sequence selected from SEQ ID NOs: 102-109.

In an embodiment, the antisense oligomer, or a pharmaceutically acceptable salt thereof, has a targeting sequence complementary to a target region within the 120th nucleotide to the 148th nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 176. In a further embodiment, the target region is selected from H8A(+120+139), H8A(+121+140), H8A(+122+141), H8A(+126+150), H8A(+128+147), and H8A(+129+148). In still a further embodiment, the antisense oligomer, or a pharmaceutically acceptable salt thereof, has a targeting sequence comprising a sequence selected from SEQ ID NOs: 110-115.

In a further embodiment, the antisense oligomer, or a pharmaceutically acceptable salt thereof, has a targeting sequence complementary to a target region within the 126th nucleotide to the 148th nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 177. In certain embodiments, the target region is selected from H8A(+126+150), H8A(+128+147), and H8A(+129+148). In a further embodiment, the target region is selected from H8A(+126+150), H8A(+128+147), and H8A(+129+148). In some embodiments, the antisense oligomer, or a pharmaceutically acceptable salt thereof, has a targeting sequence comprising a sequence selected from SEQ ID NOs: 113-115. In one embodiment, the target region is H8A(+128+147). In some embodiments, the antisense oligomer, or a pharmaceutically acceptable salt thereof, has a targeting sequence comprising SEQ ID NO: 114.

In an embodiment, the antisense oligomer, or a pharmaceutically acceptable salt thereof, has a targeting sequence complementary to the target region H8D(+12-13). In another embodiment, the target region is H8D(+12-13). In some embodiments, the antisense oligomer, or a pharmaceutically acceptable salt thereof, has a targeting sequence comprising SEQ ID NO: 120.

In still another embodiment, the target region is an intron/exon junction or an exon internal region of exon 9. In some embodiments, the target region is selected from H9A(−5+20), H9A(+1+25), H9A(+51+75), and H9D(+7-18). In certain embodiments, the antisense oligomer of Formulae (I) and/or (IA), or a pharmaceutically acceptable salt thereof, has a targeting sequence comprising a sequence selected from:

SEQ ID NO: 121
(AATCTGGTCCCAGAGCAGGTCTACA);
SEQ ID NO: 122
(TTCGGAATCTGGTCCCAGAGCAGGT);
SEQ ID NO: 123
(TGTGATGGGACCCAAGTTCAGGACA);
and
SEQ ID NO: 124
(AGCGAGTGGCTCTCTTACCTTTCCG).

In an embodiment, the antisense oligomer, or a pharmaceutically acceptable salt thereof, has a targeting sequence complementary to a target region within the 5th nucleotide of intron 8 measured from the 5′ end of exon 9 and the 25th nucleotide of exon 9 measured from the 5′ end of exon 9 of the human UMOD gene pre-mRNA. In certain embodiments, the antisense oligomer, or a pharmaceutically acceptable salt thereof, has a targeting sequence complementary to a target region comprised in SEQ ID NO: 178. In a further embodiment, the target region is H9A(−5+20) or H9A(+1+25). In still a further embodiment, the antisense oligomer, or a pharmaceutically acceptable salt thereof, has a targeting sequence comprising a sequence selected from SEQ ID NOs: 121 and 122.

In an embodiment, the antisense oligomer, or a pharmaceutically acceptable salt thereof, has a target sequence complementary to the target region H9A(+51+75). In some embodiments, the target region is H9A(+51+75). In some embodiments, the antisense oligomer, or a pharmaceutically acceptable salt thereof, has a targeting sequence comprising SEQ ID NO: 123.

In another embodiment, the antisense oligomer, or a pharmaceutically acceptable salt thereof, has a target sequence complementary to the target region H9D(+7-18). In some embodiments, the target region is H9D(+7-18). In some embodiments, the antisense oligomer, or a pharmaceutically acceptable salt thereof, has a targeting sequence comprising SEQ ID NO: 124.

In an embodiment, the antisense oligomer of Formulae (I) and/or (IA), or a pharmaceutically acceptable salt thereof, has a targeting sequence complementary to a region within an intron/exon junction or an exon internal junction of the human UMOD gene pre-mRNA, wherein the targeting sequence comprises or consists of a sequence selected from:

| SEQ ID NO: 32 | |
|---|---|
| | (GGGAGATATCTGAAACAGGT); |
| SEQ ID NO: 37 | |
| | (AAGACCTTGTCGAAGCCCAGACTCT); |
| SEQ ID NO: 62 | |
| | (TTTGATGTTGAGGTCACGGA); |
| SEQ ID NO: 70 | |
| | (GCGGTCTTCAGGCTGACTTT); |
| SEQ ID NO: 74 | |
| | (GGCTGTAGGGCGGTCTTCAG); |
| SEQ ID NO: 81 | |
| | (CCACACCTGACCATTGGCTG); |
| SEQ ID NO: 114 | |
| | (TTCATGGTGTCACAGAGATA); |
| | and |
| SEQ ID NO: 120 | |
| | (GAGTCAACTCACAGGCTTGCACTTT). |

### Formula (II)

In some embodiments, the antisense oligomer, or a pharmaceutically acceptable salt thereof, of Formula (I) is an antisense oligomer, or a pharmaceutically acceptable salt thereof, of Formula (II):

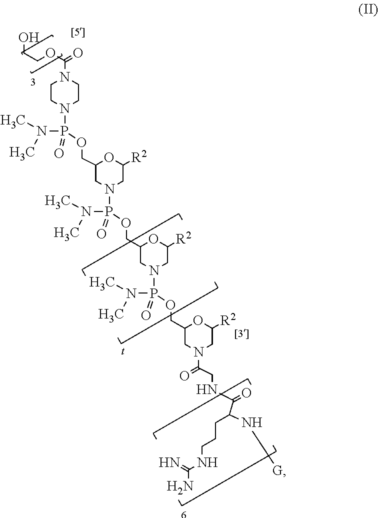

or a pharmaceutically acceptable salt thereof,
wherein:
each R² is independently selected from a naturally or non-naturally occurring nucleobase, which, when taken together, forms a targeting sequence of 13 to 30 bases in length that is complementary to a target region within a pre-mRNA of the human uromodulin (UMOD) gene represented by SEQ ID NO: 1, wherein the target region is an intron/exon junction or an exon internal region of the human UMOD gene pre-mRNA;
t is 11-28; and
G is selected from —H, —C(O)C₁₋₆-alkyl, benzoyl, and stearoyl.

In an embodiment of Formula (II), t is an integer from 11 to 28. In an embodiment of Formula (II), t is at least 11. In an embodiment of Formula (II), t is up to 28.

In an embodiment of Formula (II), t is 11-28. In an embodiment of Formula (II), t is 11-27. In an embodiment of Formula (II), t is 11-26. In an embodiment of Formula (II), t is 11-25. In an embodiment of Formula (II), t is 11-24. In an embodiment of Formula (II), t is 11-23. In an embodiment of Formula (II), t is 11-22. In an embodiment of Formula (II), t is 11-21. In an embodiment of Formula (II), t is 11-20. In an embodiment of Formula (II), t is 11-19. In an embodiment of Formula (II), t is 11-18. In an embodiment of Formula (II), t is 11-17. In an embodiment of Formula (II), t is 11-16. In an embodiment of Formula (II), t is 11-15. In an embodiment of Formula (II), t is 11-14. In an embodiment of Formula (II), t is 11-13. In an embodiment of Formula (II), t is 11-12. In an embodiment of Formula (II), t is 12-28. In an embodiment of Formula (II), t is 12-27. In an embodiment of Formula (II), t is 12-26. In an embodiment of Formula (II), t is 12-25. In an embodiment of Formula (II), t is 12-24. In an embodiment of Formula (II), t is 12-23. In an embodiment of Formula (II), t is 12-22. In an embodiment of Formula (II), t is 12-21. In an embodiment of Formula (II), t is 12-20. In an embodiment of Formula (II), t is 12-19. In an embodiment of Formula (II), t is 12-18. In an embodiment of Formula (II), t is 12-17. In an embodiment of Formula (II), t is 12-16. In an embodiment of Formula (II), t is 12-15. In an embodiment of Formula (II), t is 12-14. In an embodiment of Formula (II), t is 12-13. In an embodiment of Formula (II), t is 13-28. In an embodiment of Formula (II), t is 13-27. In an embodiment of Formula (II), t is 13-26. In an embodiment of Formula (II), t is 13-25. In an embodiment of Formula (II), t is 13-24. In an embodiment of Formula (II), t is 13-23. In an embodiment of Formula (II), t is 13-22. In an embodiment of Formula (II), t is 13-21. In an embodiment of Formula (II), t is 13-20. In an embodiment of Formula (II), t is 13-19. In an embodiment of Formula (II), t is 13-18. In an embodiment of Formula (II), t is 13-17. In an embodiment of Formula (II), t is 13-16. In an embodiment of Formula (II), t is 13-15. In an embodiment of Formula (II), t is 13-14. In an embodiment of Formula (II), t is 14-28. In an embodiment of Formula (II), t is 14-27. In an embodiment of Formula (II), t is 14-26. In an embodiment of Formula (II), t is 14-25. In an embodiment of Formula (II), t is 14-24. In an embodiment of Formula (II), t is 14-23. In an embodiment of Formula (II), t is 14-22. In an embodiment of Formula (II), t is 14-21. In an embodiment of Formula (II), t is 14-20. In an embodiment of Formula (II), t is 14-19. In an embodiment of Formula (II), t is 14-18. In an embodiment of Formula (II), t is 14-17. In an embodiment of Formula (II), t is 14-16. In an embodiment of Formula (II), t is 14-15. In an embodiment of Formula (II), t is 15-28. In an embodiment of Formula (II), t is 15-27. In an embodiment of Formula (II), t is 15-26. In an embodiment of Formula (II), t is 15-25. In an embodiment of Formula (II), t is 15-24. In an embodiment of Formula (II), t is 15-23. In an embodiment of Formula (II), t is 15-22. In an embodiment of Formula (II), t is 15-21. In an embodiment of Formula (II), t is 15-20. In an embodiment of Formula (II), t is 15-19. In an embodiment of Formula (II), t is 15-18. In an embodiment of Formula (II), t is 15-17. In an embodiment of Formula (II), t is 15-16. In an embodiment of Formula (II), t is 16-28. In an embodiment of Formula (II), t is 16-27. In an embodiment of Formula (II), t is 16-26. In an embodiment of Formula (II), t is 16-25. In an embodiment of Formula (II), t is 16-24. In an embodiment of Formula (II), t is 16-23. In an embodiment of Formula (II), t is 16-22. In an embodiment of Formula (II), t is 16-21. In an embodiment of Formula (II), t is 16-20. In an embodiment of Formula (II), t is 16-19. In an embodiment of Formula (II), t is 16-18. In an embodiment of Formula (II), t is 16-17. In an embodiment of Formula (II), t is 17-28. In an embodiment of Formula (II), t is 17-27. In an embodiment of Formula (II), t is 17-26. In an embodiment of Formula (II), t is 17-25. In an embodiment of Formula (II), t is 17-24. In an embodiment of Formula (II), t is 17-23. In an embodiment of Formula (II), t is 17-22. In an embodiment of Formula (II), t is 17-21. In an embodiment of Formula (II), t is 17-20. In an embodiment of Formula (II), t is 17-19. In an embodiment of Formula (II), t is 17-18. In an embodiment of Formula (II), t is 18-28. In an embodiment of Formula (II), t is 18-27. In an embodiment of Formula (II), t is 18-26. In an embodiment of Formula (II), t is 18-25. In an embodiment of Formula (II), t is 18-24. In an embodiment of Formula (II), t is 18-23. In an embodiment of Formula (II), t is 18-22. In an embodiment of Formula (II), t is 18-21. In an embodiment of Formula (II), t is 18-20. In an embodiment of Formula (II), t is 18-19. In an embodiment of Formula (II), t is 19-28. In an embodiment of Formula (II), t is 19-27. In an embodiment of Formula (II), t is 19-26. In an embodiment of Formula (II), t is 19-25. In an embodiment of Formula (II), t is 19-24. In an embodiment of Formula (II), t is 19-23. In an embodiment of Formula (II), t is 19-22. In an embodiment of Formula (II), t is 19-21. In an embodiment of Formula (II), t is 19-20. In an embodiment of Formula (II), t is 20-28. In an embodiment of Formula (II), t is 20-27. In an embodiment of Formula (II), t is 20-26. In an embodiment of Formula (II), t is 20-25. In an embodiment of Formula (II), t is 20-24. In an embodiment of Formula (II), t is 20-23. In an embodiment of Formula (II), t is 20-22. In an embodiment of Formula (II), t is 20-21. In an embodiment of Formula (II), t is 21-28. In an embodiment of Formula (II), t is 21-27. In an embodiment of Formula (II), t is 21-26. In an embodiment of Formula (II), t is 21-25. In an embodiment of Formula (II), t is 21-24. In an embodiment of Formula (II), t is 21-23. In an embodiment of Formula (II), t is 21-22. In an embodiment of Formula (II), t is 22-28. In an embodiment of Formula (II), t is 22-27. In an embodiment of Formula (II), t is 22-26. In an embodiment of Formula (II), t is 22-25. In an embodiment of Formula (II), t is 22-24. In an embodiment of Formula (II), t is 22-23. In an embodiment of Formula (II), t is 23-28. In an embodiment of Formula (II), t is 23-27. In an embodiment of Formula (II), t is 23-26. In an embodiment of Formula (II), t is 23-25. In an embodiment of Formula (II), t is 23-24. In an embodiment of Formula (II), t is 24-28. In an embodiment of Formula (II), t is 24-27. In an embodiment of Formula (II), t is 24-26. In an embodiment of Formula (II), t is 24-25. In an embodiment of Formula (II), t is 25-28. In an embodiment of Formula (II), t is 25-27. In an embodiment of Formula (II), t is 25-26. In another embodiment of Formula (II), t is 26-30. In an embodiment of Formula (II), t is 26-29. In an embodiment of Formula (II), t is 26-28. In an embodiment of Formula (II), t is 26-27. In an embodiment of Formula (II), t is 27-28.

In an embodiment of Formula (II), t is 11. In an embodiment of Formula (II), t is 12. In an embodiment of Formula (II), t is 13. In an embodiment of Formula (II), t is 14. In an embodiment of Formula (II), t is 15. In an embodiment of Formula (II), t is 16. In an embodiment of Formula (II), t is 17. In an embodiment of Formula (II), t is 18. In an embodiment of Formula (II), t is 19. In an embodiment of Formula (II), t is 20. In an embodiment of Formula (II), t is 21. In an embodiment of Formula (II), t is 22. In an embodiment of Formula (II), t is 23. In an embodiment of Formula (II), t is 24. In an embodiment of Formula (II), t is 25. In an embodiment of Formula (II), t is 26. In an embodiment of Formula (II), t is 27. In an embodiment of Formula (II), t is 28.

In an embodiment of Formula (II), each R² is independently selected from a naturally or non-naturally occurring nucleobase, which, when taken together, forms a targeting sequence of from 13 to 30 bases in length. In an embodiment of Formula (II), each R² is independently selected from a naturally or non-naturally occurring nucleobase, which, when taken together, forms a targeting sequence at least 13 bases in length. In an embodiment of Formula (II), each R² is independently selected from a naturally or non-naturally occurring nucleobase, which, when taken together, forms a targeting sequence of up to 30 bases in length.

In an embodiment of Formula (II), each R² is independently selected from a naturally or non-naturally occurring nucleobase, which, when taken together, forms a targeting sequence of 13-29 bases in length. In an embodiment of Formula (II), each R² is independently selected from a naturally or non-naturally occurring nucleobase, which, when taken together, forms a targeting sequence of 13-28 bases in length. In an embodiment of Formula (II), each R² is independently selected from a naturally or non-naturally occurring nucleobase, which, when taken together, forms a targeting sequence of 13-27 bases in length. In an embodiment of Formula (II), each R² is independently selected from a naturally or non-naturally occurring nucleobase, which, when taken together, forms a targeting sequence of 13-26 bases in length. In an embodiment of Formula (II), each R² is independently selected from a naturally or non-naturally occurring nucleobase, which, when taken together, forms a targeting sequence of 13-25 bases in length. In an embodiment of Formula (II), each R² is independently selected from a naturally or non-naturally occurring nucleobase, which, when taken together, forms a targeting sequence of 13-24 bases in length. In an embodiment of Formula (II), each R² is independently selected from a naturally or non-naturally occurring nucleobase, which, when taken together, forms a targeting sequence of 13-23 bases in length. In an embodiment of Formula (II), each R² is independently selected from a naturally or non-naturally occurring nucleobase, which, when taken together, forms a targeting sequence of 13-22 bases in length. In an embodiment of Formula (II), each R² is independently selected from a naturally or non-naturally occurring nucleobase, which, when taken together, forms a targeting sequence of 13-21 bases in length. In an embodiment of Formula (II), each R² is independently selected from a naturally or non-naturally occurring nucleobase, which, when taken together, forms a targeting sequence of 13-20 bases in length. In an embodiment of Formula (II), each R² is independently selected from a naturally or non-naturally occurring nucleobase, which, when taken together, forms a targeting sequence of 13-19 bases in length. In an embodiment of Formula (II), each R² is independently selected from a naturally or non-naturally occurring nucleobase, which, when taken together, forms a targeting sequence of 13-18 bases in length. In an embodiment of Formula (II), each R² is independently selected from a naturally or non-naturally occurring nucleobase, which, when taken together, forms a targeting sequence of 13-17 bases in length. In an embodiment of Formula (II), each R² is independently selected from a naturally or non-naturally occurring nucleobase, which, when taken together, forms a targeting sequence of 13-16 bases in length. In an embodiment of Formula (II), each R² is independently selected from a naturally or non-naturally occurring nucleobase, which, when taken together, forms a targeting sequence of 13-15 bases in length. In an embodiment of Formula (II), each R² is independently selected from a naturally or non-naturally occurring nucleobase, which, when taken together, forms a targeting sequence of 13-14 bases in length.

In another embodiment of Formula (II), each R² is independently selected from a naturally or non-naturally occurring nucleobase, which, when taken together, forms a targeting sequence of 14-30 bases in length. In an embodiment of Formula (II), each R² is independently selected from a naturally or non-naturally occurring nucleobase, which, when taken together, forms a targeting sequence of 14-29 bases in length. In an embodiment of Formula (II), each R² is independently selected from a naturally or non-naturally occurring nucleobase, which, when taken together, forms a targeting sequence of 14-28 bases in length. In an embodiment of Formula (II), each R² is independently selected from a naturally or non-naturally occurring nucleobase, which, when taken together, forms a targeting sequence of 14-27 bases in length. In an embodiment of Formula (II), each R² is independently selected from a naturally or non-naturally occurring nucleobase, which, when taken together, forms a targeting sequence of 14-26 bases in length. In an embodiment of Formula (II), each R² is independently selected from a naturally or non-naturally occurring nucleobase, which, when taken together, forms a targeting sequence of 14-25 bases in length. In an embodiment of Formula (II), each R² is independently selected from a naturally or non-naturally occurring nucleobase, which, when taken together, forms a targeting sequence of 14-24 bases in length. In an embodiment of Formula (II), each R² is independently selected from a naturally or non-naturally occurring nucleobase, which, when taken together, forms a targeting sequence of 14-23 bases in length. In an embodiment of Formula (II), each R² is independently selected from a naturally or non-naturally occurring nucleobase, which, when taken together, forms a targeting sequence of 14-22 bases in length. In an embodiment of Formula (II), each R² is independently selected from a naturally or non-naturally occurring nucleobase, which, when taken together, forms a targeting sequence of 14-21 bases in length. In an embodiment of Formula (II), each R² is independently selected from a naturally or non-naturally occurring nucleobase, which, when taken together, forms a targeting sequence of 14-20 bases in length. In an embodiment of Formula (II), each R² is independently selected from a naturally or non-naturally occurring nucleobase, which, when taken together, forms a targeting sequence of 14-19 bases in length. In an embodiment of Formula (II), each R² is independently selected from a naturally or non-naturally occurring nucleobase, which, when taken together, forms a targeting sequence of 14-18 bases in length. In an embodiment of Formula (II), each R² is independently selected from a naturally or non-naturally occurring nucleobase, which, when taken together, forms a targeting sequence of 14-17 bases in length. In an embodiment of Formula (II), each R² is independently selected from a naturally or non-naturally occurring nucleobase, which, when taken together, forms a targeting sequence of 14-16 bases in length. In an embodiment of Formula (II), each R² is independently selected from a naturally or non-naturally occurring nucleobase, which, when taken together, forms a targeting sequence of 14-15 bases in length.

In another embodiment of Formula (II), each R² is independently selected from a naturally or non-naturally occurring nucleobase, which, when taken together, forms a targeting sequence of 15-30 bases in length. In an embodiment of Formula (II), each R² is independently selected from a naturally or non-naturally occurring nucleobase, which, when taken together, forms a targeting sequence of 15-29 bases in length. In an embodiment of Formula (II), each R² is independently selected from a naturally or non-naturally occurring nucleobase, which, when taken together, forms a targeting sequence of 15-28 bases in length. In an embodiment of Formula (II), each R² is independently selected from a naturally or non-naturally occurring nucleobase, which, when taken together, forms a targeting sequence of 15-27 bases in length. In an embodiment of Formula (II), each R² is independently selected from a naturally or non-naturally occurring nucleobase, which, when taken together, forms a targeting sequence of 15-26 bases in length. In an embodiment of Formula (II), each R² is independently selected from a naturally or non-naturally occurring nucleobase, which, when taken together, forms a targeting sequence of 15-25 bases in length. In an embodiment of Formula (II), each R² is independently selected from a naturally or non-naturally occurring nucleobase, which, when taken together, forms a targeting sequence of 15-24 bases in length. In an embodiment of Formula (II), each R² is independently selected from a naturally or non-naturally occurring nucleobase, which, when taken together, forms a targeting sequence of 15-23 bases in length. In an embodiment of Formula (II), each R² is independently selected from a naturally or non-naturally occurring nucleobase, which, when taken together, forms a targeting sequence of 15-22 bases in length. In an embodiment of Formula (II), each R² is independently selected from a naturally or non-naturally occurring nucleobase, which, when taken together, forms a targeting sequence of 15-21 bases in length. In an embodiment of Formula (II), each R² is independently selected from a naturally or non-naturally occurring nucleobase, which, when taken together, forms a targeting sequence of 15-20 bases in length. In an embodiment of Formula (II), each R² is independently selected from a naturally or non-naturally occurring nucleobase, which, when taken together, forms a targeting sequence of 15-19 bases in length. In an embodiment of Formula (II), each R² is independently selected from a naturally or non-naturally occurring nucleobase, which, when taken together, forms a targeting sequence of 15-18 bases in length. In an embodiment of Formula (II), each R² is independently selected from a naturally or non-naturally occurring nucleobase, which, when taken together, forms a targeting sequence of 15-17 bases in length. In an embodiment of Formula (II), each R² is independently selected from a naturally or non-naturally occurring nucleobase, which, when taken together, forms a targeting sequence of 15-16 bases in length.

In another embodiment of Formula (II), each R² is independently selected from a naturally or non-naturally occurring nucleobase, which, when taken together, forms a targeting sequence of 16-30 bases in length. In an embodiment of Formula (II), each R² is independently selected from a naturally or non-naturally occurring nucleobase, which, when taken together, forms a targeting sequence of 16-29 bases in length. In an embodiment of Formula (II), each R² is independently selected from a naturally or non-naturally occurring nucleobase, which, when taken together, forms a targeting sequence of 16-28 bases in length. In an embodiment of Formula (II), each R² is independently selected from a naturally or non-naturally occurring nucleobase, which, when taken together, forms a targeting sequence of 16-27 bases in length. In an embodiment of Formula (II), each R² is independently selected from a naturally or non-naturally occurring nucleobase, which, when taken together, forms a targeting sequence of 16-26 bases in length. In an embodiment of Formula (II), each R² is independently selected from a naturally or non-naturally occurring nucleobase, which, when taken together, forms a targeting sequence of 16-25 bases in length. In an embodiment of Formula (II), each R² is independently selected from a naturally or non-naturally occurring nucleobase, which, when taken together, forms a targeting sequence of 16-24 bases in length. In an embodiment of Formula (II), each R² is independently selected from a naturally or non-naturally occurring nucleobase, which, when taken together, forms a targeting sequence of 16-23 bases in length. In an embodiment of Formula (II), each R² is independently selected from a naturally or non-naturally occurring nucleobase, which, when taken together, forms a targeting sequence of 16-22 bases in length. In an embodiment of Formula (II), each R² is independently selected from a naturally or non-naturally occurring nucleobase, which, when taken together, forms a targeting sequence of 16-21 bases in length. In an embodiment of Formula (II), each R² is independently selected from a naturally or non-naturally occurring nucleobase, which, when taken together, forms a targeting sequence of 16-20 bases in length. In an embodiment of Formula (II), each R² is independently selected from a naturally or non-naturally occurring nucleobase, which, when taken together, forms a targeting sequence of 16-19 bases in length. In an embodiment of Formula (II), each R² is independently selected from a naturally or non-naturally occurring nucleobase, which, when taken together, forms a targeting sequence of 16-18 bases in length. In an embodiment of Formula (II), each R² is independently selected from a naturally or non-naturally occurring nucleobase, which, when taken together, forms a targeting sequence of 16-17 bases in length.

In another embodiment of Formula (II), each R² is independently selected from a naturally or non-naturally occurring nucleobase, which, when taken together, forms a targeting sequence of 17-30 bases in length. In an embodiment of Formula (II), each R² is independently selected from a naturally or non-naturally occurring nucleobase, which, when taken together, forms a targeting sequence of 17-29 bases in length. In an embodiment of Formula (II), each R² is independently selected from a naturally or non-naturally occurring nucleobase, which, when taken together, forms a targeting sequence of 17-28 bases in length. In an embodiment of Formula (II), each R² is independently selected from a naturally or non-naturally occurring nucleobase, which, when taken together, forms a targeting sequence of 17-27 bases in length. In an embodiment of Formula (II), each R² is independently selected from a naturally or non-naturally occurring nucleobase, which, when taken together, forms a targeting sequence of 17-26 bases in length. In an embodiment of Formula (II), each R² is independently selected from a naturally or non-naturally occurring nucleobase, which, when taken together, forms a targeting sequence of 17-25 bases in length. In an embodiment of Formula (II), each R² is independently selected from a naturally or non-naturally occurring nucleobase, which, when taken together, forms a targeting sequence of 17-24 bases in length. In an embodiment of Formula (II), each R² is independently selected from a naturally or non-naturally occurring nucleobase, which, when taken together, forms a targeting sequence of 17-23 bases in length. In an embodiment of Formula (II), each R² is independently selected from a naturally or non-naturally occurring nucleobase, which, when taken together, forms a targeting sequence of 17-22 bases in length. In an embodiment of Formula (II), each R² is independently selected from a naturally or non-naturally occurring nucleobase, which, when taken together, forms a targeting sequence of 17-21 bases in length. In an embodiment of Formula (II), each R² is independently selected from a naturally or non-naturally occurring nucleobase, which, when taken together, forms a targeting sequence of 17-20 bases in length. In an embodiment of Formula (II), each R² is independently selected from a naturally or non-naturally occurring nucleobase, which, when taken together, forms a targeting sequence of 17-19 bases in length. In an embodiment of Formula (II), each R² is independently selected from a naturally or non-naturally occurring nucleobase, which, when taken together, forms a targeting sequence of 17-18 bases in length.

In another embodiment of Formula (II), each R² is independently selected from a naturally or non-naturally occurring nucleobase, which, when taken together, forms a targeting sequence of 18-30 bases in length. In an embodiment of Formula (II), each R² is independently selected from a naturally or non-naturally occurring nucleobase, which, when taken together, forms a targeting sequence of 18-29 bases in length. In an embodiment of Formula (II), each R² is independently selected from a naturally or non-naturally occurring nucleobase, which, when taken together, forms a targeting sequence of 18-28 bases in length. In an embodiment of Formula (II), each R² is independently selected from a naturally or non-naturally occurring nucleobase, which, when taken together, forms a targeting sequence of 18-27 bases in length. In an embodiment of Formula (II), each R² is independently selected from a naturally or non-naturally occurring nucleobase, which, when taken together, forms a targeting sequence of 18-26 bases in length. In an embodiment of Formula (II), each R² is independently selected from a naturally or non-naturally occurring nucleobase, which, when taken together, forms a targeting sequence of 18-25 bases in length. In an embodiment of Formula (II), each R² is independently selected from a naturally or non-naturally occurring nucleobase, which, when taken together, forms a targeting sequence of 18-24 bases in length. In an embodiment of Formula (II), each R² is independently selected from a naturally or non-naturally occurring nucleobase, which, when taken together, forms a targeting sequence of 18-23 bases in length. In an embodiment of Formula (II), each R² is independently selected from a naturally or non-naturally occurring nucleobase, which, when taken together, forms a targeting sequence of 18-22 bases in length. In an embodiment of Formula (II), each R² is independently selected from a naturally or non-naturally occurring nucleobase, which, when taken together, forms a targeting sequence of 18-21 bases in length. In an embodiment of Formula (II), each R² is independently selected from a naturally or non-naturally occurring nucleobase, which, when taken together, forms a targeting sequence of 18-20 bases in length. In an embodiment of Formula (II), each R² is independently selected from a naturally or non-naturally occurring nucleobase, which, when taken together, forms a targeting sequence of 18-19 bases in length.

In another embodiment of Formula (II), each R² is independently selected from a naturally or non-naturally occurring nucleobase, which, when taken together, forms a targeting sequence of 19-30 bases in length. In an embodiment of Formula (II), each R² is independently selected from a naturally or non-naturally occurring nucleobase, which, when taken together, forms a targeting sequence of 19-29 bases in length. In an embodiment of Formula (II), each R² is independently selected from a naturally or non-naturally occurring nucleobase, which, when taken together, forms a targeting sequence of 19-28 bases in length. In an embodiment of Formula (II), each R² is independently selected from a naturally or non-naturally occurring nucleobase, which, when taken together, forms a targeting sequence of 19-27 bases in length. In an embodiment of Formula (II), each R² is independently selected from a naturally or non-naturally occurring nucleobase, which, when taken together, forms a targeting sequence of 19-26 bases in length. In an embodiment of Formula (II), each R² is independently selected from a naturally or non-naturally occurring nucleobase, which, when taken together, forms a targeting sequence of 19-25 bases in length. In an embodiment of Formula (II), each R² is independently selected from a naturally or non-naturally occurring nucleobase, which, when taken together, forms a targeting sequence of 19-24 bases in length. In an embodiment of Formula (II), each R² is independently selected from a naturally or non-naturally occurring nucleobase, which, when taken together, forms a targeting sequence of 19-23 bases in length. In an embodiment of Formula (II), each R² is independently selected from a naturally or non-naturally occurring nucleobase, which, when taken together, forms a targeting sequence of 19-22 bases in length. In an embodiment of Formula (II), each R² is independently selected from a naturally or non-naturally occurring nucleobase, which, when taken together, forms a targeting sequence of 19-21 bases in length. In an embodiment of Formula (II), each R² is independently selected from a naturally or non-naturally occurring nucleobase, which, when taken together, forms a targeting sequence of 19-20 bases in length.

In another embodiment of Formula (II), each R² is independently selected from a naturally or non-naturally occurring nucleobase, which, when taken together, forms a targeting sequence of 20-30 bases in length. In an embodiment of Formula (II), each R² is independently selected from a naturally or non-naturally occurring nucleobase, which, when taken together, forms a targeting sequence of 20-29 bases in length. In an embodiment of Formula (II), each R² is independently selected from a naturally or non-naturally occurring nucleobase, which, when taken together, forms a targeting sequence of 20-28 bases in length. In an embodiment of Formula (II), each R² is independently selected from a naturally or non-naturally occurring nucleobase, which, when taken together, forms a targeting sequence of 20-27 bases in length. In an embodiment of Formula (II), each R² is independently selected from a naturally or non-naturally occurring nucleobase, which, when taken together, forms a targeting sequence of 20-26 bases in length. In an embodiment of Formula (II), each R² is independently selected from a naturally or non-naturally occurring nucleobase, which, when taken together, forms a targeting sequence of 20-25 bases in length. In an embodiment of Formula (II), each R² is independently selected from a naturally or non-naturally occurring nucleobase, which, when taken together, forms a targeting sequence of 20-24 bases in length. In an embodiment of Formula (II), each R² is independently selected from a naturally or non-naturally occurring nucleobase, which, when taken together, forms a targeting sequence of 20-23 bases in length. In an embodiment of Formula (II), each R² is independently selected from a naturally or non-naturally occurring nucleobase, which, when taken together, forms a targeting sequence of 20-22 bases in length. In an embodiment of Formula (II), each R² is independently selected from a naturally or non-naturally occurring nucleobase, which, when taken together, forms a targeting sequence of 20-21 bases in length.

In another embodiment of Formula (II), each R² is independently selected from a naturally or non-naturally occurring nucleobase, which, when taken together, forms a targeting sequence of 21-30 bases in length. In an embodiment of Formula (II), each R² is independently selected from a naturally or non-naturally occurring nucleobase, which, when taken together, forms a targeting sequence of 21-29 bases in length. In an embodiment of Formula (II), each R² is independently selected from a naturally or non-naturally occurring nucleobase, which, when taken together, forms a targeting sequence of 21-28 bases in length. In an embodiment of Formula (II), each R² is independently selected from a naturally or non-naturally occurring nucleobase, which, when taken together, forms a targeting sequence of 21-27 bases in length. In an embodiment of Formula (II), each R² is independently selected from a naturally or non-naturally occurring nucleobase, which, when taken together, forms a targeting sequence of 21-26 bases in length. In an embodiment of Formula (II), each R² is independently selected from a naturally or non-naturally occurring nucleobase, which, when taken together, forms a targeting sequence of 21-25 bases in length. In an embodiment of Formula (II), each R² is independently selected from a naturally or non-naturally occurring nucleobase, which, when taken together, forms a targeting sequence of 21-24 bases in length. In an embodiment of Formula (II), each R² is independently selected from a naturally or non-naturally occurring nucleobase, which, when taken together, forms a targeting sequence of 21-23 bases in length. In an embodiment of Formula (II), each R² is independently selected from a naturally or non-naturally occurring nucleobase, which, when taken together, forms a targeting sequence of 21-22 bases in length.

In another embodiment of Formula (II), each R² is independently selected from a naturally or non-naturally occurring nucleobase, which, when taken together, forms a targeting sequence of 22-30 bases in length. In an embodiment of Formula (II), each R² is independently selected from a naturally or non-naturally occurring nucleobase, which, when taken together, forms a targeting sequence of 22-29 bases in length. In an embodiment of Formula (II), each R² is independently selected from a naturally or non-naturally occurring nucleobase, which, when taken together, forms a targeting sequence of 22-28 bases in length. In an embodiment of Formula (II), each R² is independently selected from a naturally or non-naturally occurring nucleobase, which, when taken together, forms a targeting sequence of 22-27 bases in length. In an embodiment of Formula (II), each R² is independently selected from a naturally or non-naturally occurring nucleobase, which, when taken together, forms a targeting sequence of 22-26 bases in length. In an embodiment of Formula (II), each R² is independently selected from a naturally or non-naturally occurring nucleobase, which, when taken together, forms a targeting sequence of 22-25 bases in length. In an embodiment of Formula (II), each R² is independently selected from a naturally or non-naturally occurring nucleobase, which, when taken together, forms a targeting sequence of 22-24 bases in length. In an embodiment of Formula (II), each R² is independently selected from a naturally or non-naturally occurring nucleobase, which, when taken together, forms a targeting sequence of 22-23 bases in length.

In another embodiment of Formula (II), each R² is independently selected from a naturally or non-naturally occurring nucleobase, which, when taken together, forms a targeting sequence of 23-30 bases in length. In an embodiment of Formula (II), each R² is independently selected from a naturally or non-naturally occurring nucleobase, which, when taken together, forms a targeting sequence of 23-29 bases in length. In an embodiment of Formula (II), each R² is independently selected from a naturally or non-naturally occurring nucleobase, which, when taken together, forms a targeting sequence of 23-28 bases in length. In an embodiment of Formula (II), each R² is independently selected from a naturally or non-naturally occurring nucleobase, which, when taken together, forms a targeting sequence of 23-27 bases in length. In an embodiment of Formula (II), each R² is independently selected from a naturally or non-naturally occurring nucleobase, which, when taken together, forms a targeting sequence of 23-26 bases in length. In an embodiment of Formula (II), each R² is independently selected from a naturally or non-naturally occurring nucleobase, which, when taken together, forms a targeting sequence of 23-25 bases in length. In an embodiment of Formula (II), each R² is independently selected from a naturally or non-naturally occurring nucleobase, which, when taken together, forms a targeting sequence of 23-24 bases in length.

In another embodiment of Formula (II), each R² is independently selected from a naturally or non-naturally occurring nucleobase, which, when taken together, forms a targeting sequence of 24-30 bases in length. In an embodiment of Formula (II), each R² is independently selected from a naturally or non-naturally occurring nucleobase, which, when taken together, forms a targeting sequence of 24-29 bases in length. In an embodiment of Formula (II), each R² is independently selected from a naturally or non-naturally occurring nucleobase, which, when taken together, forms a targeting sequence of 24-28 bases in length. In an embodiment of Formula (II), each R² is independently selected from a naturally or non-naturally occurring nucleobase, which, when taken together, forms a targeting sequence of 24-27 bases in length. In an embodiment of Formula (II), each R² is independently selected from a naturally or non-naturally occurring nucleobase, which, when taken together, forms a targeting sequence of 24-26 bases in length. In an embodiment of Formula (II), each R² is independently selected from a naturally or non-naturally occurring nucleobase, which, when taken together, forms a targeting sequence of 24-25 bases in length.

In another embodiment of Formula (II), each R² is independently selected from a naturally or non-naturally occurring nucleobase, which, when taken together, forms a targeting sequence of 25-30 bases in length. In an embodiment of Formula (II), each R² is independently selected from a naturally or non-naturally occurring nucleobase, which, when taken together, forms a targeting sequence of 25-29 bases in length. In an embodiment of Formula (II), each R² is independently selected from a naturally or non-naturally occurring nucleobase, which, when taken together, forms a targeting sequence of 25-28 bases in length. In an embodiment of Formula (II), each R² is independently selected from a naturally or non-naturally occurring nucleobase, which, when taken together, forms a targeting sequence of 25-27 bases in length. In an embodiment of Formula (II), each R² is independently selected from a naturally or non-naturally occurring nucleobase, which, when taken together, forms a targeting sequence of 25-26 bases in length.

In another embodiment of Formula (II), each R² is independently selected from a naturally or non-naturally occurring nucleobase, which, when taken together, forms a targeting sequence of 26-30 bases in length. In an embodiment of Formula (II), each R² is independently selected from a naturally or non-naturally occurring nucleobase, which, when taken together, forms a targeting sequence of 26-29 bases in length. In an embodiment of Formula (II), each R² is independently selected from a naturally or non-naturally occurring nucleobase, which, when taken together, forms a targeting sequence of 26-28 bases in length. In an embodiment of Formula (II), each R² is independently selected from a naturally or non-naturally occurring nucleobase, which, when taken together, forms a targeting sequence of 26-27 bases in length.

In another embodiment of Formula (II), each R² is independently selected from a naturally or non-naturally occurring nucleobase, which, when taken together, forms a targeting sequence of 27-30 bases in length. In an embodiment of Formula (II), each R² is independently selected from a naturally or non-naturally occurring nucleobase, which, when taken together, forms a targeting sequence of 27-29 bases in length. In an embodiment of Formula (II), each R² is independently selected from a naturally or non-naturally occurring nucleobase, which, when taken together, forms a targeting sequence of 27-28 bases in length.

In another embodiment of Formula (II), each R² is independently selected from a naturally or non-naturally occurring nucleobase, which, when taken together, forms a targeting sequence of 28-30 bases in length. In an embodiment of Formula (II), each R² is independently selected from a naturally or non-naturally occurring nucleobase, which, when taken together, forms a targeting sequence of 28-29 bases in length. In another embodiment of Formula (II), each R² is independently selected from a naturally or non-naturally occurring nucleobase, which, when taken together, forms a targeting sequence of 28-30 bases in length. In an embodiment of Formula (II), each R² is independently selected from a naturally or non-naturally occurring nucleobase, which, when taken together, forms a targeting sequence of 29-30 bases in length.

In another embodiment of Formula (II), each R² is independently selected from a naturally or non-naturally occurring nucleobase, which, when taken together, forms a targeting sequence of 13 bases in length.

In another embodiment of Formula (II), each R² is independently selected from a naturally or non-naturally occurring nucleobase, which, when taken together, forms a targeting sequence of 14 bases in length. In another embodiment of Formula (II), each R² is independently selected from a naturally or non-naturally occurring nucleobase, which, when taken together, forms a targeting sequence of 15 bases in length. In another embodiment of Formula (II), each R² is independently selected from a naturally or non-naturally occurring nucleobase, which, when taken together, forms a targeting sequence of 16 bases in length. In another embodiment of Formula (II), each R² is independently selected from a naturally or non-naturally occurring nucleobase, which, when taken together, forms a targeting sequence of 17 bases in length. In another embodiment of Formula (II), each R² is independently selected from a naturally or non-naturally occurring nucleobase, which, when taken together, forms a targeting sequence of 18 bases in length. In another embodiment of Formula (II), each R² is independently selected from a naturally or non-naturally occurring nucleobase, which, when taken together, forms a targeting sequence of 19 bases in length. In another embodiment of Formula (II), each R² is independently selected from a naturally or non-naturally occurring nucleobase, which, when taken together, forms a targeting sequence of 20 bases in length. In another embodiment of Formula (II), each R² is independently selected from a naturally or non-naturally occurring nucleobase, which, when taken together, forms a targeting sequence of 21 bases in length. In another embodiment of Formula (II), each R² is independently selected from a naturally or non-naturally occurring nucleobase, which, when taken together, forms a targeting sequence of 22 bases in length. In another embodiment of Formula (II), each R² is independently selected from a naturally or non-naturally occurring nucleobase, which, when taken together, forms a targeting sequence of 23 bases in length. In another embodiment of Formula (II), each R² is independently selected from a naturally or non-naturally occurring nucleobase, which, when taken together, forms a targeting sequence of 24 bases in length. In another embodiment of Formula (II), each R² is independently selected from a naturally or non-naturally occurring nucleobase, which, when taken together, forms a targeting sequence of 25 bases in length. In another embodiment of Formula (II), each R² is independently selected from a naturally or non-naturally occurring nucleobase, which, when taken together, forms a targeting sequence of 26 bases in length. In another embodiment of Formula (II), each R² is independently selected from a naturally or non-naturally occurring nucleobase, which, when taken together, forms a targeting sequence of 27 bases in length. In another embodiment of Formula (II), each R² is independently selected from a naturally or non-naturally occurring nucleobase, which, when taken together, forms a targeting sequence of 28 bases in length. In another embodiment of Formula (II), each R² is independently selected from a naturally or non-naturally occurring nucleobase, which, when taken together, forms a targeting sequence of 29 bases in length. In another embodiment of Formula (II), each R² is independently selected from a naturally or non-naturally occurring nucleobase, which, when taken together, forms a targeting sequence of 30 bases in length.

In an embodiment of Formula (II), each R², taken together, forms a targeting sequence comprising SEQ ID NO: 37. In an embodiment of Formula (II), each R², taken together, forms a targeting sequence comprising SEQ ID NO: 32. In an embodiment of Formula (II), each R², taken together, forms a targeting sequence comprising SEQ ID NO: 70. In an embodiment of Formula (II), each R², taken together, forms a targeting sequence comprising SEQ ID NO: 74. In an embodiment of Formula (II), R², taken together, forms a targeting sequence comprising SEQ ID NO: 62. In an embodiment of Formula (II), each R², taken together, forms a targeting sequence comprising SEQ ID NO: 81. In an embodiment of Formula (II), each R², taken together, forms a targeting sequence comprising SEQ ID NO: 114. In an embodiment of Formula (II), each R², taken together, forms a targeting sequence comprising SEQ ID NO: 120.

In an embodiment, J is selected from rTAT (SEQ ID NO: 179), TAT (SEQ ID NO: 180), R⁹F₂ (SEQ ID NO: 181), R₅F₂R₄ (SEQ ID NO: 182), R⁴ (SEQ ID NO: 183), R⁵ (SEQ ID NO: 184), R⁶ (SEQ ID NO: 185), R⁷ (SEQ ID NO: 136), R⁸ (SEQ ID NO: 137), R⁹ (SEQ ID NO: 138), (RXR)₄ (SEQ ID NO: 139), (RXR)₅ (SEQ ID NO: 140), (RXRRBR)₂ (SEQ ID NO: 141), (RAR)₄F₂ (SEQ ID NO: 142), (RGR)₄F₂ (SEQ ID NO: 143), and RBRBYLIQFRBRRBR (SEQ ID NO: 144), wherein A represents alanine, B represents β-alanine (also represented as β-Ala or β), F represents phenylalanine, G represents glycine, R represents arginine, and X represents 6-aminohexanoic acid (also represented as Ahx or α).

In an embodiment, G is selected from H, C(O)CH₃, benzoyl, and stearoyl. In an embodiment, G is H or —C(O) CH₃. In an embodiment, G is H. In an embodiment, G is —C(O)CH₃.

In an embodiment, the antisense oligomer, or a pharmaceutically acceptable salt thereof, of Formula (II) comprises a targeting sequence complementary to a target region within a pre-mRNA of the human UMOD gene. In another embodiment, the antisense oligomer, or a pharmaceutically acceptable salt thereof, comprises a targeting sequence complementary to a target region within a pre-mRNA of the human UMOD gene. In certain embodiments, the target region is an intron/exon junction or an exon internal region of exon 2 (SEQ ID NO: 2), exon 5 (SEQ ID NO: 3), exon 6 (SEQ ID NO: 4), exon 8 (SEQ ID NO: 5), or exon 9 (SEQ ID NO: 6).

In some embodiments, the target region is an intron/exon junction of the human UMOD gene pre-mRNA. In other embodiments, the target region is an exon internal region of the human UMOD gene pre-mRNA.

In an embodiment, the target region is an intron/exon junction or an exon internal region of exon 2. In certain embodiments, the target region is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175). In certain embodiments, the antisense oligomer of Formula (II), or a pharmaceutically acceptable salt thereof, has a targeting sequence comprising a sequence selected from:

| SEQ ID NO: 11 | |
|---|---|
| | (GTCTGGTGTCCTGTGAACAGAGATG); |
| SEQ ID NO: 12 | |
| | (CTCTCTGTCTCTGATGTCTGGTGTC); |
| SEQ ID NO: 13 | |
| | (AGACGGGTTGGCCCTTTGAATTTTT); |
| SEQ ID NO: 14 | |
| | (TCTAGATAGCACCTGCCCAAAGGAA); |
| SEQ ID NO: 15 | |
| | (ATCCTTTCTGCTCTTCCCGC); |
| SEQ ID NO: 16 | |
| | (CATCCTTTCTGCTCTTCCCG); |
| SEQ ID NO: 17 | |
| | (AGAGATGGCTGCCCCATCCTTTCTG); |
| SEQ ID NO: 20 | |
| | (CAAGTCAGAGATGGCTGCCCCATCC); |
| SEQ ID NO: 23 | |
| | (CATCCAAGTCAGAGATGGCT); |
| SEQ ID NO: 26 | |
| | (ACCATCAGCATCCAAGTCAG); |
| SEQ ID NO: 27 | |
| | (AGAGGCCACCACCACCATCAGCATC); |
| | and |
| SEQ ID NO: 28 | |
| | (CAGTGGCTGCAGTTGTGATGAACCA). |

In an embodiment, the antisense oligomers, or a pharmaceutically acceptable salt thereof, disclosed herein, including antisense oligomers, or pharmaceutically acceptable salts thereof, of Formula (II), have a targeting sequence complementary to a target region within the 51^{st} nucleotide to the 119^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 154. In other embodiments, the antisense oligomers, or pharmaceutically acceptable salts thereof, disclosed herein, including antisense oligomers, or pharmaceutically acceptable salts thereof, of Formula (II), have a targeting sequence complementary to a target region within the 51^{st} nucleotide to the 105^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 155. In still a further embodiment, the target region is H2A(+51+75). In certain embodiments, the antisense oligomer, or a pharmaceutically acceptable salt thereof, has a targeting sequence comprising or consisting of SEQ ID NO: 14.

In an embodiment, the antisense oligomers, or pharmaceutically acceptable salts thereof, disclosed herein, including antisense oligomers, or pharmaceutically acceptable salts thereof, of Formula (II), have a targeting sequence complementary to a target region within the 85^{th} nucleotide to the 119^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 156. In a further embodiment, the target region is selected from H2A(+85+104), H2A(+86+105), and H2A(+95+119). In still a further embodiment, the antisense oligomer, or a pharmaceutically acceptable salt thereof, has a targeting sequence comprising or consisting of a sequence selected from SEQ ID NOs: 15-17.

In an embodiment, the antisense oligomers, or pharmaceutically acceptable salts thereof, disclosed herein, including antisense oligomers, or pharmaceutically acceptable salts thereof, of Formula (II), have a targeting sequence complementary to a target region within the 15^{th} nucleotide of intron 1, as measured from the 5′ end of exon 2, and the 25^{th} nucleotide of exon 2, as measured from 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 157. In a further embodiment, the target region is selected from H2A(−15+10) and H2A(+1+25). In some embodiments, the antisense oligomer, or a pharmaceutically acceptable salt thereof, has a targeting sequence comprising or consisting of SEQ ID NO: 12 or 13.

In an embodiment, the antisense oligomers, or pharmaceutically acceptable salts thereof, disclosed herein, including antisense oligomers, or pharmaceutically acceptable salts thereof, of Formula (II), have a targeting sequence complementary to a target region within the 118^{th} nucleotide to the 150^{th} nucleotide of exon 2, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 158. In a further embodiment, the target region is selected from H2A(+118+137) and H2A(+126+150). In certain embodiments, the antisense oligomers, or pharmaceutically acceptable salts thereof, have a targeting sequence comprising or consisting of SEQ ID NO: 26 or 27.

In an embodiment, the antisense oligomers, or pharmaceutically acceptable salts thereof, disclosed herein, including antisense oligomers, or pharmaceutically acceptable salts thereof, of Formula (II), have a targeting sequence complementary to a target region of exon 2 selected from H2A(+101+125), H2A(+110+129), and H2A(+151+175). In a further embodiment, the antisense oligomer, or a pharmaceutically acceptable salt thereof, has a targeting sequence comprising or consisting of a sequence selected from SEQ ID NOs: 20, 21, and 28.

In another embodiment, the target region is an intron/exon junction or an exon internal region of exon 5. In certain embodiments, the target region is selected from H5A(−15+5), H5A(−14+6), H5A(−11+9), H5A(−10+10), H5A(+51+75), H5A(+76+100), H5A(+78+95), H5A(+80+97), H5A(+82+99), H5A(+126+150), H5A(+153+172), H5A(+154+173), H5D(+18-2), H5D(+16-4), H5D(+14-6), H5D(+13-7), and H5D(+12-8). In certain embodiments, the antisense oligomer of Formula (II), or a pharmaceutically acceptable salt thereof, has a targeting sequence comprising a

| SEQ ID NO: 30 | |
|---|---|
| | (GATATCTGAAACAGGTTAGG); |
| SEQ ID NO: 31 | |
| | (AGATATCTGAAACAGGTTAG); |
| SEQ ID NO: 32 | |
| | (GGGAGATATCTGAAACAGGT); |
| SEQ ID NO: 33 | |
| | (AGGGAGATATCTGAAACAGG); |
| SEQ ID NO: 35 | |
| | (TCAGCTGGCACTTGCCCAGCGACAC); |
| SEQ ID NO: 37 | |
| | (AAGACCTTGTCGAAGCCCAGACTCT); |
| SEQ ID NO: 38 | |
| | (CTTGTCGAAGCCCAGACT); |
| SEQ ID NO: 39 | |
| | (ACCTTGTCGAAGCCCAGA); |
| SEQ ID NO: 40 | |
| | (AGACCTTGTCGAAGCCCA); |
| SEQ ID NO: 44 | |
| | (GGTTGTCTCTGTCATTGAAGCCCGA); |
| SEQ ID NO: 46 | |
| | (GTCACTACAGACACCCAGTC); |
| SEQ ID NO: 47 | |
| | (GGTCACTACAGACACCCAGT); |
| SEQ ID NO: 48 | |
| | (ACCGTCAACACTGTCCCACA); |
| SEQ ID NO: 50 | |
| | (GTACCGTCAACACTGTCCCA); |
| SEQ ID NO: 51 | |
| | (ACGTACCGTCAACACTGTCC); |
| SEQ ID NO: 52 | |
| | (GACGTACCGTCAACACTGTC); |
| | and |
| SEQ ID NO: 53 | |
| | (GGACGTACCGTCAACACTGT). |

In an embodiment, the antisense oligomers, or pharmaceutically acceptable salts thereof, disclosed herein, including antisense oligomers, or pharmaceutically acceptable salts thereof, of Formula (II), have a targeting sequence complementary to a target region within the 15^{th} nucleotide of intron 4, as measured from the 5′ end of exon 5 and the 10^{th} nucleotide of exon 5, as measured from 5′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 159. In a further embodiment, the target region is selected from H5A(−15+5), H5A(−14+6), H5A(−11+9), and H5A(−10+10). In still a further embodiment, the antisense oligomers, or pharmaceutically acceptable salts thereof, disclosed herein, including antisense oligomers, or pharmaceutically acceptable salts thereof, of Formula (II), have a targeting sequence comprising or consisting of a sequence selected from SEQ ID NOs: 30-33.

In an embodiment, the target region is H5A(−15+5). In another embodiment, the targeting sequence comprises SEQ ID NO: 30.

In an embodiment, the target region is H5A(−14+6). In another embodiment, the target region is H5A(−15+5). In certain embodiments the targeting sequence comprises SEQ ID NO: 31.

In an embodiment, the target region is H5A(−11+9). In another embodiment, the target region is H5A(−11+9). In some embodiments, the targeting sequence comprises SEQ ID NO: 32.

In an embodiment, the target region is H5A(−10+10). In another embodiment, the targeting sequence comprises SEQ ID NO: 33.

In an embodiment, antisense oligomers, or pharmaceutically acceptable salts thereof, disclosed herein, including antisense oligomers, or pharmaceutically acceptable salts thereof, of Formula (II), have a targeting sequence complementary to the target region H5A(+51+75). In a further embodiment, antisense oligomers, or pharmaceutically acceptable salts thereof, disclosed herein, including antisense oligomers, or pharmaceutically acceptable salts thereof, of Formula (II), the targeting sequence comprises SEQ ID NO: 35.

In an embodiment, antisense oligomers, or pharmaceutically acceptable salts thereof, of the disclosed herein, including antisense oligomers, or pharmaceutically acceptable salts thereof, of Formula (II), have a targeting sequence complementary to a target region within the 76^{th} nucleotide to the 100^{th} nucleotide, as measured from the 5′ end of exon 5, of the human UMOD gene pre-mRNA. In certain embodiments, the antisense oligomers, or pharmaceutically acceptable salts thereof, of Formula (II), have a targeting sequence complementary to a target region comprised in SEQ ID NO: 37. In a further embodiment, the target region is selected from H5A(+76+100), H5A(+78+95), H5A(+80+97), and H5A(+82+99). In one embodiment, the target region is H5A(+76+100). In another embodiment, the targeting sequence comprises SEQ ID NO: 37.

In one embodiment, the target region is H5A(+78+95). In another embodiment, the antisense oligomer, or a pharmaceutically acceptable salt thereof, has a targeting sequence comprising SEQ ID NO: 38.

In one embodiment, the target region is H5A(+80+97). In another embodiment, the antisense oligomer, or a pharmaceutically acceptable salt thereof, has a targeting sequence comprising SEQ ID NO: 39.

In one embodiment, the target region is H5A(+82+99). In another embodiment, the antisense oligomer, or a pharmaceutically acceptable salt thereof, has a targeting sequence comprising SEQ ID NO: 40.

In an embodiment, antisense oligomers, or pharmaceutically acceptable salts thereof, disclosed herein, including antisense oligomers, or pharmaceutically acceptable salts thereof, of Formula (II), have a targeting sequence complementary to a target region within the 18^{th} nucleotide of exon 5 measured from 3′ end of exon 5 to the 8^{th} nucleotide of intron 5 measured from 3′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 160. In a further embodiment, the target region is selected from H5D(+18-2), H5D(+16-4), H5D(+14-6), H5D(+13-7), and H5D(+12-8). In still a further embodiment, antisense oligomers, or pharmaceutically acceptable salts thereof, disclosed herein, including antisense oligomers, or pharmaceutically acceptable salts thereof, of Formula (II), have a targeting sequence comprising a sequence selected from SEQ ID NOs: 48 and 50-53.

In another embodiment, the target region is an intron/exon junction or an exon internal region of exon 6. In certain embodiments, the target region is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D(+24-1), H6D(+15-5), and H6D(+14-6). In certain embodiments, the antisense oligomer of Formula (II), or a pharmaceutically acceptable salt thereof, has a targeting sequence comprising a sequence selected from:

| SEQ ID NO: 57 | |
|---|---|
| | (TCATCTGCCAGGTAGAGGGTGTTGC); |
| SEQ ID NO: 58 | |
| | (GGTCACGGATGATGATCTCA); |
| SEQ ID NO: 59 | |
| | (AGGTCACGGATGATGATCTC); |
| SEQ ID NO: 61 | |
| | (TTGATGTTGAGGTCACGGAT); |
| SEQ ID NO: 62 | |
| | (TTTGATGTTGAGGTCACGGA); |
| SEQ ID NO: 64 | |
| | (GGTAGGAGCATGCAAAGTTGATTTT); |
| SEQ ID NO: 66 | |
| | (TTCAGGCTGACTTTCATGTCCAGGG); |
| SEQ ID NO: 67 | |
| | (GCTGACTTTCATGTCCAGGG); |
| SEQ ID NO: 68 | |
| | (GGTCTTCAGGCTGACTTTCA); |
| SEQ ID NO: 69 | |
| | (CGGTCTTCAGGCTGACTTTC); |
| SEQ ID NO: 70 | |
| | (GCGGTCTTCAGGCTGACTTT); |
| SEQ ID NO: 71 | |
| | (GGCGGTCTTCAGGCTGACTT); |
| SEQ ID NO: 72 | |
| | (CTGTAGGGCGGTCTTCAGGC); |
| SEQ ID NO: 73 | |
| | (GCTGTAGGGCGGTCTTCAGG); |
| SEQ ID NO: 74 | |
| | (GGCTGTAGGGCGGTCTTCAG); |
| SEQ ID NO: 75 | |
| | (TGGCTGTAGGGCGGTCTTCA); |
| SEQ ID NO: 76 | |
| | (TTGGCTGTAGGGCGGTCTTC); |
| SEQ ID NO: 77 | |
| | (ATTGGCTGTAGGGCGGTCTT); |
| SEQ ID NO: 78 | |
| | (CTGACCATTGGCTGTAGGGC); |
| SEQ ID NO: 79 | |
| | (CCTGACCATTGGCTGTAGGGCGGTC); |
| SEQ ID NO: 80 | |
| | (CACACCTGACCATTGGCTGT); |
| | and |
| SEQ ID NO: 81 | |
| | (CCACACCTGACCATTGGCTG). |

In an embodiment, antisense oligomers, or pharmaceutically acceptable salts thereof, disclosed herein, including antisense oligomers, or pharmaceutically acceptable salts thereof, of Formula (II), have a targeting sequence complementary to the target region H6A(+26+50). In a further embodiment, antisense oligomers, or pharmaceutically acceptable salts thereof, disclosed herein, including antisense oligomers, or pharmaceutically acceptable salts thereof, of Formula (II), the targeting sequence comprises SEQ ID NO: 57.

In an embodiment, antisense oligomers, or pharmaceutically acceptable salts thereof, disclosed herein, including antisense oligomers, or pharmaceutically acceptable salts thereof, of Formula (II), have a targeting sequence complementary to a target region within the 48^{th} nucleotide to the 78^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 161. In a further embodiment, the target region is selected from H6A(+48+67), H6A(+49+68), H6A(+58+77), and H6A(+59+78). In still a further embodiment, the antisense oligomer, or a pharmaceutically acceptable salt thereof, has a targeting sequence comprising a sequence selected from SEQ ID NOs: 58, 59, 61, and 62.

In another embodiment, the antisense oligomers disclosed herein, or pharmaceutically acceptable salts thereof, including antisense oligomers, or pharmaceutically acceptable salts thereof, of Formula (II), have a targeting sequence complementary to a target region within the 58^{th} nucleotide to the 78^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 162. In certain embodiments, the target region is selected from H6A(+58+77) and H6A(+59+78). In one embodiment, the target region is H6A(+58+77). In one embodiment, the antisense oligomer, or a pharmaceutically acceptable salt thereof, has a targeting sequence comprising SEQ ID NO: 61. In another embodiment, the target region is H6A(+59+78). In one embodiment, the antisense oligomer, or a pharmaceutically acceptable salt thereof, has a targeting sequence comprising SEQ ID NO: 62.

In an embodiment, the antisense oligomers disclosed herein, or pharmaceutically acceptable salts thereof, including antisense oligomers, or pharmaceutically acceptable salts thereof, of Formula (II), have a targeting sequence complementary to the target region H6A(+76+100). In a further embodiment, the antisense oligomer, or a pharmaceutically acceptable salt thereof, has a targeting sequence complementary to the target region H6A(+26+50). In another embodiment, the targeting sequence comprises SEQ ID NO: 64.

In an embodiment, the antisense oligomers disclosed herein, or pharmaceutically acceptable salts thereof, including antisense oligomers, or pharmaceutically acceptable salts thereof, of Formula (II), have a targeting sequence complementary to a target region within the 101^{st} nucleotide to the 132^{nd} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 163. In a further embodiment, the target region is selected from H6A(+101+125), H6A(+101+120), H6A(+110+129), H6A(+111+130), H6A(+112+131), and H6A(+113+132). In still a further embodiment, the antisense oligomers disclosed herein, or pharmaceutically acceptable salts thereof, including antisense oligomers, or pharmaceutically acceptable salts thereof, of Formula (II), have a targeting sequence comprising a sequence selected from SEQ ID NOs: 66-71.

In another embodiment, the antisense oligomers disclosed herein, or pharmaceutically acceptable salt thereof, including antisense oligomers, or pharmaceutically acceptable salts thereof, of Formula (II), have a targeting sequence complementary to a target region within the 111^{th} nucleotide to the 132^{nd} nucleotide measured from the 5′ end of exon 6, of the human UMOD gene pre-mRNA. In certain embodiments, the antisense oligomers, or pharmaceutically acceptable salts thereof, of Formula (II), have a targeting sequence complementary to a target region comprised in SEQ ID NO: 164. In certain embodiments, the target region is selected from H6A(+111+130), H6A(+112+131), and H6A(+113+132). In one embodiment, the target region is H6A(+111+130). In one embodiment, the antisense oligomer, or a pharmaceutically acceptable salt thereof, has a targeting sequence comprising SEQ ID NO: 69. In another embodiment, the target region is H6A(+112+131). In certain embodiments and the antisense oligomer, or a pharmaceutically acceptable salt thereof, has a targeting sequence comprising SEQ ID NO: 70. In yet another embodiment, the target region is H6A(+113+133). In certain embodiments, the antisense oligomer, or a pharmaceutically acceptable salt thereof, has a targeting sequence comprising SEQ ID NO: 71.

In an embodiment, the antisense oligomers disclosed herein, or pharmaceutically acceptable salts thereof, including antisense oligomers, or pharmaceutically acceptable salts thereof, of Formula (II), have a targeting sequence complementary to a target region within the 119^{th} nucleotide to the 149^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 165. In another embodiment, the target region is selected from H6A(+119+138), H6A(+120+139), H6A(+121+140), H6A(+122+141), H6A(+123+142), H6A(+124+143), and H6A(+130+149). In yet another embodiment, the antisense oligomer, or a pharmaceutically acceptable salt thereof, has a targeting sequence comprising a sequence selected from SEQ ID NOs: 72-78.

In a further embodiment, the antisense oligomers disclosed herein, or pharmaceutically acceptable salts thereof, including antisense oligomers, or pharmaceutically acceptable salts thereof, of Formula (II), have a targeting sequence complementary to a target region within the 119^{th} nucleotide to the 141^{st} nucleotide measured from the 5′ end of exon 6., of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 166. In an embodiment, the target region is selected from H6A(+119+138), H6A(+120+139), H6A(+121+140), and H6A(+122+141). In another embodiment, the antisense oligomer, or a pharmaceutically acceptable salt thereof, has a targeting sequence comprising a sequence selected from SEQ ID NOs: 72-75.

In still a further embodiment, the antisense oligomers disclosed herein, or pharmaceutically acceptable salts thereof, including antisense oligomers, or pharmaceutically acceptable salts thereof, of Formula (II), have a targeting sequence complementary to a target region within the 120^{th} nucleotide to the 141^{st} nucleotide measured from the 5′ end of exon 6, of the human UMOD gene pre-mRNA. In certain embodiments, the antisense oligomers, or pharmaceutically acceptable salts thereof, of Formula (II), have a targeting sequence complementary to a target region comprised in SEQ ID NO: 167. In some embodiments, the target region is H6A(+120+139), H6A(+121+140), or H6A(+122+141). In one embodiment, the target region is H6A(+120+139). In another embodiment, the antisense oligomer, or a pharmaceutically acceptable salt thereof, has a targeting sequence comprising SEQ ID NO: 73. In another embodiment, the target region is H6A(+121+140). In certain embodiments, the antisense oligomer, or a pharmaceutically acceptable salt thereof, has a targeting sequence comprising SEQ ID NO: 74. In yet another embodiment, the target region is H6A(+122+141). In certain embodiments, the antisense oligomer, or a pharmaceutically acceptable salt thereof, has a targeting sequence comprising SEQ ID NO: 75.

In still a further embodiment, the antisense oligomers disclosed herein, or pharmaceutically acceptable salts thereof, including antisense oligomers, or pharmaceutically acceptable salts thereof, of Formula (II), have a targeting sequence complementary to a target region within the 123^{rd} nucleotide to the 149^{th} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 168. In an embodiment, the target region is selected from H6A(+123+142), H6A(+124+143), and H6A(+130+149). In another embodiment, the antisense oligomer, or a pharmaceutically acceptable salt thereof, has a targeting sequence comprising a sequence selected from SEQ ID NOs: 76-78.

In an embodiment, the antisense oligomers disclosed herein, or pharmaceutically acceptable salts thereof, including antisense oligomers, or pharmaceutically acceptable salts thereof, of Formula (II), have a targeting sequence complementary to a target region within the 24^{th} nucleotide of exon 6 measured from 3′ end of exon 6 to the 6^{th} nucleotide of intron 6 measured from 3′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 169. In a further embodiment, the target region is selected from H6D(+24-1), H6D(+15-5), and H6D(+14-6). In still a further embodiment, the antisense oligomer, or a pharmaceutically acceptable salt thereof, has a targeting sequence comprising a sequence selected from SEQ ID NOs: 79-81.

In a further embodiment, the antisense oligomers disclosed herein, or pharmaceutically acceptable salts thereof, including antisense oligomers, or pharmaceutically acceptable salts thereof, of Formula (II), have a targeting sequence complementary to a target region within the 15^{th} nucleotide of exon 6 measured from 3′ end of exon 6 to the 6^{th} nucleotide of intron 6 measured from 3′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (II), have a targeting sequence complementary to a target region comprised in SEQ ID NO: 170. In some embodiments, the target region is H6D(+15-5), or H6D(+14-6). In one embodiment, the target region is H6D(+15-5). In certain embodiments, the antisense oligomer, or a pharmaceutically acceptable salt thereof, has a targeting sequence comprising SEQ ID NO: 80. In another embodiment, the target region is H6D(+14-6). In certain embodiments, the antisense oligomer, or a pharmaceutically acceptable salt thereof, has a targeting sequence comprising SEQ ID NO: 81.

In yet another embodiment, the target region is an intron/exon junction or an exon internal region of exon 8. In certain embodiments, the target region is selected from H8A(−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D(+12-13). In certain embodiments, the antisense oligomer of Formula (II), or a pharmaceutically acceptable salt thereof, has a targeting sequence comprising a sequence selected from:

| SEQ ID NO: 82 | |
|---|---|
| | (TTGAGTCTCTAGTGTGTGGGCATCT); |
| SEQ ID NO: 84 | |
| | (TGGACGGAAAATCGGCCCTGGGAGG); |
| SEQ ID NO: 85 | |
| | (CATCTGGACGGAAAATCGGC); |
| SEQ ID NO: 86 | |
| | (ACATCTGGACGGAAAATCGG); |
| SEQ ID NO: 89 | |
| | (AACCGGAACATCTGGACGGA); |
| SEQ ID NO: 90 | |
| | (AAACCGGAACATCTGGACGG); |
| SEQ ID NO: 91 | |
| | (CAAACCGGAACATCTGGACG); |
| SEQ ID NO: 92 | |
| | (TTCCAGCAAACCGGAACATC); |
| SEQ ID NO: 93 | |
| | (ATAGTTTCCAGCAAACCGGAACATC); |
| SEQ ID NO: 94 | |
| | (TTTCCAGCAAACCGGAACAT); |
| SEQ ID NO: 95 | |
| | (GTTTCCAGCAAACCGGAACA); |
| SEQ ID NO: 96 | |
| | (AGTTTCCAGCAAACCGGAAC); |
| SEQ ID NO: 98 | |
| | (ATAGTTTCCAGCAAACCGGA); |
| SEQ ID NO: 99 | |
| | (CATAGTTTCCAGCAAACCGG); |
| SEQ ID NO: 100 | |
| | (TCATAGTTTCCAGCAAACCG); |
| SEQ ID NO: 101 | |
| | (AGGTCATAGTTTCCAGCAAA); |
| SEQ ID NO: 102 | |
| | (GGTAGACTAGGTCATAGTTT); |
| SEQ ID NO: 103 | |
| | (AGGTAGACTAGGTCATAGTT); |
| SEQ ID NO: 104 | |
| | (CAGGTAGACTAGGTCATAGT); |
| SEQ ID NO: 105 | |
| | (CTTCACAGTGCAGGTAGACTAGGTC); |
| SEQ ID NO: 106 | |
| | (ACAGTGCAGGTAGACTAGGT); |
| SEQ ID NO: 107 | |
| | (CACAGTGCAGGTAGACTAGG); |
| SEQ ID NO: 108 | |
| | (TCACAGTGCAGGTAGACTAG); |
| SEQ ID NO: 109 | |
| | (TTCACAGTGCAGGTAGACTA); |
| SEQ ID NO: 110 | |
| | (GTCACAGAGATAGACTTCAC); |
| SEQ ID NO: 111 | |
| | (TGTCACAGAGATAGACTTCA); |
| SEQ ID NO: 112 | |
| | (GTGTCACAGAGATAGACTTC); |
| SEQ ID NO: 113 | |
| | (TCATTCATGGTGTCACAGAGATAGA); |
| SEQ ID NO: 114 | |
| | (TTCATGGTGTCACAGAGATA); |
| SEQ ID NO: 115 | |
| | (ATTCATGGTGTCACAGAGAT); |
| | and |
| SEQ ID NO: 120 | |
| | (GAGTCAACTCACAGGCTTGCACTTT). |

In an embodiment, the antisense oligomer, or a pharmaceutically acceptable salt thereof, has a targeting sequence complementary to the target region H8A(−2+23). In a further embodiment, the antisense oligomer, or a pharmaceutically acceptable salt thereof, has a targeting sequence comprising SEQ ID NO: 82. In still a further embodiment, the antisense oligomer, or a pharmaceutically acceptable salt thereof, has a targeting sequence complementary to the target region H8A(−2+23), wherein the targeting sequence comprises SEQ ID NO: 82.

In some embodiments, the antisense oligomer, or a pharmaceutically acceptable salt thereof, has a targeting sequence complementary to a target region within the 51^{st} nucleotide to the 80^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 171. In a further embodiment, the target region is selected from H8A(+51+75), H8A(+60+79), and H8A(+61+80). In one embodiment, the antisense oligomer, or a pharmaceutically acceptable salt thereof, has a targeting sequence comprising a sequence selected from SEQ ID NOs: 84-86.

In an embodiment, the antisense oligomer, or a pharmaceutically acceptable salt thereof, has a targeting sequence complementary to a target region within the 68^{th} nucleotide to the 100^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 172. In another embodiment, the target region is selected from H8A(+68+87), H8A(+69+88), H8A(+70+89), H8A(+76+95), H8A(+76+100), H8A(+77+96), H8A(+78+97), and H8A(+79+98). In yet another embodiment, the antisense oligomer, or a pharmaceutically acceptable salt thereof, has a targeting sequence comprising a sequence selected from SEQ ID NOs: 89-96.

In a further embodiment, the antisense oligomer, or a pharmaceutically acceptable salt thereof, has a targeting sequence complementary to a target region within the 76^{th} nucleotide to the 100^{th} nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 173. In an embodiment, the target region is selected from H8A(+76+95), H8A(+77+96), H8A(+78+97), and H8A(+79+98). In another embodiment, the antisense oligomer, or a pharmaceutically acceptable salt thereof, has a targeting sequence comprising a sequence selected from SEQ ID NOs: 92 and 94-96.

In an embodiment, the antisense oligomer, or a pharmaceutically acceptable salt thereof, has a targeting sequence complementary to a target region within the 81^{st} nucleotide to the 105^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 174. In a further embodiment, the target region is selected from H8A(+81+100), H8A(+82+101), H8A(+83+102), and H8A(+86+105). In still a further embodiment, the antisense oligomer, or a pharmaceutically acceptable salt thereof, has a targeting sequence comprising a sequence selected from SEQ ID NOs: 98-101.

In an embodiment, the antisense oligomer, or a pharmaceutically acceptable salt thereof, has a targeting sequence complementary to a target region within the 94^{th} nucleotide to the 124^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 175. In a further embodiment, the target region is selected from H8A(+94+113), H8A(+95+114), H8A(+96+115), H8A(+101+125), H8A(+102+121), H8A(+103+122), H8A(+104+123), and H8A(+105+124). In still a further embodiment, the antisense oligomer, or a pharmaceutically acceptable salt thereof, has a targeting sequence comprising a sequence selected from SEQ ID NOs: 102-109.

In an embodiment, the antisense oligomer, or a pharmaceutically acceptable salt thereof, has a targeting sequence complementary to a target region within the 120^{th} nucleotide to the 148^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 176. In a further embodiment, the target region is selected from H8A(+120+139), H8A(+121+140), H8A(+122+141), H8A(+126+150), H8A(+128+147), and H8A(+129+148). In still a further embodiment, the antisense oligomer, or a pharmaceutically acceptable salt thereof, has a targeting sequence comprising a sequence selected from SEQ ID NOs: 110-115.

In a further embodiment, the antisense oligomer, or a pharmaceutically acceptable salt thereof, has a targeting sequence complementary to a target region within the 126^{th} nucleotide to the 148^{th} nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 177. In certain embodiments, the target region is selected from H8A(+126+150), H8A(+128+147), and H8A(+129+148). In a further embodiment, the target region is selected from H8A(+126+150), H8A(+128+147), and H8A(+129+148). In some embodiments, the antisense oligomer, or a pharmaceutically acceptable salt thereof, has a targeting sequence comprising a sequence selected from SEQ ID NOs: 113-115. In one embodiment, the target region is H8A(+128+147). In some embodiments, the antisense oligomer, or a pharmaceutically acceptable salt thereof, has a targeting sequence comprising SEQ ID NO: 114.

In an embodiment, the antisense oligomer, or a pharmaceutically acceptable salt thereof, has a targeting sequence complementary to the target region H8D(+12-13). In another embodiment, the target region is H8D(+12-13). In some embodiments, the antisense oligomer, or a pharmaceutically acceptable salt thereof, has a targeting sequence comprising SEQ ID NO: 120.

In still another embodiment, the target region is an intron/exon junction or an exon internal region of exon 9. In certain embodiments, the target region is selected from H9A(−5+20), H9A(+1+25), H9A(+51+75), and H9D(+7-18). In certain embodiments, the antisense oligomer of Formula (II), or a pharmaceutically acceptable salt thereof, has a targeting sequence comprising a sequence selected from:

| SEQ ID NO: 121 | |
|---|---|
| | (AATCTGGTCCCAGAGCAGGTCTACA); |
| SEQ ID NO: 122 | |
| | (TTCGGAATCTGGTCCCAGAGCAGGT); |
| SEQ ID NO: 123 | |
| | (TGTGATGGGACCCAAGTTCAGGACA); |
| | and |
| SEQ ID NO: 124 | |
| | (AGCGAGTGGCTCTCTTACCTTTCCG). |

In an embodiment, the antisense oligomer, or a pharmaceutically acceptable salt thereof, has a targeting sequence complementary to a target region within the 5^{th} nucleotide of intron 8 measured from the 5′ end of exon 9 and the 25^{th} nucleotide of exon 9 measured from the 5′ end of exon 9 of the human UMOD gene pre-mRNA. In certain embodiments, the antisense oligomer, or a pharmaceutically acceptable salt thereof, has a targeting sequence complementary to a target region comprised in SEQ ID NO: 178. In a further embodiment, the target region is H9A(−5+20) or H9A(+1+25). In still a further embodiment, the antisense oligomer, or a pharmaceutically acceptable salt thereof, has a targeting sequence comprising a sequence selected from SEQ ID NOs: 121 and 122.

In an embodiment, the antisense oligomer, or a pharmaceutically acceptable salt thereof, has a target sequence complementary to the target region H9A(+51+75). In some embodiments, the target region is H9A(+51+75). In some embodiments, the antisense oligomer, or a pharmaceutically acceptable salt thereof, has a targeting sequence comprising SEQ ID NO: 123.

In another embodiment, the antisense oligomer, or a pharmaceutically acceptable salt thereof, has a target sequence complementary to the target region H9D(+7-18). In some embodiments, the target region is H9D(+7-18). In some embodiments, the antisense oligomer, or a pharmaceutically acceptable salt thereof, has a targeting sequence comprising SEQ ID NO: 124.

In an embodiment, the antisense oligomer of Formula (II), or a pharmaceutically acceptable salt thereof, has a targeting sequence complementary to a region within an intron/exon junction or an exon internal junction of the human UMOD gene pre-mRNA, wherein the targeting sequence comprises a sequence selected from:

| SEQ ID NO: 32 | |
|---|---|
| | (GGGAGATATCTGAAACAGGT); |
| SEQ ID NO: 37 | |
| | (AAGACCTTGTCGAAGCCCAGACTCT); |
| SEQ ID NO: 62 | |
| | (TTTGATGTTGAGGTCACGGA); |
| SEQ ID NO: 70 | |
| | (GCGGTCTTCAGGCTGACTTT); |
| SEQ ID NO: 74 | |
| | (GGCTGTAGGGCGGTCTTCAG); |
| SEQ ID NO: 81 | |
| | (CCACACCTGACCATTGGCTG); |
| SEQ ID NO: 114 | |
| | (TTCATGGTGTCACAGAGATA); |
| | and |
| SEQ ID NO: 120 | |
| | (GAGTCAACTCACAGGCTTGCACTTT). |

### Formula (III) and (IV)

In some embodiments, the antisense oligomer of Formula (I) is an antisense oligomer of Formula (III):

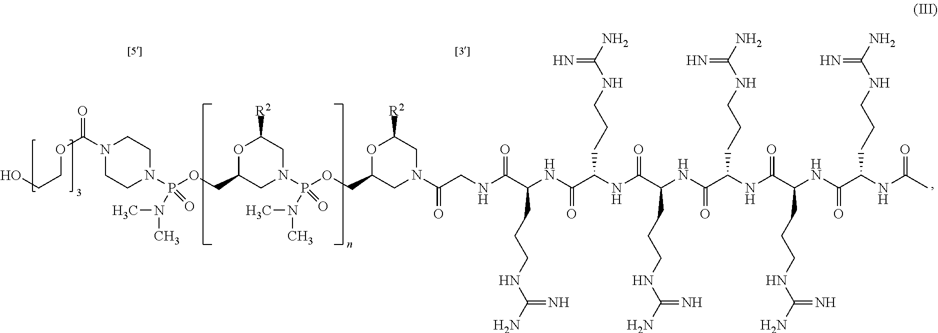

or a pharmaceutically acceptable salt thereof,
wherein:
each R² is independently selected from a naturally or non-naturally occurring nucleobase, which, when taken together, forms a targeting sequence of 13 to 30 bases in length that is complementary to a target region within a pre-mRNA of the human uromodulin (UMOD) gene represented by SEQ ID NO: 1, wherein the target region is an intron/exon junction or an exon internal region of the human UMOD gene pre-mRNA; and
n is 11-28.

In some embodiments, the antisense oligomer of Formula (III) has the structure of Formula (IV):

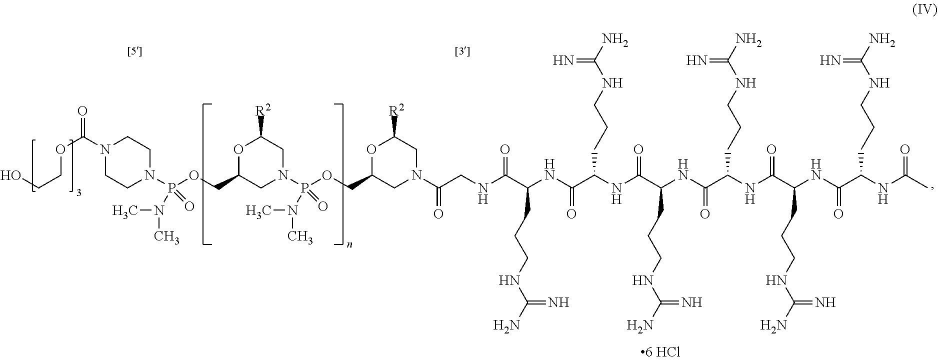

wherein:
each R² is independently selected from a naturally or non-naturally occurring nucleobase, which, when taken together, forms a targeting sequence of 13 to 30 bases in length that is complementary to a target region within a pre-mRNA of the human uromodulin (UMOD) gene represented by SEQ ID NO: 1, wherein the target region is an intron/exon junction or an exon internal region of the human UMOD gene pre-mRNA; and n is 11-28.

In an embodiment of Formula (III) and/or (IV), n is an integer from 11 to 28. In an embodiment of Formula (III) and/or (IV), n is at least 11. In an embodiment of Formula (III) and/or (IV), n is up to 28. In an embodiment of Formula (III) and/or (IV), n is 11-28. In an embodiment of Formula (III) and/or (IV), n is 11-27. In an embodiment of Formula (III) and/or (IV), n is 11-26. In an embodiment of Formula (III) and/or (IV), n is 11-25. In an embodiment of Formula (III) and/or (IV), n is 11-24. In an embodiment of Formula (III) and/or (IV), n is 11-23. In an embodiment of Formula (III) and/or (IV), n is 11-22. In an embodiment of Formula (III) and/or (IV), n is 11-21. In an embodiment of Formula (III) and/or (IV), n is 11-20. In an embodiment of Formula (III) and/or (IV), n is 11-19. In an embodiment of Formula (III) and/or (IV), n is 11-18. In an embodiment of Formula (III) and/or (IV), n is 11-17. In an embodiment of Formula (III) and/or (IV), n is 11-16. In an embodiment of Formula (III) and/or (IV), n is 11-15. In an embodiment of Formula (III) and/or (IV), n is 11-14. In an embodiment of Formula (III) and/or (IV), n is 11-13. In an embodiment of Formula (III) and/or (IV), n is 11-12. In an embodiment of Formula (III) and/or (IV), n is 12-28. In an embodiment of Formula (III) and/or (IV), n is 12-27. In an embodiment of Formula (III) and/or (IV), n is 12-26. In an embodiment of Formula (III) and/or (IV), n is 12-25. In an embodiment of Formula (III) and/or (IV), n is 12-24. In an embodiment of Formula (III) and/or (IV), n is 12-23. In an embodiment of Formula (III) and/or (IV), n is 12-22. In an embodiment of Formula (III) and/or (IV), n is 12-21. In an embodiment of Formula (III) and/or (IV), n is 12-20. In an embodiment of Formula (III) and/or (IV), n is 12-19. In an embodiment of Formula (III) and/or (IV), n is 12-18. In an embodiment of Formula (III) and/or (IV), n is 12-17. In an embodiment of Formula (III) and/or (IV), n is 12-16. In an embodiment of Formula (III) and/or (IV), n is 12-15. In an embodiment of Formula (III) and/or (IV), n is 12-14. In an embodiment of Formula (III) and/or (IV), n is 12-13. In an embodiment of Formula (III) and/or (IV), n is 13-28. In an embodiment of Formula (III) and/or (IV), n is 13-27. In an embodiment of Formula (III) and/or (IV), n is 13-26. In an embodiment of Formula (III) and/or (IV), n is 13-25. In an embodiment of Formula (III) and/or (IV), n is 13-24. In an embodiment of Formula (III) and/or (IV), n is 13-23. In an embodiment of Formula (III) and/or (IV), n is 13-22. In an embodiment of Formula (III) and/or (IV), n is 13-21. In an embodiment of Formula (III) and/or (IV), n is 13-20. In an embodiment of Formula (III) and/or (IV), n is 13-19. In an embodiment of Formula (III) and/or (IV), n is 13-18. In an embodiment of Formula (III) and/or (IV), n is 13-17. In an embodiment of Formula (III) and/or (IV), n is 13-16. In an embodiment of Formula (III) and/or (IV), n is 13-15. In an embodiment of Formula (III) and/or (IV), n is 13-14. In an embodiment of Formula (III) and/or (IV), n is 14-28. In an embodiment of Formula (III) and/or (IV), n is 14-27. In an embodiment of Formula (III) and/or (IV), n is 14-26. In an embodiment of Formula (III) and/or (IV), n is 14-25. In an embodiment of Formula (III) and/or (IV), n is 14-24. In an embodiment of Formula (III) and/or (IV), n is 14-23. In an embodiment of Formula (III) and/or (IV), n is 14-22. In an embodiment of Formula (III) and/or (IV), n is 14-21. In an embodiment of Formula (III) and/or (IV), n is 14-20. In an embodiment of Formula (III) and/or (IV), n is 14-19. In an embodiment of Formula (III) and/or (IV), n is 14-18. In an embodiment of Formula (III) and/or (IV), n is 14-17. In an embodiment of Formula (III) and/or (IV), n is 14-16. In an embodiment of Formula (III) and/or (IV), n is 14-15. In an embodiment of Formula (III) and/or (IV), n is 15-28. In an embodiment of Formula (III) and/or (IV), n is 15-27. In an embodiment of Formula (III) and/or (IV), n is 15-26. In an embodiment of Formula (III) and/or (IV), n is 15-25. In an embodiment of Formula (III) and/or (IV), n is 15-24. In an embodiment of Formula (III) and/or (IV), n is 15-23. In an embodiment of Formula (III) and/or (IV), n is 15-22. In an embodiment of Formula (III) and/or (IV), n is 15-21. In an embodiment of Formula (III) and/or (IV), n is 15-20. In an embodiment of Formula (III) and/or (IV), n is 15-19. In an embodiment of Formula (III) and/or (IV), n is 15-18. In an embodiment of Formula (III) and/or (IV), n is 15-17. In an embodiment of Formula (III) and/or (IV), n is 15-16. In an embodiment of Formula (III) and/or (IV), n is 16-28. In an embodiment of Formula (III) and/or (IV), n is 16-27. In an embodiment of Formula (III) and/or (IV), n is 16-26. In an embodiment of Formula (III) and/or (IV), n is 16-25. In an embodiment of Formula (III) and/or (IV), n is 16-24. In an embodiment of Formula (III) and/or (IV), n is 16-23. In an embodiment of Formula (III) and/or (IV), n is 16-22. In an embodiment of Formula (III) and/or (IV), n is 16-21. In an embodiment of Formula (III) and/or (IV), n is 16-20. In an embodiment of Formula (III) and/or (IV), n is 16-19. In an embodiment of Formula (III) and/or (IV), n is 16-18. In an embodiment of Formula (III) and/or (IV), n is 16-17. In an embodiment of Formula (III) and/or (IV), n is 17-28. In an embodiment of Formula (III) and/or (IV), n is 17-27. In an embodiment of Formula (III) and/or (IV), n is 17-26. In an embodiment of Formula (III) and/or (IV), n is 17-25. In an embodiment of Formula (III) and/or (IV), n is 17-24. In an embodiment of Formula (III) and/or (IV), n is 17-23. In an embodiment of Formula (III) and/or (IV), n is 17-22. In an embodiment of Formula (III) and/or (IV), n is 17-21. In an embodiment of Formula (III) and/or (IV), n is 17-20. In an embodiment of Formula (III) and/or (IV), n is 17-19. In an embodiment of Formula (III) and/or (IV), n is 17-18. In an embodiment of Formula (III) and/or (IV), n is 18-28. In an embodiment of Formula (III) and/or (IV), n is 18-27. In an embodiment of Formula (III) and/or (IV), n is 18-26. In an embodiment of Formula (III) and/or (IV), n is 18-25. In an embodiment of Formula (III) and/or (IV), n is 18-24. In an embodiment of Formula (III) and/or (IV), n is 18-23. In an embodiment of Formula (III) and/or (IV), n is 18-22. In an embodiment of Formula (III) and/or (IV), n is 18-21. In an embodiment of Formula (III) and/or (IV), n is 18-20. In an embodiment of Formula (III) and/or (IV), n is 18-19. In an embodiment of Formula (III) and/or (IV), n is 19-28. In an embodiment of Formula (III) and/or (IV), n is 19-27. In an embodiment of Formula (III) and/or (IV), n is 19-26. In an embodiment of Formula (III) and/or (IV), n is 19-25. In an embodiment of Formula (III) and/or (IV), n is 19-24. In an embodiment of Formula (III) and/or (IV), n is 19-23. In an embodiment of Formula (III) and/or (IV), n is 19-22. In an embodiment of Formula (III) and/or (IV), n is 19-21. In an embodiment of Formula (III) and/or (IV), n is 19-20. In an embodiment of Formula (III) and/or (IV), n is 20-28. In an embodiment of Formula (III) and/or (IV), n is 20-27. In an embodiment of Formula (III) and/or (IV), n is 20-26. In an embodiment of Formula (III) and/or (IV), n is 20-25. In an embodiment of Formula (III) and/or (IV), n is 20-24. In an embodiment of Formula (III) and/or (IV), n is 20-23. In an embodiment of Formula (III) and/or (IV), n is 20-22. In an embodiment of Formula (III) and/or (IV), n is 20-21. In an embodiment of Formula (III) and/or (IV), n is 21-28. In an embodiment of Formula (III) and/or (IV), n is 21-27. In an embodiment of Formula (III) and/or (IV), n is 21-26. In an embodiment of Formula (III) and/or (IV), n is 21-25. In an embodiment of Formula (III) and/or (IV), n is 21-24. In an embodiment of Formula (III) and/or (IV), n is 21-23. In an embodiment of Formula (III) and/or (IV), n is 21-22. In an embodiment of Formula (III) and/or (IV), n is 22-28. In an embodiment of Formula (III) and/or (IV), n is 22-27. In an embodiment of Formula (III) and/or (IV), n is 22-26. In an embodiment of Formula (III) and/or (IV), n is 22-25. In an embodiment of Formula (III) and/or (IV), n is 22-24. In an embodiment of Formula (III) and/or (IV), n is 22-23. In an embodiment of Formula (III) and/or (IV), n is 23-28. In an embodiment of Formula (III) and/or (IV), n is 23-27. In an embodiment of Formula (III) and/or (IV), n is 23-26. In an embodiment of Formula (III) and/or (IV), n is 23-25. In an embodiment of Formula (III) and/or (IV), n is 23-24. In an embodiment of Formula (III) and/or (IV), n is 24-28. In an embodiment of Formula (III) and/or (IV), n is 24-27. In an embodiment of Formula (III) and/or (IV), n is 24-26. In an embodiment of Formula (III) and/or (IV), n is 24-25. In an embodiment of Formula (III) and/or (IV), n is 25-28. In an embodiment of Formula (III) and/or (IV), n is 25-27. In an embodiment of Formula (III) and/or (IV), n is 25-26. In another embodiment of Formula (III) and/or (IV), n is 26-30. In an embodiment of Formula (III) and/or (IV), n is 26-29. In an embodiment of Formula (III) and/or (IV), n is 26-28. In an embodiment of Formula (III) and/or (IV), n is 26-27. In an embodiment of Formula (III) and/or (IV), n is 27-28.

In an embodiment of Formula (III) and/or (IV), n is 11. In an embodiment of Formula (III) and/or (IV), n is 12. In an embodiment of Formula (III) and/or (IV), n is 13. In an embodiment of Formula (III) and/or (IV), n is 14. In an embodiment of Formula (III) and/or (IV), n is 15. In an embodiment of Formula (III) and/or (IV), n is 16. In an embodiment of Formula (III) and/or (IV), n is 17. In an embodiment of Formula (III) and/or (IV), n is 18. In an embodiment of Formula (III) and/or (IV), n is 19. In an embodiment of Formula (III) and/or (IV), n is 20. In an embodiment of Formula (III) and/or (IV), n is 21. In an embodiment of Formula (III) and/or (IV), n is 22. In an embodiment of Formula (III) and/or (IV), n is 23. In an embodiment of Formula (III) and/or (IV), n is 24. In an embodiment of Formula (III) and/or (IV), n is 25. In an embodiment of Formula (III) and/or (IV), n is 26. In an embodiment of Formula (III) and/or (IV), n is 27. In an embodiment of Formula (III) and/or (IV), n is 28.

In an embodiment of Formula (III) and/or (IV), each R² is independently selected from a naturally or non-naturally occurring nucleobase, which, when taken together, forms a targeting sequence of from 13 to 30 bases in length. In an embodiment of Formula (III) and/or (IV), each R² is independently selected from a naturally or non-naturally occurring nucleobase, which, when taken together, forms a targeting sequence at least 13 bases in length. In an embodiment of Formula (III) and/or (IV), each R² is independently selected from a naturally or non-naturally occurring nucleobase, which, when taken together, forms a targeting sequence of up to 30 bases in length.

In an embodiment of Formula (III) and/or (IV), each R² is independently selected from a naturally or non-naturally occurring nucleobase, which, when taken together, forms a targeting sequence of 13-29 bases in length. In an embodiment of Formula (III) and/or (IV), each R² is independently selected from a naturally or non-naturally occurring nucleobase, which, when taken together, forms a targeting sequence of 13-28 bases in length. In an embodiment of Formula (III) and/or (IV), each R² is independently selected from a naturally or non-naturally occurring nucleobase, which, when taken together, forms a targeting sequence of 13-27 bases in length. In an embodiment of Formula (III) and/or (IV), each R² is independently selected from a naturally or non-naturally occurring nucleobase, which, when taken together, forms a targeting sequence of 13-26 bases in length. In an embodiment of Formula (III) and/or (IV), each R² is independently selected from a naturally or non-naturally occurring nucleobase, which, when taken together, forms a targeting sequence of 13-25 bases in length. In an embodiment of Formula (III) and/or (IV), each R² is independently selected from a naturally or non-naturally occurring nucleobase, which, when taken together, forms a targeting sequence of 13-24 bases in length. In an embodiment of Formula (III) and/or (IV), each R² is independently selected from a naturally or non-naturally occurring nucleobase, which, when taken together, forms a targeting sequence of 13-23 bases in length. In an embodiment of Formula (III) and/or (IV), each R² is independently selected from a naturally or non-naturally occurring nucleobase, which, when taken together, forms a targeting sequence of 13-22 bases in length. In an embodiment of Formula (III) and/or (IV), each R² is independently selected from a naturally or non-naturally occurring nucleobase, which, when taken together, forms a targeting sequence of 13-21 bases in length. In an embodiment of Formula (III) and/or (IV), each R² is independently selected from a naturally or non-naturally occurring nucleobase, which, when taken together, forms a targeting sequence of 13-20 bases in length. In an embodiment of Formula (III) and/or (IV), each R² is independently selected from a naturally or non-naturally occurring nucleobase, which, when taken together, forms a targeting sequence of 13-19 bases in length. In an embodiment of Formula (III) and/or (IV), each R² is independently selected from a naturally or non-naturally occurring nucleobase, which, when taken together, forms a targeting sequence of 13-18 bases in length. In an embodiment of Formula (III) and/or (IV), each R² is independently selected from a naturally or non-naturally occurring nucleobase, which, when taken together, forms a targeting sequence of 13-17 bases in length. In an embodiment of Formula (III) and/or (IV), each R² is independently selected from a naturally or non-naturally occurring nucleobase, which, when taken together, forms a targeting sequence of 13-16 bases in length. In an embodiment of Formula (III) and/or (IV), each R² is independently selected from a naturally or non-naturally occurring nucleobase, which, when taken together, forms a targeting sequence of 13-15 bases in length. In an embodiment of Formula (III) and/or (IV), each R² is independently selected from a naturally or non-naturally occurring nucleobase, which, when taken together, forms a targeting sequence of 13-14 bases in length.

In another embodiment of Formula (III) and/or (IV), each R² is independently selected from a naturally or non-naturally occurring nucleobase, which, when taken together, forms a targeting sequence of 14-30 bases in length. In an embodiment of Formula (III) and/or (IV), each R² is independently selected from a naturally or non-naturally occurring nucleobase, which, when taken together, forms a targeting sequence of 14-29 bases in length. In an embodiment of Formula (III) and/or (IV), each R² is independently selected from a naturally or non-naturally occurring nucleobase, which, when taken together, forms a targeting sequence of 14-28 bases in length. In an embodiment of Formula (III) and/or (IV), each R² is independently selected from a naturally or non-naturally occurring nucleobase, which, when taken together, forms a targeting sequence of 14-27 bases in length. In an embodiment of Formula (III) and/or (IV), each R² is independently selected from a naturally or non-naturally occurring nucleobase, which, when taken together, forms a targeting sequence of 14-26 bases in length. In an embodiment of Formula (III) and/or (IV), each R² is independently selected from a naturally or non-naturally occurring nucleobase, which, when taken together, forms a targeting sequence of 14-25 bases in length. In an embodiment of Formula (III) and/or (IV), each R² is independently selected from a naturally or non-naturally occurring nucleobase, which, when taken together, forms a targeting sequence of 14-24 bases in length. In an embodiment of Formula (III) and/or (IV), each R² is independently selected from a naturally or non-naturally occurring nucleobase, which, when taken together, forms a targeting sequence of 14-23 bases in length. In an embodiment of Formula (III) and/or (IV), each R² is independently selected from a naturally or non-naturally occurring nucleobase, which, when taken together, forms a targeting sequence of 14-22 bases in length. In an embodiment of Formula (III) and/or (IV), each R² is independently selected from a naturally or non-naturally occurring nucleobase, which, when taken together, forms a targeting sequence of 14-21 bases in length. In an embodiment of Formula (III) and/or (IV), each R² is independently selected from a naturally or non-naturally occurring nucleobase, which, when taken together, forms a targeting sequence of 14-20 bases in length. In an embodiment of Formula (III) and/or (IV), each R² is independently selected from a naturally or non-naturally occurring nucleobase, which, when taken together, forms a targeting sequence of 14-19 bases in length. In an embodiment of Formula (III) and/or (IV), each R² is independently selected from a naturally or non-naturally occurring nucleobase, which, when taken together, forms a targeting sequence of 14-18 bases in length. In an embodiment of Formula (III) and/or (IV), each R² is independently selected from a naturally or non-naturally occurring nucleobase, which, when taken together, forms a targeting sequence of 14-17 bases in length. In an embodiment of Formula (III) and/or (IV), each R² is independently selected from a naturally or non-naturally occurring nucleobase, which, when taken together, forms a targeting sequence of 14-16 bases in length. In an embodiment of Formula (III) and/or (IV), each R² is independently selected from a naturally or non-naturally occurring nucleobase, which, when taken together, forms a targeting sequence of 14-15 bases in length.

In another embodiment of Formula (III) and/or (IV), each R² is independently selected from a naturally or non-naturally occurring nucleobase, which, when taken together, forms a targeting sequence of 15-30 bases in length. In an embodiment of Formula (III) and/or (IV), each R² is independently selected from a naturally or non-naturally occurring nucleobase, which, when taken together, forms a targeting sequence of 15-29 bases in length. In an embodiment of Formula (III) and/or (IV), each R² is independently selected from a naturally or non-naturally occurring nucleobase, which, when taken together, forms a targeting sequence of 15-28 bases in length. In an embodiment of Formula (III) and/or (IV), each R² is independently selected from a naturally or non-naturally occurring nucleobase, which, when taken together, forms a targeting sequence of 15-27 bases in length. In an embodiment of Formula (III) and/or (IV), each R² is independently selected from a naturally or non-naturally occurring nucleobase, which, when taken together, forms a targeting sequence of 15-26 bases in length. In an embodiment of Formula (III) and/or (IV), each R² is independently selected from a naturally or non-naturally occurring nucleobase, which, when taken together, forms a targeting sequence of 15-25 bases in length. In an embodiment of Formula (III) and/or (IV), each R² is independently selected from a naturally or non-naturally occurring nucleobase, which, when taken together, forms a targeting sequence of 15-24 bases in length. In an embodiment of Formula (III) and/or (IV), each R² is independently selected from a naturally or non-naturally occurring nucleobase, which, when taken together, forms a targeting sequence of 15-23 bases in length. In an embodiment of Formula (III) and/or (IV), each R² is independently selected from a naturally or non-naturally occurring nucleobase, which, when taken together, forms a targeting sequence of 15-22 bases in length. In an embodiment of Formula (III) and/or (IV), each R² is independently selected from a naturally or non-naturally occurring nucleobase, which, when taken together, forms a targeting sequence of 15-21 bases in length. In an embodiment of Formula (III) and/or (IV), each R² is independently selected from a naturally or non-naturally occurring nucleobase, which, when taken together, forms a targeting sequence of 15-20 bases in length. In an embodiment of Formula (III) and/or (IV), each R² is independently selected from a naturally or non-naturally occurring nucleobase, which, when taken together, forms a targeting sequence of 15-19 bases in length. In an embodiment of Formula (III) and/or (IV), each R² is independently selected from a naturally or non-naturally occurring nucleobase, which, when taken together, forms a targeting sequence of 15-18 bases in length. In an embodiment of Formula (III) and/or (IV), each R² is independently selected from a naturally or non-naturally occurring nucleobase, which, when taken together, forms a targeting sequence of 15-17 bases in length. In an embodiment of Formula (III) and/or (IV), each R² is independently selected from a naturally or non-naturally occurring nucleobase, which, when taken together, forms a targeting sequence of 15-16 bases in length.

In another embodiment of Formula (III) and/or (IV), each R² is independently selected from a naturally or non-naturally occurring nucleobase, which, when taken together, forms a targeting sequence of 16-30 bases in length. In an embodiment of Formula (III) and/or (IV), each R² is independently selected from a naturally or non-naturally occurring nucleobase, which, when taken together, forms a targeting sequence of 16-29 bases in length. In an embodiment of Formula (III) and/or (IV), each R² is independently selected from a naturally or non-naturally occurring nucleobase, which, when taken together, forms a targeting sequence of 16-28 bases in length. In an embodiment of Formula (III) and/or (IV), each R² is independently selected from a naturally or non-naturally occurring nucleobase, which, when taken together, forms a targeting sequence of 16-27 bases in length. In an embodiment of Formula (III) and/or (IV), each R² is independently selected from a naturally or non-naturally occurring nucleobase, which, when taken together, forms a targeting sequence of 16-26 bases in length. In an embodiment of Formula (III) and/or (IV), each R² is independently selected from a naturally or non-naturally occurring nucleobase, which, when taken together, forms a targeting sequence of 16-25 bases in length. In an embodiment of Formula (III) and/or (IV), each R² is independently selected from a naturally or non-naturally occurring nucleobase, which, when taken together, forms a targeting sequence of 16-24 bases in length. In an embodiment of Formula (III) and/or (IV), each R² is independently selected from a naturally or non-naturally occurring nucleobase, which, when taken together, forms a targeting sequence of 16-23 bases in length. In an embodiment of Formula (III) and/or (IV), each R² is independently selected from a naturally or non-naturally occurring nucleobase, which, when taken together, forms a targeting sequence of 16-22 bases in length. In an embodiment of Formula (III) and/or (IV), each R² is independently selected from a naturally or non-naturally occurring nucleobase, which, when taken together, forms a targeting sequence of 16-21 bases in length. In an embodiment of Formula (III) and/or (IV), each R² is independently selected from a naturally or non-naturally occurring nucleobase, which, when taken together, forms a targeting sequence of 16-20 bases in length. In an embodiment of Formula (III) and/or (IV), each R² is independently selected from a naturally or non-naturally occurring nucleobase, which, when taken together, forms a targeting sequence of 16-19 bases in length. In an embodiment of Formula (III) and/or (IV), each R² is independently selected from a naturally or non-naturally occurring nucleobase, which, when taken together, forms a targeting sequence of 16-18 bases in length. In an embodiment of Formula (III) and/or (IV), each R² is independently selected from a naturally or non-naturally occurring nucleobase, which, when taken together, forms a targeting sequence of 16-17 bases in length.

In another embodiment of Formula (III) and/or (IV), each R² is independently selected from a naturally or non-naturally occurring nucleobase, which, when taken together, forms a targeting sequence of 17-30 bases in length. In an embodiment of Formula (III) and/or (IV), each R² is independently selected from a naturally or non-naturally occurring nucleobase, which, when taken together, forms a targeting sequence of 17-29 bases in length. In an embodiment of Formula (III) and/or (IV), each R² is independently selected from a naturally or non-naturally occurring nucleobase, which, when taken together, forms a targeting sequence of 17-28 bases in length. In an embodiment of Formula (III) and/or (IV), each R² is independently selected from a naturally or non-naturally occurring nucleobase, which, when taken together, forms a targeting sequence of 17-27 bases in length. In an embodiment of Formula (III) and/or (IV), each R² is independently selected from a naturally or non-naturally occurring nucleobase, which, when taken together, forms a targeting sequence of 17-26 bases in length. In an embodiment of Formula (III) and/or (IV), each R² is independently selected from a naturally or non-naturally occurring nucleobase, which, when taken together, forms a targeting sequence of 17-25 bases in length. In an embodiment of Formula (III) and/or (IV), each R² is independently selected from a naturally or non-naturally occurring nucleobase, which, when taken together, forms a targeting sequence of 17-24 bases in length. In an embodiment of Formula (III) and/or (IV), each R² is independently selected from a naturally or non-naturally occurring nucleobase, which, when taken together, forms a targeting sequence of 17-23 bases in length. In an embodiment of Formula (III) and/or (IV), each R² is independently selected from a naturally or non-naturally occurring nucleobase, which, when taken together, forms a targeting sequence of 17-22 bases in length. In an embodiment of Formula (III) and/or (IV), each R² is independently selected from a naturally or non-naturally occurring nucleobase, which, when taken together, forms a targeting sequence of 17-21 bases in length. In an embodiment of Formula (III) and/or (IV), each R² is independently selected from a naturally or non-naturally occurring nucleobase, which, when taken together, forms a targeting sequence of 17-20 bases in length. In an embodiment of Formula (III) and/or (IV), each R² is independently selected from a naturally or non-naturally occurring nucleobase, which, when taken together, forms a targeting sequence of 17-19 bases in length. In an embodiment of Formula (III) and/or (IV), each R² is independently selected from a naturally or non-naturally occurring nucleobase, which, when taken together, forms a targeting sequence of 17-18 bases in length.

In another embodiment of Formula (III) and/or (IV), each R² is independently selected from a naturally or non-naturally occurring nucleobase, which, when taken together, forms a targeting sequence of 18-30 bases in length. In an embodiment of Formula (III) and/or (IV), each R² is independently selected from a naturally or non-naturally occurring nucleobase, which, when taken together, forms a targeting sequence of 18-29 bases in length. In an embodiment of Formula (III) and/or (IV), each R² is independently selected from a naturally or non-naturally occurring nucleobase, which, when taken together, forms a targeting sequence of 18-28 bases in length. In an embodiment of Formula (III) and/or (IV), each R² is independently selected from a naturally or non-naturally occurring nucleobase, which, when taken together, forms a targeting sequence of 18-27 bases in length. In an embodiment of Formula (III) and/or (IV), each R² is independently selected from a naturally or non-naturally occurring nucleobase, which, when taken together, forms a targeting sequence of 18-26 bases in length. In an embodiment of Formula (III) and/or (IV), each R² is independently selected from a naturally or non-naturally occurring nucleobase, which, when taken together, forms a targeting sequence of 18-25 bases in length. In an embodiment of Formula (III) and/or (IV), each R² is independently selected from a naturally or non-naturally occurring nucleobase, which, when taken together, forms a targeting sequence of 18-24 bases in length. In an embodiment of Formula (III) and/or (IV), each R² is independently selected from a naturally or non-naturally occurring nucleobase, which, when taken together, forms a targeting sequence of 18-23 bases in length. In an embodiment of Formula (III) and/or (IV), each R² is independently selected from a naturally or non-naturally occurring nucleobase, which, when taken together, forms a targeting sequence of 18-22 bases in length. In an embodiment of Formula (III) and/or (IV), each R² is independently selected from a naturally or non-naturally occurring nucleobase, which, when taken together, forms a targeting sequence of 18-21 bases in length. In an embodiment of Formula (III) and/or (IV), each R² is independently selected from a naturally or non-naturally occurring nucleobase, which, when taken together, forms a targeting sequence of 18-20 bases in length. In an embodiment of Formula (III) and/or (IV), each R² is independently selected from a naturally or non-naturally occurring nucleobase, which, when taken together, forms a targeting sequence of 18-19 bases in length.

In another embodiment of Formula (III) and/or (IV), each R² is independently selected from a naturally or non-naturally occurring nucleobase, which, when taken together, forms a targeting sequence of 19-30 bases in length. In an embodiment of Formula (III) and/or (IV), each R² is independently selected from a naturally or non-naturally occurring nucleobase, which, when taken together, forms a targeting sequence of 19-29 bases in length. In an embodiment of Formula (III) and/or (IV), each R² is independently selected from a naturally or non-naturally occurring nucleobase, which, when taken together, forms a targeting sequence of 19-28 bases in length. In an embodiment of Formula (III) and/or (IV), each R² is independently selected from a naturally or non-naturally occurring nucleobase, which, when taken together, forms a targeting sequence of 19-27 bases in length. In an embodiment of Formula (III) and/or (IV), each R² is independently selected from a naturally or non-naturally occurring nucleobase, which, when taken together, forms a targeting sequence of 19-26 bases in length. In an embodiment of Formula (III) and/or (IV), each R² is independently selected from a naturally or non-naturally occurring nucleobase, which, when taken together, forms a targeting sequence of 19-25 bases in length. In an embodiment of Formula (III) and/or (IV), each R² is independently selected from a naturally or non-naturally occurring nucleobase, which, when taken together, forms a targeting sequence of 19-24 bases in length. In an embodiment of Formula (III) and/or (IV), each R² is independently selected from a naturally or non-naturally occurring nucleobase, which, when taken together, forms a targeting sequence of 19-23 bases in length. In an embodiment of Formula (III) and/or (IV), each R² is independently selected from a naturally or non-naturally occurring nucleobase, which, when taken together, forms a targeting sequence of 19-22 bases in length. In an embodiment of Formula (III) and/or (IV), each R² is independently selected from a naturally or non-naturally occurring nucleobase, which, when taken together, forms a targeting sequence of 19-21 bases in length. In an embodiment of Formula (III) and/or (IV), each R² is independently selected from a naturally or non-naturally occurring nucleobase, which, when taken together, forms a targeting sequence of 19-20 bases in length.

In another embodiment of Formula (III) and/or (IV), each R² is independently selected from a naturally or non-naturally occurring nucleobase, which, when taken together, forms a targeting sequence of 20-30 bases in length. In an embodiment of Formula (III) and/or (IV), each R² is independently selected from a naturally or non-naturally occurring nucleobase, which, when taken together, forms a targeting sequence of 20-29 bases in length. In an embodiment of Formula (III) and/or (IV), each R² is independently selected from a naturally or non-naturally occurring nucleobase, which, when taken together, forms a targeting sequence of 20-28 bases in length. In an embodiment of Formula (III) and/or (IV), each R² is independently selected from a naturally or non-naturally occurring nucleobase, which, when taken together, forms a targeting sequence of 20-27 bases in length. In an embodiment of Formula (III) and/or (IV), each R² is independently selected from a naturally or non-naturally occurring nucleobase, which, when taken together, forms a targeting sequence of 20-26 bases in length. In an embodiment of Formula (III) and/or (IV), each R² is independently selected from a naturally or non-naturally occurring nucleobase, which, when taken together, forms a targeting sequence of 20-25 bases in length. In an embodiment of Formula (III) and/or (IV), each R² is independently selected from a naturally or non-naturally occurring nucleobase, which, when taken together, forms a targeting sequence of 20-24 bases in length. In an embodiment of Formula (III) and/or (IV), each R² is independently selected from a naturally or non-naturally occurring nucleobase, which, when taken together, forms a targeting sequence of 20-23 bases in length. In an embodiment of Formula (III) and/or (IV), each R² is independently selected from a naturally or non-naturally occurring nucleobase, which, when taken together, forms a targeting sequence of 20-22 bases in length. In an embodiment of Formula (III) and/or (IV), each R² is independently selected from a naturally or non-naturally occurring nucleobase, which, when taken together, forms a targeting sequence of 20-21 bases in length.

In another embodiment of Formula (III) and/or (IV), each R² is independently selected from a naturally or non-naturally occurring nucleobase, which, when taken together, forms a targeting sequence of 21-30 bases in length. In an embodiment of Formula (III) and/or (IV), each R² is independently selected from a naturally or non-naturally occurring nucleobase, which, when taken together, forms a targeting sequence of 21-29 bases in length. In an embodiment of Formula (III) and/or (IV), each R² is independently selected from a naturally or non-naturally occurring nucleobase, which, when taken together, forms a targeting sequence of 21-28 bases in length. In an embodiment of Formula (III) and/or (IV), each R² is independently selected from a naturally or non-naturally occurring nucleobase, which, when taken together, forms a targeting sequence of 21-27 bases in length. In an embodiment of Formula (III) and/or (IV), each R² is independently selected from a naturally or non-naturally occurring nucleobase, which, when taken together, forms a targeting sequence of 21-26 bases in length. In an embodiment of Formula (III) and/or (IV), each R² is independently selected from a naturally or non-naturally occurring nucleobase, which, when taken together, forms a targeting sequence of 21-25 bases in length. In an embodiment of Formula (III) and/or (IV), each R² is independently selected from a naturally or non-naturally occurring nucleobase, which, when taken together, forms a targeting sequence of 21-24 bases in length. In an embodiment of Formula (III) and/or (IV), each R² is independently selected from a naturally or non-naturally occurring nucleobase, which, when taken together, forms a targeting sequence of 21-23 bases in length. In an embodiment of Formula (III) and/or (IV), each R² is independently selected from a naturally or non-naturally occurring nucleobase, which, when taken together, forms a targeting sequence of 21-22 bases in length.

In another embodiment of Formula (III) and/or (IV), each R² is independently selected from a naturally or non-naturally occurring nucleobase, which, when taken together, forms a targeting sequence of 22-30 bases in length. In an embodiment of Formula (III) and/or (IV), each R² is independently selected from a naturally or non-naturally occurring nucleobase, which, when taken together, forms a targeting sequence of 22-29 bases in length. In an embodiment of Formula (III) and/or (IV), each R² is independently selected from a naturally or non-naturally occurring nucleobase, which, when taken together, forms a targeting sequence of 22-28 bases in length. In an embodiment of Formula (III) and/or (IV), each R² is independently selected from a naturally or non-naturally occurring nucleobase, which, when taken together, forms a targeting sequence of 22-27 bases in length. In an embodiment of Formula (III) and/or (IV), each R² is independently selected from a naturally or non-naturally occurring nucleobase, which, when taken together, forms a targeting sequence of 22-26 bases in length. In an embodiment of Formula (III) and/or (IV), each R² is independently selected from a naturally or non-naturally occurring nucleobase, which, when taken together, forms a targeting sequence of 22-25 bases in length. In an embodiment of Formula (III) and/or (IV), each R² is independently selected from a naturally or non-naturally occurring nucleobase, which, when taken together, forms a targeting sequence of 22-24 bases in length. In an embodiment of Formula (III) and/or (IV), each R² is independently selected from a naturally or non-naturally occurring nucleobase, which, when taken together, forms a targeting sequence of 22-23 bases in length.

In another embodiment of Formula (III) and/or (IV), each R² is independently selected from a naturally or non-naturally occurring nucleobase, which, when taken together, forms a targeting sequence of 23-30 bases in length. In an embodiment of Formula (III) and/or (IV), each R² is independently selected from a naturally or non-naturally occurring nucleobase, which, when taken together, forms a targeting sequence of 23-29 bases in length. In an embodiment of Formula (III) and/or (IV), each R² is independently selected from a naturally or non-naturally occurring nucleobase, which, when taken together, forms a targeting sequence of 23-28 bases in length. In an embodiment of Formula (III) and/or (IV), each R² is independently selected from a naturally or non-naturally occurring nucleobase, which, when taken together, forms a targeting sequence of 23-27 bases in length. In an embodiment of Formula (III) and/or (IV), each R² is independently selected from a naturally or non-naturally occurring nucleobase, which, when taken together, forms a targeting sequence of 23-26 bases in length. In an embodiment of Formula (III) and/or (IV), each R² is independently selected from a naturally or non-naturally occurring nucleobase, which, when taken together, forms a targeting sequence of 23-25 bases in length. In an embodiment of Formula (III) and/or (IV), each R² is independently selected from a naturally or non-naturally occurring nucleobase, which, when taken together, forms a targeting sequence of 23-24 bases in length.

In another embodiment of Formula (III) and/or (IV), each R² is independently selected from a naturally or non-naturally occurring nucleobase, which, when taken together, forms a targeting sequence of 24-30 bases in length. In an embodiment of Formula (III) and/or (IV), each R² is independently selected from a naturally or non-naturally occurring nucleobase, which, when taken together, forms a targeting sequence of 24-29 bases in length. In an embodiment of Formula (III) and/or (IV), each R² is independently selected from a naturally or non-naturally occurring nucleobase, which, when taken together, forms a targeting sequence of 24-28 bases in length. In an embodiment of Formula (III) and/or (IV), each R² is independently selected from a naturally or non-naturally occurring nucleobase, which, when taken together, forms a targeting sequence of 24-27 bases in length. In an embodiment of Formula (III) and/or (IV), each R² is independently selected from a naturally or non-naturally occurring nucleobase, which, when taken together, forms a targeting sequence of 24-26 bases in length. In an embodiment of Formula (III) and/or (IV), each R² is independently selected from a naturally or non-naturally occurring nucleobase, which, when taken together, forms a targeting sequence of 24-25 bases in length.

In another embodiment of Formula (III) and/or (IV), each R² is independently selected from a naturally or non-naturally occurring nucleobase, which, when taken together, forms a targeting sequence of 25-30 bases in length. In an embodiment of Formula (III) and/or (IV), each R² is independently selected from a naturally or non-naturally occurring nucleobase, which, when taken together, forms a targeting sequence of 25-29 bases in length. In an embodiment of Formula (III) and/or (IV), each R² is independently selected from a naturally or non-naturally occurring nucleobase, which, when taken together, forms a targeting sequence of 25-28 bases in length. In an embodiment of Formula (III) and/or (IV), each R² is independently selected from a naturally or non-naturally occurring nucleobase, which, when taken together, forms a targeting sequence of 25-27 bases in length. In an embodiment of Formula (III) and/or (IV), each R² is independently selected from a naturally or non-naturally occurring nucleobase, which, when taken together, forms a targeting sequence of 25-26 bases in length.

In another embodiment of Formula (III) and/or (IV), each R² is independently selected from a naturally or non-naturally occurring nucleobase, which, when taken together, forms a targeting sequence of 26-30 bases in length. In an embodiment of Formula (III) and/or (IV), each R² is independently selected from a naturally or non-naturally occurring nucleobase, which, when taken together, forms a targeting sequence of 26-29 bases in length. In an embodiment of Formula (III) and/or (IV), each R² is independently selected from a naturally or non-naturally occurring nucleobase, which, when taken together, forms a targeting sequence of 26-28 bases in length. In an embodiment of Formula (III) and/or (IV), each R² is independently selected from a naturally or non-naturally occurring nucleobase, which, when taken together, forms a targeting sequence of 26-27 bases in length.

In another embodiment of Formula (III) and/or (IV), each R² is independently selected from a naturally or non-naturally occurring nucleobase, which, when taken together, forms a targeting sequence of 27-30 bases in length. In an embodiment of Formula (III) and/or (IV), each R² is independently selected from a naturally or non-naturally occurring nucleobase, which, when taken together, forms a targeting sequence of 27-29 bases in length. In an embodiment of Formula (III) and/or (IV), each R² is independently selected from a naturally or non-naturally occurring nucleobase, which, when taken together, forms a targeting sequence of 27-28 bases in length.

In another embodiment of Formula (III) and/or (IV), each R² is independently selected from a naturally or non-naturally occurring nucleobase, which, when taken together, forms a targeting sequence of 28-30 bases in length. In an embodiment of Formula (III) and/or (IV), each R² is independently selected from a naturally or non-naturally occurring nucleobase, which, when taken together, forms a targeting sequence of 28-29 bases in length. In another embodiment of Formula (III) and/or (IV), each R² is independently selected from a naturally or non-naturally occurring nucleobase, which, when taken together, forms a targeting sequence of 28-30 bases in length. In an embodiment of Formula (III) and/or (IV), each R² is independently selected from a naturally or non-naturally occurring nucleobase, which, when taken together, forms a targeting sequence of 29-30 bases in length.

In another embodiment of Formula (III) and/or (IV), each R² is independently selected from a naturally or non-naturally occurring nucleobase, which, when taken together, forms a targeting sequence of 13 bases in length.

In another embodiment of Formula (III) and/or (IV), each R² is independently selected from a naturally or non-naturally occurring nucleobase, which, when taken together, forms a targeting sequence of 14 bases in length. In another embodiment of Formula (III) and/or (IV), each R² is independently selected from a naturally or non-naturally occurring nucleobase, which, when taken together, forms a targeting sequence of 15 bases in length. In another embodiment of Formula (III) and/or (IV), each R² is independently selected from a naturally or non-naturally occurring nucleobase, which, when taken together, forms a targeting sequence of 16 bases in length. In another embodiment of Formula (III) and/or (IV), each R² is independently selected from a naturally or non-naturally occurring nucleobase, which, when taken together, forms a targeting sequence of 17 bases in length. In another embodiment of Formula (III) and/or (IV), each R² is independently selected from a naturally or non-naturally occurring nucleobase, which, when taken together, forms a targeting sequence of 18 bases in length. In another embodiment of Formula (III) and/or (IV), each R² is independently selected from a naturally or non-naturally occurring nucleobase, which, when taken together, forms a targeting sequence of 19 bases in length. In another embodiment of Formula (III) and/or (IV), each R² is independently selected from a naturally or non-naturally occurring nucleobase, which, when taken together, forms a targeting sequence of 20 bases in length. In another embodiment of Formula (III) and/or (IV), each R² is independently selected from a naturally or non-naturally occurring nucleobase, which, when taken together, forms a targeting sequence of 21 bases in length. In another embodiment of Formula (III) and/or (IV), each R² is independently selected from a naturally or non-naturally occurring nucleobase, which, when taken together, forms a targeting sequence of 22 bases in length. In another embodiment of Formula (III) and/or (IV), each R² is independently selected from a naturally or non-naturally occurring nucleobase, which, when taken together, forms a targeting sequence of 23 bases in length. In another embodiment of Formula (III) and/or (IV), each R² is independently selected from a naturally or non-naturally occurring nucleobase, which, when taken together, forms a targeting sequence of 24 bases in length. In another embodiment of Formula (III) and/or (IV), each R² is independently selected from a naturally or non-naturally occurring nucleobase, which, when taken together, forms a targeting sequence of 25 bases in length. In another embodiment of Formula (III) and/or (IV), each R² is independently selected from a naturally or non-naturally occurring nucleobase, which, when taken together, forms a targeting sequence of 26 bases in length. In another embodiment of Formula (III) and/or (IV), each R² is independently selected from a naturally or non-naturally occurring nucleobase, which, when taken together, forms a targeting sequence of 27 bases in length. In another embodiment of Formula (III) and/or (IV), each R² is independently selected from a naturally or non-naturally occurring nucleobase, which, when taken together, forms a targeting sequence of 28 bases in length. In another embodiment of Formula (III) and/or (IV), each R² is independently selected from a naturally or non-naturally occurring nucleobase, which, when taken together, forms a targeting sequence of 29 bases in length. In another embodiment of Formula (III) and/or (IV), each R² is independently selected from a naturally or non-naturally occurring nucleobase, which, when taken together, forms a targeting sequence of 30 bases in length.

In an embodiment, the antisense oligomer of Formula (III) and/or Formula (IV) comprises a targeting sequence complementary to a target region within a pre-mRNA of the human UMOD gene. In another embodiment, the antisense oligomer, or a pharmaceutically acceptable salt thereof, comprises a targeting sequence complementary to a target region within a pre-mRNA of the human UMOD gene. In certain embodiments, the target region is an intron/exon junction or an exon internal region of exon 2 (SEQ ID NO: 2), exon 5 (SEQ ID NO: 3), exon 6 (SEQ ID NO: 4), exon 8 (SEQ ID NO: 5) or exon 9 (SEQ ID NO: 6).

In some embodiments, the target region is an intron/exon junction of the human UMOD gene pre-mRNA. In other embodiments, the target region is an exon internal region of the human UMOD gene pre-mRNA.

In an embodiment, the target region is an intron/exon junction or an exon internal region of exon 2. In certain embodiments, the target region is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175). In certain embodiments, the antisense oligomer of Formula (III) and/or Formula (IV), has a targeting sequence comprising a

| SEQ ID NO: 11 | |
|---|---|
| | (GTCTGGTGTCCTGTGAACAGAGATG); |
| SEQ ID NO: 12 | |
| | (CTCTCTGTCTCTGATGTCTGGTGTC); |
| SEQ ID NO: 13 | |
| | (AGACGGGTTGGCCCTTTGAATTTTT); |
| SEQ ID NO: 14 | |
| | (TCTAGATAGCACCTGCCCAAAGGAA); |
| SEQ ID NO: 15 | |
| | (ATCCTTTCTGCTCTTCCCGC); |
| SEQ ID NO: 16 | |
| | (CATCCTTTCTGCTCTTCCCG); |
| SEQ ID NO: 17 | |
| | (AGAGATGGCTGCCCCATCCTTTCTG); |
| SEQ ID NO: 20 | |
| | (CAAGTCAGAGATGGCTGCCCCATCC); |
| SEQ ID NO: 23 | |
| | (CATCCAAGTCAGAGATGGCT); |
| SEQ ID NO: 26 | |
| | (ACCATCAGCATCCAAGTCAG); |
| SEQ ID NO: 27 | |
| | (AGAGGCCACCACCACCATCAGCATC); |
| | and |
| SEQ ID NO: 28 | |
| | (CAGTGGCTGCAGTTGTGATGAACCA). |

In an embodiment, the antisense oligomers disclosed herein, or pharmaceutically acceptable salts thereof, including antisense oligomers, or pharmaceutically acceptable salts thereof, of Formula (III) and/or Formula (IV), have a targeting sequence complementary to a target region within the 51^{st} nucleotide to the 119^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 154. In other embodiments, the antisense oligomers disclosed herein, or pharmaceutically acceptable salts thereof, including antisense oligomers, or pharmaceutically acceptable salts thereof, of Formula (III) and/or Formula (IV), have a targeting sequence complementary to a target region within the 51^{st} nucleotide to the 105^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 155. In still a further embodiment, the target region is H2A(+51+75). In certain embodiments, the antisense oligomer, or a pharmaceutically acceptable salt thereof, has a targeting sequence comprising or consisting of SEQ ID NO: 14.

In an embodiment, the antisense oligomers disclosed herein, or pharmaceutically acceptable salts thereof, including antisense oligomers, or pharmaceutically acceptable salts thereof, of Formula (III) and/or Formula (IV), have a targeting sequence complementary to a target region within the 85^{th} nucleotide to the 119^{th} nucleotide, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 156. In a further embodiment, the target region is selected from H2A(+85+104), H2A(+86+105), and H2A(+95+119). In still a further embodiment, the antisense oligomer, or a pharmaceutically acceptable salt thereof, has a targeting sequence comprising or consisting of a sequence selected from SEQ ID NOs: 15-17.

In an embodiment, the antisense oligomers disclosed herein, or pharmaceutically acceptable salts thereof, including antisense oligomers, or pharmaceutically acceptable salts thereof, of Formula (III) and/or Formula (IV), have a targeting sequence complementary to a target region within the 15^{th} nucleotide of intron 1, as measured from the 5′ end of exon 2, and the 25^{th} nucleotide of exon 2, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 157. In a further embodiment, the target region is selected from H2A(−15+10) and H2A(+1+25). In some embodiments, the antisense oligomer, or a pharmaceutically acceptable salt thereof, has a targeting sequence comprising or consisting of SEQ ID NO: 12 or 13.

In an embodiment, the antisense oligomers disclosed herein, or pharmaceutically acceptable salts thereof, including antisense oligomers, or pharmaceutically acceptable salts thereof, of Formula (III) and/or Formula (IV), have a targeting sequence complementary to a target region within the 118^{th} nucleotide to the 150^{th} nucleotide of exon 2, as measured from the 5′ end of exon 2, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 158. In a further embodiment, the target region is selected from H2A(+118+137) and H2A(+126+150). In certain embodiments, the antisense oligomers, or pharmaceutically acceptable salts thereof, have a targeting sequence comprising or consisting of SEQ ID NO: 26 or 27.

In an embodiment, the antisense oligomers disclosed herein, or pharmaceutically acceptable salts thereof, including antisense oligomers, or pharmaceutically acceptable salts thereof, of Formula (III) and/or Formula (IV), have a targeting sequence complementary to a target region of exon 2 selected from H2A(+101+125), H2A(+110+129), and H2A(+151+175). In a further embodiment, the antisense oligomer, or a pharmaceutically acceptable salt thereof, has a targeting sequence comprising or consisting of a sequence selected from SEQ ID NOs: 20, 21, and 28.

In another embodiment, the target region is an intron/exon junction or an exon internal region of exon 5. In certain embodiments, the target region is selected from H5A(−15+5), H5A(−14+6), H5A(−11+9), H5A(−10+10), H5A(+51+75), H5A(+76+100), H5A(+78+95), H5A(+80+97), H5A(+82+99), H5A(+126+150), H5A(+153+172), H5A(+154+173), H5D(+18-2), H5D(+16-4), H5D(+14-6), H5D(+13-7), and H5D(+12-8). In certain embodiments, the antisense oligomer of Formula (III) and/or Formula (IV), or a pharmaceutically acceptable salt thereof, has a targeting sequence comprising a sequence selected from:

| SEQ ID NO: 30 | |
|---|---|
| | (GATATCTGAAACAGGTTAGG); |
| SEQ ID NO: 31 | |
| | (AGATATCTGAAACAGGTTAG); |
| SEQ ID NO: 32 | |
| | (GGGAGATATCTGAAACAGGT); |
| SEQ ID NO: 33 | |
| | (AGGGAGATATCTGAAACAGG); |
| SEQ ID NO: 35 | |
| | (TCAGCTGGCACTTGCCCAGCGACAC); |
| SEQ ID NO: 37 | |
| | (AAGACCTTGTCGAAGCCCAGACTCT); |
| SEQ ID NO: 38 | |
| | (CTTGTCGAAGCCCAGACT); |
| SEQ ID NO: 39 | |
| | (ACCTTGTCGAAGCCCAGA); |
| SEQ ID NO: 40 | |
| | (AGACCTTGTCGAAGCCCA); |
| SEQ ID NO: 44 | |
| | (GGTTGTCTCTGTCATTGAAGCCCGA); |
| SEQ ID NO: 46 | |
| | (GTCACTACAGACACCCAGTC); |
| SEQ ID NO: 47 | |
| | (GGTCACTACAGACACCCAGT); |
| SEQ ID NO: 48 | |
| | (ACCGTCAACACTGTCCCACA); |
| SEQ ID NO: 50 | |
| | (GTACCGTCAACACTGTCCCA); |
| SEQ ID NO: 51 | |
| | (ACGTACCGTCAACACTGTCC); |
| SEQ ID NO: 52 | |
| | (GACGTACCGTCAACACTGTC); |
| | and |
| SEQ ID NO: 53 | |
| | (GGACGTACCGTCAACACTGT). |

In an embodiment, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region within the 15^{th} nucleotide of intron 4, as measured from the 5′ end of exon 5 and the 10^{th} nucleotide of exon 5, as measured from the 5′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 159. In a further embodiment, the target region is selected from H5A(−15+5), H5A(−14+6), H5A(−11+9), and H5A(−10+10). In still a further embodiment, the antisense oligomers, or pharmaceutically acceptable salts thereof, disclosed herein, including antisense oligomers, or pharmaceutically acceptable salts thereof, of Formula (III) and/or Formula (IV), have a targeting sequence comprising or consisting of a sequence selected from SEQ ID NOs: 30-33.

In an embodiment, the target region is H5A(−15+5). In another embodiment, the targeting sequence comprises SEQ ID NO: 30.

In an embodiment, the target region is H5A(−14+6). In another embodiment, the target region is H5A(−15+5). In certain embodiments the targeting sequence comprises SEQ ID NO: 31.

In an embodiment, the target region is H5A(−11+9). In another embodiment, the target region is H5A(−11+9). In some embodiments, the targeting sequence comprises SEQ ID NO: 32.

In an embodiment, the target region is H5A(−10+10). In another embodiment, the targeting sequence comprises SEQ ID NO: 33.

In an embodiment, antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to the target region H5A(+51+75). In a further embodiment, the antisense oligomers, or pharmaceutically acceptable salts thereof, disclosed herein, including antisense oligomers, or pharmaceutically acceptable salts thereof, of Formula (III) and/or Formula (IV), the targeting sequence comprises SEQ ID NO: 35.

In an embodiment, the antisense oligomers, or pharmaceutically acceptable salts thereof, of the disclosed herein, including antisense oligomers, or pharmaceutically acceptable salts thereof, of Formula (III) and/or Formula (IV), have a targeting sequence complementary to a target region within the 76^{th} nucleotide to the 100^{th} nucleotide, as measured from the 5′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 37. In a further embodiment, the target region is selected from H5A(+76+100), H5A(+78+95), H5A(+80+97), and H5A(+82+99). In one embodiment, the target region is H5A(+76+100). In another embodiment, the targeting sequence comprises SEQ ID NO: 37.

In one embodiment, the target region is H5A(+78+95). In another embodiment, the antisense oligomer, or a pharmaceutically acceptable salt thereof, has a targeting sequence comprising SEQ ID NO: 38.

In one embodiment, the target region is H5A(+80+97). In another embodiment, the antisense oligomer, or a pharmaceutically acceptable salt thereof, has a targeting sequence comprising SEQ ID NO: 39.

In one embodiment, the target region is H5A(+82+99). In another embodiment, the antisense oligomer, or a pharmaceutically acceptable salt thereof, has a targeting sequence comprising SEQ ID NO: 40.

In an embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 18^{th} nucleotide of exon 5 measured from 3′ end of exon 5 to the 8^{th} nucleotide of intron 5 measured from 3′ end of exon 5, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 160. In a further embodiment, the target region is selected from H5D(+18-2), H5D(+16-4), H5D(+14-6), H5D(+13-7), and H5D(+12-8). In still a further embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence comprising a sequence selected from SEQ ID NOs: 48 and 50-53.

In another embodiment, the target region is an intron/exon junction or an exon internal region of exon 6. In certain embodiments, the target region is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D(+24-1), H6D(+15-5), and H6D(+14-6). In certain embodiments, the antisense oligomer of Formula (III) and/or Formula (IV), or a pharmaceutically acceptable salt thereof, has a targeting sequence comprising a sequence selected from:

| SEQ ID NO: 57 | |
|---|---|
| | (TCATCTGCCAGGTAGAGGGTGTTGC); |
| SEQ ID NO: 58 | |
| | (GGTCACGGATGATGATCTCA); |
| SEQ ID NO: 59 | |
| | (AGGTCACGGATGATGATCTC); |
| SEQ ID NO: 61 | |
| | (TTGATGTTGAGGTCACGGAT); |
| SEQ ID NO: 62 | |
| | (TTTGATGTTGAGGTCACGGA); |
| SEQ ID NO: 64 | |
| | (GGTAGGAGCATGCAAAGTTGATTTT); |
| SEQ ID NO: 66 | |
| | (TTCAGGCTGACTTTCATGTCCAGGG); |
| SEQ ID NO: 67 | |
| | (GCTGACTTTCATGTCCAGGG); |
| SEQ ID NO: 68 | |
| | (GGTCTTCAGGCTGACTTTCA); |
| SEQ ID NO: 69 | |
| | (CGGTCTTCAGGCTGACTTTC); |
| SEQ ID NO: 70 | |
| | (GCGGTCTTCAGGCTGACTTT); |
| SEQ ID NO: 71 | |
| | (GGCGGTCTTCAGGCTGACTT); |
| SEQ ID NO: 72 | |
| | (CTGTAGGGCGGTCTTCAGGC); |
| SEQ ID NO: 73 | |
| | (GCTGTAGGGCGGTCTTCAGG); |
| SEQ ID NO: 74 | |
| | (GGCTGTAGGGCGGTCTTCAG); |
| SEQ ID NO: 75 | |
| | (TGGCTGTAGGGCGGTCTTCA); |
| SEQ ID NO: 76 | |
| | (TTGGCTGTAGGGCGGTCTTC); |
| SEQ ID NO: 77 | |
| | (ATTGGCTGTAGGGCGGTCTT); |
| SEQ ID NO: 78 | |
| | (CTGACCATTGGCTGTAGGGC); |
| SEQ ID NO: 79 | |
| | (CCTGACCATTGGCTGTAGGGCGGTC); |
| SEQ ID NO: 80 | |
| | (CACACCTGACCATTGGCTGT); |
| | and |
| SEQ ID NO: 81 | |
| | (CCACACCTGACCATTGGCTG). |

In an embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to the target region H6A(+26+50). In a further embodiment, antisense oligomers, or pharmaceutically acceptable salts thereof, disclosed herein, including antisense oligomers, or pharmaceutically acceptable salts thereof, of Formula (III) and/or Formula (IV), the targeting sequence comprises SEQ ID NO: 57.

In an embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 48^{th} nucleotide to the 78^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 161. In a further embodiment, the target region is selected from H6A(+48+67), H6A(+49+68), H6A(+58+77), and H6A(+59+78). In still a further embodiment, the antisense oligomer, or a pharmaceutically acceptable salt thereof, has a targeting sequence comprising a sequence selected from SEQ ID NOs: 58, 59, 61, and 62.

In another embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 58^{th} nucleotide to the 78^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 162. In certain embodiments, the target region is selected from H6A(+58+77) and H6A(+59+78). In one embodiment, the target region is H6A(+58+77). In one embodiment, the antisense oligomer, or a pharmaceutically acceptable salt thereof, has a targeting sequence comprising SEQ ID NO: 61. In another embodiment, the target region is H6A(+59+78). In one embodiment, the antisense oligomer, or a pharmaceutically acceptable salt thereof, has a targeting sequence comprising SEQ ID NO: 62.

In an embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to the target region H6A(+76+100). In a further embodiment, the antisense oligomer, or a pharmaceutically acceptable salt thereof, has a targeting sequence complementary to the target region H6A(+26+50). In another embodiment, the targeting sequence comprises SEQ ID NO: 64.

In an embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 101^{st} nucleotide to the 132^{nd} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 163. In a further embodiment, the target region is selected from H6A(+101+125), H6A(+101+120), H6A(+110+129), H6A(+111+130), H6A(+112+131), and H6A(+113+132). In still a further embodiment, the antisense oligomers, or pharmaceutically acceptable salts thereof, disclosed herein, including antisense oligomers, or pharmaceutically acceptable salts thereof, of Formula (III) and/or Formula (IV), have a targeting sequence comprising a sequence selected from SEQ ID NOs: 66-71.

In another embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 111^{th} nucleotide to the 132^{nd} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 164. In certain embodiments, the target region is selected from H6A(+111+130), H6A(+112+131), and H6A(+113+132). In one embodiment, the target region is H6A(+111+130). In one embodiment, the antisense oligomer, or a pharmaceutically acceptable salt thereof, has a targeting sequence comprising SEQ ID NO: 69. In another embodiment, the target region is H6A(+112+131). In certain embodiments, the antisense oligomer, or a pharmaceutically acceptable salt thereof, has a targeting sequence comprising SEQ ID NO: 70. In yet another embodiment, the target region is H6A(+113+133). In certain embodiments, the antisense oligomer, or a pharmaceutically acceptable salt thereof, has a targeting sequence comprising SEQ ID NO: 71.

In an embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 119^{th} nucleotide to the 149^{th} nucleotide measured from the 5′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 165. In another embodiment, the target region is selected from H6A(+119+138), H6A(+120+139), H6A(+121+140), H6A(+122+141), H6A(+123+142), H6A(+124+143), and H6A(+130+149). In yet another embodiment, the antisense oligomer, or a pharmaceutically acceptable salt thereof, has a targeting sequence comprising a sequence selected from SEQ ID NOs: 72-78.

In a further embodiment, the antisense oligomer, or a pharmaceutically acceptable salts thereof, disclosed herein, including the antisense oligomers, or pharmaceutically acceptable salts thereof, of Formula (III) and/or Formula (IV), have a targeting sequence complementary to a target region within the 119^{th} nucleotide to the 141^{st} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 166. In an embodiment, the target region is selected from H6A(+119+138), H6A(+120+139), H6A(+121+140), and

H6A(+122+141). In another embodiment, the antisense oligomer, or a pharmaceutically acceptable salt thereof, has a targeting sequence comprising a sequence selected from SEQ ID NOs: 72-75.

In still a further embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 120^{th} nucleotide to the 141^{st} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 167. In some embodiments, the target region is H6A(+120+139), H6A(+121+140), or H6A(+122+141). In one embodiment, the target region is H6A(+120+139). In another embodiment, the antisense oligomer, or a pharmaceutically acceptable salt thereof, has a targeting sequence comprising SEQ ID NO: 73. In another embodiment, the target region is H6A(+121+140). In certain embodiments, the antisense oligomer, or a pharmaceutically acceptable salt thereof, has a targeting sequence comprising SEQ ID NO: 74. In yet another embodiment, the target region is H6A(+122+141). In certain embodiments, the antisense oligomer, or a pharmaceutically acceptable salt thereof, has a targeting sequence comprising SEQ ID NO: 75.

In still a further embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 123^{rd} nucleotide to the 149^{th} nucleotide measured from the 5′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 168. In an embodiment, the target region is selected from H6A(+123+142), H6A(+124+143), and H6A(+130+149). In another embodiment, the antisense oligomer, or a pharmaceutically acceptable salt thereof, has a targeting sequence comprising a sequence selected from SEQ ID NOs: 76-78.

In an embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 24^{th} nucleotide of exon 6 measured from 3′ end of exon 6 to the 6^{th} nucleotide of intron 6 measured from 3′ end of exon 6 of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 169. In a further embodiment, the target region is selected from H6D(+24-1), H6D(+15-5), and H6D(+14-6). In still a further embodiment, the antisense oligomer, or a pharmaceutically acceptable salt thereof, has a targeting sequence comprising a sequence selected from SEQ ID NOs: 79-81.

In a further embodiment, the antisense oligomers, or pharmaceutically acceptable salts thereof, disclosed herein, including the antisense oligomers, or pharmaceutically acceptable salts thereof, of Formula (III) and/or Formula (IV), have a targeting sequence complementary to a target region within the 15^{th} nucleotide of exon 6 measured from 3′ end of exon 6 to the 6^{th} nucleotide of intron 6 measured from 3′ end of exon 6, of the human UMOD gene pre-mRNA. In certain embodiments, the antisense oligomers, or pharmaceutically acceptable salts thereof, disclosed herein, including antisense oligomers, or pharmaceutically acceptable salts thereof, of Formula (III) and/or Formula (IV), have a targeting sequence complementary to a target region comprised in SEQ ID NO: 170. In some embodiments, the target region is H6D(+15-5), or H6D(+14-6). In one embodiment, the target region is H6D(+15-5). In certain embodiments, the antisense oligomer, or a pharmaceutically acceptable salt thereof, has a targeting sequence comprising SEQ ID NO: 80. In another embodiment, the target region is H6D(+14-6). In certain embodiments, the antisense oligomer, or a pharmaceutically acceptable salt thereof, has a targeting sequence comprising SEQ ID NO: 81.

In yet another embodiment, the target region is an intron/exon junction or an exon internal region of exon 8. In certain embodiments, the target region is selected from H8A(−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D(+12-13).

In certain embodiments, the antisense oligomer of Formula (III) and/or Formula (IV), or a pharmaceutically acceptable salt thereof, has a targeting sequence comprising a sequence selected from:

| SEQ ID NO: 82 | |
|---|---|
| | (TTGAGTCTCTAGTGTGTGGGCATCT); |
| SEQ ID NO: 84 | |
| | (TGGACGGAAAATCGGCCCTGGGAGG); |
| SEQ ID NO: 85 | |
| | (CATCTGGACGGAAAATCGGC); |
| SEQ ID NO: 86 | |
| | (ACATCTGGACGGAAAATCGG); |
| SEQ ID NO: 89 | |
| | (AACCGGAACATCTGGACGGA); |
| SEQ ID NO: 90 | |
| | (AAACCGGAACATCTGGACGG); |
| SEQ ID NO: 91 | |
| | (CAAACCGGAACATCTGGACG); |
| SEQ ID NO: 92 | |
| | (TTCCAGCAAACCGGAACATC); |
| SEQ ID NO: 93 | |
| | (ATAGTTTCCAGCAAACCGGAACATC); |
| SEQ ID NO: 94 | |
| | (TTTCCAGCAAACCGGAACAT); |
| SEQ ID NO: 95 | |
| | (GTTTCCAGCAAACCGGAACA); |
| SEQ ID NO: 96 | |
| | (AGTTTCCAGCAAACCGGAAC); |
| SEQ ID NO: 98 | |
| | (ATAGTTTCCAGCAAACCGGA); |
| SEQ ID NO: 99 | |
| | (CATAGTTTCCAGCAAACCGG); |
| SEQ ID NO: 100 | |
| | (TCATAGTTTCCAGCAAACCG); |
| SEQ ID NO: 101 | |
| | (AGGTCATAGTTTCCAGCAAA); |
| SEQ ID NO: 102 | |
| | (GGTAGACTAGGTCATAGTTT); |
| SEQ ID NO: 103 | |
| | (AGGTAGACTAGGTCATAGTT); |
| SEQ ID NO: 104 | |
| | (CAGGTAGACTAGGTCATAGT); |
| SEQ ID NO: 105 | |
| | (CTTCACAGTGCAGGTAGACTAGGTC); |
| SEQ ID NO: 106 | |
| | (ACAGTGCAGGTAGACTAGGT); |
| SEQ ID NO: 107 | |
| | (CACAGTGCAGGTAGACTAGG); |
| SEQ ID NO: 108 | |
| | (TCACAGTGCAGGTAGACTAG); |
| SEQ ID NO: 109 | |
| | (TTCACAGTGCAGGTAGACTA); |
| SEQ ID NO: 110 | |
| | (GTCACAGAGATAGACTTCAC); |
| SEQ ID NO: 111 | |
| | (TGTCACAGAGATAGACTTCA); |
| SEQ ID NO: 112 | |
| | (GTGTCACAGAGATAGACTTC); |
| SEQ ID NO: 113 | |
| | (TCATTCATGGTGTCACAGAGATAGA); |
| SEQ ID NO: 114 | |
| | (TTCATGGTGTCACAGAGATA); |
| SEQ ID NO: 115 | |
| | (ATTCATGGTGTCACAGAGAT); |
| | and |
| SEQ ID NO: 120 | |
| | (GAGTCAACTCACAGGCTTGCACTTT). |

In an embodiment, the antisense oligomer, or a pharmaceutically acceptable salt thereof, has a targeting sequence complementary to the target region H8A(−2+23). In a further embodiment, the antisense oligomer, or a pharmaceutically acceptable salt thereof, has a targeting sequence comprising SEQ ID NO: 82. In still a further embodiment, the antisense oligomer, or a pharmaceutically acceptable salt thereof, has a targeting sequence complementary to the target region H8A(−2+23), wherein the targeting sequence comprises SEQ ID NO: 82.

In some embodiments, the antisense oligomer, or a pharmaceutically acceptable salt thereof, has a targeting sequence complementary to a target region within the 51^{st} nucleotide to the 80^{th} nucleotide measured from the 5′ end of exon 8 of the human UMOD gene pre-mRNA. In certain embodiments, the antisense oligomer, or pharmaceutically acceptable salts thereof, has a targeting sequence complementary to a target region comprised in SEQ ID NO: 171. In a further embodiment, the target region is selected from H8A(+51+75), H8A(+60+79), and H8A(+61+80). In one embodiment, the antisense oligomer, or a pharmaceutically acceptable salt thereof, has a targeting sequence comprising a sequence selected from SEQ ID NOs: 84-86.

In an embodiment, the antisense oligomer, or a pharmaceutically acceptable salt thereof, has a targeting sequence complementary to a target region within the 68^{th} nucleotide to the 100^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 172. In another embodiment, the target region is selected from H8A(+68+87), H8A(+69+88), H8A(+70+89), H8A(+76+95), H8A(+76+100), H8A(+77+96), H8A(+78+97), and H8A(+79+98). In yet another embodiment, the antisense oligomer, or a pharmaceutically acceptable salt thereof, has a targeting sequence comprising a sequence selected from SEQ ID NOs: 89-96.

In a further embodiment, the antisense oligomer, or a pharmaceutically acceptable salt thereof, has a targeting sequence complementary to a target region within the 76^{th} nucleotide to the 100^{th} nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 173. In an embodiment, the target region is selected from H8A(+76+95), H8A(+77+96), H8A(+78+97), and H8A(+79+98). In another embodiment, the antisense oligomer, or a pharmaceutically acceptable salt thereof, has a targeting sequence comprising a sequence selected from SEQ ID NOs: 92 and 94-96.

In an embodiment, the antisense oligomer, or a pharmaceutically acceptable salt thereof, has a targeting sequence complementary to a target region within the 81^{st} nucleotide to the 105^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 174. In a further embodiment, the target region is selected from H8A(+81+100), H8A(+82+101), H8A(+83+102), and H8A(+86+105). In still a further embodiment, the antisense oligomer, or a pharmaceutically acceptable salt thereof, has a targeting sequence comprising a sequence selected from SEQ ID NOs: 98-101.

In an embodiment, the antisense oligomer, or a pharmaceutically acceptable salt thereof, has a targeting sequence complementary to a target region within the 94^{th} nucleotide to the 124^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 175. In a further embodiment, the target region is selected from H8A(+94+113), H8A(+95+114), H8A(+96+115), H8A(+101+125), H8A(+102+121), H8A(+103+122), H8A(+104+123), and H8A(+105+124). In still a further embodiment, the antisense oligomer, or a pharmaceutically acceptable salt thereof, has a targeting sequence comprising a sequence selected from SEQ ID NOs: 102-109.

In an embodiment, the antisense oligomer, or a pharmaceutically acceptable salt thereof, has a targeting sequence complementary to a target region within the 120^{th} nucleotide to the 148^{th} nucleotide measured from the 5′ end of exon 8 of the human UMOD gene pre-mRNA. In certain embodiments, the antisense oligomer, or a pharmaceutically acceptable salt thereof, has a targeting sequence complementary to a target region comprised in SEQ ID NO: 176. In a further embodiment, the target region is selected from H8A(+120+139), H8A(+121+140), H8A(+122+141), H8A(+126+150), H8A(+128+147), and H8A(+129+148). In still a further embodiment, the antisense oligomer, or a pharmaceutically acceptable salt thereof, has a targeting sequence comprising a sequence selected from SEQ ID NOs: 110-115.

In a further embodiment, the antisense oligomer, or a pharmaceutically acceptable salt thereof, has a targeting sequence complementary to a target region within the 126^{th} nucleotide to the 148^{th} nucleotide measured from the 5′ end of exon 8, of the human UMOD gene pre-mRNA. In certain embodiments, the antisense oligomer, or a pharmaceutically acceptable salt thereof, has a targeting sequence complementary to a target region comprised in SEQ ID NO: 177. In certain embodiments, the target region is selected from H8A(+126+150), H8A(+128+147), and H8A(+129+148). In a further embodiment, the target region is selected from H8A(+126+150), H8A(+128+147), and H8A(+129+148). In some embodiments, the antisense oligomer, or a pharmaceutically acceptable salt thereof, has a targeting sequence comprising a sequence selected from SEQ ID NOs: 113-115. In one embodiment, the target region is H8A(+128+147). In some embodiments, the antisense oligomer, or a pharmaceutically acceptable salt thereof, has a targeting sequence comprising SEQ ID NO: 114.

In an embodiment, the antisense oligomer, or a pharmaceutically acceptable salt thereof, has a targeting sequence complementary to the target region H8D(+12-13). In another embodiment, the target region is H8D(+12-13). In some embodiments, the antisense oligomer, or a pharmaceutically acceptable salt thereof, has a targeting sequence comprising SEQ ID NO: 120.

In still another embodiment, the target region is an intron/exon junction or an exon internal region of exon 9. In certain embodiments, the target region is selected from H9A(−5+20), H9A(+1+25), H9A(+51+75), and H9D(+7-18). In certain embodiments, the antisense oligomer of Formula (III) and/or Formula (IV), or a pharmaceutically acceptable salt thereof, has a targeting sequence comprising a sequence selected from:

| SEQ ID NO: 121 | |
|---|---|
| | (AATCTGGTCCCAGAGCAGGTCTACA); |
| SEQ ID NO: 122 | |
| | (TTCGGAATCTGGTCCCAGAGCAGGT); |
| SEQ ID NO: 123 | |
| | (TGTGATGGGACCCAAGTTCAGGACA); |
| | and |
| SEQ ID NO: 124 | |
| | (AGCGAGTGGCTCTCTTACCTTTCCG). |

In an embodiment, the antisense oligomer, or a pharmaceutically acceptable salt thereof, has a targeting sequence complementary to a target region within the 5^{th} nucleotide of intron 8 measured from the 5′ end of exon 9 and the 25^{th} nucleotide of exon 9 measured from the 5′ end of exon 9 of the human UMOD gene pre-mRNA. In certain embodiments, the antisense oligomer, or a pharmaceutically acceptable salt thereof, has a targeting sequence complementary to a target region comprised in SEQ ID NO: 178. In a further embodiment, the target region is H9A(−5+20) or H9A(+1+25). In still a further embodiment, the antisense oligomer, or a pharmaceutically acceptable salt thereof, has a targeting sequence comprising a sequence selected from SEQ ID NOs: 121 and 122.

In an embodiment, the antisense oligomer, or a pharmaceutically acceptable salt thereof, has a target sequence complementary to the target region H9A(+51+75). In some embodiments, the target region is H9A(+51+75). In some embodiments, the antisense oligomer, or a pharmaceutically acceptable salt thereof, has a targeting sequence comprising SEQ ID NO: 123.

In another embodiment, the antisense oligomer, or a pharmaceutically acceptable salt thereof, has a target sequence complementary to the target region H9D(+7-18). In some embodiments, the target region is H9D(+7-18). In some embodiments, the antisense oligomer, or a pharmaceutically acceptable salt thereof, has a targeting sequence comprising SEQ ID NO: 124.

In an embodiment, the antisense oligomer of Formula (III) and/or Formula (IV), or a pharmaceutically acceptable salt thereof, has a targeting sequence complementary to a region within an intron/exon junction or an exon internal junction of the human UMOD gene pre-mRNA, wherein the targeting sequence comprises a sequence selected from:

| SEQ ID NO: 32 | |
|---|---|
| | (GGGAGATATCTGAAACAGGT); |
| SEQ ID NO: 37 | |
| | (AAGACCTTGTCGAAGCCCAGACTCT); |
| SEQ ID NO: 62 | |
| | (TTTGATGTTGAGGTCACGGA); |
| SEQ ID NO: 70 | |
| | (GCGGTCTTCAGGCTGACTTT); |
| SEQ ID NO: 74 | |
| | (GGCTGTAGGGCGGTCTTCAG); |
| SEQ ID NO: 81 | |
| | (CCACACCTGACCATTGGCTG); |
| SEQ ID NO: 114 | |
| | (TTCATGGTGTCACAGAGATA); |
| | and |
| SEQ ID NO: 120 | |
| | (GAGTCAACTCACAGGCTTGCACTTT). |

In an embodiment, the antisense oligomer of Formula (III) and/or Formula (IV), or a pharmaceutically acceptable salt thereof, has a targeting sequence that comprises or consists of any one of the sequences:

| SEQ ID NO: 7 | |
|---|---|
| | (CTGTGAACAGAGATGGATGGGACAA); |
| SEQ ID NO: 8 | |
| | (TCCTGTGAACAGAGATGGAT); |
| SEQ ID NO: 9 | |
| | (GTCCTGTGAACAGAGATGGA); |
| SEQ ID NO: 10 | |
| | (TGTCCTGTGAACAGAGATGG); |
| SEQ ID NO: 11 | |
| | (GTCTGGTGTCCTGTGAACAGAGATG); |
| SEQ ID NO: 12 | |
| | (CTCTCTGTCTCTGATGTCTGGTGTC); |
| SEQ ID NO: 13 | |
| | (AGACGGGTTGGCCCTTTGAATTTTT); |
| SEQ ID NO: 14 | |
| | (TCTAGATAGCACCTGCCCAAAGGAA); |
| SEQ ID NO: 15 | |
| | (ATCCTTTCTGCTCTTCCCGC) |
| SEQ ID NO: 16 | |
| | (CATCCTTTCTGCTCTTCCCG); |
| SEQ ID NO: 17 | |
| | (AGAGATGGCTGCCCCATCCTTTCTG); |
| SEQ ID NO: 18 | |
| | (ATGGCTGCCCCATCCTTTCT); |
| SEQ ID NO: 19 | |
| | (AGATGGCTGCCCCATCCTTT); |
| SEQ ID NO: 20 | |
| | (CAAGTCAGAGATGGCTGCCCCATCC); |
| SEQ ID NO: 21 | |
| | (TCCAAGTCAGAGATGGCTGC); |
| SEQ ID NO: 22 | |
| | (ATCCAAGTCAGAGATGGCTG); |
| SEQ ID NO: 23 | |
| | (CATCCAAGTCAGAGATGGCT); |
| SEQ ID NO: 24 | |
| | (CAGCATCCAAGTCAGAGATG); |
| SEQ ID NO: 25 | |
| | (TCAGCATCCAAGTCAGAGAT); |
| SEQ ID NO: 26 | |
| | (ACCATCAGCATCCAAGTCAG); |
| SEQ ID NO: 27 | |
| | (AGAGGCCACCACCACCATCAGCATC); |
| SEQ ID NO: 28 | |
| | (CAGTGGCTGCAGTTGTGATGAACCA); |
| SEQ ID NO: 29 | |
| | (TTTCACTTACTTGCTTCTGAGGTGT); |
| SEQ ID NO: 30 | |
| | (GATATCTGAAACAGGTTAGG); |
| SEQ ID NO: 31 | |
| | (AGATATCTGAAACAGGTTAG); |
| SEQ ID NO: 32 | |
| | (GGGAGATATCTGAAACAGGT); |
| SEQ ID NO: 33 | |
| | (AGGGAGATATCTGAAACAGG); |
| SEQ ID NO: 34 | |
| | (CACTTGCCCAGCGACACC); |
| SEQ ID NO: 35 | |
| | (TCAGCTGGCACTTGCCCAGCGACAC); |
| SEQ ID NO: 36 | |
| | (GGCACTTGCCCAGCGACA); |
| SEQ ID NO: 37 | |
| | (AAGACCTTGTCGAAGCCCAGACTCT); |
| SEQ ID NO: 38 | |
| | (CTTGTCGAAGCCCAGACT); |
| SEQ ID NO: 39 | |
| | (ACCTTGTCGAAGCCCAGA); |
| SEQ ID NO: 40 | |
| | (AGACCTTGTCGAAGCCCA); |
| SEQ ID NO: 41 | |
| | (TGAAGACCTTGTCGAAGC); |
| SEQ ID NO: 42 | |
| | (ATGAAGACCTTGTCGAAG); |
| SEQ ID NO: 43 | |
| | (GGTACATGAAGACCTTGT); |
| SEQ ID NO: 44 | |
| | (GGTTGTCTCTGTCATTGAAGCCCGA); |
| SEQ ID NO: 45 | |
| | (TCACTACAGACACCCAGTCC); |
| SEQ ID NO: 46 | |
| | (GTCACTACAGACACCCAGTC); |
| SEQ ID NO: 47 | |
| | (GGTCACTACAGACACCCAGT); |
| SEQ ID NO: 48 | |
| | (ACCGTCAACACTGTCCCACA); |
| SEQ ID NO: 49 | |
| | (TACCGTCAACACTGTCCCAC); |
| SEQ ID NO: 50 | |
| | (GTACCGTCAACACTGTCCCA); |
| SEQ ID NO: 51 | |
| | (ACGTACCGTCAACACTGTCC); |
| SEQ ID NO: 52 | |
| | (GACGTACCGTCAACACTGTC); |
| SEQ ID NO: 53 | |
| | (GGACGTACCGTCAACACTGT); |
| SEQ ID NO: 54 | |
| | (AGGACGTACCGTCAACACTG); |
| SEQ ID NO: 55 | |
| | (CAGGACGTACCGTCAACACT); |
| SEQ ID NO: 56 | |
| | (TGTAAGTGGCATGGGTTTCATTCCT); |
| SEQ ID NO: 57 | |
| | (TCATCTGCCAGGTAGAGGGTGTTGC); |
| SEQ ID NO: 58 | |
| | (GGTCACGGATGATGATCTCA); |
| SEQ ID NO: 59 | |
| | (AGGTCACGGATGATGATCTC); |
| SEQ ID NO: 60 | |
| | (GAGGTCACGGATGATGATCT); |
| SEQ ID NO: 61 | |
| | (TTGATGTTGAGGTCACGGAT); |
| SEQ ID NO: 62 | |
| | (TTTGATGTTGAGGTCACGGA); |
| SEQ ID NO: 63 | |
| | (TTTTGATGTTGAGGTCACGG); |
| SEQ ID NO: 64 | |
| | (GGTAGGAGCATGCAAAGTTGATTTT); |
| SEQ ID NO: 65 | |
| | (TAGGAGCATGCAAAGTTGAT); |
| SEQ ID NO: 66 | |
| | (TTCAGGCTGACTTTCATGTCCAGGG); |
| SEQ ID NO: 67 | |
| | (GCTGACTTTCATGTCCAGGG); |
| SEQ ID NO: 68 | |
| | (GGTCTTCAGGCTGACTTTCA); |
| SEQ ID NO: 69 | |
| | (CGGTCTTCAGGCTGACTTTC); |
| SEQ ID NO: 70 | |
| | (GCGGTCTTCAGGCTGACTTT); |
| SEQ ID NO: 71 | |
| | (GGCGGTCTTCAGGCTGACTT); |
| SEQ ID NO: 72 | |
| | (CTGTAGGGCGGTCTTCAGGC); |
| SEQ ID NO: 73 | |
| | (GCTGTAGGGCGGTCTTCAGG); |
| SEQ ID NO: 74 | |
| | (GGCTGTAGGGCGGTCTTCAG); |
| SEQ ID NO: 75 | |
| | (TGGCTGTAGGGCGGTCTTCA); |
| SEQ ID NO: 76 | |
| | (TTGGCTGTAGGGCGGTCTTC); |
| SEQ ID NO: 77 | |
| | (ATTGGCTGTAGGGCGGTCTT); |
| SEQ ID NO: 78 | |
| | (CTGACCATTGGCTGTAGGGC); |
| SEQ ID NO: 79 | |
| | (CCTGACCATTGGCTGTAGGGCGGTC); |
| SEQ ID NO: 80 | |
| | (CACACCTGACCATTGGCTGT); |
| SEQ ID NO: 81 | |
| | (CCACACCTGACCATTGGCTG); |
| SEQ ID NO: 82 | |
| | (TTGAGTCTCTAGTGTGTGGGCATCT); |
| SEQ ID NO: 83 | |
| | (ACTCCCCATTCTCCACCACTTGGAT); |
| SEQ ID NO: 84 | |
| | (TGGACGGAAAATCGGCCCTGGGAGG); |
| SEQ ID NO: 85 | |
| | (CATCTGGACGGAAAATCGGC); |
| SEQ ID NO: 86 | |
| | (ACATCTGGACGGAAAATCGG); |
| SEQ ID NO: 87 | |
| | (AACATCTGGACGGAAAATCG); |
| SEQ ID NO: 88 | |
| | (GAACATCTGGACGGAAAATC); |
| SEQ ID NO: 89 | |
| | (AACCGGAACATCTGGACGGA); |
| SEQ ID NO: 90 | |
| | (AAACCGGAACATCTGGACGG); |
| SEQ ID NO: 91 | |
| | (CAAACCGGAACATCTGGACG); |
| SEQ ID NO: 92 | |
| | (TTCCAGCAAACCGGAACATC); |
| SEQ ID NO: 93 | |
| | (ATAGTTTCCAGCAAACCGGAACATC); |
| SEQ ID NO: 94 | |
| | (TTTCCAGCAAACCGGAACAT); |
| SEQ ID NO: 95 | |
| | (GTTTCCAGCAAACCGGAACA); |
| SEQ ID NO: 96 | |
| | (AGTTTCCAGCAAACCGGAAC); |
| SEQ ID NO: 97 | |
| | (TAGTTTCCAGCAAACCGGAA); |
| SEQ ID NO: 98 | |
| | (ATAGTTTCCAGCAAACCGGA); |
| SEQ ID NO: 99 | |
| | (CATAGTTTCCAGCAAACCGG); |
| SEQ ID NO: 100 | |
| | (TCATAGTTTCCAGCAAACCG); |
| SEQ ID NO: 101 | |
| | (AGGTCATAGTTTCCAGCAAA); |
| SEQ ID NO: 102 | |
| | (GGTAGACTAGGTCATAGTTT); |
| SEQ ID NO: 103 | |
| | (AGGTAGACTAGGTCATAGTT); |
| SEQ ID NO: 104 | |
| | (CAGGTAGACTAGGTCATAGT); |
| SEQ ID NO: 105 | |
| | (CTTCACAGTGCAGGTAGACTAGGTC); |
| SEQ ID NO: 106 | |
| | (ACAGTGCAGGTAGACTAGGT); |
| SEQ ID NO: 107 | |
| | (CACAGTGCAGGTAGACTAGG); |
| SEQ ID NO: 108 | |
| | (TCACAGTGCAGGTAGACTAG); |
| SEQ ID NO: 109 | |
| | (TTCACAGTGCAGGTAGACTA); |
| SEQ ID NO: 110 | |
| | (GTCACAGAGATAGACTTCAC); |
| SEQ ID NO: 111 | |
| | (TGTCACAGAGATAGACTTCA); |
| SEQ ID NO: 112 | |
| | (GTGTCACAGAGATAGACTTC); |
| SEQ ID NO: 113 | |
| | (TCATTCATGGTGTCACAGAGATAGA); |
| SEQ ID NO: 114 | |
| | (TTCATGGTGTCACAGAGATA); |
| SEQ ID NO: 115 | |
| | (ATTCATGGTGTCACAGAGAT); |
| SEQ ID NO: 116 | |
| | (TTTCATTCATGGTGTCACAG); |
| SEQ ID NO: 117 | |
| | (TTTTCATTCATGGTGTCACA); |
| SEQ ID NO: 118 | |
| | (TGCACTTTTCATTCATGGTG); |
| SEQ ID NO: 119 | |
| | (AGGCTTGCACTTTTCATTCA); |
| SEQ ID NO: 120 | |
| | (GAGTCAACTCACAGGCTTGCACTTT); |
| SEQ ID NO: 121 | |
| | (AATCTGGTCCCAGAGCAGGTCTACA); |
| SEQ ID NO: 122 | |
| | (TTCGGAATCTGGTCCCAGAGCAGGT); |
| SEQ ID NO: 123 | |
| | (TGTGATGGGACCCAAGTTCAGGACA); |
| SEQ ID NO: 124 | |
| | (AGCGAGTGGCTCTCTTACCTTTCCG); |
| | and |
| SEQ ID NO: 125 | |
| | (GAGGGAGATATCTGAAACAG). |

In an embodiment, the antisense oligomer, or a pharmaceutically acceptable salt thereof, has a targeting sequence complementary to a target region within the 5^{th} nucleotide of intron 8 measured from the 5′ end of exon 9 and the 25^{th} nucleotide of exon 9 measured from the 5′ end of exon 9 of the human UMOD gene pre-mRNA. In certain embodiments, the antisense oligomer, or a pharmaceutically acceptable salt thereof, has a targeting sequence complementary to a target region comprised in SEQ ID NO: 178. In a further embodiment, the target region is H9A(−5+20) or H9A(+1+25). In still a further embodiment, the antisense oligomer, or a pharmaceutically acceptable salt thereof, has a targeting sequence comprising a sequence selected from SEQ ID NOs: 121 and 122.

In an embodiment, the antisense oligomer, or a pharmaceutically acceptable salt thereof, has a target sequence complementary to the target region H9A(+51+75). In some embodiments, the target region is H9A(+51+75). In some embodiments, the antisense oligomer, or a pharmaceutically acceptable salt thereof, has a targeting sequence comprising SEQ ID NO: 123.

In another embodiment, the antisense oligomer, or a pharmaceutically acceptable salt thereof, has a target sequence complementary to the target region H9D(+7-18). In some embodiments, the target region is H9D(+7-18). In some embodiments, the antisense oligomer, or a pharmaceutically acceptable salt thereof, has a targeting sequence comprising SEQ ID NO: 124.

In some embodiments, the antisense oligomer of Formula (I) is an antisense oligomer selected from:

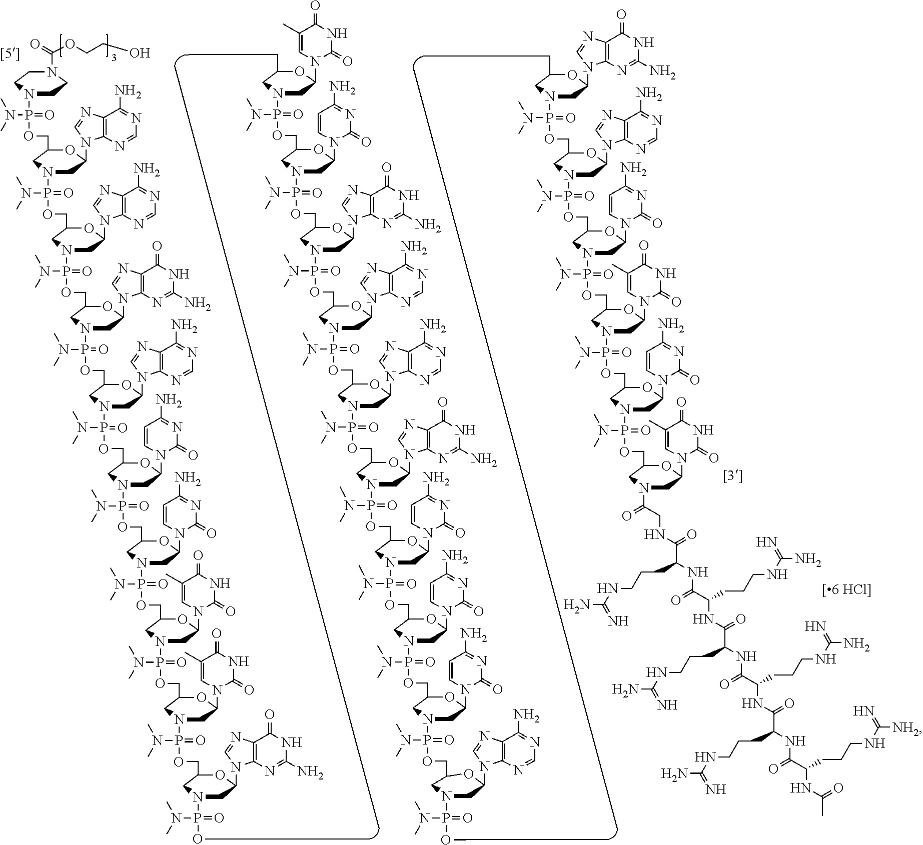

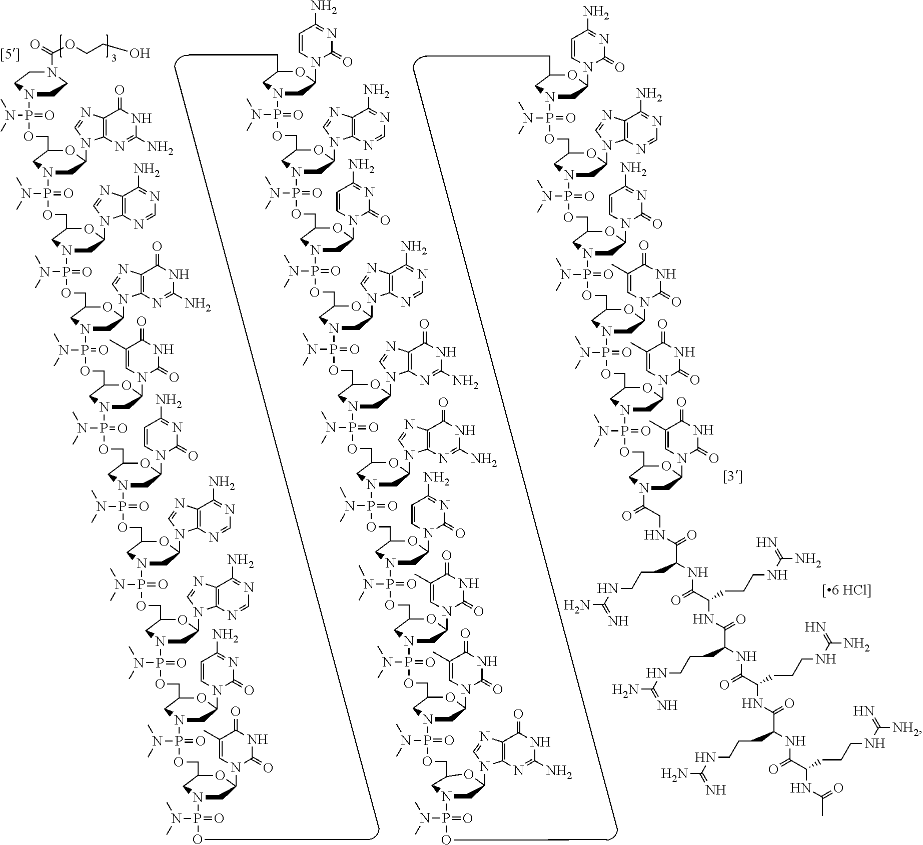

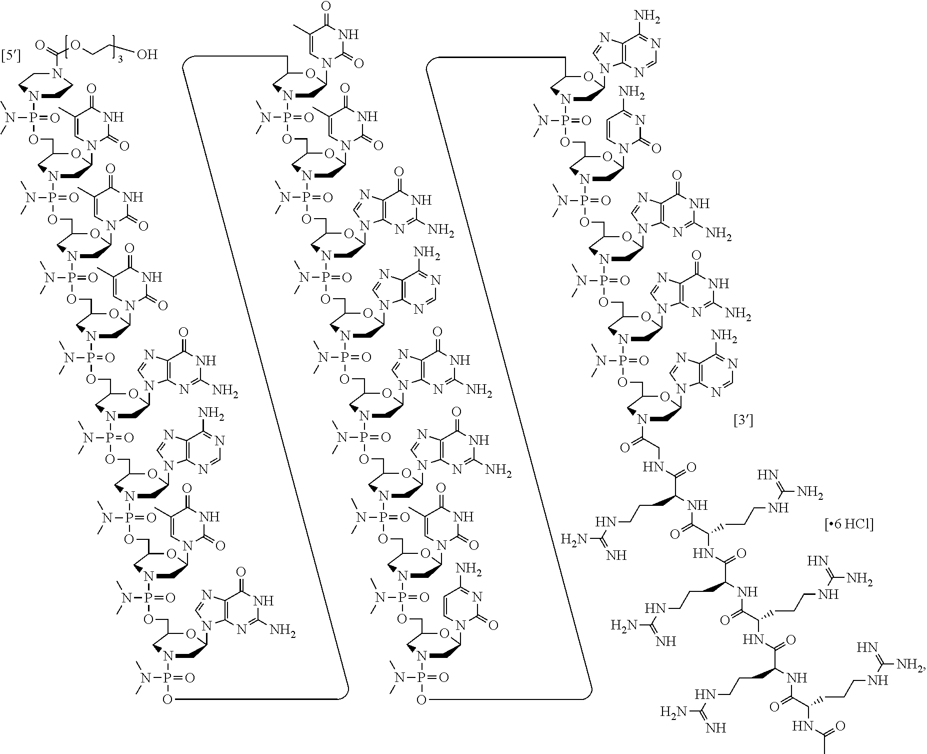

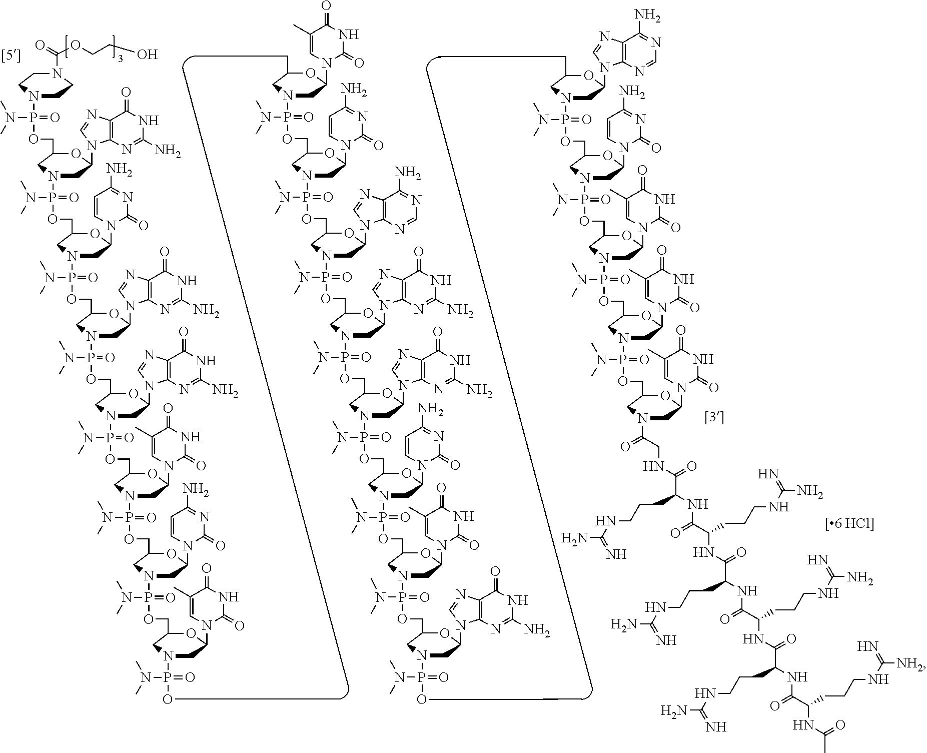

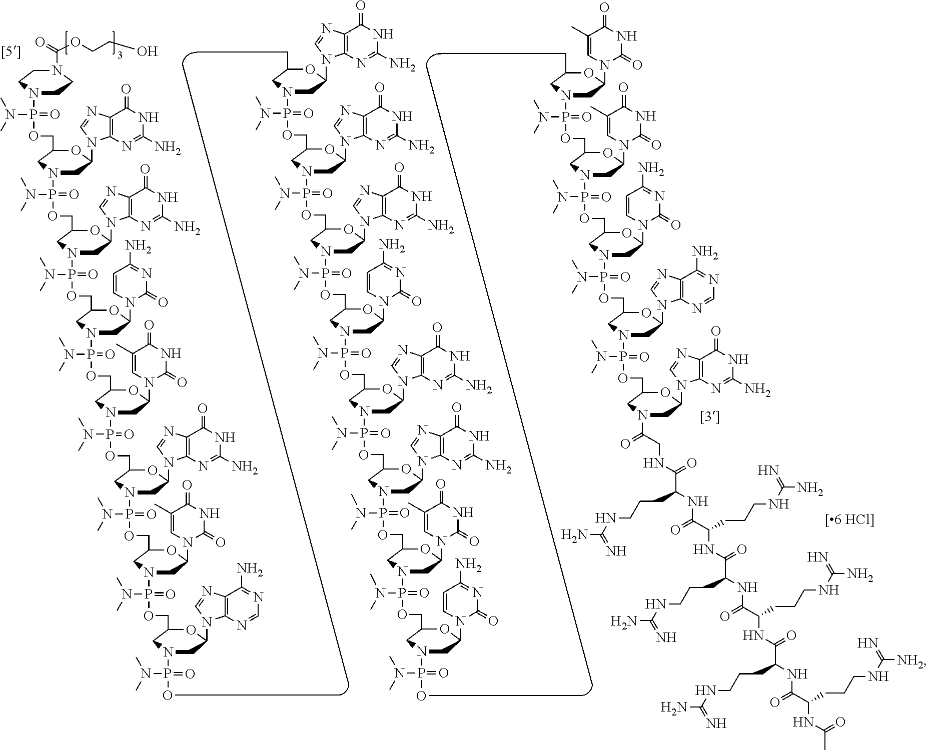

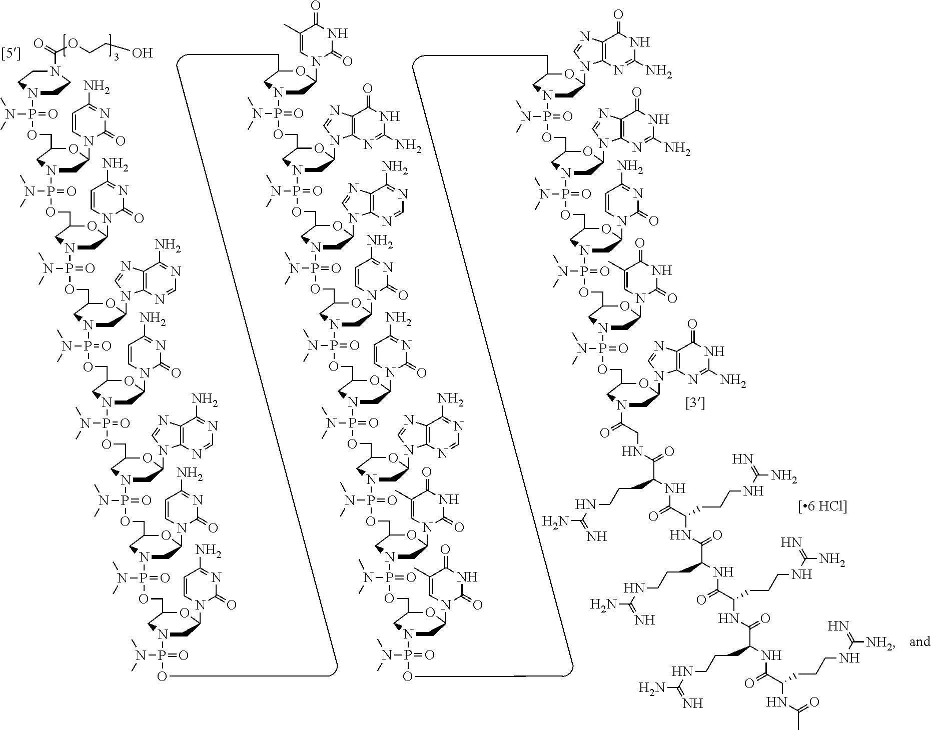

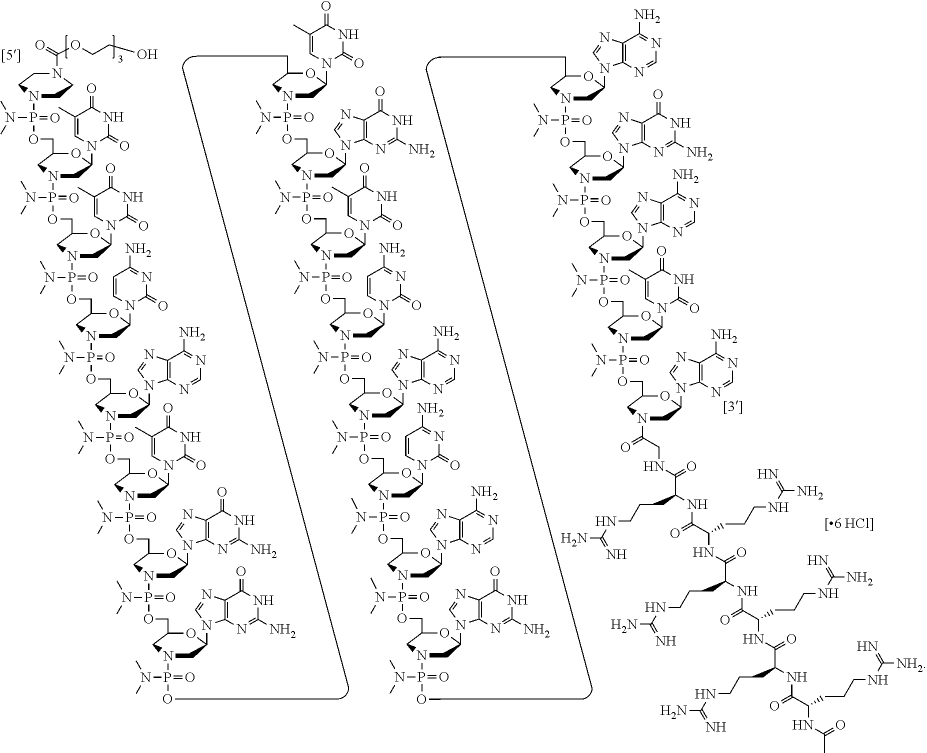

### IV. Target Sequences and Target Regions

In some embodiments for antisense applications, the oligomer can be 100% complementary to the nucleic acid target sequence (excluding at least one abasic subunit), or it may include mismatches, e.g., to accommodate variants, provided that a heteroduplex formed between the oligomer and nucleic acid target sequence is sufficiently stable to withstand the action of cellular nucleases and other modes of degradation which may occur in vivo. Mismatches, if present, are less destabilizing toward the end regions of the hybrid duplex than in the middle. The number of mismatches allowed will depend on the length of the oligomer, the percentage of G: C base pairs in the duplex, and the position of the mismatch(es) in the duplex, according to well-understood principles of duplex stability. Although such an antisense oligomer, or a pharmaceutically acceptable salt thereof, is not necessarily 100% complementary to the nucleic acid target sequence, it is effective to stably and specifically bind to the target sequence, such that a biological activity of the nucleic acid target, e.g., expression of the encoded protein(s), is modulated.

The stability of the duplex formed between an oligomer and the target sequence is a function of the binding Tₘ and the susceptibility of the duplex to cellular enzymatic cleavage. The Tₘ of an antisense compound with respect to complementary-sequence RNA may be measured by conventional methods, such as those described by Hames et al., Nucleic Acid Hybridization, IRL Press, 1985, pp. 107-108 or as described in Miyada CG. and Wallace RB (1987) Oligonucleotide hybridization techniques, Methods Enzymol. Vol. 154 pp. 94-107.

In some embodiments, each antisense oligomer, or a pharmaceutically acceptable salt thereof, has a binding Tₘ, with respect to a complementary-sequence RNA, of greater than body temperature or in other embodiments greater than 50° C. In other embodiments, Tₘs are in the range of 60-80° C. or greater. According to well-known principles, the Tₘ of an oligomer compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. In certain embodiments, compounds show high Tₘ (50° C. or greater) at a length of 20 bases or less. For some applications, longer oligomers, for example longer than 20 bases, may have certain advantages.

The targeting sequence bases may be naturally occurring DNA bases or analogs thereof, e.g., uracil and inosine that are capable of Watson-Crick base pairing to target-sequence RNA bases.

An antisense oligomer, or a pharmaceutically acceptable salt thereof, can be designed to block or inhibit or modulate translation of mRNA or to inhibit or modulate pre-mRNA splice processing, or induce degradation of targeted mRNAs, and may be said to be “directed to” or “targeted against” a target sequence with which it hybridizes. In certain embodiments, the target sequence includes a region including a 3′ or 5′ splice site of a pre-processed mRNA, a branch point, or other sequence involved in the regulation of splicing. The target sequence may be within an exon or within an intron or spanning an intron/exon junction.

An antisense oligomer, or a pharmaceutically acceptable salt thereof, having a sufficient sequence complementarity to a target RNA sequence to modulate splicing of the target RNA means that the antisense agent has a sequence sufficient to trigger the masking of a binding site for a native protein that would otherwise modulate splicing and/or alters the three-dimensional structure of the targeted RNA. Likewise, an oligomer reagent having a sufficient sequence complementary to a target RNA sequence to modulate splicing of the target RNA means that the oligomer reagent has a sequence sufficient to trigger the masking of a binding site for a native protein that would otherwise modulate splicing and/or alters the three-dimensional structure of the targeted RNA.

In certain embodiments, the antisense oligomer, or a pharmaceutically acceptable salt thereof, has sufficient length and complementarity to target region within a pre-mRNA of the human UMOD gene (SEQ ID NO: 1). The human UMOD gene (SEQ ID NO:1) and the exon 2 (SEQ ID NO: 2), exon 5 (SEQ ID NO: 3), exon 6 (SEQ ID NO: 4), exon 8 (SEQ ID NO: 5), and exon 9 (SEQ ID NO: 6) sequences for human UMOD gene pre-mRNA are shown in Table 2.

TABLE 2
Target Region for UMOD-targeted oligomers

| | | SEQ |
|---|---|---|
| Name | Sequence (5′-3′) | ID NO |
| Human | AGATCATGTGGCATAGAGCCCCTGACAGGCCACGAGCCAGAGAATG | 1 |
| UMOD gene | GGTGTGCCACCTCCAGGTCCCCCAGGCGTAACTGGAGCCTCAGGAG | |
| | GTGTGTGCTCAGAGAGGTAGGTGGGACAGGTAATACCCCTCTTTTG | |
| | GGGGATCTTCTCCCTTGGCTCATTTTTTGCCGCTTTCTGACACACA | |
| | CCAACCTCTCATTCTAAGTTTTTGTGTTCTGTTTGATCAATTTTTC | |
| | AAAACTCTCCACCCTTTAGTTTCACTCTTTCCAAAACTACCTAAAA | |
| | CCACTTATCCTTCTAAGACCCTGTCATGGCTGAGCATGGAATGTTC | |
| | TGTAACCCAAGGAATGCAGGTAAATCTGAGTGAGATTTGAGTTTTA | |
| | GTCTGGATTTAACACTCGCTAGCTGTGTAATCTTGGGGGAGTTACT | |
| | CCACCTCTCTGAACTTGTCTTCTTCCCTGTGACGTGGGTTGTTGTG | |
| | TTTCATTGTTTGAGACAATGGAGGCAAAAGCACTGAGCACAGTGTC | |
| | TGACCCAAGAGAAGTGCTCAGTGGGTGATTGTTCTCAGCATTATTA | |
| | AGATCAAGTCTCCTACCCATGTTTGAAAGTTAAGAGAGAAAAGTGT | |
| | TTCAGGCTGGTTGTGGTGGCTCATGTCTGTAATCCCAGCACTTTGG | |
| | GAGGCCGAGGCAGGCAGATTACCTGAGGTCAGGAGTTCGAGACCAG | |
| | CCTGGCCAATGTGTACAAAAAAAACACAAATTAGCCGGGCATGGTG | |
| | GTATGCACCTGTAATCCCAGCTACTCGGGAGGCTGAGGATTCAAGG | |
| | ATTCAAGAGAATCACTTGAACTTGGGAGGTGGAGGTTGCAGTGAGC | |
| | CAAGACTGAGCCACTGCACTCCAACCTGGGTGACAGAGCAAGACTC | |
| | CATTAAAAAAAAAAAAAAGAAGAAGAAAAAGAAAGAAAGAAAGAAG | |
| | AGTGTTTCAAGAGCGAAGTGCTGAGAGATTAAGGTTCGTTCAAAAA | |
| | CAAATATTTATTGTGCACTGTCCTAGAGGTAAGATAAAGAGAAAAA | |
| | TATTGATTGAATTTGGTAACATGGATGTCTTTGGTGAACTTGATGA | |
| | GCAACAGAGCAGGAAATTATGGCCACTGACAACTTGTTATACTCAC | |
| | AGATCCCAAGAGAAGGGGACACACCACACTGGGCAGAGGGGCTGAA | |
| | GGGAGAGGAAGAGCTGCTGGGAAGTACCAGGGTCGGTCAGGAAGCA | |
| | AAGAGAGCAAGGGAAAAGGCAGGAAAGAGCCTTTATTGTAGTTTCT | |
| | GCAGGAAGGAATGGGAGAGGCAAGGTAAGCAGATTTAGGATTGGCT | |
| | TAGTTTGAATAATTTCAGAGACCTCTGGGCCATAGGAGTTGTCTCT | |
| | AGTTGTCTGGTACCTGGTCCCTTGGGGTGATCTAAAGCAGCAAGTA | |
| | GTGACCCTAAGTGTAAAAGCCCCAAAAAGGAGGCCAAGGGTTGGGG | |
| | GTATGTGGTCTGGATTGATTGATTTGCAGTCGGAAAGGCACATTCA | |
| | CAGATAAGTCGTTTTTGATCTCTAGTAATTATTGAGCCCTGGGAGG | |
| | GGCAGTCCCTCCATGATCAGTAAGGCCCCAAAATGTACAAGCACCA | |
| | AATACAGAAACTAGAAAGCATGGTGATTACACTTATGAATAGATTC | |
| | CAACCCAAACTCCCCATTTTCCTGCCCAAGTCTTTGTCAAGAAAAC | |
| | TCCAGAAGCCACCTGGGGTCACTGTCTTTAATCCTTTGAAGACACC | |
| | CATAATAAGGTTTAGACAAGGCCTCATGAAAAAGGTATAGGATAAA | |
| | AACGACTAGTTCAGAAAAAAAAAAGTAAATAAATGAAGAGATAACT | |
| | TCCCTTTTCATCAAAGAATTGCAAAAATGAACAATAATGAGATCGT | |
| | TTCCACTTATCCTTTTGGCAAAGATGAAAGCAAGTTACCAATGCTC | |
| | AGAACTGGTGAGTAGTGTTGGTGGGATTGTAAACCAATTTATAATA | |
| | ATTCTCTGGGGTGCTGTTTGGGAAGAGGAGTCAATATTCCTACAGC | |
| | TGTGCTACCTCTTTGACCCAGAATTTCCACTCCTTGGACATAATCC | |
| | TGAGGAGATGACAACACCAGTGCCAGTGGTTACCCTACCACTGAGT | |
| | GGGACAGGATGGAGAAATGTCCTGAAAAGAGCAATTCACAATAGCA | |
| | AAGACTTGGAACCAACTCAAATGTCCATCAATGATAGACTGGATTA | |
| | AGAAAATGTGGCACATATACACCGTGGAATACTATGCAGCCATAAA | |
| | AAATATGAGTTCATGTCCTTTGTAGGGACATGGATGAAGATGGAAA | |
| | CCATCATTCTCAGCAAACTATCGCAAGGACAAAAAACCAAACACCG | |
| | CATGTTCTCACTCACAGGTGGGAATTGAACAATGAGAGCACTTGGA | |
| | CACAGGAAGGGGAACATCACACCCAGGGGCCTATTGTGGGATGCGG | |
| | GGAGCGGGGAGGGATAGCATTAGGAGTTATACCTAATGTAAATGAC | |
| | AAGTTAATGGGTGCAGCACATCAACATGGCACATGTATACATATGT | |
| | AACAAACCTGCACGTTGTGCACTTGTACCCTAAAACTTAAAGTATA | |
| | AAAAAAAAAAAGATCAATGCAGTGATCATGGTGATATTTTCCTGCT | |
| | CAGCCCAAGTTCACACATATTTTATTTTTCTCAACATGATGACAGC | |
| | CACTCTCACACTGACTTTTGGAATGTCATGTATGTTGAACTGGGTC | |
| | TGAAGACATGGTTTTAACTCAGGCTCTGTCATTTTCTACCTCAGTG | |
| | ATTGCACAACAGCAAAGCAGAATTTTCACTACTTCCATGAATATAA | |
| | TCATTACTATATGACTTTACTTGCATCATCTCCTTTGGTTACTATT | |
| | ACTACTGTGGGAGATGGGTATTCTCATTTTATAGACAAGGAAATTG | |
| | ACCTCTGGACCTCAGGAAGGTTAAGAAATGAGCCCACTGCCACACA | |
| | ATAAACACCAGATAAAGGAGGCAGACTGACTCCAAAGTCAGTCTAT | |
| | TTAAGTGCAAATTTATTTCGCCTCCAAAGGGACCTCCCAGTCATCA | |
| | GACCTGATTCTTTGTTGTACAGAGTGGGTCAGGTCCAGTGATGTCT | |
| | GAACTACCTTCTGGTTCTGACTTTCAGCCATTCTCAGCTCCTCTCT | |
| | TGCTTGTGTCTGGATTCTAAGGCTGATCTCATGAGAATGGGTGTTT | |
| | CAGAAGGGTGCCCTCTCCAAGACAGGTGCACCTCCCATCTGGGGCA | |
| | GTGAATATCCCTTTTGTCCTTATGCAGCCTGGCTTCAGATACTGGC | |
| | TTCTGCCTGGCTCCTTGATCCCACCCTGCCCTTGTCAGTGACCAAG | |
| | AAGAAGCCCAGCACCTTGGCACTGCTTTCCCAGTTAATTTCTAACT | |
| | ATGGAATCTCTTGCTGTTAGAAGGTGCGAAACAGTGACCTTGTATT | |
| | TCCGGGCACAGGTGTGACCCCCCAATGTCAATCATTTGGGGTCTCT | |
| | AGCTATTAGGAAAAAGAACAACAACAACCTCACAGCTTGGACAAGG | |
| | CAAACATTATGCCAGGAGGAAAAAATATTCCACCCCCAAGAAAACA | |
| | ATATCAAAAAACAGAACTAGAGACTAATTGGAGGAGAGATTGCCAG | |
| | CCTGGGGCAAATGTGTATATATAAGTATGAGGCACATCATGACCAG | |
| | ACTAACTCTACCTTTCTGGCTTCAGGTAAGGCTATCTGTAGCTGTC | |
| | TTCTCCTAGCCCAGCTTCTCCCCATCCTATTTGAGGGAGGTAGGAG | |
| | AGGAAATTAAGACCTTGGACACTGGGGTCAGACCTGGATTTGAGTC | |
| | ATTATTCTGCCAATTATTGGCTTCATGATCTTTAGTAAGTTAGTTT | |
| | TCCTCTCTTTGATCCTCTCTGTCCTAGTAACAATGACTACTATTTT | |
| | TTGACTTATTCCATGAATATTAGTTATGCACCTATTATGTGCCAGG | |
| | TACCAGGGTTACAACAATGAACAACTTGCTATGTGCTGGGCCCTGC | |
| | TCGACATATGTCATCTTAGTTAATCCCAGTGACAATATGCAGTGTT | |
| | TTAGGTCGCATCTTATTAGCGGGTCATAAAATCATTCCTTTTGTGG | |
| | GTTATATCCAGGGACTTCTTTTAATTAAATATAATAGAATATGTCA | |
| | GTAAGCAATACAAATTTGAGGCAAGCAGTTAGCCTCTTTGTCCCCC | |
| | TGTTTTCTCATCTGCAAACTAGGATAATACTTATCTCATAGGGTTG | |
| | TGGTAAGAATTAAATCACTTAATGTAGTAAGGATATCAATTGTCTT | |
| | ATAAAATTTTATGTGTGTGTGCACATACATGCAGAAGCGACTGCAC | |
| | TAGATCATGACATAAAATGTTTTTCTTTCTTTCTGTCTTTTTTTTT | |
| | TTGGGGGGGGGGGATGGACTCTCTGTCACCCAGGCAGGAGTGCAGT | |
| | GGCACGATCTCAGCTCACTGCAACCTCTGCCTCCCAGGTTCAAGCA | |
| | ATCCTCCTGTCTCAGCCTCCCGAGTAGCTGGGATTACAGGCGCCCA | |
| | CCACCATGCCCGGCTAATTTTTGTATTTTTATTAGAGATGGGGTTT | |
| | CACCATGCTGGCCAGGCTGGTCTCAAACTCCTGACCTTGTGATCCA | |
| | CCCCCTCAGTCTCCCAAAGTGTTGGGATTACAGGCATGAGCCACTG | |
| | CACCTGGCAACATAAAATGTTTTTCTTTTAGTACATCATGGTCACG | |
| | AAGATCTGGAAAACGTTGCTCTAGGACCCAATGCCTTTGAGCACAT | |
| | GAATGTCTTTGAACTGAGCCTGGCCCATCATAGATATTCAGTTTGT | |
| | GAAAGCAACAATCTCCATTTCACAGACAAAAACGGAGACTTAGTAA | |
| | ACTGAAATACCCACATCACAGTGTTAGTGATCAGTAAAGCAGGGAT | |
| | TTGAACCTCAGGTGCAGGCTGCAGCTCCTACATACTTAACCAACAT | |
| | GCCACCCTATTTTGCTGCTATAAACACTGGGGTAATACTGGCATCC | |
| | ACCCTGGACTATTTTTCCAATTGGAAGAAATAGTAAGTGACCACTC | |
| | CAGAAACATTTAAATGCATTGTGAAGGTTAATTACCTCTCAAGTTG | |
| | CTCGTCTCAAGCCACTTCTTTACGATATGTGAGTTGCATGAAGGAG | |
| | CAGACTGCCATGTTCATTGTTCTCCATGGTGACTAGCTCCCTGTTT | |
| | GGCACAATGTAGCTACTCAATAGATGTTTGTTGAATGAATAAGTGA | |
| | TCCAGGCAAATTGAAAGTTTCCAGGCAGAGGATCAGTTAAAATCCA | |
| | AAGCTGCCTTCAGTAAAGTGGTGAGTCCAGAACACTATTCCATCTG | |
| | GCTACTTCCTGCTCCAAATGACTGAGTTCTTCAAAATGTGCAATGT | |
| | GCTGAGAATTGGGGAGCCAAGACTGGGATGTTGGTGAGGTAAGGAG | |
| | GGGGAGTACAAGGGGTAAAGTCCCAGCAAAACAAGGGCTGCAGTGT | |
| | TATGCAATTTTTTAGTCCATATAAGTGACACCTCCTGGAGTTGTAT | |
| | ACTATACAATCAAAGCACTCCTTCCAGCTGTGGGGAGGAGAGTTAG | |
| | ATCATGCATTTGTCCCATCCATCTCTGTTCACAGGACACCAGACAT | |
| | CAGAGACAGAGAGAAAAATTCAAAGGGCCAACCCGTCTTTCCTTTG | |
| | GGCAGGTGCTATCTAGACCTGAAGTAGCGGGAAGAGCAGAAAGGAT | |
| | GGGGCAGCCATCTCTGACTTGGATGCTGATGGTGGTGGTGGCCTCT | |
| | TGGTTCATCACAACTGCAGCCACTGACACCTCAGAAGCAAGTAAGT | |
| | GAAAAGTGTGTGTGCGTTGTATATGTGTGTATGCACGTGTATGTGT | |
| | GAATGTGTGGGGGAAGCAATGTAGCACCTGTCAGAGGTGATCTCAA | |
| | TCCTCCTATCGCACTTGAGACTTGCATTGTCTTCATTCTAAGTCCA | |
| | TTTCTTAGACTCAATATGCACAGGACTGACTTAGAAATTTTGCTAA | |
| | AGTGCATATTCTGGTTCAGCAGATCTGCAGTAGGACCTGAGATGCT | |
| | GAATTTTTAACAAGCTTCCAGGCGATGCTCATACTGGGGTCCCTGG | |
| | AGTACACATTGAAAAGCAAGGGGCTAGAACATCTCTAAGGCCTGCA | |
| | GGCCCTTTATTGGAAGTCAGAAACATACTCTATCACATAGGAGATT | |
| | TGAACCCATGCAGGAGGATCCAAACACCTTCCCTTTCAACTTTAAG | |
| | AGGTCATTCCATTGGGTTGAGATTTGCTGTCACCCCACTTTCATTT | |
| | TCTCCCTGGAGTACGTTGGGGCACGATGAATACTATTGCGGTGTCC | |
| | TGGTTAAAAGCACATATTTTGTGGTCCTGCCATCTGCGTTTTTATC | |
| | CTGGTTCTACTTCTTACCAAAGGAGTAAGGGGCTTAATCCCTCTGA | |
| | ACCTCAGTCTCCTCATCTTTAAAATAAGGATACATAAAAAACTGAC | |
| | CTCACGAGGCCCTTGGGAAGTATTCAACAAGATAGTGAGTGAAAAG | |
| | TGCACATCCTATTGCCTGGCATATAGTAATTGCTTAATAAACAACA | |
| | GCTTCTTTTTTTTTTTAATGGTTATTTTTATTACGGAGGAACAAAG | |
| | TACAACTGCCCAGCCAGGTGGAGTTGGAGGACTCCGCAGAGAGGAG | |
| | GCGACACTGAGCAGGGTCCTGATGAAGATTTCACCAGCCAGGGAAG | |
| | CAGAAAACATAAAATGTGCAAAGAAAGGGAGGGGCAACAGGTTCAC | |
| | CGTAAATCCTACCAAAGTATAGGAATTCTGCGCATTACTTTTCTGA | |
| | ATGTGGCTATTTTAAAAGAAGACAGCTTGAAAGCAATGCTTAACAC | |
| | AAAAAATGAATGGTGGAGCTGGGCGCGATTGCACGTGCCTGTGGTC | |
| | CCAACTTTTTGGGAAGCTGAGGCAGGGTGCGGTGGTGGCTTGAACT | |
| | CAGGGAGTAGAACGCTTGAACCCAGGAATTGGAGGCTATGGTGAGC | |
| | TATGATCGCACTACTGCACTCCAGCCTGGGCGACAGAGCAAGACCC | |
| | CTCTCCAAAAAAATAAATAAAGTTAAAAAATACAATGAAATAAACA | |
| | CAGAATGAACGGTGGAGGCTTGACATCATCAGAGGAGTTTTGTTTC | |
| | TTTGCTTCTTTCCTTGTTTTGGAGGGAGCCCTCTAGGGAATAAGTC | |
| | TTAAAAATAATGAGTTCCCTGGAGAATGAGGGAAGGATCTCTGGGT | |
| | GGCCATGGGCCCAGCTGCCCAAACCCTGAAGCTGGGCTTTTCTGTC | |
| | CACAGGATGGTGCTCTGAATGTCACAGCAATGCCACCTGCACGGAG | |
| | GATGAGGCCGTTACGACGTGCACCTGTCAGGAGGGCTTCACCGGCG | |
| | ATGGCCTGACCTGCGTGGACCTGGATGAGTGCGCCATTCCTGGAGC | |
| | TCACAACTGCTCCGCCAACAGCAGCTGCGTAAACACGCCAGGCTCC | |
| | TTCTCCTGCGTCTGCCCCGAAGGCTTCCGCCTGTCGCCCGGTCTCG | |
| | GCTGCACAGACGTGGATGAGTGCGCTGAGCCTGGGCTTAGCCACTG | |
| | CCACGCCCTGGCCACATGTGTCAATGTGGTGGGCAGCTACTTGTGC | |
| | GTATGCCCCGCGGGCTACCGGGGGGATGGATGGCACTGTGAGTGCT | |
| | CCCCGGGCTCCTGCGGGCCGGGGTTGGACTGCGTGCCCGAGGGCGA | |
| | CGCGCTCGTGTGCGCGGATCCGTGCCAGGCGCACCGCACCCTGGAC | |
| | GAGTACTGGCGCAGCACCGAGTACGGGGAGGGCTACGCCTGCGACA | |
| | CGGACCTGCGCGGCTGGTACCGCTTCGTGGGCCAGGGCGGTGCGCG | |
| | CATGGCCGAGACCTGCGTGCCAGTCCTGCGCTGCAACACGGCCGCC | |
| | CCCATGTGGCTCAATGGCACGCATCCGTCCAGCGACGAGGGCATCG | |
| | TGAGCCGCAAGGCCTGCGCGCACTGGAGCGGCCACTGCTGCCTGTG | |
| | GGATGCGTCCGTCCAGGTGAAGGCCTGTGCCGGCGGCTACTACGTC | |
| | TACAACCTGACAGCGCCCCCCGAGTGTCACCTGGCGTACTGCACAG | |
| | GTCAGCCGGAGTCTCCCCACAGTCCTCATCCCAGGCCTGGAAAGGC | |
| | ACTGCAGAGGACGGGGGTGCGTCCTTATTGATTGTCTGTCTGTCCC | |
| | TGTGACCCTGCAGACCCCAGCTCCGTGGAGGGGACGTGTGAGGAGT | |
| | GCAGTATAGACGAGGACTGCAAATCGAATAATGGCAGATGGCACTG | |
| | CCAGTGCAAACAGGACTTCAACATCACTGGTGAGGCCAGTGGGGAG | |
| | GAAGCGGGTTGTTGAGAAACCTGTCACTGCCTGGGGGAGGGACACA | |
| | TTCCTCCCCTGTGAGATTGGGGCCATATGGGTATGACGCAGGGGAT | |
| | ATATATCCAACCTGAGTGAAAACAGAAGATCCACTAATACCCATTA | |
| | AAGCCGGCAAGAGGCTCTCTGAGGCTCCCTGAGTCTCCCTTTAGTT | |
| | GACTTCAAAGCTGCCAAAGATTTGGGGACCTCCTCGCACCCAGCCT | |
| | TCTTTCTGAGGCCCACACCACAGTGGGCACCCACGTTGCTGCCATC | |
| | TGGGAGCCAAAGACCATCTGAGCCCTATTCCTGCAGCACCAGAACA | |
| | CAAGCCCGGCCCGGTGAGCAGTCCACCAATCAGCTCAGGCCTGATG | |
| | ACCAGTCCACCAATCAGCTCAGTACTCCGGGCCAGGCACAAGCCTC | |
| | AGATTTCTCATAGGTACAATTATTGAAGATGAAAATAGTATTTTCA | |
| | GACTTCAGTCATGATTTTAGTTACATCCAAGTGCCAACTTTACAAT | |
| | TGGCACCCTGGTTCACTTGCTTTATCCAAGTGTTACCTGTGCCATG | |
| | ACTTTTATTAAGTTGTTTTTCTTCTAATGGACTGAATCTCTTAAGC | |
| | AAATTTGGAATGTTGATGGTCAGGACTTCATCTGTCTGTGGCAAGT | |
| | TATGCTTACCTTAGACAGAAAGTTAACTGTAAAAATAAAAGTTACT | |
| | AAAAAGAAAAGAAGGTTACTAAATTTCAGCAGTTGTGTAATACTTC | |
| | CATAAAACAAATTTTAAAAAATAAATACTAAAAATTAGAAAAATAC | |
| | TTCAATTTTTAAAAAATTAAACAAGAATTCTGGCTGGATGTTGTTT | |
| | CCTGCCCCAAACTCTCAGCCCGAGGCCTGCTCTCTGTTTGAGAGGG | |
| | AAGTTAGTGGGCCGGGCGCAGTGGCTCATGCCTGTAATCCCAGCAC | |
| | TTTGGGAGGCCGCAGCGGGCAGATCACCTGAGGTCAGGAGTTCAAG | |
| | ACCAGCCTGGCCAACATGGCAAAATCCCATCTCTACTAAAAATACA | |
| | AAAACTAGGTGAGTCTGGTGGCAGGTGCCTGTAATTCCAGCTACTC | |
| | GGGAGGCTGAGGCAGGAGAATCACTTGAACCCAGGATGCGGAGGTT | |
| | GCAGTGAGCCAAGATGGCACCTCTGCATTCCAGCCTGGGCTTGTCG | |
| | CCGAAGAGAGAAACTCCGTCTCAAAAAAAAGAGAGAGAGAGGGAAG | |
| | TTAGTGAGTACATGGCATTAAAGACATTAGTGGCAAGCTGGGACTG | |
| | TCTCCTTGATGTGAAAAGGGTAGAAAGTAATTGCAGCTTGCTTGCT | |
| | TTTTCTTTCTTTCTTTCTTTCTTTCTTTCTTTCTCTCTTTCTTTTT | |
| | TTTATTATACTTCACGTTCTAGGGTACATGTGCACAACGTGCAGGT | |
| | TTGTTACATATGTATACATATGTAACTTGCATCATCTCCTTTGGCT | |
| | ACTATTACTACTGTGGGAGATGCGTATTCTCATTTTATAGACAAGG | |
| | AAATTGACCTCTGGACCTCAAGTAATCTGTCTGCCTTGGCCTCCCA | |
| | AAGTGCTAGGATTATAGGCATGAGCCACCGCATCTAGCCTTTTTAT | |
| | TTTTTTAAACGAGTATTCATTGTTATTTAATGCTGGGACATCAAAA | |
| | CCCCCCAAAACCGTCCTGCATTTGGTATATAACCCACATTTAGGGG | |
| | AACCCAAGACTGAGTGGTTGGTCAGTGGATGTATCCATTGATGTCA | |
| | GAGGTCCAATCTTGAGTCCCCATCACATTGGGAGGGGACAGATCAG | |
| | CTCAAGGCTATGCTGAGCACTTCCAGATGGTGGTCAGCCCAGCCAG | |
| | CTGGACCTGGCCCCTGGGGATGTGCTGGGCCCCCAAGCTATAGACA | |
| | CACGTCCTCAATCCCACCTAACCTGTTTCAGATATCTCCCTCCTGG | |
| | AGCACAGGCTGGAATGTGGGGCCAATGACATGAAGGTGTCGCTGGG | |
| | CAAGTGCCAGCTGAAGAGTCTGGGCTTCGACAAGGTCTTCATGTAC | |
| | CTGAGTGACAGCCGGTGCTCGGGCTTCAATGACAGAGACAACCGGG | |
| | ACTGGGTGTCTGTAGTGACCCCAGCCCGGGATGGCCCCTGTGGGAC | |
| | AGTGTTGACGGTACGTCCTGGCCAGTGGGGGACAGAACCAGAGCAC | |
| | TGCCTGGTTCAAGCTTCAGCTCTATCACTTCCTAGTTATAGAAGCT | |
| | TTGGGGAGTTATTTAGCCTGGCTGTGCCTCAGTTTCATCAACTGTA | |
| | AAGTGGAGAAATAATAGTACCTACTCCACAGGTGTATTGAGAGGAT | |
| | TGAATGAGTTAATGTGTTGAAGTGATTAGGACAGTGACTGCACACA | |
| | GTAAGTGCTCAATAAACATCAGCTTCAAATAAAGAAAGCAATTCAT | |
| | GGTGATAGTTCTCCATTTTACAGATGGGAAAAGTAGGGCCATAGTA | |
| | GGGATTGTCTCAGCCAGTAGGCATCAGCATTGGGTTCAGAAACTAG | |
| | TCTATTTGTCTCCAGTGGTCCTGGAAGGCAAGGTCTGTATCCTTAA | |
| | TGTACAGAGAATCAGGACCTCACCTACAGAGGCCTCCTCAAATGAA | |
| | ACCCAGAGGTAAATGTTTGTGTGAGCAGAGCATTATCTCCTGCCAC | |
| | CTCCCTCATTACCAGAGGAAGAAAGAAAGAGAAAGAGAGGAAGAAA | |
| | GAAAGAGAAGGAAGGAAAGAAAGAAAGAAAGAGAAAAAGAGAGAGA | |
| | GGGAAGGAAGGAAGGAAGGAAGGAAGGAAGGAAGGGAGGGAGGGAG | |
| | GGAGGGAGGGAAGGAAGGAAGGAAGGAAGGAAAGAAAGGAAGAAAG | |
| | GAAGAAAGAAAGAAAGAGAAAGAAAGAAAGAAAGAAAGAAAGAAAA | |
| | AAAAAGAAAAGAAAGGAAGGAAGGAAGAAAGGAAGAAAGAAGGAAA | |
| | GAAGAAAGAAAGAAAGAAAGAAAAAGAAAGATTTAATGTTGGCCGG | |
| | GCATGGTGGCTCACGCCTGTAATCCCAGCACTTTGGGGGGCCGAGG | |
| | CGGGTGGATAACTTGAGGTCAGGAGTTTGAGACCAGCCTGGCCAAC | |
| | ACAGAGAAACCCCTTCTCTACTAAAAATACAAAAATTTGCTGGACC | |
| | TGTTGGTGGGCACCTGTAATCCCAGCTACTCGGGAGGCTGAGGCAG | |
| | GAGAATCACTTGAACCCAGGAAGCAGAGGTTGCAGTGAGCCGAGAT | |
| | TGCACCACTGCACTCCAGCCTGGGTGACAGAGTGAGACTCTGTCCC | |
| | AAAACAAACAAACAAAAAGAAATAATTCATGTCATTTTCCAAGGAT | |
| | GGCATTTGAAATTCCATTGATGGTGACTTGGCATATGGTTTTCAGT | |
| | TTTCAAGAGAGGAAGCACGGGGTGCTGAAAACAGTCTAGTCTTGTG | |
| | AGATGTCACATATTGTGGGCAATCCCTCAGTGACTCTGTTACTATG | |
| | AATCTCTGTCTTCTTGTCTGGGAAGCAGGGATGTGGCAGCGCCACT | |
| | TGTTAGGGTCACTGAAGGAACTAATGCTATGACTCAGGAGGGGTAT | |
| | TCCACTCGGGGTCTGGCACATGACATATGCGCATGAATGGTCATTG | |
| | CTGTTGTAACTTTTTTTTTCTAATTTATAAAGAAAAGATGATTAAT | |
| | TGGCTCGTTGTTCAGGCTGCACAGGGAGTTTTTAACTGTTGTGGGA | |
| | TTTTTTTTTTTTTTTTTGAGATGGAATCTCCCTGTCGCCGAGGCTA | |
| | AAGTGCAGTGGCACGATCTTGGCTCATTGCAAGCTCTGCCTCCCGG | |
| | GTTCAAGCGATTCTCCTGTCTCAGCCTCCCGAGTAGTTGGGATTAC | |
| | AGGCACCCACCACCACGCCCGGCTACTTTTTGTATTTTTAGTAGAG | |
| | ACAGGGTTTCACCATATTGGACAGGCTGGTCTCGAACTCCTGACCT | |
| | CAGGTGATCCGCCTGCCTCGGCCTCCCAAAGTGCTGGGATTACAAG | |
| | CGTGAGCTAATGCGCCCGGCCACAGGCAGCTTTTTATCAAGGAGAC | |
| | TAGGAGAGCGGCTCACAAGTAGCCAGACATGAGACCAGCAGATTTA | |
| | GCTTTGAAGTCCTACTCAGATTCTCATGCCCCTTTCTCTCATCCCC | |
| | ACCCCCCCTCCACCCCCATTCCCTGCAACAGAGGAATGAAACCCAT | |
| | GCCACTTACAGCAACACCCTCTACCTGGCAGATGAGATCATCATCC | |
| | GTGACCTCAACATCAAAATCAACTTTGCATGCTCCTACCCCCTGGA | |
| | CATGAAAGTCAGCCTGAAGACCGCCCTACAGCCAATGGTCAGGTGT | |
| | GGCCAGAGAGGGTCCCTAGGGCCCCTAGATGGTTCTAACCCCAAAC | |
| | CCCTTAACCATGAGCTTCCCTGTCAACTGCCACCCACAGGGAGCTG | |
| | GGAGTGAGGGCTGGGAATCAGGGTTGCCCAATGGAAGAGCCAGGAA | |
| | TTCTGGAGCCCAGGTTCAAATCTAGACTTTGTCATAAATGATGGTT | |
| | ATGCCCTGGCCAGTGGGGGACAGAGTCAAAGCACTGCCTGGTTCAA | |
| | GCCCCAGCTCTGTCACTTACTAGTTGTATGAGCTTGAGTGAGTTAT | |
| | TAAGCCTGGCTTTGGGAAATTGATAGTCCCTCTCTAGGTGTCAATA | |
| | TTTTCATCTGTGAAATGGGTTTAATAGTACTCTAAGAATTAAATGA | |
| | TATGGGAGGATCGCTTGAGCCCAGGAATTTGAGGCTGCTTAGAGCT | |
| | ATGATTGTGATGTGAATCAGATTCAACCATTAAACTGATGTAGATG | |
| | ATTCAGATGTGAATCAAATATTAAAGTCTCTACTTTTCAAAGAGGC | |
| | AGGGTGGTTGTGCCAGAGAGCAGGAATGGTCTCAGTCCAGACTGCT | |
| | ATGGAATGCTGGGCAAATCATATTTCATATTTGGACCTCAACTTCT | |
| | TTCCTTCTCTGTGAATGAAAGGAATTTGATTAAAGAGGATTTTAAA | |
| | AATTACGCTCCTTGCAAGTGTCTTGGTAGGGGCCAAGGGAGGTGCC | |
| | CTGGCAGAGTGCCTACAGGGGTAAACTATTCATACTGGGTGCCACG | |
| | TAGTATTTTATTTTATTTTTTTTAAAAGGAATCTGTTGCTAAATAA | |
| | AATTGAAAATCTTCAAATAGGATGAATCACAGGCCAATGATGGAAA | |
| | ATTATAGTTGTAATAAGAAATAAGATTTATTGATTATTTATTTATT | |
| | TATTTATTTATTTATTGAGACGGAGTCTCGTACTGTCGCCCAGGCT | |
| | GGAGTTCAATGGCATGATCTTGGCTCACTTCAACCTCCGCCTCCCA | |
| | GGTTCCAGTGATTCTCCTGCCTCAGCCTCCCAAGCAGCTAGGATTA | |
| | TAGGTGCCCACCACCACACCCAGCTAAGTTTTGTATTTTTAGTAGA | |
| | GACGGGGTTTCACCATGTTGGCCAGGCTGGTCTCAAACTCCTGACC | |
| | TCAGGCAATCCACCCACCTCGGCCTCCCAAAGTGCTGGGATTATAG | |
| | GTGTGAGCCACCGTGCCCTGCCAAGAACTAAGATTTATTGAGAGCT | |
| | TACTATATCTCAGGCATTATAGCCAGTGTTCTGCCTGGAACATCTC | |
| | ATCAAACTCTTACAGCAGCCCTAGGAGATAAAGGTATCATTATTAT | |
| | TACTATTCCCATTATACTGATGTGGAAATTGAGGCTCACAGAAGTT | |
| | TGAGTGACTTACCCAAAGCTGTAAAGTCTAGCAAGTGATAGAGCTG | |
| | GGATTCAGACATCAGGTCCAGAGCCTCCACTTGACCATGACCTTCC | |
| | CTTCGAAGCTTGCCCATGGTTTTCTGCTCTCTTGGCCAGCCAGACT | |
| | CAGGAAAGGATGCTGGGCACTGATGAGGGTGCACAGCTGACTTGGA | |
| | GGAAGAAGAGTGCTCATTGCAAGCTGTTGGCTGGAACCTCACCTGG | |
| | CCTAGCAGTGTCCTGGAGCAATGTGGCCCCGTCCTCGGCCTGGTAG | |
| | GCTCTGGGGAACAACAAGTGTGCCCACATCTCTCTGTTTTATGGTT | |
| | TTGTTTTGTTACGAATCCAGGTCTCACTCTGTCACCCAGGCTGGAG | |
| | TGCAGTGGCACAATCATAGCTCAATGCAGCCTCAAATTCCTGGTCT | |
| | CAAGCAATCCTCCCACCTCAGTCTCCCAGGTGGCTAGGACTACAGA | |
| | CACATGCCACCACACCACCCTGGGCTATTTTTAAAAATATTTTTAT | |
| | AGAGACAGGGTCTTGTTACATGGACCAGGCTAGTCTTAAACTCCTG | |
| | GCCTCAAATGATCCTCCTGCGTATGCCTCCTAAAGTATTGGGATTA | |
| | CAGGCGTGAGCCACAGCACCTGGTCCCTCTCTCTGTCTCGAGTTAG | |
| | ACATCTCACCCCAGTCTTCACTGATAACTCAGTTCTCATCTGTAAA | |
| | ATGGACTTCCACATTTCGGAGTATTTCTCATGTGCCTTATTTCATG | |
| | AGGCATCAGCCTTACAATGTCCTTTGTTGGATCATCCAGTGAAGTT | |
| | CACATCAGTGTGGCTGTTTTCAGTGTGGTTAAAAATAACTCATTTA | |
| | TAAACCTCAATCTTCTGATATAGATGTTAGTACATCACGATGGAGG | |
| | CCAGGACGCAAGTTGTAGGCCTCATCATAGCTCAATACCTTTTAAC | |
| | CTGTTCTAGATTGAGACCAAAGAAGGAATCCAGGTGTTCCTGTCAA | |
| | TAATTTGTTTTTTTGCCATCTTGAAAGCTACAGGGGTAGCAAAGCC | |
| | TATAGCAGTAGCAAGTGAGGGACTGGATGTGTTACTTGAGATGTGA | |
| | TTGATCCATAGACCAGCAGCATCAGCAGCATCCAGGCACTTGTCAG | |
| | AAATGCAGGACCTTGAGCCCCACCCCAATACTCACCAAATCAGAGC | |
| | CTGCATTTTATCTAGATCCCAAGTTGACCTGCGTGTACTTATTGCG | |
| | GTTTGCAAAGCAACAGTTGGTGGGTTCCACTCTTATTGCTGGATAA | |
| | AAATGCAAATAACTGCAAGGGACTGCCTAGGAATGCAAATCAGAGA | |
| | AGGTGGCCTATCTGCAGATGTTCTCAGCCCGGTCCTCTCACCAACC | |
| | CTTCTCCCCTGGCAGTGCTCTAAACATCAGAGTGGGCGGGACCGGC | |
| | ATGTTCACCGTGCGGATGGCGCTCTTCCAGACCCCTTCCTACACGC | |
| | AGCCCTACCAAGGCTCCTCCGTGACACTGTCCACTGAGGCTTTTCT | |
| | CTACGTGGGCACCATGTTGGATGGGGGCGACCTGTCCCGATTTGCA | |
| | CTGCTCATGACCAACTGCTATGCCACACCCAGTAGCAATGCCACGG | |
| | ACCCCCTGAAGTACTTCATCATCCAGGACAGGTAAGGCAAAGGTTC | |
| | CTACATGGGAACTCATGGGTAGAATTCAGAGGGGGTCTTTGGACTT | |
| | GGATGGAGGAAAATTGCATCTTTTTTTTTTTTTTTTTTGACAGAGT | |
| | CTTGCTCTGTCGCCAAGGTTGGAGTGCAATGGTGTGACCTCGGCTC | |
| | ACTGCAACCTCCGCCTCCTGGGTTCAAGTGATTCTTCTGCCTCAGC | |
| | CTCCCAAGTAGCTGGGATTACAGGCAGAGGCCAGCAGGCCTGGCTA | |
| | ATTTTTGTATTTTTAGTAGAGATGGGGTTTCACCATGTTGACCAGG | |
| | CTGGTCTTGAACTCCTGACCTCAAGTGATCCACCCCCTTGGCCTCC | |
| | CAAAGTGCTGGGATTACAGGTGTGAGCCACCACGCCTGGCTGAAAA | |
| | ATTGCATCTTTATATTCATTACCCTTTAGCTGAACCTTAGCATTCC | |
| | CATCCATTATAAATACAGGCAACAAACTACAGAAATATTAGCAGGT | |
| | CGTGTGAAGCCATTTATAGTCATCAGTACTTTTTAGTTACTATAGC | |
| | ATTAGAATTTTCACTAGACCTTATTATTTAATGAATTATCAATACA | |
| | TATGTATTGGCAAATATATATTTATGTATTATATATATATATATAT | |
| | ATATACACACACACACATATATATACACACACACACACAAAGATAT | |
| | AATGCATTTTGTATTTCAAATGTCTTTTTTTTTGGTCGTTTGATTT | |
| | GGTTTGGTTTTTTTGAGACAGGGTCTCATTCTGTCACCCAGGCCGG | |
| | AATACAGTGACTTGATCATAGCTCACGGCAGCCTCAACCACCCAGG | |
| | CTCAAGCGATCCTCCAGCTTTAGCCTCATGAGTAGCTGGGACTACA | |
| | GGCACACACCACCATACACAGCTAGCAAATGTTTTTAATATTTTGA | |
| | GAATGGTGCTTCAATTTAATTGATTTCCTTTATAATCTTTTAAATA | |
| | TTTTATTTTAGGCATTTAAAAGTATTATTTCTGAGGAGGGATGTAC | |
| | AAGCTTCACCAGAGAGCTGAGTAGGGTTTATAATTCAAAAAATGGT | |
| | AAGAAACTTGATGGAAAGGCTTGCCCAGGGCAGCAGGCAGCCCAGC | |
| | ACCTGGGGGAGGAGTAAGACATGGGCATGGTTAGCCAAGGGCCAAT | |
| | TGAAACTAGGACACAAGTGTGCTACCTTTTACTCAAGAGGCACTAG | |
| | ATGACTATGAACCTTACCATGAGACGGTGATTCACTTTCTCGTTCA | |
| | TAAGTTGAAAAGATATTTTGGAAGCTTGCTGTGTGTCGGGCTCTGT | |
| | GGATTTCACAGTAAATAGAAGAAACAAAAACCTCTGCCCTCACACA | |
| | GCTTATATTCTAAAGGGGGAGGGCCAACTATTAAATGAAAGTGAAA | |
| | TATATAGTATGTTAATGACAAAGAGGTTGTAGAAGAAACAAAGCAG | |
| | GAGAAGAAAGACATTTGCAATTTTAATTGCATTGTCAGAAAAAGCA | |
| | TTCCTGAGAGACAGTGGTCTGTTTCTGCCTGGTTACACCAAGGGAA | |
| | TATTTTCAGGTGAGTGCCAATGTGATGTTCACACGTCTTTGGAATC | |
| | TCCCTTTGCAATAAAAATAAAGCAGAAGTCTGTTTCAGAGCATTTT | |
| | GCAATTAGCAGAGTCTAACTCAAACTTAATACAACTCTTTTGTTGA | |
| | TCTTGTCCTCATTGTATGGTCCTGTTCTATTTATGCAAGTGATACT | |
| | TGTCTGCCATTTGAATTCATGACCTAAAGATTCTTCTAGTAAGTAA | |
| | ATGTATTTCAGTAAAAAATACAAGTGGATTTAAAGAAAAATAGTAA | |
| | GTAAATAATAATACAGATGTTGGGACACACAGGAAAACGCTCATAA | |
| | AGGTGGTATCTCAATAAAGAAAAAAAGAACTGAAAGTTGGAAACTG | |
| | TTGGCATGACCTTGATTGGAGTATCACAGAGATTTGGGTTCAAATC | |
| | TTGGCTTTGCCATTTCTTAGCTTTAAGAAACAGGTTAGCTCATCTC | |
| | TCTGGGTTTTTCTATTCTCAGACGTAAATGGGGATAAATAATACCT | |
| | GCTATGCTGGGATTTTAAAACTTGTTTTTCATGAGTATAACGGGCA | |
| | CGATGGCTCACGCCTGTAATCCCAGCACTTTGGGAGGCTGAGGCAG | |
| | GCAGATCACCTGAGGTCAGGAGGTCAAGACTGGCCTGGCCAATACA | |
| | GCAAAGCCCATCTCTACTAAAAATACAAAAAAATTAGACAGGCATG | |
| | GTGGTGGGCGCCTGTCATCCCAGCTACTTGGGAGGCTGAGGCAGGA | |
| | GAATTGCTTGAACCCAGGAGGCAGAGGTTGCAGTGAGCCAAGATCA | |
| | CACCATTGCACTCCAGCTTGGGCAACAAGAGTGAAACTCTACCTCA | |
| | AGAAAATAAAAATAAAGCATAGCAGGGAGGCCGAGGCAGGTGGATC | |
| | ACGAGGTCAGGAGTTCGAGACCAGCCTGGTCAACATGGTGAAACCC | |
| | TGTCTGTACTAAAAATACAAGAATTAGCCAGGCATGATGGCACACA | |
| | CCTGTAATCCTAGCTACTCAAGAGGCTGAGACAGGAGAATTGCTTG | |
| | AACTGGGAGGCAGAGGTTGCAGTGAGCCAAGATCACACCACTGCAC | |
| | TCCAGCCTGGGTGGCAGAGCAAGACCCCATCTAAAAATAAAATAAA | |
| | ATAAAAGTACAGCAGGGACATTGCGGAAAACTTGGAAAGTTTTGAA | |
| | AAAGAAGCAGGAAAAAATGTAGGGTGTGTCTATAAGTTGCCATCTG | |
| | GGTGAGTGTGGGAGCTGGGGTGAGAACTACCAGTGTAGGATAGGTG | |
| | TCAGAACATCACAAGGCATGAAAAGAGGAGACAAGTCGGGGAGACA | |
| | GTGAAAGTGACTGGAGGGGACACATGGATGTCCAGGTTGGCTTGAG | |
| | CACGTGGAGCCCTCACAGGCCATACTGTCTGCCTTTCAGGAGTTGA | |
| | GTCCACTTAACCTCAGGGCTTTTCTCTTGCCAGGCAGAGTCTGCAC | |
| | TTTTGGAGCCAGATCTGAATTATGGGAGACACAGAGCTAAGAGTGA | |
| | AAAACACTCCCAGAAGCTCCCGTCTCAGTTTTGCAGTCAGTGTTCA | |
| | GCCTCCCTCCGAAATCAACTTTGAGGAAAGAGTGCGGAATGGCAGA | |
| | GGGGGTGCCCAGTTTGCCTCCCCAGAAAGCCTATGGGTATCCTGAC | |
| | CCAGCCCAGCACATGTGGGAGTCTCTGCATGCTCTATTTCGGGTTT | |
| | CTCCTTCTAACTGTGTTTGGGTGCAATTGTGTATACGTGCAGATGG | |
| | GTGCACACACTCCAATTTCATCAGTGGCTCTCGGTACCCAGAGGTT | |
| | TCCATTGTTATAATTATACCAGGCATATTGTAGATAGCACATAGCA | |
| | GCTAATAATTTTTGAATGTCATTGCTGGGAAATCAGGAAGTGCTGA | |
| | CTTTTGGATAGTTTCAGCTCTGCACTGATGACAGTTTCACTTTAGT | |
| | ATCAAATATATAAAGCACCTATGGCATGCTACACACAGTTCTACGC | |
| | ACTTTGAGAAATTAACGAATTTAATCCTTACAGCTATCTTATCACA | |
| | TGGGCTGCGATCATTTCTTATGTACAGATGGAGAAATTGAGGTACA | |
| | CAGAGATTAAATAAGTTGCCCAAAGTCACACAGCTGAAAGGTGACA | |
| | GAGCAATATTTGAATCCAGGAAGTCTGACTCCAGAATCTTTATTTA | |
| | ACCAACCACATTAAATGATGTGTAACCCTCCCAGATGCCCACACAC | |
| | TAGAGACTCAACTATCCAAGTGGTGGAGAATGGGGAGTCCTCCCAG | |
| | GGCCGATTTTCCGTCCAGATGTTCCGGTTTGCTGGAAACTATGACC | |
| | TAGTCTACCTGCACTGTGAAGTCTATCTCTGTGACACCATGAATGA | |
| | AAAGTGCAAGCCTGTGAGTTGACTCCCCTCCCCCAGCCCATCTCTT | |
| | GTAACCAAAGACATTTGGCCACAAAGAAAACAAATCAATATTTCTT | |
| | CCCTGTTTCCCTCTTTTACCAGAGGGATAGAATGAGCAATAAGATG | |
| | AGGTGGGCGTGGCTAGGCAGGAAACCTAAGCTGCAGGGGAAATCAG | |
| | GTGGGATCAGTAAAGTGCGGCAGGCTGGTAAGAGCTCTGGCTTATC | |
| | TGCAAGCTTGTGTTCAAATAACAGGAGCTGACATTTTTAAGCACTG | |
| | TGCCCACTTTATTTGTGTTAGCTTTTAATGCTTCACAGCAACTCTA | |
| | TAAGGGAGGTATTACTGGTTCCCTTTCCTCATGAGGAGAGGGGCTC | |
| | AGAGAACTTCAGTGGCTTGCCTGAGATCATGCATCTATCTAACAAA | |
| | TGGCAGAGCTGGGACCTGCACGAGCCCCGGTATACAGGTCTCCTAA | |
| | CAACTTCTGCCTGGGGCAAGGGAAGGCACCTGTGAGGTGGGCAGTC | |
| | CACTCCACGTGGCAGAACCACATTCAGGCTCCTTCATGGAGGGTGT | |
| | TTTTCTATTGCCCTCTCCCTGTAGACCTGCTCTGGGACCAGATTCC | |
| | GAAGTGGGAGTGTCATAGATCAATCCCGTGTCCTGAACTTGGGTCC | |
| | CATCACACGGAAAGGTAAGAGAGCCACTCGCTCCTCAACATTCCTG | |
| | GCTGGGAAAGATTTCTGGAGAGGAAGAGGGATAACAGAGCCTGGCA | |
| | CCTTGGCACCTTACTGAGCTCTGAAGAACTGGGAGCAAGTGGATCC | |
| | TCTGGGGCAAGGTGGAATACAGACTGCCTTCCTTTCACTATTCCCA | |
| | TTCATACACCCATTCATTGGACAAATATGATTTGTAGATGAATGTA | |
| | ACACAGGACACGGGGGAGGCCAGACAGGCAGGTAGGAATGTAGATC | |
| | ATCCAGACAAGTTTGCACACACATGAGGCCACAAACATACATTCAT | |
| | ATTTGTAGTAGATGTGTGCCTACTCTGTGTCTGGAGGTCTTGTGAT | |
| | GAGCAGAAGTAGATGGCCCTGCTCCCATGGAGCTTGTGAATCAGTG | |
| | AAGAAGGTGCCATTTGAATAATCCCCTGTAGATGTGTAGGATCTGG | |
| | GTGATCCATTAAGTGCAGGGTCTACAAAATATCTCAAGCACTGATC | |
| | TTAGGAGTGGTTTAGGAAGGGTCAGAATCTTGTGGCTTCCAGCTGC | |
| | ATGACTCCTAAACCTTAATTTCTAATCTTGTAGCTAATTTCTTAGT | |
| | TCTACAAAGGCAGTCTAGCCCCCAAGGCAAGAAGGAGGTTTGTTTT | |
| | TGGAAAGGGCTGTTATCGTCTTTGTTTTAAACTATAAACTAAGTTC | |
| | CTTCCAAATTAGTTCAGCCTACACCCAGGAAGGAACAAGCACAGCT | |
| | TAAAAGTTAGAACCAAGATGGGGTTGGCTAGGTTGGCTCTCTTTCA | |
| | CTGTCTCAGTCATAATTTTGCAAAGGTCATTTCAAGAGTACAGCTT | |
| | GGGCAAAGGGCCTGTGGCAGGAGGGAGCCTGGCCTGTTAAAGGAAC | |
| | CCAGAGAAGGCCAGGGGAGTTGTGTGGGATGAGCTGCTGCAGACCT | |
| | TGTGGAGCCTTGCAGTTCTTGCTAAGGGTTGGGACCTTTCTCCTGA | |
| | AAGCAGTGAGAGCCCCTGATGGGTCTGAAGTAGGGGAGTAACATGA | |
| | TCAGATTTGGGTTTTGAAGAGATCAGTCTGGCTGCGAAGTGAAAAG | |
| | TAGATTGAAGGGGTTTCTGTCAGGATGCTTGCCAAATCTCATGCAT | |
| | TCTTTATTCACTGATCCTTCTGTTTTCCTCCAAAGGTGTCCAGGCC | |
| | ACAGTCTCAAGGGCTTTTAGCAGCTTGGGTAAGTTCAGGTCCTTTC | |
| | TGCAGTGGGACCTGTTCCAGAACTCTCCTGGGGGGCTTCTATCTGT | |
| | TAACTTGTAATGCTTCATAGCAACTCTATAAGGGAGGTGTTACTAG | |
| | TTCCGTTTTCTAAATAAGGAGAGTGGCTCAGAGAACCTGAGTGGTC | |
| | CTACTGAGATCATGCATCTCTCTATTAATTGTAAGAGCTGGGACAT | |
| | GTGCCCCAGTATACAGATCACTTATTTTATACACAAACTTATTACA | |
| | AACCCAAAGGATATGCACACACTCCAGATCTGCATCTTGGGCACTT | |
| | ACTGAAGCTCCAAGCCTCCTCTCTAACACTTTTTTCTAGATTCATT | |
| | CAATAGGTGACAAATAGGCCAGGCGCAGTGGCTCACGCCTATAATC | |
| | CCAGCACTTGGGGAGGCTGAGGCGGGCGGATCACAAGGTCAAGAGA | |
| | TTGAGACCATCGTGGCCAACATGGTGAAACCCCATCTCTACTAAAA | |
| | ATACAAAAGTTAGCTGGGTGTGGTGGCGCGCGCCTCCCAGCTACTT | |
| | GGGAGGCTGAGGCAGGAGAATGGCTTGAACCACGGGGGGCAGAGGT | |
| | TGCAGTGAGCCAAGATCACGCCACTGCACTCCAGCCTGGCAACAAA | |
| | GCAAGATCCATCTCAAAAAAAAAAAAAAAATAGGTGACAAATATTC | |
| | CTTGAGTGCTACCATGTGCCAAGAGCCATGTTTAGTTCTGGGGTAA | |
| | ACACAACAAATAAGAAACCCCACTTCTCTGCTTCTTTGCTCCTCTA | |
| | ACTCCCTGAGGTCCCAGGGTGTGCTGGAGGGCTGGGCTTGAGCTCT | |
| | GAAGAGGCAACTGTATCTATGGAAATGCCTGGAGTGCCTGGACTTA | |
| | GGGCTGTACACATGCACACATACCTATGCTGTGTCCCAATAGTACA | |
| | TATGTGCACACAGACACAGGCATGCCCAGATAATCACATGAGAATG | |
| | TAGGTCATCCAGACATGTTTACCCACACACACAAGGTCACGTACAT | |
| | GCATTTATATTTGCATGTACAGATGCACAGAAAGCTGCAATTGTCT | |
| | GCATGCATATTTGTGTGTGCAGATGTACACACACTTATTGGAAACC | |
| | TGTACCCAAAGGATATGAACATACCCCAGATCTGTATATTGGGCAC | |
| | TTCCTGAAGCTCCAAGGCTCCTCTCTAACACCTTTTTCTAGATTCA | |
| | TTCAATAGGTGATGAATATTCGTTGAGTAATACTATATGCCAAGAA | |
| | CCATGCTTAGTGCTGGGGCAAACACAACGGCCAAACAAGAACCCCC | |
| | ACTTCTCTGCTTCTTTGCTTTTTCTGACCACAAAGGCCAGGGACAG | |
| | GTGGCATTTTTATGGCCCATGGTTTTCATGGCACTGAATTGTCTAA | |
| | AAATCTAGGTGAAAAGCAAATCTATGCAACAGCTACTGGTACCTGG | |
| | ATCCAAGGAGGTTCTTACACTAACTTGGCTTTTTTTTTTTTTTTTT | |
| | TTTTTGAGATGGAATCTGCTCTGTCACCCAGGCTGGAGTGCAGTGG | |
| | TGAAATCTCAGCTCACTGTAACCTCTGCCTCCTGGGTTCAAGCGAT | |
| | TCTTCTGCCTCAGCCTGCCCAGTAGCTGGAACTACAGGCGTGCACC | |
| | ACCACGCCCGGCTAATTTTTGTATTTTCCAGTAGAGATGGGGTTTC | |
| | TCCGTGTGGCCAGGCTGGTCTTGAACTCTTGACCTCAGGAGATCTG | |
| | CCTGCCTTGGCCTCCCAAAATTCTGGGGTTAAAGATGTGAGCCACC | |
| | CCACCCGGCTAACCGGGCATTTGACCTGAACTAGTTCAGCCCAGTG | |
| | CTGGTGAGCTCAGAGATGGAGAGTCACAGCACTCCTAAGAGGCTTG | |
| | CTCTCTGTGACTATCCCCCAAGGTCTCTAAGGATGGTTGGATAGAT | |
| | TGGGCACTTCACAAGAATGCCCTTTGCCCTTTTGAGGAGGTACCAA | |
| | GCCTAGTGCCGGAGGAAAGATTATCTTTTTCAAATCGGTCCACCTT | |
| | TTTCAGGGCAGATAAGGAGGAAGCTTCCTTTTTCTAGGAGAGCAGC | |
| | CCAGAGAGGGTGTCCTCTTCTGATTGGTCAGCCTAGACGAGGCAGC | |
| | TTATGTTAATTTGCACAAAAGTACAGCAGTACTAGCAGTTGCCCTG | |
| | TCACTGTTTTCTTTTCAGGGCTCCTGAAAGTCTGGCTGCCTCTGCT | |
| | TCTCTCGGCCACCTTGACCCTGACTTTTCAGTGACTGACAGCGGAA | |
| | AGCCCTGTGCTCCATGGCTGCCATCTCACCTCCTGCTGGGCAGGGG | |
| | GCATGATGCGGGCCAGTGCTCCAGCCACAGAAAAGAAAGTTCATGC | |
| | TTTGTTCAGCCTGCCTTCTTTTCTCCCTTTTAATCCTGGCTGTCGA | |
| | GAAACAGCCTGTGTCTTTAAATGCTGCTTTTTCTCAAAATGGGACT | |
| | TGTGACGGTGTACCTGAGGCCCCCATCTCCTTAAAGAGTGTGGCAA | |
| | AATAATGATTTTTAAATCTCA | |
| Exon 2 with | ATCCATCTCTGTTCACAGGACACCAGACATCAGAGACAGAGAGAAA | 2 |
| junctions | AATTCAAAGGGCCAACCCGTCTTTCCTTTGGGCAGGTGCTATCTAG | |
| | ACCTGAAGTAGCGGGAAGAGCAGAAAGGATGGGGCAGCCATCTCTG | |
| | ACTTGGATGCTGATGGTGGTGGTGGCCTCTTGGTTCATCACAACTG | |
| | CAGCCACTGACACCTCAGAAGCAAGTAAGTGAAA | |
| Exon 5 with | CCTAACCTGTTTCAGATATCTCCCTCCTGGAGCACAGGCTGGAATG | 3 |
| junctions | TGGGGCCAATGACATGAAGGTGTCGCTGGGCAAGTGCCAGCTGAAG | |
| | AGTCTGGGCTTCGACAAGGTCTTCATGTACCTGAGTGACAGCCGGT | |
| | GCTCGGGCTTCAATGACAGAGACAACCGGGACTGGGTGTCTGTAGT | |
| | GACCCCAGCCCGGGATGGCCCCTGTGGGACAGTGTTGACGGTACGT | |
| | CCTG | |
| Exon 6 with | ACCCCCATTCCCTGCAACAGAGGAATGAAACCCATGCCACTTACAG | 4 |
| junctions | CAACACCCTCTACCTGGCAGATGAGATCATCATCCGTGACCTCAAC | |
| | ATCAAAATCAACTTTGCATGCTCCTACCCCCTGGACATGAAAGTCA | |
| | GCCTGAAGACCGCCCTACAGCCAATGGTCAGGTGTGG | |
| Exon 8 with | AGATGCCCACACACTAGAGACTCAACTATCCAAGTGGTGGAGAATG | 5 |
| junctions | GGGAGTCCTCCCAGGGCCGATTTTCCGTCCAGATGTTCCGGTTTGC | |
| | TGGAAACTATGACCTAGTCTACCTGCACTGTGAAGTCTATCTCTGT | |
| | GACACCATGAATGAAAAGTGCAAGCCTGTGAGTTGACTC | |
| Exon 9 with | TGTAGACCTGCTCTGGGACCAGATTCCGAAGTGGGAGTGTCATAGA | 6 |
| junctions | TCAATCCCGTGTCCTGAACTTGGGTCCCATCACACGGAAAGGTAAG | |
| | AGAGCCACTCGCT | |

In certain embodiments, the degree of complementarity between the target sequence and antisense targeting sequence is sufficient to form a stable duplex. The region of complementarity of the antisense oligomers, or pharmaceutically acceptable salts thereof, excluding the abasic units, with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligomer, or a pharmaceutically acceptable salt thereof, of about 14-15 bases is generally long enough to have a unique complementary sequence. In certain embodiments, a minimum length of complementary bases may be required to achieve the requisite binding Tₘ, as discussed herein.

In certain embodiments, oligomers as long as 40 bases may be suitable, where at least a minimum number of bases, e.g., 10-12 bases, are complementary to the target sequence. In some embodiments, facilitated or active uptake in cells is optimized at oligomer lengths of less than about 30 bases. For PMO oligomers, described further herein, an optimum balance of binding stability and uptake generally occurs at lengths of 18-25 bases. Included in the disclosure are antisense oligomers, or pharmaceutically acceptable salts thereof, (e.g., PMOs, PMO-X, PNAs, LNAs, 2′-oMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases, in which at least about 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous or non-contiguous bases that are complementary to the desired target sequences.

In certain embodiments, antisense oligomers, or pharmaceutically acceptable salts thereof, may be 100% complementary to the target sequence (excluding at least one abasic nucleotide), or may include mismatches, e.g., to accommodate variants, as long as a heteroduplex formed between the oligomer and target sequence is sufficiently stable to withstand the action of cellular nucleases and other modes of degradation which may occur in vivo. Hence, certain oligomers may have substantial complementarity, meaning, about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligomer (excluding at least one abasic nucleotide) and the target sequence. Oligomer backbones that are less susceptible to cleavage by nucleases are discussed herein. Mismatches, if present, are typically less destabilizing toward the end regions of the hybrid duplex than in the middle. The number of mismatches allowed will depend on the length of the oligomer, the percentage of G: C base pairs in the duplex, and the position of the mismatch(es) in the duplex, according to well-understood principles of duplex stability. Although such an antisense oligomer, or a pharmaceutically acceptable salt thereof, is not necessarily 100% complementary to the target sequence, it is effective to stably and specifically bind to the target sequence, such that splicing of the target pre-RNA is modulated. The stability of the duplex formed between an oligomer and a target sequence is a function of the binding Tₘ and the susceptibility of the duplex to cellular enzymatic cleavage. The Tₘ of an oligomer with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligomer Hybridization Techniques, Methods Enzymol. Vol. 154 pp. 94-107. In certain embodiments, antisense oligomers, or pharmaceutically acceptable salts thereof, may have a binding Tₘ, with respect to a complementary-sequence RNA, of greater than body temperature and greater than about 45° C. or 50° C. Tₘs in the range 60-80° C. or greater are also included. According to well-known principles, the Tₘ of an oligomer, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. In certain embodiments, the compounds show high Tₘ (45-50° C. or greater) at a length of 25 bases or less.

In certain embodiments, antisense targeting sequences are designed to hybridize to a region of one or more of the target sequences listed in Table 2. Selected antisense targeting sequences can be made shorter, e.g., about 12 bases, or longer, e.g., about 40 bases, and include a small number of mismatches, as long as the sequence is sufficiently complementary to effect splice modulation upon hybridization to the target sequence, and optionally forms with the RNA a heteroduplex having a Tₘ of 45° C. or greater.

### V. Cell-Penetrating Peptides (CPPs)

In an aspect, the antisense oligomer, or a pharmaceutically acceptable salt thereof, described herein further comprises (e.g., is conjugated or covalently attached to) or associated with (e.g., forms a complex with) a delivery agent. Any delivery agent that increases cellular uptake of the antisense oligomer, or of a pharmaceutically acceptable salt thereof, is contemplated for use herein. For example, the antisense oligomer, or a pharmaceutically acceptable salt thereof, may further comprise (e.g., be covalently attached to the delivery agent) such as a cell-penetrating peptide, an antibody, a fragment of an antibody, an antigen fragment of an antibody, at least one ligand, or a combination thereof. In an embodiment, the antisense oligomer, or a pharmaceutically acceptable salt thereof, is conjugated or complexed to an antibody or antibody fragment. Antibodies and antibody fragments that affect cellular uptake are known in the art, and any such antibody or antibody fragment may be associated or form a complex with the oligomer (e.g., attached by covalent bond), or a pharmaceutically acceptable salt thereof, of the present disclosure. In some embodiments, the antisense oligomer, or a pharmaceutically acceptable salt thereof, is conjugated to an antibody is an anti-TfRI antibody. Examples of such anti-TfRI antibodies, means of conjugation to an antisense oligomer, or to a pharmaceutically acceptable salt thereof, complexes comprising antisense oligomers, or a pharmaceutically acceptable salt thereof, covalently linked to an anti-transferrin receptor 1 (TfRI) antibody, and formulations thereof for use in the instant disclosure can be found in WO/2023/283623 and WO/2023/283624, both of which are incorporated herein by reference in their entireties.

In some embodiments, the antisense oligomer, or a pharmaceutically acceptable salt thereof, is an antibody-peptide-oligomer conjugate or complex, for example, of formula A-(Xi-B—X₂-D) n or A-(Xi-D-X₂—B) n, wherein A is an antibody or antigen binding fragment thereof; B is a polynucleotide; D is an endosomolytic peptide or a membrane penetrating peptide; Xi is a bond or a first non-polymeric linker; X₂ is an optional bond or an optional second linker; and n is an integer >1, as described in WO/2022/212886, which is incorporated herein by reference in its entirety.

In another embodiment, the antisense oligomer, or a pharmaceutically acceptable salt thereof, is conjugated to a cell-penetrating peptide. The antisense oligomer, or a pharmaceutically acceptable salt thereof, of the present disclosure may be covalently linked to a cell-penetrating peptide as described herein or as known in the art.

In particular, arginine-rich cell-penetrating peptides (CPP) discussed herein, e.g., within the scope of substituent J, can be effective in enhancing penetration of antisense oligomers, or pharmaceutically acceptable salts thereof, into a cell and to cause exon skipping in different muscle groups in animal models. Exemplary arginine-rich peptides are provided in Table 3.

TABLE 3
Arginine-Rich Peptide Transporters (J).

| | | SEQ ID |
|---|---|---|
| Name (Designation) | Sequence | NO. |
| rTAT | RRRQRRKKR-aa | 179 |
| Tat | RKKRRQRRR-aa | 180 |
| R₉F₂ | RRRRRRRRRFF-aa | 181 |
| R₅F₂R₄ | RRRRRFFRRRR-aa | 182 |
| R₄ | RRRR-aa | 183 |
| R₅ | RRRRR-aa | 184 |
| R₆ | RRRRRR-aa | 185 |
| R₇ | RRRRRRR-aa | 136 |
| R₈ | RRRRRRRR-aa | 137 |
| R₉ | RRRRRRRRR-aa | 138 |
| (RXR)₄ | RXRRXRRXRRXR-aa | 139 |
| (RXR)₅ | RXRRXRRXRRXRRXR-aa | 140 |
| (RXRRBR)₂ | RXRRBRRXRRBR-aa | 141 |
| (RAR)₄F₂ | RARRARRARRARFFC-aa | 142 |
| (RGR)₄F₂ | RGRRGRRGRRGRFFC-aa | 143 |
| D-Pep 1.9b | RBRBYLIQFRBRRBR-aa | 144 |

Sequences assigned to SEQ ID NOs do not include the linkage portion “-aa”. In the amino acid sequence, A represents Alanine, B represents β-Alanine (also represented as β-Ala or β), F represents Phenylalanine, G represents Glycine, L represents Leucine, R represents Arginine, X represents 6-Aminohexanoic acid (also represented as Ahx or «), C represents Cysteine, I represents Isoleucine, K represents Lysine, Q represents Glutamine, and Y represents Tyrosine. In some embodiments, an antisense oligomer, or a pharmaceutically acceptable salt thereof, disclosed herein is a conjugate of D-Pep 1.9b wherein-aa is isoglutamine (IsoGln). In some embodiments, an antisense oligomer, or a pharmaceutically acceptable salt thereof, disclosed herein is a conjugate of D-Pep 1.9b wherein-aa is isoglutamine (IsoGln). Examples of such conjugates can be found in WO/2022/171972, which is incorporated herein by reference in its entirety.

In another aspect, exemplary cell-penetrating peptides within the scope of substituent J are provided in Table 4. The point of connection to substituent “-aa” is as shown in the Table.

TABLE 4

| Exemplary Carrier Peptide Sequences | | |
|---|---|---|
| | | SEQ ID |
| Name | Sequence (Amino to Carboxy Terminus) | NO |
| (RFF)₃; CP0407 | RFFRFFRFF-aa | 186 |
| RTR | RTRTRFLRRT-aa | 187 |
| RFFR | RFFRFFRFFR-aa | 188 |
| KTR | KTRTKFLKKT-aa | 189 |
| KFF | KFFKFFKFF-aa | 190 |
| KFFK | KFFKFFKFFK-aa | 191 |
| (RFF)₂ | RFFRFF-aa | 192 |
| (RFF)₂R | RFFRFFR-aa | 193 |
| RX | RXXRXXR-aa | 194 |
| (RXR)₄; P007 | RXRRXRRXRRXR-aa | 139 |
| | RBRBYLIQFRBRRBR-aa | 144 |
| Tat₄₇_58 | YGRKKRRQRRR-aa | 195 |
| Tat₄₈_58 | GRKKRRQRRR-aa | 196 |
| Tat₄₉_58 | RKKRRQRRR-aa | 180 |
| Penetratin | RQIKIWFQNRRMKWKKGG-aa | 197 |
| Transportan | GWTLNSAGYLLGKINLKALAALAKKIL-aa | 198 |
| 2XHph-1 | YARVRRRGPRGYARVRRRGPRR-aa | 199 |
| Hph-1 | YARVRRRGPRR-aa | 200 |
| Sim-2 | AKAARQAAR-aa | 201 |
| HSVI VP22 | DAATATRGRSAASRPTERPRAPARSASRPRRPVE-aa | 202 |
| Pep-1 | KETWWETWWTEWSQPKKKRKV-aa | 203 |
| Pep-2 | KETWFETWFTEWSQPKKKRKV-aa | 204 |
| ANTP | RQIKIWFQNRRMKWKK-aa | 205 |
| R₆Pen | RRRRRR-RQIKIWFQNRRMKWKKGG-aa | 206 |
| rTAT | RRRQRRKKRC-aa | 207 |
| pTat | CYGRKKRRQRRR-aa | 208 |
| R₉F₂ | RRRRRRRRRFFC-aa | 209 |
| R₉CF₂ | RRRRRRRRRCFF-aa | 210 |
| R₈CF₂R | RRRRRRRRCFFR-aa | 211 |
| R₆CF₂R₃ | RRRRRRCFFRRR-aa | 212 |
| R₅FCFR₄ | RRRRRFCFRRRR-aa | 213 |
| R₅F₂R₄ | RRRRRFFRRRR-aa | 182 |
| R₄CF₂R₅ | RRRRCFFRRRRR-aa | 214 |
| R₂CF₂R₇ | RRCFFRRRRRRR-aa | 215 |
| CF₂R₉ | CFFRRRRRRRRR-aa | 216 |
| CR₉F₂ | CRRRRRRRRRFF-aa | 217 |
| F₂R₉ | FFRRRRRRRRR-aa | 218 |
| R₅F₂CF₂R₄ | RRRRRFFCFFRRRR-aa | 219 |
| R₉I₂ | RRRRRRRRRII-aa | 220 |
| R₈F₃ | RRRRRRRRFFF-aa | 221 |
| R₉F₄ | RRRRRRRRRFFFF-aa | 222 |
| R₈F₂ | RRRRRRRRFF-aa | 223 |
| R₆F₂ | RRRRRRFF-aa | 224 |
| R₅F₂ | RRRRRFF-aa | 225 |
| (RRX)₃RR | RRXRRXRRXRR-aa | 226 |
| (XRR)₄ | XRRXRRXRRXRR-aa | 227 |
| (RX)₅RR | RXRXRXRXRXR-aa | 228 |
| (RXR)₃ | RXRRXRRXR-aa | 229 |
| (RXR)₂R | RXRRXRR-aa | 230 |
| (RXR)₂ | RXRRXR-aa | 231 |
| (RKX)₃RK | RKXRKXRKXRK-aa | 232 |
| (RHX)₃RH | RHXRHXRHXRH-aa | 233 |
| (RX)₇R | RXRXRXRXRXRXR-aa | 234 |
| (RXR)₅ | RXRRXRRXRRXRRXR-aa | 140 |
| (RXRRBR)₂; B | RXRRBRRXRRBR-aa | 141 |
| (RXR)₃RBR | RXRRXRRXRRBR-aa | 235 |
| (RB)₅RXRBR | RBRBRBRBRBRXRBR-aa | 236 |
| RBRBRBRXRBRBRBR | RBRBRBRXRBRBRBR-aa | 237 |
| X(RB)₃RX(RB)₃R—X | XRBRBRBRXRBRBRBRX-aa | 238 |
| (RBRX)₄ | RBRXRBRXRBRXRBRX-aa | 239 |
| (RB)₄(RX)₃R | RBRBRBRBRXRXRXR-aa | 240 |
| RX(RB)₂RX(RB)₃R | RXRBRBRXRBRBRBR-aa | 241 |
| (RB)₇R | RBRBRBRBRBRBRBR-aa | 242 |
| R₄ | RRRR-aa | 183 |
| R₅ | RRRRR-aa | 184 |
| R₆ | RRRRRR-aa | 185 |
| R₇ | RRRRRRR-aa | 136 |
| R₈ | RRRRRRRR-aa | 137 |
| R₅GR₄ | RRRRRGRRRR-aa | 244 |
| rTAT | RRRQRRKKR-aa | 179 |
| | RBRRBR-aa | 245 |
| | RXRRBR-aa | 246 |
| | RBRRXR-aa | 247 |
| | RXRYBRXR-aa | 248 |
| | RBRYBRBR-aa | 249 |
| | RXRYBRBR-aa | 250 |
| | RBRYBRXR-aa | 251 |
| | RXRILFQYRXR-aa | 252 |
| | RBRILFQYRBR-aa | 253 |
| | RXRILFQYRBR-aa | 254 |
| | RBRILFQYRXR-aa | 255 |
| | RBRRBRRBR-aa | 256 |
| | RXRRBRRXR-aa | 257 |
| | RXRRBRRBR-aa | 258 |
| | RXRRXRRBR-aa | 259 |
| | RBRRXRRBR-aa | 260 |
| | RBRRXRRXR-aa | 261 |
| | RBRRBRRXR-aa | 262 |
| | RXRYBRXRRXR-aa | 263 |
| | RXRRXRYBRXR-aa | 264 |
| | RXRILFQYRXRRXR-aa | 265 |
| | RXRRXRILFQYRXR-aa | 266 |
| | RXRYBRXRYBRXR-aa | 267 |
| | RXRILFQYRXRILFQYRXR-aa | 268 |
| | RXRILFQYRXRYBRXR-aa | 269 |
| | RXRYBRXRILFQYRXR-aa | 270 |
| | RBRYBRBRRBR-aa | 271 |
| | RBRRBRYBRBR-aa | 272 |
| | RBRILFQYRBRRBR-aa | 273 |
| | RBRRBRILFQYRBR-aa | 274 |
| | RBRYRBRYBRBR-aa | 275 |
| | RBRILFQYRBRILFQYRBR-aa | 276 |
| | RBRYBRBRILFQYRBR-aa | 277 |
| | RBRILFQYRBRYBRBR-aa | 278 |
| | RXRYBRBRRXR-aa | 279 |
| | RXRRBRYBRXR-aa | 280 |
| | RXRILFQYRBRRXR-aa | 281 |
| | RXRRBRILFQYRXR-aa | 282 |
| | RXRYBRBRYBRXR-aa | 283 |
| | RXRILFQYRBRILFQYRXR-aa | 284 |
| | RXRYBRBRILFQYRXR-aa | 285 |
| | RXRILFQYRBRYBRXR-aa | 286 |
| | RXRYBRBRRBR-aa | 287 |
| | RXRRBRYBRBR-aa | 288 |
| | RXRILFQYRBRRBR-aa | 289 |
| | RXRRBRILFQYRBR-aa | 290 |
| | RXRYBRBRYBRBR-aa | 291 |
| | RXRILFQYRBRILFQYRBR-aa | 292 |
| | RXRYBRBRILFQYRBR-aa | 293 |
| | RXRILFQYRBRYBRBR-aa | 294 |
| | RXRYBRXRRBR-aa | 295 |
| | RXRRXRYBRBR-aa | 296 |
| | RXRILFQYRXRRBR-aa | 297 |
| | RXRRXRILFQYRBR-aa | 298 |
| | RXRYBRXRYBRBR-aa | 299 |
| | RXRILFQYRXRILFQYRBR-aa | 300 |
| | RXRYBRXRILFQYRBR-aa | 301 |
| | RXRILFQYRXRYBRBR-aa | 302 |
| | RBRYBRXRRBR-aa | 303 |
| | RBRRXRYBRBR-aa | 304 |
| | RBRILFQYRXRRBR-aa | 305 |
| | RBRRXRILFQYRBR-aa | 306 |
| | RBRYBRXRYBRBR-aa | 307 |
| | RBRILFQYRXRILFQYRBR-aa | 308 |
| | RBRYBRXRILFQYRBR-aa | 309 |
| | RBRILFQYRXRYBRBR-aa | 310 |
| | RBRYBRXRRXR-aa | 311 |
| | RBRRXRYBRXR-aa | 312 |
| | RBRILFQYRXRRXR-aa | 313 |
| | RBRRXRILFQYRXR-aa | 314 |
| | RBRYBRXRYBRXR-aa | 315 |
| | RBRILFQYRXRILFQYRXR-aa | 316 |
| | RBRYBRXRILFQYRXR-aa | 317 |
| | RBRILFQYRXRYBRXR-aa | 318 |
| | RBRYBRBRRXR-aa | 319 |
| | RBRRBRYBRXR-aa | 320 |
| | RBRILFQYRBRRXR-aa | 321 |
| | RBRRBRILFQYRXR-aa | 322 |
| | RBRYBRBRYBRXR-aa | 323 |
| | RBRILFQYRBRILFQYRXR-aa | 324 |
| | RBRYBRBRILFQYRXR-aa | 325 |
| | RBRILFQYRBRYBRXR-aa | 326 |
| | RXRRBRRXRILFQYRXRBRXR-aa | 327 |
| | YGRKKRRQRRRP-aa | 328 |
| | RXRRXRRXRRXRXBASSLNIAXC-aa | 329 |
| | RXRRBRRXRILFQYRXRBRXRBASSLNIAXC-aa | 330 |
| | RXRRBRRXRASSLNIARXRBRXRBC-aa | 331 |
| | RXRRBRRXRRBRXBASSLNIA-aa | 332 |
| | THRPPMWSPVWP-aa | 333 |
| | HRPPMWSPVWP-aa | 334 |
| | THRPPMWSPV-aa | 335 |
| | THRPPMWSP-aa | 336 |
| | THRPPMWSPVFP-aa | 337 |
| | THRPPMWSPVYP-aa | 338 |
| | THRPPMWSPAWP-aa | 339 |
| | THRPPMWSPLWP-aa | 340 |
| | THRPPMWSPIWP-aa | 341 |
| | THRPPMWTPVVWP-aa | 342 |
| | THRPPMFSPVWP-aa | 343 |
| | THRPPMWS-aa | 344 |
| | HRPPMWSPVW-aa | 345 |
| | THRPPMYSPVWP-aa | 346 |
| | THRPPnleWSPVWP-aa | 347 |
| | THKPPMWSPVWP-aa | 348 |
| | SHRPPMWSPVWP-aa | 349 |
| | STFTHPR-aa | 350 |
| | YDIDNRR-aa | 351 |
| | AYKPVGR-aa | 352 |
| | HAIYPRH-aa | 353 |
| | HTPNSTH-aa | 354 |
| | ASSPVHR-aa | 355 |
| | SSLPLRK-aa | 356 |
| | KKRS-aa | 357 |
| | KRSK-aa | 358 |
| | KKRSK-aa | 359 |
| | KSRK-aa | 360 |
| | SRKR-aa | 361 |
| | RKRK-aa | 362 |
| | KSRKR-aa | 363 |
| | QHPPWRV-aa | 364 |
| | THPPTTH-aa | 365 |
| | YKHTPTT-aa | 366 |
| | QGMHRGT-aa | 367 |
| | SRKRK-aa | 368 |
| | KSRKRK-aa | 369 |
| | PKKKRKV-aa | 370 |
| | GKKRSKV-aa | 371 |
| | KSRKRKL-aa | 372 |
| | HSPSKIP-aa | 373 |
| | HMATFHY-aa | 374 |
| | AQPNKFK-aa | 375 |
| | NLTRLHT-aa | 376 |
| | KKKR-aa | 377 |
| | KKRK-aa | 378 |
| | KKKRK-aa | 379 |
| | RRRRRRQIKIWFQNRRMKWKKGGC-aa | 380 |
| | RRRRRRRQIKIWFQNRRMKWKKGGC-aa | 381 |
| | RQIKIWFQNRRMKWKKGGC-aa | 382 |
| | RRRRRRRQIKIWFQNRRMKWKKC-aa | 383 |
| | RXRRXRRXRRQIKIWFQNRRMKWKKGGC-aa | 384 |
| | RRRRRRRQIKILFQNRXRXRXRXC-aa | 385 |
| | RXRRXRRXRRXRC-aa | 386 |
| | RXRRXRRXRRXRXC-aa | 387 |
| | RXRRXRRXRIKILFQNRRMKWKKGGC-aa | 388 |
| | RXRRXRRXRIKILFQNRRMKWKKC-aa | 389 |
| | RXRRXRRXRIKILFQNRMKWKKC-aa | 390 |
| | RXRRXRRXRIKILFQNXRMKWKKC-aa | 391 |
| | RXRRXRRXRIKILFQNHRMKWKKC-aa | 392 |
| | RXRRXRRXRIKILFQNXRMKWKAC-aa | 393 |
| | RXRRXRRXRIKILFQNXRMKWHKAC-aa | 394 |
| | RXRRXRRXRIKILFQNXRMKWHRC-aa | 395 |
| | RXRXRXRXRIKILFQNRRMKWKKC-aa | 396 |
| | RARARARARIKILFQNRRMKWKKC-aa | 397 |
| | RXRRXRRXRIXILFQNXRMKWHKAC-aa | 398 |
| | RXRRXRRXRIHILFQNXRMKWHKAC-aa | 399 |
| | RXRRXRRXRIRILFQNXRMKWHKAC-aa | 400 |
| | RXRRXRRXRIXILFQYXRMKWHKAC-aa | 401 |
| | RXRRXRRXRLYSPLSFQXRMKWHKAC-aa | 402 |
| | RXRRXRRXRISILFQYXRMKWHKAC-aa | 403 |
| | RXRRXRRXRILFQYXRMKWHKAC-aa | 404 |
| | RXRRXRIXILFQYXRMKWHKAC-aa | 405 |
| | RXRRARRXRIHILFQYXRMKWHKAC-aa | 406 |
| | RARRXRRARIHILFQYXRMKWHKAC-aa | 407 |
| | RXRRXRRXRIHILFQYXRMKWHKAC-aa | 408 |
| | RXRRXRRXRIKILFQNRRMKWHK-aa | 409 |
| | RXRRXRRXRIKILFQNXRMKWHK-aa | 410 |
| | RXRRXRRXRIXILFQNRRMKWHK-aa | 411 |
| | RXRRXRRXRIXILFQNXRMKWHK-aa | 412 |
| | RXRRXRRXRIHILFQNRRMKWHK-aa | 413 |
| | RXRRXRRXRIHILFQNXRMKWHK-aa | 414 |
| | RXRRXRRXRIRILFQNRRMKWHK-aa | 415 |
| | RXRRXRRXRIRILFQNXRMKWHK-aa | 416 |
| | RXRRXRRXRIILFQNRRMKWHK-aa | 417 |
| | RXRRXRRXRIILFQNXRMKWHK-aa | 418 |
| | RXRRXRRXRKILFQNRRMKWHK-aa | 419 |
| | RXRRXRRXRKILFQNXRMKWHK-aa | 420 |
| | RXRRXRRXRXILFQNRRMKWHK-aa | 421 |
| | RXRRXRRXRXILFQNXRMKWHK-aa | 422 |
| | RXRRXRRXRHILFQNRRMKWHK-aa | 423 |
| | RXRRXRRXRHILFQNXRMKWHK-aa | 424 |
| | RXRRXRRXRRILFQNRRMKWHK-aa | 425 |
| | RXRRXRRXRRILFQNXRMKWHK-aa | 426 |
| | RXRRXRRXRILFQNRRMKWHK-aa | 427 |
| | RXRRXRRXRILFQNXRMKWHK-aa | 428 |
| | RXRRXRRXRIKILFQYRRMKWHK-aa | 429 |
| | RXRRXRRXRIKILFQYXRMKWHK-aa | 430 |
| | RXRRXRRXRIXILFQYRRMKWHK-aa | 431 |
| | RXRRXRRXRIXILFQYXRMKWHK-aa | 432 |
| | RXRRXRRXRIHILFQYRRMKWHK-aa | 433 |
| | RXRRXRRXRIHILFQYXRMKWHK-aa | 434 |
| | RXRRXRRXRIRILFQYRRMKWHK-aa | 435 |
| | RXRRXRRXRIRILFQYXRMKWHK-aa | 436 |
| | RXRRXRRXRIILFQYRRMKWHK-aa | 437 |
| | RXRRXRRXRIILFQYXRMKWHK-aa | 438 |
| | RXRRXRRXRKILFQYRRMKWHK-aa | 439 |
| | RXRRXRRXRKILFQYXRMKWHK-aa | 440 |
| | RXRRXRRXRXILFQYRRMKWHK-aa | 441 |
| | RXRRXRRXRXILFQYXRMKWHK-aa | 442 |
| | RXRRXRRXRHILFQYRRMKWHK-aa | 443 |
| | RXRRXRRXRHILFQYXRMKWHK-aa | 444 |
| | RXRRXRRXRRILFQYRRMKWHK-aa | 445 |
| | RXRRXRRXRRILFQYXRMKWHK-aa | 446 |
| | RXRRXRRXRILFQYRRMKWHK-aa | 447 |
| | RXRRXRRXRILFQYXRMKWHK-aa | 448 |
| | RARRAR-aa | 449 |
| | RARRARRAR-aa | 450 |
| | RARRARRARRAR-aa | 451 |
| | RXRRXRI-aa | 452 |
| | RXRRARRXR-aa | 453 |
| | RARRXRRAR-aa | 454 |
| | RRMKWHK-aa | 455 |
| | XRMKWHK-aa | 456 |
| | XXXXXXXXXXXXXXILFQXXRMKWHK-aa | 457 |
| | RRRRRRRQIKILFQNPKKKRKVGGC-aa | 458 |
| | HHFFRRRRRRRRRFFC-aa | 459 |
| | HHHHHHRRRRRRRRRFFC-aa | 460 |
| | HHHHHHFFRRRRRRRRRFFC-aa | 461 |
| | HHHHHXXRRRRRRRRRFFC-aa | 462 |
| | HHHHHHXXFFRRRRRRRRRFFC-aa | 463 |
| | HHHXRRRRRRRRRFFXHHHC-aa | 464 |
| | XRWKWHK-aa | 465 |
| | RXRARXR-aa | 466 |
| | RXRXRXR-aa | 467 |
| | RARXRAR-aa | 468 |
| | RXRAR-aa | 469 |
| | XXXXXXXXXXXXXXILFQXXHMKWHK-aa | 470 |
| | XXXXXXXXXXXXXXILFQXXRWKWHK-aa | 471 |
| | XXXXXXXXXXXXXXILFQXXHWKWHK-aa | 472 |
| | XXXXXXXXXXXXXXILFQXRXRARXR-aa | 473 |
| | XXXXXXXXXXXXXXILFQXRXRXRXR-aa | 474 |
| | XXXXXXXXXXXXXXILFQXRXRRXR-aa | 475 |
| | XXXXXXXXXXXXXXILFQXRARXRAR-aa | 476 |
| | XXXXXXXXXXXXXXILIQXXRMKWHK-aa | 477 |
| | XXXXXXXXXXXXXXILIQXXHMKWHK-aa | 478 |
| | XXXXXXXXXXXXXXILIQXXRWKWHK-aa | 479 |
| | XXXXXXXXXXXXXXILIQXRXRARXR-aa | 480 |
| | XXXXXXXXXXXXXXILIQXRXRRXR-aa | 481 |
| | XXXXXXXXXXXXXXILIQXRARXRAR-aa | 482 |
| | XXXXXXXXXXXXXXILIQXRXRAR-aa | 483 |
| | XXXXXXXXXXXXXXILFQXRXRAR-aa | 484 |
| | XXXXXXXXXXXXXXILIQXXHWKWHK-aa | 485 |
| | XXXXXXXXXXXXXXILIQXRXRXRXR-aa | 486 |
| | RXRRARRXRRARXA-aa | 487 |
| | RXRRARRXRILFQYXHMKWHKAC-aa | 488 |
| | RXRRARRXRILFQYXRMKWHKAC-aa | 489 |
| | RXRRARRXRILFQYXRWKWHKAC-aa | 490 |
| | RARRXRRARILFQYXRMKWHKAC-aa | 491 |
| | RXRRARRXRILFQYXHWKWHKAC-aa | 492 |
| | RXRRARRXRILFQYRXRARXRAC-aa | 493 |
| | RXRRARRXRILFQYRXRXRXRAC-aa | 494 |
| | RXRRARRXRILIQYXRMKWHKAC-aa | 495 |
| | RXRRXRILFQYRXRRXRC-aa | 496 |
| | RXRRXRILFQYRXRRXRCYS-aa | 497 |
| | RARRXRRARILFQYRARXRARAC-aa | 498 |
| | RARRXRRARILFQYRXRARXRAC-aa | 499 |
| | RARRXRRARILFQYRXRRXRAC-aa | 500 |
| | RARRXRRARILFQYRXRARXAC-aa | 501 |
| | RXRRARRXRILFQYRXRRXRAC-aa | 502 |
| | RXRRARRXRILFQYRXRARXAC-aa | 503 |
| | RXRRARRXRIHILFQNXRMKWHKAC-aa | 504 |
| | RXRRARRXRRARXAC-aa | 505 |
| | RXRRARRXRILFQYXHMKWHK-aa | 506 |
| | RXRRARRXRILFQYXRMKWHK-aa | 507 |
| | RXRRARRXRILFQYXRWKWHK-aa | 508 |
| | RXRRARRXRILFQYRXRARXR-aa | 509 |
| | RXRRARRXRILFQYRXRXRXR-aa | 510 |
| | RXRRARRXRILFQYRXRRXR-aa | 511 |
| | RXRRARRXRILFQYRARXRAR-aa | 512 |
| | RXRRARRXRILFQYRXRAR-aa | 513 |
| | RXRRARRXRILIQYXHMKWHK-aa | 514 |
| | RXRRARRXRILIQYXRMKWHK-aa | 515 |
| | RXRRARRXRILIQYXRWKWHK-aa | 516 |
| | RXRRARRXRILIQYRXRARXR-aa | 517 |
| | RXRRARRXRILIQYRXRXRXR-aa | 518 |
| | RXRRARRXRILIQYRXRRXR-aa | 519 |
| | RXRRARRXRILIQYRARXRAR-aa | 520 |
| | RXRRARRXRILIQYRXRAR-aa | 521 |
| | RARRXRRARILFQYXHMKWHK-aa | 522 |
| | RARRXRRARILFQYXRMKWHK-aa | 523 |
| | RARRXRRARILFQYXRWKWHK-aa | 524 |
| | RARRXRRARILFQYRXRARXR-aa | 525 |
| | RARRXRRARILFQYRXRXRXR-aa | 526 |
| | RARRXRRARILFQYRXRRXR-aa | 527 |
| | RARRXRRARILFQYRARXRAR-aa | 528 |
| | RARRXRRARILFQYRXRAR-aa | 529 |
| | RARRXRRARILIQYXHMKWHK-aa | 530 |
| | RARRXRRARILIQYXRMKWHK-aa | 531 |
| | RARRXRRARILIQYXRWKWHK-aa | 532 |
| | RARRXRRARILIQYRXRARXR-aa | 533 |
| | RARRXRRARILIQYRXRXRXR-aa | 534 |
| | RARRXRRARILIQYRXRRXR-aa | 535 |
| | RARRXRRARILIQYRARXRAR-aa | 536 |
| | RARRXRRARILIQYRXRAR-aa | 537 |
| | RXRRXRILFQYXHMKWHK-aa | 538 |
| | RXRRXRILFQYXRMKWHK-aa | 539 |
| | RXRRXRILFQYXRWKWHK-aa | 540 |
| | RXRRXRILFQYRXRARXR-aa | 541 |
| | RXRRXRILFQYRXRXRXR-aa | 542 |
| | RXRRXRILFQYRXRRXR-aa | 543 |
| | RXRRXRILFQYRARXRAR-aa | 544 |
| | RXRRXRILFQYRXRAR-aa | 545 |
| | RXRRXRILIQYXHMKWHK-aa | 546 |
| | RXRRXRILIQYXRMKWHK-aa | 547 |
| | RXRRXRILIQYXRWKWHK-aa | 548 |
| | RXRRXRILIQYRXRARXR-aa | 549 |
| | RXRRXRILIQYRXRXRXR-aa | 550 |
| | RXRRXRILIQYRXRRXR-aa | 551 |
| | RXRRXRILIQYRARXRAR-aa | 552 |
| | RXRRXRILIQYRXRAR-aa | 553 |
| | PRPXXXXXXXXXXXPRG-aa | 554 |
| | RRMKWKK-aa | 555 |
| | CKDEPQRRSARLSAKPAPPKPEPKPKKAPAKK-aa | 556 |
| | RKKRRQRR-aa | 557 |
| | RKKRRQR-aa | 558 |
| | KKRRQRRR-aa | 559 |
| | AKKRRQRRR-aa | 560 |
| | RAKRRQRRR-aa | 561 |
| | RKARRQRRR-aa | 562 |
| | RKKARQRRR-aa | 563 |

Sequences assigned to SEQ ID NOs do not include the linkage portion “aa”. In the amino acid sequence, A represents Alanine, B represents β-Alanine (also represented as β-Ala or β), F represents Phenylalanine, G represents Glycine, L represents Leucine, R represents Arginine, and X represent 6-Aminohexanoic acid (also represented as Ahx or a), C represents Cysteine, D represents Aspartic acid, E represents Glutamic acid, H represents Histidine, I represents Isoleucine, K represents Lysine, M represents Methionine, N represents Asparagine, P represents Proline, Q represents Glutamine, S represents Serine, T represents Threonine, V represents Valine, W represents Tryptophan, Y represents Tyrosine, and nle represents norleucine. In some embodiments, an antisense oligomer, or a pharmaceutically acceptable salt thereof, disclosed herein is a conjugate of D-Pep 1.9b wherein the carboxy-terminal linker “-aa” is with isoglutamine (IsoGln).

Each peptide can comprise an unmodified amino terminus, or the amino-terminal capped with an acetyl, benzoyl or stearoyl group (i.e., an acetyl amide, benzoyl amide or stearoyl amide) and Y is NH—(CHR)—C(O)— wherein n is 2 to 7 and each R is independently, at each occurrence, hydrogen or methyl. Each of the above sequences may comprise an unmodified amino terminus or an amino terminus capped with an acetyl, benzoyl, or stearoyl group. In some embodiments, the carboxy-terminal linker “-aa” may additionally be an Isoglutamine amino acid residue (IsoGln) linking the carrier peptide to the oligomer by an amide bond between the carboxyl of IsoGln and the oligomer and an amide bond between the amino of IsoGln and the carboxy terminus of the peptide. Examples of use of IsoGln as a linker can be found in, for example, WO/2022/171972, which is incorporated by reference in its entirety.

### VI. Pharmaceutical Compositions

The present disclosure also provides for the formulation and delivery of the disclosed antisense oligomers, or pharmaceutically acceptable salts thereof. Accordingly, an aspect of the present disclosure is a pharmaceutical composition comprising antisense oligomers, or pharmaceutically acceptable salts thereof, as disclosed herein and a pharmaceutically acceptable carrier.

Effective delivery of the antisense oligomers, or of pharmaceutically acceptable salts thereof, to the target nucleic acid is an important aspect of treatment. Routes of antisense oligomer, or pharmaceutically acceptable salt thereof, delivery include, but are not limited to, various systemic routes, including oral and parenteral routes, e.g., intravenous, subcutaneous, intraperitoneal, and intramuscular, as well as inhalation, transdermal, and topical delivery. The appropriate route may be determined by one of skill in the art, as appropriate to the condition of the subject under treatment. For example, an appropriate route for delivery of an antisense oligomer, or a pharmaceutically acceptable salt thereof, in the treatment of a viral infection of the skin is topical delivery, while the delivery of an antisense oligomer, or of a pharmaceutically acceptable salt thereof, for the treatment of a viral respiratory infection can be intravenous or by inhalation. The antisense oligomer, or a pharmaceutically acceptable salt thereof, may also be delivered directly to any particular site of viral infection.

The antisense oligomer, or a pharmaceutically acceptable salt thereof, can be administered in any convenient vehicle which is physiologically and/or pharmaceutically acceptable. Such a composition can include any of a variety of standard pharmaceutically acceptable carriers employed by those of ordinary skill in the art. Examples include, but are not limited to, saline, phosphate-buffered saline (PBS), water (e.g., sterile water for injection), aqueous ethanol, emulsions such as oil/water emulsions or triglyceride emulsions, tablets, and capsules. The choice of a suitable physiologically acceptable carrier will vary dependent upon the chosen mode of administration.

The compounds disclosed herein (e.g., an antisense oligomer, or a pharmaceutically acceptable salt thereof,) can generally be utilized as the free acid or free base. Alternatively, the compounds disclosed herein may be used in the form of acid or base addition salts. Acid addition salts of the free amino compounds may be prepared by methods well known in the art and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of Formulae (I), (IA), (II), (III), and/or (IV) is intended to encompass any and all acceptable salt forms.

In addition, prodrugs are also included within the context of this disclosure. Prodrugs are any covalently bonded carriers that release a compound of structure (I) in vivo when such a prodrug is administered to a patient. Prodrugs are generally prepared by modifying functional groups in a way such that the modification is cleaved, either by routine manipulation or in vivo, yielding the parent compound. Prodrugs include, for example, compounds of this disclosure wherein hydroxy, amine, or sulfhydryl groups are bonded to any group that, when administered to a patient, cleaves to form the hydroxy, amine, or sulfhydryl groups. Thus, representative examples of prodrugs include (but are not limited to) acetate, formate, and benzoate derivatives of alcohol and amine functional groups of the compounds of structure (I). Further, in the case of a carboxylic acid (—COOH), esters may be employed, such as methyl esters, ethyl esters, and the like.

### VII. Methods of Making

Preparation of Oligomers with Basic Nitrogen Internucleoside Linkers

Morpholino subunits, the modified intersubunit linkages, and oligomers comprising the same can be prepared as described, for example, in U.S. Pat. Nos. 5,185,444, and 7,943,762, each of which incorporated herein by reference in its entirety. The morpholino subunits can be prepared according to the following general Reaction Scheme 1.

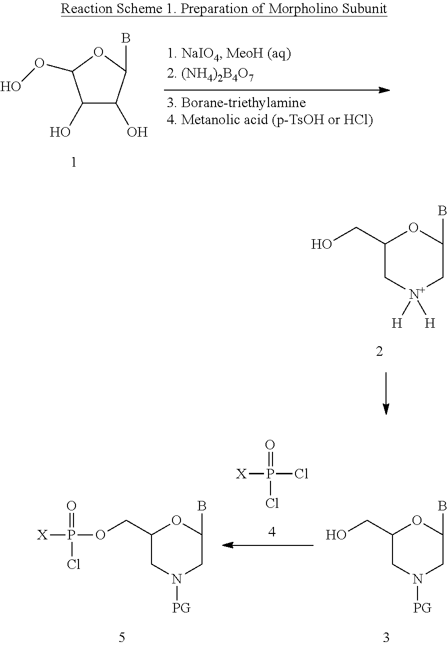

Reaction of 3 with the activated phosphorus compound 4 results in morpholino subunits having the desired linkage moiety 5. Compounds of structure 4 can be prepared using any number of methods known to those of skill in the art. For example, such compounds may be prepared by reaction of the corresponding amine and phosphorus oxychloride. In this regard, the amine starting material can be prepared using any method known in the art, for example those methods described in the Examples and in U.S. Pat. No. 7,943,762.

Compounds of structure 5 can be used in solid-phase automated oligomer synthesis for the preparation of oligomers comprising the intersubunit linkages. Such methods are well known in the art. Briefly, a compound of structure 5 may be modified at the 5′ end to contain a linker to a solid support. For example, compound 5 may be linked to a solid support by a linker. Once supported, the protecting group (e.g., trityl) is removed and the free amine is reacted with an activated phosphorus moiety of a second compound of structure 5. This sequence is repeated until the desired length of oligo is obtained. The protecting group in the terminal 5′ end may either be removed or left on if a 5′-modification is desired.

The preparation of modified morpholino subunits and morpholino oligomers are described in more detail in the Examples. The morpholino oligomers containing any number of modified linkages may be prepared using methods described herein, methods known in the art and/or incorporated herein by reference. Also described in the examples are global modifications of morpholino oligomers prepared as previously described (see e.g., PCT publication WO 2008/036127).

Synthesis of PMO, PMO+, PPMO, and PMO-X containing further linkage modifications as described herein was done using methods known in the art and described in pending U.S. Pat. Nos. 8,299,206 and 8,076,476 and PCT publication numbers WO 2009/064471, WO 2011/150408 and WO 2012/150960, which are hereby incorporated herein by reference in their entirety.

PMO with a 3′ trityl modification are synthesized essentially as described in PCT publication number WO 2009/064471 with the exception that the detritylation step is omitted.

### VIII. Methods of Treatment

Provided herein is a method of reducing the expression of a pre-mRNA of the human UMOD gene and/or protein using the antisense oligomers, or pharmaceutically acceptable salts thereof, of the present disclosure for therapeutic purposes (e.g., treating subjects with chronic kidney disease such as ADTKD-UMOD). The method comprises administering to a patient in need thereof a therapeutically effective amount of an antisense oligomer, or a pharmaceutically acceptable salt thereof, disclosed herein.

In an embodiment, the method comprises administering to a patient in need thereof a therapeutically effective amount of a pharmaceutical composition, wherein the composition comprises an antisense oligomer, or a pharmaceutically acceptable salt thereof, and a pharmaceutically acceptable carrier. In an embodiment, the disease is chronic kidney disease (CKD). In an embodiment, the disease is associated with aberrant expression of uromodulin protein (UMOD). In an embodiment, the disease is a uromodulin-associated renal disease. In an embodiment, the disease is an autosomal dominant renal disorder. In an embodiment, the autosomal dominant renal disorder is an autosomal dominant tubulointerstitial kidney disease (ADTKD). In an embodiment, the disease is uromodulin associated autosomal dominant tubulointerstitial kidney disease (AKTD-UMOD), which is also known as uromodulin kidney disease (UKD). In an embodiment, the method of treatment disclosed herein improves clinical symptoms associated with or manifestations of chronic kidney disease (e.g., ADTKD-UMD) including gout (e.g., early-onset gout), urinary tract infection(s), hyperuricemia, kidney stones, reduced urine concentrating and creatinine clearance, hypertension, etc.

In an embodiment, the method of treatment disclosed herein slows or arrests progressive of loss of renal function. In an embodiment, the method of treatment disclosed herein improves kidney function. In an embodiment, the method of treatment disclosed herein reverses kidney disease including end stage kidney disease. In an embodiment, the subject is a mammal. In an embodiment, the subject is a non-human primate. In an embodiment, the subject is a human. In some embodiments, the subject is a cell. In some embodiments, the cell is a diseased cell.

In certain embodiments, the disease is caused by a mutation in the UMOD gene. In some embodiments, the mutation is a missense mutation. In some embodiments, the mutation is a point mutation targeting at least one cysteine residue. In some embodiments, missense mutations cause misfolding in the UMOD protein, leading to retention in the endoplasmic reticulum (ER). In certain embodiments, mutations are missense changes. In some embodiments, a number of mutations cluster around cysteine residues in UMOD, causing protein misfolding. In some embodiments, accumulation of mutant UMOD induces ER stress, unfolded protein response (UPR), mitochondrial dysfunction, defective protein homeostasis, and autophagy in TAL cells resulting in apoptosis of TAL epithelial cells, inflammatory lesions, fibrosis, tubular atrophy, cystic dilation, and progressive loss of kidney function. In some embodiments, mutant UMOD also inhibits NKCC2 activity in the TAL, leading to lower urate excretion, hyperuricemia, and gout.

In some embodiments, the antisense oligomer, or a pharmaceutically acceptable salt thereof, comprises a nucleotide sequence of sufficient length and complementarity to specifically hybridize to a region within the pre-mRNA of the human UMOD gene, wherein binding of the antisense oligomer, or of a pharmaceutically acceptable salt thereof, to the region induces exon skipping in the human UMOD gene pre-mRNA, thereby triggering nonsense-mediated decay of UMOD in a cell and/or tissue of the subject. Exemplary antisense targeting sequences are shown in Table 5 and Table 6 herein.

Also included are antisense oligomers, or pharmaceutically acceptable salts thereof, for use in the preparation of a medicament for the treatment of chronic kidney disease (CKD).

Included are antisense oligomers, or pharmaceutically acceptable salts thereof, for use in the preparation of a medicament for the treatment of a kidney disease associated with aberrant expression of uromodulin protein (UMOD). Included are antisense oligomers, or pharmaceutically acceptable salts thereof, for use in the preparation of a medicament for the treatment of a uromodulin-associated renal disease. Included are antisense oligomers, or pharmaceutically acceptable salts thereof, for use in the preparation of a medicament for the treatment of an autosomal dominant renal disorder. Included are antisense oligomers, or pharmaceutically acceptable salts thereof, for use in the preparation of a medicament for the treatment of an autosomal dominant tubulointerstitial kidney disease (ADTKD). Included are antisense oligomers, or pharmaceutically acceptable salts thereof, for use in the preparation of a medicament for the treatment of uromodulin associated autosomal dominant tubulointerstitial kidney disease (AKTD-UMOD), which is also known as uromodulin kidney disease (UKD).

In some embodiments, the antisense oligomers, or pharmaceutically acceptable salts thereof, disclosed herein comprise a nucleotide sequence of sufficient length and complementarity to specifically hybridize to a target region within the pre-mRNA of the human UMOD gene, wherein binding of the antisense oligomer, or of a pharmaceutically acceptable salt thereof, to the target region decreases the expression of UMOD.

In some embodiments of the method of treating chronic kidney disease, including uromodulin-associated renal disease kidney disease such as kidney disease associated with aberrant expression of uromodulin protein, an autosomal dominant renal disorder (e.g., autosomal dominant tubulointerstitial kidney disease such as ADTKD-UMOD) or the medicament for the treatment of chronic kidney disease, including uromodulin-associated renal disease kidney disease such as kidney disease associated with aberrant expression of uromodulin protein, an autosomal dominant renal disorder (e.g., autosomal dominant tubulointerstitial kidney disease such as ADTKD-UMOD), the antisense oligomer compound, or a pharmaceutically acceptable salt thereof, comprises a non-natural chemical backbone and a targeting sequence of 13 to 30 bases in length that is complementary to a target region within a pre-mRNA of the human uromodulin (UMOD) gene (SEQ ID NO: 1). In certain embodiments, the target region is an intron/exon junction or an exon internal region of the human UMOD gene pre-mRNA.

In some embodiments of the method of treating chronic kidney disease, including uromodulin-associated renal disease kidney disease such as kidney disease associated with aberrant expression of uromodulin protein, an autosomal dominant renal disorder (e.g., autosomal dominant tubulointerstitial kidney disease such as ADTKD-UMOD) or the medicament for the treatment of chronic kidney disease, including uromodulin-associated renal disease kidney disease such as kidney disease associated with aberrant expression of uromodulin protein, an autosomal dominant renal disorder (e.g., autosomal dominant tubulointerstitial kidney disease such as ADTKD-UMOD), the antisense oligomer compound, or a pharmaceutically acceptable salt thereof, comprises a non-natural chemical backbone and a targeting sequence of 13 to 30 bases in length that is complementary to a target region within a pre-mRNA of the human uromodulin (UMOD) gene (SEQ ID NO: 1). In certain embodiments, the target region is an intron/exon junction or an exon internal region of exon 2 (SEQ ID NO: 2), exon 5 (SEQ ID NO: 3), exon 6 (SEQ ID NO: 4), exon 8 (SEQ ID NO: 5), or exon 9 (SEQ ID NO: 6) of the human UMOD gene pre-mRNA.

In some embodiments of the method of treating chronic kidney disease, including uromodulin-associated renal disease kidney disease such as kidney disease associated with aberrant expression of uromodulin protein, an autosomal dominant renal disorder (e.g., autosomal dominant tubulointerstitial kidney disease such as ADTKD-UMOD) or the medicament for the treatment of chronic kidney disease, including uromodulin-associated renal disease kidney disease such as kidney disease associated with aberrant expression of uromodulin protein, an autosomal dominant renal disorder (e.g., autosomal dominant tubulointerstitial kidney disease such as ADTKD-UMOD), the antisense oligomer compound, or pharmaceutically acceptable salt thereof, comprises a non-natural chemical backbone and a targeting sequence of 13 to 30 bases in length that is complementary to a target region within a pre-mRNA of the human uromodulin (UMOD) gene (SEQ ID NO: 1). In certain embodiments, the target region is an intron/exon junction or an exon internal region of the human UMOD gene pre-mRNA, and wherein the antisense oligomer, or a pharmaceutically acceptable salt thereof, is an antisense oligomer, or a pharmaceutically acceptable salt thereof, of Formula (I), Formula (IA), Formula (II), Formula (III), or Formula (IV). As noted above, “ADTKD-UMOD” refers to uromodulin associated autosomal dominant tubulointerstitial kidney disease (ADTKD-UMOD or UKD), a human autosomal recessive disease that is often characterized by intracellular aggregation and accumulation of UMOD protein in affected individuals. In certain embodiments, a subject has aberrant expression and/or aggregation of UMOD protein in kidney tissues and cells.

Certain embodiments relate to methods of reducing UMOD expression in a cell, tissue, and/or subject, as described herein. In some instances, UMOD expression is reduced by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% relative to a control, for example, a control cell/subject, a control composition without the antisense oligomer, or without a pharmaceutically acceptable salt thereof, the absence of treatment, and/or an earlier time-point. In some instances, UMOD expression is reduced by about 5% to about 100%, relative to a control. In some instances, UMOD expression is reduced by about 10% to about 95%, relative to a control. In some instances, UMOD expression is reduced by about 20% to about 95%, relative to a control. In some instances, UMOD expression is reduced by about 30% to about 95%, relative to a control. In some instances, UMOD expression is reduced by about 30% to about 90%, relative to a control. In some instances, UMOD expression is reduced by about 40% to about 90%, relative to a control. In some instances, UMOD expression is reduced by about 50% to about 90%, relative to a control. In some instances, UMOD expression is reduced by about 45% to about 85%, relative to a control. In some instances, UMOD expression is reduced by about 50% to about 85%, relative to a control. In some instances, UMOD expression is reduced by about 55% to about 85%, relative to a control. In some instances, UMOD expression is reduced by about 60% to about 85%, relative to a control. In some instances, UMOD expression is reduced by about 65% to about 85%, relative to a control. In some instances, UMOD expression is reduced by about 50% to about 80%, relative to a control. In some instances, UMOD expression is reduced by about 60% to about 80%, relative to a control. In some instances, UMOD expression is reduced by about 50% to about 75%, relative to a control. In some instances, UMOD expression is reduced by about 50% to about 70%, relative to a control. In some instances, UMOD expression is reduced by about 60% to about 75%, relative to a control. In some instances, UMOD expression is reduced by about 65% to about 75%, relative to a control.

In some instances, UMOD expression is reduced by about 30% to about 75%, relative to a control. In some instances, UMOD expression is reduced by about 30% to about 80%, relative to a control. In some instances, UMOD expression is reduced by about 85% to about 75%, relative to a control. In some instances, UMOD expression is reduced by about 40% to about 75%, relative to a control. In some instances, UMOD expression is reduced by about 40% to about 80%, relative to a control. In some instances, UMOD expression is reduced by about 40% to about 85%, relative to a control. In some instances, UMOD expression is reduced by about 20% to about 75%, relative to a control. In some instances, UMOD expression is reduced by about 20% to about 80%, relative to a control. In some instances, UMOD expression is reduced by about 20% to about 85%, relative to a control.

Also included are methods of reducing one or more symptoms of ADTKD-UMOD in a subject in need thereof. Examples include symptoms such as elevated blood creatinine levels, interstitial fibrosis, tubular atrophy, gout, hyperuricemia, and end stage kidney disease (ESKD).

The antisense oligomers, or pharmaceutically acceptable salts thereof, of the disclosure can be administered to subjects to treat (prophylactically or therapeutically) ADTKD-UMOD. In conjunction with such treatment, pharmacogenomics (i.e., the study of the relationship between an individual's genotype and that individual's response to a foreign compound or drug) may be considered. Differences in the metabolism of therapeutics can lead to severe toxicity or therapeutic failure by altering the relation between dose and blood concentration of the pharmacologically active drug.

Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Effective delivery of the antisense oligomer, or of a pharmaceutically acceptable salt thereof, to the target nucleic acid is one aspect of treatment. Routes of antisense oligomer, or of a pharmaceutically acceptable salts thereof, delivery include, but are not limited to, various systemic routes, including oral and parenteral routes, e.g., intravenous, subcutaneous, intraperitoneal, and intramuscular, as well as inhalation, transdermal, and topical delivery. The appropriate route may be determined by one of skill in the art, as appropriate to the condition of the subject under treatment.

Vascular or extravascular circulation, the blood or lymph system, and cerebrospinal fluid are some non-limiting sites where the RNA may be introduced. Direct CNS delivery may be employed, for instance, intracerebral ventricular or intrathecal administration may be used as routes of administration.

In certain embodiments, the antisense oligomers, or pharmaceutically acceptable salts thereof, are administered to the subject by intramuscular injection (IM), i.e., they are administered or delivered intramuscularly. Non-limiting examples of intramuscular injection sites include the deltoid muscle of the arm, the vastus lateralis muscle of the leg, and the ventrogluteal muscles of the hips, and dorsogluteal muscles of the buttocks. In specific embodiments, a PMO, PMO-X, or PPMO is administered by IM.

In certain embodiments, the antisense oligomers, or pharmaceutically acceptable salts thereof, of the disclosure can be delivered by transdermal methods (e.g., via incorporation of the antisense oligomers, or pharmaceutically acceptable salts thereof, into, e.g., emulsions, with such antisense oligomers, or pharmaceutically acceptable salts thereof, optionally packaged into liposomes). Such transdermal and emulsion/liposome-mediated methods of delivery are described for delivery of antisense oligomers, or pharmaceutically acceptable salts thereof, in the art, e.g., in U.S. Pat. No. 6,965,025, the contents of which are incorporated herein by reference in their entireties.

The antisense oligomers, or pharmaceutically acceptable salts thereof, described herein may also be delivered via an implantable device. Design of such a device is an art-recognized process, with, e.g., synthetic implant design described in, e.g., U.S. Pat. No. 6,969,400, the contents of which are incorporated herein by reference in its entirety.

Antisense oligomers, or pharmaceutically acceptable salts thereof, can be introduced into cells using art-recognized techniques (e.g., transfection, electroporation, fusion, liposomes, colloidal polymeric particles, and viral and non-viral vectors as well as other means known in the art). The method of delivery selected will depend at least on the oligomer chemistry, the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, antisense oligomers, or pharmaceutically acceptable salts thereof, may be delivered using, e.g., methods involving liposome-mediated uptake, exosome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated herein by reference in its entirety).

The antisense oligomers, or pharmaceutically acceptable salts thereof, may be administered in any convenient vehicle or carrier which is physiologically and/or pharmaceutically acceptable. Such a composition may include any of a variety of standard pharmaceutically acceptable carriers employed by those of ordinary skill in the art. Examples include, but are not limited to, saline, phosphate-buffered saline (PBS), water, aqueous ethanol, emulsions, such as oil/water emulsions or triglyceride emulsions, tablets, and capsules. The choice of a suitable physiologically acceptable carrier will vary dependent upon the chosen mode of administration. “Pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The compounds (e.g., antisense oligomers, or pharmaceutically acceptable salts thereof) of the present disclosure may generally be utilized as the free acid or free base. Alternatively, the compounds of this disclosure may be used in the form of acid or base addition salts. Acid addition salts of the free amino compounds of the present disclosure may be prepared by methods well known in the art and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids.

Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” is intended to encompass any and all acceptable salt forms.

In addition, prodrugs are also included within the context of this disclosure. Prodrugs are any covalently bonded carriers that release a compound in vivo when such prodrug is administered to a patient. Prodrugs are generally prepared by modifying functional groups in a way such that the modification is cleaved, either by routine manipulation or in vivo, yielding the parent compound. Prodrugs include, for example, antisense oligomers, or pharmaceutically acceptable salts thereof, of this disclosure wherein hydroxy, amine, or sulfhydryl groups are bonded to any group that, when administered to a patient, cleaves to form the hydroxy, amine, or sulfhydryl groups. Thus, representative examples of prodrugs include (but are not limited to) acetate, formate, and benzoate derivatives of alcohol and amine functional groups of the antisense oligomers, or pharmaceutically acceptable salts thereof, of the disclosure. Further, in the case of a carboxylic acid (—COOH), esters may be employed, such as methyl esters, ethyl esters, and the like.

In some instances, liposomes may be employed to facilitate uptake of the antisense oligomer, or of a pharmaceutically acceptable salts thereof, into cells (see, e.g., Williams, S. A., Leukemia 10 (12): 1980-1989, 1996; Lappalainen et al., Antiviral Res. 23:119, 1994; Uhlmann et al., antisense oligomers: a new therapeutic principle, Chemical Reviews, Volume 90, No. 4, 25 pages 544-584, 1990; Gregoriadis, G., Chapter 14, Liposomes, Drug Carriers in Biology and Medicine, pp. 287-341, Academic Press, 1979). Hydrogels may also be used as vehicles for antisense oligomer, or pharmaceutically acceptable salt thereof, administration, for example, as described in WO 93/01286. Alternatively, the oligomers may be administered in microspheres or microparticles. (See, e.g., Wu, G. Y. and Wu, C. H., J. Biol. Chem. 262:4429-4432, 30 1987). Alternatively, the use of gas-filled microbubbles complexed with the antisense oligomers, or pharmaceutically acceptable salts thereof, can enhance delivery to target tissues, as described in U.S. Pat. No. 6,245,747. Sustained-release compositions may also be used. These may include semipermeable polymeric matrices in the form of shaped articles such as films or microcapsules.

In one embodiment, the antisense oligomer, or a pharmaceutically acceptable salt thereof, is administered to a mammalian subject, e.g., human or domestic animal, exhibiting the symptoms of a kidney disease, in a suitable pharmaceutical carrier. In one aspect of the method, the subject is a human subject, e.g., a patient diagnosed as having a chronic kidney disease such as ADTKD-UMOD. In one embodiment, the antisense oligomer, or a pharmaceutically acceptable salt thereof, is contained in a pharmaceutically acceptable carrier and is delivered orally. In another embodiment, the oligomer is contained in a pharmaceutically acceptable carrier and is delivered intravenously (i.v.).

In one embodiment, the antisense oligomer, or a pharmaceutically acceptable salt thereof, is administered in an amount and manner effective to result in a peak blood concentration of at least 200-400 nM antisense oligomer, or pharmaceutically acceptable salt thereof. Typically, one or more doses of antisense oligomer, or of a pharmaceutically acceptable salt thereof, are administered, generally at regular intervals, for a period of about one to two weeks. Doses for oral administration are from about 1-1000 mg oligomer per 70 kg. In some cases, doses of greater than 1000 mg oligomer/patient may be necessary. For i.v. administration, doses are from about 0.5 mg to 1000 mg oligomer per 70 kg. The antisense oligomer, or pharmaceutically acceptable salt thereof, may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests, and physiological examination of the subject under treatment.

In certain embodiments, the method is an in vitro method. In certain other embodiments, the method is an in vivo method.

In certain embodiments, the host cell is a mammalian cell. In certain embodiments, the host cell is a non-human primate cell. In certain embodiments, the host cell is a human cell.

In certain embodiments, the host cell is a naturally occurring cell. In certain other embodiments, the host cell is an engineered cell.

In certain embodiments, the antisense oligomer, or a pharmaceutically acceptable salt thereof, is administered to a mammalian subject, e.g., a human or a laboratory or domestic animal, in a suitable pharmaceutical carrier.

In certain embodiments, the antisense oligomer, or a pharmaceutically acceptable salt thereof, is administered to a mammalian subject, e.g., a human or laboratory or domestic animal, together with an additional agent. The antisense oligomer, or a pharmaceutically acceptable salt thereof, and the additional agent can be administered simultaneously or sequentially, via the same or different routes and/or sites of administration. In certain embodiments, the antisense oligomer, or a pharmaceutically acceptable salt thereof, and the additional agent can be co-formulated and administered together. In certain embodiments, the antisense oligomer, or a pharmaceutically acceptable salt thereof, and the additional agent can be provided together in a kit.

In one embodiment, the antisense oligomer, or a pharmaceutically acceptable salt thereof, contained in a pharmaceutically acceptable carrier, is delivered orally.

In one embodiment, the antisense oligomer, or a pharmaceutically acceptable salt thereof, contained in a pharmaceutically acceptable carrier, is delivered intravenously (i.v.).

Additional routes of administration, e.g., subcutaneous, intraperitoneal, and pulmonary, are also contemplated by the instant disclosure.

In another application of the method, the subject is a livestock animal, e.g., a pig, cow, or goat, etc., and the treatment is either prophylactic or therapeutic. Also contemplated is, in a method of feeding livestock with a food substance, an improvement in which the food substance is supplemented with an effective amount of an antisense oligomer, or of a pharmaceutically acceptable salt thereof, composition as described above.

In an embodiment, the antisense oligomer, or a pharmaceutically acceptable salt thereof, is administered in an amount and manner effective to result in a peak blood concentration of at least 200 nM antisense oligomer, or pharmaceutically acceptable salt thereof. In one embodiment, the antisense oligomer, or a pharmaceutically acceptable salt thereof, is administered in an amount and manner effective to result in a peak plasma concentration of at least 200 nM antisense oligomer, or pharmaceutically acceptable salt thereof. In one embodiment, the antisense oligomer, or a pharmaceutically acceptable salt thereof, is administered in an amount and manner effective to result in a peak serum concentration of at least 200 nM antisense oligomer, or pharmaceutically acceptable salt thereof.

In an embodiment, the antisense oligomer, or a pharmaceutically acceptable salt thereof, is administered in an amount and manner effective to result in a peak blood concentration of at least 400 nM antisense oligomer, or pharmaceutically acceptable salt thereof. In one embodiment, the antisense oligomer, or a pharmaceutically acceptable salt thereof, is administered in an amount and manner effective to result in a peak plasma concentration of at least 400 nM antisense oligomer, or pharmaceutically acceptable salt thereof. In one embodiment, the antisense oligomer, or a pharmaceutically acceptable salt thereof, is administered in an amount and manner effective to result in a peak serum concentration of at least 400 nM antisense oligomer, or pharmaceutically acceptable salt thereof.

Typically, one or more doses of the antisense oligomer, or a pharmaceutically acceptable salt thereof, are administered, generally at regular intervals, for a period of about one to two weeks. Doses for oral administration are from about 0.01-15 mg antisense oligomer, or pharmaceutically acceptable salt thereof, per kg body weight. In some cases, doses of greater than 15 mg antisense oligomer (or pharmaceutically acceptable salt thereof)/kg may be necessary. For i.v. administration, doses are from about 0.005 mg to 15 mg antisense oligomer (or pharmaceutically acceptable salt thereof) per kg body weight. The antisense oligomer, or a pharmaceutically acceptable salt thereof, may be administered at regular intervals for a short time period, e.g., daily for two weeks or less. However, in some cases, the antisense oligomer, or a pharmaceutically acceptable salt thereof, is administered intermittently over a longer period of time. Administration may be followed by or accompanied by administration of an antibiotic or other therapeutic treatment. The treatment regimen may be adjusted (dose, frequency, route, etc.) as indicated, based on the results of immunoassays, other biochemical tests, and physiological examination of the subject under treatment.

An effective in vivo treatment regimen using the antisense oligomer, or a pharmaceutically acceptable salt thereof, may vary according to the duration, dose, frequency, and route of administration, as well as the condition of the subject under treatment (i.e., prophylactic administration versus administration in response to localized or systemic infection). Accordingly, such in vivo therapy will often require monitoring by tests under treatment, and corresponding adjustments in the dose or treatment regimen, in order to achieve an optimal therapeutic outcome.

In some embodiments, the antisense oligomer, or a pharmaceutically acceptable salt thereof, is actively taken up by mammalian cells. In further embodiments, the antisense oligomer, or a pharmaceutically acceptable salt thereof, can be conjugated to a transport moiety (e.g., transport peptide) as described herein to facilitate such uptake.

### Incorporation by Reference

All publications, patents, and patent applications mentioned in this specification are incorporated herein by reference to the same extent as if each individual publication, patent, or patent application was specifically and individually indicated to be incorporated herein by reference.

### IX. Additional Embodiments

Additional embodiments include embodiments 1 to 199 following.

Embodiment 1. An antisense oligomer, or a pharmaceutically acceptable salt thereof, comprising a non-natural chemical backbone and a targeting sequence of 13 to 30 bases in length that is complementary to a target region within a pre-mRNA of the human uromodulin (UMOD) gene represented by SEQ ID NO: 1, wherein the target region is an intron/exon junction or an exon internal region of the human UMOD gene pre-mRNA.

Embodiment 2. The antisense oligomer, or a pharmaceutically acceptable salt thereof, of embodiment 1, wherein the target region is an intron/exon junction or exon internal sequence of exon 2 (SEQ ID NO: 2), exon 5 (SEQ ID NO: 3), exon 6 (SEQ ID NO: 4), exon 8 (SEQ ID NO: 5), or exon 9 (SEQ ID NO: 6).

Embodiment 3. The antisense oligomer, or a pharmaceutically acceptable salt thereof, of embodiment 1 or 2, wherein the target region is an intron/exon junction or exon internal region of exon 2 (SEQ ID NO: 2).

Embodiment 4. The antisense oligomer, or a pharmaceutically acceptable salt thereof, of any one of embodiments 1-3, wherein the target region is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175).

Embodiment 5. The antisense oligomer, or a pharmaceutically acceptable salt thereof, of any one of embodiments 1-4, wherein the targeting sequence comprises a sequence selected from SEQ ID NOs: 11-17, 20, 23, and 26-28.

Embodiment 6. The antisense oligomer, or a pharmaceutically acceptable salt thereof, of any one of embodiments 1-3, wherein the target region is within the 51^{st} nucleotide to the 119^{th} nucleotide from the 5′ end of exon 2 (SEQ ID NO: 2) of the human UMOD gene pre-mRNA.

Embodiment 7. The antisense oligomer, or a pharmaceutically acceptable salt thereof, of any one of embodiments 1-3 or 6, wherein the target region is within the 51^{st} nucleotide to the 105^{th} nucleotide from the 5′ end of exon 2 (SEQ ID NO: 2) of the human UMOD gene pre-mRNA.

Embodiment 8. The antisense oligomer, or a pharmaceutically acceptable salt thereof, of any one of embodiments 1-3, 6, or 7, wherein the target region is H2A(+51+75).

Embodiment 9. The antisense oligomer, or a pharmaceutically acceptable salt thereof, of any one of embodiments 1-3 or 6-8, wherein the targeting sequence comprises SEQ ID NO: 14.

Embodiment 10. The antisense oligomer, or a pharmaceutically acceptable salt thereof, of any one of embodiments 1-3, wherein the target region is within the 85^{th} nucleotide to the 119^{th} nucleotide from the 5′ end of exon 2 (SEQ ID NO: 2) of the human UMOD gene pre-mRNA.

Embodiment 11. The antisense oligomer, or a pharmaceutically acceptable salt thereof, of any one of embodiments 1-3 or 10, wherein the target region is selected from H2A(+85+104), H2A(+86+105), and H2A(+95+119).

Embodiment 12. The antisense oligomer, or a pharmaceutically acceptable salt thereof, of any one of embodiments 1-3, 10, or 11, wherein the targeting sequence comprises a sequence selected from SEQ ID NOs: 15-17.

Embodiment 13. The antisense oligomer, or a pharmaceutically acceptable salt thereof, of any one of embodiments 1-3, wherein the target region is within the 15^{th} nucleotide of intron 1 measured from the 5′ end of exon 2 (SEQ ID NO: 2) to the 25^{th} nucleotide of exon 2 measured from the 5′ end of exon 2 (SEQ ID NO: 2).

Embodiment 14. The antisense oligomer, or a pharmaceutically acceptable salt thereof, of any one of embodiments 1-3 or 13, wherein the target region is selected from H2A(−15+10) and H2A(+1+25).

Embodiment 15. The antisense oligomer, or a pharmaceutically acceptable salt thereof, of any one of embodiments 1-3 or 13-14, wherein the targeting sequence comprises SEQ ID NO: 12 or 13.

Embodiment 16. The antisense oligomer, or a pharmaceutically acceptable salt thereof, of any one of embodiments 1-3, wherein the target region is within the 118^{th} nucleotide to the 150^{th} nucleotide of exon 2 as measured from the 5′ end of exon 2 (SEQ ID NO: 2).

Embodiment 17. The antisense oligomer, or a pharmaceutically acceptable salt thereof, of any one of embodiments 1-3 or 16, wherein the target region is selected from H2A(+118+137) and H2A(+126+150).

Embodiment 18. The antisense oligomer, or a pharmaceutically acceptable salt thereof, of any one of embodiments 1-3 or 16-17, wherein the targeting sequence comprises a sequence selected from SEQ ID NOs: 26 and 27.

Embodiment 19. The antisense oligomer, or a pharmaceutically acceptable salt thereof, of any one of embodiments 1-3, wherein the target region is selected from H2A(+101+125), H2A(+110+129), and H2A(+151+175).

Embodiment 20. The antisense oligomer, or a pharmaceutically acceptable salt thereof, of any one of embodiments 1-3 or 19, wherein the targeting sequence comprises a sequence selected from SEQ ID NOs: 20, 21, and 28.

Embodiment 21. The antisense oligomer, or a pharmaceutically acceptable salt thereof, of embodiment 1 or 2, wherein the target region is an intron/exon junction or exon internal region of exon 5 (SEQ ID NO: 3).

Embodiment 22. The antisense oligomer, or a pharmaceutically acceptable salt thereof, of any one of embodiments 1-2 or 21, wherein the target region is selected from H5A(−15+5), H5A(−14+6), H5A(−11+9), H5A(−10+10), H5A(+51+75), H5A(+76+100), H5A(+78+95), H5A(+80+97), H5A(+82+99), H5A(+126+150), H5A(+153+172), H5A(+154+173), H5D(+18-2), H5D(+16-4), H5D(+14-6), H5D(+13-7), and H5D(+12-8).

Embodiment 23. The antisense oligomer, or a pharmaceutically acceptable salt thereof, of any one of embodiments 1-2 or 21-22, wherein the targeting sequence comprises a sequence selected from SEQ ID NOs: 30-33, 35, 37-40, 44, 46-48, and 50-53.

Embodiment 24. The antisense oligomer, or a pharmaceutically acceptable salt thereof, of any one of embodiments 1-2 or 21, wherein the target region is within the 15^{th} nucleotide of intron 4 measured from the 5′ end of exon 5 (SEQ ID NO: 3) to the 10^{th} nucleotide of exon 5 measured from the 5′ end of exon 5 (SEQ ID NO: 3) of the human UMOD gene pre-mRNA.

Embodiment 25. The antisense oligomer, or a pharmaceutically acceptable salt thereof, of any one of embodiments 1-2, 21-22, or 24, wherein the target region is selected from H5A(−15+5), H5A(−14+6), H5A(−11+9), and H5A(−10+10).

Embodiment 26. The antisense oligomer, or a pharmaceutically acceptable salt thereof, of any one of embodiments 1-2, 21-22, or 24-25, wherein the target region is H5A(−15+5).

Embodiment 27. The antisense oligomer, or a pharmaceutically acceptable salt thereof, of any one of embodiments 1-2, 21-22, or 24-26, wherein the targeting sequence comprises SEQ ID NO: 30.

Embodiment 28. The antisense oligomer, or a pharmaceutically acceptable salt thereof, of any one of embodiments 1-2, 21-22, or 24-25, wherein the target region is H5A(−14+6).

Embodiment 29. The antisense oligomer, or a pharmaceutically acceptable salt thereof, of 1-2, 21-22, 24-25, or 29, wherein the targeting sequence comprises SEQ ID NO: 31.

Embodiment 30. The antisense oligomer, or a pharmaceutically acceptable salt thereof, of any one of embodiments 1-2, 21-22, or 24-25, wherein the target region is H5A(−11+9).

Embodiment 31. The antisense oligomer, or a pharmaceutically acceptable salt thereof, of any one of embodiments 1-2, 21-22, 24-25, or 30, wherein the targeting sequence comprises SEQ ID NO: 32.

Embodiment 32. The antisense oligomer, or a pharmaceutically acceptable salt thereof, of any one of embodiments 1-2, 21-22, or 24-25, wherein the target region is H5A(−10+10).

Embodiment 33. The antisense oligomer, or a pharmaceutically acceptable salt thereof, of any one of embodiments 1-2, 21-22, 24-25, or 32, wherein the targeting sequence comprises SEQ ID NO: 33.

Embodiment 34. The antisense oligomer, or a pharmaceutically acceptable salt thereof, of any one of embodiments 1-2 or 21-22, wherein the target region is H5A(+51+75).

Embodiment 35. The antisense oligomer, or a pharmaceutically acceptable salt thereof, of any one of embodiments 1-2, 21-22, or 34, wherein the targeting sequence comprises SEQ ID NO:

35.

Embodiment 36. The antisense oligomer, or a pharmaceutically acceptable salt thereof, of any one of embodiments 1-2 or 21, wherein the target region is within the 76^{th} nucleotide to the 100^{th} nucleotide measured from the 5′ end of exon 5 (SEQ ID NO: 3) of the human UMOD gene pre-mRNA.

Embodiment 37. The antisense oligomer, or a pharmaceutically acceptable salt thereof, of any one of embodiments 1-2, 21-22, or 36, wherein the target region is selected from H5A(+76+100), H5A(+78+95), H5A(+80+97), and H5A(+82+99).

Embodiment 38. The antisense oligomer, or a pharmaceutically acceptable salt thereof, of any one of embodiments 1-2, 21-22, or 36-37, wherein the target region is H5A(+76+100).

Embodiment 39. The antisense oligomer, or a pharmaceutically acceptable salt thereof, of any one of embodiments 1-2, 21-22, or 36-38, wherein the targeting sequence comprises SEQ ID NO: 37.

Embodiment 40. The antisense oligomer, or a pharmaceutically acceptable salt thereof, of any one of embodiments 1-2, 21-22, or 36-37, wherein the target region is H5A(+78+95).

Embodiment 41. The antisense oligomer, or a pharmaceutically acceptable salt thereof, of any one of embodiments 1-2, 21-22, 36-37, or 40, wherein the targeting sequence comprises SEQ ID NO: 38.

Embodiment 42. The antisense oligomer, or a pharmaceutically acceptable salt thereof, of any one of embodiments 1-2, 21-22, or 36-37, wherein the target region is H5A(+80+97).

Embodiment 43. The antisense oligomer, or a pharmaceutically acceptable salt thereof, of any one of embodiments 1-2, 21-22, 36-37, or 42, wherein the targeting sequence comprises SEQ ID NO: 39.

Embodiment 44. The antisense oligomer, or a pharmaceutically acceptable salt thereof, of any one of embodiments 1-2, 21-22, or 36-37, wherein the target region is H5A(+82+99).

Embodiment 45. The antisense oligomer, or a pharmaceutically acceptable salt thereof, of any one of embodiments 1-2, 21-22, 36-37, or 44, wherein the targeting sequence comprises SEQ ID NO: 40.

Embodiment 46. The antisense oligomer, or a pharmaceutically acceptable salt thereof, of any one of embodiments 1-2 or 21-22, wherein the target region is H5A(+126+150).

Embodiment 47. The antisense oligomer, or a pharmaceutically acceptable salt thereof, of any one of embodiments 1-2, 21-22, or 46, wherein the targeting sequence comprises SEQ ID NO: 44.

Embodiment 48. The antisense oligomer, or a pharmaceutically acceptable salt thereof, of any one of embodiments 1-2 or 21, wherein the target region is within the 153^{rd} nucleotide to the 173^{rd} nucleotide measured from the 5′ end of exon 5 (SEQ ID NO: 3) of the human UMOD gene pre-mRNA.

Embodiment 49. The antisense oligomer, or a pharmaceutically acceptable salt thereof, of any one of embodiments 1-2, 21-22, or 48, wherein the target region is selected from H5A(+153+172) and H5A(+154+173).

Embodiment 50. The antisense oligomer, or a pharmaceutically acceptable salt thereof, of any one of embodiments 1-2, 21-22, or 48-49, wherein the targeting sequence comprises a sequence selected from SEQ ID NOs: 46 and 47.

Embodiment 51. The antisense oligomer, or a pharmaceutically acceptable salt thereof, of any one of embodiments 1-2 or 21, wherein the target region is within the 18^{th} nucleotide of exon 5 measured from 3′ end of exon 5 (SEQ ID NO: 3) to the 8^{th} nucleotide of intron 5 measured from the 3′ end of exon 5 (SEQ ID NO: 3).

Embodiment 52. The antisense oligomer, or a pharmaceutically acceptable salt thereof, of any one of embodiments 1-2, 21-22, or 51, wherein the target region is selected from H5D(+18-2), H5D(+16-4), H5D(+14-6), H5D(+13-7), and H5D(+12-8).

Embodiment 53. The antisense oligomer, or a pharmaceutically acceptable salt thereof, of any one of embodiments 1-2, 21-22, or 51-52, wherein the targeting sequence comprises a sequence selected from SEQ ID NOs: 48 and 50-53.

Embodiment 54. The antisense oligomer, or a pharmaceutically acceptable salt thereof, of embodiment 1 or 2, wherein the target region is an intron/exon junction or exon internal region of exon 6 (SEQ ID NO: 4).

Embodiment 55. The antisense oligomer, or a pharmaceutically acceptable salt thereof, of any one of embodiments 1-2 or 54, wherein the target region is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D(+24-1), H6D(+15-5), and H6D(+14-6).

Embodiment 56. The antisense oligomer, or a pharmaceutically acceptable salt thereof, of any one of embodiments 1-2 or 54-55, wherein the targeting sequence comprises a sequence selected from SEQ ID NOs: 57-59, 61, 62, 64, and 66-81.

Embodiment 57. The antisense oligomer, or a pharmaceutically acceptable salt thereof, of any one of embodiments 1-2 or 54-55, wherein the target region is H6A(+26+50).

Embodiment 58. The antisense oligomer, or a pharmaceutically acceptable salt thereof, of any one of embodiments 1-2 or 54-57, wherein the targeting sequence comprises SEQ ID NO: 57.

Embodiment 59. The antisense oligomer, or a pharmaceutically acceptable salt thereof, of any one of embodiments 1-2 or 54, wherein the target region is within the 48^{th} nucleotide to the 78^{th} nucleotide measured from the 5′ end of exon 6 (SEQ ID NO: 4) of the human UMOD gene pre-mRNA.

Embodiment 60. The antisense oligomer, or a pharmaceutically acceptable salt thereof, of any one of embodiments 1-2, 54-55, or 59, wherein the target region is selected from H6A(+48+67), H6A(+49+68), H6A(+58+77), and H6A(+59+78).

Embodiment 61. The antisense oligomer, or a pharmaceutically acceptable salt thereof, of any one of embodiments 1-2, 54-55, or 59-60, wherein the targeting sequence comprises a sequence selected from SEQ ID NOs: 58, 59, 61, and 62.

Embodiment 62. The antisense oligomer, or a pharmaceutically acceptable salt thereof, of any one of embodiments 1-2 or 54, wherein the target region is within the 58^{th} nucleotide to the 78^{th} nucleotide measured from the 5′ end of exon 6 (SEQ ID NO: 4) of the human UMOD gene pre-mRNA.

Embodiment 63. The antisense oligomer, or a pharmaceutically acceptable salt thereof, of any one of embodiments 1-2, 54-55, or 62, wherein the target region is selected from H6A(+58+77) and H6A(+59+78).

Embodiment 64. The antisense oligomer, or a pharmaceutically acceptable salt thereof, of any one of embodiments 1-2, 54-55, or 62-63, wherein the target region is H6A(+58+77).

Embodiment 65. The antisense oligomer, or a pharmaceutically acceptable salt thereof, of any one of embodiments 1-2, 54-55, or 62-64, wherein the targeting sequence comprises SEQ ID NO: 61.

Embodiment 66. The antisense oligomer, or a pharmaceutically acceptable salt thereof, of any one of embodiments 1-2, 54-55, or 62-63, wherein the target region is H6A(+59+78).

Embodiment 67. The antisense oligomer, or a pharmaceutically acceptable salt thereof, of any one of embodiments 1-2, 54, 62-63, or 66, wherein the targeting sequence comprises SEQ ID NO: 62.

Embodiment 68. The antisense oligomer, or a pharmaceutically acceptable salt thereof, of any one of embodiments 1-2 or 54-55, wherein the target region is H6A(+76+100).

Embodiment 69. The antisense oligomer, or a pharmaceutically acceptable salt thereof, of any one of embodiments 1-2, 54-55, or 68, wherein the targeting sequence comprises SEQ ID NO: 64.

Embodiment 70. The antisense oligomer, or a pharmaceutically acceptable salt thereof, of any one of embodiments 1-2 or 54, wherein the target region is within the 101^{st} nucleotide to the 132^{nd} nucleotide measured from the 5′ end of exon 6 (SEQ ID NO: 4) of the human UMOD gene pre-mRNA.

Embodiment 71. The antisense oligomer, or a pharmaceutically acceptable salt thereof, of any one of embodiments 1-2, 54-55, or 70, wherein the target region is selected from H6A(+101+125), H6A(+101+120), H6A(+110+129), H6A(+111+130), H6A(+112+131), and H6A(+113+132).

Embodiment 72. The antisense oligomer, or a pharmaceutically acceptable salt thereof, of any one of embodiments 1-2, 54-55, or 70-71, wherein the targeting sequence comprises a sequence selected from SEQ ID Nos: 66-71.

Embodiment 73. The antisense oligomer, or a pharmaceutically acceptable salt thereof, of any one of embodiments 1-2 or 54, wherein the target region is within the 111^{th} nucleotide to the 132^{nd} nucleotide measured from the 5′ end of exon 6 (SEQ ID NO: 4) of the human UMOD gene pre-mRNA.

Embodiment 74. The antisense oligomer, or a pharmaceutically acceptable salt thereof, of any one of embodiments 1-2, 54-55, or 73, wherein the target region is selected from H6A(+111+130), H6A(+112+131), and H6A(+113+132).

Embodiment 75. The antisense oligomer, or a pharmaceutically acceptable salt thereof, of any one of embodiments 1-2, 54-55, or 73-74, wherein the target region is H6A(+111+130).

Embodiment 76. The antisense oligomer, or a pharmaceutically acceptable salt thereof, of any one of embodiments 1-2, 54-55, or 73-75, wherein the targeting sequence comprises SEQ ID NO: 69.

Embodiment 77. The antisense oligomer, or a pharmaceutically acceptable salt thereof, of any one of embodiments 1-2, 54-55, or 73-74, wherein the target region is H6A(+112+131).

Embodiment 78. The antisense oligomer, or a pharmaceutically acceptable salt thereof, of any one of embodiments 1-2, 54-55, 73-74, or 77, wherein the targeting sequence comprises SEQ ID NO: 70.

Embodiment 79. The antisense oligomer, or a pharmaceutically acceptable salt thereof, of any one of embodiments 1-2, 54-55, or 73-74, wherein the target region is H6A(+113+132).

Embodiment 80. The antisense oligomer, or a pharmaceutically acceptable salt thereof, of any one of embodiments 1-2, 54-55, 73-74, or 79, wherein the targeting sequence comprises SEQ ID NO: 71.

Embodiment 81. The antisense oligomer, or a pharmaceutically acceptable salt thereof, of any one of embodiments 1-2 or 54, wherein the target region is within the 119^{th} nucleotide to the 149^{th} nucleotide measured from the 5′ end of exon 6 (SEQ ID NO: 4) of the human UMOD gene pre-mRNA.

Embodiment 82. The antisense oligomer, or a pharmaceutically acceptable salt thereof, of any one of embodiments 1-2, 54-55, or 81, wherein the target region is selected from H6A(+119+138), H6A(+120+139), H6A(+121+140), H6A(+122+141), H6A(+123+142), H6A(+124+143), and H6A(+130+149).

Embodiment 83. The antisense oligomer, or a pharmaceutically acceptable salt thereof, of any one of embodiments 1-2, 54-55, or 81-82, wherein the targeting sequence comprises a sequence selected from SEQ ID Nos: 72-78.

Embodiment 84. The antisense oligomer, or a pharmaceutically acceptable salt thereof, of any one of embodiments 1-2 or 54, wherein the target region is within the 119^{th} nucleotide to the 141^{st} nucleotide measured from the 5′ end of exon 6 (SEQ ID NO: 4) of the human UMOD gene pre-mRNA.

Embodiment 85. The antisense oligomer, or a pharmaceutically acceptable salt thereof, of any one of embodiments 1-2, 54-55, or 84, wherein the target region is selected from H6A(+119+138), H6A(+120+139), H6A(+121+140), H6A(+122+141).

Embodiment 86. The antisense oligomer, or a pharmaceutically acceptable salt thereof, of any one of embodiments 1-2, 54-55, or 84-85, wherein the targeting sequence comprises a sequence selected from SEQ ID NOs: 72-75.

Embodiment 87. The antisense oligomer, or a pharmaceutically acceptable salt thereof, of any one of embodiments 1-2 or 54, wherein the target region is within the 120^{th} nucleotide to the 141^{st} nucleotide measured from the 5′ end of exon 6 (SEQ ID NO: 4) of the human UMOD gene pre-mRNA.

Embodiment 88. The antisense oligomer, or a pharmaceutically acceptable salt thereof, of any one of embodiments 1-2, 54-55, or 87, wherein the target region is selected from H6A(+120+139), H6A(+121+140), and H6A(+122+141).

Embodiment 89. The antisense oligomer, or a pharmaceutically acceptable salt thereof, of any one of embodiments 1-2, 54-55, or 87-88, wherein the target region is H6A(+120+139).

Embodiment 90. The antisense oligomer, or a pharmaceutically acceptable salt thereof, of 1-2, 54-55, or 87-89, wherein the targeting sequence comprises SEQ ID NO: 73.

Embodiment 91. The antisense oligomer, or a pharmaceutically acceptable salt thereof, of any one of embodiments 1-2, 54-55, or 87-88, wherein the target region is H6A(+121+140).

Embodiment 92. The antisense oligomer, or a pharmaceutically acceptable salt thereof, of any one of embodiments 1-2, 54-55, 87-88, or 91, wherein the targeting sequence comprises SEQ ID NO: 74.

Embodiment 93. The antisense oligomer, or a pharmaceutically acceptable salt thereof, of any one of embodiments 1-2, 54-55, or 87-88, wherein the target region is H6A(+122+141).

Embodiment 94. The antisense oligomer, or a pharmaceutically acceptable salt thereof, of any one of embodiments 1-2, 54-55, 87-88, or 93, wherein the targeting sequence comprises SEQ ID NO: 75.

Embodiment 95. The antisense oligomer, or a pharmaceutically acceptable salt thereof, of any one of embodiments 1-2 or 54, wherein the target region is within the 123^{rd} nucleotide to the 149^{th} nucleotide measured from the 5′ end of exon 6 (SEQ ID NO: 4) of the human UMOD gene pre-mRNA.

Embodiment 96. The antisense oligomer, or a pharmaceutically acceptable salt thereof, of any one of embodiments 1-2, 54-55, or 95, wherein the target region is selected from H6A(+123+142), H6A(+124+143), and H6A(+130+149).

Embodiment 97. The antisense oligomer, or a pharmaceutically acceptable salt thereof, of any one of embodiments 1-2, 54-55, or 95-96, wherein the targeting sequence comprises a sequence selected from SEQ ID NOs: 76-78.

Embodiment 98. The antisense oligomer, or a pharmaceutically acceptable salt thereof, of any one of embodiments 1-2 or 54, wherein the target is within the 24^{th} nucleotide of exon 6 measured from 3′ end of exon 6 (SEQ ID NO: 4) to the 6^{th} nucleotide of intron 6 measured from the 3′ end of exon 6 (SEQ ID NO: 4) of the human UMOD gene pre-mRNA.

Embodiment 99. The antisense oligomer, or a pharmaceutically acceptable salt thereof, of any one of embodiments 1-2, 54-55, or 98, wherein the target region is selected from H6D(+24-1), H6D(+15-5), and H6D(+14-6).

Embodiment 100. The antisense oligomer, or a pharmaceutically acceptable salt thereof, of any one of embodiments 1-2, 54-55, or 98-99, wherein the targeting sequence comprises a sequence selected from SEQ ID NOs: 79-81.

Embodiment 101. The antisense oligomer, or a pharmaceutically acceptable salt thereof, of any one of embodiments 1-2 or 54, wherein the target region is within the 15^{th} nucleotide of exon 6 measured from 3′ end of exon 6 (SEQ ID NO: 4) to the 6^{th} nucleotide of intron 6 measured from the 3′ end of exon 6 (SEQ ID NO: 4) of the human UMOD gene pre-mRNA.

Embodiment 102. The antisense oligomer, or a pharmaceutically acceptable salt thereof, of any one of embodiments 1-2, 54-55, or 101, wherein the target region is selected from H6D(+15-5) and H6D(+14-6).

Embodiment 103. The antisense oligomer, or a pharmaceutically acceptable salt thereof, of any one of embodiments 1-2, 54-55, or 101-102, wherein the target region is H6A(+15-5).

Embodiment 104. The antisense oligomer, or a pharmaceutically acceptable salt thereof, of any one of embodiments 1-2, 54-55, or 101-103, wherein the targeting sequence comprises SEQ ID NO: 80.

Embodiment 105. The antisense oligomer, or a pharmaceutically acceptable salt thereof, of any one of embodiments 1-2, 54-55, or 101-102, wherein the target region is H6A(+14-6).

Embodiment 106. The antisense oligomer, or a pharmaceutically acceptable salt thereof, of any one of embodiments 1-2, 54, 101-102, or 105, wherein the targeting sequence comprises SEQ ID NO: 81.

Embodiment 107. The antisense oligomer, or a pharmaceutically acceptable salt thereof, of embodiment 1 or 2, wherein the target region is an intron/exon junction or exon internal region of exon 8 (SEQ ID NO: 5).

Embodiment 108. The antisense oligomer, or a pharmaceutically acceptable salt thereof, of any one of embodiments 1-2 or 107, wherein the target region is selected from H8A(−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D(+12-13).

Embodiment 109. The antisense oligomer, or a pharmaceutically acceptable salt thereof, of any one of embodiments 1-2 or 107-108, wherein the targeting sequence comprises a sequence selected from SEQ ID NOs: 82, 84-86, 89-96, 98-115, and 120.

Embodiment 110. The antisense oligomer, or a pharmaceutically acceptable salt thereof, of any one of embodiments 1-2 or 107-108, wherein the target region is H8A(−2+23).

Embodiment 111. The antisense oligomer, or a pharmaceutically acceptable salt thereof, of any one of embodiments 1-2 or 107-109, wherein the targeting sequence comprises SEQ ID NO: 82.

Embodiment 112. The antisense oligomer, or a pharmaceutically acceptable salt thereof, of any one of embodiments 1-2 or 107, wherein the target region is within the 51^{st} and the 80^{th} nucleotide measured from the 5′ end of exon 8 (SEQ ID NO: 5) of the human UMOD gene pre-mRNA.

Embodiment 113. The antisense oligomer, or a pharmaceutically acceptable salt thereof, of any one of embodiments 1-2, 107-108, or 112, wherein the target region is selected from H8A(+51+75), H8A(+60+79), and H8A(+61+80).

Embodiment 114. The antisense oligomer, or a pharmaceutically acceptable salt thereof, of any one of embodiments 1-2, 107-108, or 112-113, wherein the targeting sequence comprises a sequence selected from SEQ ID NOs: 84-86.

Embodiment 115. The antisense oligomer, or a pharmaceutically acceptable salt thereof, of any one of embodiments 1-2 or 107, wherein the target region is within the 68^{th} nucleotide to the 100^{th} nucleotide measured from the 5′ end of exon 8 (SEQ ID NO:5) of the human UMOD gene pre-mRNA.

Embodiment 116. The antisense oligomer, or a pharmaceutically acceptable salt thereof, of any one of embodiments 1-2, 107-108, or 115, wherein the target region is selected from H8A(+68+87), H8A(+69+88), H8A(+70+89), H8A(+76+95), H8A(+76+100), H8A(+77+96), H8A(+78+97), and H8A(+79+98).

Embodiment 117. The antisense oligomer, or a pharmaceutically acceptable salt thereof, of any one of embodiments 1-2, 107-108, or 115-116, wherein the targeting sequence comprises a sequence selected from SEQ ID NOs: 89-96.

Embodiment 118. The antisense oligomer, or a pharmaceutically acceptable salt thereof, of any one of embodiments 1-2 or 107, wherein the target region is within the 76^{th} and the 100^{th} nucleotide measured from the 5′ end of exon 8 (SEQ ID NO:5) of the human UMOD gene pre-mRNA.

Embodiment 119. The antisense oligomer, or a pharmaceutically acceptable salt thereof, of any one of embodiments 1-2, 107-108, or 118, wherein the target region is selected from H8A(+76+95), H8A(+77+96), H8A(+78+97), and H8A(+79+98).

Embodiment 120. The antisense oligomer, or a pharmaceutically acceptable salt thereof, of any one of embodiments 1-2, 107-108, or 118-119, wherein the targeting sequence comprises a sequence selected from SEQ ID NOs: 92 and 94-96.

Embodiment 121. The antisense oligomer, or a pharmaceutically acceptable salt thereof, of any one of embodiments 1-2 or 107, wherein the target region is within the 81^{st} and the 105^{th} nucleotide measured from the 5′ end of exon 8 (SEQ ID NO:5) of the human UMOD gene pre-mRNA.

Embodiment 122. The antisense oligomer, or a pharmaceutically acceptable salt thereof, of any one of embodiments 1-2, 107-108, or 121, wherein the target region is selected from H8A(+81+100), H8A(+82+101), H8A(+83+102), and H8A(+86+105).

Embodiment 123. The antisense oligomer, or a pharmaceutically acceptable salt thereof, of any one of embodiments 1-2, 107-108, or 121-122, wherein the targeting sequence comprises a sequence selected from SEQ ID NOs: 98-101.

Embodiment 124. The antisense oligomer, or a pharmaceutically acceptable salt thereof, of any one of embodiments 1-2 or 107, wherein the target region is within the 94^{th} and the 124^{th} nucleotide measured from the 5′ end of exon 8 (SEQ ID NO:5) of the human UMOD gene pre-mRNA.

Embodiment 125. The antisense oligomer, or a pharmaceutically acceptable salt thereof, of any one of embodiments 1-2, 107-108, or 124, wherein the target region is selected from H8A(+94+113), H8A(+95+114), H8A(+96+115), H8A(+101+125), H8A(+102+121), H8A(+103+122), H8A(+104+123), and H8A(+105+124).

Embodiment 126. The antisense oligomer, or a pharmaceutically acceptable salt thereof, of any one of embodiments 1-2, 107-108, or 124-125, wherein the targeting sequence comprises a sequence selected from SEQ ID NOs: 102-109.

Embodiment 127. The antisense oligomer, or a pharmaceutically acceptable salt thereof, of any one of embodiments 1-2 or 107, wherein the target region is within the 120^{th} and the 148^{th} nucleotide measured from the 5′ end of exon 8 (SEQ ID NO:5) of the human UMOD gene pre-mRNA.

Embodiment 128. The antisense oligomer, or a pharmaceutically acceptable salt thereof, of any one of embodiments 1-2, 107-108, or 127, wherein the target region is selected from H8A(+120+139), H8A(+121+140), H8A(+122+141), H8A(+126+150), H8A(+128+147), and H8A(+129+148).

Embodiment 129. The antisense oligomer, or a pharmaceutically acceptable salt thereof, of any one of embodiments 1-2, 107-108, or 127-128, wherein the targeting sequence comprises a sequence selected from SEQ ID NOs: 110-115.

Embodiment 130. The antisense oligomer, or a pharmaceutically acceptable salt thereof, of any one of embodiments 1-2 or 107, wherein the target region is within the 126^{th} and the 148^{th} nucleotide measured from the 5′ end of exon 8 (SEQ ID NO:5) of the human UMOD gene pre-mRNA.

Embodiment 131. The antisense oligomer, or a pharmaceutically acceptable salt thereof, of any one of embodiments 1-2, 107-108, or 130, wherein the target region is selected from H8A(+126+150), H8A(+128+147), and H8A(+129+148).

Embodiment 132. The antisense oligomer, or a pharmaceutically acceptable salt thereof, of any one of embodiments 1-2, 107-108, or 130-131, wherein the target region is H8A(+126+150).

Embodiment 133. The antisense oligomer, or a pharmaceutically acceptable salt thereof, of any one of embodiments 1-2, 107-108, or 130-132, wherein the targeting sequence comprises SEQ ID NO: 113

Embodiment 134. The antisense oligomer, or a pharmaceutically acceptable salt thereof, of any one of embodiments 1-2, 107-108, or 130-131, wherein the target region is H8A(+128+147).

Embodiment 135. The antisense oligomer, or a pharmaceutically acceptable salt thereof, of any one of embodiments 1-2, 107-108, 130-131, or 134, wherein the targeting sequence comprises SEQ ID NO: 114.

Embodiment 136. The antisense oligomer, or a pharmaceutically acceptable salt thereof, of any one of embodiments 1-2, 107-108, or 130-131, wherein the target region is H8A(+129+148).

Embodiment 137. The antisense oligomer, or a pharmaceutically acceptable salt thereof, of any one of embodiments 1-2, 107-108, 130-131, or 136, wherein the targeting sequence comprises SEQ ID NO: 115.

Embodiment 138. The antisense oligomer, or a pharmaceutically acceptable salt thereof, of any one of embodiments 1-2 or 107-108, wherein the target region is H8D(+12-13).

Embodiment 139. The antisense oligomer, or a pharmaceutically acceptable salt thereof, of any one of embodiments 1-2, 107-108, or 138, wherein the targeting sequence comprises SEQ ID NO: 120.

Embodiment 140. The antisense oligomer, or a pharmaceutically acceptable salt thereof, of embodiment 1 or 2, wherein the target region is an intron/exon junction or exon internal region of exon 9 (SEQ ID NO: 6).

Embodiment 141. The antisense oligomer, or a pharmaceutically acceptable salt thereof, of any one of embodiments 1-2 or 140, wherein the target region is selected from H9A(−5+20), H9A(+1+25), H9A(+51+75), and H9D(+7-18).

Embodiment 142. The antisense oligomer, or a pharmaceutically acceptable salt thereof, of any one of embodiments 1-2 or 140-141, wherein the targeting sequence comprises a sequence selected from SEQ ID NOs: 121-124.

Embodiment 143. The antisense oligomer, or a pharmaceutically acceptable salt thereof, of any one of embodiments 1-2 or 142, wherein the target region is within the 5^{th} nucleotide of intron 8 measured from the 5′ end of exon 9 (SEQ ID NO: 6) and the 25^{th} nucleotide of exon 9 measured from 5′ end of exon 9 (SEQ ID NO: 6) of the human UMOD gene pre-mRNA.

Embodiment 144. The antisense oligomer, or a pharmaceutically acceptable salt thereof, of any one of embodiments 1-2, 140-141, or 143, wherein the target region is selected from H9A(−5+20) and H9A(+1+25).

Embodiment 145. The antisense oligomer, or a pharmaceutically acceptable salt thereof, of any one of embodiments 1-2, 140-141, or 143-144, wherein the targeting sequence comprises a sequence selected from SEQ ID NOs: 121 and 122.

Embodiment 146. The antisense oligomer, or a pharmaceutically acceptable salt thereof, of any one of embodiments 1-2 or 140-141, wherein the target region is H9A(+51+75).

Embodiment 147. The antisense oligomer, or a pharmaceutically acceptable salt thereof, of any one of embodiments 1-2, 140-141, or 146, wherein the targeting sequence comprises SEQ ID NO: 123.

Embodiment 148. The antisense oligomer, or a pharmaceutically acceptable salt thereof, of any one of embodiments 1-2 or 140-141, wherein the target region is H9D(+7-18).

Embodiment 149. The antisense oligomer, or a pharmaceutically acceptable salt thereof, of any one of embodiments 1-2, 140-141, or 148, wherein the targeting sequence comprises SEQ ID NO: 124.

Embodiment 150. The antisense oligomer, or a pharmaceutically acceptable salt thereof, of any one of embodiments 1-149, wherein the antisense oligomer, or the pharmaceutically acceptable salt thereof, is selected from a peptide nucleic acid, a locked nucleic acid, a phosphorodiamidate morpholino oligomer, a 2′—OMe phosphorothioate oligomer, or a combination thereof.

Embodiment 151. The antisense oligomer, or a pharmaceutically acceptable salt thereof, of any one of embodiments 1-150, wherein the antisense oligomer, or the pharmaceutically acceptable salt thereof, is a phosphorodiamidate morpholino oligomer (PMO).

Embodiment 152. The antisense oligomer, or a pharmaceutically acceptable salt thereof, of any one of embodiments 1-151, wherein the antisense oligomer, or the pharmaceutically acceptable salt thereof, further comprises a delivery agent selected from a cell-penetrating peptide, an antibody, a fragment of an antibody, an antigen binding agent, at least one ligand, and a combination thereof.

Embodiment 153. The antisense oligomer, or a pharmaceutically acceptable salt thereof, of any one of embodiments 1-152, wherein the antisense oligomer, or the pharmaceutically acceptable salt thereof, is covalently linked to a cell-penetrating peptide.

Embodiment 154. The antisense oligomer, or a pharmaceutically acceptable salt thereof, of embodiment 153, wherein the antisense oligomer, or the pharmaceutically acceptable salt thereof, is covalently linked to the cell-penetrating peptide via a linker selected from a direct bond, a glycine amino acid, a proline amino acid, glutamic acid amino acid, or an isoglutamine amino acid.

Embodiment 155. The antisense oligomer, or a pharmaceutically acceptable salt thereof, of embodiment 153 or 154, wherein the cell-penetrating peptide is selected from rTAT, TAT, R₉F₂, R₅F₂R₄, R₄, R₅, R₆, R₇, R₈, R₉, (RXR)₄, (RXR)₅, (RXRRBR)₂, (RAR)₄F₂, (RGR)₄F₂, and RBRBYLIQFRBRRBR, wherein A represents alanine, B represents beta-alanine (also represented as β-Ala or β), F represents phenylalanine, G represents glycine, I represents isoleucine, L represents leucine, Q represents glutamine, R represents arginine, X represent 6-aminohexanoic acid (also represented as Ahx or α), and Y represents tyrosine.

Embodiment 156. The antisense oligomer of any one of embodiments 1-155 having a structure of Formula (I):

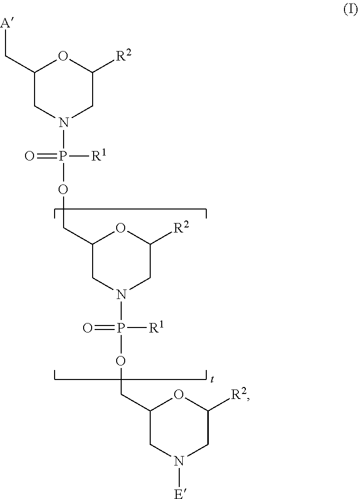

or a pharmaceutically acceptable salt thereof,
wherein:
A′ is selected from —OH,

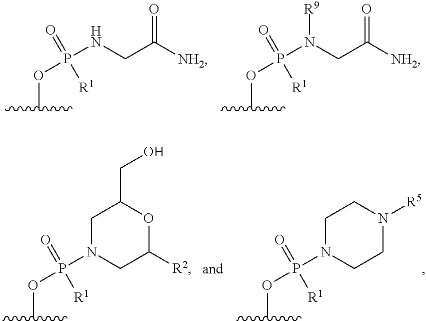

wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, —C₁₋₆-aryl-R⁶, 5-10 membered heteroaryl-R⁶, —C(O)O—(C₁₋₆-alkyl)-R⁶, —C(O)O-aryl-R⁶, —C(O)O-(5-10 membered heteroaryl)-R⁶, and

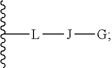

R⁶ is selected from —OH, —SH, and —NH₂, or R⁶ is O, S, or NH, each of which is covalently linked to a solid support;
R⁹ is C₁₋₆ alkyl;
each R¹ is independently selected from —OH and —N(R³)(R⁴), wherein each R³ and R⁴ is, independently at each occurrence, —H or —C₁₋₆-alkyl;
each R² is independently selected from a naturally or non-naturally occurring nucleobase, which, when taken together, forms the targeting sequence;
t is 11-28;
E′ is selected from —H, —C₁₋₆-alkyl, —C(O)C₁₋₆-alkyl, benzoyl, stearoyl, trityl, monomethoxytrityl, dimethoxytrityl, trimethoxytrityl,

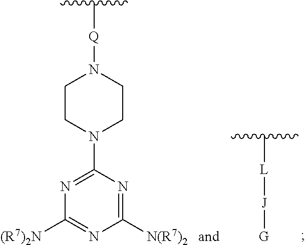

wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH) NH₂;
L is a linking amino acid, wherein L is covalently-linked by an amide bond to the C-terminus of J;
J is a cell-penetrating peptide; and
G is selected from —H, —C(O)C₁₋₆-alkyl, benzoyl, and stearoyl, wherein G is covalently-linked to J.

Embodiment 157. The antisense oligomer, or a pharmaceutically acceptable salt thereof, of embodiment 156, wherein E′ is selected from —H, —C₁₋₆-alkyl, —C(O)C₁₋₆-alkyl, benzoyl, stearoyl, trityl, monomethoxytrityl, dimethoxytrityl, trimethoxytrityl, and

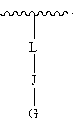

Embodiment 158. The antisense oligomer, or a pharmaceutically acceptable salt thereof, of embodiment 156 or 157, wherein E′ is selected from —H, —C(O) CH₃, benzoyl, stearoyl, trityl, 4-methoxytrityl, and

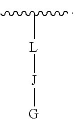

Embodiment 159. The antisense oligomer, or a pharmaceutically acceptable salt thereof, of any one of embodiments 156-158, wherein A′ is selected from:

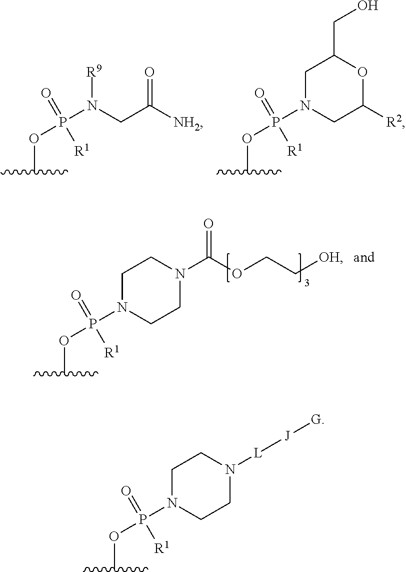

Embodiment 160. The antisense oligomer, or a pharmaceutically acceptable salt thereof, of any one of embodiments 156-159, wherein at least one of the following is true:
(1) A′ is

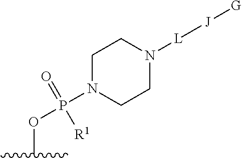

or (2) E′ is

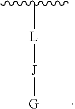

Embodiment 161. The antisense oligomer, or a pharmaceutically acceptable salt thereof, of any one of embodiments 156-160, wherein A′ is selected from

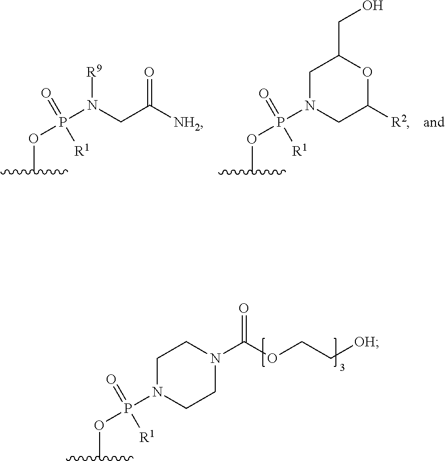

and E′ is

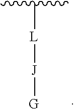

Embodiment 162. The antisense oligomer, or a pharmaceutically acceptable salt thereof, of any one of embodiments 156-160, wherein A′ is

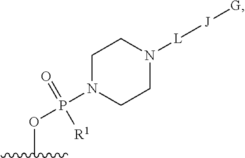

and
E′ is selected from H, —C(O) CH₃, trityl, 4-methoxytrityl, benzoyl, and stearoyl.

Embodiment 163. The antisense oligomer, or a pharmaceutically acceptable salt thereof, of any one of embodiments 156-162, wherein each R¹ is —N(CH₃)₂.

Embodiment 164. The antisense oligomer, or a pharmaceutically acceptable salt thereof, of any one of embodiments 156-163, wherein L is glycine, proline, or β-alanine.

Embodiment 165. The antisense oligomer, or a pharmaceutically acceptable salt thereof, of any one of embodiments 156-164, wherein L is glycine.

Embodiment 166. The antisense oligomer, or a pharmaceutically acceptable salt thereof, of any one of embodiments 156-164, wherein L is proline.

Embodiment 167. The antisense oligomer, or a pharmaceutically acceptable salt thereof, of any one of embodiments 156-164, wherein L is β-alanine.

Embodiment 168. The antisense oligomer, or a pharmaceutically acceptable salt thereof, of any one of embodiments 156-167, wherein J is selected from rTAT, TAT, R₉F₂, R₅F₂R₄, R₄, R₅, R₆, R₇, R₈, R₉, (RXR)₄, (RXR)₅, (RXRRBR)₂, (RAR)₄F₂, and (RGR)₄F₂, wherein A represents alanine, B represents β-alanine (also represented as β-Ala or β), F represents phenylalanine, G represented glycine, R represents arginine, and X represent 6-aminohexanoic acid (also represented as Ahx or (x).

Embodiment 169. The antisense oligomer, or a pharmaceutically acceptable salt thereof, of any one of embodiments 156-168, wherein G is selected from —H, —C(O) CH₃, benzoyl, and stearoyl.

Embodiment 170. The antisense oligomer, or a pharmaceutically acceptable salt thereof, of any one of embodiments 156-169, wherein G is-H or —C(O) CH₃.

Embodiment 171. The antisense oligomer, or a pharmaceutically acceptable salt thereof, of any one of embodiments 156-170, wherein G is-H.

Embodiment 172. The antisense oligomer, or a pharmaceutically acceptable salt thereof, of any one of embodiments 156-170, wherein G is —C(O)CH₃.

Embodiment 173. An antisense oligomer of any one of embodiments 156-172, of structural Formula (IA):

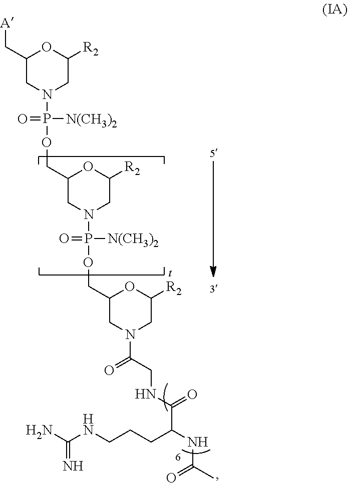

or a pharmaceutically acceptable salt thereof, wherein:
A′ is a moiety selected from:

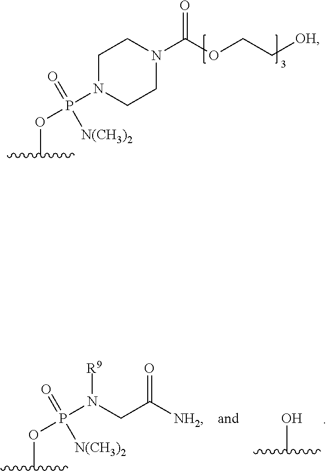

Embodiment 174. The antisense oligomer of any one of embodiments 156-172, of structural Formula (II):

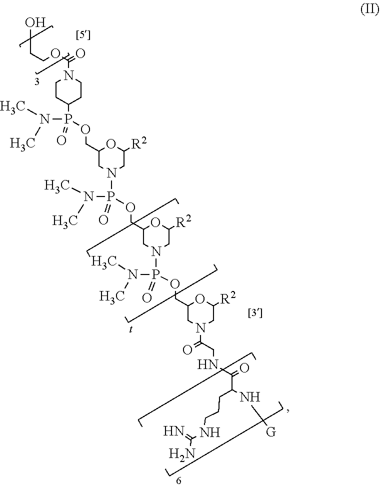

or a pharmaceutically acceptable salt thereof.

Embodiment 175. The antisense oligomer of any one of embodiments 156-172, of structural Formula (III):

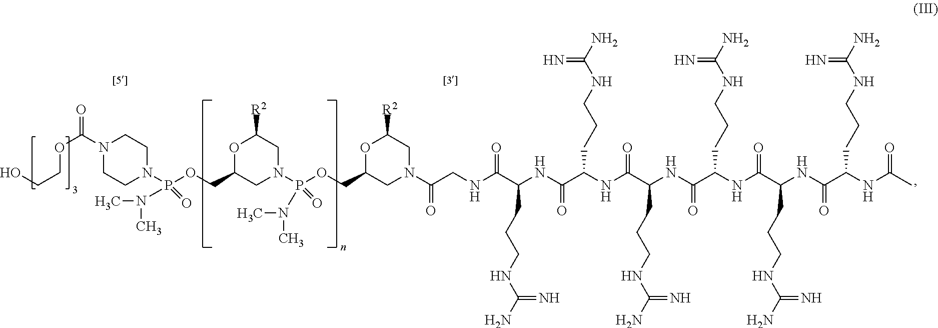

or a pharmaceutically acceptable salt thereof, wherein n is 11-28.

Embodiment 176. The antisense oligomer of any one of embodiments 156-172 of structural Formula (IV):

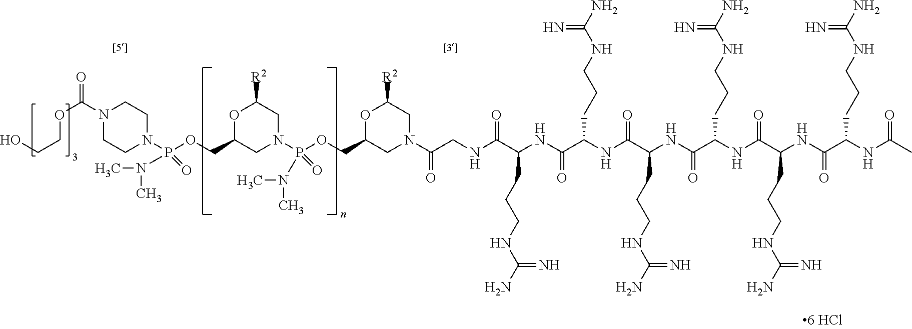

(IV), wherein n is 11-28.

Embodiment 177. The antisense oligomer, or the pharmaceutically acceptable salt thereof, of embodiment 175 or 176, or a pharmaceutically acceptable salt thereof, wherein each R₂, taken together, forms a targeting sequence having SEQ ID NO: 37.

Embodiment 178. The antisense oligomer, or the pharmaceutically acceptable salt thereof, of embodiment 175 or 176, or a pharmaceutically acceptable salt thereof, wherein each R², taken together, forms a targeting sequence having SEQ ID NO: 32.

Embodiment 179. The antisense oligomer, or the pharmaceutically acceptable salt thereof, of embodiment 175 or 176, or a pharmaceutically acceptable salt thereof, wherein each R², taken together, forms a targeting sequence having SEQ ID NO: 70.

Embodiment 180. The antisense oligomer, or the pharmaceutically acceptable salt thereof, of embodiment 175 or 176, or a pharmaceutically acceptable salt thereof, wherein each R², taken together, forms a targeting sequence having SEQ ID NO: 74.

Embodiment 181. The antisense oligomer, or the pharmaceutically acceptable salt thereof, of embodiment 175 or 176, or a pharmaceutically acceptable salt thereof, wherein each R², taken together, forms a targeting sequence having SEQ ID NO: 62.

Embodiment 182. The antisense oligomer, or the pharmaceutically acceptable salt thereof, of embodiment 175 or 176, or a pharmaceutically acceptable salt thereof, wherein each R², taken together, forms a targeting sequence having SEQ ID NO: 81.

Embodiment 183. The antisense oligomer, or the pharmaceutically acceptable salt thereof, of embodiment 175 or 176, or a pharmaceutically acceptable salt thereof, wherein each R², taken together, forms a targeting sequence having SEQ ID NO: 114.

Embodiment 184. The antisense oligomer, or the pharmaceutically acceptable salt thereof, of embodiment 175 or 176, or a pharmaceutically acceptable salt thereof, wherein each R², taken together, forms a targeting sequence having SEQ ID NO: 120.

Embodiment 185. An antisense oligomer selected from:

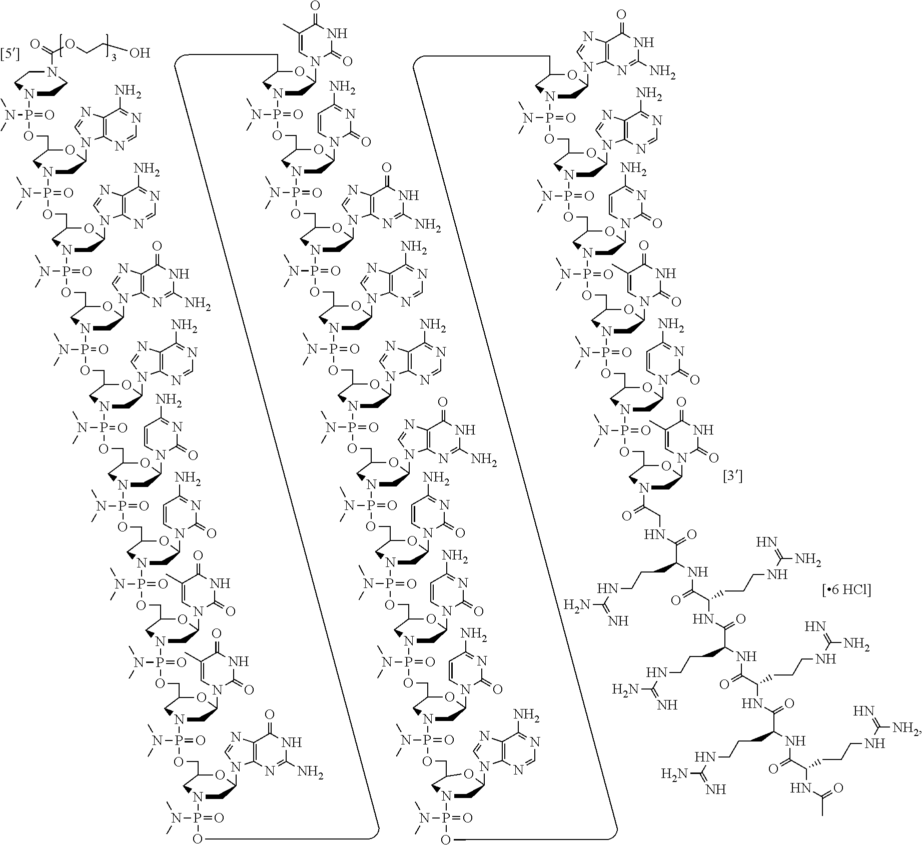

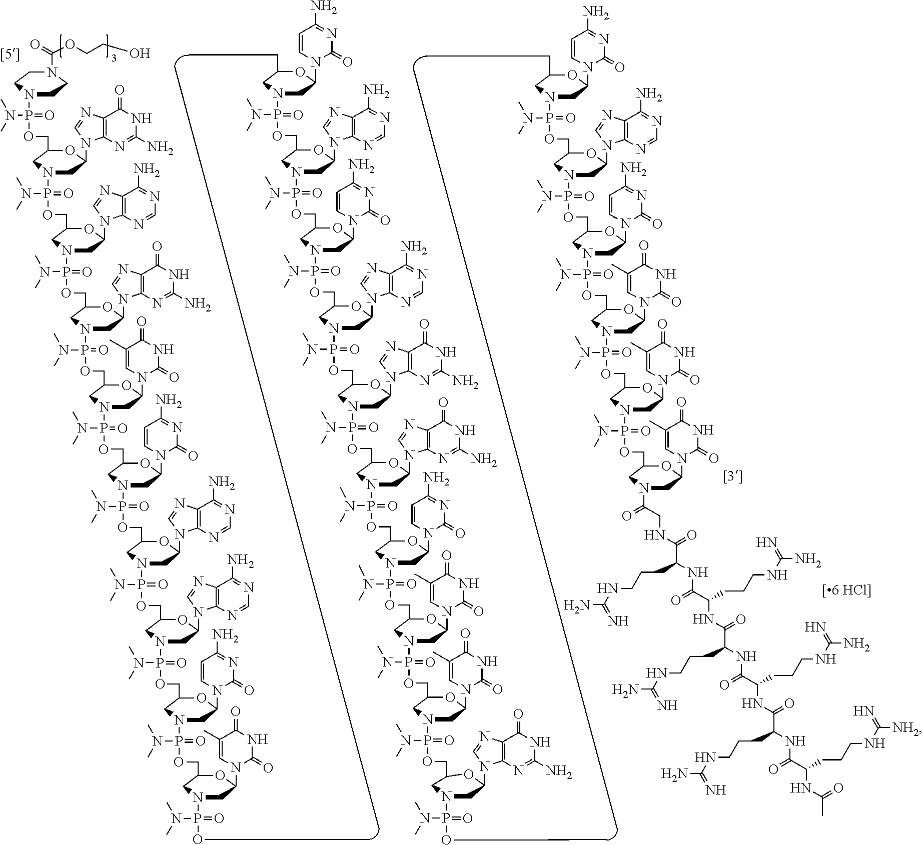

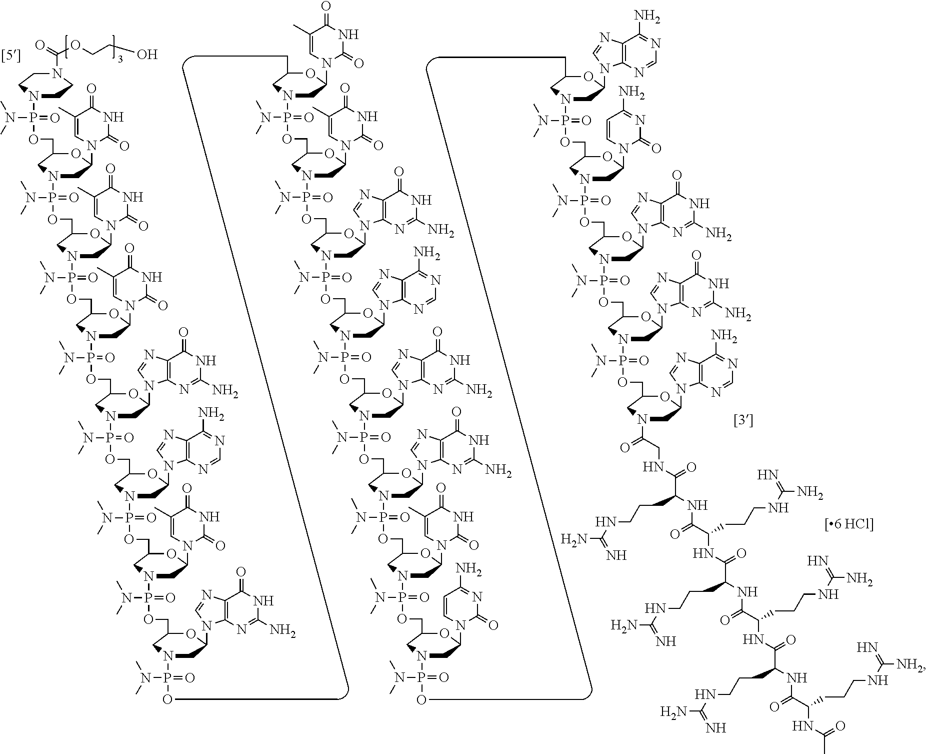

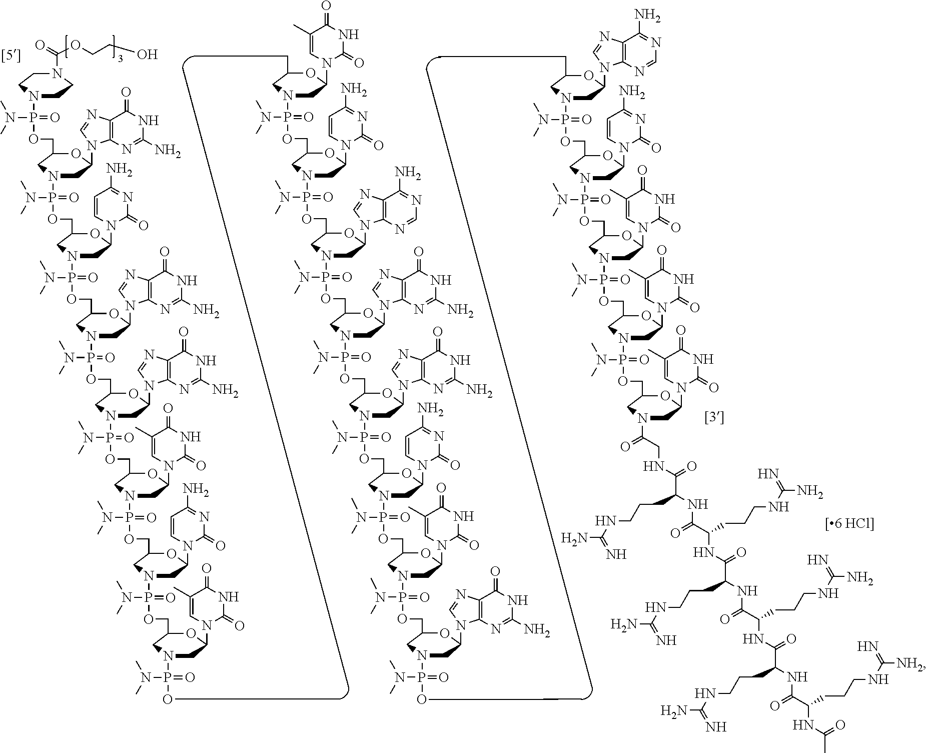

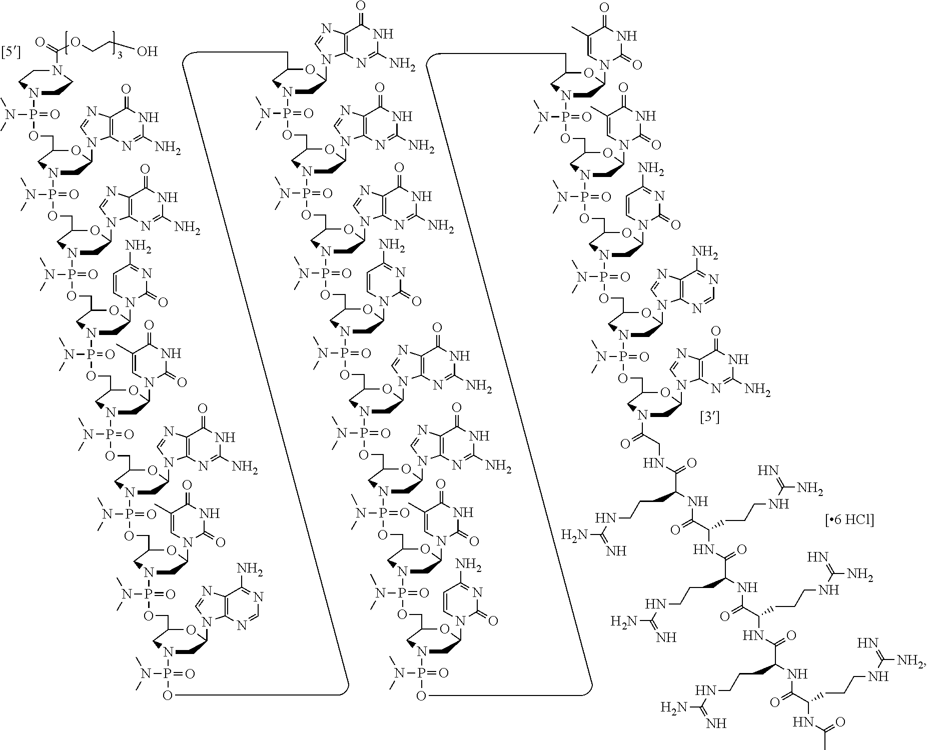

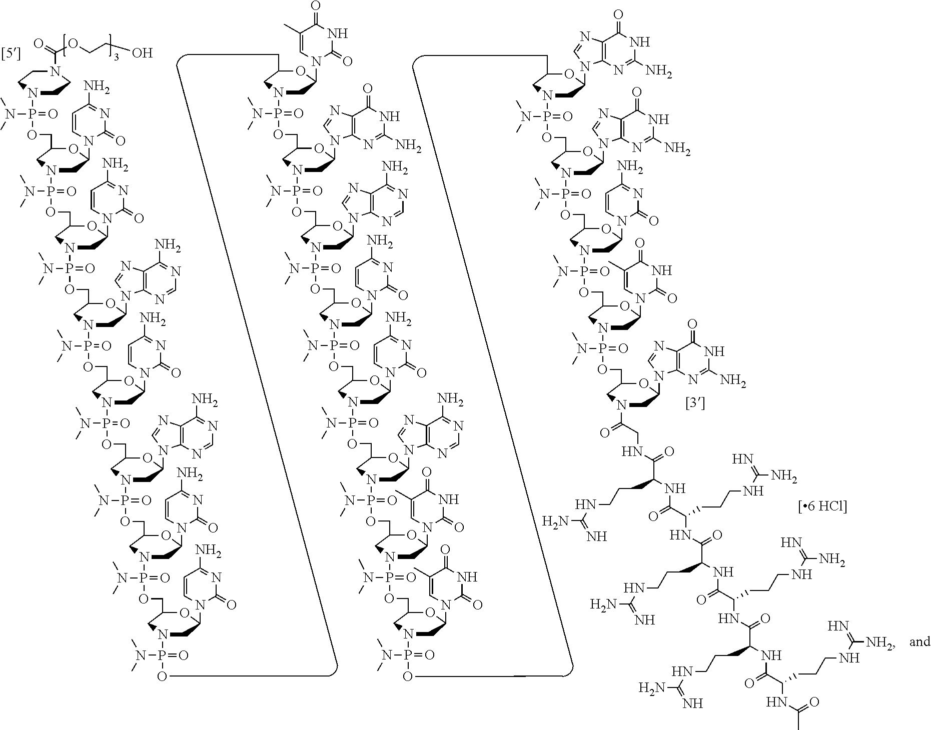

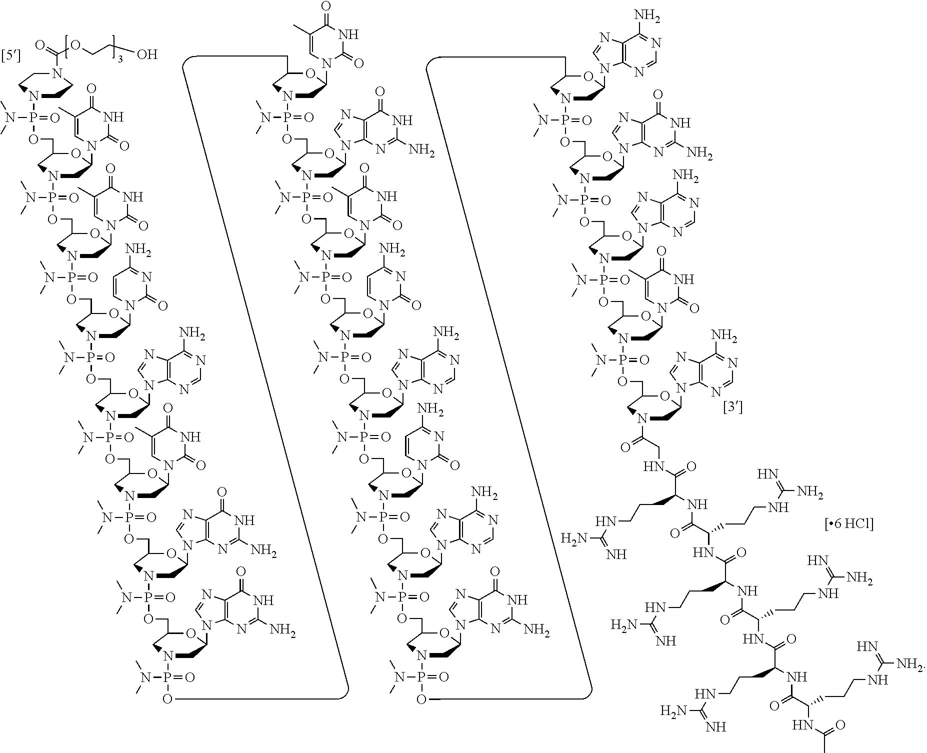

Embodiment 186. The antisense oligomer of embodiment 185 that is:

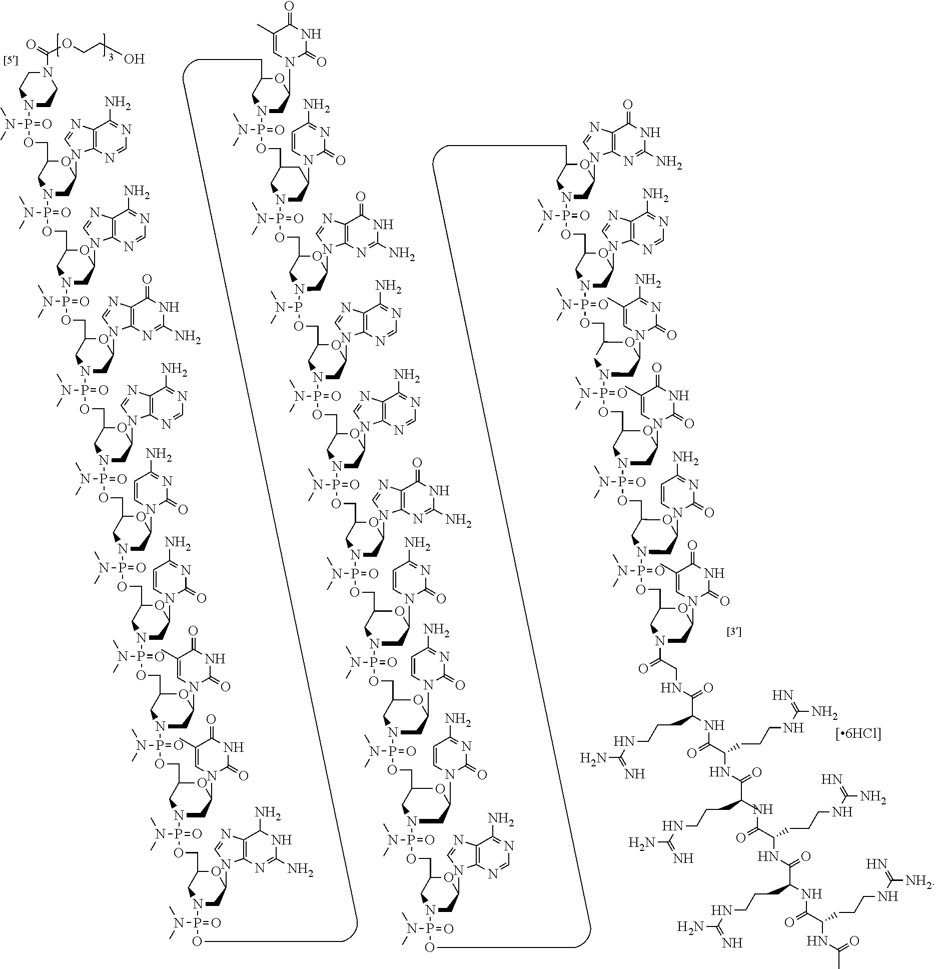

Embodiment 187. The antisense oligomer of embodiment 185 that is:

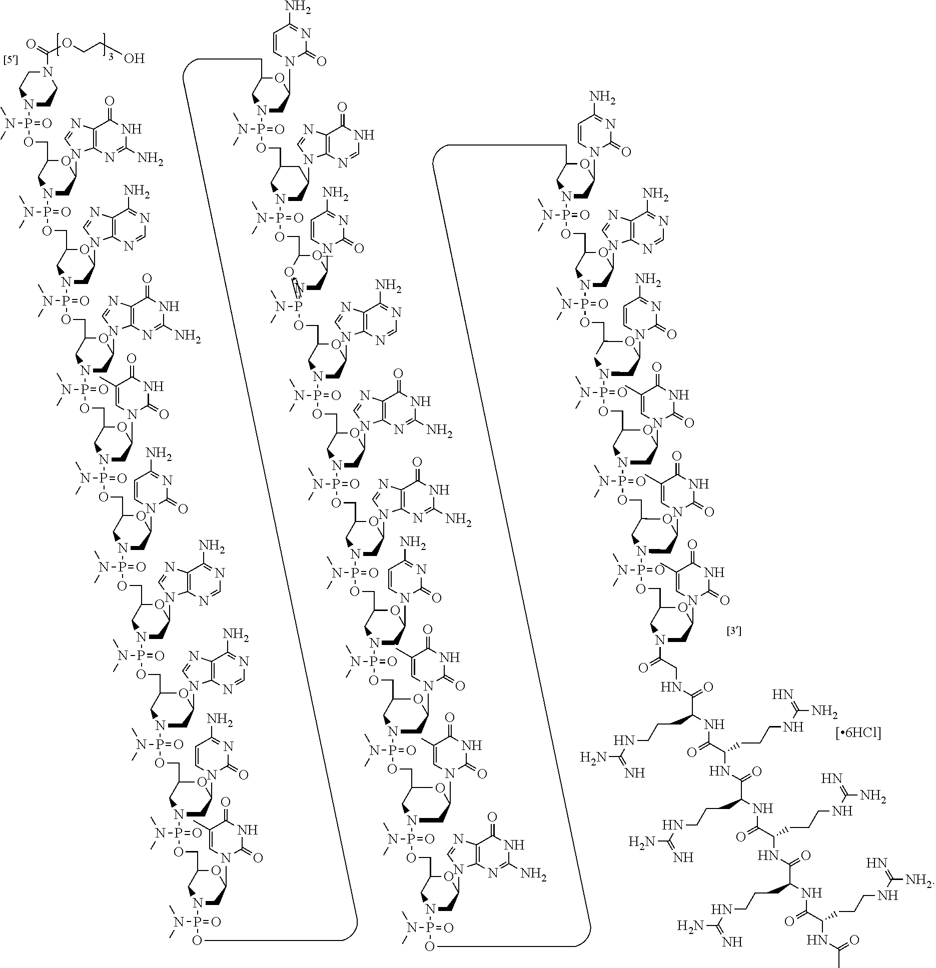

Embodiment 188. The antisense oligomer of embodiment 185 that is:

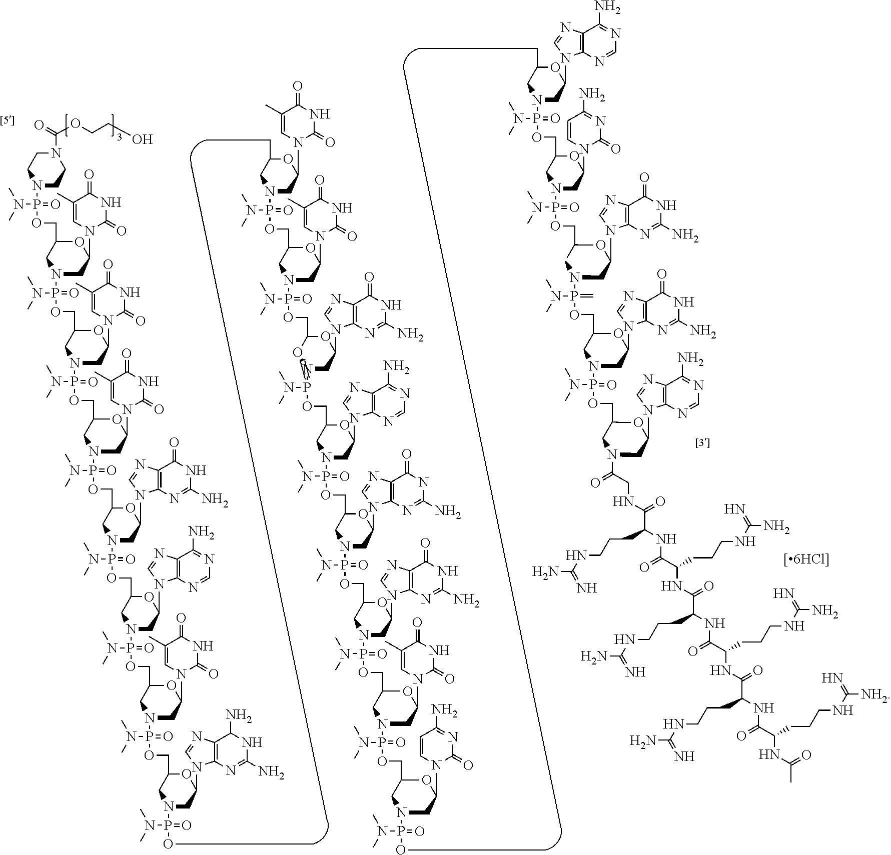

Embodiment 189. The antisense oligomer of embodiment 185 that is:

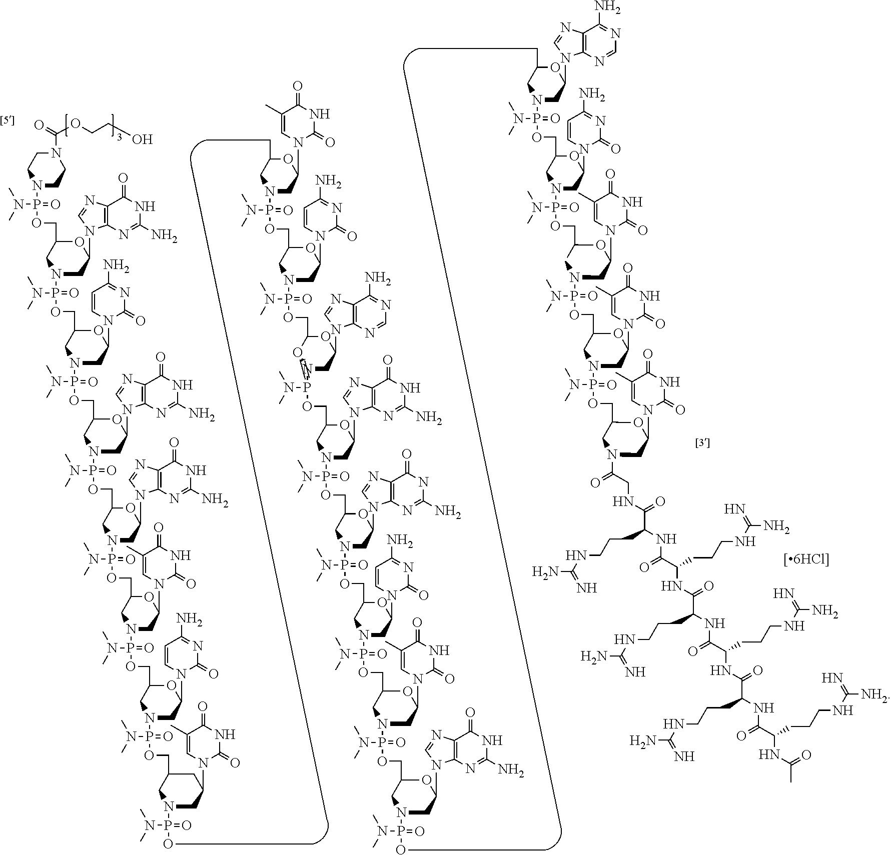

Embodiment 190. The antisense oligomer of embodiment 185 that is:

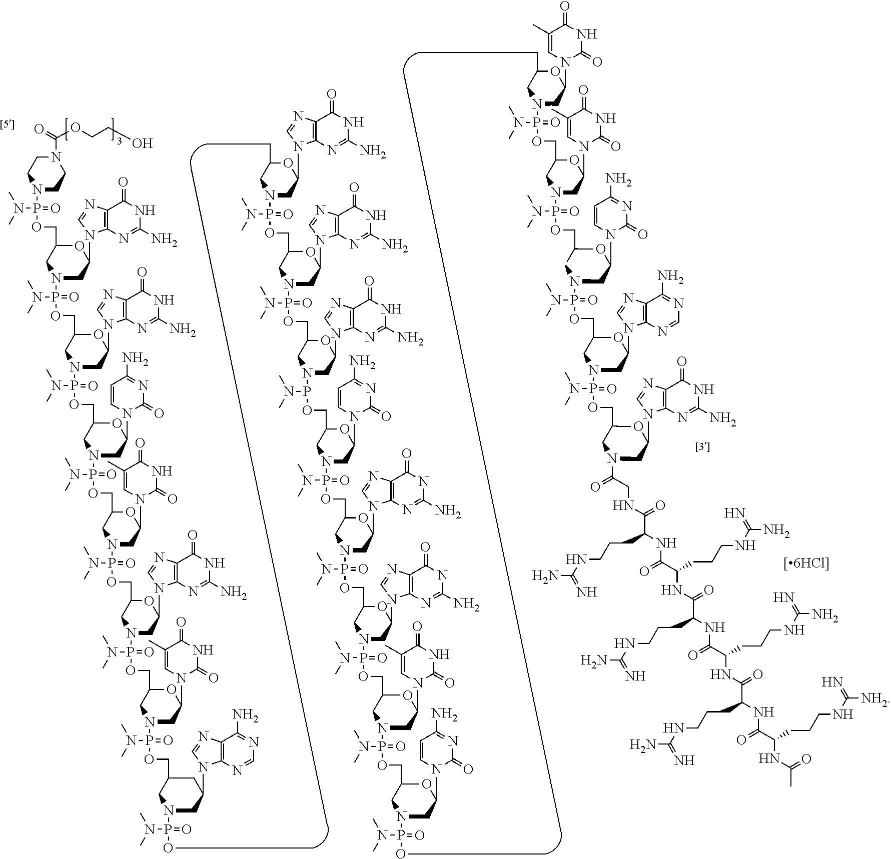

Embodiment 191. The antisense oligomer of embodiment 185 that is:

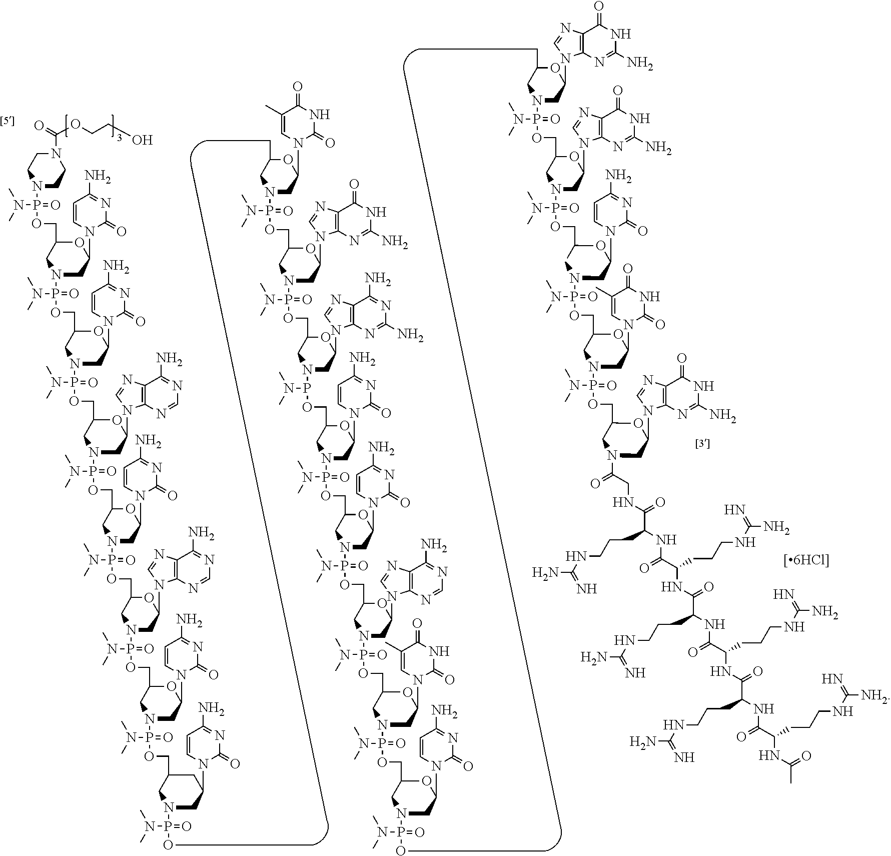

Embodiment 192. The antisense oligomer of embodiment 185 that is:

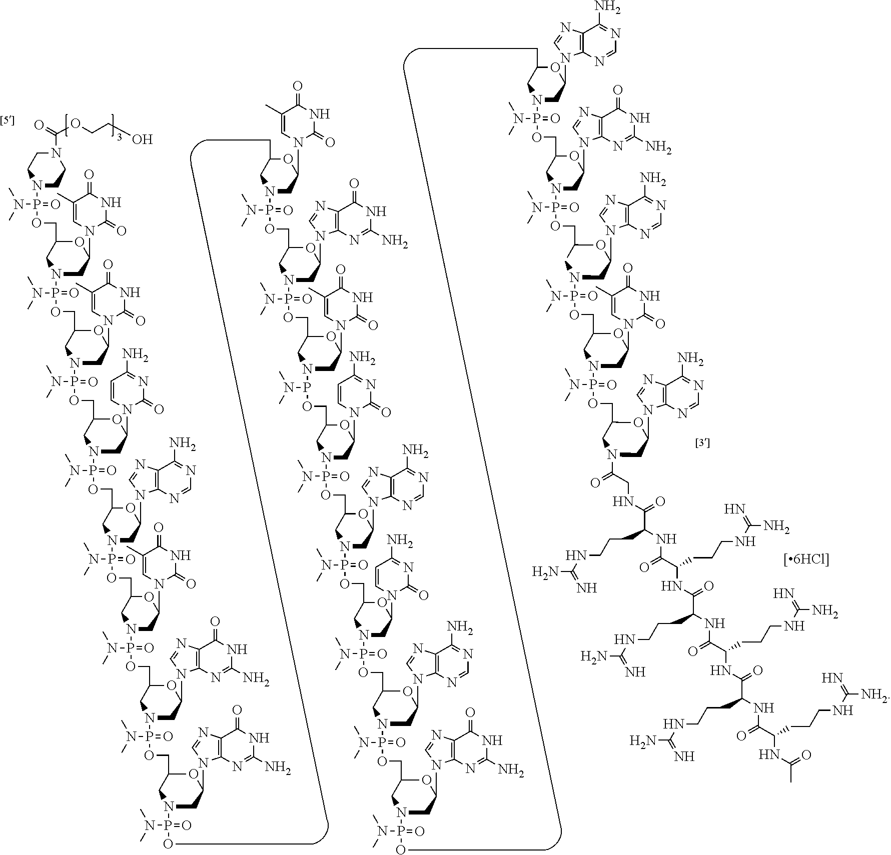

Embodiment 193. A pharmaceutical composition comprising the antisense oligomer, or a pharmaceutically acceptable salt thereof, of any one of embodiments 1-192 and a pharmaceutically acceptable carrier.

Embodiment 194. A method of treating a chronic kidney disease (CKD), comprising administering to a subject in need thereof a therapeutically effective amount of the antisense oligomer, or a pharmaceutically acceptable salt thereof, of any one of embodiments 1-192 or the pharmaceutical composition of embodiment 193.

Embodiment 195. A method of treating a disease associated with aberrant expression of uromodulin protein, comprising administering to a subject in need thereof a therapeutically effective amount of the antisense oligomer, or a pharmaceutically acceptable salt thereof, of any one of embodiments 1-192 or the pharmaceutical composition of embodiment 193.

Embodiment 196. A method of treating a uromodulin-associated renal disease, comprising administering to a subject in need thereof a therapeutically effective amount of the antisense oligomer, or a pharmaceutically acceptable salt thereof, of any one of embodiments 1-192 or the pharmaceutical composition of embodiment 193.

Embodiment 197. The method of any one of embodiments 194-196, wherein the disease is an autosomal dominant renal disorder.

Embodiment 198. The method of embodiment 197, wherein the autosomal dominant renal disorder is an autosomal dominant tubulointerstitial kidney disease (ADTKD).

Embodiment 199. The method of any one of embodiments 194-196, wherein the disease is autosomal dominant tubulointerstitial kidney disease-uromodulin (ADTKD-UMOD).

### EXAMPLES

Examples have been set forth below for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations, or methods of the disclosure may be made without departing from the spirit of the disclosure and the scope of the appended claims. Definitions of the variables in the structures in the schemes herein are commensurate with those of corresponding positions in the formulae presented herein.

### Example 1—Design of Antisense Targeting Sequences

Antisense oligomer targeting sequences were designed for therapeutic applications related to the pre-mRNA of the human UMOD gene. Here, antisense oligomers will induce exon skipping to create a premature stop codon (PTC) and thereby trigger nonsense-mediated decay of UMOD in a diseased cell. Restoration of normal or near-normal UMOD expression would then reduce disease causing aggregation in the cell, thereby providing a clinical benefit to ADTKD-UMOD patients.

Exemplary oligomers comprising a targeting sequence as set forth in Table 5 were prepared as PPMOs (e.g., an antisense oligomer of Formula (IV)). As described below, these antisense oligomers were introduced into human DMS454 cells and mouse mIMCD-3 cells and analyzed for UMOD expression.

### Example 2—PPMO Screen in Human DMS454 Cells

Human DMS545 cells were seeded in 96 well plates for four days before transfection. Cells were transfected with 20 mM of PPMO combined with 1 mM endoporter. Each PPMO comprises a phosphorodiamidate backbone and a targeting sequens of nucleobases as shown in Table 5. The antisense PPMO oligomers were prepared with a 5′ terminus capping group of TEG and a 3′ terminus capping group of -G-R₆, where G is glycine and R is arginine. After a 72-hour incubation period, cells were collected and analyzed. Real time PCR (RT-PCR) was performed on the transfected DMS454 cells, and the % knockdown was calculated to be percentage of total UMOD transcript less than the vehicle control. The knockdown data is shown in Table 5.

TABLE 5
DMS454 Microwalk Data.

| | | Targeting | | |
|---|---|---|---|---|
| | Targeting Sequence of | Sequence of | | |
| | nucleobases | nucleobases | % | PPMO |
| Target Region | (5′-3′) | SEQ ID NO | Knockdown | Name |
| UMOD | CTGTGAACAGAGATGGATGGGACAA | 7 | 0.00% | PPMO2.4 |
| H2A(−25−1) | | | | |
| UMOD | TCCTGTGAACAGAGATGGAT | 8 | 0.00% | PPMO2.1 |
| H2A(−18+2) | | | | |
| UMOD | GTCCTGTGAACAGAGATGGA | 9 | 0.00% | PPMO2.2 |
| H2A(−17+3) | | | | |
| UMOD | TGTCCTGTGAACAGAGATGG | 10 | 0.00% | PPMO2.3 |
| H2A(−16+4) | | | | |
| UMOD | GTCTGGTGTCCTGTGAACAGAGATG | 11 | 17.20% | PPMO2.23 |
| H2A(−15+10) | | | | |
| UMOD | CTCTCTGTCTCTGATGTCTGGTGTC | 12 | 29.50% | PPMO2.5 |
| H2A(+1+25) | | | | |
| UMOD | AGACGGGTTGGCCCTTTGAATTTTT | 13 | 0.80% | PPMO2.6 |
| H2A(+26+50) | | | | |
| UMOD | TCTAGATAGCACCTGCCCAAAGGAA | 14 | 15.10% | PPMO2.7 |
| H2A(+51+75) | | | | |
| UMOD | ATCCTTTCTGCTCTTCCCGC | 15 | 12.30% | PPMO2.8 |
| H2A(+85+104) | | | | |
| UMOD | CATCCTTTCTGCTCTTCCCG | 16 | 13.10% | PPMO2.9 |
| H2A(+86+105) | | | | |
| UMOD | AGAGATGGCTGCCCCATCCTTTCTG | 17 | 1.40% | PPMO2.10 |
| H2A(+95+119) | | | | |
| UMOD | ATGGCTGCCCCATCCTTTCT | 18 | 0.00% | PPMO2.11 |
| H2A(+96+115) | | | | |
| UMOD | AGATGGCTGCCCCATCCTTT | 19 | 0.00% | PPMO2.12 |
| H2A(+98+117) | | | | |
| UMOD | CAAGTCAGAGATGGCTGCCCCATCC | 20 | 12.90% | PPMO2.13 |
| H2A(+101+125) | | | | |
| UMOD | TCCAAGTCAGAGATGGCTGC | 21 | 0.00% | PPMO2.14 |
| H2A(+108+127) | | | | |
| UMOD | ATCCAAGTCAGAGATGGCTG | 22 | 0.00% | PPMO2.15 |
| H2A(+109+128) | | | | |
| UMOD | CATCCAAGTCAGAGATGGCT | 23 | 28.20% | PPMO2.16 |
| H2A(+110+129) | | | | |
| UMOD | CAGCATCCAAGTCAGAGATG | 24 | 0.00% | PPMO2.17 |
| H2A(+113+132) | | | | |
| UMOD | TCAGCATCCAAGTCAGAGAT | 25 | 0.00% | PPMO2.18 |
| H2A(+114+133) | | | | |
| UMOD | ACCATCAGCATCCAAGTCAG | 26 | 1.30% | PPMO2.19 |
| H2A(+118+137) | | | | |
| UMOD | AGAGGCCACCACCACCATCAGCATC | 27 | 5.60% | PPMO2.20 |
| H2A(+126+150) | | | | |
| UMOD | CAGTGGCTGCAGTTGTGATGAACCA | 28 | 7.60% | PPMO2.21 |
| H2A(+151+175) | | | | |
| UMOD | TTTCACTTACTTGCTTCTGAGGTGT | 29 | 0.00% | PPMO2.22 |
| H2D(+15−10) | | | | |
| UMOD | GATATCTGAAACAGGTTAGG | 30 | 31.00% | PPMO5.1 |
| H5A(−15+5) | | | | |
| UMOD | AGATATCTGAAACAGGTTAG | 31 | 37.80% | PPMO5.2 |
| H5A(−14+6) | | | | |
| UMOD | GGGAGATATCTGAAACAGGT | 32 | 39.80% | PPMO5.3 |
| H5A(−11+9) | | | | |
| UMOD | AGGGAGATATCTGAAACAGG | 33 | 53.90% | PPMO5.4 |
| H5A(−10+10) | | | | |
| UMOD | GAGGGAGATATCTGAAACAG | 125 | 0.00% | PPMO5.18 |
| H5A(−9+11) | | | | |
| UMOD | CACTTGCCCAGCGACACC | 34 | 0.00% | PPMO5.6 |
| H5A(+50+67) | | | | |
| UMOD | TCAGCTGGCACTTGCCCAGCGACAC | 35 | 31.90% | PPMO5.19 |
| H5A(+51+75) | | | | |
| UMOD | GGCACTTGCCCAGCGACA | 36 | 0.00% | PPMO5.7 |
| H5A(+52+69) | | | | |
| UMOD | AAGACCTTGTCGAAGCCCAGACTCT | 37 | 47.50% | PPMO5.20 |
| H5A(+76+100) | | | | |
| UMOD | CTTGTCGAAGCCCAGACT | 38 | 27.00% | PPMO5.8 |
| H5A(+78+95) | | | | |
| UMOD | ACCTTGTCGAAGCCCAGA | 39 | 29.20% | PPMO5.9 |
| H5A(+80+97) | | | | |
| UMOD | AGACCTTGTCGAAGCCCA | 40 | 37.20% | PPMO5.10 |
| H5A(+82+99) | | | | |
| UMOD | TGAAGACCTTGTCGAAGC | 41 | 0.00% | PPMO5.11 |
| H5A(+85+102) | | | | |
| UMOD | ATGAAGACCTTGTCGAAG | 42 | 0.60% | PPMO5.12 |
| H5A(+86+103) | | | | |
| UMOD | GGTACATGAAGACCTTGT | 43 | 0.00% | PPMO5.13 |
| H5A(+91+108) | | | | |
| UMOD | GGTTGTCTCTGTCATTGAAGCCCGA | 44 | 5.00% | PPMO5.21 |
| H5A(+126+150) | | | | |
| UMOD | TCACTACAGACACCCAGTCC | 45 | 0.00% | PPMO5.14 |
| H5A(+152+171) | | | | |
| UMOD | GTCACTACAGACACCCAGTC | 46 | 12.90% | PPMO5.15 |
| H5A(+153+172) | | | | |
| UMOD | GGTCACTACAGACACCCAGT | 47 | 40.40% | PPMO5.16 |
| H5A(+154+173) | | | | |
| UMOD | ACCGTCAACACTGTCCCACA | 48 | 11.70% | PPMO5.24 |
| H5D(+18−2) | | | | |
| UMOD | TACCGTCAACACTGTCCCAC | 49 | 0.00% | PPMO5.23 |
| H5D(+17−3) | | | | |
| UMOD | GTACCGTCAACACTGTCCCA | 50 | 8.90% | PPMO5.22 |
| H5D(+16−4) | | | | |
| UMOD | ACGTACCGTCAACACTGTCC | 51 | 21.80% | PPMO5.21 |
| H5D(+14−6) | | | | |
| UMOD | GACGTACCGTCAACACTGTC | 52 | 23.00% | PPMO5.20 |
| H5D(+13−7) | | | | |
| UMOD | GGACGTACCGTCAACACTGT | 53 | 34.90% | PPMO5.19 |
| H5D(+12−8) | | | | |
| UMOD | AGGACGTACCGTCAACACTG | 54 | 0.00% | PPMO5.18 |
| H5D(+11−9) | | | | |
| UMOD | CAGGACGTACCGTCAACACT | 55 | 0.00% | PPMO5.17 |
| H5D(+10−10) | | | | |
| UMOD | TGTAAGTGGCATGGGTTTCATTCCT | 56 | 0.00% | PPMO6.1 |
| H6A(+1+25) | | | | |
| UMOD | TCATCTGCCAGGTAGAGGGTGTTGC | 57 | 34.80% | PPMO6.24 |
| H6A(+26+50) | | | | |
| UMOD | GGTCACGGATGATGATCTCA | 58 | 31.40% | PPMO6.2 |
| H6A(+48+67) | | | | |
| UMOD | AGGTCACGGATGATGATCTC | 59 | 54.90% | PPMO6.3 |
| H6A(+49+68) | | | | |
| UMOD | GAGGTCACGGATGATGATCT | 60 | 0.00% | PPMO6.4 |
| H6A(+50+69) | | | | |
| UMOD | TTGATGTTGAGGTCACGGAT | 61 | 52.10% | PPMO6.5 |
| H6A(+58+77) | | | | |
| UMOD | TTTGATGTTGAGGTCACGGA | 62 | 70.20% | PPMO6.6 |
| H6A(+59+78) | | | | |
| UMOD | TTTTGATGTTGAGGTCACGG | 63 | 0.00% | PPMO6.7 |
| H6A(+60+79) | | | | |
| UMOD | GGTAGGAGCATGCAAAGTTGATTTT | 64 | 42.00% | PPMO6.25 |
| H6A(+76+100) | | | | |
| UMOD | TAGGAGCATGCAAAGTTGAT | 65 | 0.00% | PPMO6.8 |
| H6A(+79+98) | | | | |
| UMOD | TTCAGGCTGACTTTCATGTCCAGGG | 66 | 50.30% | PPMO6.26 |
| H6A(+101+125) | | | | |
| UMOD | GCTGACTTTCATGTCCAGGG | 67 | 31.20% | PPMO6.9 |
| H6A(+101+120) | | | | |
| UMOD | GGTCTTCAGGCTGACTTTCA | 68 | 36.70% | PPMO6.10 |
| H6A(+110+129) | | | | |
| UMOD | CGGTCTTCAGGCTGACTTTC | 69 | 39.40% | PPMO6.11 |
| H6A(+111+130) | | | | |
| UMOD | GCGGTCTTCAGGCTGACTTT | 70 | 49.90% | PPMO6.12 |
| H6A(+112+131) | | | | |
| UMOD | GGCGGTCTTCAGGCTGACTT | 71 | 34.40% | PPMO6.14 |
| H6A(+113+132) | | | | |
| UMOD | CTGTAGGGCGGTCTTCAGGC | 72 | 45.90% | PPMO6.15 |
| H6A(+119+138) | | | | |
| UMOD | GCTGTAGGGCGGTCTTCAGG | 73 | 57.60% | PPMO6.16 |
| H6A(+120+139) | | | | |
| UMOD | GGCTGTAGGGCGGTCTTCAG | 74 | 61.00% | PPMO6.17 |
| H6A(+121+140) | | | | |
| UMOD | TGGCTGTAGGGCGGTCTTCA | 75 | 26.10% | PPMO6.18 |
| H6A(+122+141) | | | | |
| UMOD | TTGGCTGTAGGGCGGTCTTC | 76 | 41.10% | PPMO6.19 |
| H6A(+123+142) | | | | |
| UMOD | ATTGGCTGTAGGGCGGTCTT | 77 | 22.20% | PPMO6.20 |
| H6A(+124+143) | | | | |
| UMOD | CTGACCATTGGCTGTAGGGC | 78 | 35.80% | PPMO6.21 |
| H6A(+130+149) | | | | |
| UMOD | CCTGACCATTGGCTGTAGGGCGGTC | 79 | 45.30% | PPMO6.27 |
| H6D(+24−1) | | | | |
| UMOD | CACACCTGACCATTGGCTGT | 80 | 51.80% | PPMO6.22 |
| H6D(+15−5) | | | | |
| UMOD | CCACACCTGACCATTGGCTG | 81 | 56.80% | PPMO6.23 |
| H6D(+14−6) | | | | |
| UMOD | TTGAGTCTCTAGTGTGTGGGCATCT | 82 | 19.70% | PPMO8.1 |
| H8A(−2+23) | | | | |
| UMOD | ACTCCCCATTCTCCACCACTTGGAT | 83 | 0.00% | PPMO8.2 |
| H8A(+26+50) | | | | |
| UMOD | TGGACGGAAAATCGGCCCTGGGAGG | 84 | 7.10% | PPMO8.3 |
| H8A(+51+75) | | | | |
| UMOD | CATCTGGACGGAAAATCGGC | 85 | 39.40% | PPMO8.4 |
| H8A(+60+79) | | | | |
| UMOD | ACATCTGGACGGAAAATCGG | 86 | 48.80% | PPMO8.5 |
| H8A(+61+80) | | | | |
| UMOD | AACATCTGGACGGAAAATCG | 87 | 0.00% | PPMO8.6 |
| H8A(+62+81) | | | | |
| UMOD | GAACATCTGGACGGAAAATC | 88 | 0.00% | PPMO8.7 |
| H8A(+63+82) | | | | |
| UMOD | AACCGGAACATCTGGACGGA | 89 | 18.90% | PPMO8.8 |
| H8A(+68+87) | | | | |
| UMOD | AAACCGGAACATCTGGACGG | 90 | 1.90% | PPMO8.9 |
| H8A(+69+88) | | | | |
| UMOD | CAAACCGGAACATCTGGACG | 91 | 20.50% | PPMO8.10 |
| H8A(+70+89) | | | | |
| UMOD | TTCCAGCAAACCGGAACATC | 92 | 17.30% | PPMO8.11 |
| H8A(+76+95) | | | | |
| UMOD | ATAGTTTCCAGCAAACCGGAACATC | 93 | 28.70% | PMO8.38 |
| H8A(+76+100) | | | | |
| UMOD | TTTCCAGCAAACCGGAACAT | 94 | 7.60% | PPMO8.12 |
| H8A(+77+96) | | | | |
| UMOD | GTTTCCAGCAAACCGGAACA | 95 | 11.30% | PPMO8.13 |
| H8A(+78+97) | | | | |
| UMOD | AGTTTCCAGCAAACCGGAAC | 96 | 14.70% | PPMO8.14 |
| H8A(+79+98) | | | | |
| UMOD | TAGTTTCCAGCAAACCGGAA | 97 | 0.00% | PPMO8.15 |
| H8A(+80+99) | | | | |
| UMOD | ATAGTTTCCAGCAAACCGGA | 98 | 3.00% | PPMO8.16 |
| H8A(+81+100) | | | | |
| UMOD | CATAGTTTCCAGCAAACCGG | 99 | 20.90% | PPMO8.17 |
| H8A(+82+101) | | | | |
| UMOD | TCATAGTTTCCAGCAAACCG | 100 | 42.20% | PPMO8.18 |
| H8A(+83+102) | | | | |
| UMOD | AGGTCATAGTTTCCAGCAAA | 101 | 2.00% | PPMO8.19 |
| H8A(+86+105) | | | | |
| UMOD | GGTAGACTAGGTCATAGTTT | 102 | 40.90% | PPMO8.20 |
| H8A(+94+113) | | | | |
| UMOD | AGGTAGACTAGGTCATAGTT | 103 | 33.60% | PPMO8.21 |
| H8A(+95+114) | | | | |
| UMOD | CAGGTAGACTAGGTCATAGT | 104 | 31.50% | PPMO8.22 |
| H8A(+96+115) | | | | |
| UMOD | CTTCACAGTGCAGGTAGACTAGGTC | 105 | 24.00% | PMO8.39 |
| H8A(+101+125) | | | | |
| UMOD | ACAGTGCAGGTAGACTAGGT | 106 | 35.40% | PPMO8.23 |
| H8A(+102+121) | | | | |
| UMOD | CACAGTGCAGGTAGACTAGG | 107 | 35.90% | PPMO8.24 |
| H8A(+103+122) | | | | |
| UMOD | TCACAGTGCAGGTAGACTAG | 108 | 15.70% | PPMO8.25 |
| H8A(+104+123) | | | | |
| UMOD | TTCACAGTGCAGGTAGACTA | 109 | 25.70% | PPMO8.26 |
| H8A(+105+124) | | | | |
| UMOD | GTCACAGAGATAGACTTCAC | 110 | 30.30% | PPMO8.27 |
| H8A(+120+139) | | | | |
| UMOD | TGTCACAGAGATAGACTTCA | 111 | 45.20% | PPMO8.28 |
| H8A(+121+140) | | | | |
| UMOD | GTGTCACAGAGATAGACTTC | 112 | 48.40% | PPMO8.29 |
| H8A(+122+141) | | | | |
| UMOD | TCATTCATGGTGTCACAGAGATAGA | 113 | 46.10% | PMO8.36 |
| H8A(+126+150) | | | | |
| UMOD | TTCATGGTGTCACAGAGATA | 114 | 59.90% | PPMO8.30 |
| H8A(+128+147) | | | | |
| UMOD | ATTCATGGTGTCACAGAGAT | 115 | 53.30% | PPMO8.31 |
| H8A(+129+148) | | | | |
| UMOD | TTTCATTCATGGTGTCACAG | 116 | 0.00% | PPMO8.32 |
| H8A(+133+152) | | | | |
| UMOD | TTTTCATTCATGGTGTCACA | 117 | 0.00% | PPMO8.33 |
| H8A(+134+153) | | | | |
| UMOD | TGCACTTTTCATTCATGGTG | 118 | 0.00% | PPMO8.34 |
| H8A(+139+158) | | | | |
| UMOD | AGGCTTGCACTTTTCATTCA | 119 | 0.00% | PMO8.35 |
| H8D(+19−1) | | | | |
| UMOD | GAGTCAACTCACAGGCTTGCACTTT | 120 | 46.40% | PMO8.37 |
| H8D(+12−13) | | | | |
| UMOD | AATCTGGTCCCAGAGCAGGTCTACA | 121 | 3.90% | PMO9.1 |
| H9A(−5+20) | | | | |
| UMOD | TTCGGAATCTGGTCCCAGAGCAGGT | 122 | 7.20% | PMO9.2 |
| H9A(+1+25) | | | | |
| UMOD | TGTGATGGGACCCAAGTTCAGGACA | 123 | 5.10% | PMO9.3 |
| H9A(+51+75) | | | | |
| UMOD | AGCGAGTGGCTCTCTTACCTTTCCG | 124 | 16.20% | PMO9.4 |
| H9D(+7−18) | | | | |

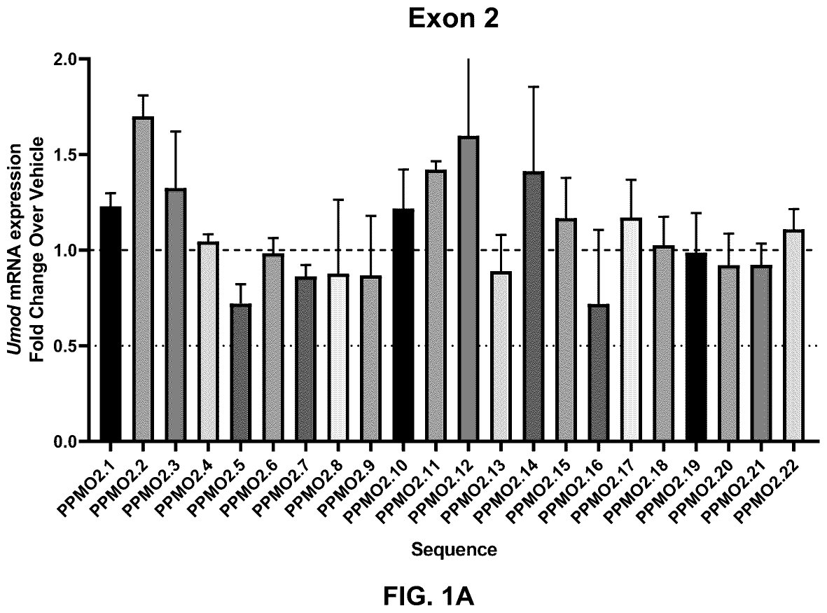

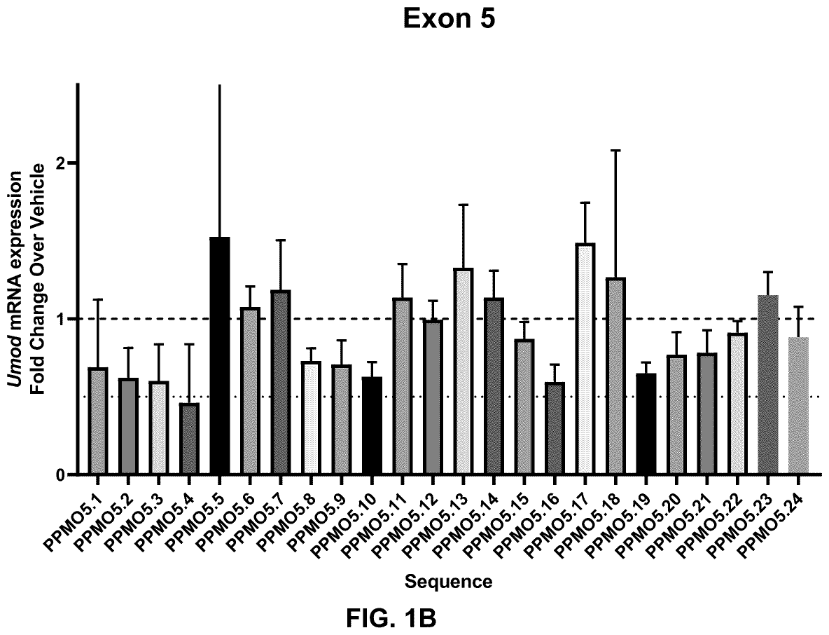

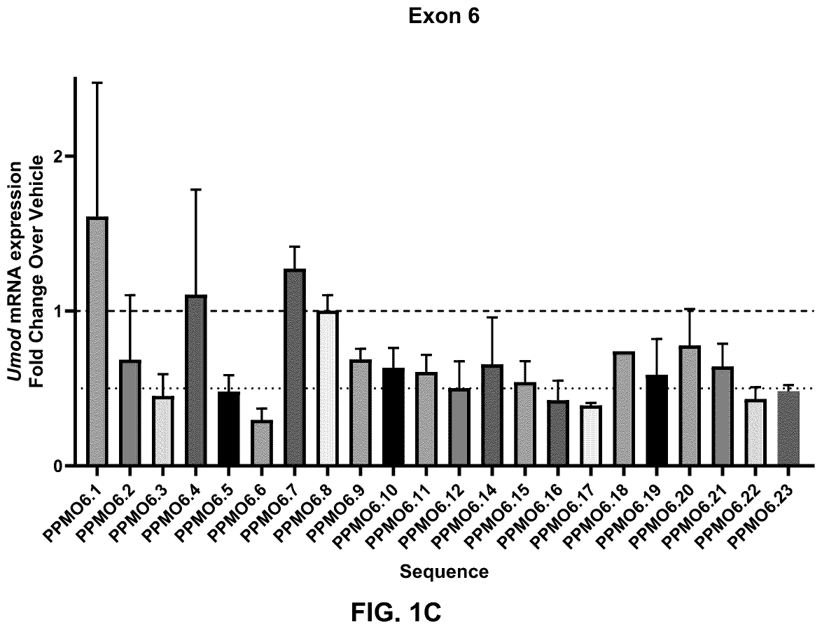

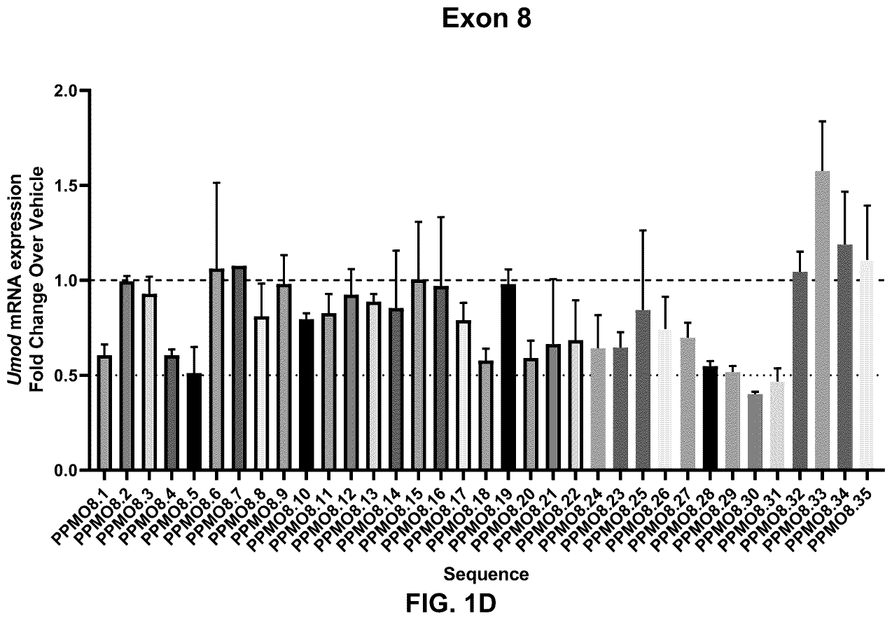

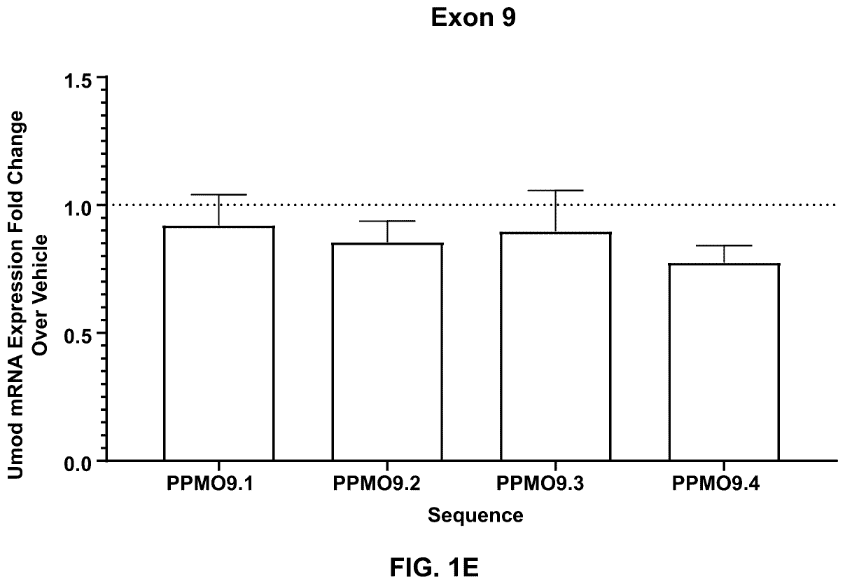

FIGS. 1A-1E show the expression of UMOD mRNA in DMS545 cells transfected with PPMOs of the instant disclosure. In FIGS. 1A-1E, the decrease in UMOD expression is shown compared to the vehicle. Accordingly, a 1-fold change over vehicle represents expression that is equivalent to expression in the vehicle. The most active PPMO compounds exhibited around 0.5-fold expression, e.g., the UMOD expression was reduced to about half the amount of the UMOD transcript. mIMCD-3 cells were seeded in 96 well plates one day before transfection. Cells were transfected with 20 μM of PPMO. Cells were collected after 72-hour incubation. RT-PCR was performed and the % knockdown was calculated to be the percentage of total UMOD transcript less than the vehicle control, after normalization to the housekeeper, TBP. The results are shown in FIG. 1E. The PPMOs used in this example are of structural Formula (IV), having the sequences indicated in Table 5 (i.e., PMO9.1, PMO9.2, PMO9.3, and PMO9.4).

### Example 3—Knockdown of UMOD by PPMO in mIMCD-3 Cells

Mouse inner medullary collecting duct (mIMCD-3) cells, which express endogenous UMOD were seeded in 96 well plates one day before transfection. Cells were transfected with seven different PPMOs (i.e., PPMO1, PPMO2, PPMO3, PPMO4, PPMO5, PPMO6, and PPMO7) at two dosages: 10 μM and 20 μM of PPMO combined with 1 mM endoporter. Additionally, cells were transfected with three different PPMOs (i.e., PPMO1, PPMO2, PPMO3) at four dosages: 2.5 M, 5 μM, 10 μM, and 20 μM of PPMO combined with 1 mM endoporter. After a 72-hour incubation period, cells were collected and analyzed. Real time PCR (RT-PCR) was performed on the transfected mIMCD cells, and the % knockdown was calculated to be percentage of total UMOD transcript less than the vehicle control, after normalization to a housekeeper, TBP. Table 6 and FIGS. 2A and 2B show the UMOD knockdown data in mIMCD-3 cells. The PPMOs used in this example are of Formula (IV) having the sequences indicated in Table 6 (i.e., PPMO1, PPMO2, PPMO3, PPMO4, PPMO5, PPMO6, and PPMO7).

TABLE 6
mIMCD Microwalk Data.

| | PPMO | PPMO | % | PPMO |
|---|---|---|---|---|
| Target Region | (5′-3′) | SEQ ID NO | Knockdown | Name |
| UMOD | ACTCTGCCCTCTGTCCTGGAAACAC | 145 | 29.4% | PPMO1 |
| M2A(+1+25) | | | | |
| UMOD | GGTCAAAGGGATCCCCATCCTTTAC | 146 | 73.4% | PPMO2 |
| M2A(+26+50) | | | | |
| UMOD | TTACCATCATTACCAGCAGCATCCA | 147 | 72.9% | PPMO3 |
| M2A(+51+75) | | | | |
| UMOD | CTTGATGTCATTGGCCCCACACTCC | 148 | 65.8% | PPMO4 |
| M5A(+26+50) | | | | |
| UMOD | CTGAAAATGGTCAGGAACAACAGGA | 149 | 64.3% | PPMO5 |
| M5A(-1-25) | | | | |
| UMOD | TGGGAGACATCTGAAAATGGTCAGG | 150 | 55.6% | PPMO6 |
| M5A(-15+10) | | | | |
| UMOD | TGGTTGTATCTTCTGTACGTGGACAA | 151 | 87.0% | PPMO7 |
| M8A(+1+25) | | | | |
| UMOD | GACTCGCCATTCTCTGTCACCTGAA | 152 | 38.1% | PPMO8 |
| M8A(+26+50) | | | | |
| UMOD | TTCGAAATCTAGTACCAGAGCAGGT | 153 | 70.4% | PPMO9 |
| M9A(+1+25) | | | | |

*The antisense oligomers were prepared with a 5′ terminus capping group of TEG and a 3′ terminus capping group of -G-R₆, where G is glycine and R is arginine.

### Example 4—PPMO Distribution in Kidney

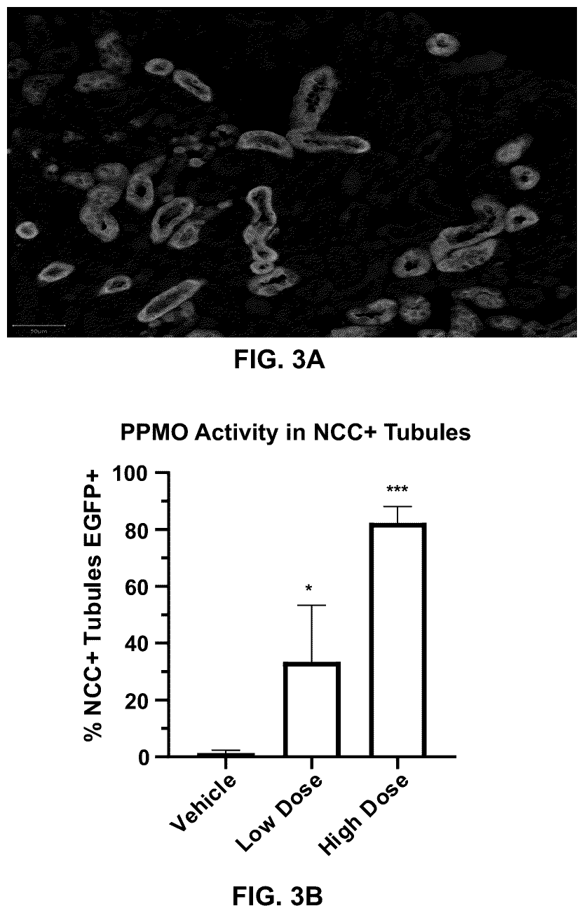

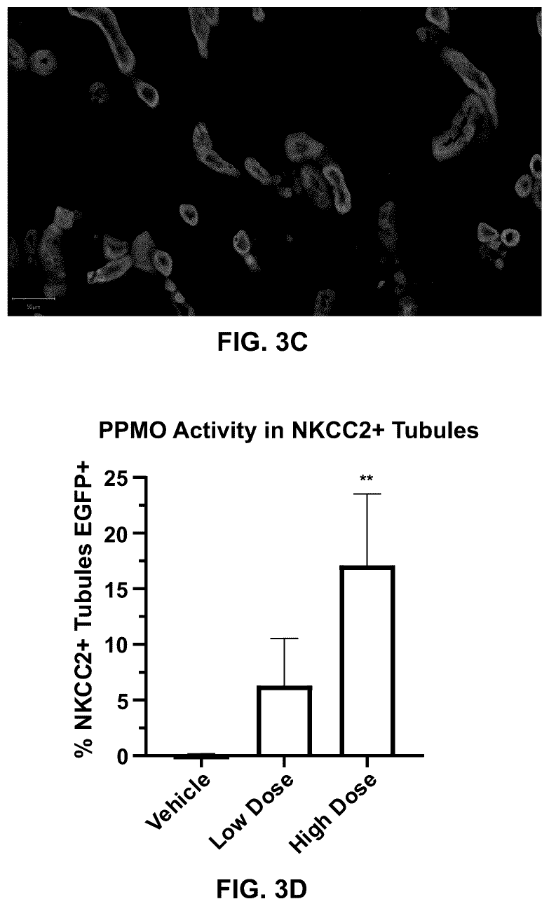

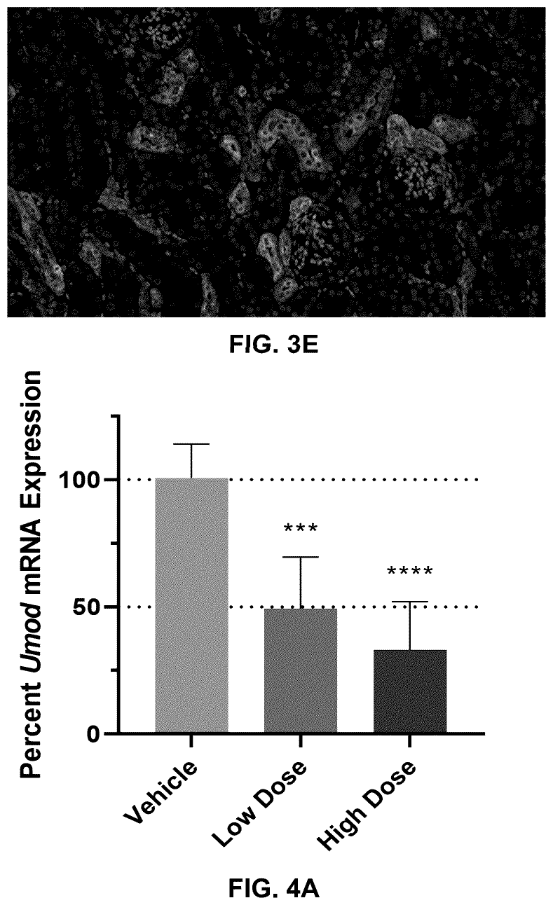

EGFP-654 transgenic mice were injected intravenously with a PPMO targeting the EGFP coding sequence to determine PPMO activity in the kidney (see below for PPMO description). Two doses were tested: 10 mg/kg for low dose and at 80 mg/kg for high dose. Histological sections were co-stained with NCC or NKCC2 and demonstrated that the antisense PPMOs have activity in distal tubular epithelial (FIGS. 3A and 3B) and TAL cells, respectively (FIGS. 3C and 3D). In addition, co-staining with UMOD demonstrated that antisense PPMOs have activity in UMOD expressing cells (FIG. 3E). The PPMO used in this example is of Formula (IV):

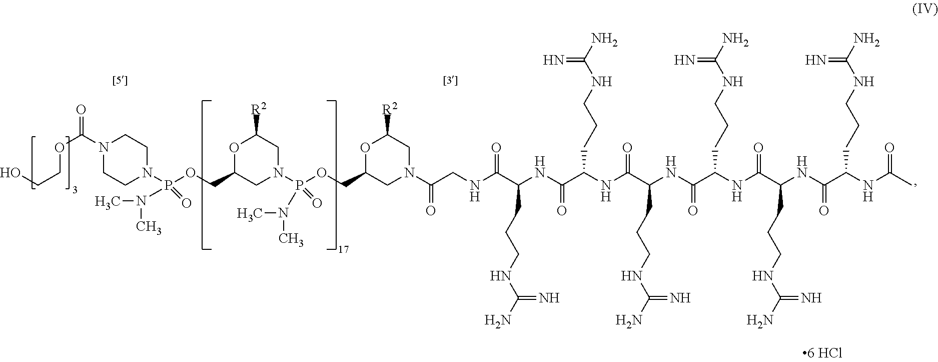

having a base sequence of 5′-GCTATTACCTTAACCCAG-3′ (SEQ ID NO: 135).

### Example 5—In Vivo Proof of Concept

Wild-type mice were injected (IV) with an antisense oligomer of the present disclosure as a proof of concept to demonstrate the efficacy of the oligomers in vivo. Two doses were tested: 30 mg/kg for low dose and at 100 mg/kg for high dose.

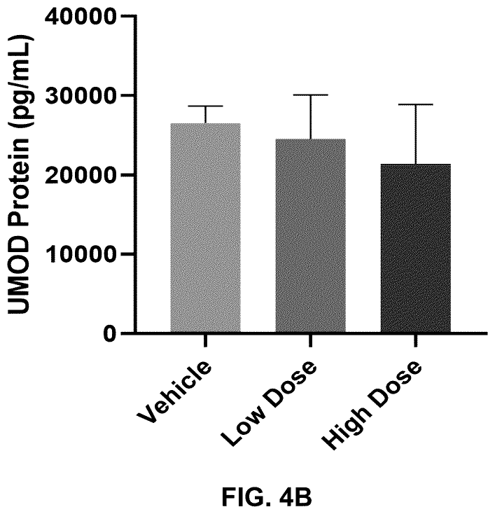

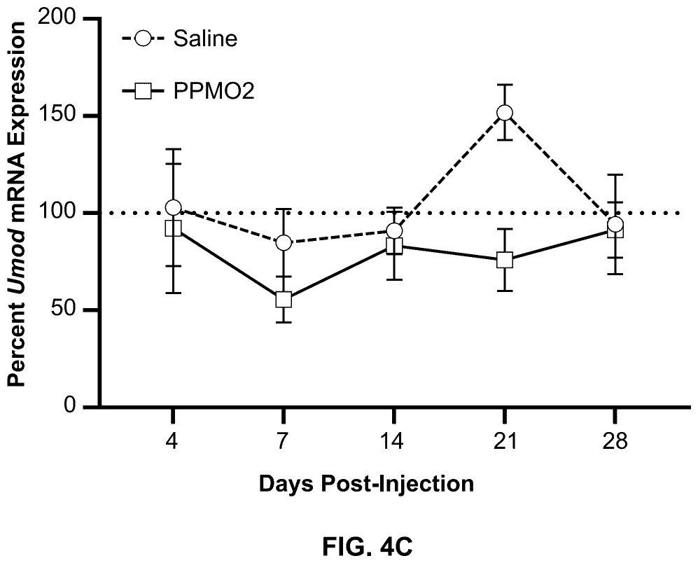

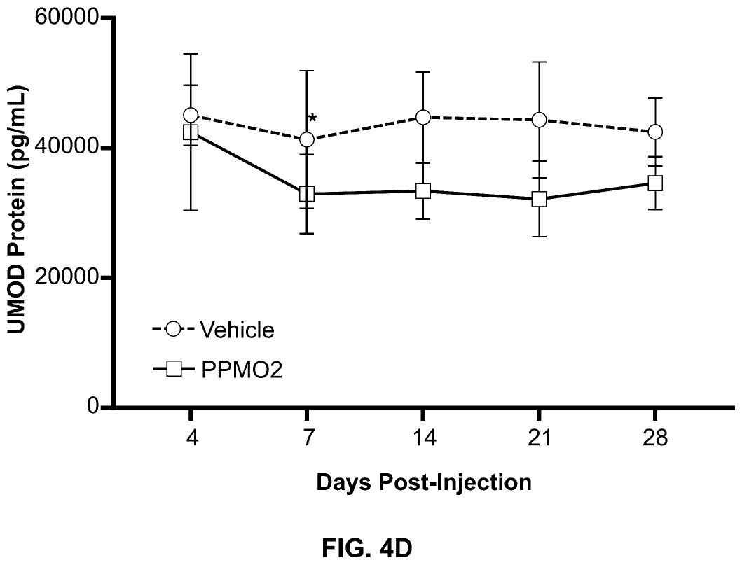

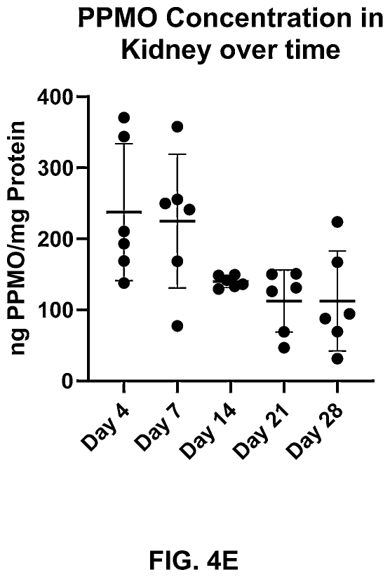

A low or high dose of PPMO2 (e.g., an antisense oligomer of Formula (IV)) was intravenously injected into wild-type mice and the animals were taken down seven days post injection. The percent change in mRNA expression over saline treatment and UMOD protein levels are shown in FIGS. 4A and 4B, respectively. A statistically significant knockdown of UMOD was observed at both doses (p value <0.0001 for high dose; p value <0.005 for low dose).

In another experiment, wild-type mice were injected with either saline or a low dose of PPMO2 and taken down at 4-, 7-, 14-, 21-, and 28-days post injection. Sustained reduction in UMOD protein and mRNA expression was observed, as shown in FIGS. 4C and 4D, respectively. The concentration of PPMO was measured at each time point in FIG. 4E.

### Example 6—UMOD^{C93F} Disease Model

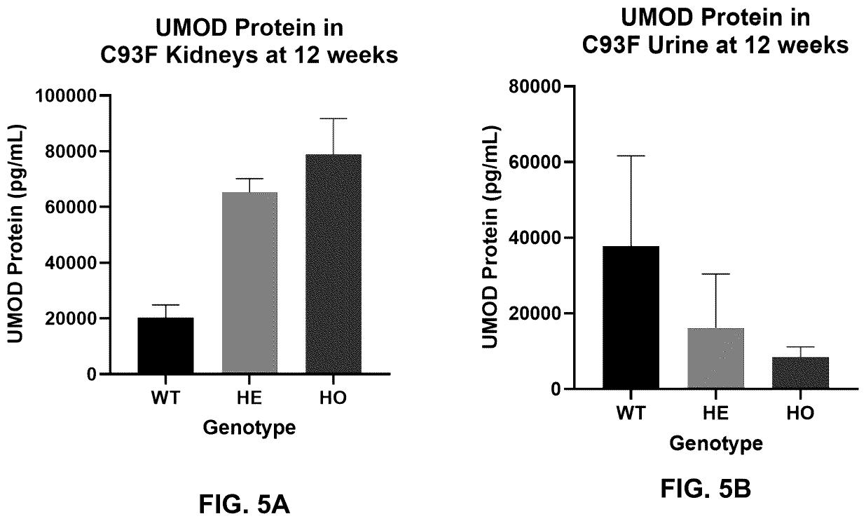

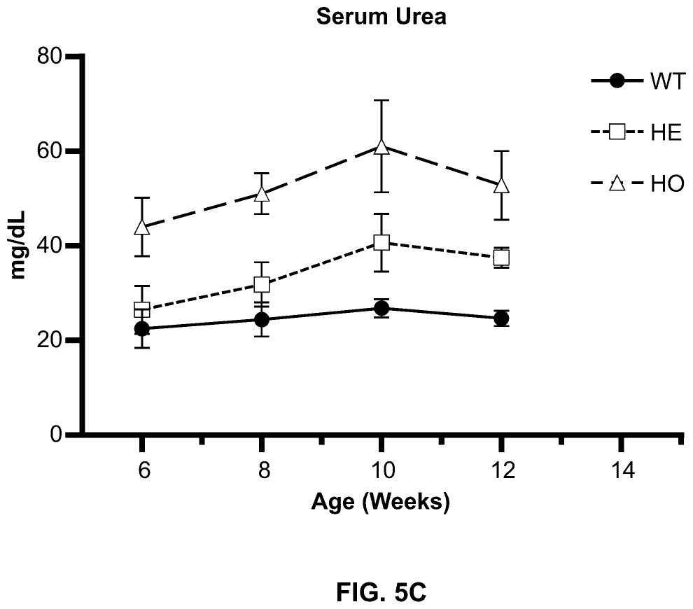

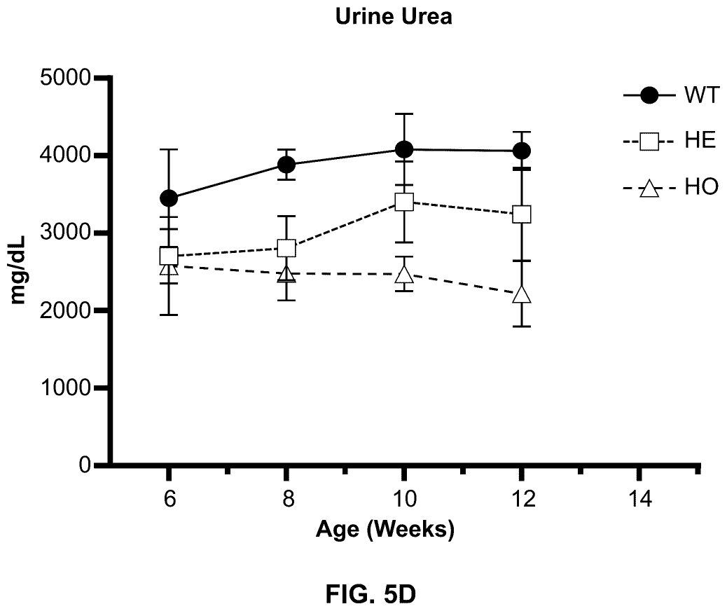

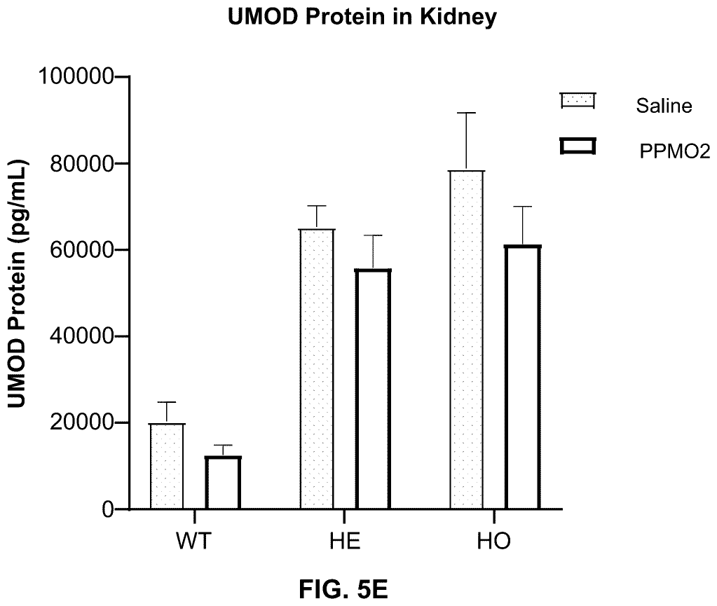

UMOD^{C93F} mouse line was generated by ENU mutagenesis at Helmholtz Zentrum München and is an established disease model of ADTKD (i.e., models ADTKD phenotype in humans). Specifically, the C₉₃F mutation disrupts a disulfide bond and leads to phenotype similar to human ADTKD leading to UMOD aggregation in TAL cells, increased plasma urea, and Interstitial fibrosis and tubular atrophy. To better understand disease progression in UMOD^{C93F}/wt (HE) and UMOD^{C93F/C93F} (HO) animals, UMOD protein expression in the kidney (FIG. 5A) and urine (FIG. 5B) and urea levels (FIGS. 5C and 5D) was studied. PPMO2 at a dose of 100 mg/kg (e.g., an antisense oligomer of Formula (IV)) was injected into WT, HE or HO animals and taken down at 7 days post injection. % Knockdown of UMOD protein is shown in FIG. 5E.

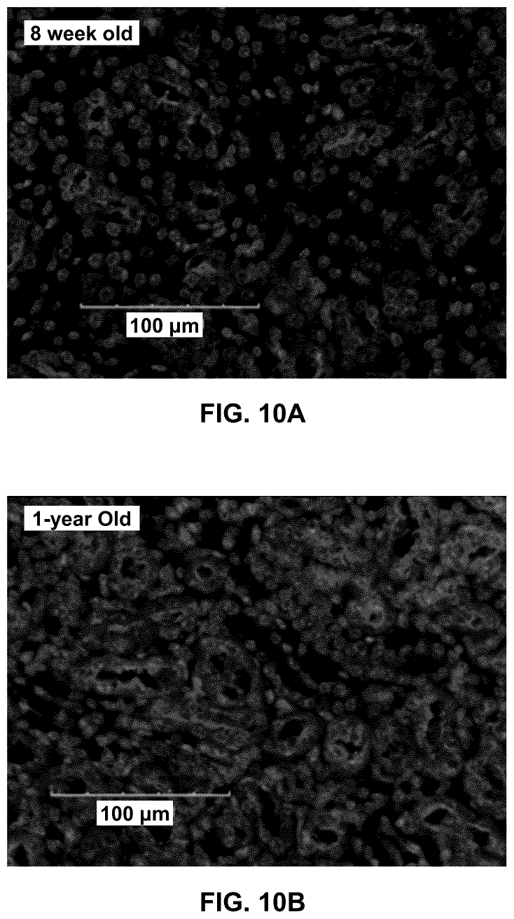

Antisense oligomers described herein were delivered to the kidney and demonstrated activity in kidney cell types relevant to ADTKD-UMOD, including TAL cells and distal convoluted tubule cells. Further, the ability of the disclosed antisense oligomers to reduce UMOD mRNA expression was demonstrated both in vitro and in vivo. In vitro mice studies demonstrated a greater than 85% knockdown of UMOD expression following treatment with an antisense oligomer of the present disclosure. Notably, wild-type mice exhibited a greater than 65% knockdown of UMOD mRNA expression seven days after a single IV injection of a UMOD-targeting antisense oligomer, and a 25% knockdown of UMOD protein 14 days after a single IV injection of a UMOD-targeting antisense oligomer. Untreated kidneys from 8-week-old and one year old UMOD^{C93F} animals were fixed, embedded in paraffin and sectioned. The slides were then stained for Umod expression FIGS. 10A and 10B show the results for 8-week-old treated and one year old untreated UMOD^{C93F} mice, respectively.

### Example 7—EGFP Staining of EGFP-654 Mice

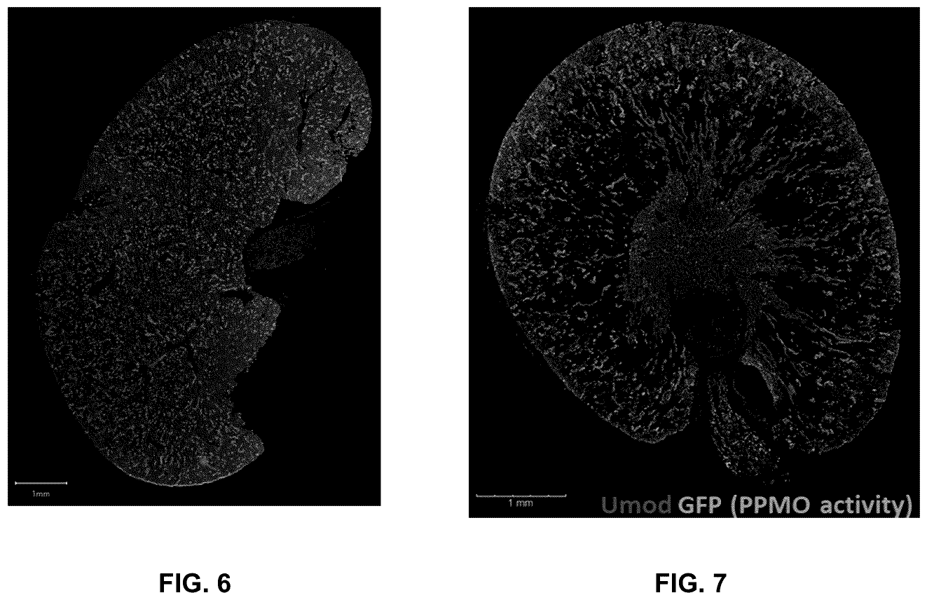

EGFP-654 mice were intravenously injected with 80 mg/kg of PPMO and the animals were taken down 7 days post injection. The kidneys were fixed, and paraffin embedded. The kidneys were sectioned, and the sections were co-stained with EGFP Expression (FIG. 6). The PPMO used in this example is of Formula (IV):

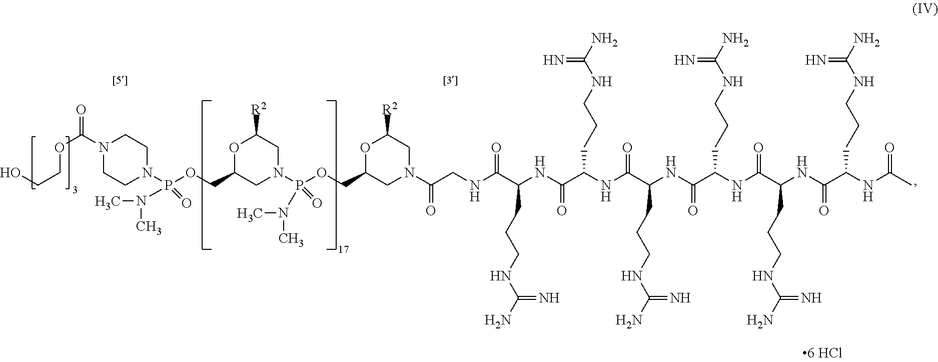

having a base sequence of 5′-GCTATTACCTTAACCCAG-3′ (SEQ ID NO: 135).

### Example 8—EGFP and Umod Co-Staining of EGFP-654 Mice

EGFP-654 mice were treated, with PPMO at a dose of 80 mg/kg and the kidney samples prepared, as described in Example 8. The kidneys were sectioned, and the sections were co-stained with EGFP Expression and Umod (FIG. 7).

### Example 9—Knockdown of UMOD by PPMO in C57BL/6 Mice

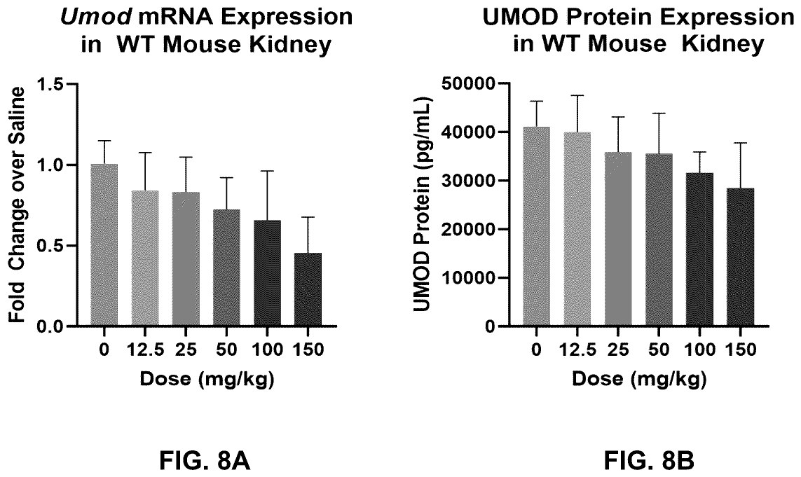

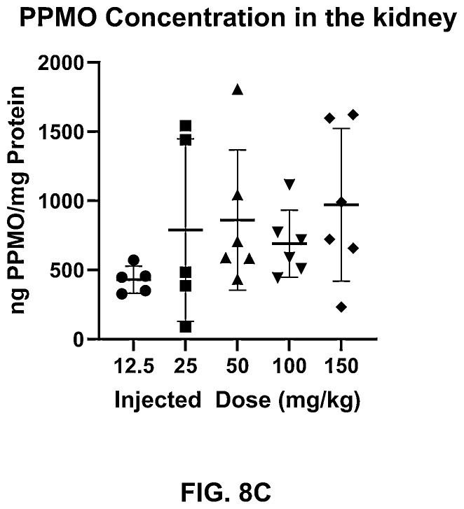

C57BI/6 mice were intravenously injected with different concentrations of PPMO2 and the animals were taken down 7 days post injection. PPMO2 was injected at different doses: 0 (saline), 12.5, 25, 50, 100, and 150 mg/kg. The percent change in mRNA expression over saline treatment and UMOD protein levels are shown in FIG. 8A and FIG. 8B. The concentration of PPMO in the kidney at each time point is shown in FIG. 8C.

### Example 10—Knockdown of UMOD by PPMO in C57BL/6 Mice

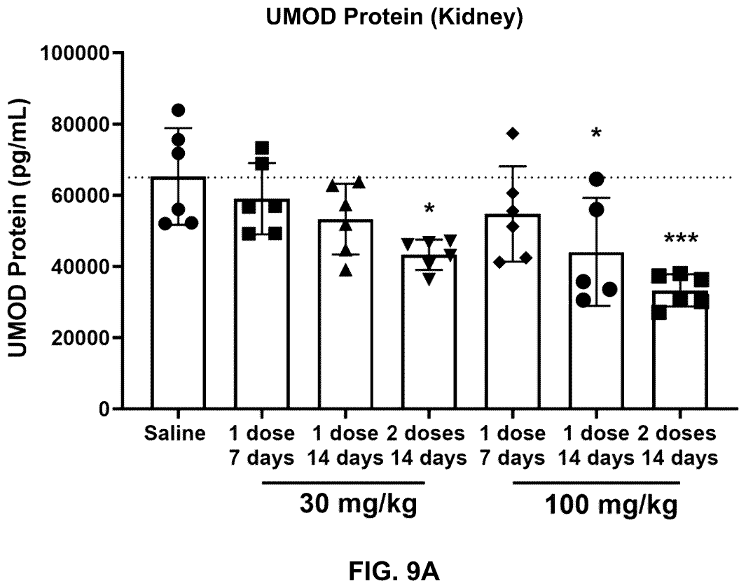

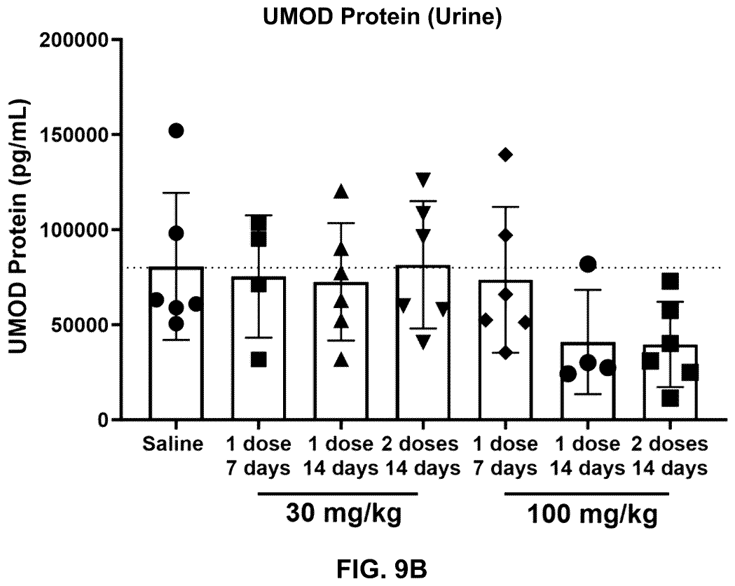

C57BI/6 mice were intravenously injected with either 30 mg/kg or 100 mg/kg PPMO2, once or twice at one week apart, and the animals were taken down 7- or 14-days post injection. The total UMOD protein in the kidney and urine are shown in FIGS. 9A and 9B, respectively.

### Example 11—Knockdown of UMOD by PPMO in Heterozygous Umod C93F Animals

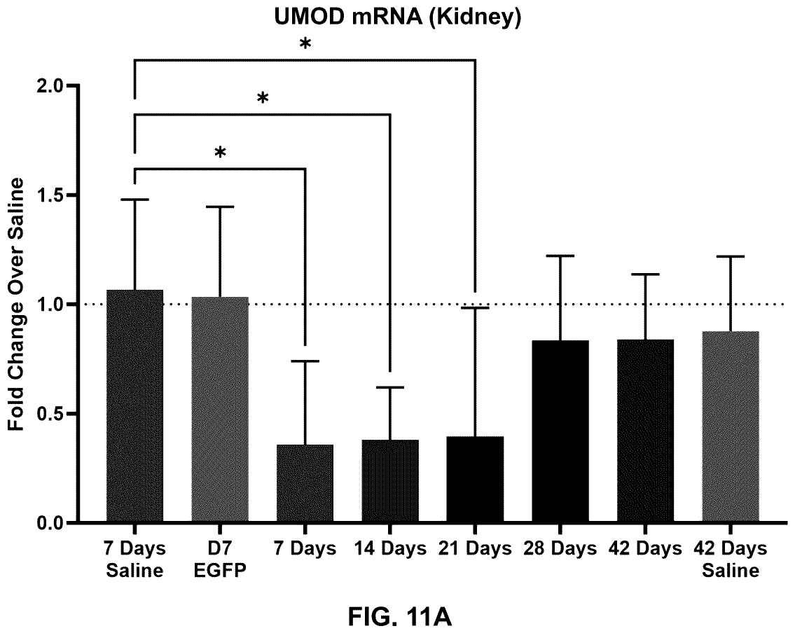

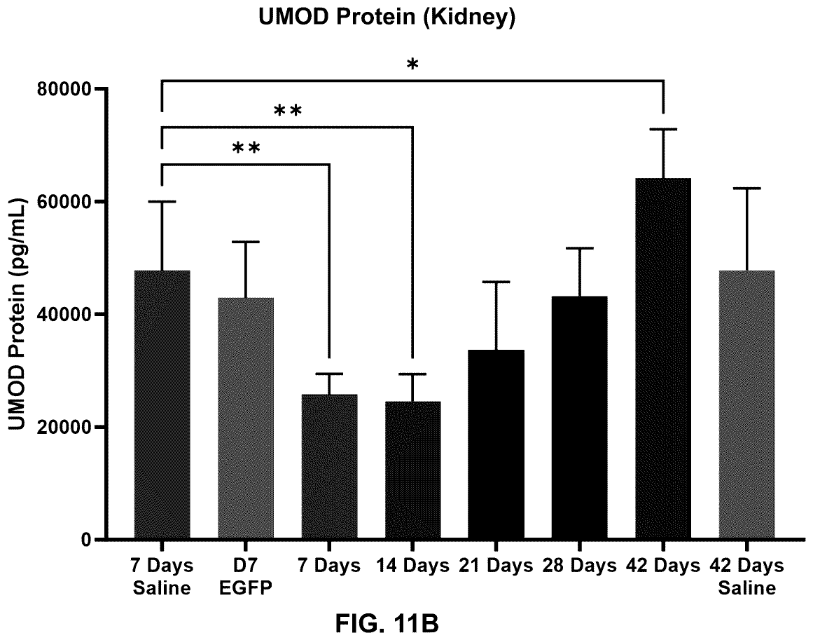

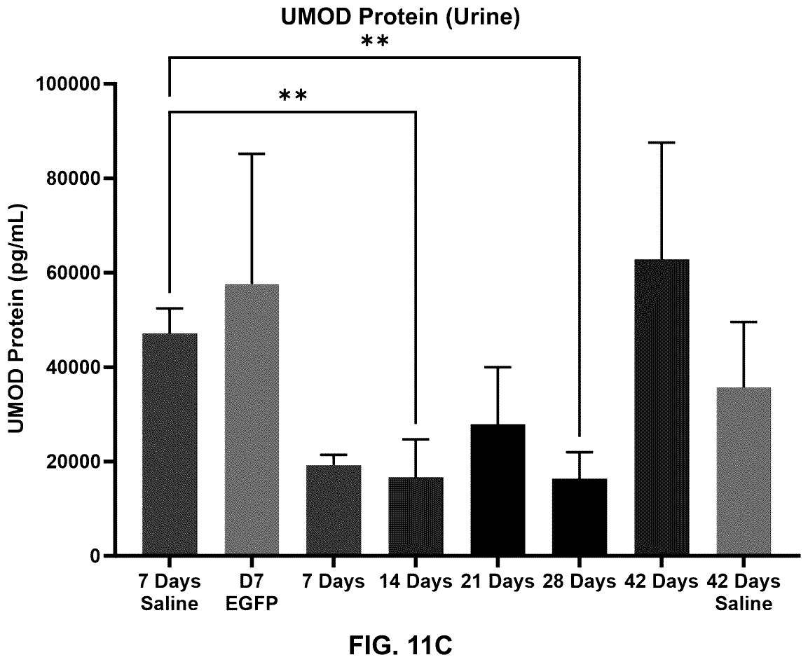

Heterozygous Umod C93F mice were intravenously injected with 100 mg/kg of PPMO2 and the animals were taken down 7-, 14-, 21-, 28- or 42-days post injection. The percent change in mRNA expression over saline treatment and UMOD protein levels in both kidney and urine are shown in FIG. 11A (mRNA expression over saline treatment), FIG. 11B (UMOD protein levels in kidney), and FIG. 11C (UMOD protein levels in urine). FIGS. 11A-11C demonstrate target engagement in ADTKD-UMOD disease model. Single doses in UMOD^{C93F} mice result in robust and durable UMOD knockdown.

### Example 12—Knockdown of UMOD by PPMO in Heterozygous Umod C₉₃F Animals

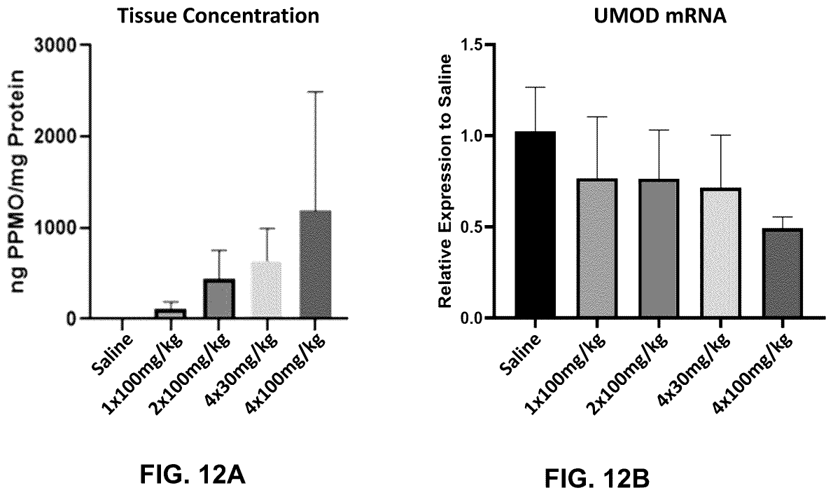

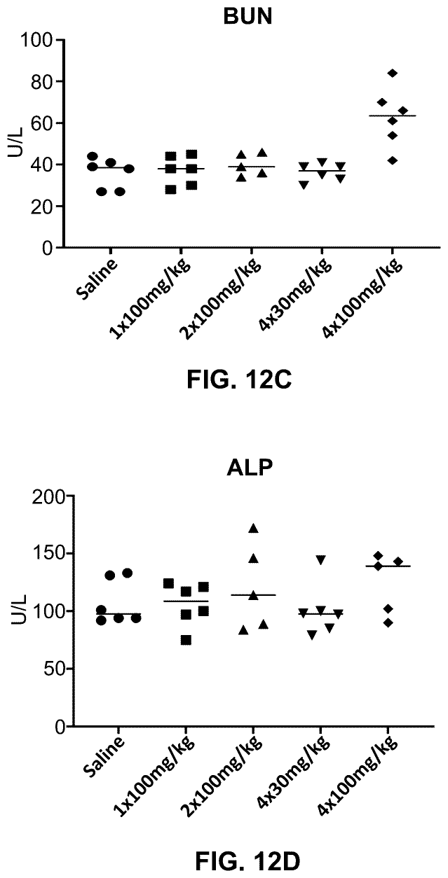

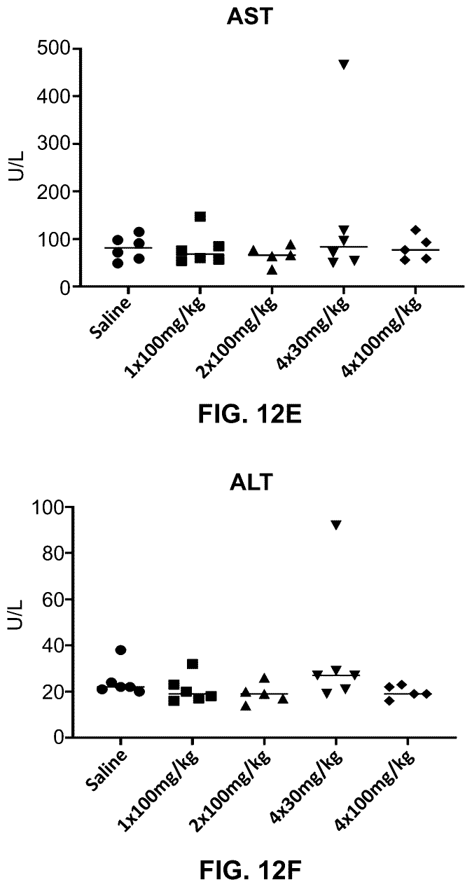

Heterozygous Umod C93F mice were intravenously injected once, twice, or four times at 30 mg/kg or 100 mg/kg PPMO2 and taken down after 4 weeks. The amount of PPMO2 in the kidney and the UMOD mRNA in the kidney are shown in FIGS. 12A and 12B, respectively demonstrating a dose-dependent increase in tissue compound concentration, and UMOD knockdown in UMOD^{C93F} mice. Blood chemistry panel data is shown in FIGS. 12C-12F demonstrating that multiple PPMO dosing is well tolerated,

### Example 13—PPMO Splicing Specificity

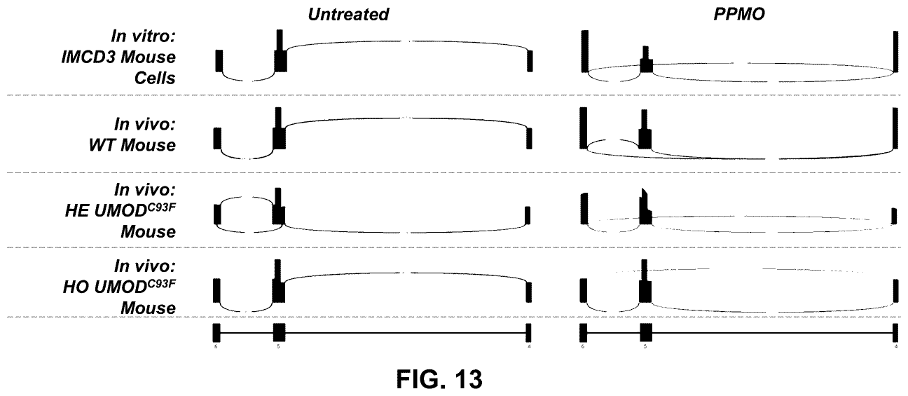

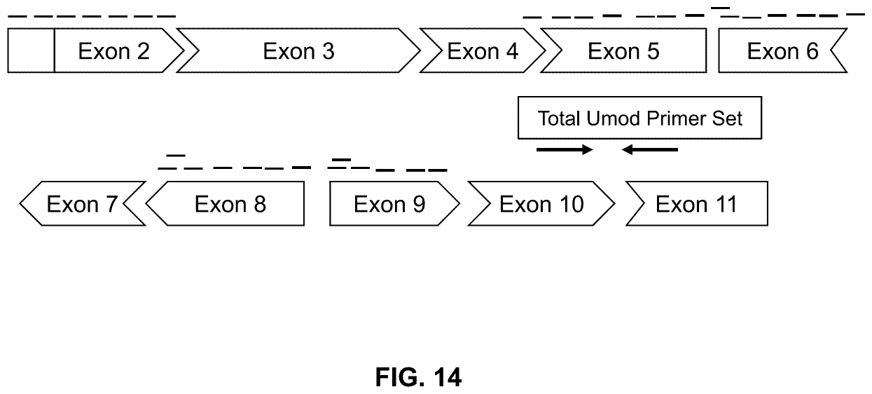

RNA from cells or animals treated with an exon 5 skipping PPMO was sequenced to show that the expected exon skipping is observed. Imcd3 cells were treated with PPMO2 for 3 days. WT, heterozygous, or homozygous Umod C93F mice were intravenously injected with 100 mg/kg of PPMO2 and the kidneys were collected 7 days post-injection. Total RNA was extracted from cells or kidney homogenate and UMOD Ex4-6 PCR amplified. Amplicon pool subjected to amplicon sequencing. Shown junctions representing >5% reads compared to WT exon junction (FIG. 13). A single, out-of-frame exon 4-6 splice product creating a premature termination codon (PTC) was observed (in vitro and in vivo).

### Example 14—Determination of TEER in TALH Cells

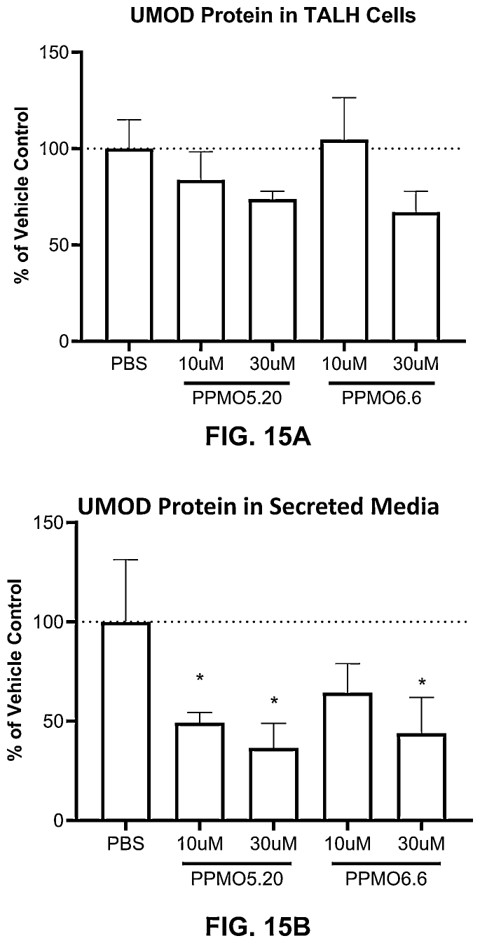

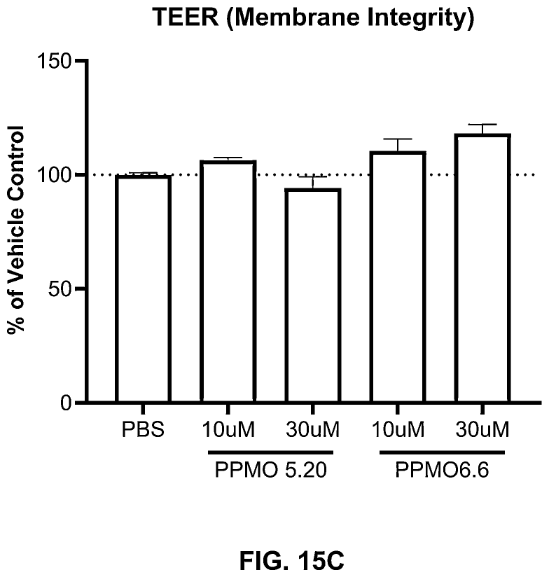

The primary thick ascending limb of the Loop of Henle (TALH) cells were isolated and purified from human kidneys. TALH cells were cultured on transwell plates until confluent. Samples were treated with PPMO5.20 and PPMO6.6, at 10 μM or 30 μM of PPMO1 or PPMO2 and analyzed 48 hours later for UMOD protein levels in the cells and in the media. TEER (measure of electrical resistance across membrane) was determined and showed no deterioration of the membrane after treatment. TALH cells are epithelial cells and form the lining of the loop of Henle. TEER measures the confluency and is an indicator of the cell barrier integrity. Multiple PPMOs were shown to reduce UMOD in primary human TALH cells. Further, PPMO treatment did not impact membrane integrity for human kidney TALH cells (FIGS. 15A-15C).

TABLE 7
Antisense Oligomer Sequences.

| | | SEQ |
|---|---|---|
| Exon Target | Antisense Oligomer Sequences | ID |
| Regions | (5′-3′) | NO. |
| UMOD H2A(−25−1) | CTGTGAACAGAGATGGATGGGACAA | 7 |
| UMOD H2A(−18+2) | TCCTGTGAACAGAGATGGAT | 8 |
| UMOD H2A(−17+3) | GTCCTGTGAACAGAGATGGA | 9 |
| UMOD H2A(−16+4) | TGTCCTGTGAACAGAGATGG | 10 |
| UMOD H2A(−15+10) | GTCTGGTGTCCTGTGAACAGAGATG | 11 |
| UMOD H2A(+1+25) | CTCTCTGTCTCTGATGTCTGGTGTC | 12 |
| UMOD H2A(+26+50) | AGACGGGTTGGCCCTTTGAATTTTT | 13 |
| UMOD H2A(+51+75) | TCTAGATAGCACCTGCCCAAAGGAA | 14 |
| UMOD H2A(+85+104) | ATCCTTTCTGCTCTTCCCGC | 15 |
| UMOD H2A(+86+105) | CATCCTTTCTGCTCTTCCCG | 16 |
| UMOD H2A(+95+119) | AGAGATGGCTGCCCCATCCTTTCTG | 17 |
| UMOD H2A(+96+115) | ATGGCTGCCCCATCCTTTCT | 18 |
| UMOD H2A(+98+117) | AGATGGCTGCCCCATCCTTT | 19 |
| UMOD H2A(+101+125) | CAAGTCAGAGATGGCTGCCCCATCC | 20 |
| UMOD H2A(+108+127) | TCCAAGTCAGAGATGGCTGC | 21 |
| UMOD H2A(+109+128) | ATCCAAGTCAGAGATGGCTG | 22 |
| UMOD H2A(+110+129) | CATCCAAGTCAGAGATGGCT | 23 |
| UMOD H2A(+113+132) | CAGCATCCAAGTCAGAGATG | 24 |
| UMOD H2A(+114+133) | TCAGCATCCAAGTCAGAGAT | 25 |
| UMOD H2A(+118+137) | ACCATCAGCATCCAAGTCAG | 26 |
| UMOD H2A(+126+150) | AGAGGCCACCACCACCATCAGCATC | 27 |
| UMOD H2A(+151+175) | CAGTGGCTGCAGTTGTGATGAACCA | 28 |
| UMOD H2D(+15−10) | TTTCACTTACTTGCTTCTGAGGTGT | 29 |
| UMOD H5A(−15+5) | GATATCTGAAACAGGTTAGG | 30 |
| UMOD H5A(−14+6) | AGATATCTGAAACAGGTTAG | 31 |
| UMOD H5A(−11+9) | GGGAGATATCTGAAACAGGT | 32 |
| UMOD H5A(−10+10) | AGGGAGATATCTGAAACAGG | 33 |
| UMOD H5A(−9+11) | GAGGGAGATATCTGAAACAG | 125 |
| UMOD H5A(+50+67) | CACTTGCCCAGCGACACC | 34 |
| UMOD H5A(+51+75) | TCAGCTGGCACTTGCCCAGCGACAC | 35 |
| UMOD H5A(+52+69) | GGCACTTGCCCAGCGACA | 36 |
| UMOD H5A(+76+100) | AAGACCTTGTCGAAGCCCAGACTCT | 37 |
| UMOD H5A(+78+95) | CTTGTCGAAGCCCAGACT | 38 |
| UMOD H5A(+80+97) | ACCTTGTCGAAGCCCAGA | 39 |
| UMOD H5A(+82+99) | AGACCTTGTCGAAGCCCA | 40 |
| UMOD H5A(+85+102) | TGAAGACCTTGTCGAAGC | 41 |
| UMOD H5A(+86+103) | ATGAAGACCTTGTCGAAG | 42 |
| UMOD H5A(+91+108) | GGTACATGAAGACCTTGT | 43 |
| UMOD H5A(+126+150) | GGTTGTCTCTGTCATTGAAGCCCGA | 44 |
| UMOD H5A(+152+171) | TCACTACAGACACCCAGTCC | 45 |
| UMOD H5A(+153+172) | GTCACTACAGACACCCAGTC | 46 |
| UMOD H5A(+154+173) | GGTCACTACAGACACCCAGT | 47 |
| UMOD H5D(+18−2) | ACCGTCAACACTGTCCCACA | 48 |
| UMOD H5D(+17−3) | TACCGTCAACACTGTCCCAC | 49 |
| UMOD H5D(+16−4) | GTACCGTCAACACTGTCCCA | 50 |
| UMOD H5D(+14−6) | ACGTACCGTCAACACTGTCC | 51 |
| UMOD H5D(+13−7) | GACGTACCGTCAACACTGTC | 52 |
| UMOD H5D(+12−8) | GGACGTACCGTCAACACTGT | 53 |
| UMOD H5D(+11−9) | AGGACGTACCGTCAACACTG | 54 |
| UMOD H5D(+10−10) | CAGGACGTACCGTCAACACT | 55 |
| UMOD H6A(+1+25) | TGTAAGTGGCATGGGTTTCATTCCT | 56 |
| UMOD H6A(+26+50) | TCATCTGCCAGGTAGAGGGTGTTGC | 57 |
| UMOD H6A(+48+67) | GGTCACGGATGATGATCTCA | 58 |
| UMOD H6A(+49+68) | AGGTCACGGATGATGATCTC | 59 |
| UMOD H6A(+50+69) | GAGGTCACGGATGATGATCT | 60 |
| UMOD H6A(+58+77) | TTGATGTTGAGGTCACGGAT | 61 |
| UMOD H6A(+59+78) | TTTGATGTTGAGGTCACGGA | 62 |
| UMOD H6A(+60+79) | TTTTGATGTTGAGGTCACGG | 63 |
| UMOD H6A(+76+100) | GGTAGGAGCATGCAAAGTTGATTTT | 64 |
| UMOD H6A(+79+98) | TAGGAGCATGCAAAGTTGAT | 65 |
| UMOD H6A(+101+125) | TTCAGGCTGACTTTCATGTCCAGGG | 66 |
| UMOD H6A(+101+120) | GCTGACTTTCATGTCCAGGG | 67 |
| UMOD H6A(+110+129) | GGTCTTCAGGCTGACTTTCA | 68 |
| UMOD H6A(+111+130) | CGGTCTTCAGGCTGACTTTC | 69 |
| UMOD H6A(+112+131) | GCGGTCTTCAGGCTGACTTT | 70 |
| UMOD H6A(+113+132) | GGCGGTCTTCAGGCTGACTT | 71 |
| UMOD H6A(+119+138) | CTGTAGGGCGGTCTTCAGGC | 72 |
| UMOD H6A(+120+139) | GCTGTAGGGCGGTCTTCAGG | 73 |
| UMOD H6A(+121+140) | GGCTGTAGGGCGGTCTTCAG | 74 |
| UMOD H6A(+122+141) | TGGCTGTAGGGCGGTCTTCA | 75 |
| UMOD H6A(+123+142) | TTGGCTGTAGGGCGGTCTTC | 76 |
| UMOD H6A(+124+143) | ATTGGCTGTAGGGCGGTCTT | 77 |
| UMOD H6A(+130+149) | CTGACCATTGGCTGTAGGGC | 78 |
| UMOD H6D(+24−1) | CCTGACCATTGGCTGTAGGGCGGTC | 79 |
| UMOD H6D(+15−5) | CACACCTGACCATTGGCTGT | 80 |
| UMOD H6D(+14−6) | CCACACCTGACCATTGGCTG | 81 |
| UMOD H8A(−2+23) | TTGAGTCTCTAGTGTGTGGGCATCT | 82 |
| UMOD H8A(+26+50) | ACTCCCCATTCTCCACCACTTGGAT | 83 |
| UMOD H8A(+51+75) | TGGACGGAAAATCGGCCCTGGGAGG | 84 |
| UMOD H8A(+60+79) | CATCTGGACGGAAAATCGGC | 85 |
| UMOD H8A(+61+80) | ACATCTGGACGGAAAATCGG | 86 |
| UMOD H8A(+62+81) | AACATCTGGACGGAAAATCG | 87 |
| UMOD H8A(+63+82) | GAACATCTGGACGGAAAATC | 88 |
| UMOD H8A(+68+87) | AACCGGAACATCTGGACGGA | 89 |
| UMOD H8A(+69+88) | AAACCGGAACATCTGGACGG | 90 |
| UMOD H8A(+70+89) | CAAACCGGAACATCTGGACG | 91 |
| UMOD H8A(+76+95) | TTCCAGCAAACCGGAACATC | 92 |
| UMOD H8A(+76+100) | ATAGTTTCCAGCAAACCGGAACATC | 93 |
| UMOD H8A(+77+96) | TTTCCAGCAAACCGGAACAT | 94 |
| UMOD H8A(+78+97) | GTTTCCAGCAAACCGGAACA | 95 |
| UMOD H8A(+79+98) | AGTTTCCAGCAAACCGGAAC | 96 |
| UMOD H8A(+80+99) | TAGTTTCCAGCAAACCGGAA | 97 |
| UMOD H8A(+81+100) | ATAGTTTCCAGCAAACCGGA | 98 |
| UMOD H8A(+82+101) | CATAGTTTCCAGCAAACCGG | 99 |
| UMOD H8A(+83+102) | TCATAGTTTCCAGCAAACCG | 100 |
| UMOD H8A(+86+105) | AGGTCATAGTTTCCAGCAAA | 101 |
| UMOD H8A(+94+113) | GGTAGACTAGGTCATAGTTT | 102 |
| UMOD H8A(+95+114) | AGGTAGACTAGGTCATAGTT | 103 |
| UMOD H8A(+96+115) | CAGGTAGACTAGGTCATAGT | 104 |
| UMOD H8A(+101+125) | CTTCACAGTGCAGGTAGACTAGGTC | 105 |
| UMOD H8A(+102+121) | ACAGTGCAGGTAGACTAGGT | 106 |
| UMOD H8A(+103+122) | CACAGTGCAGGTAGACTAGG | 107 |
| UMOD H8A(+104+123) | TCACAGTGCAGGTAGACTAG | 108 |
| UMOD H8A(+105+124) | TTCACAGTGCAGGTAGACTA | 109 |
| UMOD H8A(+120+139) | GTCACAGAGATAGACTTCAC | 110 |
| UMOD H8A(+121+140) | TGTCACAGAGATAGACTTCA | 111 |
| UMOD H8A(+122+141) | GTGTCACAGAGATAGACTTC | 112 |
| UMOD H8A(+126+150) | TCATTCATGGTGTCACAGAGATAGA | 113 |
| UMOD H8A(+128+147) | TTCATGGTGTCACAGAGATA | 114 |
| UMOD H8A(+129+148) | ATTCATGGTGTCACAGAGAT | 115 |
| UMOD H8A(+133+152) | TTTCATTCATGGTGTCACAG | 116 |
| UMOD H8A(+134+153) | TTTTCATTCATGGTGTCACA | 117 |
| UMOD H8A(+139+158) | TGCACTTTTCATTCATGGTG | 118 |
| UMOD H8D(+19−1) | AGGCTTGCACTTTTCATTCA | 119 |
| UMOD H8D(+12−13) | GAGTCAACTCACAGGCTTGCACTTT | 120 |
| UMOD H9A(−5+20) | AATCTGGTCCCAGAGCAGGTCTACA | 121 |
| UMOD H9A(+1+25) | TTCGGAATCTGGTCCCAGAGCAGGT | 122 |
| UMOD H9A(+51+75) | TGTGATGGGACCCAAGTTCAGGACA | 123 |
| UMOD H9D(+7−18) | AGCGAGTGGCTCTCTTACCTTTCCG | 124 |
| UMOD M2A(+1+25) | ACTCTGCCCTCTGTCCTGGAAACAC | 126 |
| UMOD M2A(+26+50) | GGTCAAAGGGATCCCCATCCTTTAC | 127 |
| UMOD M2A(+51+75) | TTACCATCATTACCAGCAGCATCCA | 128 |
| UMOD M5A(+26+50) | CTTGATGTCATTGGCCCCACACTCC | 129 |
| UMOD M5A(−1−25) | CTGAAAATGGTCAGGAACAACAGGA | 130 |
| UMOD M5A(−15+10) | TGGGAGACATCTGAAAATGGTCAGG | 131 |
| UMOD M8A(+1+25) | TGGTTGTATCTTCTGTACGTGGACAA | 132 |
| UMOD M8A(+26+50) | GACTCGCCATTCTCTGTCACCTGAA | 133 |
| UMOD M9A(+1+25) | TTCGAAATCTAGTACCAGAGCAGGT | 134 |

1-199. (canceled)

200. An antisense oligomer, or a pharmaceutically acceptable salt thereof, comprising a non-natural chemical backbone and a targeting sequence of 13 to 30 bases in length that is complementary to a target region within a pre-mRNA of the human uromodulin (UMOD) gene represented by SEQ ID NO: 1, wherein the target region is an intron/exon junction or an exon internal region of the human UMOD gene pre-mRNA.

201. The antisense oligomer, or a pharmaceutically acceptable salt thereof, of claim 200, wherein the target region is an intron/exon junction or exon internal sequence of exon 2 (SEQ ID NO: 2), exon 5 (SEQ ID NO: 3), exon 6 (SEQ ID NO: 4), exon 8 (SEQ ID NO: 5), or exon 9 (SEQ ID NO: 6).

202. The antisense oligomer, or a pharmaceutically acceptable salt thereof, of claim 201, wherein the target region is selected from H2A(−15+10), H2A(+1+25), H2A(+26+50), H2A(+51+75), H2A(+85+104), H2A(+86+105), H2A(+95+119), H2A(+101+125), H2A(+110+129), H2A(+118+137), H2A(+126+150), and H2A(+151+175).

203. The antisense oligomer, or a pharmaceutically acceptable salt thereof, of claim 202, wherein the targeting sequence comprises a sequence selected from SEQ ID NOs: 11-17, 20, 23, and 26-28.

204. The antisense oligomer, or a pharmaceutically acceptable salt thereof, of claim 201, wherein the target region is:
(i) within the 51^{st} nucleotide to the 119^{th} nucleotide from the 5′ end of exon 2 (SEQ ID NO: 2) of the human UMOD gene pre-mRNA;
(ii) within the 51^{st} nucleotide to the 105^{th} nucleotide from the 5′ end of exon 2 (SEQ ID NO: 2) of the human UMOD gene pre-mRNA;
(iii) within the 85^{th} nucleotide to the 119^{th} nucleotide from the 5′ end of exon 2 (SEQ ID NO: 2) of the human UMOD gene pre-mRNA;
(iv) within the 15^{th} nucleotide of intron 1 measured from the 5′ end of exon 2 (SEQ ID NO: 2) to the 25^{th} nucleotide of exon 2 measured from the 5′ end of exon 2 (SEQ ID NO: 2); or
(v) within the 118^{th} nucleotide to the 150^{th} nucleotide of exon 2 as measured from the 5′ end of exon 2 (SEQ ID NO: 2).

205. The antisense oligomer, or a pharmaceutically acceptable salt thereof, of claim 201, wherein the target region is selected from H5A(−15+5), H5A(−14+6), H5A(−11+9), H5A(−10+10), H5A(+51+75), H5A(+76+100), H5A(+78+95), H5A(+80+97), H5A(+82+99), H5A(+126+150), H5A(+153+172), H5A(+154+173), H5D(+18-2), H5D(+16-4), H5D(+14-6), H5D(+13-7), and H5D(+12-8).

206. The antisense oligomer, or a pharmaceutically acceptable salt thereof, of claim 205, wherein the targeting sequence comprises a sequence selected from SEQ ID NOs: 30-33, 35, 37-40, 44, 46-48, and 50-53.

207. The antisense oligomer, or a pharmaceutically acceptable salt thereof, of claim 201, wherein the target region is:
(i) within the 15^{th} nucleotide of intron 4 measured from the 5′ end of exon 5 (SEQ ID NO: 3) to the 10^{th} nucleotide of exon 5 measured from the 5′ end of exon 5 (SEQ ID NO: 3) of the human UMOD gene pre-mRNA;
(ii) within the 153^{rd} nucleotide to the 173^{rd} nucleotide measured from the 5′ end of exon 5 (SEQ ID NO: 3) of the human UMOD gene pre-mRNA; or
(iii) within the 18^{th} nucleotide of exon 5 measured from 3′ end of exon 5 (SEQ ID NO: 3) to the 8^{th} nucleotide of intron 5 measured from 3′ end of exon 5 (SEQ ID NO: 3).

208. The antisense oligomer, or a pharmaceutically acceptable salt thereof, of claim 201, wherein the target region is selected from H6A(+26+50), H6A(+48+67), H6A(+49+68), H6A(+58+77), H6A(+59+78), H6A(+76+100), H6A(+101+125), H6A(+101+120), H6A(+110+129), H6A(+111+130), H6A(+112+131), H6A(+113+132), H6A(+119+138), H6A(+120+139), H6A(+121+140), H6A(+122+141), H6A(+123+142), H6A(+124+143), H6A(+130+149), H6D(+24-1), H6D(+15-5), and H6D(+14-6).

209. The antisense oligomer, or a pharmaceutically acceptable salt thereof, of claim 208, wherein the targeting sequence comprises a sequence selected from SEQ ID NOs: 57-59, 61, 62, 64, and 66-81.

210. The antisense oligomer, or a pharmaceutically acceptable salt thereof, of claim 201, wherein the target region is:
(i) within the 48^{th} nucleotide to the 78^{th} nucleotide measured from the 5′ end of exon 6 (SEQ ID NO: 4) of the human UMOD gene pre-mRNA;
(ii) within the 58^{th} nucleotide to the 78^{th} nucleotide measured from the 5′ end of exon 6 (SEQ ID NO: 4) of the human UMOD gene pre-mRNA;
(iii) within the 101^{st} nucleotide to the 132^{nd} nucleotide measured from the 5′ end of exon 6 (SEQ ID NO: 4) of the human UMOD gene pre-mRNA;
(iv) within the 111^{th} nucleotide to the 132^{nd} nucleotide measured from the 5′ end of exon 6 (SEQ ID NO: 4) of the human UMOD gene pre-mRNA;
(v) within the 119^{th} nucleotide to the 149^{th} nucleotide measured from the 5′ end of exon 6 (SEQ ID NO: 4) of the human UMOD gene pre-mRNA;
(vi) within the 119^{th} nucleotide to the 141^{st} nucleotide measured from the 5′ end of exon 6 (SEQ ID NO: 4) of the human UMOD gene pre-mRNA;
(vii) within the 120^{th} nucleotide to the 141^{st} nucleotide measured from the 5′ end of exon 6 (SEQ ID NO: 4) of the human UMOD gene pre-mRNA;
(viii) within the 123^{rd} nucleotide to the 149^{th} nucleotide measured from the 5′ end of exon 6 (SEQ ID NO: 4) of the human UMOD gene pre-mRNA;
(ix) within the 24^{th} nucleotide of exon 6 measured from 3′ end of exon 6 (SEQ ID NO: 4) to the 6^{th} nucleotide of intron 6 measured from 3′ end of exon 6 (SEQ ID NO: 4) of the human UMOD gene pre-mRNA; or
(x) within the 15^{th} nucleotide of exon 6 measured from 3′ end of exon 6 (SEQ ID NO: 4) to the 6^{th} nucleotide of intron 6 measured from 3′ end of exon 6 (SEQ ID NO: 4) of the human UMOD gene pre-mRNA.

211. The antisense oligomer, or a pharmaceutically acceptable salt thereof, of claim 201, wherein the target region is selected from H8A(−2+23), H8A(+51+75), H8A(+60+79), H8A(+61+80), H8A(+68+87), H8A(+69+88), H8A(+70+89), H8A(+76+95), H8A(+76+100), H8A(+77+96), H8A(+78+97), H8A(+79+98), H8A(+81+100), H8A(+82+101), H8A(+83+102), H8A(+86+105), H8A(+94+113), H8A(+95+114), H8A(+96+115), H8A(+101+125), H8A(+102+121), H8A(+103+122), H8A(+104+123), H8A(+105+124), H8A(+120+139), H8A(+121+140), H8A(+122+141), H8A(+126+150), H8A(+128+147), H8A(+129+148), and H8D(+12-13).

212. The antisense oligomer, or a pharmaceutically acceptable salt thereof, of claim 211, wherein the targeting sequence comprises a sequence selected from SEQ ID NOs: 82, 84-86, 89-96, 98-115, and 120.

213. The antisense oligomer, or a pharmaceutically acceptable salt thereof, of claim 201, wherein the target region is:
(i) within the 51^{st} and the 80^{th} nucleotide measured from the 5′ end of exon 8 (SEQ ID NO: 5) of the human UMOD gene pre-mRNA;
(ii) within the 68^{th} nucleotide to the 100^{th} nucleotide measured from the 5′ end of exon 8 (SEQ ID NO:5) of the human UMOD gene pre-mRNA;
(iii) within the 76^{th} and the 100^{th} nucleotide measured from the 5′ end of exon 8 (SEQ ID NO: 5) of the human UMOD gene pre-mRNA;
(iv) within the 81^{st} and the 105^{th} nucleotide measured from the 5′ end of exon 8 (SEQ ID NO: 5) of the human UMOD gene pre-mRNA;
(v) within the 94^{th} and the 124^{th} nucleotide measured from the 5′ end of exon 8 (SEQ ID NO: 5) of the human UMOD gene pre-mRNA;
(vi) within the 120^{th} and the 148^{th} nucleotide measured from the 5′ end of exon 8 (SEQ ID NO: 5) of the human UMOD gene pre-mRNA; or
(vii) within the 126^{th} and the 148^{th} nucleotide measured from the 5′ end of exon 8 (SEQ ID NO: 5) of the human UMOD gene pre-mRNA.

214. The antisense oligomer, or a pharmaceutically acceptable salt thereof, of claim 201, wherein the target region is selected from H9A(−5+20), H9A(+1+25), H9A(+51+75), and H9D(+7-18).

215. The antisense oligomer, or a pharmaceutically acceptable salt thereof, of claim 214, wherein the targeting sequence comprises a sequence selected from SEQ ID NOs: 121-124.

216. The antisense oligomer, or a pharmaceutically acceptable salt thereof claim 201, wherein the target region is within the 5^{th} nucleotide of intron 8 measured from the 5′ end of exon 9 (SEQ ID NO: 6) and the 25^{th} nucleotide of exon 9 measured from the 5′ end of exon 9 (SEQ ID NO: 6) of the human UMOD gene pre-mRNA.

217. The antisense oligomer, or a pharmaceutically acceptable salt thereof, of claim 200, wherein the antisense oligomer, or the pharmaceutically acceptable salt thereof, is selected from a peptide nucleic acid, a locked nucleic acid, a phosphorodiamidate morpholino oligomer (PMO), a 2′-OMe phosphorothioate oligomer, or a combination thereof.

218. The antisense oligomer, or a pharmaceutically acceptable salt thereof, of claim 200, wherein the antisense oligomer, or the pharmaceutically acceptable salt thereof, further comprises a delivery agent selected from a cell-penetrating peptide, an antibody, a fragment of an antibody, an antigen binding agent, at least one ligand, and a combination thereof.

219. The antisense oligomer, or a pharmaceutically acceptable salt thereof, of claim 218, wherein the antisense oligomer, or the pharmaceutically acceptable salt thereof, is covalently linked to a cell-penetrating peptide via a linker selected from a direct bond, a glycine amino acid, a proline amino acid, a glutamic acid amino acid, or an isoglutamine amino acid; and wherein the cell-penetrating peptide is selected from rTAT (SEQ ID NO: 179), TAT (SEQ ID NO: 180), R₉F₂ (SEQ ID NO:181), R₅F₂R₄ (SEQ ID NO: 182), R₄ (SEQ ID NO: 183), R₅ (SEQ ID NO: 184), R₆ (SEQ ID NO: 185), R₇ (SEQ ID NO: 136), R₈ (SEQ ID NO: 137), R₉ (SEQ ID NO: 138), (RXR)₄ (SEQ ID NO: 139), (RXR) s (SEQ ID NO: 140), (RXRRBR)₂ (SEQ ID NO: 141), (RAR)₄F₂ (SEQ ID NO: 142), (RGR)₄F₂ (SEQ ID NO: 143), and RBRBYLIQFRBRRBR (SEQ ID NO: 144), wherein B represents beta-alanine (also represented as β-Ala or β), F represents phenylalanine, G represents glycine, L represents leucine, Q represents glutamine, R represents arginine, X represents 6-aminohexanoic acid (also represented as Ahx or «), Y represents tyrosine, I represents isoleucine, A represents alanine, and T represents threonine.

220. The antisense oligomer of claim 200 having a structure of Formula (I): 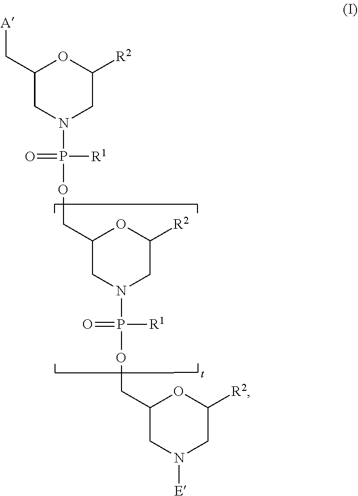
or a pharmaceutically acceptable salt thereof,
wherein:
A′ is selected from —OH. 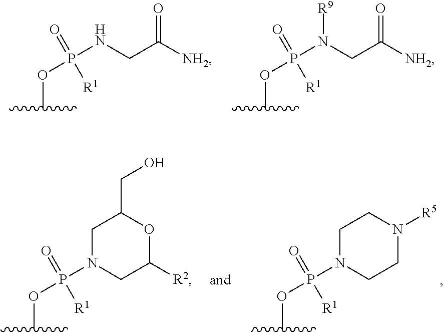
wherein
R⁵ is —C(O)(O-alkyl)ₓ-OH, wherein x is 3-10 and each alkyl group is, independently at each occurrence, C₂₋₆-alkyl,
or R⁵ is selected from H, —C(O) C₁₋₆-alkyl, trityl, monomethoxytrityl, —(C₁₋₆-alkyl)-R⁶, —(C₁₋₆-heteroalkyl)-R⁶, —C₆₋₁₀-aryl-R⁶, 5-10 membered heteroaryl-R⁶, —C(O)O—(C₁₋₆-alkyl)-R⁶, —C(O)O-aryl-R⁶, —C(O)O-(5-10 membered heteroaryl)-R⁶, and 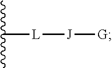
R⁶ is selected from —OH, —SH, and —NH₂, or R⁶ is O, S, or NH, each of which is covalently linked to a solid support;
R⁹ is C₁₋₆ alkyl;
each R¹ is independently selected from —OH and —N(R³)(R⁴), wherein each R³ and R⁴ is, independently at each occurrence, —H or —C₁₋₆-alkyl;
each R² is independently selected from a naturally or non-naturally occurring nucleobase, which, when taken together, forms the targeting sequence;
t is 11-28;
E′ is selected from —H, —C₁₋₆-alkyl, —C(O) C₁₋₆-alkyl, benzoyl, stearoyl, trityl, monomethoxytrityl, dimethoxytrityl, trimethoxytrityl, 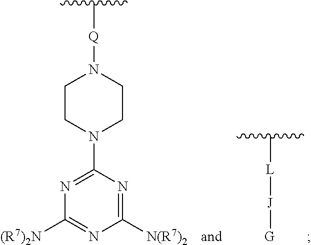
wherein:
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH) NH₂;
L is a linking amino acid, wherein L is covalently-linked by an amide bond to the C-terminus of J;
J is a cell-penetrating peptide; and
G is selected from —H, —C(O) C₁₋₆-alkyl, benzoyl, and stearoyl, wherein G is covalently-linked to J.

221. A pharmaceutical composition comprising the antisense oligomer, or a pharmaceutically acceptable salt thereof, of claim 220, and a pharmaceutically acceptable carrier.

222. A method of treating a disease associated with aberrant expression of uromodulin protein, comprising administering to a subject in need thereof a therapeutically effective amount of the antisense oligomer, or a pharmaceutically acceptable salt thereof, of claim 200.

223. The method of claim 222, wherein the disease is a chronic kidney disease (CKD).

224. The method of claim 223, wherein the autosomal dominant renal disorder is an autosomal dominant tubulointerstitial kidney disease (ADTKD).


SEQUENCE LISTING (564 sequences)

SEQ ID NO: 1 - DNA, 7866 bp, Homo sapiens
  source 1..7866: mol_type = genomic DNA; note = Human UMOD gene
agatcatgtggcatagagcccctgacaggccacgagccagagaatgggtgtgccacctccaggtcccccaggcgtaactggagcctcaggaggtgtgtgctcagagaggtaggtgggacaggtaatacccctcttttgggggatcttctcccttggctcattttttgccgctttctgacacacaccaacctctcattctaagtttttgtgttctgtttgatcaatttttcaaaactctccaccctttagtttcactctttccaaaactacctaaaaccacttatccttctaagaccctgtcatggctgagcatggaatgttctgtaacccaaggaatgcaggtaaatctgagtgagatttgagttttagtctggatttaacactcgctagctgtgtaatcttgggggagttactccacctctctgaacttgtcttcttccctgtgacgtgggttgttgtgtttcattgtttgagacaatggaggcaaaagcactgagcacagtgtctgacccaagagaagtgctcagtgggtgattgttctcagcattattaagatcaagtctcctacccatgtttgaaagttaagagagaaaagtgtttcaggctggttgtggtggctcatgtctgtaatcccagcactttgggaggccgaggcaggcagattacctgaggtcaggagttcgagaccagcctggccaatgtgtacaaaaaaaacacaaattagccgggcatggtggtatgcacctgtaatcccagctactcgggaggctgaggattcaaggattcaagagaatcacttgaacttgggaggtggaggttgcagtgagccaagactgagccactgcactccaacctgggtgacagagcaagactccattaaaaaaaaaaaaaagaagaagaaaaagaaagaaagaaagaagagtgtttcaagagcgaagtgctgagagattaaggttcgttcaaaaacaaatatttattgtgcactgtcctagaggtaagataaagagaaaaatattgattgaatttggtaacatggatgtctttggtgaacttgatgagcaacagagcaggaaattatggccactgacaacttgttatactcacagatcccaagagaaggggacacaccacactgggcagaggggctgaagggagaggaagagctgctgggaagtaccagggtcggtcaggaagcaaagagagcaagggaaaaggcaggaaagagcctttattgtagtttctgcaggaaggaatgggagaggcaaggtaagcagatttaggattggcttagtttgaataatttcagagacctctgggccataggagttgtctctagttgtctggtacctggtcccttggggtgatctaaagcagcaagtagtgaccctaagtgtaaaagccccaaaaaggaggccaagggttgggggtatgtggtctggattgattgatttgcagtcggaaaggcacattcacagataagtcgtttttgatctctagtaattattgagccctgggaggggcagtccctccatgatcagtaaggccccaaaatgtacaagcaccaaatacagaaactagaaagcatggtgattacacttatgaatagattccaacccaaactccccattttcctgcccaagtctttgtcaagaaaactccagaagccacctggggtcactgtctttaatcctttgaagacacccataataaggtttagacaaggcctcatgaaaaaggtataggataaaaacgactagttcagaaaaaaaaaagtaaataaatgaagagataacttcccttttcatcaaagaattgcaaaaatgaacaataatgagatcgtttccacttatccttttggcaaagatgaaagcaagttaccaatgctcagaactggtgagtagtgttggtgggattgtaaaccaatttataataattctctggggtgctgtttgggaagaggagtcaatattcctacagctgtgctacctctttgacccagaatttccactccttggacataatcctgaggagatgacaacaccagtgccagtggttaccctaccactgagtgggacaggatggagaaatgtcctgaaaagagcaattcacaatagcaaagacttggaaccaactcaaatgtccatcaatgatagactggattaagaaaatgtggcacatatacaccgtggaatactatgcagccataaaaaatatgagttcatgtcctttgtagggacatggatgaagatggaaaccatcattctcagcaaactatcgcaaggacaaaaaaccaaacaccgcatgttctcactcacaggtgggaattgaacaatgagagcacttggacacaggaaggggaacatcacacccaggggcctattgtgggatgcggggagcggggagggatagcattaggagttatacctaatgtaaatgacaagttaatgggtgcagcacatcaacatggcacatgtatacatatgtaacaaacctgcacgttgtgcacttgtaccctaaaacttaaagtataaaaaaaaaaaagatcaatgcagtgatcatggtgatattttcctgctcagcccaagttcacacatattttatttttctcaacatgatgacagccactctcacactgacttttggaatgtcatgtatgttgaactgggtctgaagacatggttttaactcaggctctgtcattttctacctcagtgattgcacaacagcaaagcagaattttcactacttccatgaatataatcattactatatgactttacttgcatcatctcctttggttactattactactgtgggagatgggtattctcattttatagacaaggaaattgacctctggacctcaggaaggttaagaaatgagcccactgccacacaataaacaccagataaaggaggcagactgactccaaagtcagtctatttaagtgcaaatttatttcgcctccaaagggacctcccagtcatcagacctgattctttgttgtacagagtgggtcaggtccagtgatgtctgaactaccttctggttctgactttcagccattctcagctcctctcttgcttgtgtctggattctaaggctgatctcatgagaatgggtgtttcagaagggtgccctctccaagacaggtgcacctcccatctggggcagtgaatatcccttttgtccttatgcagcctggcttcagatactggcttctgcctggctccttgatcccaccctgcccttgtcagtgaccaagaagaagcccagcaccttggcactgctttcccagttaatttctaactatggaatctcttgctgttagaaggtgcgaaacagtgaccttgtatttccgggcacaggtgtgaccccccaatgtcaatcatttggggtctctagctattaggaaaaagaacaacaacaacctcacagcttggacaaggcaaacattatgccaggaggaaaaaatattccacccccaagaaaacaatatcaaaaaacagaactagagactaattggaggagagattgccagcctggggcaaatgtgtatatataagtatgaggcacatcatgaccagactaactctacctttctggcttcaggtaaggctatctgtagctgtcttctcctagcccagcttctccccatcctatttgagggaggtaggagaggaaattaagaccttggacactggggtcagacctggatttgagtcattattctgccaattattggcttcatgatctttagtaagttagttttcctctctttgatcctctctgtcctagtaacaatgactactattttttgacttattccatgaatattagttatgcacctattatgtgccaggtaccagggttacaacaatgaacaacttgctatgtgctgggccctgctcgacatatgtcatcttagttaatcccagtgacaatatgcagtgttttaggtcgcatcttattagcgggtcataaaatcattccttttgtgggttatatccagggacttcttttaattaaatataatagaatatgtcagtaagcaatacaaatttgaggcaagcagttagcctctttgtccccctgttttctcatctgcaaactaggataatacttatctcatagggttgtggtaagaattaaatcacttaatgtagtaaggatatcaattgtcttataaaattttatgtgtgtgtgcacatacatgcagaagcgactgcactagatcatgacataaaatgtttttctttctttctgtctttttttttttgggggggggggatggactctctgtcacccaggcaggagtgcagtggcacgatctcagctcactgcaacctctgcctcccaggttcaagcaatcctcctgtctcagcctcccgagtagctgggattacaggcgcccaccaccatgcccggctaatttttgtatttttattagagatggggtttcaccatgctggccaggctggtctcaaactcctgaccttgtgatccaccccctcagtctcccaaagtgttgggattacaggcatgagccactgcacctggcaacataaaatgtttttcttttagtacatcatggtcacgaagatctggaaaacgttgctctaggacccaatgcctttgagcacatgaatgtctttgaactgagcctggcccatcatagatattcagtttgtgaaagcaacaatctccatttcacagacaaaaacggagacttagtaaactgaaatacccacatcacagtgttagtgatcagtaaagcagggatttgaacctcaggtgcaggctgcagctcctacatacttaaccaacatgccaccctattttgctgctataaacactggggtaatactggcatccaccctggactatttttccaattggaagaaatagtaagtgaccactccagaaacatttaaatgcattgtgaaggttaattacctctcaagttgctcgtctcaagccacttctttacgatatgtgagttgcatgaaggagcagactgccatgttcattgttctccatggtgactagctccctgtttggcacaatgtagctactcaatagatgtttgttgaatgaataagtgatccaggcaaattgaaagtttccaggcagaggatcagttaaaatccaaagctgccttcagtaaagtggtgagtccagaacactattccatctggctacttcctgctccaaatgactgagttcttcaaaatgtgcaatgtgctgagaattggggagccaagactgggatgttggtgaggtaaggagggggagtacaaggggtaaagtcccagcaaaacaagggctgcagtgttatgcaattttttagtccatataagtgacacctcctggagttgtatactatacaatcaaagcactccttccagctgtggggaggagagttagatcatgcatttgtcccatccatctctgttcacaggacaccagacatcagagacagagagaaaaattcaaagggccaacccgtctttcctttgggcaggtgctatctagacctgaagtagcgggaagagcagaaaggatggggcagccatctctgacttggatgctgatggtggtggtggcctcttggttcatcacaactgcagccactgacacctcagaagcaagtaagtgaaaagtgtgtgtgcgttgtatatgtgtgtatgcacgtgtatgtgtgaatgtgtgggggaagcaatgtagcacctgtcagaggtgatctcaatcctcctatcgcacttgagacttgcattgtcttcattctaagtccatttcttagactcaatatgcacaggactgacttagaaattttgctaaagtgcatattctggttcagcagatctgcagtaggacctgagatgctgaatttttaacaagcttccaggcgatgctcatactggggtccctggagtacacattgaaaagcaaggggctagaacatctctaaggcctgcaggccctttattggaagtcagaaacatactctatcacataggagatttgaacccatgcaggaggatccaaacaccttccctttcaactttaagaggtcattccattgggttgagatttgctgtcaccccactttcattttctccctggagtacgttggggcacgatgaatactattgcggtgtcctggttaaaagcacatattttgtggtcctgccatctgcgtttttatcctggttctacttcttaccaaaggagtaaggggcttaatccctctgaacctcagtctcctcatctttaaaataaggatacataaaaaactgacctcacgaggcccttgggaagtattcaacaagatagtgagtgaaaagtgcacatcctattgcctggcatatagtaattgcttaataaacaacagcttctttttttttttaatggttatttttattacggaggaacaaagtacaactgcccagccaggtggagttggaggactccgcagagaggaggcgacactgagcagggtcctgatgaagatttcaccagccagggaagcagaaaacataaaatgtgcaaagaaagggaggggcaacaggttcaccgtaaatcctaccaaagtataggaattctgcgcattacttttctgaatgtggctattttaaaagaagacagcttgaaagcaatgcttaacacaaaaaatgaatggtggagctgggcgcgattgcacgtgcctgtggtcccaactttttgggaagctgaggcagggtgcggtggtggcttgaactcagggagtagaacgcttgaacccaggaattggaggctatggtgagctatgatcgcactactgcactccagcctgggcgacagagcaagacccctctccaaaaaaataaataaagttaaaaaatacaatgaaataaacacagaatgaacggtggaggcttgacatcatcagaggagttttgtttctttgcttctttccttgttttggagggagccctctagggaataagtcttaaaaataatgagttccctggagaatgagggaaggatctctgggtggccatgggcccagctgcccaaaccctgaagctgggcttttctgtccacaggatggtgctctgaatgtcacagcaatgccacctgcacggaggatgaggccgttacgacgtgcacctgtcaggagggcttcaccggcgatggcctgacctgcgtggacctggatgagtgcgccattcctggagctcacaactgctccgccaacagcagctgcgtaaacacgccaggctccttctcctgcgtctgccccgaaggcttccgcctgtcgcccggtctcggctgcacagacgtggatgagtgcgctgagcctgggcttagccactgccacgccctggccacatgtgtcaatgtggtgggcagctacttgtgcgtatgccccgcgggctaccggggggatggatggcactgtgagtgctccccgggctcctgcgggccggggttggactgcgtgcccgagggcgacgcgctcgtgtgcgcggatccgtgccaggcgcaccgcaccctggacgagtactggcgcagcaccgagtacggggagggctacgcctgcgacacggacctgcgcggctggtaccgcttcgtgggccagggcggtgcgcgcatggccgagacctgcgtgccagtcctgcgctgcaacacggccgcccccatgtggctcaatggcacgcatccgtccagcgacgagggcatcgtgagccgcaaggcctgcgcgcactggagcggccactgctgcctgtgggatgcgtccgtccaggtgaaggcctgtgccggcggctactacgtctacaacctgacagcgccccccgagtgtcacctggcgtactgcacag

SEQ ID NO: 2 - DNA, 218 bp, synthetic construct
  source 1..218: mol_type = other DNA; note = Exon 2 with junctions
atccatctctgttcacaggacaccagacatcagagacagagagaaaaattcaaagggccaacccgtctttcctttgggcaggtgctatctagacctgaagtagcgggaagagcagaaaggatggggcagccatctctgacttggatgctgatggtggtggtggcctcttggttcatcacaactgcagccactgacacctcagaagcaagtaagtgaaa

SEQ ID NO: 3 - DNA, 234 bp, synthetic construct
  source 1..234: mol_type = other DNA; note = Exon 5 with junctions
cctaacctgtttcagatatctccctcctggagcacaggctggaatgtggggccaatgacatgaaggtgtcgctgggcaagtgccagctgaagagtctgggcttcgacaaggtcttcatgtacctgagtgacagccggtgctcgggcttcaatgacagagacaaccgggactgggtgtctgtagtgaccccagcccgggatggcccctgtgggacagtgttgacggtacgtcctg

SEQ ID NO: 4 - DNA, 155 bp, synthetic construct
  source 1..155: mol_type = other DNA; note = Exon 6 with junctions
aggaatgaaacccatgccacttacagcaacaccctctacctggcagatgagatcatcatccgtgacctcaacatcaaaatcaactttgcatgctcctaccccctggacatgaaagtcagcctgaagaccgccctacagccaatggtcaggtgtgg

SEQ ID NO: 5 - DNA, 177 bp, synthetic construct
  source 1..177: mol_type = other DNA; note = Exon 8 with junctions
agatgcccacacactagagactcaactatccaagtggtggagaatggggagtcctcccagggccgattttccgtccagatgttccggtttgctggaaactatgacctagtctacctgcactgtgaagtctatctctgtgacaccatgaatgaaaagtgcaagcctgtgagttgactc

SEQ ID NO: 6 - DNA, 105 bp, synthetic construct
  source 1..105: mol_type = other DNA; note = Exon 9 with junctions
tgtagacctgctctgggaccagattccgaagtgggagtgtcatagatcaatcccgtgtcctgaacttgggtcccatcacacggaaaggtaagagagccactcgct

SEQ ID NO: 7 - DNA, 25 bp, synthetic construct
  source 1..25: mol_type = other DNA
ctgtgaacagagatggatgggacaa

SEQ ID NO: 8 - DNA, 20 bp, synthetic construct
  source 1..20: mol_type = other DNA
tcctgtgaacagagatggat

SEQ ID NO: 9 - DNA, 20 bp, synthetic construct
  source 1..20: mol_type = other DNA
gtcctgtgaacagagatgga

SEQ ID NO: 10 - DNA, 20 bp, synthetic construct
  source 1..20: mol_type = other DNA
tgtcctgtgaacagagatgg

SEQ ID NO: 11 - DNA, 25 bp, synthetic construct
  source 1..25: mol_type = other DNA
gtctggtgtcctgtgaacagagatg

SEQ ID NO: 12 - DNA, 25 bp, synthetic construct
  source 1..25: mol_type = other DNA
ctctctgtctctgatgtctggtgtc

SEQ ID NO: 13 - DNA, 25 bp, synthetic construct
  source 1..25: mol_type = other DNA
agacgggttggccctttgaattttt

SEQ ID NO: 14 - DNA, 25 bp, synthetic construct
  source 1..25: mol_type = other DNA
tctagatagcacctgcccaaaggaa

SEQ ID NO: 15 - DNA, 20 bp, synthetic construct
  source 1..20: mol_type = other DNA
atcctttctgctcttcccgc

SEQ ID NO: 16 - DNA, 20 bp, synthetic construct
  source 1..20: mol_type = other DNA
catcctttctgctcttcccg

SEQ ID NO: 17 - DNA, 25 bp, synthetic construct
  source 1..25: mol_type = other DNA
agagatggctgccccatcctttctg

SEQ ID NO: 18 - DNA, 20 bp, synthetic construct
  source 1..20: mol_type = other DNA
atggctgccccatcctttct

SEQ ID NO: 19 - DNA, 20 bp, synthetic construct
  source 1..20: mol_type = other DNA
agatggctgccccatccttt

SEQ ID NO: 20 - DNA, 25 bp, synthetic construct
  source 1..25: mol_type = other DNA
caagtcagagatggctgccccatcc

SEQ ID NO: 21 - DNA, 20 bp, synthetic construct
  source 1..20: mol_type = other DNA
tccaagtcagagatggctgc

SEQ ID NO: 22 - DNA, 20 bp, synthetic construct
  source 1..20: mol_type = other DNA
atccaagtcagagatggctg

SEQ ID NO: 23 - DNA, 20 bp, synthetic construct
  source 1..20: mol_type = other DNA
catccaagtcagagatggct

SEQ ID NO: 24 - DNA, 20 bp, synthetic construct
  source 1..20: mol_type = other DNA
cagcatccaagtcagagatg

SEQ ID NO: 25 - DNA, 20 bp, synthetic construct
  source 1..20: mol_type = other DNA
tcagcatccaagtcagagat

SEQ ID NO: 26 - DNA, 20 bp, synthetic construct
  source 1..20: mol_type = other DNA
accatcagcatccaagtcag

SEQ ID NO: 27 - DNA, 25 bp, synthetic construct
  source 1..25: mol_type = other DNA
agaggccaccaccaccatcagcatc

SEQ ID NO: 28 - DNA, 25 bp, synthetic construct
  source 1..25: mol_type = other DNA
cagtggctgcagttgtgatgaacca

SEQ ID NO: 29 - DNA, 25 bp, synthetic construct
  source 1..25: mol_type = other DNA
tttcacttacttgcttctgaggtgt

SEQ ID NO: 30 - DNA, 20 bp, synthetic construct
  source 1..20: mol_type = other DNA
gatatctgaaacaggttagg

SEQ ID NO: 31 - DNA, 20 bp, synthetic construct
  source 1..20: mol_type = other DNA
agatatctgaaacaggttag

SEQ ID NO: 32 - DNA, 20 bp, synthetic construct
  source 1..20: mol_type = other DNA
gggagatatctgaaacaggt

SEQ ID NO: 33 - DNA, 20 bp, synthetic construct
  source 1..20: mol_type = other DNA
agggagatatctgaaacagg

SEQ ID NO: 34 - DNA, 18 bp, synthetic construct
  source 1..18: mol_type = other DNA
cacttgcccagcgacacc

SEQ ID NO: 35 - DNA, 25 bp, synthetic construct
  source 1..25: mol_type = other DNA
tcagctggcacttgcccagcgacac

SEQ ID NO: 36 - DNA, 18 bp, synthetic construct
  source 1..18: mol_type = other DNA
ggcacttgcccagcgaca

SEQ ID NO: 37 - DNA, 25 bp, synthetic construct
  source 1..25: mol_type = other DNA
aagaccttgtcgaagcccagactct

SEQ ID NO: 38 - DNA, 18 bp, synthetic construct
  source 1..18: mol_type = other DNA
cttgtcgaagcccagact

SEQ ID NO: 39 - DNA, 18 bp, synthetic construct
  source 1..18: mol_type = other DNA
accttgtcgaagcccaga

SEQ ID NO: 40 - DNA, 18 bp, synthetic construct
  source 1..18: mol_type = other DNA
agaccttgtcgaagccca

SEQ ID NO: 41 - DNA, 18 bp, synthetic construct
  source 1..18: mol_type = other DNA
tgaagaccttgtcgaagc

SEQ ID NO: 42 - DNA, 18 bp, synthetic construct
  source 1..18: mol_type = other DNA
atgaagaccttgtcgaag

SEQ ID NO: 43 - DNA, 18 bp, synthetic construct
  source 1..18: mol_type = other DNA
ggtacatgaagaccttgt

SEQ ID NO: 44 - DNA, 25 bp, synthetic construct
  source 1..25: mol_type = other DNA
ggttgtctctgtcattgaagcccga

SEQ ID NO: 45 - DNA, 20 bp, synthetic construct
  source 1..20: mol_type = other DNA
tcactacagacacccagtcc

SEQ ID NO: 46 - DNA, 20 bp, synthetic construct
  source 1..20: mol_type = other DNA
gtcactacagacacccagtc

SEQ ID NO: 47 - DNA, 20 bp, synthetic construct
  source 1..20: mol_type = other DNA
ggtcactacagacacccagt

SEQ ID NO: 48 - DNA, 20 bp, synthetic construct
  source 1..20: mol_type = other DNA
accgtcaacactgtcccaca

SEQ ID NO: 49 - DNA, 20 bp, synthetic construct
  source 1..20: mol_type = other DNA
taccgtcaacactgtcccac

SEQ ID NO: 50 - DNA, 20 bp, synthetic construct
  source 1..20: mol_type = other DNA
gtaccgtcaacactgtccca

SEQ ID NO: 51 - DNA, 20 bp, synthetic construct
  source 1..20: mol_type = other DNA
acgtaccgtcaacactgtcc

SEQ ID NO: 52 - DNA, 20 bp, synthetic construct
  source 1..20: mol_type = other DNA
gacgtaccgtcaacactgtc

SEQ ID NO: 53 - DNA, 20 bp, synthetic construct
  source 1..20: mol_type = other DNA
ggacgtaccgtcaacactgt

SEQ ID NO: 54 - DNA, 20 bp, synthetic construct
  source 1..20: mol_type = other DNA
aggacgtaccgtcaacactg

SEQ ID NO: 55 - DNA, 20 bp, synthetic construct
  source 1..20: mol_type = other DNA
caggacgtaccgtcaacact

SEQ ID NO: 56 - DNA, 25 bp, synthetic construct
  source 1..25: mol_type = other DNA
tgtaagtggcatgggtttcattcct

SEQ ID NO: 57 - DNA, 25 bp, synthetic construct
  source 1..25: mol_type = other DNA
tcatctgccaggtagagggtgttgc

SEQ ID NO: 58 - DNA, 20 bp, synthetic construct
  source 1..20: mol_type = other DNA
ggtcacggatgatgatctca

SEQ ID NO: 59 - DNA, 20 bp, synthetic construct
  source 1..20: mol_type = other DNA
aggtcacggatgatgatctc

SEQ ID NO: 60 - DNA, 20 bp, synthetic construct
  source 1..20: mol_type = other DNA
gaggtcacggatgatgatct

SEQ ID NO: 61 - DNA, 20 bp, synthetic construct
  source 1..20: mol_type = other DNA
ttgatgttgaggtcacggat

SEQ ID NO: 62 - DNA, 20 bp, synthetic construct
  source 1..20: mol_type = other DNA
tttgatgttgaggtcacgga

SEQ ID NO: 63 - DNA, 20 bp, synthetic construct
  source 1..20: mol_type = other DNA
ttttgatgttgaggtcacgg

SEQ ID NO: 64 - DNA, 25 bp, synthetic construct
  source 1..25: mol_type = other DNA
ggtaggagcatgcaaagttgatttt

SEQ ID NO: 65 - DNA, 20 bp, synthetic construct
  source 1..20: mol_type = other DNA
taggagcatgcaaagttgat

SEQ ID NO: 66 - DNA, 25 bp, synthetic construct
  source 1..25: mol_type = other DNA
ttcaggctgactttcatgtccaggg

SEQ ID NO: 67 - DNA, 20 bp, synthetic construct
  source 1..20: mol_type = other DNA
gctgactttcatgtccaggg

SEQ ID NO: 68 - DNA, 20 bp, synthetic construct
  source 1..20: mol_type = other DNA
ggtcttcaggctgactttca

SEQ ID NO: 69 - DNA, 20 bp, synthetic construct
  source 1..20: mol_type = other DNA
cggtcttcaggctgactttc

SEQ ID NO: 70 - DNA, 20 bp, synthetic construct
  source 1..20: mol_type = other DNA
gcggtcttcaggctgacttt

SEQ ID NO: 71 - DNA, 20 bp, synthetic construct
  source 1..20: mol_type = other DNA
ggcggtcttcaggctgactt

SEQ ID NO: 72 - DNA, 20 bp, synthetic construct
  source 1..20: mol_type = other DNA
ctgtagggcggtcttcaggc

SEQ ID NO: 73 - DNA, 20 bp, synthetic construct
  source 1..20: mol_type = other DNA
gctgtagggcggtcttcagg

SEQ ID NO: 74 - DNA, 20 bp, synthetic construct
  source 1..20: mol_type = other DNA
ggctgtagggcggtcttcag

SEQ ID NO: 75 - DNA, 20 bp, synthetic construct
  source 1..20: mol_type = other DNA
tggctgtagggcggtcttca

SEQ ID NO: 76 - DNA, 20 bp, synthetic construct
  source 1..20: mol_type = other DNA
ttggctgtagggcggtcttc

SEQ ID NO: 77 - DNA, 20 bp, synthetic construct
  source 1..20: mol_type = other DNA
attggctgtagggcggtctt

SEQ ID NO: 78 - DNA, 20 bp, synthetic construct
  source 1..20: mol_type = other DNA
ctgaccattggctgtagggc

SEQ ID NO: 79 - DNA, 25 bp, synthetic construct
  source 1..25: mol_type = other DNA
cctgaccattggctgtagggcggtc

SEQ ID NO: 80 - DNA, 20 bp, synthetic construct
  source 1..20: mol_type = other DNA
cacacctgaccattggctgt

SEQ ID NO: 81 - DNA, 20 bp, synthetic construct
  source 1..20: mol_type = other DNA
ccacacctgaccattggctg

SEQ ID NO: 82 - DNA, 25 bp, synthetic construct
  source 1..25: mol_type = other DNA
ttgagtctctagtgtgtgggcatct

SEQ ID NO: 83 - DNA, 25 bp, synthetic construct
  source 1..25: mol_type = other DNA
actccccattctccaccacttggat

SEQ ID NO: 84 - DNA, 25 bp, synthetic construct
  source 1..25: mol_type = other DNA
tggacggaaaatcggccctgggagg

SEQ ID NO: 85 - DNA, 20 bp, synthetic construct
  source 1..20: mol_type = other DNA
catctggacggaaaatcggc

SEQ ID NO: 86 - DNA, 20 bp, synthetic construct
  source 1..20: mol_type = other DNA
acatctggacggaaaatcgg

SEQ ID NO: 87 - DNA, 20 bp, synthetic construct
  source 1..20: mol_type = other DNA
aacatctggacggaaaatcg

SEQ ID NO: 88 - DNA, 20 bp, synthetic construct
  source 1..20: mol_type = other DNA
gaacatctggacggaaaatc

SEQ ID NO: 89 - DNA, 20 bp, synthetic construct
  source 1..20: mol_type = other DNA
aaccggaacatctggacgga

SEQ ID NO: 90 - DNA, 20 bp, synthetic construct
  source 1..20: mol_type = other DNA
aaaccggaacatctggacgg

SEQ ID NO: 91 - DNA, 20 bp, synthetic construct
  source 1..20: mol_type = other DNA
caaaccggaacatctggacg

SEQ ID NO: 92 - DNA, 20 bp, synthetic construct
  source 1..20: mol_type = other DNA
ttccagcaaaccggaacatc

SEQ ID NO: 93 - DNA, 25 bp, synthetic construct
  source 1..25: mol_type = other DNA
atagtttccagcaaaccggaacatc

SEQ ID NO: 94 - DNA, 20 bp, synthetic construct
  source 1..20: mol_type = other DNA
tttccagcaaaccggaacat

SEQ ID NO: 95 - DNA, 20 bp, synthetic construct
  source 1..20: mol_type = other DNA
gtttccagcaaaccggaaca

SEQ ID NO: 96 - DNA, 20 bp, synthetic construct
  source 1..20: mol_type = other DNA
agtttccagcaaaccggaac

SEQ ID NO: 97 - DNA, 20 bp, synthetic construct
  source 1..20: mol_type = other DNA
tagtttccagcaaaccggaa

SEQ ID NO: 98 - DNA, 20 bp, synthetic construct
  source 1..20: mol_type = other DNA
atagtttccagcaaaccgga

SEQ ID NO: 99 - DNA, 20 bp, synthetic construct
  source 1..20: mol_type = other DNA
catagtttccagcaaaccgg

SEQ ID NO: 100 - DNA, 20 bp, synthetic construct
  source 1..20: mol_type = other DNA
tcatagtttccagcaaaccg

SEQ ID NO: 101 - DNA, 20 bp, synthetic construct
  source 1..20: mol_type = other DNA
aggtcatagtttccagcaaa

SEQ ID NO: 102 - DNA, 20 bp, synthetic construct
  source 1..20: mol_type = other DNA
ggtagactaggtcatagttt

SEQ ID NO: 103 - DNA, 20 bp, synthetic construct
  source 1..20: mol_type = other DNA
aggtagactaggtcatagtt

SEQ ID NO: 104 - DNA, 20 bp, synthetic construct
  source 1..20: mol_type = other DNA
caggtagactaggtcatagt

SEQ ID NO: 105 - DNA, 25 bp, synthetic construct
  source 1..25: mol_type = other DNA
cttcacagtgcaggtagactaggtc

SEQ ID NO: 106 - DNA, 20 bp, synthetic construct
  source 1..20: mol_type = other DNA
acagtgcaggtagactaggt

SEQ ID NO: 107 - DNA, 20 bp, synthetic construct
  source 1..20: mol_type = other DNA
cacagtgcaggtagactagg

SEQ ID NO: 108 - DNA, 20 bp, synthetic construct
  source 1..20: mol_type = other DNA
tcacagtgcaggtagactag

SEQ ID NO: 109 - DNA, 20 bp, synthetic construct
  source 1..20: mol_type = other DNA
ttcacagtgcaggtagacta

SEQ ID NO: 110 - DNA, 20 bp, synthetic construct
  source 1..20: mol_type = other DNA
gtcacagagatagacttcac

SEQ ID NO: 111 - DNA, 20 bp, synthetic construct
  source 1..20: mol_type = other DNA
tgtcacagagatagacttca

SEQ ID NO: 112 - DNA, 20 bp, synthetic construct
  source 1..20: mol_type = other DNA
gtgtcacagagatagacttc

SEQ ID NO: 113 - DNA, 25 bp, synthetic construct
  source 1..25: mol_type = other DNA
tcattcatggtgtcacagagataga

SEQ ID NO: 114 - DNA, 20 bp, synthetic construct
  source 1..20: mol_type = other DNA
ttcatggtgtcacagagata

SEQ ID NO: 115 - DNA, 20 bp, synthetic construct
  source 1..20: mol_type = other DNA
attcatggtgtcacagagat

SEQ ID NO: 116 - DNA, 20 bp, synthetic construct
  source 1..20: mol_type = other DNA
tttcattcatggtgtcacag

SEQ ID NO: 117 - DNA, 20 bp, synthetic construct
  source 1..20: mol_type = other DNA
ttttcattcatggtgtcaca

SEQ ID NO: 118 - DNA, 20 bp, synthetic construct
  source 1..20: mol_type = other DNA
tgcacttttcattcatggtg

SEQ ID NO: 119 - DNA, 20 bp, synthetic construct
  source 1..20: mol_type = other DNA
aggcttgcacttttcattca

SEQ ID NO: 120 - DNA, 25 bp, synthetic construct
  source 1..25: mol_type = other DNA
gagtcaactcacaggcttgcacttt

SEQ ID NO: 121 - DNA, 25 bp, synthetic construct
  source 1..25: mol_type = other DNA
aatctggtcccagagcaggtctaca

SEQ ID NO: 122 - DNA, 25 bp, synthetic construct
  source 1..25: mol_type = other DNA
ttcggaatctggtcccagagcaggt

SEQ ID NO: 123 - DNA, 25 bp, synthetic construct
  source 1..25: mol_type = other DNA
tgtgatgggacccaagttcaggaca

SEQ ID NO: 124 - DNA, 25 bp, synthetic construct
  source 1..25: mol_type = other DNA
agcgagtggctctcttacctttccg

SEQ ID NO: 125 - DNA, 20 bp, synthetic construct
  source 1..20: mol_type = other DNA
gagggagatatctgaaacag

SEQ ID NO: 126 - DNA, 25 bp, synthetic construct
  source 1..25: mol_type = other DNA
actctgccctctgtcctggaaacac

SEQ ID NO: 127 - DNA, 25 bp, synthetic construct
  source 1..25: mol_type = other DNA
ggtcaaagggatccccatcctttac

SEQ ID NO: 128 - DNA, 25 bp, synthetic construct
  source 1..25: mol_type = other DNA
ttaccatcattaccagcagcatcca

SEQ ID NO: 129 - DNA, 25 bp, synthetic construct
  source 1..25: mol_type = other DNA
cttgatgtcattggccccacactcc

SEQ ID NO: 130 - DNA, 25 bp, synthetic construct
  source 1..25: mol_type = other DNA
ctgaaaatggtcaggaacaacagga

SEQ ID NO: 131 - DNA, 25 bp, synthetic construct
  source 1..25: mol_type = other DNA
tgggagacatctgaaaatggtcagg

SEQ ID NO: 132 - DNA, 26 bp, synthetic construct
  source 1..26: mol_type = other DNA
tggttgtatcttctgtacgtggacaa

SEQ ID NO: 133 - DNA, 25 bp, synthetic construct
  source 1..25: mol_type = other DNA
gactcgccattctctgtcacctgaa

SEQ ID NO: 134 - DNA, 25 bp, synthetic construct
  source 1..25: mol_type = other DNA
ttcgaaatctagtaccagagcaggt

SEQ ID NO: 135 - DNA, 18 bp, synthetic construct
  source 1..18: mol_type = other DNA
gctattaccttaacccag

SEQ ID NO: 136 - protein, 7 aa, synthetic construct
  source 1..7: note = Cell-Penetrating Peptide
RRRRRRR

SEQ ID NO: 137 - protein, 8 aa, synthetic construct
  source 1..8: note = Cell-Penetrating Peptide
RRRRRRRR

SEQ ID NO: 138 - protein, 9 aa, synthetic construct
  source 1..9: note = Cell-Penetrating Peptide
RRRRRRRRR

SEQ ID NO: 139 - protein, 12 aa, synthetic construct
  source 1..12: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
RXRRXRRXRRXR

SEQ ID NO: 140 - protein, 15 aa, synthetic construct
  source 1..15: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 14: note = 6-aminohexanoic acid
RXRRXRRXRRXRRXR

SEQ ID NO: 141 - protein, 12 aa, synthetic construct
  source 1..12: note = Cell-Penetrating Peptide
  MOD_RES 11: note = bAla
  MOD_RES 5: note = bAla
  MOD_RES 2: note = 6-aminohexanoic acid
  MOD_RES 8: note = 6-aminohexanoic acid
RXRRBRRXRRBR

SEQ ID NO: 142 - protein, 15 aa, synthetic construct
  source 1..15: note = Cell-Penetrating Peptide
RARRARRARRARFFC

SEQ ID NO: 143 - protein, 15 aa, synthetic construct
  source 1..15: note = Cell-Penetrating Peptide
RGRRGRRGRRGRFFC

SEQ ID NO: 144 - protein, 15 aa, synthetic construct
  source 1..15: note = Cell-Penetrating Peptide
  MOD_RES 2: note = bAla
  MOD_RES 4: note = bAla
  MOD_RES 11: note = bAla
  MOD_RES 14: note = bAla
RBRBYLIQFRBRRBR

SEQ ID NO: 145 - DNA, 25 bp, synthetic construct
  source 1..25: mol_type = other DNA; note = PPMO1
  misc_feature 1..25: note = PPMO modification
actctgccctctgtcctggaaacac

SEQ ID NO: 146 - DNA, 25 bp, synthetic construct
  source 1..25: mol_type = other DNA; note = PPMO2
  misc_feature 1..25: note = PPMO modification
ggtcaaagggatccccatcctttac

SEQ ID NO: 147 - DNA, 25 bp, synthetic construct
  source 1..25: mol_type = other DNA; note = PPMO3
  misc_feature 1..25: note = PPMO modification
ttaccatcattaccagcagcatcca

SEQ ID NO: 148 - DNA, 25 bp, synthetic construct
  source 1..25: mol_type = other DNA; note = PPMO4
  misc_feature 1..25: note = PPMO modification
cttgatgtcattggccccacactcc

SEQ ID NO: 149 - DNA, 25 bp, synthetic construct
  source 1..25: mol_type = other DNA; note = PPMO5
  misc_feature 1..25: note = PPMO modification
ctgaaaatggtcaggaacaacagga

SEQ ID NO: 150 - DNA, 25 bp, synthetic construct
  source 1..25: mol_type = other DNA; note = PPMO6
  misc_feature 1..25: note = PPMO modification
tgggagacatctgaaaatggtcagg

SEQ ID NO: 151 - DNA, 26 bp, synthetic construct
  source 1..26: mol_type = other DNA; note = PPMO7
  misc_feature 1..26: note = PPMO modification
tggttgtatcttctgtacgtggacaa

SEQ ID NO: 152 - DNA, 25 bp, synthetic construct
  source 1..25: mol_type = other DNA; note = PPMO8
  misc_feature 1..25: note = PPMO modification
gactcgccattctctgtcacctgaa

SEQ ID NO: 153 - DNA, 25 bp, synthetic construct
  source 1..25: mol_type = other DNA; note = PPMO9
  misc_feature 1..25: note = PPMO modification
ttcgaaatctagtaccagagcaggt

SEQ ID NO: 154 - DNA, 69 bp, Synthetic construct
  source 1..69: mol_type = other DNA; note = Target Region Base Sequence
ttcctttgggcaggtgctatctagacctgaagtagcgggaagagcagaaaggatggggcagccatctct

SEQ ID NO: 155 - DNA, 55 bp, Synthetic construct
  source 1..55: mol_type = other DNA; note = Target Region Base Sequence
ttcctttgggcaggtgctatctagacctgaagtagcgggaagagcagaaaggatg

SEQ ID NO: 156 - DNA, 35 bp, Synthetic construct
  source 1..35: mol_type = other DNA; note = Target Region Base Sequence
gcgggaagagcagaaaggatggggcagccatctct

SEQ ID NO: 157 - DNA, 40 bp, Synthetic construct
  source 1..40: mol_type = other DNA; note = Target Region Base Sequence
catctctgttcacaggacaccagacatcagagacagagag

SEQ ID NO: 158 - DNA, 33 bp, Synthetic construct
  source 1..33: mol_type = other DNA; note = Target Region Base Sequence
ctgacttggatgctgatggtggtggtggcctct

SEQ ID NO: 159 - DNA, 25 bp, Synthetic construct
  source 1..25: mol_type = other DNA; note = Target Region Base Sequence
cctaacctgtttcagatatctccct

SEQ ID NO: 160 - DNA, 26 bp, Synthetic construct
  source 1..26: mol_type = other DNA; note = Target Region Base Sequence
tgtgggacagtgttgacggtacgtcc

SEQ ID NO: 161 - DNA, 31 bp, Synthetic construct
  source 1..31: mol_type = other DNA; note = Target Region Base Sequence
tgagatcatcatccgtgacctcaacatcaaa

SEQ ID NO: 162 - DNA, 21 bp, Synthetic construct
  source 1..21: mol_type = other DNA; note = Target Region Base Sequence
atccgtgacctcaacatcaaa

SEQ ID NO: 163 - DNA, 32 bp, Synthetic construct
  source 1..32: mol_type = other DNA; note = Target Region Base Sequence
ccctggacatgaaagtcagcctgaagaccgcc

SEQ ID NO: 164 - DNA, 22 bp, Synthetic construct
  source 1..22: mol_type = other DNA; note = Target Region Base Sequence
gaaagtcagcctgaagaccgcc

SEQ ID NO: 165 - DNA, 31 bp, Synthetic construct
  source 1..31: mol_type = other DNA; note = Target Region Base Sequence
gcctgaagaccgccctacagccaatggtcag

SEQ ID NO: 166 - DNA, 23 bp, Synthetic construct
  source 1..23: mol_type = other DNA; note = Target Region Base Sequence
gcctgaagaccgccctacagcca

SEQ ID NO: 167 - DNA, 22 bp, Synthetic construct
  source 1..22: mol_type = other DNA; note = Target Region Base Sequence
cctgaagaccgccctacagcca

SEQ ID NO: 168 - DNA, 27 bp, Synthetic construct
  source 1..27: mol_type = other DNA; note = Target Region Base Sequence
gaagaccgccctacagccaatggtcag

SEQ ID NO: 169 - DNA, 30 bp, Synthetic construct
  source 1..30: mol_type = other DNA; note = Target Region Base Sequence
gaccgccctacagccaatggtcaggtgtgg

SEQ ID NO: 170 - DNA, 21 bp, Synthetic construct
  source 1..21: mol_type = other DNA; note = Target Region Base Sequence
acagccaatggtcaggtgtgg

SEQ ID NO: 171 - DNA, 30 bp, Synthetic construct
  source 1..30: mol_type = other DNA; note = Target Region Base Sequence
cctcccagggccgattttccgtccagatgt

SEQ ID NO: 172 - DNA, 33 bp, Synthetic construct
  source 1..33: mol_type = other DNA; note = Target Region Base Sequence
tccgtccagatgttccggtttgctggaaactat

SEQ ID NO: 173 - DNA, 25 bp, Synthetic construct
  source 1..25: mol_type = other DNA; note = Target Region Base Sequence
gatgttccggtttgctggaaactat

SEQ ID NO: 174 - DNA, 25 bp, Synthetic construct
  source 1..25: mol_type = other DNA; note = Target Region Base Sequence
tccggtttgctggaaactatgacct

SEQ ID NO: 175 - DNA, 31 bp, Synthetic construct
  source 1..31: mol_type = other DNA; note = Target Region Base Sequence
aaactatgacctagtctacctgcactgtgaa

SEQ ID NO: 176 - DNA, 29 bp, Synthetic construct
  source 1..29: mol_type = other DNA; note = Target Region Base Sequence
gtgaagtctatctctgtgacaccatgaat

SEQ ID NO: 177 - DNA, 23 bp, Synthetic construct
  source 1..23: mol_type = other DNA; note = Target Region Base Sequence
tctatctctgtgacaccatgaat

SEQ ID NO: 178 - DNA, 30 bp, Synthetic construct
  source 1..30: mol_type = other DNA; note = Target Region Base Sequence
tgtagacctgctctgggaccagattccgaa

SEQ ID NO: 179 - protein, 9 aa, Synthetic construct
  source 1..9: note = Cell-Penetrating Peptide
RRRQRRKKR

SEQ ID NO: 180 - protein, 9 aa, Synthetic construct
  source 1..9: note = Cell-Penetrating Peptide
RKKRRQRRR

SEQ ID NO: 181 - protein, 11 aa, Synthetic construct
  source 1..11: note = Cell-Penetrating Peptide
RRRRRRRRRFF

SEQ ID NO: 182 - protein, 11 aa, Synthetic construct
  source 1..11: note = Cell-Penetrating Peptide
RRRRRFFRRRR

SEQ ID NO: 183 - protein, 4 aa, Synthetic construct
  source 1..4: note = Cell-Penetrating Peptide
RRRR

SEQ ID NO: 184 - protein, 5 aa, Synthetic construct
  source 1..5: note = Cell-Penetrating Peptide
RRRRR

SEQ ID NO: 185 - protein, 6 aa, Synthetic construct
  source 1..6: note = Cell-Penetrating Peptide
RRRRRR

SEQ ID NO: 186 - protein, 9 aa, Synthetic construct
  source 1..9: note = Cell-Penetrating Peptide
RFFRFFRFF

SEQ ID NO: 187 - protein, 10 aa, Synthetic construct
  source 1..10: note = Cell-Penetrating Peptide
RTRTRFLRRT

SEQ ID NO: 188 - protein, 10 aa, Synthetic construct
  source 1..10: note = Cell-Penetrating Peptide
RFFRFFRFFR

SEQ ID NO: 189 - protein, 10 aa, Synthetic construct
  source 1..10: note = Cell-Penetrating Peptide
KTRTKFLKKT

SEQ ID NO: 190 - protein, 9 aa, Synthetic construct
  source 1..9: note = Cell-Penetrating Peptide
KFFKFFKFF

SEQ ID NO: 191 - protein, 10 aa, Synthetic construct
  source 1..10: note = Cell-Penetrating Peptide
KFFKFFKFFK

SEQ ID NO: 192 - protein, 6 aa, Synthetic construct
  source 1..6: note = Cell-Penetrating Peptide
RFFRFF

SEQ ID NO: 193 - protein, 7 aa, Synthetic construct
  source 1..7: note = Cell-Penetrating Peptide
RFFRFFR

SEQ ID NO: 194 - skipped

SEQ ID NO: 195 - protein, 11 aa, Synthetic construct
  source 1..11: note = Cell-Penetrating Peptide
YGRKKRRQRRR

SEQ ID NO: 196 - protein, 10 aa, Synthetic construct
  source 1..10: note = Cell-Penetrating Peptide
GRKKRRQRRR

SEQ ID NO: 197 - protein, 18 aa, Synthetic construct
  source 1..18: note = Cell-Penetrating Peptide
RQIKIWFQNRRMKWKKGG

SEQ ID NO: 198 - protein, 27 aa, Synthetic construct
  source 1..27: note = Cell-Penetrating Peptide
GWTLNSAGYLLGKINLKALAALAKKIL

SEQ ID NO: 199 - protein, 22 aa, Synthetic construct
  source 1..22: note = Cell-Penetrating Peptide
YARVRRRGPRGYARVRRRGPRR

SEQ ID NO: 200 - protein, 11 aa, Synthetic construct
  source 1..11: note = Cell-Penetrating Peptide
YARVRRRGPRR

SEQ ID NO: 201 - protein, 9 aa, Synthetic construct
  source 1..9: note = Cell-Penetrating Peptide
AKAARQAAR

SEQ ID NO: 202 - protein, 34 aa, Synthetic construct
  source 1..34: note = Cell-Penetrating Peptide
DAATATRGRSAASRPTERPRAPARSASRPRRPVE

SEQ ID NO: 203 - protein, 21 aa, Synthetic construct
  source 1..21: note = Cell-Penetrating Peptide
KETWWETWWTEWSQPKKKRKV

SEQ ID NO: 204 - protein, 21 aa, Synthetic construct
  source 1..21: note = Cell-Penetrating Peptide
KETWFETWFTEWSQPKKKRKV

SEQ ID NO: 205 - protein, 16 aa, Synthetic construct
  source 1..16: note = Cell-Penetrating Peptide
RQIKIWFQNRRMKWKK

SEQ ID NO: 206 - protein, 24 aa, Synthetic construct
  source 1..24: note = Cell-Penetrating Peptide
RRRRRRRQIKIWFQNRRMKWKKGG

SEQ ID NO: 207 - protein, 10 aa, Synthetic construct
  source 1..10: note = Cell-Penetrating Peptide
RRRQRRKKRC

SEQ ID NO: 208 - protein, 12 aa, Synthetic construct
  source 1..12: note = Cell-Penetrating Peptide
CYGRKKRRQRRR

SEQ ID NO: 209 - protein, 12 aa, Synthetic construct
  source 1..12: note = Cell-Penetrating Peptide
RRRRRRRRRFFC

SEQ ID NO: 210 - protein, 12 aa, Synthetic construct
  source 1..12: note = Cell-Penetrating Peptide
RRRRRRRRRCFF

SEQ ID NO: 211 - protein, 12 aa, Synthetic construct
  source 1..12: note = Cell-Penetrating Peptide
RRRRRRRRCFFR

SEQ ID NO: 212 - protein, 12 aa, Synthetic construct
  source 1..12: note = Cell-Penetrating Peptide
RRRRRRCFFRRR

SEQ ID NO: 213 - protein, 12 aa, Synthetic construct
  source 1..12: note = Cell-Penetrating Peptide
RRRRRFCFRRRR

SEQ ID NO: 214 - protein, 12 aa, Synthetic construct
  source 1..12: note = Cell-Penetrating Peptide
RRRRCFFRRRRR

SEQ ID NO: 215 - protein, 12 aa, Synthetic construct
  source 1..12: note = Cell-Penetrating Peptide
RRCFFRRRRRRR

SEQ ID NO: 216 - protein, 12 aa, Synthetic construct
  source 1..12: note = Cell-Penetrating Peptide
CFFRRRRRRRRR

SEQ ID NO: 217 - protein, 12 aa, Synthetic construct
  source 1..12: note = Cell-Penetrating Peptide
CRRRRRRRRRFF

SEQ ID NO: 218 - protein, 11 aa, Synthetic construct
  source 1..11: note = Cell-Penetrating Peptide
FFRRRRRRRRR

SEQ ID NO: 219 - protein, 14 aa, Synthetic construct
  source 1..14: note = Cell-Penetrating Peptide
RRRRRFFCFFRRRR

SEQ ID NO: 220 - protein, 11 aa, Synthetic construct
  source 1..11: note = Cell-Penetrating Peptide
RRRRRRRRRII

SEQ ID NO: 221 - protein, 11 aa, Synthetic construct
  source 1..11: note = Cell-Penetrating Peptide
RRRRRRRRFFF

SEQ ID NO: 222 - protein, 13 aa, Synthetic construct
  source 1..13: note = Cell-Penetrating Peptide
RRRRRRRRRFFFF

SEQ ID NO: 223 - protein, 10 aa, Synthetic construct
  source 1..10: note = Cell-Penetrating Peptide
RRRRRRRRFF

SEQ ID NO: 224 - protein, 8 aa, Synthetic construct
  source 1..8: note = Cell-Penetrating Peptide
RRRRRRFF

SEQ ID NO: 225 - protein, 7 aa, Synthetic construct
  source 1..7: note = Cell-Penetrating Peptide
RRRRRFF

SEQ ID NO: 226 - protein, 11 aa, Synthetic construct
  source 1..11: note = Cell-Penetrating Peptide
  MOD_RES 3: note = 6-aminohexanoic acid
  MOD_RES 6: note = 6-aminohexanoic acid
  MOD_RES 9: note = 6-aminohexanoic acid
RRXRRXRRXRR

SEQ ID NO: 227 - protein, 12 aa, Synthetic construct
  source 1..12: note = Cell-Penetrating Peptide
  MOD_RES 1: note = 6-aminohexanoic acid
  MOD_RES 4: note = 6-aminohexanoic acid
  MOD_RES 7: note = 6-aminohexanoic acid
  MOD_RES 10: note = 6-aminohexanoic acid
XRRXRRXRRXRR

SEQ ID NO: 228 - protein, 11 aa, Synthetic construct
  source 1..11: note = Cell-Penetrating Peptide
  MOD_RES 2: note = 6-aminohexanoic acid
  MOD_RES 4: note = 6-aminohexanoic acid
  MOD_RES 6: note = 6-aminohexanoic acid
  MOD_RES 8: note = 6-aminohexanoic acid
  MOD_RES 10: note = 6-aminohexanoic acid
RXRXRXRXRXR

SEQ ID NO: 229 - protein, 9 aa, Synthetic construct
  source 1..9: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXR

SEQ ID NO: 230 - protein, 7 aa, Synthetic construct
  source 1..7: note = Cell-Penetrating Peptide
  MOD_RES 2: note = 6-aminohexanoic acid
  MOD_RES 5: note = 6-aminohexanoic acid
RXRRXRR

SEQ ID NO: 231 - protein, 6 aa, Synthetic construct
  source 1..6: note = Cell-Penetrating Peptide
  MOD_RES 2: note = 6-aminohexanoic acid
  MOD_RES 5: note = 6-aminohexanoic acid
RXRRXR

SEQ ID NO: 232 - protein, 11 aa, Synthetic construct
  source 1..11: note = Cell-Penetrating Peptide
  MOD_RES 3: note = 6-aminohexanoic acid
  MOD_RES 6: note = 6-aminohexanoic acid
  MOD_RES 9: note = 6-aminohexanoic acid
RKXRKXRKXRK

SEQ ID NO: 233 - protein, 11 aa, Synthetic construct
  source 1..11: note = Cell-Penetrating Peptide
  MOD_RES 3: note = 6-aminohexanoic acid
  MOD_RES 6: note = 6-aminohexanoic acid
  MOD_RES 9: note = 6-aminohexanoic acid
RHXRHXRHXRH

SEQ ID NO: 234 - protein, 13 aa, Synthetic construct
  source 1..13: note = Cell-Penetrating Peptide
  MOD_RES 2: note = 6-aminohexanoic acid
  MOD_RES 4: note = 6-aminohexanoic acid
  MOD_RES 6: note = 6-aminohexanoic acid
  MOD_RES 8: note = 6-aminohexanoic acid
  MOD_RES 10: note = 6-aminohexanoic acid
  MOD_RES 12: note = 6-aminohexanoic acid
RXRXRXRXRXRXR

SEQ ID NO: 235 - protein, 12 aa, Synthetic construct
  source 1..12: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = b-Ala
RXRRXRRXRRBR

SEQ ID NO: 236 - protein, 15 aa, Synthetic construct
  source 1..15: note = Cell-Penetrating Peptide
  MOD_RES 2: note = b-Ala
  MOD_RES 4: note = b-Ala
  MOD_RES 6: note = b-Ala
  MOD_RES 8: note = b-Ala
  MOD_RES 10: note = b-Ala
  MOD_RES 14: note = b-Ala
  MOD_RES 12: note = 6-aminohexanoic acid
RBRBRBRBRBRXRBR

SEQ ID NO: 237 - protein, 15 aa, Synthetic construct
  source 1..15: note = Cell-Penetrating Peptide
  MOD_RES 8: note = 6-aminohexanoic acid
  MOD_RES 2: note = b-Ala
  MOD_RES 4: note = b-Ala
  MOD_RES 6: note = b-Ala
  MOD_RES 10: note = b-Ala
  MOD_RES 12: note = b-Ala
  MOD_RES 14: note = b-Ala
RBRBRBRXRBRBRBR

SEQ ID NO: 238 - protein, 17 aa, Synthetic construct
  source 1..17: note = Cell-Penetrating Peptide
  MOD_RES 3: note = b-Ala
  MOD_RES 5: note = b-Ala
  MOD_RES 7: note = b-Ala
  MOD_RES 11: note = b-Ala
  MOD_RES 13: note = b-Ala
  MOD_RES 15: note = b-Ala
  MOD_RES 1: note = 6-aminohexanoic acid
  MOD_RES 9: note = 6-aminohexanoic acid
  MOD_RES 17: note = 6-aminohexanoic acid
XRBRBRBRXRBRBRBRX

SEQ ID NO: 239 - protein, 16 aa, Synthetic construct
  source 1..16: note = Cell-Penetrating Peptide
  MOD_RES 4: note = 6-aminohexanoic acid
  MOD_RES 8: note = 6-aminohexanoic acid
  MOD_RES 2: note = b-Ala
  MOD_RES 6: note = b-Ala
  MOD_RES 10: note = b-Ala
  MOD_RES 14: note = b-Ala
  MOD_RES 12: note = 6-aminohexanoic acid
  MOD_RES 16: note = 6-aminohexanoic acid
RBRXRBRXRBRXRBRX

SEQ ID NO: 240 - protein, 15 aa, Synthetic construct
  source 1..15: note = Cell-Penetrating Peptide
  MOD_RES 10: note = 6-aminohexanoic acid
  MOD_RES 12: note = 6-aminohexanoic acid
  MOD_RES 14: note = 6-aminohexanoic acid
  MOD_RES 2: note = b-Ala
  MOD_RES 4: note = 6-aminohexanoic acid
  MOD_RES 6: note = 6-aminohexanoic acid
  MOD_RES 8: note = 6-aminohexanoic acid
RBRBRBRBRXRXRXR

SEQ ID NO: 241 - protein, 15 aa, Synthetic construct
  source 1..15: note = Cell-Penetrating Peptide
  MOD_RES 2: note = 6-aminohexanoic acid
  MOD_RES 8: note = 6-aminohexanoic acid
  MOD_RES 4: note = b-Ala
  MOD_RES 6: note = b-Ala
  MOD_RES 10: note = b-Ala
  MOD_RES 12: note = b-Ala
  MOD_RES 14: note = b-Ala
RXRBRBRXRBRBRBR

SEQ ID NO: 242 - protein, 15 aa, Synthetic construct
  source 1..15: note = Cell-Penetrating Peptide
  MOD_RES 2: note = b-Ala
  MOD_RES 4: note = b-Ala
  MOD_RES 6: note = b-Ala
  MOD_RES 8: note = b-Ala
  MOD_RES 10: note = b-Ala
  MOD_RES 12: note = b-Ala
  MOD_RES 14: note = b-Ala
RBRBRBRBRBRBRBR

SEQ ID NO: 243 - skipped

SEQ ID NO: 244 - protein, 10 aa, Synthetic construct
  source 1..10: note = Cell-Penetrating Peptide
RRRRRGRRRR

SEQ ID NO: 245 - protein, 6 aa, Synthetic construct
  source 1..6: note = Cell-Penetrating Peptide
  MOD_RES 2: note = b-Ala
  MOD_RES 5: note = b-Ala
RBRRBR

SEQ ID NO: 246 - protein, 6 aa, Synthetic construct
  source 1..6: note = Cell-Penetrating Peptide
  MOD_RES 5: note = b-Ala
  MOD_RES 2: note = 6-aminohexanoic acid
RXRRBR

SEQ ID NO: 247 - protein, 6 aa, Synthetic construct
  source 1..6: note = Cell-Penetrating Peptide
  MOD_RES 5: note = 6-aminohexanoic acid
  MOD_RES 2: note = b-Ala
RBRRXR

SEQ ID NO: 248 - protein, 8 aa, Synthetic construct
  source 1..8: note = Cell-Penetrating Peptide
  MOD_RES 2: note = 6-aminohexanoic acid
  MOD_RES 7: note = 6-aminohexanoic acid
  MOD_RES 5: note = b-Ala
RXRYBRXR

SEQ ID NO: 249 - protein, 8 aa, Synthetic construct
  source 1..8: note = Cell-Penetrating Peptide
  MOD_RES 2: note = b-Ala
  MOD_RES 5: note = b-Ala
  MOD_RES 7: note = b-Ala
RBRYBRBR

SEQ ID NO: 250 - protein, 8 aa, Synthetic construct
  source 1..8: note = Cell-Penetrating Peptide
  MOD_RES 5: note = b-Ala
  MOD_RES 7: note = b-Ala
  MOD_RES 2: note = 6-aminohexanoic acid
RXRYBRBR

SEQ ID NO: 251 - protein, 8 aa, Synthetic construct
  source 1..8: note = Cell-Penetrating Peptide
  MOD_RES 7: note = 6-aminohexanoic acid
  MOD_RES 2: note = b-Ala
  MOD_RES 5: note = b-Ala
RBRYBRXR

SEQ ID NO: 252 - protein, 11 aa, Synthetic construct
  source 1..11: note = Cell-Penetrating Peptide
  MOD_RES 2: note = 6-aminohexanoic acid
  MOD_RES 10: note = 6-aminohexanoic acid
RXRILFQYRXR

SEQ ID NO: 253 - protein, 11 aa, Synthetic construct
  source 1..11: note = Cell-Penetrating Peptide
  MOD_RES 2: note = b-Ala
  MOD_RES 10: note = b-Ala
RBRILFQYRBR

SEQ ID NO: 254 - protein, 11 aa, Synthetic construct
  source 1..11: note = Cell-Penetrating Peptide
  MOD_RES 2: note = 6-aminohexanoic acid
  MOD_RES 10: note = b-Ala
RXRILFQYRBR

SEQ ID NO: 255 - protein, 11 aa, Synthetic construct
  source 1..11: note = Cell-Penetrating Peptide
  MOD_RES 10: note = 6-aminohexanoic acid
  MOD_RES 2: note = b-Ala
RBRILFQYRXR

SEQ ID NO: 256 - protein, 9 aa, Synthetic construct
  source 1..9: note = Cell-Penetrating Peptide
  MOD_RES 2: note = b-Ala
  MOD_RES 5: note = b-Ala
  MOD_RES 8: note = b-Ala
RBRRBRRBR

SEQ ID NO: 257 - protein, 9 aa, Synthetic construct
  source 1..9: note = Cell-Penetrating Peptide
  MOD_RES 2: note = 6-aminohexanoic acid
  MOD_RES 8: note = 6-aminohexanoic acid
  MOD_RES 5: note = b-Ala
RXRRBRRXR

SEQ ID NO: 258 - protein, 9 aa, Synthetic construct
  source 1..9: note = Cell-Penetrating Peptide
  MOD_RES 2: note = 6-aminohexanoic acid
  MOD_RES 5: note = b-Ala
  MOD_RES 8: note = b-Ala
RXRRBRRBR

SEQ ID NO: 259 - protein, 9 aa, Synthetic construct
  source 1..9: note = Cell-Penetrating Peptide
  MOD_RES 2: note = 6-aminohexanoic acid
  MOD_RES 5: note = 6-aminohexanoic acid
  MOD_RES 8: note = b-Ala
RXRRXRRBR

SEQ ID NO: 260 - protein, 9 aa, Synthetic construct
  source 1..9: note = Cell-Penetrating Peptide
  MOD_RES 5: note = 6-aminohexanoic acid
  MOD_RES 2: note = b-Ala
  MOD_RES 8: note = b-Ala
RBRRXRRBR

SEQ ID NO: 261 - protein, 9 aa, Synthetic construct
  source 1..9: note = Cell-Penetrating Peptide
  MOD_RES 5: note = 6-aminohexanoic acid
  MOD_RES 8: note = 6-aminohexanoic acid
  MOD_RES 2: note = b-Ala
RBRRXRRXR

SEQ ID NO: 262 - protein, 9 aa, Synthetic construct
  source 1..9: note = Cell-Penetrating Peptide
  MOD_RES 2: note = b-Ala
  MOD_RES 5: note = b-Ala
  MOD_RES 8: note = 6-aminohexanoic acid
RBRRBRRXR

SEQ ID NO: 263 - protein, 11 aa, Synthetic construct
  source 1..11: note = Cell-Penetrating Peptide
  MOD_RES 2: note = 6-aminohexanoic acid
  MOD_RES 7: note = 6-aminohexanoic acid
  MOD_RES 10: note = 6-aminohexanoic acid
  MOD_RES 5: note = b-Ala
RXRYBRXRRXR

SEQ ID NO: 264 - protein, 11 aa, Synthetic construct
  source 1..11: note = Cell-Penetrating Peptide
  MOD_RES 2: note = 6-aminohexanoic acid
  MOD_RES 5: note = 6-aminohexanoic acid
  MOD_RES 10: note = 6-aminohexanoic acid
  MOD_RES 8: note = b-Ala
RXRRXRYBRXR

SEQ ID NO: 265 - protein, 14 aa, Synthetic construct
  source 1..14: note = Cell-Penetrating Peptide
  MOD_RES 2: note = 6-aminohexanoic acid
  MOD_RES 10: note = 6-aminohexanoic acid
  MOD_RES 13: note = 6-aminohexanoic acid
RXRILFQYRXRRXR

SEQ ID NO: 266 - protein, 14 aa, Synthetic construct
  source 1..14: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
RXRRXRILFQYRXR

SEQ ID NO: 267 - protein, 13 aa, Synthetic construct
  source 1..13: note = Cell-Penetrating Peptide
  MOD_RES 2: note = 6-aminohexanoic acid
  MOD_RES 12: note = 6-aminohexanoic acid
  MOD_RES 7: note = 6-aminohexanoic acid
  MOD_RES 10: note = b-Ala
  MOD_RES 5: note = b-Ala
RXRYBRXRYBRXR

SEQ ID NO: 268 - protein, 19 aa, Synthetic construct
  source 1..19: note = Cell-Penetrating Peptide
  MOD_RES 2: note = 6-aminohexanoic acid
  MOD_RES 10: note = 6-aminohexanoic acid
  MOD_RES 18: note = 6-aminohexanoic acid
RXRILFQYRXRILFQYRXR

SEQ ID NO: 269 - protein, 16 aa, Synthetic construct
  source 1..16: note = Cell-Penetrating Peptide
  MOD_RES 2: note = 6-aminohexanoic acid
  MOD_RES 10: note = 6-aminohexanoic acid
  MOD_RES 15: note = 6-aminohexanoic acid
  MOD_RES 13: note = b-Ala
RXRILFQYRXRYBRXR

SEQ ID NO: 270 - protein, 16 aa, Synthetic construct
  source 1..16: note = Cell-Penetrating Peptide
  MOD_RES 2: note = 6-aminohexanoic acid
  MOD_RES 7: note = 6-aminohexanoic acid
  MOD_RES 15: note = 6-aminohexanoic acid
  MOD_RES 5: note = b-Ala
RXRYBRXRILFQYRXR

SEQ ID NO: 271 - protein, 11 aa, Synthetic construct
  source 1..11: note = Cell-Penetrating Peptide
  MOD_RES 2: note = b-Ala
  MOD_RES 5: note = b-Ala
  MOD_RES 7: note = b-Ala
  MOD_RES 10: note = b-Ala
RBRYBRBRRBR

SEQ ID NO: 272 - protein, 11 aa, Synthetic construct
  source 1..11: note = Cell-Penetrating Peptide
  MOD_RES 2: note = b-Ala
  MOD_RES 5: note = b-Ala
  MOD_RES 8: note = b-Ala
  MOD_RES 10: note = b-Ala
RBRRBRYBRBR

SEQ ID NO: 273 - protein, 14 aa, Synthetic construct
  source 1..14: note = Cell-Penetrating Peptide
  MOD_RES 2: note = b-Ala
  MOD_RES 10: note = b-Ala
  MOD_RES 13: note = b-Ala
RBRILFQYRBRRBR

SEQ ID NO: 274 - protein, 14 aa, Synthetic construct
  source 1..14: note = Cell-Penetrating Peptide
  MOD_RES 2: note = b-Ala
  MOD_RES 5: note = b-Ala
  MOD_RES 13: note = b-Ala
RBRRBRILFQYRBR

SEQ ID NO: 275 - protein, 12 aa, Synthetic construct
  source 1..12: note = Cell-Penetrating Peptide
  MOD_RES 2: note = b-Ala
  MOD_RES 11: note = b-Ala
  MOD_RES 6: note = b-Ala
  MOD_RES 9: note = b-Ala
RBRYRBRYBRBR

SEQ ID NO: 276 - protein, 19 aa, Synthetic construct
  source 1..19: note = Cell-Penetrating Peptide
  MOD_RES 2: note = b-Ala
  MOD_RES 18: note = b-Ala
  MOD_RES 10: note = b-Ala
RBRILFQYRBRILFQYRBR

SEQ ID NO: 277 - protein, 16 aa, Synthetic construct
  source 1..16: note = Cell-Penetrating Peptide
  MOD_RES 2: note = b-Ala
  MOD_RES 15: note = b-Ala
  MOD_RES 5: note = b-Ala
  MOD_RES 7: note = b-Ala
RBRYBRBRILFQYRBR

SEQ ID NO: 278 - protein, 16 aa, Synthetic construct
  source 1..16: note = Cell-Penetrating Peptide
  MOD_RES 2: note = b-Ala
  MOD_RES 15: note = b-Ala
  MOD_RES 13: note = b-Ala
  MOD_RES 10: note = b-Ala
RBRILFQYRBRYBRBR

SEQ ID NO: 279 - protein, 11 aa, Synthetic construct
  source 1..11: note = Cell-Penetrating Peptide
  MOD_RES 5: note = b-Ala
  MOD_RES 7: note = b-Ala
  MOD_RES 2: note = 6-aminohexanoic acid
  MOD_RES 10: note = 6-aminohexanoic acid
RXRYBRBRRXR

SEQ ID NO: 280 - protein, 11 aa, Synthetic construct
  source 1..11: note = Cell-Penetrating Peptide
  MOD_RES 2: note = 6-aminohexanoic acid
  MOD_RES 10: note = 6-aminohexanoic acid
  MOD_RES 5: note = b-Ala
  MOD_RES 8: note = b-Ala
RXRRBRYBRXR

SEQ ID NO: 281 - protein, 14 aa, Synthetic construct
  source 1..14: note = Cell-Penetrating Peptide
  MOD_RES 13: note = 6-aminohexanoic acid
  MOD_RES 2: note = 6-aminohexanoic acid
  MOD_RES 10: note = b-Ala
RXRILFQYRBRRXR

SEQ ID NO: 282 - protein, 14 aa, Synthetic construct
  source 1..14: note = Cell-Penetrating Peptide
  MOD_RES 2: note = 6-aminohexanoic acid
  MOD_RES 13: note = 6-aminohexanoic acid
  MOD_RES 5: note = b-Ala
RXRRBRILFQYRXR

SEQ ID NO: 283 - protein, 13 aa, Synthetic construct
  source 1..13: note = Cell-Penetrating Peptide
  MOD_RES 2: note = 6-aminohexanoic acid
  MOD_RES 12: note = 6-aminohexanoic acid
  MOD_RES 5: note = b-Ala
  MOD_RES 7: note = b-Ala
  MOD_RES 10: note = b-Ala
RXRYBRBRYBRXR

SEQ ID NO: 284 - protein, 19 aa, Synthetic construct
  source 1..19: note = Cell-Penetrating Peptide
  MOD_RES 2: note = 6-aminohexanoic acid
  MOD_RES 18: note = 6-aminohexanoic acid
  MOD_RES 10: note = b-Ala
RXRILFQYRBRILFQYRXR

SEQ ID NO: 285 - protein, 16 aa, Synthetic construct
  source 1..16: note = Cell-Penetrating Peptide
  MOD_RES 2: note = 6-aminohexanoic acid
  MOD_RES 15: note = 6-aminohexanoic acid
  MOD_RES 5: note = b-Ala
  MOD_RES 7: note = b-Ala
RXRYBRBRILFQYRXR

SEQ ID NO: 286 - protein, 16 aa, Synthetic construct
  source 1..16: note = Cell-Penetrating Peptide
  MOD_RES 2: note = 6-aminohexanoic acid
  MOD_RES 15: note = 6-aminohexanoic acid
  MOD_RES 10: note = b-Ala
  MOD_RES 13: note = b-Ala
RXRILFQYRBRYBRXR

SEQ ID NO: 287 - protein, 11 aa, Synthetic construct
  source 1..11: note = Cell-Penetrating Peptide
  MOD_RES 2: note = 6-aminohexanoic acid
  MOD_RES 5: note = b-Ala
  MOD_RES 7: note = b-Ala
  MOD_RES 10: note = b-Ala
RXRYBRBRRBR

SEQ ID NO: 288 - protein, 11 aa, Synthetic construct
  source 1..11: note = Cell-Penetrating Peptide
  MOD_RES 2: note = 6-aminohexanoic acid
  MOD_RES 5: note = b-Ala
  MOD_RES 8: note = b-Ala
  MOD_RES 10: note = b-Ala
RXRRBRYBRBR

SEQ ID NO: 289 - protein, 14 aa, Synthetic construct
  source 1..14: note = Cell-Penetrating Peptide
  MOD_RES 2: note = 6-aminohexanoic acid
  MOD_RES 10: note = b-Ala
  MOD_RES 13: note = b-Ala
RXRILFQYRBRRBR

SEQ ID NO: 290 - protein, 14 aa, Synthetic construct
  source 1..14: note = Cell-Penetrating Peptide
  MOD_RES 2: note = 6-aminohexanoic acid
  MOD_RES 5: note = b-Ala
  MOD_RES 13: note = b-Ala
RXRRBRILFQYRBR

SEQ ID NO: 291 - protein, 13 aa, Synthetic construct
  source 1..13: note = Cell-Penetrating Peptide
  MOD_RES 2: note = 6-aminohexanoic acid
  MOD_RES 12: note = b-Ala
  MOD_RES 5: note = b-Ala
  MOD_RES 7: note = b-Ala
  MOD_RES 10: note = b-Ala
RXRYBRBRYBRBR

SEQ ID NO: 292 - protein, 19 aa, Synthetic construct
  source 1..19: note = Cell-Penetrating Peptide
  MOD_RES 2: note = 6-aminohexanoic acid
  MOD_RES 18: note = b-Ala
  MOD_RES 10: note = b-Ala
RXRILFQYRBRILFQYRBR

SEQ ID NO: 293 - protein, 16 aa, Synthetic construct
  source 1..16: note = Cell-Penetrating Peptide
  MOD_RES 15: note = b-Ala
  MOD_RES 5: note = b-Ala
  MOD_RES 7: note = b-Ala
  MOD_RES 2: note = 6-aminohexanoic acid
RXRYBRBRILFQYRBR

SEQ ID NO: 294 - protein, 16 aa, Synthetic construct
  source 1..16: note = Cell-Penetrating Peptide
  MOD_RES 2: note = 6-aminohexanoic acid
  MOD_RES 15: note = b-Ala
  MOD_RES 10: note = b-Ala
  MOD_RES 13: note = b-Ala
RXRILFQYRBRYBRBR

SEQ ID NO: 295 - protein, 11 aa, Synthetic construct
  source 1..11: note = Cell-Penetrating Peptide
  MOD_RES 2: note = 6-aminohexanoic acid
  MOD_RES 7: note = 6-aminohexanoic acid
  MOD_RES 5: note = b-Ala
  MOD_RES 10: note = b-Ala
RXRYBRXRRBR

SEQ ID NO: 296 - protein, 11 aa, Synthetic construct
  source 1..11: note = Cell-Penetrating Peptide
  MOD_RES 2: note = 6-aminohexanoic acid
  MOD_RES 5: note = 6-aminohexanoic acid
  MOD_RES 8: note = b-Ala
  MOD_RES 10: note = b-Ala
RXRRXRYBRBR

SEQ ID NO: 297 - protein, 14 aa, Synthetic construct
  source 1..14: note = Cell-Penetrating Peptide
  MOD_RES 13: note = b-Ala
  MOD_RES 2: note = 6-aminohexanoic acid
  MOD_RES 10: note = 6-aminohexanoic acid
RXRILFQYRXRRBR

SEQ ID NO: 298 - protein, 14 aa, Synthetic construct
  source 1..14: note = Cell-Penetrating Peptide
  MOD_RES 13: note = b-Ala
  MOD_RES 2: note = 6-aminohexanoic acid
  MOD_RES 5: note = 6-aminohexanoic acid
RXRRXRILFQYRBR

SEQ ID NO: 299 - protein, 13 aa, Synthetic construct
  source 1..13: note = Cell-Penetrating Peptide
  MOD_RES 2: note = 6-aminohexanoic acid
  MOD_RES 7: note = 6-aminohexanoic acid
  MOD_RES 12: note = b-Ala
  MOD_RES 5: note = b-Ala
  MOD_RES 10: note = b-Ala
RXRYBRXRYBRBR

SEQ ID NO: 300 - protein, 19 aa, Synthetic construct
  source 1..19: note = Cell-Penetrating Peptide
  MOD_RES 18: note = b-Ala
  MOD_RES 2: note = 6-aminohexanoic acid
  MOD_RES 10: note = 6-aminohexanoic acid
RXRILFQYRXRILFQYRBR

SEQ ID NO: 301 - protein, 16 aa, Synthetic construct
  source 1..16: note = Cell-Penetrating Peptide
  MOD_RES 2: note = 6-aminohexanoic acid
  MOD_RES 7: note = 6-aminohexanoic acid
  MOD_RES 15: note = b-Ala
  MOD_RES 5: note = b-Ala
RXRYBRXRILFQYRBR

SEQ ID NO: 302 - protein, 16 aa, Synthetic construct
  source 1..16: note = Cell-Penetrating Peptide
  MOD_RES 2: note = 6-aminohexanoic acid
  MOD_RES 10: note = 6-aminohexanoic acid
  MOD_RES 15: note = b-Ala
  MOD_RES 13: note = b-Ala
RXRILFQYRXRYBRBR

SEQ ID NO: 303 - protein, 11 aa, Synthetic construct
  source 1..11: note = Cell-Penetrating Peptide
  MOD_RES 10: note = b-Ala
  MOD_RES 2: note = b-Ala
  MOD_RES 5: note = b-Ala
  MOD_RES 7: note = 6-aminohexanoic acid
RBRYBRXRRBR

SEQ ID NO: 304 - protein, 11 aa, Synthetic construct
  source 1..11: note = Cell-Penetrating Peptide
  MOD_RES 10: note = b-Ala
  MOD_RES 2: note = b-Ala
  MOD_RES 8: note = b-Ala
  MOD_RES 5: note = 6-aminohexanoic acid
RBRRXRYBRBR

SEQ ID NO: 305 - protein, 14 aa, Synthetic construct
  source 1..14: note = Cell-Penetrating Peptide
  MOD_RES 10: note = 6-aminohexanoic acid
  MOD_RES 13: note = b-Ala
  MOD_RES 2: note = b-Ala
RBRILFQYRXRRBR

SEQ ID NO: 306 - protein, 14 aa, Synthetic construct
  source 1..14: note = Cell-Penetrating Peptide
  MOD_RES 13: note = b-Ala
  MOD_RES 2: note = b-Ala
  MOD_RES 5: note = 6-aminohexanoic acid
RBRRXRILFQYRBR

SEQ ID NO: 307 - protein, 13 aa, Synthetic construct
  source 1..13: note = Cell-Penetrating Peptide
  MOD_RES 10: note = b-Ala
  MOD_RES 12: note = b-Ala
  MOD_RES 2: note = b-Ala
  MOD_RES 5: note = b-Ala
  MOD_RES 7: note = 6-aminohexanoic acid
RBRYBRXRYBRBR

SEQ ID NO: 308 - protein, 19 aa, Synthetic construct
  source 1..19: note = Cell-Penetrating Peptide
  MOD_RES 2: note = b-Ala
  MOD_RES 18: note = b-Ala
  MOD_RES 10: note = 6-aminohexanoic acid
RBRILFQYRXRILFQYRBR

SEQ ID NO: 309 - protein, 16 aa, Synthetic construct
  source 1..16: note = Cell-Penetrating Peptide
  MOD_RES 15: note = b-Ala
  MOD_RES 2: note = b-Ala
  MOD_RES 5: note = b-Ala
  MOD_RES 7: note = 6-aminohexanoic acid
RBRYBRXRILFQYRBR

SEQ ID NO: 310 - protein, 16 aa, Synthetic construct
  source 1..16: note = Cell-Penetrating Peptide
  MOD_RES 15: note = b-Ala
  MOD_RES 2: note = b-Ala
  MOD_RES 13: note = b-Ala
  MOD_RES 10: note = 6-aminohexanoic acid
RBRILFQYRXRYBRBR

SEQ ID NO: 311 - protein, 11 aa, Synthetic construct
  source 1..11: note = Cell-Penetrating Peptide
  MOD_RES 7: note = 6-aminohexanoic acid
  MOD_RES 10: note = 6-aminohexanoic acid
  MOD_RES 2: note = b-Ala
  MOD_RES 5: note = b-Ala
RBRYBRXRRXR

SEQ ID NO: 312 - protein, 11 aa, Synthetic construct
  source 1..11: note = Cell-Penetrating Peptide
  MOD_RES 5: note = 6-aminohexanoic acid
  MOD_RES 10: note = 6-aminohexanoic acid
  MOD_RES 2: note = b-Ala
  MOD_RES 8: note = b-Ala
RBRRXRYBRXR

SEQ ID NO: 313 - protein, 14 aa, Synthetic construct
  source 1..14: note = Cell-Penetrating Peptide
  MOD_RES 10: note = 6-aminohexanoic acid
  MOD_RES 13: note = 6-aminohexanoic acid
  MOD_RES 2: note = b-Ala
RBRILFQYRXRRXR

SEQ ID NO: 314 - protein, 14 aa, Synthetic construct
  source 1..14: note = Cell-Penetrating Peptide
  MOD_RES 13: note = 6-aminohexanoic acid
  MOD_RES 5: note = 6-aminohexanoic acid
  MOD_RES 2: note = b-Ala
RBRRXRILFQYRXR

SEQ ID NO: 315 - protein, 13 aa, Synthetic construct
  source 1..13: note = Cell-Penetrating Peptide
  MOD_RES 12: note = 6-aminohexanoic acid
  MOD_RES 7: note = 6-aminohexanoic acid
  MOD_RES 10: note = b-Ala
  MOD_RES 2: note = b-Ala
  MOD_RES 5: note = b-Ala
RBRYBRXRYBRXR

SEQ ID NO: 316 - protein, 19 aa, Synthetic construct
  source 1..19: note = Cell-Penetrating Peptide
  MOD_RES 18: note = 6-aminohexanoic acid
  MOD_RES 10: note = 6-aminohexanoic acid
  MOD_RES 2: note = b-Ala
RBRILFQYRXRILFQYRXR

SEQ ID NO: 317 - protein, 16 aa, Synthetic construct
  source 1..16: note = Cell-Penetrating Peptide
  MOD_RES 15: note = 6-aminohexanoic acid
  MOD_RES 7: note = 6-aminohexanoic acid
  MOD_RES 2: note = b-Ala
  MOD_RES 5: note = b-Ala
RBRYBRXRILFQYRXR

SEQ ID NO: 318 - protein, 16 aa, Synthetic construct
  source 1..16: note = Cell-Penetrating Peptide
  MOD_RES 15: note = 6-aminohexanoic acid
  MOD_RES 10: note = 6-aminohexanoic acid
  MOD_RES 2: note = b-Ala
  MOD_RES 13: note = b-Ala
RBRILFQYRXRYBRXR

SEQ ID NO: 319 - protein, 11 aa, Synthetic construct
  source 1..11: note = Cell-Penetrating Peptide
  MOD_RES 10: note = 6-aminohexanoic acid
  MOD_RES 2: note = b-Ala
  MOD_RES 5: note = b-Ala
  MOD_RES 7: note = b-Ala
RBRYBRBRRXR

SEQ ID NO: 320 - protein, 11 aa, Synthetic construct
  source 1..11: note = Cell-Penetrating Peptide
  MOD_RES 10: note = 6-aminohexanoic acid
  MOD_RES 2: note = b-Ala
  MOD_RES 5: note = b-Ala
  MOD_RES 8: note = b-Ala
RBRRBRYBRXR

SEQ ID NO: 321 - protein, 14 aa, Synthetic construct
  source 1..14: note = Cell-Penetrating Peptide
  MOD_RES 2: note = b-Ala
  MOD_RES 10: note = b-Ala
  MOD_RES 13: note = 6-aminohexanoic acid
RBRILFQYRBRRXR

SEQ ID NO: 322 - protein, 14 aa, Synthetic construct
  source 1..14: note = Cell-Penetrating Peptide
  MOD_RES 2: note = b-Ala
  MOD_RES 5: note = b-Ala
  MOD_RES 13: note = 6-aminohexanoic acid
RBRRBRILFQYRXR

SEQ ID NO: 323 - protein, 13 aa, Synthetic construct
  source 1..13: note = Cell-Penetrating Peptide
  MOD_RES 12: note = 6-aminohexanoic acid
  MOD_RES 2: note = b-Ala
  MOD_RES 5: note = b-Ala
  MOD_RES 7: note = b-Ala
  MOD_RES 10: note = b-Ala
RBRYBRBRYBRXR

SEQ ID NO: 324 - protein, 19 aa, Synthetic construct
  source 1..19: note = Cell-Penetrating Peptide
  MOD_RES 18: note = 6-aminohexanoic acid
  MOD_RES 2: note = b-Ala
  MOD_RES 10: note = b-Ala
RBRILFQYRBRILFQYRXR

SEQ ID NO: 325 - protein, 16 aa, Synthetic construct
  source 1..16: note = Cell-Penetrating Peptide
  MOD_RES 15: note = 6-aminohexanoic acid
  MOD_RES 2: note = b-Ala
  MOD_RES 5: note = b-Ala
  MOD_RES 7: note = b-Ala
RBRYBRBRILFQYRXR

SEQ ID NO: 326 - protein, 16 aa, Synthetic construct
  source 1..16: note = Cell-Penetrating Peptide
  MOD_RES 15: note = 6-aminohexanoic acid
  MOD_RES 2: note = b-Ala
  MOD_RES 10: note = b-Ala
  MOD_RES 13: note = b-Ala
RBRILFQYRBRYBRXR

SEQ ID NO: 327 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 20: note = 6-aminohexanoic acid
  MOD_RES 5: note = b-Ala
  MOD_RES 18: note = b-Ala
RXRRBRRXRILFQYRXRBRXR

SEQ ID NO: 328 - protein, 12 aa, Synthetic construct
  source 1..12: note = Cell-Penetrating Peptide
YGRKKRRQRRRP

SEQ ID NO: 329 - protein, 23 aa, Synthetic construct
  source 1..23: note = Cell-Penetrating Peptide
  MOD_RES 22: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 13: note = 6-aminohexanoic acid
  MOD_RES 14: note = b-Ala
  MOD_RES 2: note = 6-aminohexanoic acid
RXRRXRRXRRXRXBASSLNIAXC

SEQ ID NO: 330 - protein, 31 aa, Synthetic construct
  source 1..31: note = Cell-Penetrating Peptide
  MOD_RES 2: note = 6-aminohexanoic acid
  MOD_RES 30: note = 6-aminohexanoic acid
  MOD_RES 8: note = 6-aminohexanoic acid
  MOD_RES 20: note = 6-aminohexanoic acid
  MOD_RES 5: note = b-Ala
  MOD_RES 18: note = b-Ala
  MOD_RES 22: note = b-Ala
  MOD_RES 16: note = 6-aminohexanoic acid
RXRRBRRXRILFQYRXRBRXRBASSLNIAXC

SEQ ID NO: 331 - protein, 25 aa, Synthetic construct
  source 1..25: note = Cell-Penetrating Peptide
  MOD_RES 5: note = b-Ala
  MOD_RES 24: note = b-Ala
  MOD_RES 20: note = b-Ala
  MOD_RES 22: note = 6-aminohexanoic acid
  MOD_RES 18: note = 6-aminohexanoic acid
  MOD_RES 2: note = 6-aminohexanoic acid
  MOD_RES 8: note = 6-aminohexanoic acid
RXRRBRRXRASSLNIARXRBRXRBC

SEQ ID NO: 332 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 13: note = 6-aminohexanoic acid
  MOD_RES 5: note = b-Ala
  MOD_RES 11: note = b-Ala
  MOD_RES 14: note = b-Ala
RXRRBRRXRRBRXBASSLNIA

SEQ ID NO: 333 - protein, 12 aa, Synthetic construct
  source 1..12: note = Cell-Penetrating Peptide
THRPPMWSPVWP

SEQ ID NO: 334 - protein, 11 aa, Synthetic construct
  source 1..11: note = Cell-Penetrating Peptide
HRPPMWSPVWP

SEQ ID NO: 335 - protein, 10 aa, Synthetic construct
  source 1..10: note = Cell-Penetrating Peptide
THRPPMWSPV

SEQ ID NO: 336 - protein, 9 aa, Synthetic construct
  source 1..9: note = Cell-Penetrating Peptide
THRPPMWSP

SEQ ID NO: 337 - protein, 12 aa, Synthetic construct
  source 1..12: note = Cell-Penetrating Peptide
THRPPMWSPVFP

SEQ ID NO: 338 - protein, 12 aa, Synthetic construct
  source 1..12: note = Cell-Penetrating Peptide
THRPPMWSPVYP

SEQ ID NO: 339 - protein, 12 aa, Synthetic construct
  source 1..12: note = Cell-Penetrating Peptide
THRPPMWSPAWP

SEQ ID NO: 340 - protein, 12 aa, Synthetic construct
  source 1..12: note = Cell-Penetrating Peptide
THRPPMWSPLWP

SEQ ID NO: 341 - protein, 12 aa, Synthetic construct
  source 1..12: note = Cell-Penetrating Peptide
THRPPMWSPIWP

SEQ ID NO: 342 - protein, 13 aa, Synthetic construct
  source 1..13: note = Cell-Penetrating Peptide
THRPPMWTPVVWP

SEQ ID NO: 343 - protein, 12 aa, Synthetic construct
  source 1..12: note = Cell-Penetrating Peptide
THRPPMFSPVWP

SEQ ID NO: 344 - protein, 8 aa, Synthetic construct
  source 1..8: note = Cell-Penetrating Peptide
THRPPMWS

SEQ ID NO: 345 - protein, 10 aa, Synthetic construct
  source 1..10: note = Cell-Penetrating Peptide
HRPPMWSPVW

SEQ ID NO: 346 - protein, 12 aa, Synthetic construct
  source 1..12: note = Cell-Penetrating Peptide
THRPPMYSPVWP

SEQ ID NO: 347 - protein, 12 aa, Synthetic construct
  source 1..12: note = Cell-Penetrating Peptide
  SITE 6: note = Nle
THRPPXWSPVWP

SEQ ID NO: 348 - protein, 12 aa, Synthetic construct
  source 1..12: note = Cell-Penetrating Peptide
THKPPMWSPVWP

SEQ ID NO: 349 - protein, 12 aa, Synthetic construct
  source 1..12: note = Cell-Penetrating Peptide
SHRPPMWSPVWP

SEQ ID NO: 350 - protein, 7 aa, Synthetic construct
  source 1..7: note = Cell-Penetrating Peptide
STFTHPR

SEQ ID NO: 351 - protein, 7 aa, Synthetic construct
  source 1..7: note = Cell-Penetrating Peptide
YDIDNRR

SEQ ID NO: 352 - protein, 7 aa, Synthetic construct
  source 1..7: note = Cell-Penetrating Peptide
AYKPVGR

SEQ ID NO: 353 - protein, 7 aa, Synthetic construct
  source 1..7: note = Cell-Penetrating Peptide
HAIYPRH

SEQ ID NO: 354 - protein, 7 aa, Synthetic construct
  source 1..7: note = Cell-Penetrating Peptide
HTPNSTH

SEQ ID NO: 355 - protein, 7 aa, Synthetic construct
  source 1..7: note = Cell-Penetrating Peptide
ASSPVHR

SEQ ID NO: 356 - protein, 7 aa, Synthetic construct
  source 1..7: note = Cell-Penetrating Peptide
SSLPLRK

SEQ ID NO: 357 - protein, 4 aa, Synthetic construct
  source 1..4: note = Cell-Penetrating Peptide
KKRS

SEQ ID NO: 358 - protein, 4 aa, Synthetic construct
  source 1..4: note = Cell-Penetrating Peptide
KRSK

SEQ ID NO: 359 - protein, 5 aa, Synthetic construct
  source 1..5: note = Cell-Penetrating Peptide
KKRSK

SEQ ID NO: 360 - protein, 4 aa, Synthetic construct
  source 1..4: note = Cell-Penetrating Peptide
KSRK

SEQ ID NO: 361 - protein, 4 aa, Synthetic construct
  source 1..4: note = Cell-Penetrating Peptide
SRKR

SEQ ID NO: 362 - protein, 4 aa, Synthetic construct
  source 1..4: note = Cell-Penetrating Peptide
RKRK

SEQ ID NO: 363 - protein, 5 aa, Synthetic construct
  source 1..5: note = Cell-Penetrating Peptide
KSRKR

SEQ ID NO: 364 - protein, 7 aa, Synthetic construct
  source 1..7: note = Cell-Penetrating Peptide
QHPPWRV

SEQ ID NO: 365 - protein, 7 aa, Synthetic construct
  source 1..7: note = Cell-Penetrating Peptide
THPPTTH

SEQ ID NO: 366 - protein, 7 aa, Synthetic construct
  source 1..7: note = Cell-Penetrating Peptide
YKHTPTT

SEQ ID NO: 367 - protein, 7 aa, Synthetic construct
  source 1..7: note = Cell-Penetrating Peptide
QGMHRGT

SEQ ID NO: 368 - protein, 5 aa, Synthetic construct
  source 1..5: note = Cell-Penetrating Peptide
SRKRK

SEQ ID NO: 369 - protein, 6 aa, Synthetic construct
  source 1..6: note = Cell-Penetrating Peptide
KSRKRK

SEQ ID NO: 370 - protein, 7 aa, Synthetic construct
  source 1..7: note = Cell-Penetrating Peptide
PKKKRKV

SEQ ID NO: 371 - protein, 7 aa, Synthetic construct
  source 1..7: note = Cell-Penetrating Peptide
GKKRSKV

SEQ ID NO: 372 - protein, 7 aa, Synthetic construct
  source 1..7: note = Cell-Penetrating Peptide
KSRKRKL

SEQ ID NO: 373 - protein, 7 aa, Synthetic construct
  source 1..7: note = Cell-Penetrating Peptide
HSPSKIP

SEQ ID NO: 374 - protein, 7 aa, Synthetic construct
  source 1..7: note = Cell-Penetrating Peptide
HMATFHY

SEQ ID NO: 375 - protein, 7 aa, Synthetic construct
  source 1..7: note = Cell-Penetrating Peptide
AQPNKFK

SEQ ID NO: 376 - protein, 7 aa, Synthetic construct
  source 1..7: note = Cell-Penetrating Peptide
NLTRLHT

SEQ ID NO: 377 - protein, 4 aa, Synthetic construct
  source 1..4: note = Cell-Penetrating Peptide
KKKR

SEQ ID NO: 378 - protein, 4 aa, Synthetic construct
  source 1..4: note = Cell-Penetrating Peptide
KKRK

SEQ ID NO: 379 - protein, 5 aa, Synthetic construct
  source 1..5: note = Cell-Penetrating Peptide
KKKRK

SEQ ID NO: 380 - protein, 24 aa, Synthetic construct
  source 1..24: note = Cell-Penetrating Peptide
RRRRRRQIKIWFQNRRMKWKKGGC

SEQ ID NO: 381 - protein, 25 aa, Synthetic construct
  source 1..25: note = Cell-Penetrating Peptide
RRRRRRRQIKIWFQNRRMKWKKGGC

SEQ ID NO: 382 - protein, 19 aa, Synthetic construct
  source 1..19: note = Cell-Penetrating Peptide
RQIKIWFQNRRMKWKKGGC

SEQ ID NO: 383 - protein, 23 aa, Synthetic construct
  source 1..23: note = Cell-Penetrating Peptide
RRRRRRRQIKIWFQNRRMKWKKC

SEQ ID NO: 384 - protein, 28 aa, Synthetic construct
  source 1..28: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRRQIKIWFQNRRMKWKKGGC

SEQ ID NO: 385 - protein, 24 aa, Synthetic construct
  source 1..24: note = Cell-Penetrating Peptide
  MOD_RES 17: note = 6-aminohexanoic acid
  MOD_RES 19: note = 6-aminohexanoic acid
  MOD_RES 21: note = 6-aminohexanoic acid
  MOD_RES 23: note = 6-aminohexanoic acid
RRRRRRRQIKILFQNRXRXRXRXC

SEQ ID NO: 386 - protein, 13 aa, Synthetic construct
  source 1..13: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
RXRRXRRXRRXRC

SEQ ID NO: 387 - protein, 14 aa, Synthetic construct
  source 1..14: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 13: note = 6-aminohexanoic acid
RXRRXRRXRRXRXC

SEQ ID NO: 388 - protein, 26 aa, Synthetic construct
  source 1..26: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIKILFQNRRMKWKKGGC

SEQ ID NO: 389 - protein, 24 aa, Synthetic construct
  source 1..24: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIKILFQNRRMKWKKC

SEQ ID NO: 390 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIKILFQNRMKWKKC

SEQ ID NO: 391 - protein, 24 aa, Synthetic construct
  source 1..24: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIKILFQNXRMKWKKC

SEQ ID NO: 392 - protein, 24 aa, Synthetic construct
  source 1..24: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIKILFQNHRMKWKKC

SEQ ID NO: 393 - protein, 24 aa, Synthetic construct
  source 1..24: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIKILFQNXRMKWKAC

SEQ ID NO: 394 - protein, 25 aa, Synthetic construct
  source 1..25: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIKILFQNXRMKWHKAC

SEQ ID NO: 395 - protein, 24 aa, Synthetic construct
  source 1..24: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIKILFQNXRMKWHRC

SEQ ID NO: 396 - protein, 24 aa, Synthetic construct
  source 1..24: note = Cell-Penetrating Peptide
  MOD_RES 2: note = 6-aminohexanoic acid
  MOD_RES 4: note = 6-aminohexanoic acid
  MOD_RES 6: note = 6-aminohexanoic acid
  MOD_RES 8: note = 6-aminohexanoic acid
RXRXRXRXRIKILFQNRRMKWKKC

SEQ ID NO: 397 - protein, 24 aa, Synthetic construct
  source 1..24: note = Cell-Penetrating Peptide
RARARARARIKILFQNRRMKWKKC

SEQ ID NO: 398 - protein, 25 aa, Synthetic construct
  source 1..25: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 17: note = 6-aminohexanoic acid
RXRRXRRXRIXILFQNXRMKWHKAC

SEQ ID NO: 399 - protein, 25 aa, Synthetic construct
  source 1..25: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIHILFQNXRMKWHKAC

SEQ ID NO: 400 - protein, 25 aa, Synthetic construct
  source 1..25: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIRILFQNXRMKWHKAC

SEQ ID NO: 401 - protein, 25 aa, Synthetic construct
  source 1..25: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
  MOD_RES 11: note = 6-aminohexanoic acid
RXRRXRRXRIXILFQYXRMKWHKAC

SEQ ID NO: 402 - protein, 26 aa, Synthetic construct
  source 1..26: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 18: note = 6-aminohexanoic acid
RXRRXRRXRLYSPLSFQXRMKWHKAC

SEQ ID NO: 403 - protein, 25 aa, Synthetic construct
  source 1..25: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRISILFQYXRMKWHKAC

SEQ ID NO: 404 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 15: note = 6-aminohexanoic acid
RXRRXRRXRILFQYXRMKWHKAC

SEQ ID NO: 405 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 14: note = 6-aminohexanoic acid
RXRRXRIXILFQYXRMKWHKAC

SEQ ID NO: 406 - protein, 25 aa, Synthetic construct
  source 1..25: note = Cell-Penetrating Peptide
  MOD_RES 2: note = 6-aminohexanoic acid
  MOD_RES 8: note = 6-aminohexanoic acid
  MOD_RES 17: note = 6-aminohexanoic acid
RXRRARRXRIHILFQYXRMKWHKAC

SEQ ID NO: 407 - protein, 25 aa, Synthetic construct
  source 1..25: note = Cell-Penetrating Peptide
  MOD_RES 17: note = 6-aminohexanoic acid
  MOD_RES 5: note = 6-aminohexanoic acid
RARRXRRARIHILFQYXRMKWHKAC

SEQ ID NO: 408 - protein, 25 aa, Synthetic construct
  source 1..25: note = Cell-Penetrating Peptide
  MOD_RES 5: note = 6-aminohexanoic acid
  MOD_RES 8: note = 6-aminohexanoic acid
  MOD_RES 2: note = 6-aminohexanoic acid
  MOD_RES 17: note = 6-aminohexanoic acid
RXRRXRRXRIHILFQYXRMKWHKAC

SEQ ID NO: 409 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIKILFQNRRMKWHK

SEQ ID NO: 410 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIKILFQNXRMKWHK

SEQ ID NO: 411 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
RXRRXRRXRIXILFQNRRMKWHK

SEQ ID NO: 412 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 17: note = 6-aminohexanoic acid
RXRRXRRXRIXILFQNXRMKWHK

SEQ ID NO: 413 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIHILFQNRRMKWHK

SEQ ID NO: 414 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIHILFQNXRMKWHK

SEQ ID NO: 415 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIRILFQNRRMKWHK

SEQ ID NO: 416 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIRILFQNXRMKWHK

SEQ ID NO: 417 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIILFQNRRMKWHK

SEQ ID NO: 418 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
RXRRXRRXRIILFQNXRMKWHK

SEQ ID NO: 419 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRKILFQNRRMKWHK

SEQ ID NO: 420 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
RXRRXRRXRKILFQNXRMKWHK

SEQ ID NO: 421 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 10: note = 6-aminohexanoic acid
RXRRXRRXRXILFQNRRMKWHK

SEQ ID NO: 422 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 10: note = 6-aminohexanoic acid
  MOD_RES 16: note = 6-aminohexanoic acid
RXRRXRRXRXILFQNXRMKWHK

SEQ ID NO: 423 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRHILFQNRRMKWHK

SEQ ID NO: 424 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
RXRRXRRXRHILFQNXRMKWHK

SEQ ID NO: 425 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRRILFQNRRMKWHK

SEQ ID NO: 426 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
RXRRXRRXRRILFQNXRMKWHK

SEQ ID NO: 427 - protein, 21 aa, Synthetic construct
  source 1..21: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRILFQNRRMKWHK

SEQ ID NO: 428 - protein, 21 aa, Synthetic construct
  source 1..21: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 15: note = 6-aminohexanoic acid
RXRRXRRXRILFQNXRMKWHK

SEQ ID NO: 429 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIKILFQYRRMKWHK

SEQ ID NO: 430 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIKILFQYXRMKWHK

SEQ ID NO: 431 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
RXRRXRRXRIXILFQYRRMKWHK

SEQ ID NO: 432 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 17: note = 6-aminohexanoic acid
RXRRXRRXRIXILFQYXRMKWHK

SEQ ID NO: 433 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIHILFQYRRMKWHK

SEQ ID NO: 434 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIHILFQYXRMKWHK

SEQ ID NO: 435 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIRILFQYRRMKWHK

SEQ ID NO: 436 - protein, 23 aa, Synthetic construct
  source 1..23: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 17: note = 6-aminohexanoic acid
RXRRXRRXRIRILFQYXRMKWHK

SEQ ID NO: 437 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRIILFQYRRMKWHK

SEQ ID NO: 438 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
RXRRXRRXRIILFQYXRMKWHK

SEQ ID NO: 439 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRKILFQYRRMKWHK

SEQ ID NO: 440 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
RXRRXRRXRKILFQYXRMKWHK

SEQ ID NO: 441 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 10: note = 6-aminohexanoic acid
RXRRXRRXRXILFQYRRMKWHK

SEQ ID NO: 442 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
  MOD_RES 10: note = 6-aminohexanoic acid
RXRRXRRXRXILFQYXRMKWHK

SEQ ID NO: 443 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRHILFQYRRMKWHK

SEQ ID NO: 444 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
RXRRXRRXRHILFQYXRMKWHK

SEQ ID NO: 445 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRRILFQYRRMKWHK

SEQ ID NO: 446 - protein, 22 aa, Synthetic construct
  source 1..22: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 16: note = 6-aminohexanoic acid
RXRRXRRXRRILFQYXRMKWHK

SEQ ID NO: 447 - protein, 21 aa, Synthetic construct
  source 1..21: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
RXRRXRRXRILFQYRRMKWHK

SEQ ID NO: 448 - protein, 21 aa, Synthetic construct
  source 1..21: note = Cell-Penetrating Peptide
  MOD_RES 2: note = 6-aminohexanoic acid
  MOD_RES 5: note = 6-aminohexanoic acid
  MOD_RES 8: note = 6-aminohexanoic acid
  MOD_RES 15: note = 6-aminohexanoic acid
RXRRXRRXRILFQYXRMKWHK

SEQ ID NO: 449 - protein, 6 aa, Synthetic construct
  source 1..6: note = Cell-Penetrating Peptide
RARRAR

SEQ ID NO: 450 - protein, 9 aa, Synthetic construct
  source 1..9: note = Cell-Penetrating Peptide
RARRARRAR

SEQ ID NO: 451 - protein, 12 aa, Synthetic construct
  source 1..12: note = Cell-Penetrating Peptide
RARRARRARRAR

SEQ ID NO: 452 - protein, 7 aa, Synthetic construct
  source 1..7: note = Cell-Penetrating Peptide
  MOD_RES 2: note = 6-aminohexanoic acid
  MOD_RES 5: note = 6-aminohexanoic acid
RXRRXRI

SEQ ID NO: 453 - protein, 9 aa, Synthetic construct
  source 1..9: note = Cell-Penetrating Peptide
  MOD_RES 2: note = 6-aminohexanoic acid
  MOD_RES 8: note = 6-aminohexanoic acid
RXRRARRXR

SEQ ID NO: 454 - protein, 9 aa, Synthetic construct
  source 1..9: note = Cell-Penetrating Peptide
  MOD_RES 5: note = 6-aminohexanoic acid
RARRXRRAR

SEQ ID NO: 455 - protein, 7 aa, Synthetic construct
  source 1..7: note = Cell-Penetrating Peptide
RRMKWHK

SEQ ID NO: 456 - protein, 7 aa, Synthetic construct
  source 1..7: note = Cell-Penetrating Peptide
  MOD_RES 1: note = 6-aminohexanoic acid
XRMKWHK

SEQ ID NO: 457 - protein, 26 aa, Synthetic construct
  source 1..26: note = Cell-Penetrating Peptide
  MOD_RES 1..14: note = 6-aminohexanoic acid
  MOD_RES 19..20: note = 6-aminohexanoic acid
XXXXXXXXXXXXXXILFQXXRMKWHK

SEQ ID NO: 458 - protein, 25 aa, Synthetic construct
  source 1..25: note = Cell-Penetrating Peptide
RRRRRRRQIKILFQNPKKKRKVGGC

SEQ ID NO: 459 - protein, 16 aa, Synthetic construct
  source 1..16: note = Cell-Penetrating Peptide
HHFFRRRRRRRRRFFC

SEQ ID NO: 460 - protein, 18 aa, Synthetic construct
  source 1..18: note = Cell-Penetrating Peptide
HHHHHHRRRRRRRRRFFC

SEQ ID NO: 461 - protein, 20 aa, Synthetic construct
  source 1..20: note = Cell-Penetrating Peptide
HHHHHHFFRRRRRRRRRFFC

SEQ ID NO: 462 - protein, 19 aa, Synthetic construct
  source 1..19: note = Cell-Penetrating Peptide
  MOD_RES 6..7: note = 6-aminohexanoic acid
HHHHHXXRRRRRRRRRFFC

SEQ ID NO: 463 - protein, 22 aa, Synthetic construct
  source 1..22: note = Cell-Penetrating Peptide
  MOD_RES 7..8: note = 6-aminohexanoic acid
HHHHHHXXFFRRRRRRRRRFFC

SEQ ID NO: 464 - protein, 20 aa, Synthetic construct
  source 1..20: note = Cell-Penetrating Peptide
  MOD_RES 16: note = 6-aminohexanoic acid
  MOD_RES 4: note = 6-aminohexanoic acid
HHHXRRRRRRRRRFFXHHHC

SEQ ID NO: 465 - protein, 7 aa, Synthetic construct
  source 1..7: note = Cell-Penetrating Peptide
  MOD_RES 1: note = 6-aminohexanoic acid
XRWKWHK

SEQ ID NO: 466 - protein, 7 aa, Synthetic construct
  source 1..7: note = Cell-Penetrating Peptide
  MOD_RES 2: note = 6-aminohexanoic acid
  MOD_RES 6: note = 6-aminohexanoic acid
RXRARXR

SEQ ID NO: 467 - protein, 7 aa, Synthetic construct
  source 1..7: note = Cell-Penetrating Peptide; note = Cell-Penetrating Peptide
  MOD_RES 2: note = 6-aminohexanoic acid
  MOD_RES 4: note = 6-aminohexanoic acid
  MOD_RES 6: note = 6-aminohexanoic acid
RXRXRXR

SEQ ID NO: 468 - protein, 7 aa, Synthetic construct
  source 1..7: note = Cell-Penetrating Peptide
  MOD_RES 4: note = 6-aminohexanoic acid
RARXRAR

SEQ ID NO: 469 - protein, 5 aa, Synthetic construct
  source 1..5: note = Cell-Penetrating Peptide
  MOD_RES 2: note = 6-aminohexanoic acid
RXRAR

SEQ ID NO: 470 - protein, 26 aa, Synthetic construct
  source 1..26: note = Cell-Penetrating Peptide
  MOD_RES 1..14: note = 6-aminohexanoic acid
  MOD_RES 19..20: note = 6-aminohexanoic acid
XXXXXXXXXXXXXXILFQXXHMKWHK

SEQ ID NO: 471 - protein, 26 aa, Synthetic construct
  source 1..26: note = Cell-Penetrating Peptide
  MOD_RES 1..14: note = 6-aminohexanoic acid
  MOD_RES 19..20: note = 6-aminohexanoic acid
XXXXXXXXXXXXXXILFQXXRWKWHK

SEQ ID NO: 472 - protein, 26 aa, Synthetic construct
  source 1..26: note = Cell-Penetrating Peptide
  MOD_RES 1..14: note = 6-aminohexanoic acid
  MOD_RES 19..20: note = 6-aminohexanoic acid
XXXXXXXXXXXXXXILFQXXHWKWHK

SEQ ID NO: 473 - protein, 26 aa, Synthetic construct
  source 1..26: note = Cell-Penetrating Peptide
  MOD_RES 1..14: note = 6-aminohexanoic acid
  MOD_RES 19: note = 6-aminohexanoic acid
  MOD_RES 21: note = 6-aminohexanoic acid
  MOD_RES 25: note = 6-aminohexanoic acid
XXXXXXXXXXXXXXILFQXRXRARXR

SEQ ID NO: 474 - protein, 26 aa, Synthetic construct
  source 1..26: note = Cell-Penetrating Peptide
  MOD_RES 1..14: note = 6-aminohexanoic acid
  MOD_RES 19: note = 6-aminohexanoic acid
  MOD_RES 21: note = 6-aminohexanoic acid
  MOD_RES 23: note = 6-aminohexanoic acid
  MOD_RES 25: note = 6-aminohexanoic acid
XXXXXXXXXXXXXXILFQXRXRXRXR

SEQ ID NO: 475 - protein, 25 aa, Synthetic construct
  source 1..25: note = Cell-Penetrating Peptide
  MOD_RES 1..14: note = 6-aminohexanoic acid
  MOD_RES 19: note = 6-aminohexanoic acid
  MOD_RES 21: note = 6-aminohexanoic acid
  MOD_RES 24: note = 6-aminohexanoic acid
XXXXXXXXXXXXXXILFQXRXRRXR

SEQ ID NO: 476 - protein, 26 aa, Synthetic construct
  source 1..26: note = Cell-Penetrating Peptide
  MOD_RES 1..14: note = 6-aminohexanoic acid
  MOD_RES 19: note = 6-aminohexanoic acid
  MOD_RES 23: note = 6-aminohexanoic acid
XXXXXXXXXXXXXXILFQXRARXRAR

SEQ ID NO: 477 - protein, 26 aa, Synthetic construct
  source 1..26: note = Cell-Penetrating Peptide
  MOD_RES 1..14: note = 6-aminohexanoic acid
  MOD_RES 19..20: note = 6-aminohexanoic acid
XXXXXXXXXXXXXXILIQXXRMKWHK

SEQ ID NO: 478 - protein, 26 aa, Synthetic construct
  source 1..26: note = Cell-Penetrating Peptide
  MOD_RES 1..14: note = 6-aminohexanoic acid
  MOD_RES 19..20: note = 6-aminohexanoic acid
XXXXXXXXXXXXXXILIQXXHMKWHK

SEQ ID NO: 479 - protein, 26 aa, Synthetic construct
  source 1..26: note = Cell-Penetrating Peptide
  MOD_RES 1..14: note = 6-aminohexanoic acid
  MOD_RES 19..20: note = 6-aminohexanoic acid
XXXXXXXXXXXXXXILIQXXRWKWHK

SEQ ID NO: 480 - protein, 26 aa, Synthetic construct
  source 1..26: note = Cell-Penetrating Peptide
  MOD_RES 1..14: note = 6-aminohexanoic acid
  MOD_RES 19: note = 6-aminohexanoic acid
  MOD_RES 21: note = 6-aminohexanoic acid
  MOD_RES 25: note = 6-aminohexanoic acid
XXXXXXXXXXXXXXILIQXRXRARXR

SEQ ID NO: 481 - protein, 25 aa, Synthetic construct
  source 1..25: note = Cell-Penetrating Peptide
  MOD_RES 1..14: note = 6-aminohexanoic acid
  MOD_RES 19: note = 6-aminohexanoic acid
  MOD_RES 21: note = 6-aminohexanoic acid
  MOD_RES 24: note = 6-aminohexanoic acid
XXXXXXXXXXXXXXILIQXRXRRXR

SEQ ID NO: 482 - protein, 26 aa, Synthetic construct
  source 1..26: note = Cell-Penetrating Peptide
  MOD_RES 1..14: note = 6-aminohexanoic acid
  MOD_RES 19: note = 6-aminohexanoic acid
  MOD_RES 23: note = 6-aminohexanoic acid
XXXXXXXXXXXXXXILIQXRARXRAR

SEQ ID NO: 483 - protein, 24 aa, Synthetic construct
  source 1..24: note = Cell-Penetrating Peptide
  MOD_RES 1..14: note = 6-aminohexanoic acid
  MOD_RES 19: note = 6-aminohexanoic acid
  MOD_RES 21: note = 6-aminohexanoic acid
XXXXXXXXXXXXXXILIQXRXRAR

SEQ ID NO: 484 - protein, 24 aa, Synthetic construct
  source 1..24: note = Cell-Penetrating Peptide
  MOD_RES 1..14: note = 6-aminohexanoic acid
  MOD_RES 19: note = 6-aminohexanoic acid
  MOD_RES 21: note = 6-aminohexanoic acid
XXXXXXXXXXXXXXILFQXRXRAR

SEQ ID NO: 485 - protein, 26 aa, Synthetic construct
  source 1..26: note = Cell-Penetrating Peptide
  MOD_RES 1..14: note = 6-aminohexanoic acid
  MOD_RES 19..20: note = 6-aminohexanoic acid
XXXXXXXXXXXXXXILIQXXHWKWHK

SEQ ID NO: 486 - protein, 26 aa, Synthetic construct
  source 1..26: note = Cell-Penetrating Peptide
  MOD_RES 1..14: note = 6-aminohexanoic acid
  MOD_RES 19: note = 6-aminohexanoic acid
  MOD_RES 21: note = 6-aminohexanoic acid
  MOD_RES 23: note = 6-aminohexanoic acid
  MOD_RES 25: note = 6-aminohexanoic acid
XXXXXXXXXXXXXXILIQXRXRXRXR

SEQ ID NO: 487 - protein, 14 aa, Synthetic construct
  source 1..14: note = Cell-Penetrating Peptide
  MOD_RES 2: note = 6-aminohexanoic acid
  MOD_RES 8: note = 6-aminohexanoic acid
  MOD_RES 13: note = 6-aminohexanoic acid
RXRRARRXRRARXA

SEQ ID NO: 488 - protein, 23 aa, Synthetic construct
  source 1..23: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILFQYXHMKWHKAC

SEQ ID NO: 489 - protein, 23 aa, Synthetic construct
  source 1..23: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILFQYXRMKWHKAC

SEQ ID NO: 490 - protein, 23 aa, Synthetic construct
  source 1..23: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILFQYXRWKWHKAC

SEQ ID NO: 491 - protein, 23 aa, Synthetic construct
  source 1..23: note = Cell-Penetrating Peptide
  MOD_RES 5: note = 6-aminohexanoic acid
  MOD_RES 15: note = 6-aminohexanoic acid
RARRXRRARILFQYXRMKWHKAC

SEQ ID NO: 492 - protein, 23 aa, Synthetic construct
  source 1..23: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILFQYXHWKWHKAC

SEQ ID NO: 493 - protein, 23 aa, Synthetic construct
  source 1..23: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 20: note = 6-aminohexanoic acid
RXRRARRXRILFQYRXRARXRAC

SEQ ID NO: 494 - protein, 23 aa, Synthetic construct
  source 1..23: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 18: note = 6-aminohexanoic acid
  MOD_RES 20: note = 6-aminohexanoic acid
RXRRARRXRILFQYRXRXRXRAC

SEQ ID NO: 495 - protein, 23 aa, Synthetic construct
  source 1..23: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILIQYXRMKWHKAC

SEQ ID NO: 496 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
  MOD_RES 16: note = 6-aminohexanoic acid
RXRRXRILFQYRXRRXRC

SEQ ID NO: 497 - protein, 20 aa, Synthetic construct
  source 1..20: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
  MOD_RES 16: note = 6-aminohexanoic acid
RXRRXRILFQYRXRRXRCYS

SEQ ID NO: 498 - protein, 23 aa, Synthetic construct
  source 1..23: note = Cell-Penetrating Peptide
  MOD_RES 5: note = 6-aminohexanoic acid
  MOD_RES 18: note = 6-aminohexanoic acid
RARRXRRARILFQYRARXRARAC

SEQ ID NO: 499 - protein, 23 aa, Synthetic construct
  source 1..23: note = Cell-Penetrating Peptide
  MOD_RES 5: note = 6-aminohexanoic acid
  MOD_RES 16: note = 6-aminohexanoic acid
  MOD_RES 20: note = 6-aminohexanoic acid
RARRXRRARILFQYRXRARXRAC

SEQ ID NO: 500 - protein, 22 aa, Synthetic construct
  source 1..22: note = Cell-Penetrating Peptide
  MOD_RES 5: note = 6-aminohexanoic acid
  MOD_RES 16: note = 6-aminohexanoic acid
  MOD_RES 19: note = 6-aminohexanoic acid
RARRXRRARILFQYRXRRXRAC

SEQ ID NO: 501 - protein, 22 aa, Synthetic construct
  source 1..22: note = Cell-Penetrating Peptide
  MOD_RES 5: note = 6-aminohexanoic acid
  MOD_RES 16: note = 6-aminohexanoic acid
  MOD_RES 20: note = 6-aminohexanoic acid
RARRXRRARILFQYRXRARXAC

SEQ ID NO: 502 - protein, 22 aa, Synthetic construct
  source 1..22: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 19: note = 6-aminohexanoic acid
RXRRARRXRILFQYRXRRXRAC

SEQ ID NO: 503 - protein, 22 aa, Synthetic construct
  source 1..22: note = Cell-Penetrating Peptide
  MOD_RES 2: note = 6-aminohexanoic acid
  MOD_RES 8: note = 6-aminohexanoic acid
  MOD_RES 20: note = 6-aminohexanoic acid
  MOD_RES 16: note = 6-aminohexanoic acid
RXRRARRXRILFQYRXRARXAC

SEQ ID NO: 504 - protein, 25 aa, Synthetic construct
  source 1..25: note = Cell-Penetrating Peptide
  MOD_RES 2: note = 6-aminohexanoic acid
  MOD_RES 8: note = 6-aminohexanoic acid
  MOD_RES 17: note = 6-aminohexanoic acid
RXRRARRXRIHILFQNXRMKWHKAC

SEQ ID NO: 505 - protein, 15 aa, Synthetic construct
  source 1..15: note = Cell-Penetrating Peptide
  MOD_RES 2: note = 6-aminohexanoic acid
  MOD_RES 8: note = 6-aminohexanoic acid
  MOD_RES 13: note = 6-aminohexanoic acid
RXRRARRXRRARXAC

SEQ ID NO: 506 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILFQYXHMKWHK

SEQ ID NO: 507 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILFQYXRMKWHK

SEQ ID NO: 508 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILFQYXRWKWHK

SEQ ID NO: 509 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 20: note = 6-aminohexanoic acid
RXRRARRXRILFQYRXRARXR

SEQ ID NO: 510 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 18: note = 6-aminohexanoic acid
  MOD_RES 20: note = 6-aminohexanoic acid
RXRRARRXRILFQYRXRXRXR

SEQ ID NO: 511 - protein, 20 aa, Synthetic construct
  source 1..20: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 19: note = 6-aminohexanoic acid
RXRRARRXRILFQYRXRRXR

SEQ ID NO: 512 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 18: note = 6-aminohexanoic acid
RXRRARRXRILFQYRARXRAR

SEQ ID NO: 513 - protein, 19 aa, Synthetic construct
  source 1..19: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
RXRRARRXRILFQYRXRAR

SEQ ID NO: 514 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILIQYXHMKWHK

SEQ ID NO: 515 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILIQYXRMKWHK

SEQ ID NO: 516 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 15: note = 6-aminohexanoic acid
RXRRARRXRILIQYXRWKWHK

SEQ ID NO: 517 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 20: note = 6-aminohexanoic acid
RXRRARRXRILIQYRXRARXR

SEQ ID NO: 518 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 18: note = 6-aminohexanoic acid
  MOD_RES 20: note = 6-aminohexanoic acid
RXRRARRXRILIQYRXRXRXR

SEQ ID NO: 519 - protein, 20 aa, Synthetic construct
  source 1..20: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
  MOD_RES 19: note = 6-aminohexanoic acid
RXRRARRXRILIQYRXRRXR

SEQ ID NO: 520 - protein, 21 aa, Synthetic construct
  source 1..21: note = Cell-Penetrating Peptide
  MOD_RES 2: note = 6-aminohexanoic acid
  MOD_RES 8: note = 6-aminohexanoic acid
  MOD_RES 18: note = 6-aminohexanoic acid
RXRRARRXRILIQYRARXRAR

SEQ ID NO: 521 - protein, 19 aa, Synthetic construct
  source 1..19: note = Cell-Penetrating Peptide
  MOD_RES 2: note = 6-aminohexanoic acid
  MOD_RES 8: note = 6-aminohexanoic acid
  MOD_RES 16: note = 6-aminohexanoic acid
RXRRARRXRILIQYRXRAR

SEQ ID NO: 522 - protein, 21 aa, Synthetic construct
  source 1..21: note = Cell-Penetrating Peptide
  MOD_RES 5: note = 6-aminohexanoic acid
  MOD_RES 15: note = 6-aminohexanoic acid
RARRXRRARILFQYXHMKWHK

SEQ ID NO: 523 - protein, 21 aa, Synthetic construct
  source 1..21: note = Cell-Penetrating Peptide
  MOD_RES 5: note = 6-aminohexanoic acid
  MOD_RES 15: note = 6-aminohexanoic acid
RARRXRRARILFQYXRMKWHK

SEQ ID NO: 524 - protein, 21 aa, Synthetic construct
  source 1..21: note = Cell-Penetrating Peptide
  MOD_RES 5: note = 6-aminohexanoic acid
  MOD_RES 15: note = 6-aminohexanoic acid
RARRXRRARILFQYXRWKWHK

SEQ ID NO: 525 - protein, 21 aa, Synthetic construct
  source 1..21: note = Cell-Penetrating Peptide
  MOD_RES 5: note = 6-aminohexanoic acid
  MOD_RES 16: note = 6-aminohexanoic acid
  MOD_RES 20: note = 6-aminohexanoic acid
RARRXRRARILFQYRXRARXR

SEQ ID NO: 526 - protein, 21 aa, Synthetic construct
  source 1..21: note = Cell-Penetrating Peptide
  MOD_RES 5: note = 6-aminohexanoic acid
  MOD_RES 16: note = 6-aminohexanoic acid
  MOD_RES 18: note = 6-aminohexanoic acid
  MOD_RES 20: note = 6-aminohexanoic acid
RARRXRRARILFQYRXRXRXR

SEQ ID NO: 527 - protein, 20 aa, Synthetic construct
  source 1..20: note = Cell-Penetrating Peptide
  MOD_RES 5: note = 6-aminohexanoic acid
  MOD_RES 16: note = 6-aminohexanoic acid
  MOD_RES 19: note = 6-aminohexanoic acid
RARRXRRARILFQYRXRRXR

SEQ ID NO: 528 - protein, 21 aa, Synthetic construct
  source 1..21: note = Cell-Penetrating Peptide
  MOD_RES 5: note = 6-aminohexanoic acid
  MOD_RES 18: note = 6-aminohexanoic acid
RARRXRRARILFQYRARXRAR

SEQ ID NO: 529 - protein, 19 aa, Synthetic construct
  source 1..19: note = Cell-Penetrating Peptide
  MOD_RES 5: note = 6-aminohexanoic acid
  MOD_RES 16: note = 6-aminohexanoic acid
RARRXRRARILFQYRXRAR

SEQ ID NO: 530 - protein, 21 aa, Synthetic construct
  source 1..21: note = Cell-Penetrating Peptide
  MOD_RES 5: note = 6-aminohexanoic acid
  MOD_RES 15: note = 6-aminohexanoic acid
RARRXRRARILIQYXHMKWHK

SEQ ID NO: 531 - protein, 21 aa, Synthetic construct
  source 1..21: note = Cell-Penetrating Peptide
  MOD_RES 5: note = 6-aminohexanoic acid
  MOD_RES 15: note = 6-aminohexanoic acid
RARRXRRARILIQYXRMKWHK

SEQ ID NO: 532 - protein, 21 aa, Synthetic construct
  source 1..21: note = Cell-Penetrating Peptide
  MOD_RES 5: note = 6-aminohexanoic acid
  MOD_RES 15: note = 6-aminohexanoic acid
RARRXRRARILIQYXRWKWHK

SEQ ID NO: 533 - protein, 21 aa, Synthetic construct
  source 1..21: note = Cell-Penetrating Peptide
  MOD_RES 5: note = 6-aminohexanoic acid
  MOD_RES 16: note = 6-aminohexanoic acid
  MOD_RES 20: note = 6-aminohexanoic acid
RARRXRRARILIQYRXRARXR

SEQ ID NO: 534 - protein, 21 aa, Synthetic construct
  source 1..21: note = Cell-Penetrating Peptide
  MOD_RES 5: note = 6-aminohexanoic acid
  MOD_RES 16: note = 6-aminohexanoic acid
  MOD_RES 18: note = 6-aminohexanoic acid
  MOD_RES 20: note = 6-aminohexanoic acid
RARRXRRARILIQYRXRXRXR

SEQ ID NO: 535 - protein, 20 aa, Synthetic construct
  source 1..20: note = Cell-Penetrating Peptide
  MOD_RES 5: note = 6-aminohexanoic acid
  MOD_RES 16: note = 6-aminohexanoic acid
  MOD_RES 19: note = 6-aminohexanoic acid
RARRXRRARILIQYRXRRXR

SEQ ID NO: 536 - protein, 21 aa, Synthetic construct
  source 1..21: note = Cell-Penetrating Peptide
  MOD_RES 5: note = 6-aminohexanoic acid
  MOD_RES 18: note = 6-aminohexanoic acid
RARRXRRARILIQYRARXRAR

SEQ ID NO: 537 - protein, 19 aa, Synthetic construct
  source 1..19: note = Cell-Penetrating Peptide
  MOD_RES 5: note = 6-aminohexanoic acid
  MOD_RES 16: note = 6-aminohexanoic acid
RARRXRRARILIQYRXRAR

SEQ ID NO: 538 - protein, 18 aa, Synthetic construct
  source 1..18: note = Cell-Penetrating Peptide
  MOD_RES 5: note = 6-aminohexanoic acid
  MOD_RES 12: note = 6-aminohexanoic acid
  MOD_RES 2: note = 6-aminohexanoic acid
RXRRXRILFQYXHMKWHK

SEQ ID NO: 539 - protein, 18 aa, Synthetic construct
  source 1..18: note = Cell-Penetrating Peptide
  MOD_RES 5: note = 6-aminohexanoic acid
  MOD_RES 12: note = 6-aminohexanoic acid
  MOD_RES 2: note = 6-aminohexanoic acid
RXRRXRILFQYXRMKWHK

SEQ ID NO: 540 - protein, 18 aa, Synthetic construct
  source 1..18: note = Cell-Penetrating Peptide
  MOD_RES 5: note = 6-aminohexanoic acid
  MOD_RES 2: note = 6-aminohexanoic acid
  MOD_RES 12: note = 6-aminohexanoic acid
RXRRXRILFQYXRWKWHK

SEQ ID NO: 541 - protein, 18 aa, Synthetic construct
  source 1..18: note = Cell-Penetrating Peptide
  MOD_RES 5: note = 6-aminohexanoic acid
  MOD_RES 2: note = 6-aminohexanoic acid
  MOD_RES 13: note = 6-aminohexanoic acid
  MOD_RES 17: note = 6-aminohexanoic acid
RXRRXRILFQYRXRARXR

SEQ ID NO: 542 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
  MOD_RES 15: note = 6-aminohexanoic acid
  MOD_RES 17: note = 6-aminohexanoic acid
RXRRXRILFQYRXRXRXR

SEQ ID NO: 543 - protein, 17 aa, Synthetic construct
  source 1..17: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
  MOD_RES 16: note = 6-aminohexanoic acid
RXRRXRILFQYRXRRXR

SEQ ID NO: 544 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 15: note = 6-aminohexanoic acid
RXRRXRILFQYRARXRAR

SEQ ID NO: 545 - protein, 16 aa, Synthetic construct
  source 1..16: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
RXRRXRILFQYRXRAR

SEQ ID NO: 546 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 12: note = 6-aminohexanoic acid
RXRRXRILIQYXHMKWHK

SEQ ID NO: 547 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 12: note = 6-aminohexanoic acid
RXRRXRILIQYXRMKWHK

SEQ ID NO: 548 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 12: note = 6-aminohexanoic acid
RXRRXRILIQYXRWKWHK

SEQ ID NO: 549 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
  MOD_RES 17: note = 6-aminohexanoic acid
RXRRXRILIQYRXRARXR

SEQ ID NO: 550 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
  MOD_RES 15: note = 6-aminohexanoic acid
  MOD_RES 17: note = 6-aminohexanoic acid
RXRRXRILIQYRXRXRXR

SEQ ID NO: 551 - protein, 17 aa, Synthetic construct
  source 1..17: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
  MOD_RES 16: note = 6-aminohexanoic acid
RXRRXRILIQYRXRRXR

SEQ ID NO: 552 - protein, 18 aa, Synthetic construct
  source 1..18: note = Cell-Penetrating Peptide
  MOD_RES 2: note = 6-aminohexanoic acid
  MOD_RES 5: note = 6-aminohexanoic acid
  MOD_RES 15: note = 6-aminohexanoic acid
RXRRXRILIQYRARXRAR

SEQ ID NO: 553 - protein, 16 aa, Synthetic construct
  source 1..16: note = Cell-Penetrating Peptide
  MOD_RES 2: note = 6-aminohexanoic acid
  MOD_RES 5: note = 6-aminohexanoic acid
  MOD_RES 13: note = 6-aminohexanoic acid
RXRRXRILIQYRXRAR

SEQ ID NO: 554 - protein, 17 aa, Synthetic construct
  source 1..17: note = Cell-Penetrating Peptide
  MOD_RES 4..14: note = 6-aminohexanoic acid
PRPXXXXXXXXXXXPRG

SEQ ID NO: 555 - protein, 7 aa, Synthetic construct
  source 1..7: note = Cell-Penetrating Peptide
RRMKWKK

SEQ ID NO: 556 - protein, 32 aa, Synthetic construct
  source 1..32: note = Cell-Penetrating Peptide
CKDEPQRRSARLSAKPAPPKPEPKPKKAPAKK

SEQ ID NO: 557 - protein, 8 aa, Synthetic construct
  source 1..8: note = Cell-Penetrating Peptide
RKKRRQRR

SEQ ID NO: 558 - protein, 7 aa, Synthetic construct
  source 1..7: note = Cell-Penetrating Peptide
RKKRRQR

SEQ ID NO: 559 - protein, 8 aa, Synthetic construct
  source 1..8: note = Cell-Penetrating Peptide
KKRRQRRR

SEQ ID NO: 560 - protein, 9 aa, Synthetic construct
  source 1..9: note = Cell-Penetrating Peptide
AKKRRQRRR

SEQ ID NO: 561 - protein, 9 aa, Synthetic construct
  source 1..9: note = Cell-Penetrating Peptide
RAKRRQRRR

SEQ ID NO: 562 - protein, 9 aa, Synthetic construct
  source 1..9: note = Cell-Penetrating Peptide
RKARRQRRR

SEQ ID NO: 563 - protein, 9 aa, Synthetic construct
  source 1..9: note = Cell-Penetrating Peptide
RKKARQRRR

SEQ ID NO: 564 - DNA, 21 bp, Synthetic construct
  source 1..21: mol_type = other DNA; note = Target Region Base Sequence
gactgggtgtctgtagtgacc